(19) United States
(12) Patent Application Publication
(10) Pub. No.: US 2023/0226193 A1
(43) Pub. Date: Jul. 20, 2023

(54) LIPID CONJUGATES FOR THE DELIVERY OF THERAPEUTIC AGENTS

(71) Applicant: Arrowhead Pharmaceuticals, Inc., Pasadena, CA (US)

(72) Inventors: Xiaokai Li, Middleton, WI (US); Tao Pei, Middleton, WI (US); Teng Ai, Middleton, WI (US); Susan Phan, Fitchburg, WI (US); Susan Ramos-Hunter, Hendersonville, TN (US)

(21) Appl. No.: 18/178,242

(22) Filed: Mar. 3, 2023

Related U.S. Application Data
(63) Continuation of application No. PCT/US2021/049880, filed on Sep. 10, 2021.
(60) Provisional application No. 63/230,257, filed on Aug. 6, 2021.
(60) Provisional application No. 63/214,745, filed on Jun. 24, 2021.
(60) Provisional application No. 63/077,290, filed on Sep. 11, 2020.

(51) Int. Cl. A61K 47/54; A61K 47/60; C12N 15/113

(52) U.S. Cl. CPC A61K 47/543; A61K 47/60; A61K 47/545; C12N 15/1136; C12N 2310/14; C12N 2310/3515; C12N 2320/32

(57) ABSTRACT

Disclosed herein are compounds according to Formula (I) comprising PK/PD modulators for delivery of oligonucleotide-based agents, e.g., double stranded RNAi agents, to certain cell types, such for example skeletal muscle cells, in vivo. The PK/PD modulators disclosed herein, when conjugated to an oligonucleotide-based therapeutic or diagnostic agent, such as an RNAi agent, can enhance the delivery of the composition to the specified cells being targeted to facilitate the inhibition of gene expression in those cells.

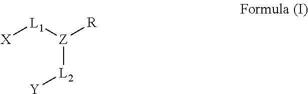

### CROSS REFERENCE TO RELATED APPLICATIONS

This application claims the benefit of priority of PCT application PCT/US2021/049880, filed on Sep. 10, 2021, which claims the benefit of priority of U.S. provisional application No. 63/077,290, filed on Sep. 11, 2020, U.S. provisional application No. 63/214,745, filed on Jun. 24, 2021, and U.S. provisional application No. 63/230,257, filed on Aug. 6, 2021. Each of these documents is hereby incorporated by reference in its entirety.

### FIELD OF THE INVENTION

The present disclosure relates to lipid conjugates (also referred to herein as lipid PK/PD modulators) for the delivery of oligonucleotide-based agents, e.g., double stranded RNAi agents, to certain cell types (e.g., skeletal muscle cells) in vivo, for inhibition of genes that are expressed in those cells.

### BACKGROUND

Oligonucleotide-based agents, such as antisense agents and double stranded RNA interference (RNAi) agents, have shown great promise and have the potential to revolutionize the field of medicine and the availability to patients of potent therapeutic treatment options. However, the effective delivery of oligonucleotide-based agents, and double-stranded therapeutic RNAi agents in particular, has long been a challenge in developing viable therapeutic pharmaceutical agents. This is particularly the case when trying to achieve specific and selective delivery of oligonucleotide-based agents to extra-hepatic (i.e., non-hepatocyte) cells.

While various attempts over the past several years have been made to direct oligonucleotide-based agents to certain extra-hepatic cell types, including skeletal muscle cells, adipocytes, cardiac myocytes, and the like, using, for example, cholesterol conjugates (which is non-specific and has the known disadvantage of distributing to various undesired tissues and organs) and lipid-nanoparticles (LNPs) (which have been frequently reported to have toxicity concerns), to date none have achieved suitable delivery. As a result, there remains a need for a delivery vehicle to direct oligonucleotide-based agents, and RNAi agents in particular, to non-hepatocyte cell types.

### SUMMARY OF THE INVENTION

Disclosed herein are compounds comprising a lipid PK/PD modulator conjugated (or connected) to an oligonucleotide-based agent. Lipid PK/PD modulator precursors are also disclosed herein.

One aspect of the invention provides a compound of Formula (I):

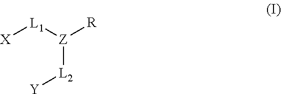

or a pharmaceutically acceptable salt thereof, wherein R is -L_{A}-R_{Z}; L_{A} is a bond or a bivalent moiety connecting R_{Z} to Z; R_{Z} comprises an oligonucleotide-based agent; Z is CH, phenyl or N; L₁ and L₂ are each independently linkers comprising at least about 5 polyethylene glycol (PEG) units; and X and Y are each independently lipids comprising from about 10 to about 50 carbon atoms.

In some embodiments, L₁ and L₂ each independently comprise about 15 to about 100 PEG units. In some embodiments, L₁ and L₂ each independently comprise about 20 to about 60 PEG units. In some embodiments, L₁ and L₂ each independently comprise about 20 to about 30 PEG units. In other embodiments, L₁ and L₂ each independently comprise about 40 to about 60 PEG units. And, in some embodiments, one of L₁ and L₂ comprises about 20 to about 30 PEG units and the other comprises about 40 to about 60 PEG units. In some embodiments, each of L₁ and L₂ is independently selected from the group consisting of the moieties identified in Table 1.

In some embodiments, L_{A} is selected from the group consisting of the moieties identified in Table 4.

In some embodiments, at least one of X and Y is an unsaturated lipid. In some embodiments, at least one of X and Y is a saturated lipid. In some embodiments, at least one of X and Y is a branched lipid. In some embodiments, at least one of X and Y is a lipid comprising from about 10 to about 25 carbon atoms. In some embodiments, at least one of X and Y is cholesteryl. In some embodiments, at least one of X and Y is selected from the group consisting of the moieties identified in Table 3. In some embodiments, each of X and Y are independently selected from the group consisting of the moieties identified in Table 3.

In some embodiments, the oligonucleotide-based agent is an RNAi agent.

Another aspect of the invention provides a compound of Formula (Ia):

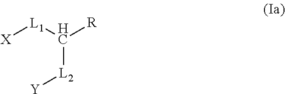

or a pharmaceutically acceptable salt thereof, wherein each of R, L₁, L₂, X, and Y is as defined in any of the embodiments of the compound of Formula (I).

In some embodiments, L₁ and L₂ are independently selected from the group consisting of

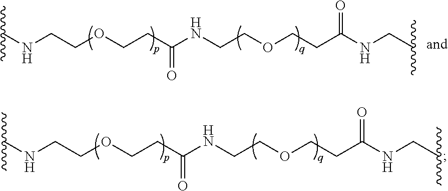

wherein each p is independently 20, 21, 22, 23, 24, or 25; each q is independently 20, 21, 22, 23, 24, or 25; and each [symbol] indicates a point of connection to X, Y, or CH of Formula (Ia).

In some embodiments, L_{A} is

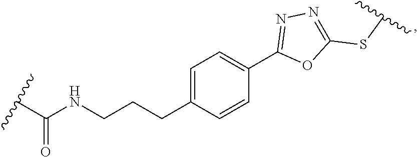 [SMILES: *Sc1nnc(-c2ccc(CCCNC(*)=O)cc2)o1]

and each [symbol] indicates a point of connection to R_{Z} or CH of Formula (Ia).

In some embodiments each of X and Y are

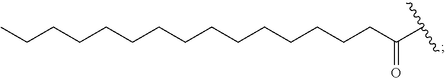 [SMILES: *C(=O)CCCCCCCCCCCCCCC]

and [symbol] indicates a point of connection to L₁ or L₂.

Another aspect of the invention provides a compound of Formula (Ib):

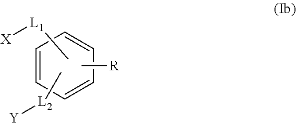

or a pharmaceutically acceptable salt thereof, wherein each of R, L₁, L₂, X, and Y are as defined in any of the embodiments of the compound of Formula (I) or (Ia).

Another aspect of the present invention provides a compound of Formula (Ib1):

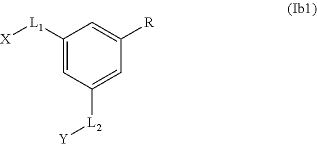

or a pharmaceutically acceptable salt thereof, wherein R, L₁, L₂, X, and Y are as defined in any of the embodiments of the compound of Formula (I), (Ia), or (Ib).

Another aspect of the invention provides a compound of Formula (Ic):

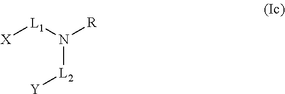

or a pharmaceutically acceptable salt thereof, wherein R, L₁, L₂, X, and Y are as defined in any of the embodiments of the compound of Formula (I), (Ia), (Ib), or (Ib1).

Another aspect of the present invention provides a compound of Formula (Id):

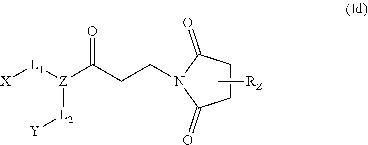

or a pharmaceutically acceptable salt thereof, wherein R_{Z}, Z, L₁, L₂, X, and Y are as defined in any of the embodiments of the compound of Formula (I), (Ia), (Ib) (Ib1), or (Ic).

Another aspect of the invention provides a compound of Formula (II):

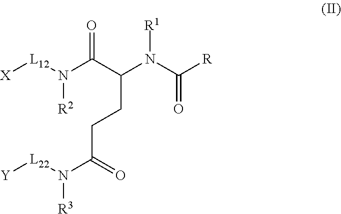

or a pharmaceutically acceptable salt thereof, wherein R, X, and Y are as defined for any embodiments of the compound of Formula (I), (Ia), (Ib), (Ib1), or (Ic); L₁₂ is L₁ as defined for any embodiments of the compound of Formula (I), (Ia), (Ib), (Ib1), (Ic), or (Id); L₂₂ is L₂ as defined for any embodiments of the compound of Formula (I), (Ia), (Ib), (Ib1), (Ic), or (Id); and R¹, R² and R³ are each independently hydrogen or C₁₋₆ alkyl.

In some embodiments, R is L_{A2}-R_{Z}; L_{A2} is a bond or a bivalent moiety connecting R_{Z} to —C(O)—; R_{Z} comprises an oligonucleotide-based agent; R¹, R², and R³ are each independently hydrogen or C₁₋₆ alkyl; L₁₂ and L₂₂ are each independently linkers comprising at least about 5 PEG units; and X and Y are each independently lipids comprising from about 10 to about 50 carbon atoms.

In some embodiments, L₁₂ and L₂₂ each independently comprise about 15 to about 100 PEG units. In some embodiments, L₁₂ and L₂₂ each independently comprise about 20 to about 60 PEG units. In some embodiments, L₁₂ and L₂₂ each independently comprise about 20 to about 30 PEG units. In other embodiments, L₁₂ and L₂₂ each independently comprise about 40 to about 60 PEG units. And, in some embodiments, one of L₁₂ and L₂₂ comprises about 20 to about 30 PEG units and the other comprises about 40 to about 60 PEG units. In some embodiments, each of L₁₂ and L₂₂ is independently selected from the group consisting of the moieties identified in Table 5.

In some embodiments, at least one of X and Y is an unsaturated lipid. In some embodiments, at least one of X and Y is a saturated lipid. In some embodiments, at least one of X and Y is a branched lipid. In some embodiments, at least one of X and Y is a lipid comprising from about 10 to about 25 carbon atoms. In some embodiments, at least one of X and Y is cholesteryl. In some embodiments, at least one of X and Y is selected from the group consisting of the moieties identified in Table 6. In some embodiments, each of X and Y is independently selected from the group consisting of the moieties identified in Table 6.

In some embodiments, L_{A2} is selected from the group consisting of the moieties identified in Table 7.

In some embodiments, each of R¹, R² and R³ is independently hydrogen or C₁₋₃ alkyl. In some embodiments, each of R¹, R² and R³ is hydrogen.

In some embodiments, the oligonucleotide-based agent is an RNAi agent.

Another aspect of the invention provides a compound of Formula (III):

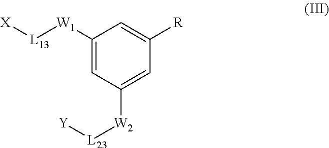

or a pharmaceutically acceptable salt thereof, wherein R, X, and Y are as defined for any embodiments of the compound of Formula (I), (Ia), (Ib), (Ib1), (Ic), or (II); L₁₃ is L₁ as defined for any embodiments of the compound of Formula (I), (Ia), (Ib), (Ib1), (Ic), or (Id), or L₁₃ is L₁₂ as defined for any embodiments of the compound of Formula (II); L₂₃ is L₂ as defined for any embodiments of the compound of Formula (I), (Ia), (Ib), (Ib1), (Ic), or (Id), or L₂₃ is L₂₂ as defined for any embodiments of the compound of Formula (II); W₁ is —C(O)NR₁— or —OCH₂CH₂NR₁C(O)—, wherein R₁ is hydrogen or C₁₋₆ alkyl; and W₂ is —C(O)NR₂— or —OCH₂CH₂NR₂C(O)—, wherein R₂ is hydrogen or C₁₋₆ alkyl.

In some embodiments, R is L_{A3}-R_{Z}; L_{A3} is a bond or a bivalent moiety connecting R_{Z} to the phenyl ring; R_{Z} comprises an oligonucleotide-based agent; W₁ is —C(O)NR₁— or —OCH₂CH₂NR₁C(O)—, wherein R₁ is hydrogen or C₁₋₆ alkyl; W₂ is —C(O)NR₂— or —OCH₂CH₂NR₂C(O)—, wherein R₂ is hydrogen or C₁₋₆ alkyl; L₁₃ and L₂₃ are each independently linkers comprising at least about 5 PEG units; and X and Y are each independently lipids comprising from about 10 to about 50 carbon atoms.

In some embodiments, L₁₃ and L₂₃ each independently comprise about 15 to about 100 PEG units. In some embodiments, L₁₃ and L₂₃ each independently comprise about 20 to about 60 PEG units. In some embodiments, L₁₃ and L₂₃ each independently comprise about 20 to about 30 PEG units. In other embodiments, L₁₃ and L₂₃ each independently comprise about 40 to about 60 PEG units. And, in some embodiments, one of L₁₃ and L₂₃ comprises about 20 to about 30 PEG units and the other comprises about 40 to about 60 PEG units. In some embodiments, each of L₁₃ and L₂₃ is independently selected from the group consisting of the moieties identified in Table 8.

In some embodiments, at least one of X and Y is an unsaturated lipid. In some embodiments, at least one of X and Y is a saturated lipid. In some embodiments, at least one of X and Y is a branched lipid. In some embodiments, at least one of X and Y is a lipid comprising from about 10 to about 25 carbon atoms. In some embodiments, at least one of X and Y is cholesteryl. In some embodiments, at least one of X and Y is selected from the group consisting of the moieties identified in Table 9. In some embodiments, each of X and Y is independently selected from the group consisting of the moieties identified in Table 9.

In some embodiments, L_{A3} is selected from the group consisting of the moieties identified in Table 10.

In some embodiments, each of R¹ and R² is independently hydrogen or C₁₋₃ alkyl. In some embodiments, each of R¹ and R² is hydrogen.

In some embodiments, the oligonucleotide-based agent is an RNAi agent.

Another aspect of the present invention provides a compound of Formula (IIIa):

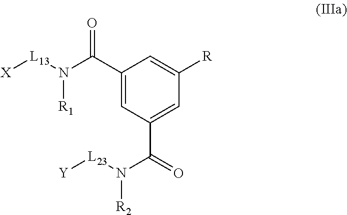

or a pharmaceutically acceptable salt thereof, wherein each of R, X, and Y is as defined for any embodiments of the compound of Formula (I), (Ia), (Ib), (Ib1), (Ic), (II), or (III); L₁₃ is L₁ as defined for any embodiments of the compound of Formula (I), (Ia), (Ib), (Ib1), (Ic), or (Id), L₁₃ is L₁₂ as defined for any embodiments of the compound of Formula (II), or L₁₃ is as defined in any embodiments of the compound of Formula (III); L₂₃ is L₂ as defined for any embodiments of the compound of Formula (I), (Ia), (Ib), (Ib1), (Ic), or (Id), L₂₃ is L₂₂ as defined for any embodiments of the compound of Formula (II), or L₁₃ is as defined in any embodiments of the compound of Formula (III); and each of R₁ and R₂ are as defined in any embodiments of the compound of Formula (II) or (III).

In some embodiments, R is L_{A3}-R_{Z}; L_{A3} is a bond or a bivalent moiety connecting R_{Z} to the phenyl ring; R_{Z} comprises an oligonucleotide-based agent; R₁ and R₂ are each independently hydrogen or C₁₋₆ alkyl (e.g., methyl, ethyl, n-propyl, n-butyl, or n-pentyl); L₁₃ and L₂₃ are each independently linkers comprising at least about 5 PEG units; and X and Y are each independently lipids comprising from about 10 to about 50 carbon atoms.

In some embodiments, each of L₁₃ and L₂₃ is selected from the group consisting of Linker 1-3 and Linker 2-3 as set forth in Table 8.

In some embodiments, at least one of X and Y is selected from the group consisting of Lipid 3 and Lipid 19 as set forth in Table 9. In some embodiments, each of X and Y is independently selected from the group consisting of Lipid 3 and Lipid 19 as set forth in Table 9.

In some embodiments, L_{A3} is selected from the group consisting of Tether 1-3, Tether 2-3, and Tether 5-3 as set forth in Table 10.

In some embodiments, each of R¹ and R² is independently hydrogen or C₁₋₃ alkyl. In some embodiments, each of R¹ and R² is hydrogen.

In some embodiments, the oligonucleotide-based agent is an RNAi agent.

Another aspect of the present invention provides a compound of Formula (IIIb):

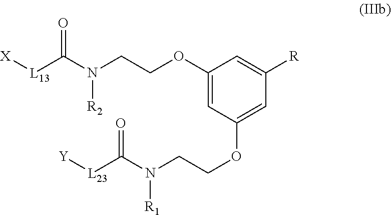

or a pharmaceutically acceptable salt thereof, wherein R, X, and Y are as defined for any embodiments of the compound of Formula (I), (Ia), (Ib), (Ib1), (Ic), (II), (III), or (IIIa); L₁₃ is L₁ as defined for any embodiments of the compound of Formula (I), (Ia), (Ib), (Ib1), (Ic), or (Id), L₁₃ is L₁₂ as defined for any embodiments of the compound of Formula (II), or L₁₃ is as defined in any embodiments of the compound of Formula (III) or (IIIa); L₂₃ is L₂ as defined for any embodiments of the compound of Formula (I), (Ia), (Ib), (Ib1), (Ic), or (Id), L₂₃ is L₂₂ as defined for any embodiments of the compound of Formula (II), or L₂₃ is as defined in any embodiments of the compound of Formula (III) or (IIIa); and each of R₁ and R₂ are as defined in any embodiments of the compound of Formula (II), (III), or (IIIa).

In some embodiments, R is L_{A3}-R_{Z}; L_{A3} is a bond or a bivalent moiety connecting R_{Z} to the phenyl ring; R_{Z} comprises an oligonucleotide-based agent; R₁ and R₂ are each independently selected from hydrogen or C₁₋₆ alkyl; L₁₃ and L₂₃ are each independently linkers comprising at least about 5 PEG units; and X and Y are each independently lipids comprising from about 10 to about 50 carbon atoms.

In some embodiments, each of L₁₃ and L₂₃ is Linker 3-3 as set forth in Table 8.

In some embodiments, each of X and Y is Lipid 3 as set forth in Table 9.

In some embodiments, L_{A3} is selected from the group consisting of Tether 3-3 and Tether 4-3 as set forth in Table 10.

In some embodiments, each of R¹ and R² is independently hydrogen or C₁₋₃ alkyl. In some embodiments, each of R¹ and R² is hydrogen.

In some embodiments, the oligonucleotide-based agent is an RNAi agent.

Another aspect of the invention provides a compound of Formula (IV):

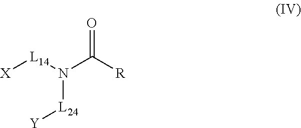

or a pharmaceutically acceptable salt thereof, wherein R, X, and Y are as defined for any embodiments of the compound of Formula (I), (Ia), (Ib), (Ib1), (Ic), (II), (III), (IIIa), or (IIIb); L₁₄ is L₁ as defined for any embodiments of the compound of Formula (I), (Ia), (Ib), (Ib1), or (Ic), L₁₄ is L₁₂ as defined for any embodiments of the compound of Formula (II), or L₁₄ is L₁₃ as defined in any embodiments of the compound of Formula (III), (IIIa), or (IIIb); L₂₄ is L₂ as defined for any embodiments of the compound of Formula (I), (Ia), (Ib), (Ib1), or (Ic), L₂₄ is L₂₂ as defined for any embodiments of the compound of Formula (II), or L₂₄ is L₂₃ as defined in any embodiments of the compound of Formula (III), (IIIa), or (IIIb).

In some embodiments, R is L_{A4}-R_{Z}; L_{A4} is a bond or a bivalent moiety connecting R_{Z} to —C(O)—; R_{Z} comprises an oligonucleotide-based agent; L₁₄ and L₂₄ are each independently linkers comprising at least about 5 PEG units; and X and Y are each independently lipids comprising from about 10 to about 50 carbon atoms.

In some embodiments, L₁₄ and L₂₄ each independently comprise about 15 to about 100 PEG units. In some embodiments, L₁₄ and L₂₄ each independently comprise about 20 to about 60 PEG units. In some embodiments, L₁₄ and L₂₄ each independently comprise about 20 to about 30 PEG units. In other embodiments, L₁₄ and L₂₄ each independently comprise about 40 to about 60 PEG units. And, in some embodiments, one of L₁₄ and L₂₄ comprises about 20 to about 30 PEG units and the other comprises about 40 to about 60 PEG units. In some embodiments, each of L₁₄ and L₂₄ is independently selected from the group consisting of the moieties identified in Table 11.

In some embodiments, at least one of X and Y is an unsaturated lipid. In some embodiments, at least one of X and Y is a saturated lipid. In some embodiments, at least one of X and Y is a branched lipid. In some embodiments, at least one of X and Y is a lipid comprising from about 10 to about 25 carbon atoms. In some embodiments, at least one of X and Y is cholesteryl. In some embodiments, at least one of X and Y is selected from the group consisting of the moieties identified in Table 12. In some embodiments, each of X and Y is independently selected from the group consisting of the moieties identified in Table 12.

In some embodiments, L_{A4} is selected from the group consisting of the moieties identified in Table 13.

In some embodiments, the oligonucleotide-based agent is an RNAi agent.

Another aspect of the invention provides a compound of Formula (IVa):

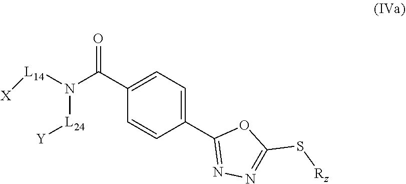

or a pharmaceutically acceptable salt thereof, wherein X and Y are as defined for any embodiments of the compound of Formula (I), (Ia), (Ib), (Ib1), (Ic), (II), (III), (IIIa), (IIIb), or (IV); L₁₄ and L₂₄ are as defined in any of the embodiments of the compound of Formula (IV); and R_{Z} comprises an oligonucleotide-based agent.

In some embodiments, R_{Z} comprises an oligonucleotide-based agent; each of L₁₄ and L₂₄ is independently selected from the group consisting of

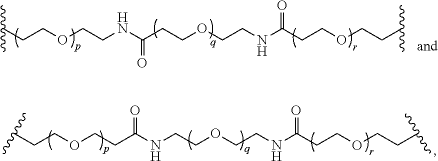

wherein each [symbol] indicates a point of connection to X, Y, or

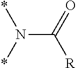

of Formula (IVa), each * indicates the point of attachment to L₁₄ or L₂₄, each p is independently 20, 21, 22, 23, 24, or 25, each q is independently 20, 21, 22, 23, 24, or 25, and each r is independently 2, 3, 4, 5, or 6; and each of X and Y is independently selected from the group consisting of

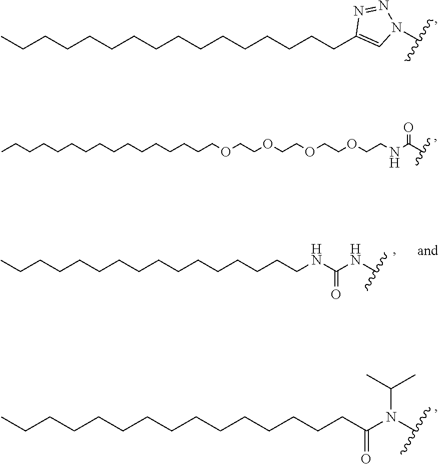 [SMILES: *n1cc(CCCCCCCCCCCCCCCC)nn1 ; *C(=O)NCCOCCOCCOCCOCCCCCCCCCCCCCCCC ; *NC(=O)NCCCCCCCCCCCCCCCC ; *N(C(=O)CCCCCCCCCCCCCCC)C(C)C]

wherein [symbol] indicates a point of connection to L₁₄ or L₂₄.

In another aspect of the invention, a compound is selected from the group consisting of the compounds identified in Table 14, or a pharmaceutically acceptable salt thereof. In another aspect of the invention, a compound is selected from the group consisting of the compounds identified in Table 16, or a pharmaceutically acceptable salt thereof.

Another aspect of the invention provides a compound of Formula (V):

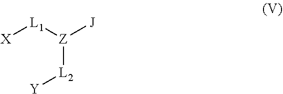

or a pharmaceutically acceptable salt thereof, wherein Z, L₁, L₂, X, and Y are as defined for any embodiments of the compound of Formula (I), (Ia), (Ib), (Ib1), or (Ic); J is L_{A5}-R_{X}; L_{A5} is a bond or a bivalent moiety connecting R_{X} to Z: and R_{X} is a reactive moiety for conjugation with an oligonucleotide-based agent.

In some embodiments, J is L_{A5}-R_{X}; L_{A5} is a bond or a bivalent moiety connecting R_{X} to Z; R_{X} is a reactive moiety for conjugation with an oligonucleotide-based agent; Z is CH, phenyl, or N; L₁ and L₂ are each independently linkers comprising at least about 5 PEG units; and X and Y are each independently lipids comprising from about 10 to about 50 carbon atoms.

In some embodiments, R_{X} is selected from the group consisting of

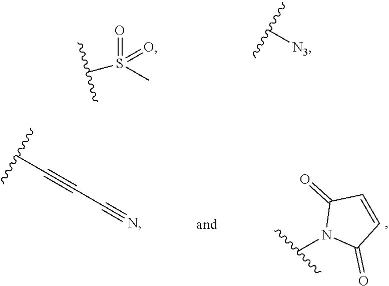

wherein [symbol] indicates a point of connection to L_{A5}.

In some embodiments, L_{A5} is selected from the group consisting of the moieties identified in Table 18.

In another aspect of the invention, a compound is selected from the group consisting of the compounds identified in Table 20, or a pharmaceutically acceptable salt thereof.

Also disclosed herein are methods of making compounds of Formula (I). One aspect of the invention provides a method for making a compound of Formula (I), wherein the method comprises conjugating an oligonucleotide-based agent comprising a first reactive moiety with a compound comprising a lipid and a second reactive moiety to form a compound of Formula (I). In some implementations, the first reactive moiety is selected from the group consisting of a disulfide and a propargyl group. In some implementations, the second reactive moiety is selected from the group consisting of maleimide, sulfone, azide, and alkyne.

Another aspect of the present invention provides a pharmaceutical composition comprising a compound of any of Formulae (I), (Ia), (Ib), (Ib1), (Ic), (Id), (II), (III), (IIIa), (IIIb), (IV), or (IVa), or a pharmaceutically acceptable salt of any of these compounds, and a pharmaceutically acceptable excipient.

Another aspect of the present invention provides a method reducing a target gene expression in vivo, comprising introducing to a cell the compound of any of Formulae (I), (Ia), (Ib), (Ib1), (Ic), (Id), (II), (III), (IIIa), (IIIb), (IV), or (IVa), or a pharmaceutically acceptable salt of any of these compounds, wherein the compound comprises an RNAi agent at least substantially complementary to the target gene.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### DETAILED DESCRIPTION

Lipid PK/PD Modulators

Described herein are compounds comprising PK/PD modulators conjugated to oligonucleotide-based agent(s) to provide delivery of payloads, such as RNA interference (RNAi) agents, to cells in vivo. Without being bound to any particular theory, it is believed that the compounds described herein modulate the pharmacokinetic and or pharmacodynamic properties of corresponding delivery vehicles, thereby increasing the RNAi-induced knockdown of the target gene in a cell. The compounds described herein may facilitate delivery to certain cell types, including but not limited to skeletal muscle cells and adipocytes.

The present invention provides a compound of Formula (I):

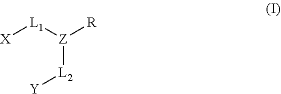

or a pharmaceutically acceptable salt thereof, wherein R is L_{A}-R_{Z}; L_{A} is a bond or a bivalent moiety connecting R_{Z} to Z; R_{Z} comprises an oligonucleotide-based agent (e.g., an RNAi agent); Z is CH, phenyl, or N; L₁ and L₂ are each independently linkers comprising at least about 5 polyethylene glycol (PEG) units; and X and Y are each independently lipids comprising from about 10 to about 50 carbon atoms.

As used herein and as would be understood by one skilled in the art, a polyethylene glycol (PEG) unit refers to repeating units of the formula —(CH₂CH₂O)—. It will be appreciated that, in the chemical structures disclosed herein, PEG units may be depicted as —(CH₂CH₂O)—, —(OCH₂CH₂)—, or —(CH₂OCH₂)—. It will also be appreciated that a numeral indicating the number of repeating PEG units may be placed on either side of the parentheses depicting the PEG units.

In some embodiments, L₁ and L₂ each independently comprise about 15 to about 100 PEG units. In some embodiments, L₁ and L₂ each independently comprise about 20 to about 60 PEG units. In some embodiments, L₁ and L₂ each independently comprise about 20 to about 30 PEG units. In some embodiments, L₁ and L₂ each independently comprise about 40 to about 60 PEG units. In some embodiments, one of L₁ and L₂ comprises about 20 to about 30 PEG units, and the other comprises about 40 to about 60 PEG units. For example, L₁ and L₂ may each independently comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 PEG units. In some embodiments, each of L₁ and L₂ comprise one or more additional bivalent moieties (e.g., —C(O)—, —N(H)—, —N(H)—C(O)—, —C(O)—N(H)—, —S(O)₂—, —S—, and other bivalent moieties that are not PEG) that connect two PEG units in the linker. For instance, each of L₁ and L₂ comprise the structure

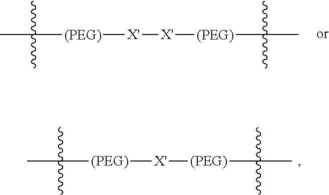

wherein each X′ is independently a bivalent moiety other than a PEG unit, and each PEG is a PEG unit.

In some embodiments, each of L₁ and L₂ is independently selected from the group consisting of the moieties identified in Table 1.

TABLE 1
Example L₁ and L₂ moieties of the present invention.

| Name | Structure |
|---|---|
| Linker 1 | 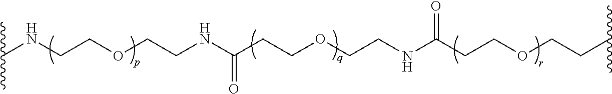 |
| Linker 2 | 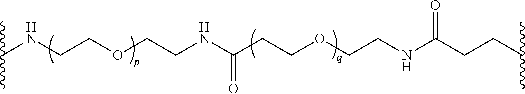 |
| Linker 3 | 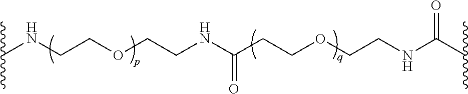 |
| Linker 4 | 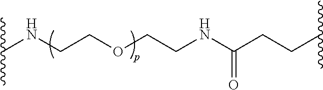 |
| Linker 5 | 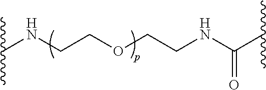 |
| Linker 6 | 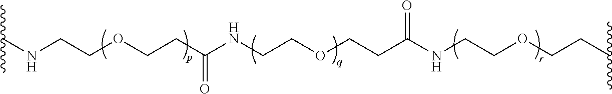 |
| Linker 7 | 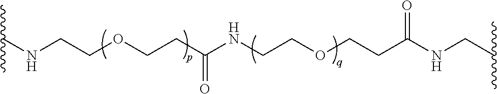 |
| Linker 8 | 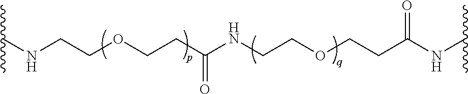 |
| Linker 9 | 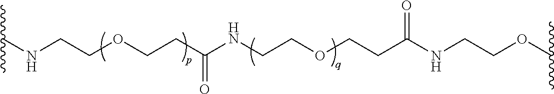 |
| Linker 10 | 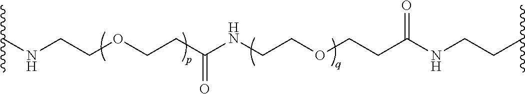 |
| Linker 11 | 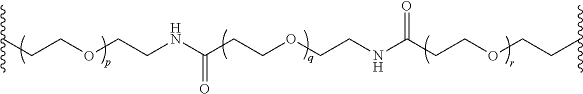 |
| Linker 12 | 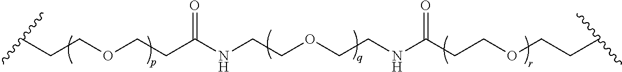 |

Wherein, each p is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; each q is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; each r is independently 1, 2, 3, 4, 5, 6, 7, 8, 9, or 10; and each [symbol] indicates a point of connection to X, Y, or Z, provided that:
(i) in Linker 1, 6, and 11, p+q+r≥5;
(ii) in Linker 2, 3, 7, 8, 9, and 10, p+q≥5; and
(iii) in Linker 4 and 5 p≥5.

In some embodiments, each p is independently 20, 21, 22, 23, 24, or 25; each q is independently 20, 21, 22, 23, 24, or 25; and each r is independently 2, 3, 4, 5, or 6. In some embodiments, each p is independently 23 or 24. In some embodiments, each q is independently 23 or 24. In some embodiments, each r is 4.

In some embodiments, each of L₁ and L₂ is independently selected from the group consisting of the moieties identified in Table 2.

TABLE 2
Example L₁ and L₂ moieties of the present invention.
Structure

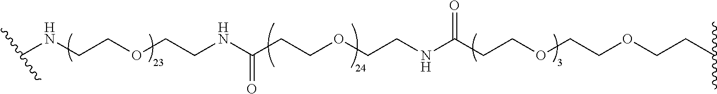

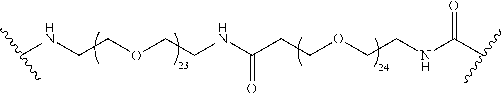

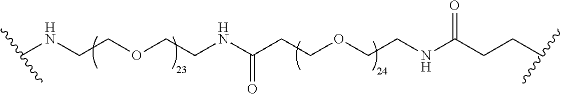

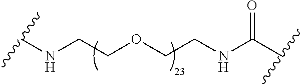

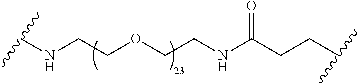

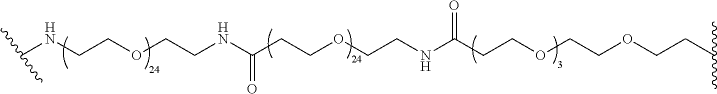

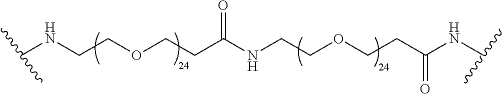

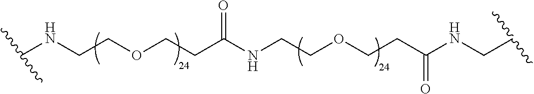

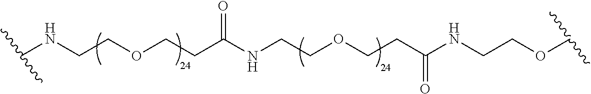

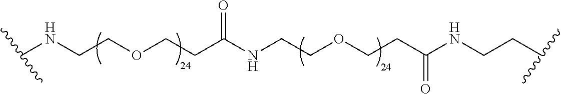

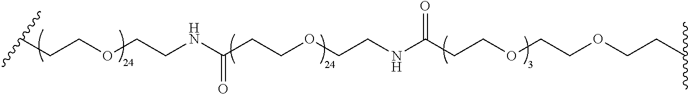

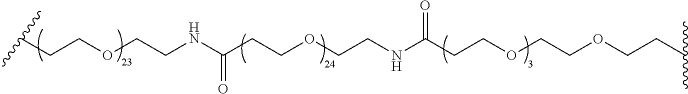

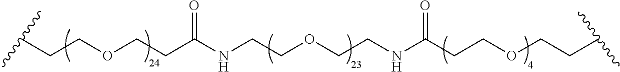

wherein [symbol] indicates a point of connection to X, Y, or Z.

In some embodiments, L₁ and L₂ are the same. In other embodiments, L₁ and L₂ are different.

In some embodiments, at least one of X and Y is an unsaturated lipid. In some embodiments, each of X and Y is an unsaturated lipid. In some embodiments, at least one of X and Y is a saturated lipid. In some embodiments, each of X and Y is a saturated lipid. In some embodiments, at least one of X and Y is a branched lipid. In some embodiments, each of X and Y is a branched lipid. In some embodiments, at least one of X and Y is a straight chain lipid. In some embodiments, each of X and Y is a straight chain lipid. In some embodiments, at least one of X and Y is cholesteryl. In some embodiments, each of X and Y is cholesteryl. In some embodiments, X and Y are the same. In other embodiments, X and Y are different.

In some embodiments, at least one of X and Y comprises from about 10 to about 45 carbon atoms. In some embodiments, at least one of X and Y comprises from about 10 to about 40 carbon atoms. In some embodiments, at least one of X and Y comprises from about 10 to about 35 carbon atoms. In some embodiments, at least one of X and Y comprises from about 10 to about 30 carbon atoms. In some embodiments, at least one of X comprises from about 10 to about 25 carbon atoms. In some embodiments, at least one of X and Y comprises from about 10 to about 20 carbon atoms.

In some embodiments, X and Y each independently comprise from about 10 to about 45 carbon atoms. In some embodiments, X and Y each independently comprise from about 10 to about 40 carbon atoms. In some embodiments, X and Y each independently comprise from about 10 to about 35 carbon atoms. In some embodiments, X and Y each independently comprise from about 10 to about 30 carbon atoms. In some embodiments, X and Y each independently comprise from about 10 to about 25 carbon atoms. In some embodiments, X and Y each independently comprise from about 10 to about 20 carbon atoms. For example, X and Y may each independently comprise 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 carbon atoms.

In some embodiments, at least one of X and Y is selected from the group consisting of the moieties identified in Table 3. In some embodiments, each of X and Y are independently selected from the group consisting of the moieties identified in Table 3.

TABLE 3
Example X and Y moieties of the present invention.

| Name | Structure |
|---|---|
| Lipid 1 | 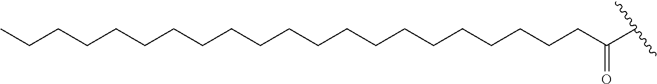 [SMILES: *C(=O)CCCCCCCCCCCCCCCCCCCCC] |
| Lipid 2 | 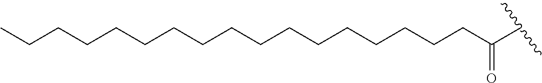 [SMILES: *C(=O)CCCCCCCCCCCCCCCCC] |
| Lipid 3 | 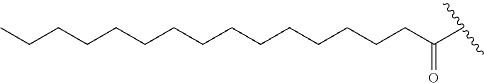 [SMILES: *C(=O)CCCCCCCCCCCCCCC] |
| Lipid 4 (Cholesteryl) | 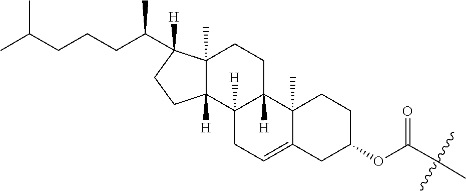 |
| Lipid 5 | 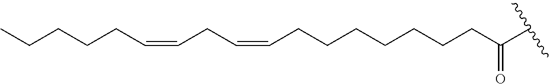 [SMILES: *C(=O)CCCCCCC/C=C\C/C=C\CCCCC] |
| Lipid 6 | 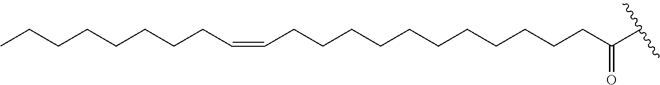 [SMILES: *C(=O)CCCCCCCCCCC/C=C\CCCCCCCC] |
| Lipid 7 | 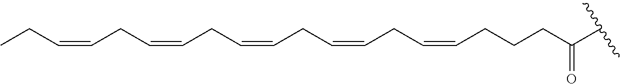 [SMILES: *C(=O)CCC/C=C\C/C=C\C/C=C\C/C=C\C/C=C\CC] |
| Lipid 8 | 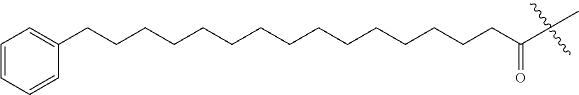 [SMILES: *C(=O)CCCCCCCCCCCCCCCc1ccccc1] |
| Lipid 9 | 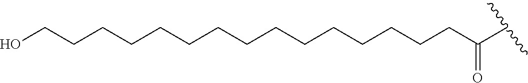 [SMILES: *C(=O)CCCCCCCCCCCCCCCO] |
| Lipid 10 | 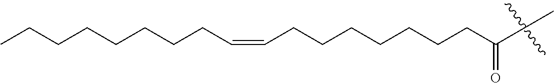 [SMILES: *C(=O)CCCCCCC/C=C\CCCCCCCC] |
| Lipid 11 | 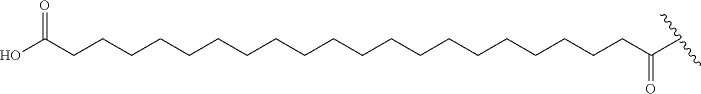 [SMILES: *C(=O)CCCCCCCCCCCCCCCCCCCCC(=O)O] |
| Lipid 12 | 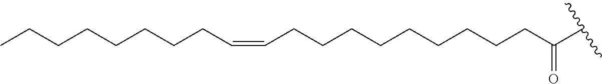 [SMILES: *C(=O)CCCCCCCCC/C=C\CCCCCCCC] |
| Lipid 14 | 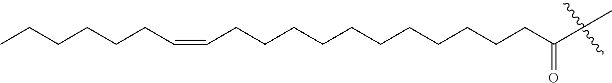 [SMILES: *C(=O)CCCCCCCCCCC/C=C\CCCCCC] |
| Lipid 15 | 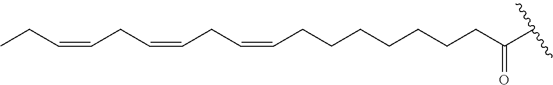 [SMILES: *C(=O)CCCCCCC/C=C\C/C=C\C/C=C\CC] |
| Lipid 16 | 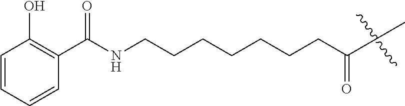 [SMILES: *C(=O)CCCCCCCNC(=O)c1ccccc1O] |
| Lipid 17 | 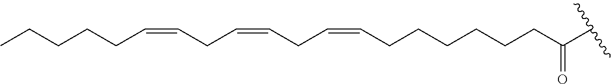 [SMILES: *C(=O)CCCCCC/C=C\C/C=C\C/C=C\CCCCC] |
| Lipid 18 | 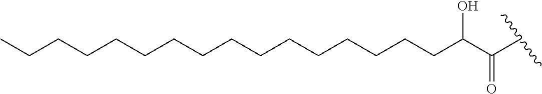 [SMILES: *C(=O)C(O)CCCCCCCCCCCCCCCC] |
| Lipid 19 | 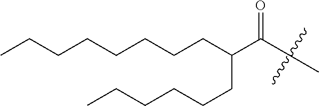 [SMILES: *C(=O)C(CCCCCC)CCCCCCCC] |
| Lipid 20 | 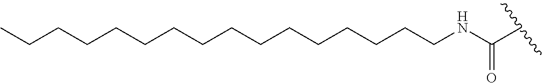 [SMILES: *C(=O)NCCCCCCCCCCCCCCCC] |
| Lipid 21 | 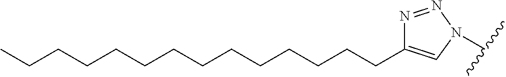 [SMILES: *n1cc(CCCCCCCCCCCCCC)nn1] |
| Lipid 22 | 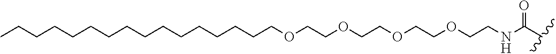 [SMILES: *C(=O)NCCOCCOCCOCCOCCCCCCCCCCCCCCCC] |
| Lipid 23 | 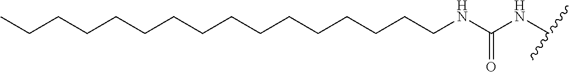 [SMILES: *NC(=O)NCCCCCCCCCCCCCCCC] |
| Lipid 24 | 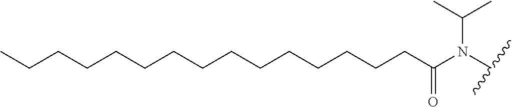 [SMILES: *N(C(=O)CCCCCCCCCCCCCCC)C(C)C] |

wherein [symbol] indicates a point of connection to L₁ or L₂.

In some embodiments, L_{A} comprises at least one PEG unit. In some embodiments, L_{A} is free of any PEG units. In some embodiments, L_{A} comprises —C(O)—, —C(O)N(H)—, —N(H)C(O)—, optionally substituted alkoxy, or an optionally substituted alkyleneheterocyclyl. In some embodiments, L_{A} is a bond.

In some embodiments, L_{A} is selected from the group consisting of the moieties identified in Table 4.

TABLE 4
Example L_{A} moieties of the present invention.

| Name | Structure |
|---|---|
| Tether 1 | 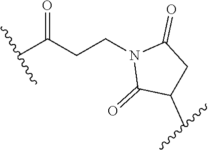 [SMILES: *C(=O)CCN1C(=O)CC(*)C1=O] |
| Tether 2 | 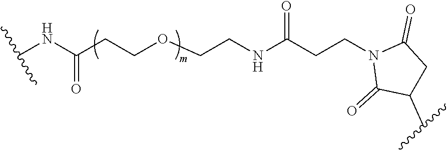 |
| Tether 3 | 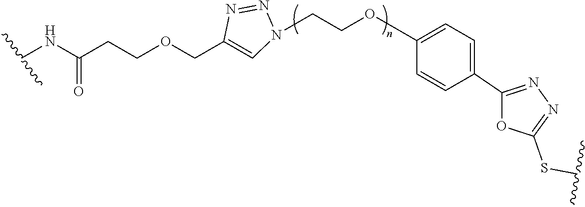 |
| Tether 4 | 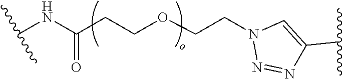 |
| Tether 5 | 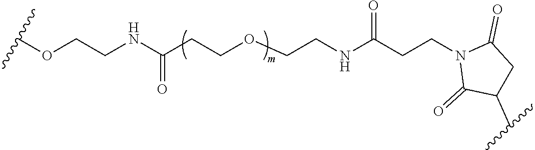 |
| Tether 6 | 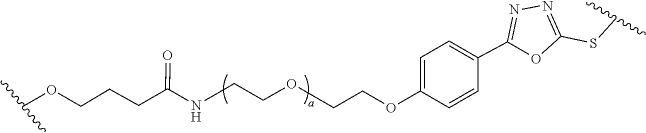 |
| Tether 7 | 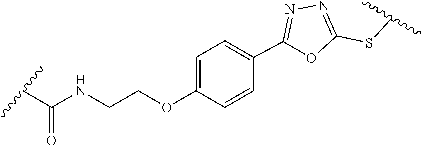 [SMILES: *Sc1nnc(-c2ccc(OCCNC(*)=O)cc2)o1] |
| Tether 8 | 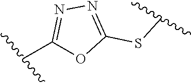 [SMILES: *Sc1nnc(*)o1] |
| Tether 9 | 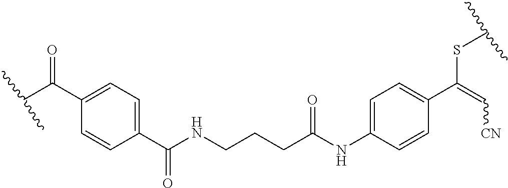 [SMILES: *SC(=CC#N)c1ccc(NC(=O)CCCNC(=O)c2ccc(C(*)=O)cc2)cc1] |
| Tether 10 | 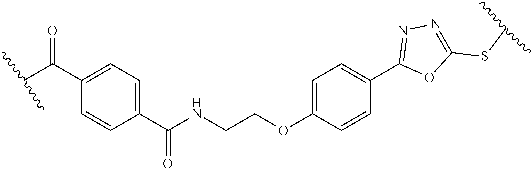 [SMILES: *Sc1nnc(-c2ccc(OCCNC(=O)c3ccc(C(*)=O)cc3)cc2)o1] |
| Tether 11 | 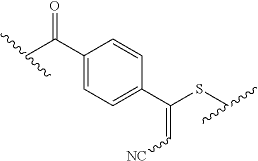 [SMILES: *SC(=CC#N)c1ccc(C(*)=O)cc1] |
| Tether 12 | 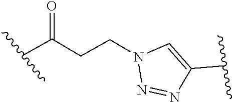 [SMILES: *C(=O)CCn1cc(*)nn1] |
| Tether 13 | 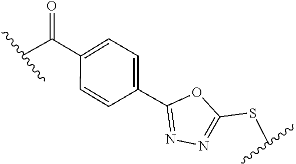 [SMILES: *Sc1nnc(-c2ccc(C(*)=O)cc2)o1] |
| Tether 14 | 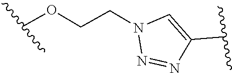 [SMILES: *OCCn1cc(*)nn1] |

wherein, each of m, n, o, and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to Z or R_{Z}.

In some embodiments, each m is independently 1, 2, 3, 4, 5, 6, 7, 8, 9, 10, 20, 21, 22, 23, or 25; each n is independently 2, 3, 4, or 5; each a is independently 2, 3, or 4; and each o is independently 2, 3, 4, 5, 6, 7, 8, 9, 10, 11, 12, or 13. In some embodiments, each m is independently 2, 4, 8, or 24. In some embodiments, each n is 3. In some embodiments, each o is independently 4, 8, or 12. In some embodiments, each a is 3.

In some embodiments, the oligonucleotide-based agent is an RNAi agent as described herein.

Another aspect of the present invention provides a compound of Formula (Ia):

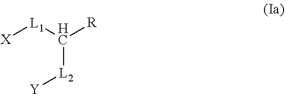

or a pharmaceutically acceptable salt thereof, wherein each of R, L₁, L₂, X, and Y is as defined in any of the embodiments of the compound of Formula (I).

In some embodiments, X and Y are each independently selected from the group consisting of Lipid 3, Lipid 4, Lipid, 5, Lipid 6, Lipid 7, Lipid 10, Lipid 12, and Lipid 19 as set forth in Table 3, wherein each [symbol] indicates a point of connection to L₁ or L₂.

In some embodiments, each of L₁ and L₂ is independently selected from the group consisting of Linker 2, Linker 3, Linker 4, and Linker 5 as set forth in Table 1, wherein each [symbol] indicates a point of connection to X, Y, or CH of Formula (Ia). In some embodiments, each p is 23. In some embodiments, each q is 24.

In some embodiments, L_{A} is selected from the group consisting of Tether 2, Tether 3, and Tether 4 as set forth in Table 4. In some embodiments, each m is independently 2, 4, 8, or 24. In some embodiments, each n is 4. In some embodiments, each o is independently 4, 8, or 12.

In some embodiments L₁ and L₂ are independently selected from the group consisting of

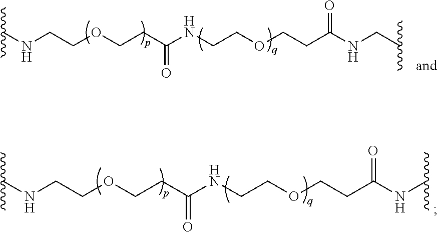

wherein, each p is independently 20, 21, 22, 23, 24, or 25; each q is independently 20, 21, 22, 23, 24, or 25; and each [symbol] indicates a point of connection to X, Y, or CH of Formula (Ia). In some embodiments, each p is 24. In some embodiments, each q is 24.

In some embodiments, L_{A} is

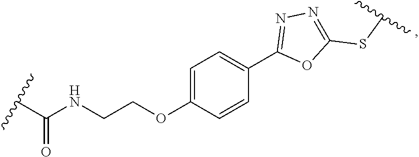 [SMILES: *Sc1nnc(-c2ccc(OCCNC(*)=O)cc2)o1]

and each [symbol] indicates a point of connection to R_{Z} or CH of Formula (Ia).

In some embodiments, each of X and Y are

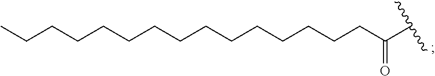 [SMILES: *C(=O)CCCCCCCCCCCCCCC]

wherein [symbol] indicates a point of connection to L₁ or L₂.

In some embodiments, the compound of Formula (Ia) is selected from the group consisting of LP 210a or LP 217a as set forth in Table 14, or a pharmaceutically acceptable salt of any one of these compounds, wherein each R is L_{A}-R_{Z}; L_{A} is a bond or a bivalent moiety connecting R_{Z} to the rest of the compound; and R_{Z} comprises an oligonucleotide based agent.

In some embodiments, the compound of Formula (Ia) is selected from the group consisting of LP 210b and LP 217b as set forth in Table 16, or a pharmaceutically acceptable salt of any one of these compounds, wherein each R_{Z} comprises an oligonucleotide based agent.

Another aspect of the present invention provides a compound of Formula (Ib):

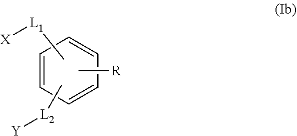

or a pharmaceutically acceptable salt thereof, wherein each of R, L₁, L₂, X, and Y are as defined in any of the embodiments of the compound of Formula (I) or (Ta).

In some embodiments, X and Y are each independently selected from the group consisting of Lipid 3 and Lipid 19 as set forth in Table 3, wherein each [symbol] indicates a point of connection to L₁ or L₂. In some embodiments, X and Y are each Lipid 3. In some embodiments, each of X and Y are each Lipid 19.

In some embodiments, each of L₁ and L₂ is independently selected from the group consisting of Linker 3, Linker 5, and Linker 9 as set forth in Table 1, wherein each [symbol] indicates a point of connection to X, Y, or the phenyl ring of Formula (Ib). In some embodiments, each p is 23 or 24. In some embodiments, each q is 24.

In some embodiments, L_{A} is selected from the group consisting of Tether 5, Tether, 6, Tether 7, Tether 8, and Tether 14 as set forth in Table 4, wherein each [symbol] indicates a point of connection to R_{Z} or the phenyl ring of Formula (Ib). In some embodiments, each m is 2 or 4. In some embodiments, each a is 3.

Another aspect of the present invention provides a compound of Formula (Ib1):

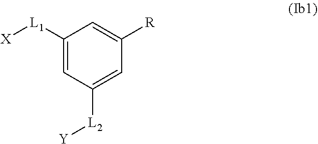

or a pharmaceutically acceptable salt thereof, wherein R, L₁, L₂, X, and Y are as defined in any of the embodiments of the compound of Formula (I), (Ta), or (Tb).

Another aspect of the present invention provides a compound of Formula (Ic):

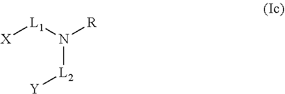

or a pharmaceutically acceptable salt thereof, wherein R, L₁, L₂, X, and Y are as defined in any of the embodiments of the compound of Formula (I), (Ia), (Ib), or (Ib1).

In some embodiments, X and Y are each independently selected from the group consisting of Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, and Lipid 24 as set forth in Table 3, wherein each [symbol] indicates a point of connection to L₁ and L₂. In some embodiments, each of X and Y is Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, or Lipid 24.

In some embodiments, each of L₁ and L₂ is independently selected from the group consisting of Linker 1, Linker 6, Linker 10, Linker 11, and Linker 12 as set forth in Table 1, wherein each [symbol] indicates a point of connection to X, Y, or N of Formula (Ic). In some embodiments, each p is independently 23 or 24. In some embodiments, each q is independently 23 or 24. In some embodiments, each r is 4.

In some embodiments, L_{A} is selected from the group consisting of Tether 1, Tether 9, [symbol] Tether 10, Tether 11, Tether 12, and Tether 13 as set forth in Table 4, wherein each indicates a point of connection to R_{Z} or N of Formula (Ic).

Another aspect of the present invention provides a compound of Formula (Id):

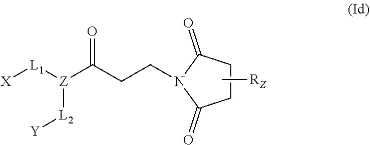

or a pharmaceutically acceptable salt thereof, wherein R_{Z}, Z, L₁, L₂, X, and Y are as defined in any of the embodiments of the compound of Formula (I), (Ia), (Ib) (Ib1), or (Ic).

Another aspect of the present invention provides a compound of Formula (II):

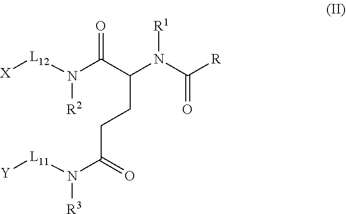

or a pharmaceutically acceptable salt thereof, wherein R, X, and Y are as defined for any embodiments of the compound of Formula (I), (Ia), (Ib), (Ib1), or (Ic); L₁₂ is L₁ as defined for any embodiments of the compound of Formula (I), (Ia), (Ib), (Ib1), (Ic), or (Id); L₂₂ is L₂ as defined for any embodiments of the compound of Formula (I), (Ia), (Ib), (Ib1), (Ic), or (Id); and R¹, R² and R³ are each independently hydrogen or C₁₋₆ alkyl.

In some embodiments; R is L_{A2}-R_{Z}; L_{A2} is a bond or a bivalent moiety connecting R_{Z} to —C(O)—; R_{Z} comprises an oligonucleotide-based agent; R¹, R₂ and R³ are each independently hydrogen or C₁₋₆ alkyl; L₁₂ and L₂₂ are each independently linkers comprising at least about 5 PEG units; and X and Y are each independently lipids comprising from about 10 to about 50 carbon atoms.

In some embodiments, each of L₁₂ and L₂₂ is independently selected from the group consisting of the moieties identified in Table 5.

TABLE 5
Example L₁₂ and L₂₂ moieties of the present invention.

| Name | Structure |
|---|---|
| Linker 1-2 | 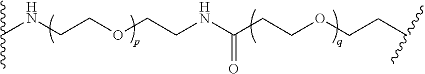 |
| Linker 2-2 | 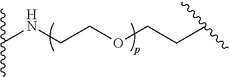 |

wherein, p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and each [symbol] indicates a point of connection to X, Y, —NR²—, or —NR³—, provided that:
(i) in Linker 1-2, p+q≥5; and
(ii) in Linker 2-2, p≥5.

In some embodiments, each p is independently 20, 21, 22, 23, 24, or 25. In some embodiments, each q is 20, 21, 22, 23, 24, or 25. In some embodiments, each p is independently 23 or 24. In some embodiments, each p is 23. In some embodiments, each q is 24.

In some embodiments, L₁₂ and L₂₂ are the same. In other embodiments, L₁₂ and L₂₂ are different.

In some embodiments, at least one of X and Y is selected from the group consisting of the moieties identified in Table 3, wherein each [symbol] indicates a point of connection to L₁₂ or L₂₂. In some embodiments, each of X and Y is independently selected from the group consisting of the moieties identified in Table 3, wherein each [symbol] indicates a point of connection to L₁₂ or L₂₂.

In some embodiments, at least one of X and Y is selected from the group consisting of the moieties identified in Table 6. In some embodiments, each of X and Y is independently selected from the group consisting of the moieties identified in Table 6.

TABLE 6
Example X and Y moieties of the compound of Formula (II).

| Name | Structure |
|---|---|
| Lipid 3 | 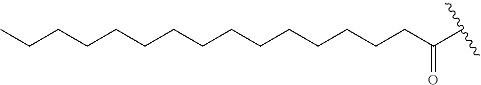 [SMILES: *C(=O)CCCCCCCCCCCCCCC] |
| Lipid 4 | 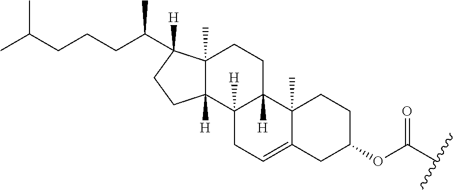 |
| Lipid 5 | 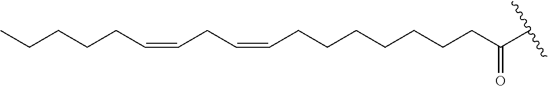 [SMILES: *C(=O)CCCCCCC/C=C\C/C=C\CCCCC] |
| Lipid 6 | 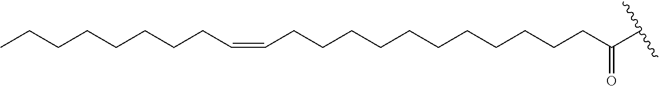 [SMILES: *C(=O)CCCCCCCCCCC/C=C\CCCCCCCC] |
| Lipid 7 | 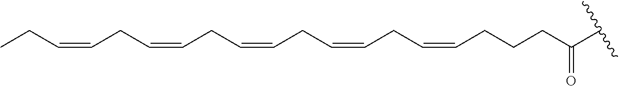 [SMILES: *C(=O)CCC/C=C\C/C=C\C/C=C\C/C=C\C/C=C\CC] |
| Lipid 10 | 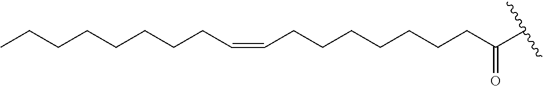 [SMILES: *C(=O)CCCCCCC/C=C\CCCCCCCC] |
| Lipid 12 | 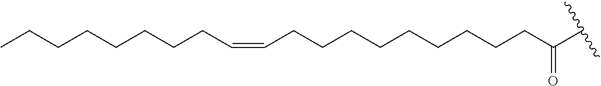 [SMILES: *C(=O)CCCCCCCCC/C=C\CCCCCCCC] |
| Lipid 19 | 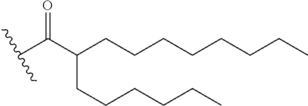 [SMILES: *C(=O)C(CCCCCC)CCCCCCCC] |

wherein [symbol] indicates a point of connection to L₂ or L₂₂.

In some embodiments, L_{A2} comprises at least one PEG unit. In some embodiments, L_{A2} is free of any PEG units. In some embodiments, L_{A2} comprises —C(O)—, —C(O)NH—, optionally substituted alkoxy, or an optionally substituted alkyleneheterocyclyl. In some embodiments, L_{A2} is a bond.

In some embodiments, L_{A2} is selected from the group consisting of the moieties identified in Table 7.

TABLE 7
Example L_{A2} moieties of the present invention.

| Name | Structure |
|---|---|
| Tether 1-2 | 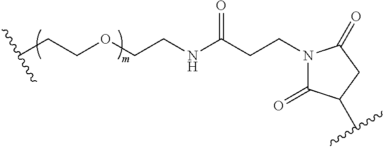 |
| Tether 2-2 | 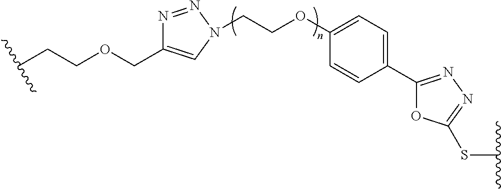 |
| Tether 3-2 | 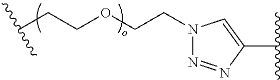 |

wherein each of m, n, and o is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to R_{Z} or —C(O)—.

In some embodiments, m is 1, 2, 3, 4, 5, 6, 7, 8, 9, 10, 20, 21, 22, 23, or 25. In some embodiments, m is 2, 4, 8, or 24. In some embodiments, n is 2, 3, 4, or 5. In some embodiments, n is 4. In some embodiments, o is 2, 3, 4, 5, 6, 7, 8, 9, 10, 11, 12, or 13. In some embodiments, o is 4, 8, or 12.

In some embodiments, each of R¹, R² and R³ is independently hydrogen or C₁₋₃ alkyl. In some embodiments, each of R¹, R² and R³ is hydrogen.

In some embodiments, the compound of Formula (II) is selected from the group consisting of LP 38a, LP 39a, LP 43a, LP 44a, LP 45a, LP 47a, LP 53a, LP 54a, LP 55a, LP 57a, LP 58a, LP 59a, LP 62a, LP 101a, LP 104a, and LP 111a as set forth in Table 14, or a pharmaceutically acceptable salt of any of these compounds, wherein each R is L_{A2}-R_{Z}; L_{A2} is a bond or a bivalent moiety connecting R_{Z} to —C(O)—; and R_{Z} comprises an oligonucleotide-based agent.

In some embodiments, the compound of Formula (II) is selected from the group consisting of LP 38b, LP 39b, LP 41b, LP 42b, LP 43b, LP 44b, LP 45b, LP 47b, LP 53b, LP 54b, LP 55b, LP 57b, LP 58b, LP 59b, LP 60b, LP 62b, LP 101b, LP 104b, LP 106b, LP 107b, LP 108b, LP 109b, and LP 111b as set forth in Table 16, or a pharmaceutically acceptable salt of any of these compounds, wherein each R_{Z} comprises an oligonucleotide-based agent.

Another aspect of the present invention provides a compound of Formula (III):

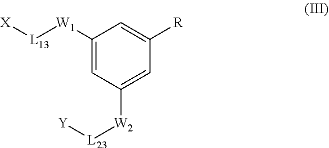

or a pharmaceutically acceptable salt thereof, wherein R, X, and Y are as defined for any embodiments of the compound of Formula (I), (Ia), (Ib), (Ib1), (Ic), or (II); L₁₃ is L₁ as defined for any embodiments of the compound of Formula (I), (Ia), (Ib), (Ib1), (Ic), or (Id), or L₁₃ is L₁₂ as defined for any embodiments of the compound of Formula (II); L₂₃ is L₂ as defined for any embodiments of the compound of Formula (I), (Ia), (Ib), (Ib1), (Ic), or (Id), or L₂₃ is L₂₂ as defined for any embodiments of the compound of Formula (II); W₁ is —C(O)NR₁— or —OCH₂CH₂NR₁C(O)—, wherein R₁ is hydrogen or C₁₋₆ alkyl; and W₂ is —C(O)NR₂— or —OCH₂CH₂NR₂C(O)—, wherein R₂ is hydrogen or C₁₋₆ alkyl.

In some embodiments, R is L_{A3}-R_{Z}; L_{A3} is a bond or a bivalent moiety connecting R_{Z} to the phenyl ring; R_{Z} comprises an oligonucleotide-based agent; W₁ is —C(O)NR₁— or —OCH₂CH₂NR₁C(O)—, wherein R₁ is hydrogen or C₁₋₆ alkyl; W₂ is —C(O)NR₂— or —OCH₂CH₂NR₂C(O)—, wherein R₂ is hydrogen or C₁₋₆ alkyl; L₁₃ and L₂₃ are each independently linkers comprising at least about 5 PEG units; and X and Y are each independently lipids comprising from about 10 to about 50 carbon atoms.

In some embodiments, each of L₁₃ and L₂₃ is independently selected from the group consisting of the moieties identified in Table 8.

TABLE 8
Example L₁₃ and L₂₃ moieties of the present invention.

| Name | Structure |
|---|---|
| Linker 1-3 | 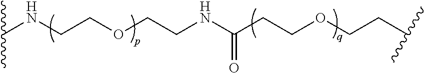 |
| Linker 2-3 | 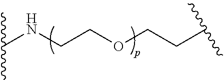 |
| Linker 3-3 | 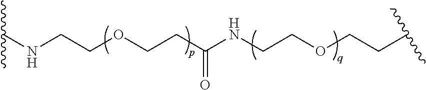 |

wherein, p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and each [symbol] indicates a point of connection to X, Y, W₁, or W₂; provided that:
(i) in Linker 1-3 and Linker 3-3, p+q≥5; and
(ii) in Linker 2-3, p≥5.

In some embodiments, each p is independently 20, 21, 22, 23, 24, or 25. In some embodiments, each p is independently 23 or 24. In some embodiments, each p is 23. In some embodiments, each p is 24. In some embodiments, each q is independently 20, 21, 22, 23, 24, or 25. In some embodiments, each q is 24.

In some embodiments, at least one of X and Y is selected from the group consisting of the moieties identified in Table 3, wherein each [symbol] indicates a point of connection to L₁₃ or L₂₃. In some embodiments, each of X and Y is independently selected from the group consisting of the moieties identified in Table 3, wherein each [symbol] indicates a point of connection to L₁₃ or L₂₃.

In some embodiments, at least one of X and Y is selected from the group consisting of the moieties identified in Table 9. In some embodiments, each of X and Y is independently selected from the group consisting of the moieties identified in Table 9.

TABLE 9
Example X and Y moieties of the compound of Formula (III).

| Name | Structure |
|---|---|
| Lipid 3 | 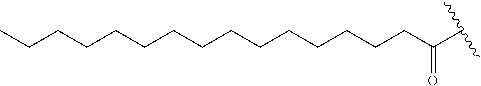 [SMILES: *C(=O)CCCCCCCCCCCCCCC] |
| Lipid 19 | 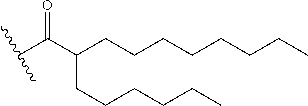 [SMILES: *C(=O)C(CCCCCC)CCCCCCCC] |

wherein [symbol] indicates a point of connection to L₁₃ or L₂₃.

In some embodiments, L_{A3} comprises at least one PEG unit. In some embodiments, L_{A3} is free of any PEG units. In some embodiments, L_{A3} comprises —C(O)—, —C(O)N(H)—, —N(H)C(O)—, optionally substituted alkoxy, or an optionally substituted alkyleneheterocyclyl. In some embodiments, L_{A3} is a bond.

In some embodiments, L_{A3} is selected from the group consisting of the moieties identified in Table 10.

TABLE 10
Example L_{A3} moieties of the present invention.

| Name | Structure |
|---|---|
| Tether 1-3 | 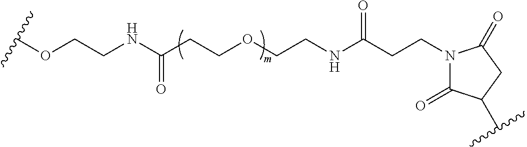 |
| Tether 2-3 | 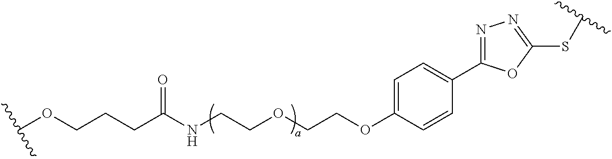 |
| Tether 3-3 | 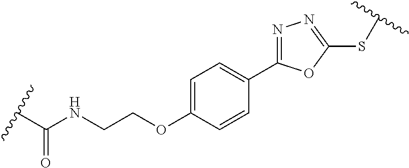 [SMILES: *Sc1nnc(-c2ccc(OCCNC(*)=O)cc2)o1] |
| Tether 4-3 | 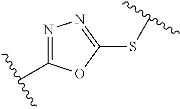 [SMILES: *Sc1nnc(*)o1] |
| Tether 5-3 | 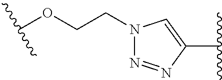 [SMILES: *OCCn1cc(*)nn1] |

wherein, each of m and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to R_{Z} or the phenyl ring of Formula (III).

In some embodiments, m is 1, 2, 3, 4, 5, 20, 21, 22, 23, or 25. In some embodiments, m is 1, 2, 3, 4, or 5. In some embodiments, m is 2 or 4. In some embodiments, a is 2, 3, 4, or 5. In some embodiments, a is 3.

In some embodiments, each of R¹ and R² is independently hydrogen or C₁₋₃ alkyl (e.g., methyl, ethyl, or n-propyl). In some embodiments, both of R¹ and R² is hydrogen.

In some embodiments, the compound of Formula (III) is selected from the group consisting of LP 110a, LP 124a, LP 130a, and LP 220a as set forth in Table 14, or a pharmaceutically acceptable salt of any of these compounds, wherein each R is L_{A3}-R_{Z}; L_{A3} is a bond or a bivalent moiety connecting R_{Z} to the phenyl ring; and R_{Z} comprises an oligonucleotide-based agent.

In some embodiments, the compound of Formula (III) is selected from the group consisting of LP 110b, LP 124b, LP 130b, LP 143b, LP 220b, LP 221b, and LP 240b as set forth in Table 16, or a pharmaceutically acceptable salt of any of these compounds, wherein each R_{Z} comprises an oligonucleotide-based agent.

Another aspect of the present invention provides a compound of Formula (IIIa):

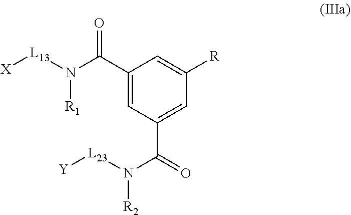

or a pharmaceutically acceptable salt thereof, wherein each of R, X, and Y is as defined for any embodiments of the compound of Formula (I), (Ia), (Ib), (Ib1), (Ic), (II), or (III); L₁₃ is L₁ as defined for any embodiments of the compound of Formula (I), (Ia), (Ib), (Ib1), (Ic), or (Id), L₁₃ is L₁₂ as defined for any embodiments of the compound of Formula (II), or L₁₃ is as defined in any embodiments of the compound of Formula (III); L₂₃ is L₂ as defined for any embodiments of the compound of Formula (I), (Ia), (Ib), (Ib1), (Ic), or (Id), L₂₃ is L₂₂ as defined for any embodiments of the compound of Formula (II), or L₁₃ is as defined in any embodiments of the compound of Formula (III); and each of R₁ and R₂ are as defined in any embodiments of the compound of Formula (II) or (III).

In some embodiments, R is L_{A3}-R_{Z}; L_{A3} is a bond or a bivalent moiety connecting R_{Z} to the phenyl ring; R_{Z} comprises an oligonucleotide-based agent; R₁ and R₂ are each independently hydrogen or C₁₋₆ alkyl (e.g., methyl, ethyl, n-propyl, n-butyl, or n-pentyl); L₁₃ and L₂₃ are each independently linkers comprising at least about 5 PEG units; and X and Y are each independently lipids comprising from about 10 to about 50 carbon atoms.

In some embodiments, each of L₁₃ and L₂₃ is selected from the group consisting of Linker 1-3 and Linker 2-3 as set forth in Table 8, wherein each [symbol] indicates a point of connection to X, Y, —NR₁—, or —NR₂— in Formula (IIIa), provided that:
(i) in Linker 1-3, p+q≥5; and
(ii) in Linker 2-3, p≥5.

In some embodiments, one of L₁₃ and L₂₃ is Linker 1-3 and the other is Linker 2-3. In some embodiments, each of L₁₃ and L₂₃ is Linker 1-3. In some embodiments, each of L₁₃ and L₂₃ is Linker 2-3.

In some embodiments, each p is independently 23 or 24. In some embodiments, each p is 23. In some embodiments, each p is 24. In some embodiments, q is 24.

In some embodiments, at least one of X and Y is selected from the group consisting of Lipid 3 and Lipid 19 as set forth in Table 9, wherein each [symbol] indicates a point of connection to L₁₃ or L₂₃ in Formula (IIIa). In some embodiments, each of X and Y is independently selected from the group consisting of Lipid 3 and Lipid 19. In some embodiments, one of X and Y is Lipid 3 and the other is Lipid 19. In some embodiments, each of X and Y is Lipid 3. In some embodiments, each of X and Y is Lipid 19.

In some embodiments, L_{A3} is selected from the group consisting of Tether 1-3, Tether 2-3, and Tether 5-3 as set forth in Table 10, wherein each [symbol] indicates a point of connection to R_{Z} or the phenyl ring of Formula (IIIa). In some embodiments, L_{A3} is Tether 1-3. In some embodiments, L_{A3} is Tether 2-3. In some embodiments, L_{A3} is Tether 5-3.

In some embodiments, m is 1, 2, 3, 4, 5, 20, 21, 22, 23, or 25. In some embodiments, m is 1, 2, 3, 4, or 5. In some embodiments, m is 2 or 4. In some embodiments, a is 2, 3, 4, or 5. In some embodiments, a is 3.

In some embodiments, each of R¹ and R² is independently hydrogen or C₁₋₃ alkyl. In some embodiments, each of R¹ and R² is hydrogen.

In some embodiments, the compound of Formula (IIIa) is selected from the group consisting of LP 110a, LP 124a, and LP 130a as set forth in Table 14, or a pharmaceutically acceptable salt of any of these compounds, wherein each R is L_{A3}-R_{Z}; L_{A3} is a bond or a bivalent moiety connecting R_{Z} to the phenyl ring; and R_{Z} comprises an oligonucleotide-based agent.

In some embodiments, the compound of Formula (IIIa) is selected from the group consisting of LP 110b, LP 124b, LP 130b, LP 143b, and LP 240b as set forth in Table 16, or a pharmaceutically acceptable salt of any of these compounds, wherein each R_{Z} comprises an oligonucleotide-based agent.

Another aspect of the present invention provides a compound of Formula (IIIb):

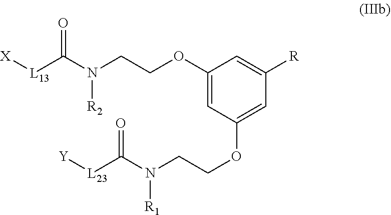

or a pharmaceutically acceptable salt thereof, wherein R, X, and Y are as defined for any embodiments of the compound of Formula (I), (Ia), (Ib), (Ib1), (Ic), (II), (III), or (IIIa); L₁₃ is L₁ as defined for any embodiments of the compound of Formula (I), (Ia), (Ib), (Ib1), (Ic), or (Id), L₁₃ is L₁₂ as defined for any embodiments of the compound of Formula (II), or L₁₃ is as defined in any embodiments of the compound of Formula (III) or (IIIa); L₂₃ is L₂ as defined for any embodiments of the compound of Formula (I), (Ia), (Ib), (Ib1), (Ic), or (Id), L₂₃ is L₂₂ as defined for any embodiments of the compound of Formula (II), or L₂₃ is as defined in any embodiments of the compound of Formula (III) or (IIIa); and each of R₁ and R₂ are as defined in any embodiments of the compound of Formula (II), (III), or (IIIa).

In some embodiments, R is L_{A3}-R_{Z}; L_{A3} is a bond or a bivalent moiety connecting R_{Z} to the phenyl ring; R_{Z} comprises an oligonucleotide-based agent; R₁ and R₂ are each independently selected from hydrogen or C₁₋₆ alkyl; L₁₃ and L₂₃ are each independently linkers comprising at least about 5 PEG units; and X and Y are each independently lipids comprising from about 10 to about 50 carbon atoms.

In some embodiments, each of L₁₃ and L₂₃ is Linker 3-3 as set forth in Table 8, wherein each [symbol] indicates a point of connection to X, Y, or —C(O)—, provided that in Linker 3-3, p+q≥5.

In some embodiments, p is 23 or 24. In some embodiments, p is 23. In some embodiments, p is 24. In some embodiments, q is 24.

In some embodiments, each of X and Y is Lipid 3 as set forth in Table 9, wherein each [symbol] indicates a point of connection to L₁₃ or L₂₃.

In some embodiments, L_{A3} is selected from the group consisting of Tether 3-3 and Tether 4-3 as set forth in Table 10, wherein each [symbol] indicates a point of connection to R_{Z} or the phenyl ring of Formula (IIIb). In some embodiments, L_{A3} is Tether 3-3. In some embodiments, L_{A3} is Tether 4-3.

In some embodiments, each of R¹ and R² is independently hydrogen or C₁₋₃ alkyl. In some embodiments, each of R¹ and R² is hydrogen.

In some embodiments, the compound of Formula (IIIb) is LP 220a as set forth in Table 14, or a pharmaceutically acceptable salt thereof, wherein R is L_{A3}-R_{Z}; L_{A3} is a bond or a bivalent moiety connecting R_{Z} to the phenyl ring; and R_{Z} comprises an oligonucleotide-based agent.

In some embodiments, the compound of Formula (IIIb) is selected from the group consisting of LP 220b and LP 221b as set forth in Table 16, or a pharmaceutically acceptable salt of any of these compounds, wherein each R_{Z} comprises an oligonucleotide-based agent.

Another aspect of the invention provides a compound of Formula (IV):

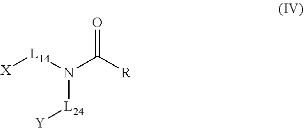

or a pharmaceutically acceptable salt thereof, wherein R, X, and Y are as defined for any embodiments of the compound of Formula (I), (Ia), (Ib), (Ib1), (Ic), (II), (III), (IIIa), or (IIIb); L₁₄ is L₁ as defined for any embodiments of the compound of Formula (I), (Ia), (Ib), (Ib1), or (Ic), L₁₄ is L₁₂ as defined for any embodiments of the compound of Formula (II), or L₁₄ is L₁₃ as defined in any embodiments of the compound of Formula (III), (IIIa), or (IIIb); L₂₄ is L₂ as defined for any embodiments of the compound of Formula (I), (Ia), (Ib), (Ib1), or (Ic), L₂₄ is L₂₂ as defined for any embodiments of the compound of Formula (II), or L₂₄ is L₂₃ as defined in any embodiments of the compound of Formula (III), (IIIa), or (IIIb).

In some embodiments, R is L_{A4}-R_{Z}; L_{A4} is a bond or a bivalent moiety connecting R_{Z} to —C(O)—; R_{Z} comprises an oligonucleotide-based agent; L₁₄ and L₂₄ are each independently linkers comprising at least about 5 PEG units; and X and Y are each independently lipids comprising from about 10 to about 50 carbon atoms.

In some embodiments, each of L₁₄ and L₂₄ is independently selected from the group consisting of the moieties identified in Table 11.

TABLE 11
Example L₁₄ and L₂₄ moieties of the present invention.

| Name | Structure |
|---|---|
| Linker 1-4 | 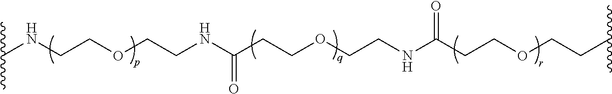 |
| Linker 2-4 | 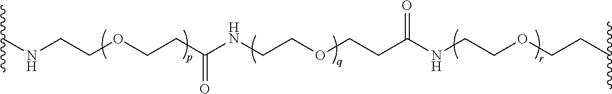 |
| Linker 3-4 | 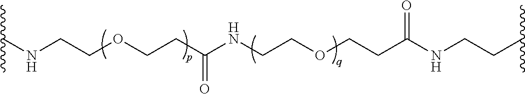 |
| Linker 4-4 | 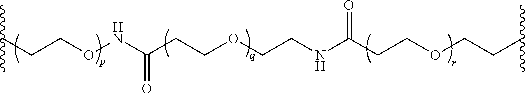 |
| Linker 5-4 | 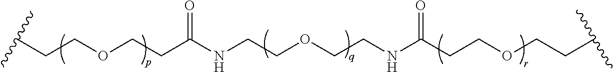 |

wherein each p is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; each q is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; each r is independently 1, 2, 3, 4, 5, 6, 7, 8, 9, or 10; and each [symbol] indicates a point of connection to X, Y, or

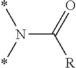

of Formula (IV), wherein each * indicates the point of attachment to L₁₄ or L₂₄; provided that:
(i) in Linker 1-4, Linker 2-4, and Linker 4-4, p+q+r≥5; and
(ii) in Linker 3-4, p+q≥5.

In some embodiments, each p is independently 20, 21, 22, 23, 24, or 25. In some embodiments, each p is independently 23 or 24. In some embodiments, each p is 23. In some embodiments, each p is 24. In some embodiments, each q is independently 20, 21, 22, 23, 24, or 25. In some embodiments, each q is independently 23 or 24. In some embodiments, each q is 24. In some embodiments, each q is 23. In some embodiments, each r is independently 2, 3, 4, 5, or 6. In some embodiments, each r is 4.

In some embodiments, at least one of X and Y is selected from the group consisting of the moieties identified in Table 3, wherein each [symbol] indicates a point of connection to L₁₄ or L₂₄. In some embodiments, each of X and Y is independently selected from the group consisting of the moieties identified in Table 3, wherein each [symbol] indicates a point of connection to L₁₄ or L₂₄.

In some embodiments, at least one of X and Y is selected from the group consisting of the moieties identified in Table 12. In some embodiments, each of X and Y is independently selected from the group consisting of the moieties identified in Table 12.

TABLE 12
Example X and Y moieties of the compound of Formula (IV).

| Name | Structure |
|---|---|
| Lipid 1 | 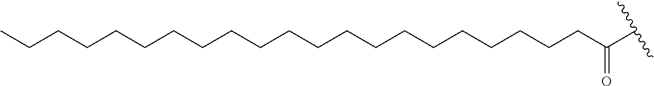 [SMILES: *C(=O)CCCCCCCCCCCCCCCCCCCCC] |
| Lipid 2 | 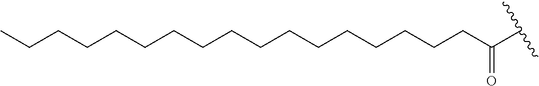 [SMILES: *C(=O)CCCCCCCCCCCCCCCCC] |
| Lipid 3 | 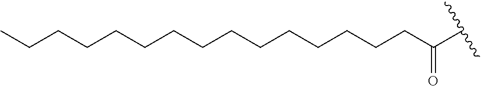 [SMILES: *C(=O)CCCCCCCCCCCCCCC] |
| Lipid 5 | 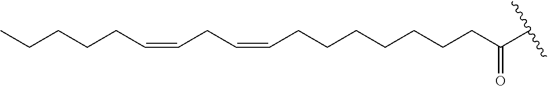 [SMILES: *C(=O)CCCCCCC/C=C\C/C=C\CCCCC] |
| Lipid 8 | 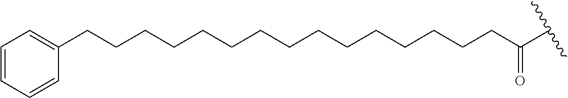 [SMILES: *C(=O)CCCCCCCCCCCCCCCc1ccccc1] |
| Lipid 9 | 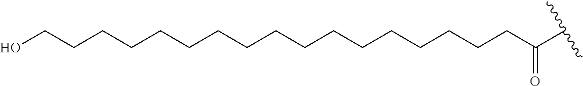 [SMILES: *C(=O)CCCCCCCCCCCCCCCCCO] |
| Lipid 10 | 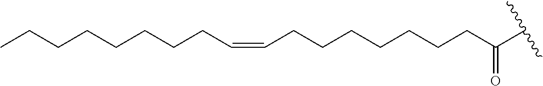 [SMILES: *C(=O)CCCCCCC/C=C\CCCCCCCC] |
| Lipid 11 | 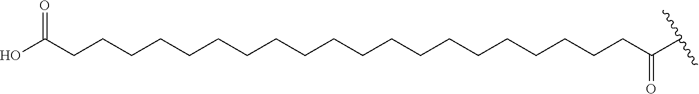 [SMILES: *C(=O)CCCCCCCCCCCCCCCCCCCCC(=O)O] |
| Lipid 12 | 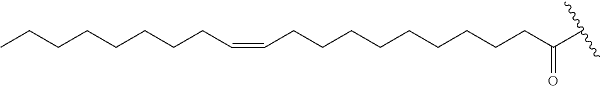 [SMILES: *C(=O)CCCCCCCCC/C=C\CCCCCCCC] |
| Lipid 15 | 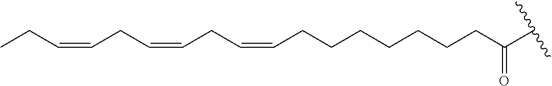 [SMILES: *C(=O)CCCCCCC/C=C\C/C=C\C/C=C\CC] |
| Lipid 16 | 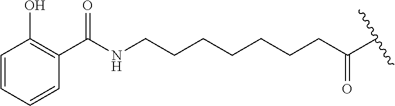 [SMILES: *C(=O)CCCCCCCNC(=O)c1ccccc1O] |
| Lipid 17 | 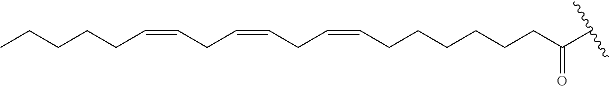 [SMILES: *C(=O)CCCCCC/C=C\C/C=C\C/C=C\CCCCC] |
| Lipid 18 | 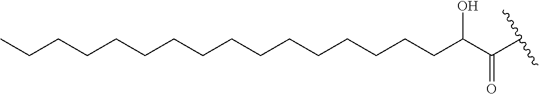 [SMILES: *C(=O)C(O)CCCCCCCCCCCCCCCC] |
| Lipid 19 | 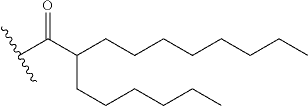 [SMILES: *C(=O)C(CCCCCC)CCCCCCCC] |
| Lipid 20 | 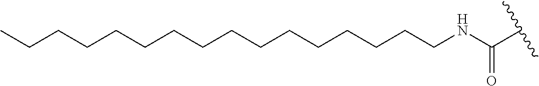 [SMILES: *C(=O)NCCCCCCCCCCCCCCCC] |
| Lipid 21 | 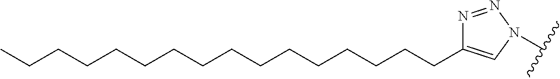 [SMILES: *n1cc(CCCCCCCCCCCCCCCC)nn1] |
| Lipid 22 | 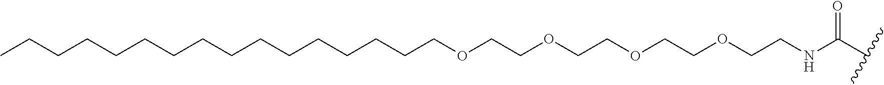 [SMILES: *C(=O)NCCOCCOCCOCCOCCCCCCCCCCCCCCCC] |
| Lipid 23 | 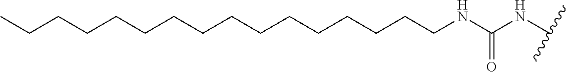 [SMILES: *NC(=O)NCCCCCCCCCCCCCCCC] |
| Lipid 24 | 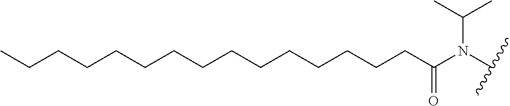 [SMILES: *N(C(=O)CCCCCCCCCCCCCCC)C(C)C] |

wherein [symbol] indicates a point of connection to L₁₄ or L₂₄.

In some embodiments, L_{A4} comprises at least one PEG unit. In some embodiments, L_{A4} is free of any PEG units. In some embodiments, L_{A4} comprises —C(O)—, —C(O)NH—, optionally substituted alkoxy, or an optionally substituted alkyleneheterocyclyl. In some embodiments, L_{A4} is a bond.

In some embodiments, L_{A4} is selected from the group consisting of the moieties identified in Table 13.

TABLE 13
Example L_{A4} moieties of the present invention.

| Name | Structure |
|---|---|
| Tether 1-4 | 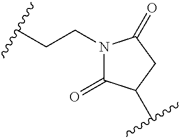 [SMILES: *CCN1C(=O)CC(*)C1=O] |
| Tether 2-4 | 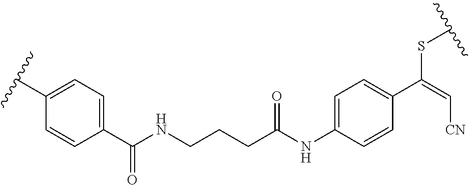 [SMILES: *S/C(=C/C#N)c1ccc(NC(=O)CCCNC(=O)c2ccc(*)cc2)cc1] |
| Tether 3-4 | 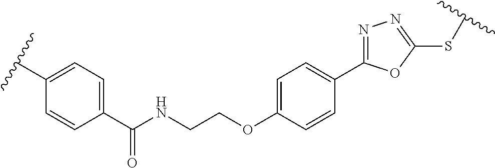 [SMILES: *Sc1nnc(-c2ccc(OCCNC(=O)c3ccc(*)cc3)cc2)o1] |
| Tether 4-4 | 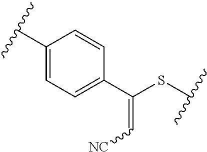 [SMILES: *SC(=CC#N)c1ccc(*)cc1] |
| Tether 5-4 | 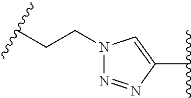 [SMILES: *CCn1cc(*)nn1] |
| Tether 6-4 | 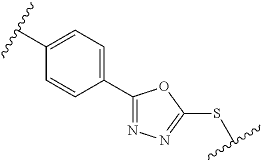 [SMILES: *Sc1nnc(-c2ccc(*)cc2)o1] |

wherein each [symbol] indicates a point of connection to R_{Z} or the —C(O)— of Formula (IV).

In some embodiments, the compound of Formula (IV) is selected from the group consisting of LP 1a, LP 28a, LP 29a, LP 48a, LP 49a, LP 56a, LP 61a, LP 87a, LP 89a, LP 90a, LP 92a, LP 93a, LP 94a, LP 95a, LP 102a, LP 103a, LP 223a, LP 225a, LP 246a, LP 339a, LP 340a, LP 357a, and LP 358a as set forth in Table 14, or a pharmaceutically acceptable salt of any of these compounds, wherein each R is L_{A4}-R_{Z}; L_{A4} is a bond or a bivalent moiety connecting R_{Z} to —C(O)—; and R_{Z} comprises an oligonucleotide-based agent.

In some embodiments, the compound of Formula (IV) is selected from the group consisting of LP 1b, LP 28b, LP 29b, LP 48b, LP 49b, LP 56b, LP 61b, LP 87b, LP 89b, LP 90b, LP 92b, LP 93b, LP 94b, LP 95b, LP 102b, LP 103b, LP 223b, LP 224b, LP 225b, LP 226b, LP 238b, LP 246b, LP 247b, LP 339b, LP 340b, LP 357b, and LP 358b as set forth in Table 16, or a pharmaceutically acceptable salt of any of these compounds, wherein each R_{Z} comprises an oligonucleotide-based agent.

Another aspect of the invention provides a compound of Formula (IVa):

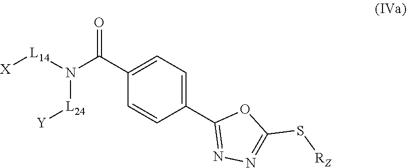

or a pharmaceutically acceptable salt thereof, wherein X and Y are as defined for any embodiments of the compound of Formula (I), (Ia), (Ib), (Ib1), (Ic), (II), (III), (IIIa), (IIIb), or (IV); L₁₄ and L₂₄ are as defined in any of the embodiments of the compound of Formula (IV); and R_{Z} comprises an oligonucleotide-based agent.

In some embodiments, R_{Z} comprises an oligonucleotide-based agent; each of L₁₄ and L₂₄ is independently selected from the group consisting of

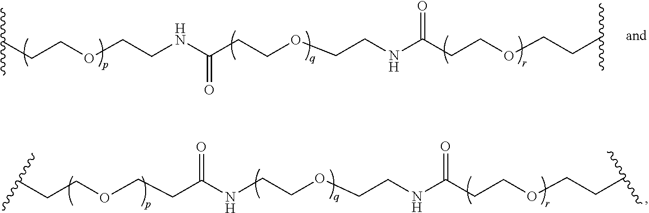

wherein each [symbol] indicates a point of connection to X, Y, or

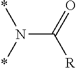

of Formula (IVa), each * indicates the point of attachment to L₁₄ or L₂₄, each p is independently 20, 21, 22, 23, 24, or 25, each q is independently 20, 21, 22, 23, 24, or 25, and each r is independently 2, 3, 4, 5, or 6; and each of X and Y is independently selected from the group consisting of

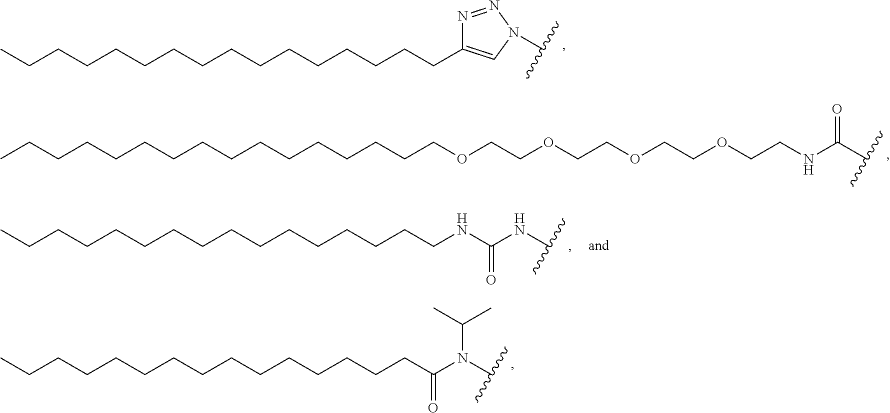

wherein [symbol] indicates a point of connection to L₁₄ or L₂₄.

In some embodiments, each p is independently 23 or 24. In some embodiments, each p is 23. In some embodiments, each p is 24. In some embodiments, each q is independently 23 or 24. In some embodiments, each q is 24. In some embodiments, each q is 23. In some embodiments, each r is 4.

In some embodiments, the compound of Formula (IVa) is selected from the group consisting of LP 339b, LP 340b, LP 357b, and LP 358b as set forth in Table 16, or a pharmaceutically acceptable salt of any of these compounds, wherein each R_{Z} comprises an oligonucleotide-based agent.

In another aspect of the invention, a compound is selected from the group consisting of the compounds identified in Table 14, or a pharmaceutically acceptable salt thereof.

TABLE 14
Example compounds of the present invention (compound number appears
before structure).
LP 1a

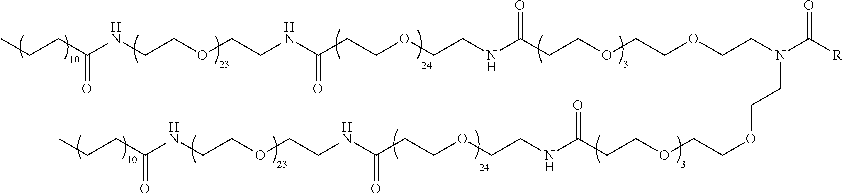

LP 28a

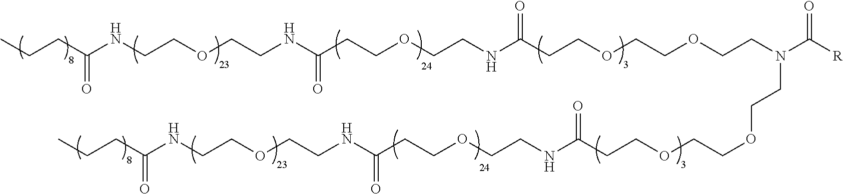

LP 29a

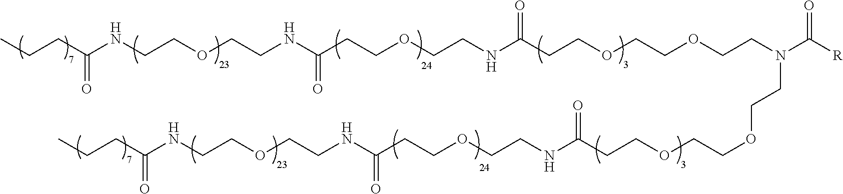

LP 38a

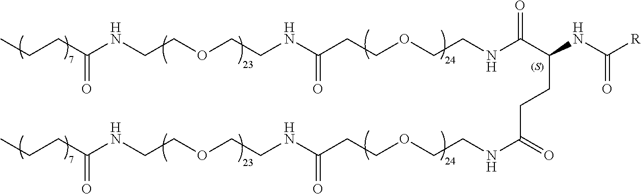

LP 39a

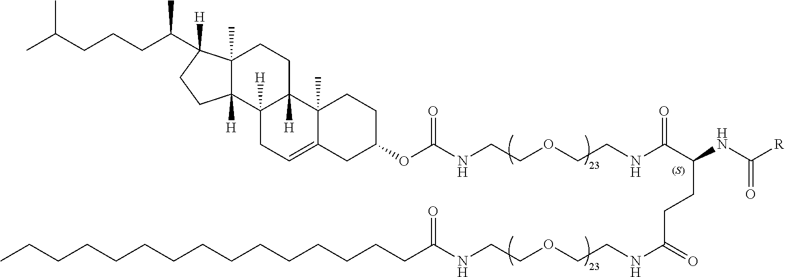

LP 43a

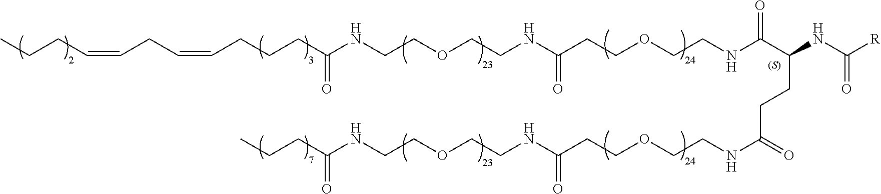

LP 44a

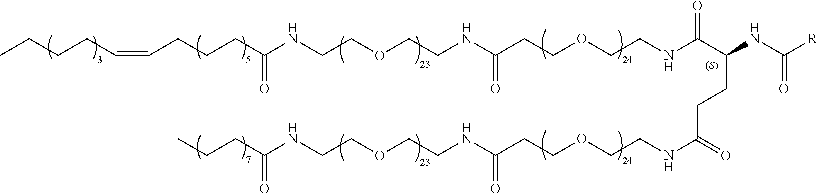

LP 45a

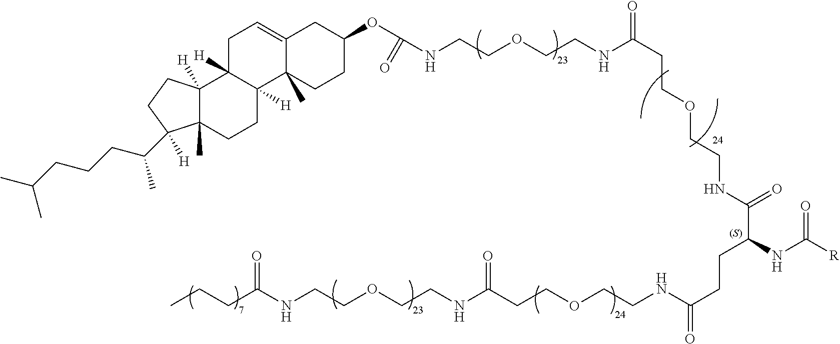

LP 47a

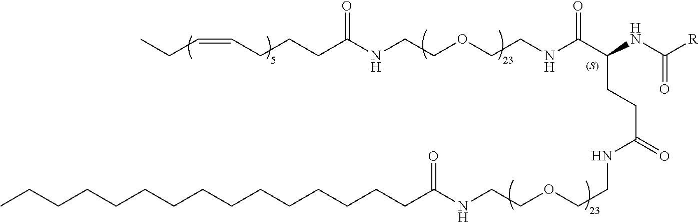

LP 48a

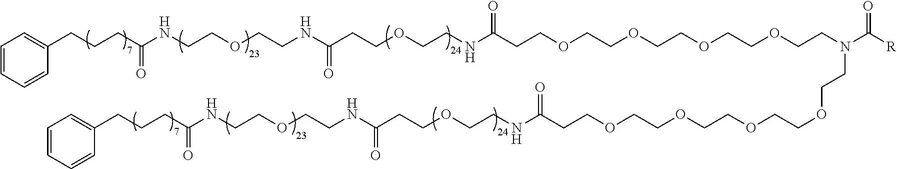

LP 49a

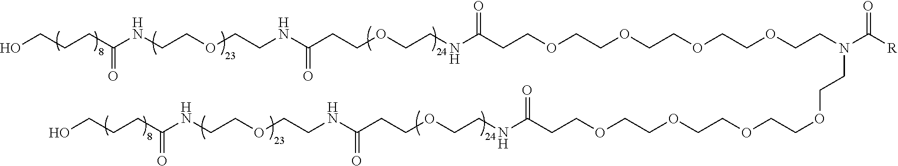

LP 53a

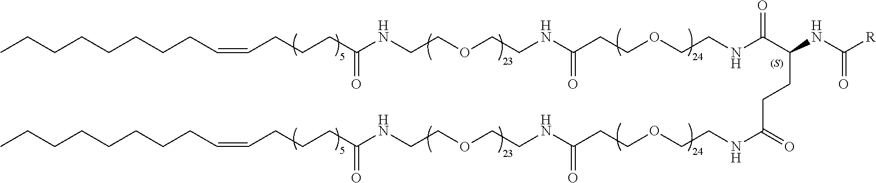

LP 54a

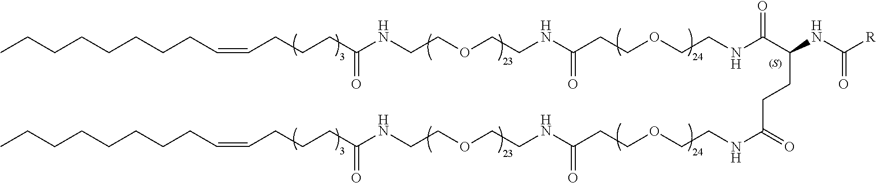

LP 55a

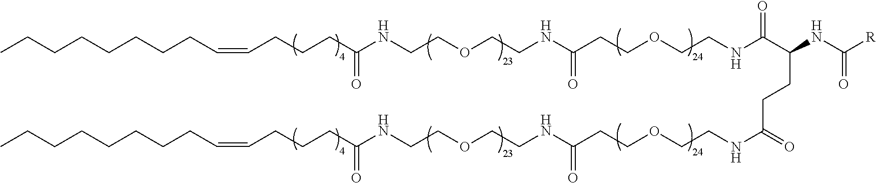

LP 56a

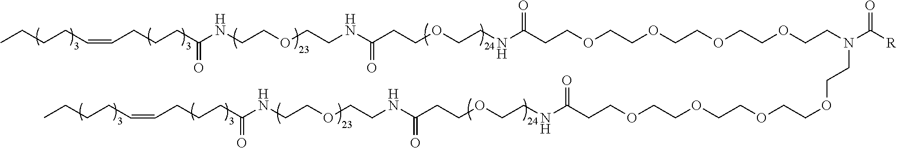

LP 57a

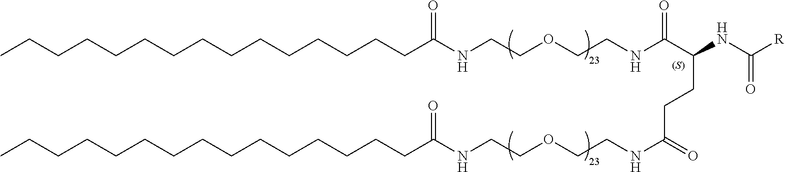

LP 58a

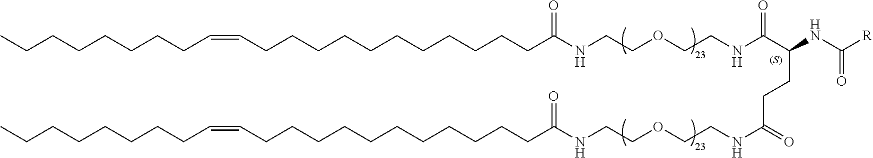

LP 59a

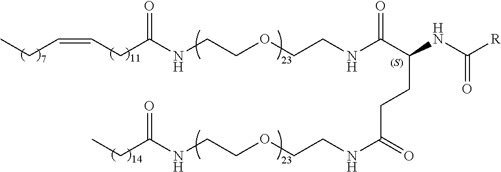

LP 61a

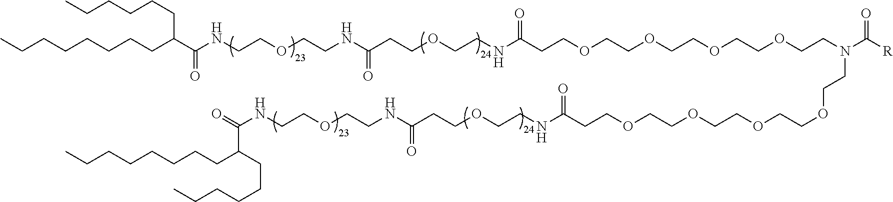

LP 62a

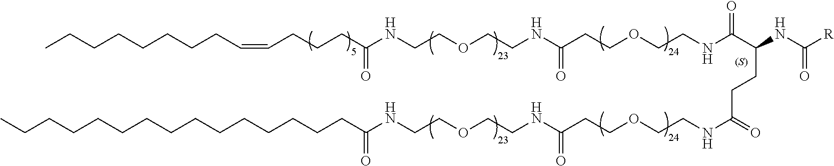

LP 87a

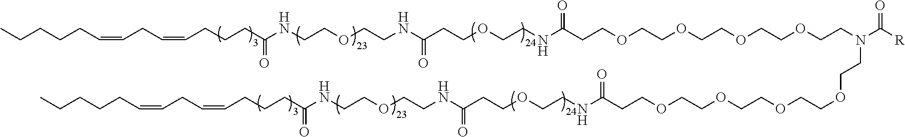

LP 89a

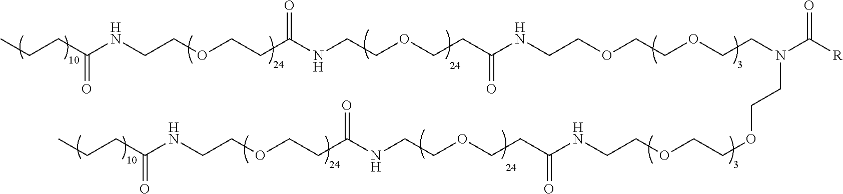

LP 90a

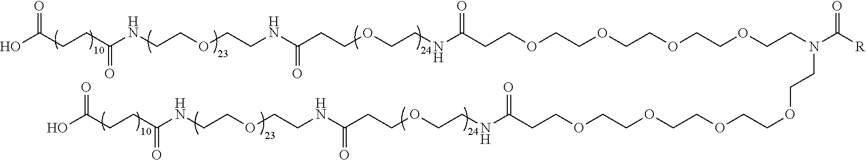

LP 92a

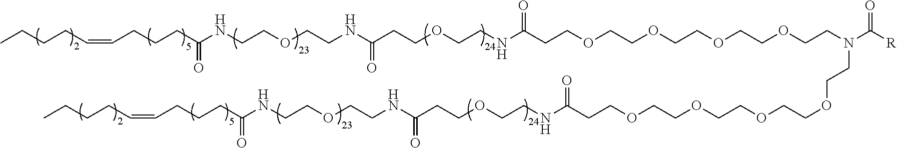

LP 93a

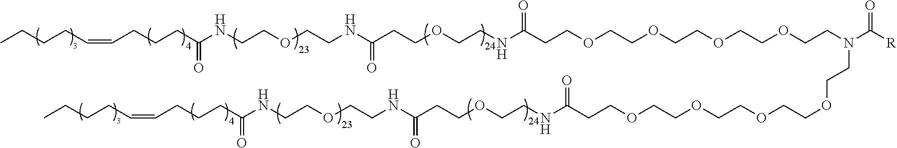

LP 94a

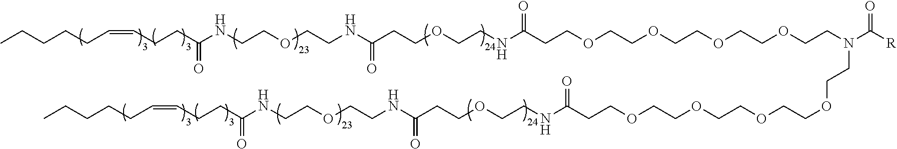

LP 95a

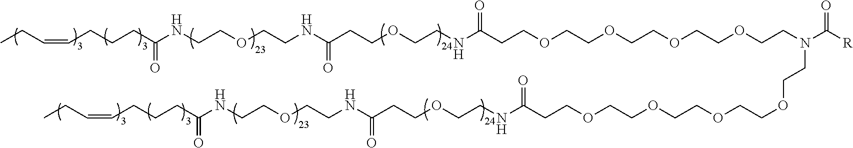

LP 101a

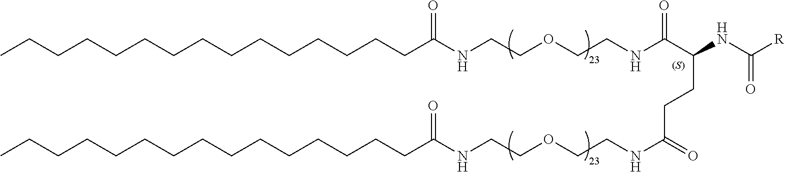

LP 102a

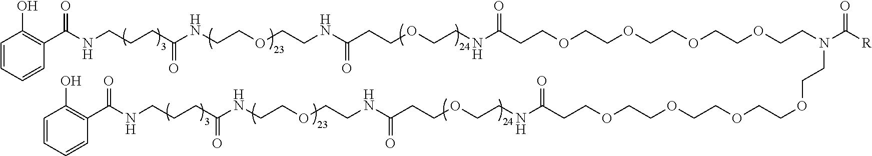

LP 103a

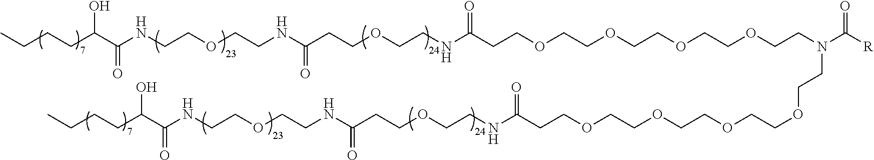

LP 104a

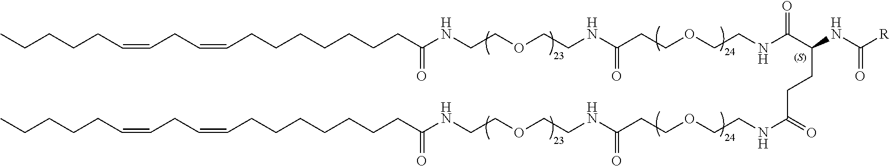

LP 110a

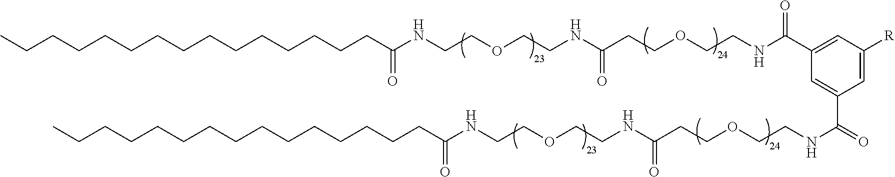

LP 111a

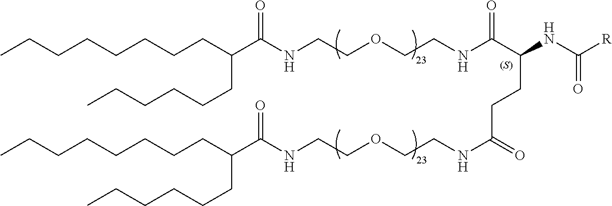

LP 124a

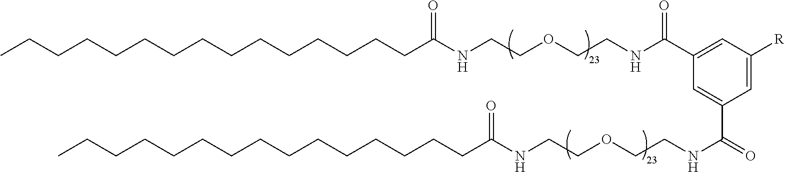

LP 130a

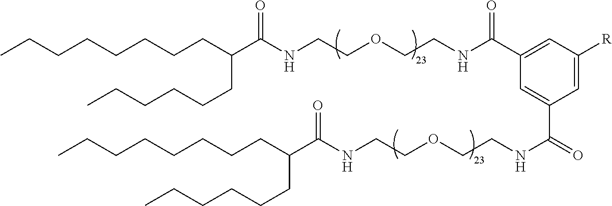

LP 210a

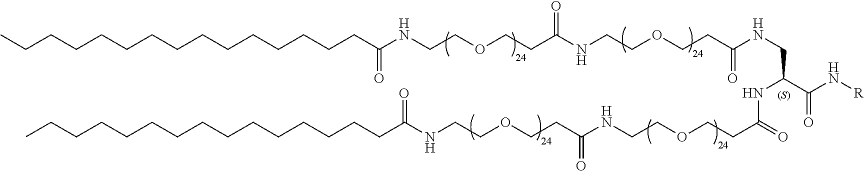

LP 217a

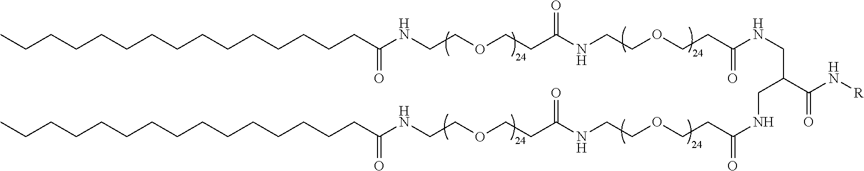

LP 220a

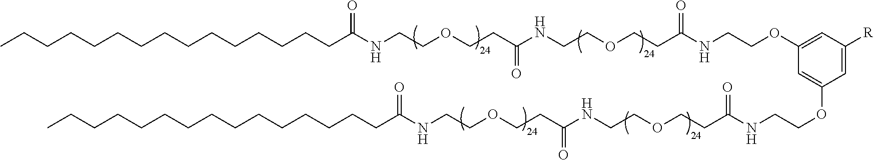

LP 223a

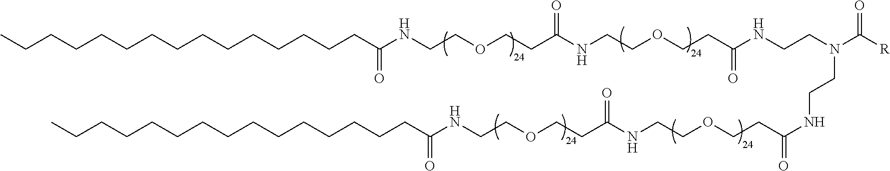

LP 225a

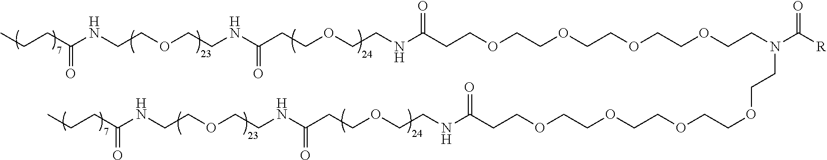

LP 246a

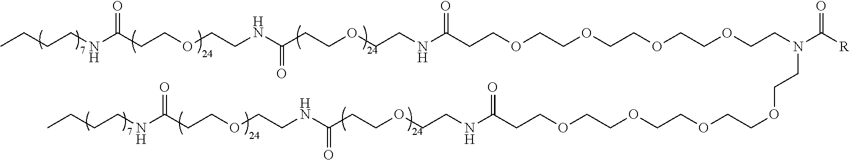

LP 339a

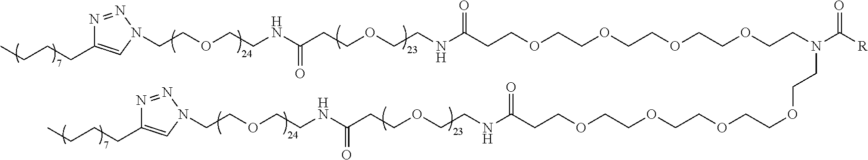

LP 340a

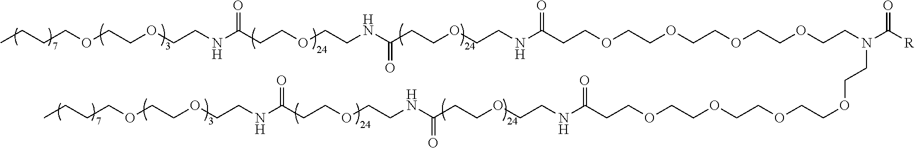

LP 357a

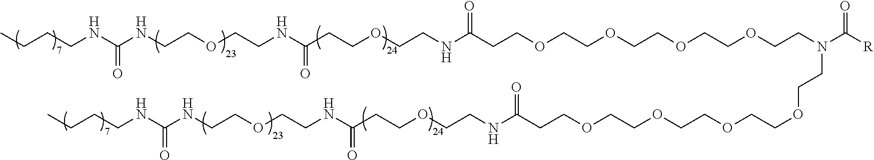

LP 358a

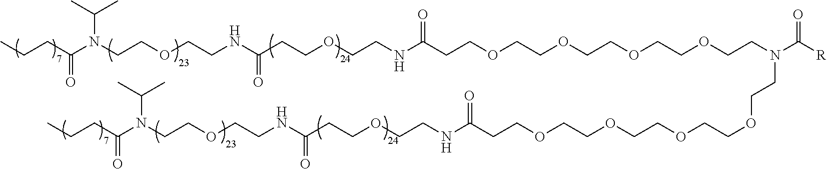

or a pharmaceutically acceptable salt of any of these compounds, wherein each R is as defined in any of the embodiments of the compound of Formula (I), (Ia), (Ib), (Ib1), (Ic), (Id), (II), (III), (IIIa), (IIIb), (IV), or (IVa).

In some embodiments, each R is L_{A}-R_{Z}; L_{A} is a bond or bivalent moiety for connecting R_{Z} to the rest of the compound; and R_{Z} comprises an oligonucleotide based agent.

In another aspect of the invention, a compound is selected from the group consisting of the compounds identified in Table 15, or a pharmaceutically acceptable salt thereof.

TABLE 15
Example compounds of the present invention (compound number appears
before structure).
LP 5a

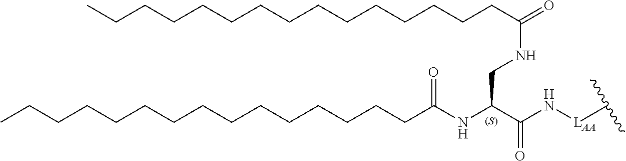

LP 33a

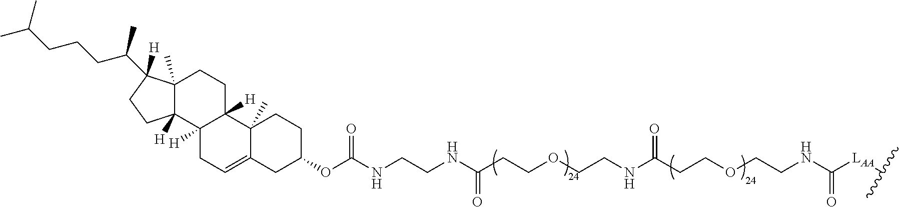

or a pharmaceutically acceptable salt of any of these compounds, wherein R is as defined in any of the embodiments of the compound of Formula (I), (Ia), (Ib), (Ib1), (Ic), (Id), (II), (III), (IIIa), (IIIb), (IV), or (IVa).

In some embodiments, each R is L_{A}-R_{Z}; L_{A} is a bond or bivalent moiety for connecting R_{Z} to the rest of the compound; and R_{Z} comprises an oligonucleotide based agent.

In another aspect of the invention, a compound is selected from the group consisting of the compounds identified in Table 16, or a pharmaceutically acceptable salt thereof.

TABLE 16
Example compounds of the present invention (compound number appears
before structure).
LP 1b

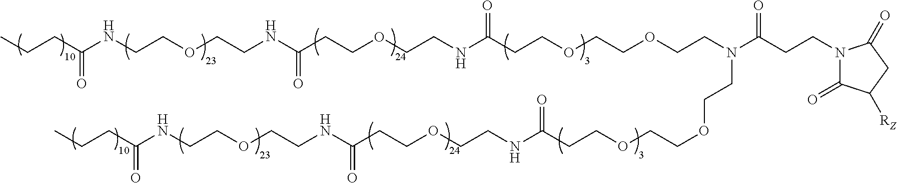

LP 28b

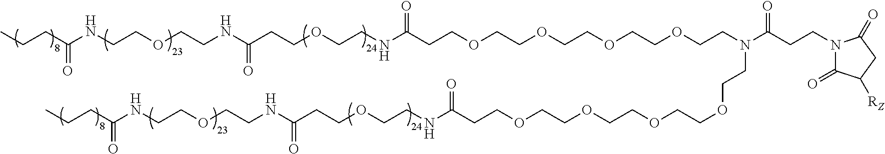

LP 29b

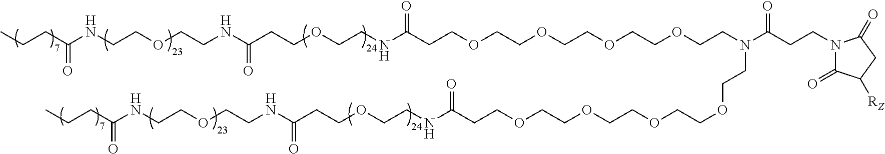

LP 38b

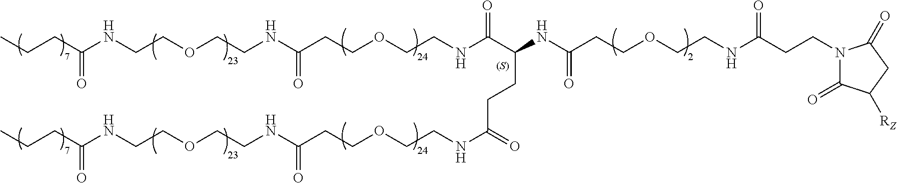

LP 39b

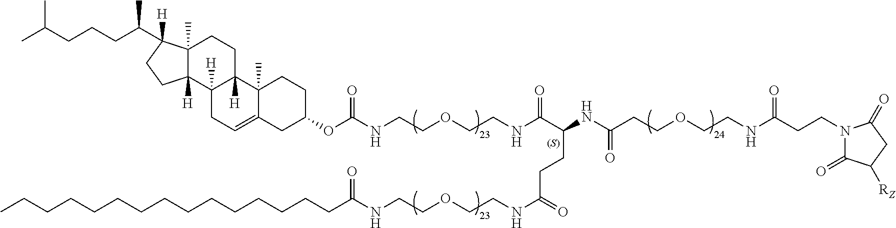

LP 41b

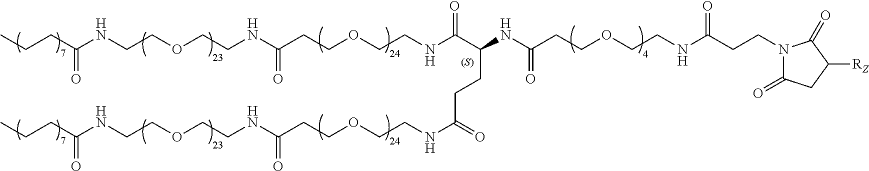

LP 42b

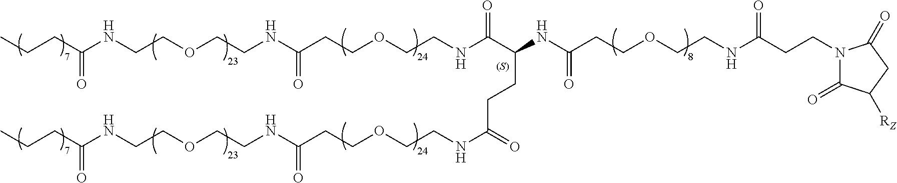

LP 43b

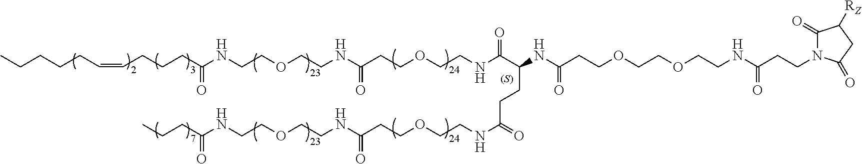

LP 44b

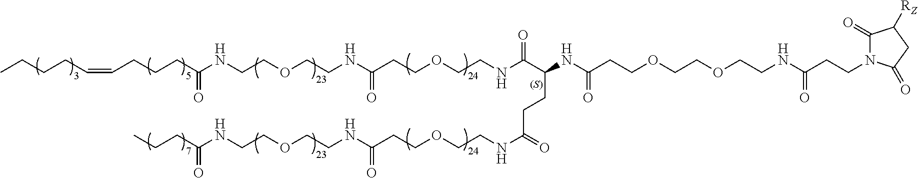

LP 45b

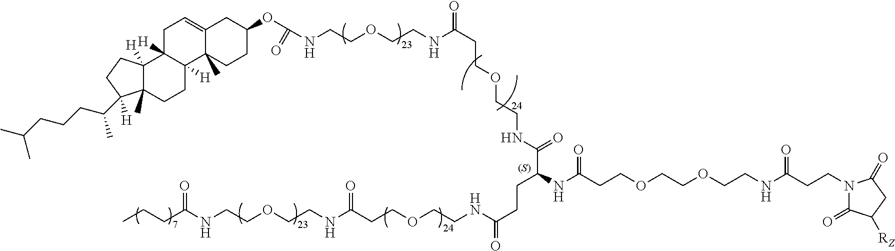

LP 47b

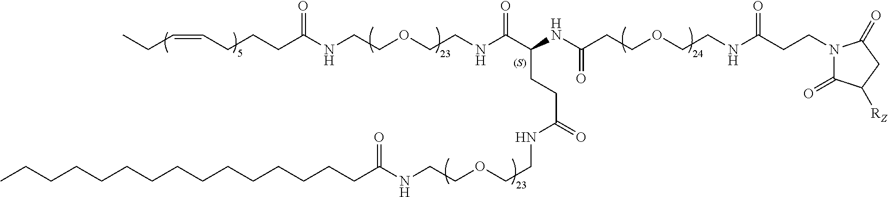

LP 48b

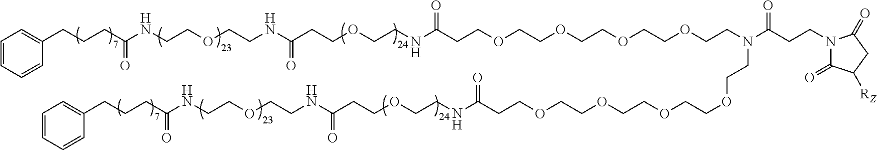

LP 49b

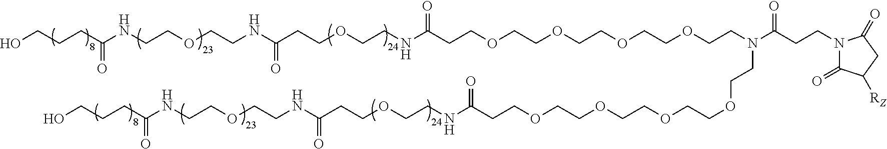

LP 53b

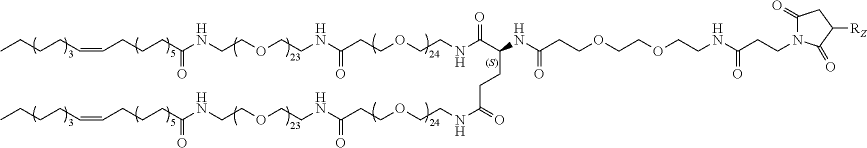

LP 54b

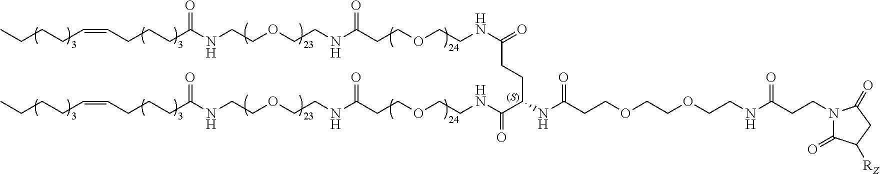

LP 55b

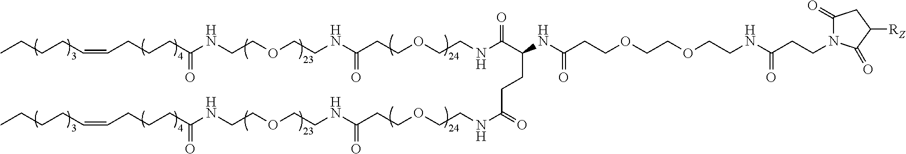

LP 56b

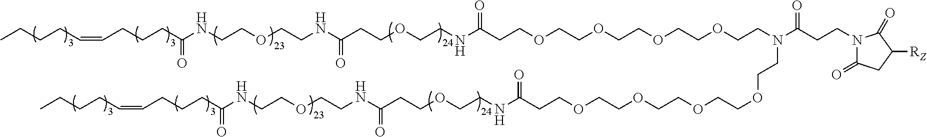

LP 57b

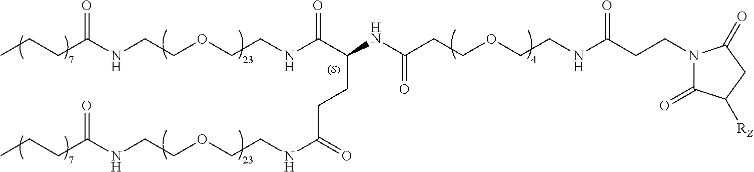

LP 58b

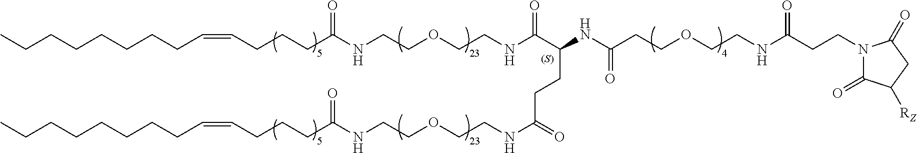

LP 59b

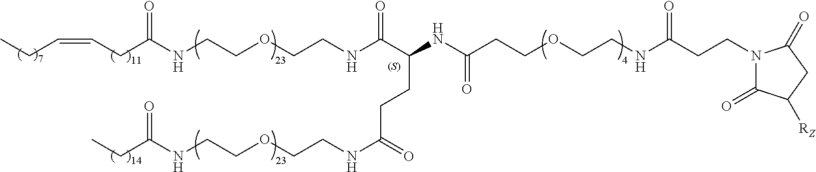

LP 60b

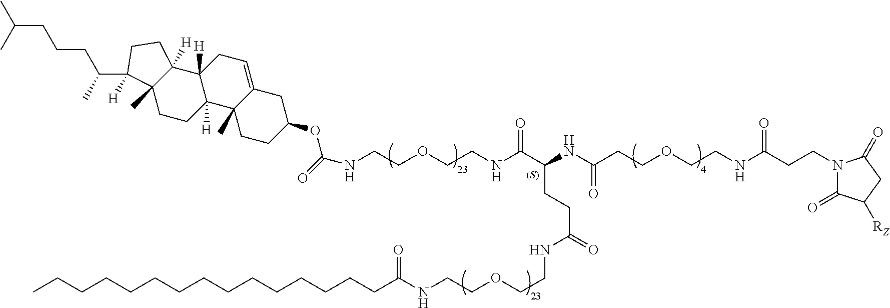

LP 61b

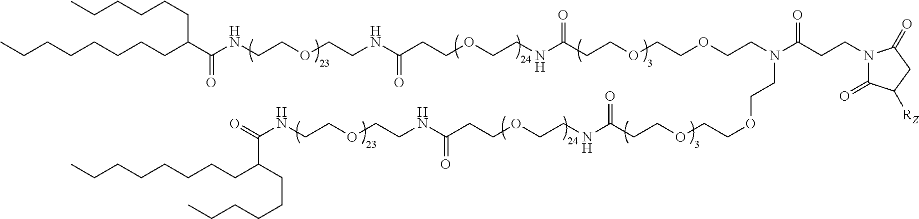

LP 62b

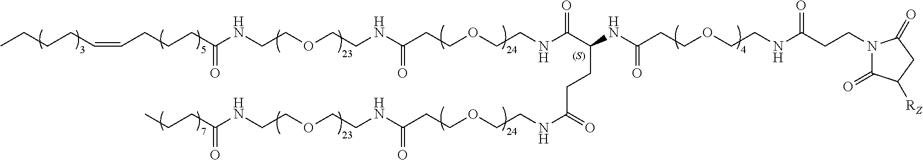

LP 87b

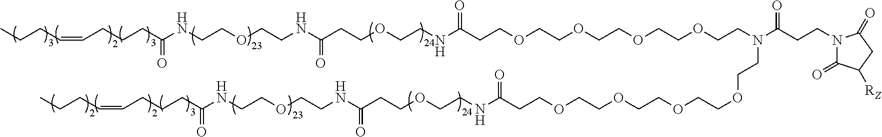

LP 89b

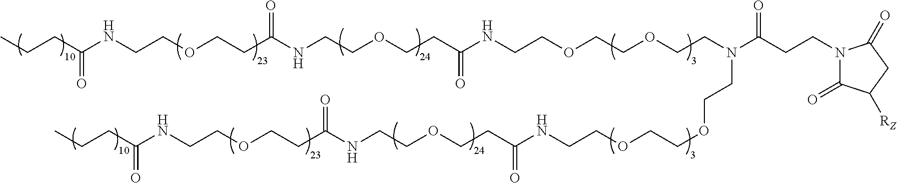

LP 90b

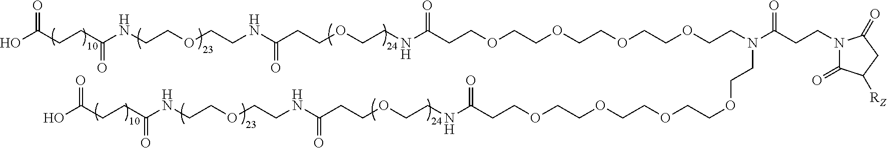

LP 92b

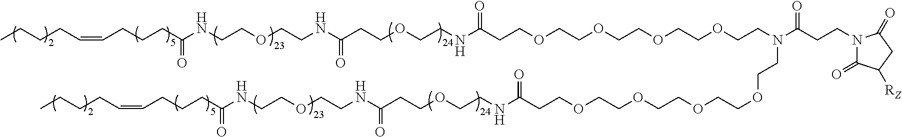

LP 93b

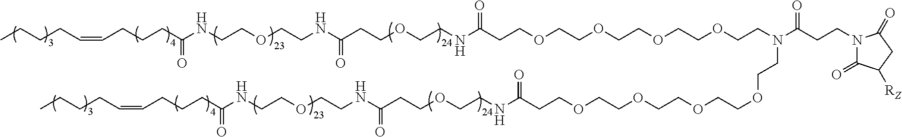

LP 94b

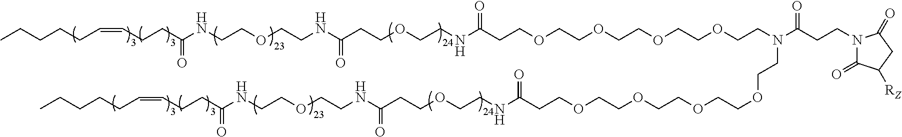

LP 95b

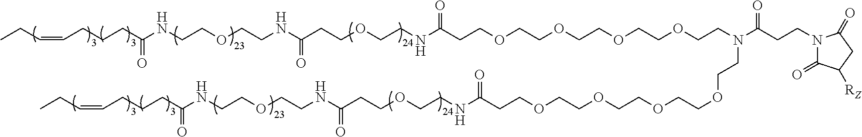

LP 101b

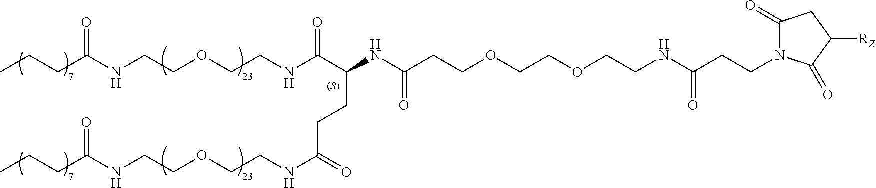

LP 102b

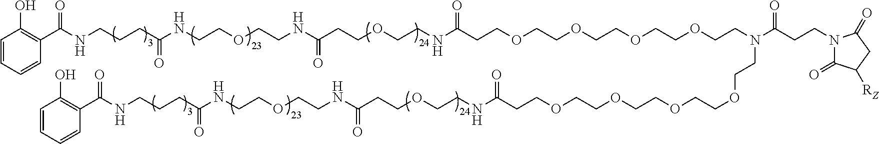

LP 103b

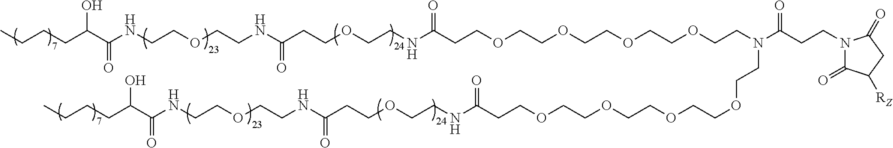

LP 104b

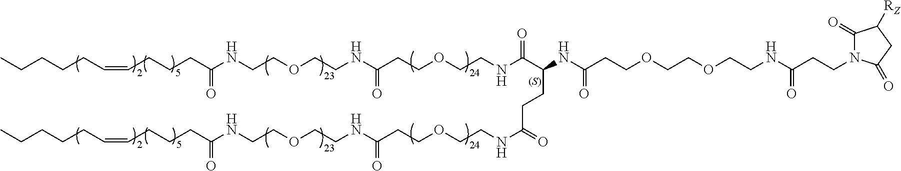

LP 106b

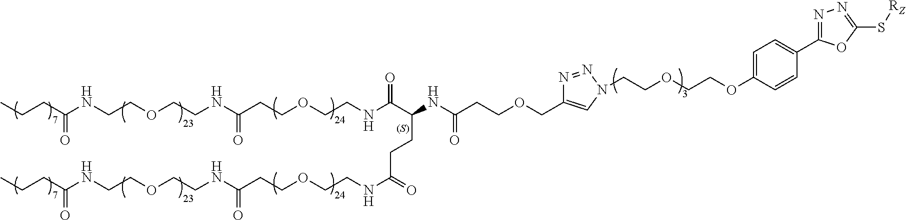

LP 107b

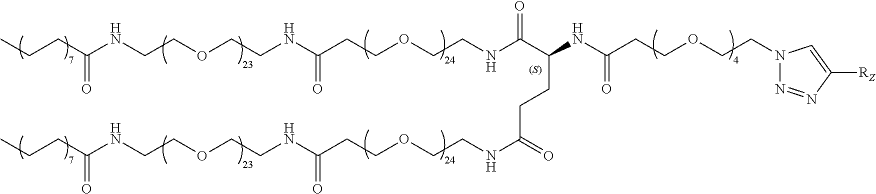

LP 108b

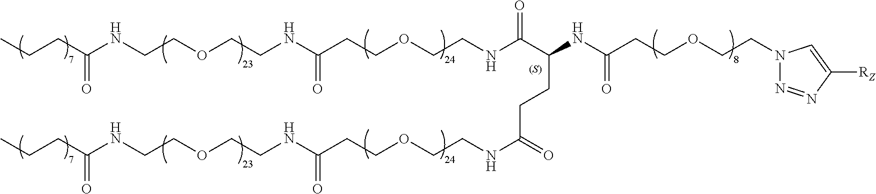

LP 109b

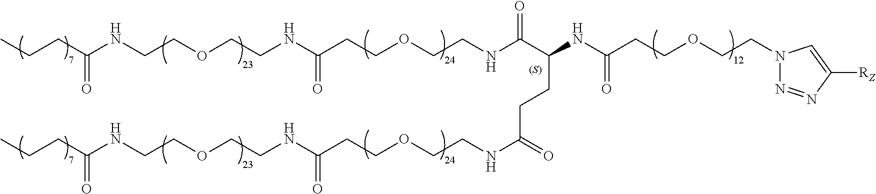

LP 110b

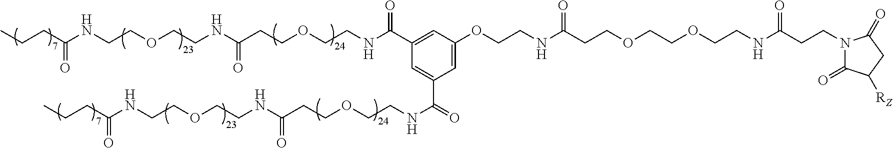

LP 111b

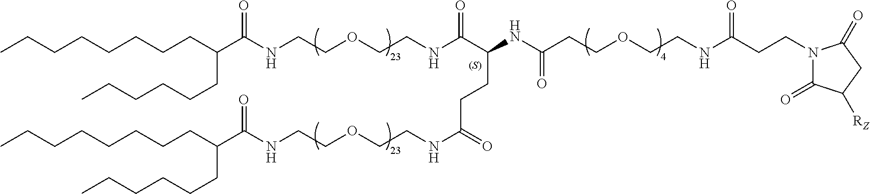

LP 124b

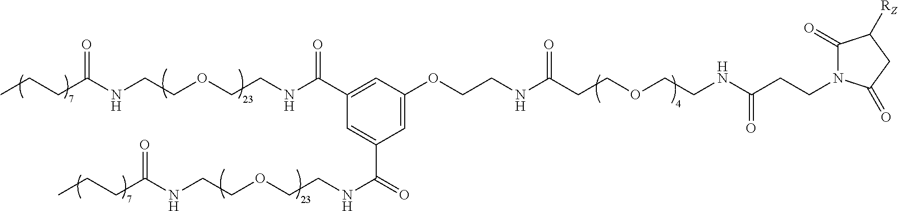

LP 130b

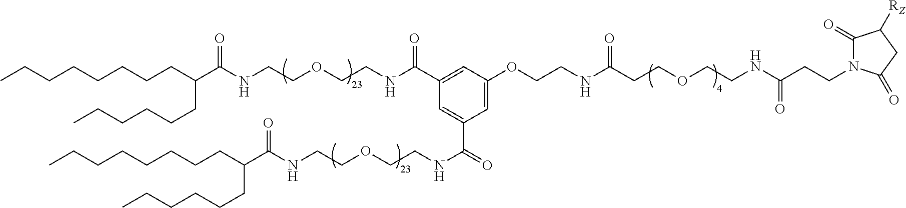

LP 143b

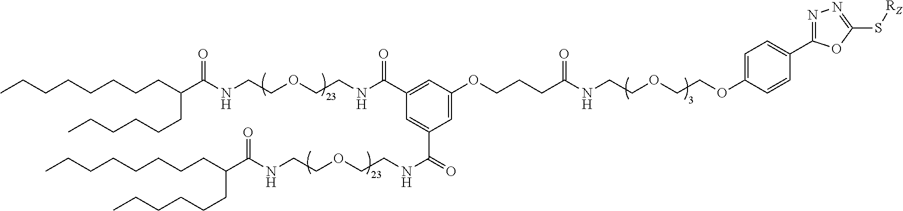

LP 210b

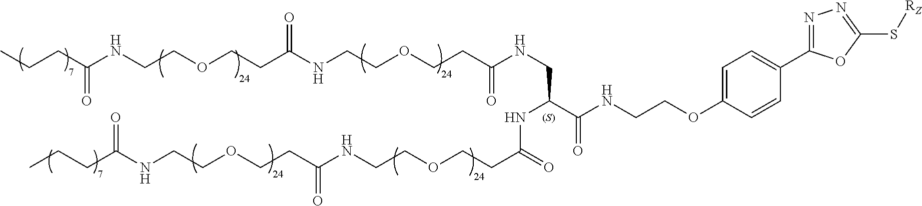

LP 217b

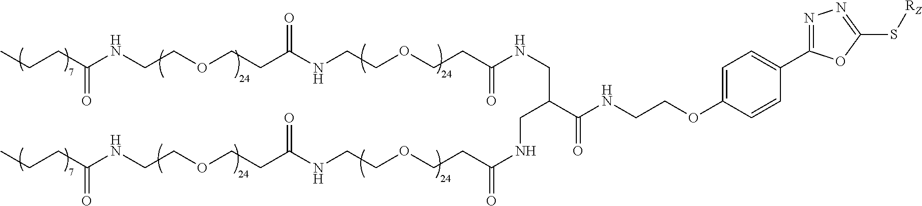

LP 220b

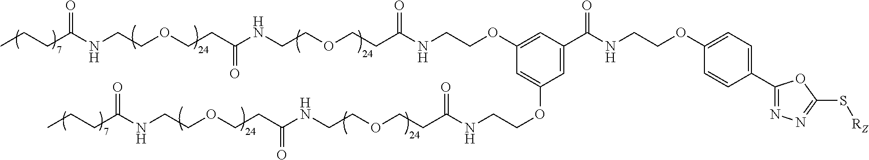

LP 221b

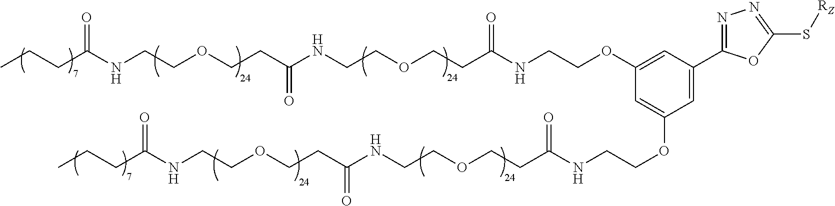

LP 223b

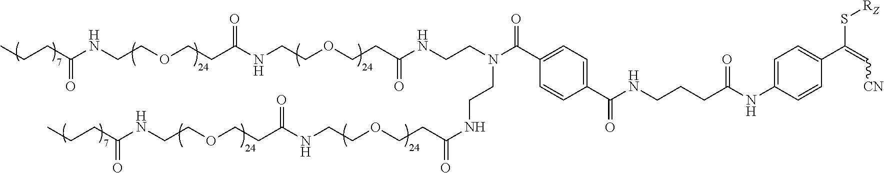

LP 224b

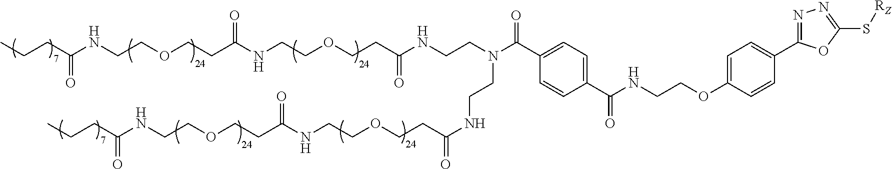

LP 225b

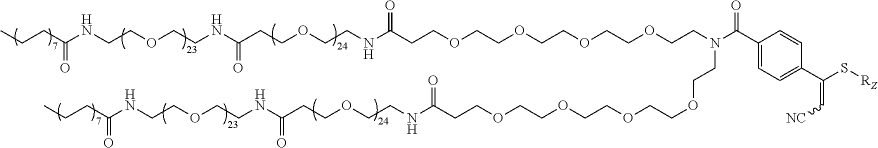

LP 226b

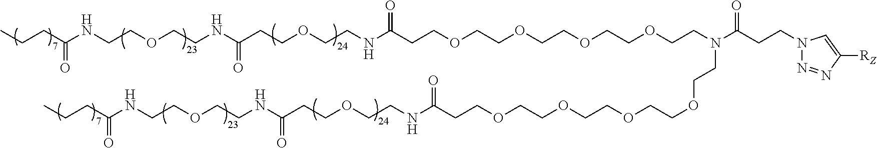

LP 238b

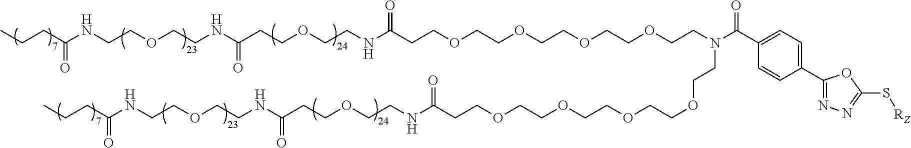

LP 240b

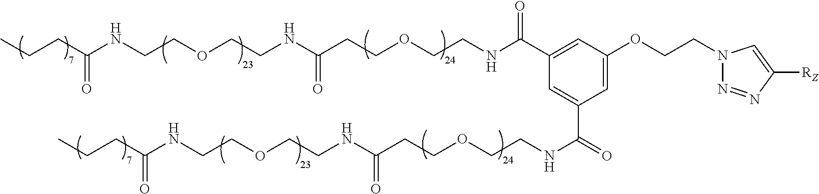

LP 246b

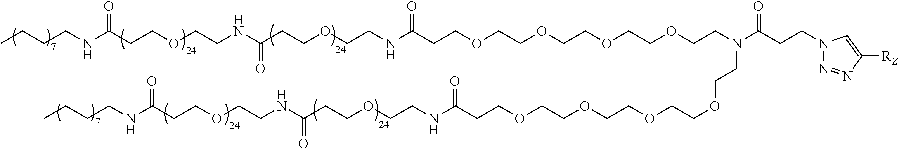

LP 247b

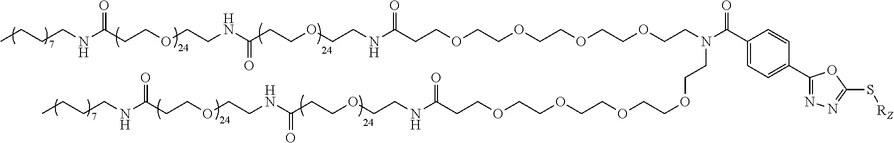

LP 339b

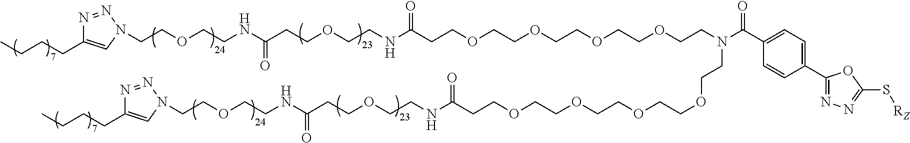

LP 340b

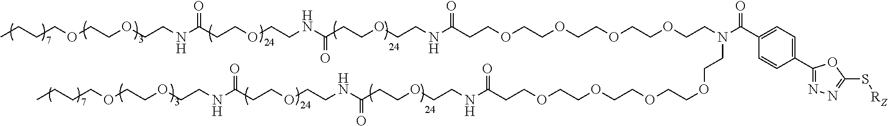

LP 357b

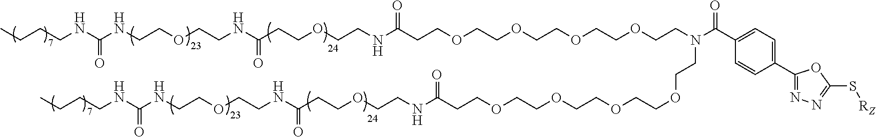

LP 358b

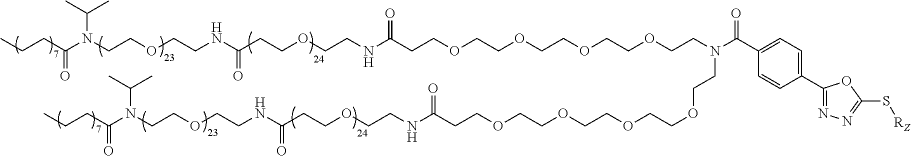

or a pharmaceutically acceptable salt of any of these compounds, wherein each R_{Z} comprises an oligonucleotide-based agent.

In another aspect of the invention, a compound is selected from the group consisting of the compounds identified in Table 17, or a pharmaceutically acceptable salt thereof.

TABLE 17
Example compounds of the present invention (compound number appears before structure).

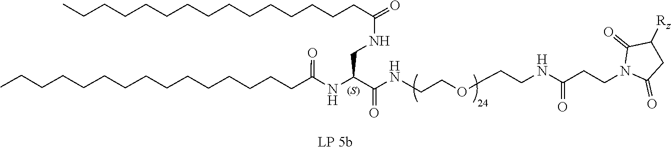

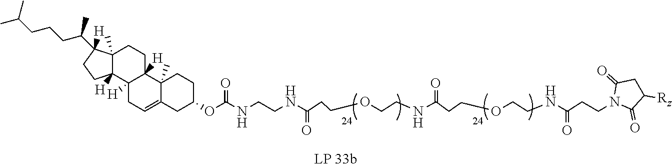

or a pharmaceutically acceptable salt of any of these compounds, wherein R_{Z} comprises an oligonucleotide-based agent.

Another aspect of the invention provides a compound of Formula (V):

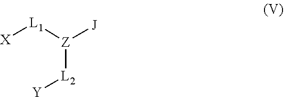

or a pharmaceutically acceptable salt thereof, wherein Z, L₁, L₂, X, and Y are as defined for any embodiments of the compound of Formula (I), (Ia), (Ib), (Ib1), or (Ic); J is L_{A5}-R_{X}; L_{A5} is a bond or a bivalent moiety connecting R_{X} to Z: and R_{X} is a reactive moiety for conjugation with an oligonucleotide-based agent.

In some embodiments, J is L_{A5}-R_{X}; L_{A5} is a bond or a bivalent moiety connecting R_{X} to Z; R_{X} is a reactive moiety for conjugation with an oligonucleotide-based agent; Z is CH, phenyl, or N; L₁ and L₂ are each independently linkers comprising at least about 5 PEG units; and X and Y are each independently lipids comprising from about 10 to about 50 carbon atoms.

In some embodiments, L_{A5} is L_{A} as defined in any embodiments of the compound of Formula (I), (Ia), (Ib), (Ib1), or (Ic). In some embodiments, L_{A5} is selected from the group consisting of the moieties identified in Table 18.

TABLE 18
Example L_{A5} moieties of the present invention.

| Name | Structure |
|---|---|
| Tether 1-5 | 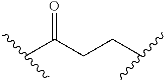 [SMILES: *CCC(*)=O] |
| Tether 2-5 | 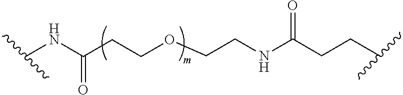 |
| Tether 3-5 | 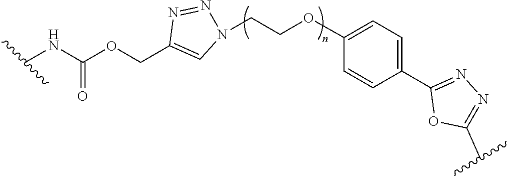 |
| Tether 4-5 | 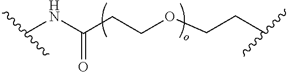 |
| Tether 5-5 | 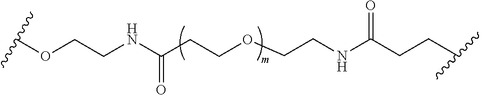 |
| Tether 6-5 | 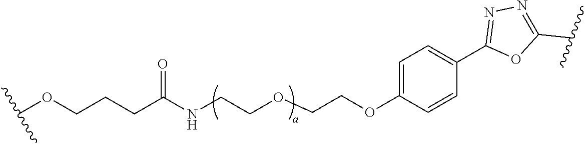 |
| Tether 7-5 | 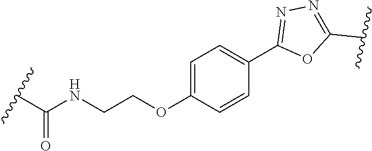 [SMILES: *C(=O)NCCOc1ccc(-c2nnc(*)o2)cc1] |
| Tether 8-5 | 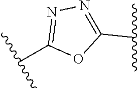 [SMILES: *c1nnc(*)o1] |
| Tether 9-5 | 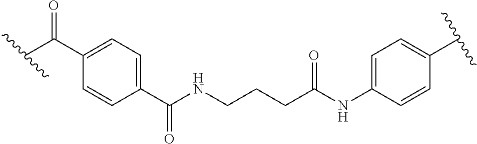 [SMILES: *C(=O)c1ccc(C(=O)NCCCC(=O)Nc2ccc(*)cc2)cc1] |
| Tether 10-5 | 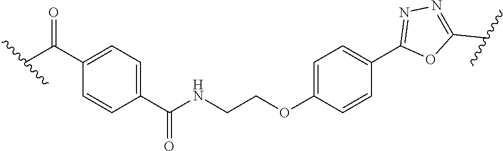 [SMILES: *C(=O)c1ccc(C(=O)NCCOc2ccc(-c3nnc(*)o3)cc2)cc1] |
| Tether 11-5 | 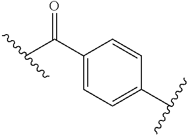 [SMILES: *C(=O)c1ccc(*)cc1] |
| Tether 12-5 | 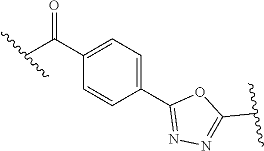 [SMILES: *C(=O)c1ccc(-c2nnc(*)o2)cc1] |
| Tether 13-5 | 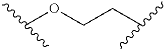 [SMILES: *CCO*] |

wherein each of m, n, o, and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and wherein each [symbol] indicates a point of connection to Z or R_{X}.

In some embodiments, each m is independently 1, 2, 3, 4, 5, 6, 7, 8, 9, 10, 20, 21, 22, 23, or 25; each n is independently 2, 3, 4, or 5; each a is independently 2, 3, or 4; and each o is independently 2, 3, 4, 5, 6, 7, 8, 9, 10, 11, 12, or 13.

In some embodiments, each m is independently 2, 4, 8, or 24. In some embodiments, each n is 4. In some embodiments, each o is independently 4, 8, or 12. In some embodiments, each a is 3.

In some embodiments, R_{X} is selected from the group consisting of

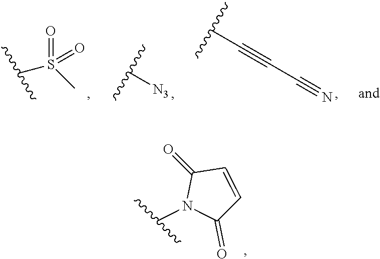

wherein each [symbol] indicates a point of connection to L_{A5}. In some embodiments, R_{X} is

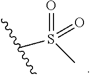 [SMILES: *S(C)(=O)=O]

In some embodiments, R_{X}

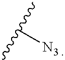 [SMILES: *N=[N+]=[N-]]

In some embodiments, R_{X} is

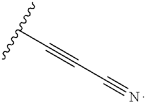 [SMILES: *C#CC#N]

In some embodiments, R_{X} is

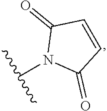 [SMILES: *N1C(=O)C=CC1=O]

In some embodiments, J is selected from the group consisting of the moieties identified in Table 19.

TABLE 19
Example J moieties of the present invention.
Structure

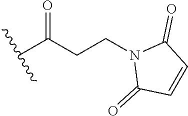 [SMILES: *C(=O)CCN1C(=O)C=CC1=O]

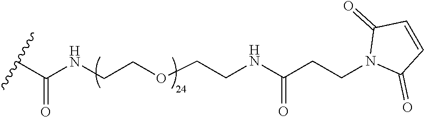

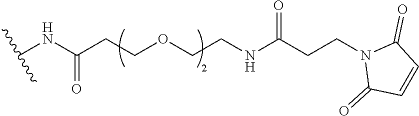

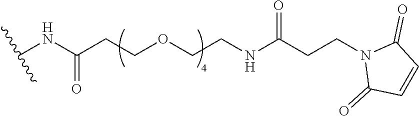

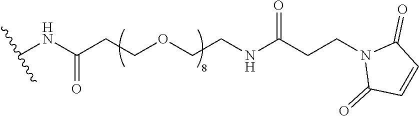

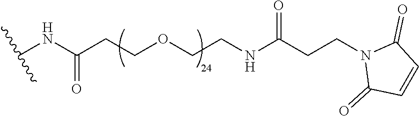

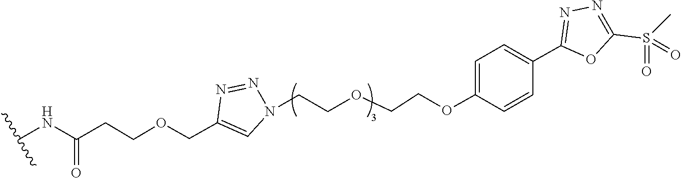

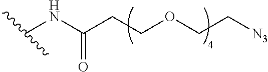

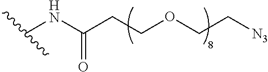

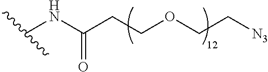

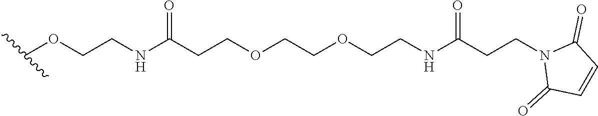 [SMILES: *OCCNC(=O)CCOCCOCCNC(=O)CCN1C(=O)C=CC1=O]

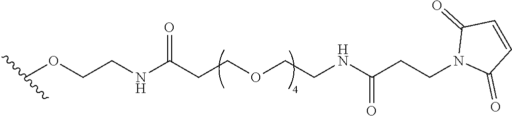

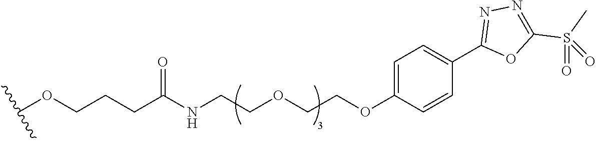

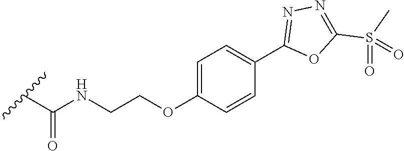 [SMILES: *C(=O)NCCOc1ccc(-c2nnc(S(C)(=O)=O)o2)cc1]

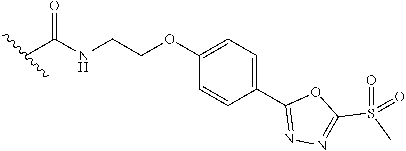 [SMILES: *C(=O)NCCOc1ccc(-c2nnc(S(C)(=O)=O)o2)cc1]

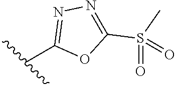 [SMILES: *c1nnc(S(C)(=O)=O)o1]

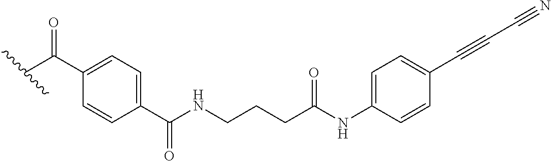 [SMILES: *C(=O)c1ccc(C(=O)NCCCC(=O)Nc2ccc(C#CC#N)cc2)cc1]

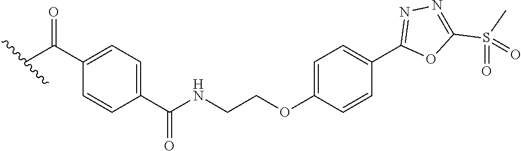 [SMILES: *C(=O)c1ccc(C(=O)NCCOc2ccc(-c3nnc(S(C)(=O)=O)o3)cc2)cc1]

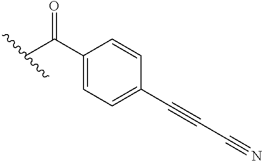 [SMILES: *C(=O)c1ccc(C#CC#N)cc1]

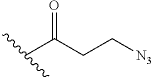 [SMILES: *C(=O)CCN=[N+]=[N-]]

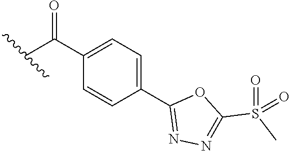 [SMILES: *C(=O)c1ccc(-c2nnc(S(C)(=O)=O)o2)cc1]

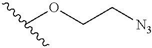 [SMILES: *OCCN=[N+]=[N-]]

wherein each [symbol] indicates a point of connection to Z.

Another aspect of the present invention provides a compound of Formula (Va):

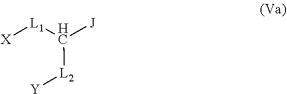

or a pharmaceutically acceptable salt thereof, wherein J, L₁, L₂, X, and Y are as defined in any of the embodiments of the compound of Formula (V).

In some embodiments, X and Y are each independently selected from the group consisting of Lipid 3, Lipid 4, Lipid, 5, Lipid 6, Lipid 7, Lipid 10, Lipid 12, and Lipid 19 as set forth in Table 3 as set forth in Table 3, wherein each [symbol] indicates a point of connection to L₁ or L₂.

In some embodiments, each of L₁ and L₂ are independently selected from the group consisting of Linker 2, Linker 3, Linker 4, and Linker 5 as set forth in Table 1, wherein each [symbol] indicates a point of connection to X, Y, or CH of Formula (Va). In some embodiments, each p is 23. In some embodiments, each q is 24.

In some embodiments, LAS is selected from the group consisting of Tether 2-5, Tether 3-5, and Tether 4-5 as set forth in Table 18, wherein each [symbol] indicates a point of connection to R_{X} or CH of Formula (Va). In some embodiments, m is 2, 4, 8, or 24. In some embodiments, n is 4. In some embodiments, o is 4, 8, or 12.

In some embodiments each of L₁ and L₂ is independently selected from the group consisting of

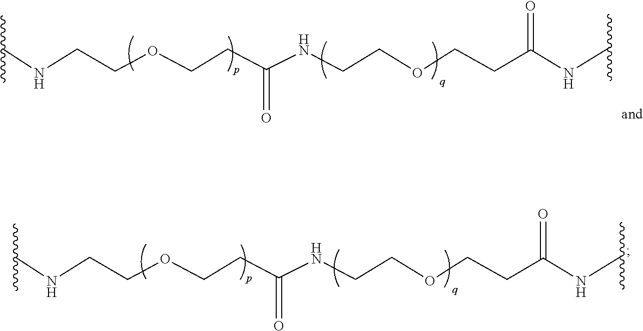

wherein each p is independently 20, 21, 22, 23, 24, or 25; each q is independently 20, 21, 22, 23, 24, or 25; and each [symbol] indicates a point of connection to X, Y, or CH of Formula (Va). In some embodiments, each p is 24. In some embodiments, each q is 24.

In some embodiments, L_{A5} is

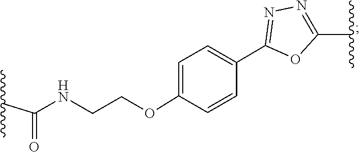 [SMILES: *C(=O)NCCOc1ccc(-c2nnc(*)o2)cc1]

wherein each [symbol] indicates a point of connection to R_{X} or CH of Formula (Va).

In some embodiments, each of X and Y is

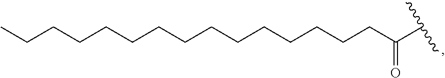 [SMILES: *C(=O)CCCCCCCCCCCCCCC]

wherein [symbol] indicates a point of connection to the L₁ or L₂.

In some embodiments, the compound of Formula (Va) is selected from the group consisting of LP210-p or LP 217-p as set forth in Table 20, or a pharmaceutically acceptable salt of any one of these compounds.

Another aspect of the present invention provides a compound of Formula (Vb):

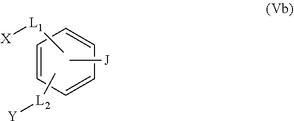

or a pharmaceutically acceptable salt thereof, wherein J, L₁, L₂, X, and Y are as defined in any of the embodiments of the compound of Formula (V) or (Va).

In some embodiments, X and Y are each independently selected from the group consisting of Lipid 3 and Lipid 19 as set forth in Table 3, wherein each [symbol] indicates a point of connection to L₁ or L₂. In some embodiments, X and Y are each Lipid 3. In some embodiments, X and Y are each Lipid 19.

In some embodiments, each of L₁ and L₂ is independently selected from the group consisting of Linker 3, Linker 5, and Linker 9 as set forth in Table 1, wherein each [symbol] indicates a point of connection to X, Y, or the phenyl ring of Formula (Vb). In some embodiments, p is 23 or 24. In some embodiments, q is 24.

In some embodiments, L_{A5} is selected from the group consisting of Tether 5-5, Tether, 6-5, Tether 7-5, Tether 8-5, and Tether 13-5 as set forth in Table 18, wherein each [symbol] indicates a point of connection to R_{X} or the phenyl ring of Formula (Vb). In some embodiments, m is 2 or 4. In some embodiments, a is 3.

Another aspect of the present invention provides a compound of Formula (Vb1):

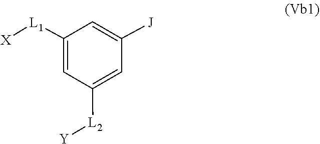

or a pharmaceutically acceptable salt thereof, wherein J, L₁, L₂, X, and Y are as defined in any of the embodiments of the compound of Formula (V), (Va), or (Vb).

Another aspect of the present invention provides a compound of Formula (Vc):

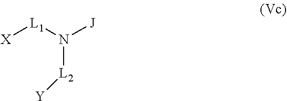

or a pharmaceutically acceptable salt thereof, wherein J, L₁, L₂, X, and Y are as defined in any of the embodiments of the compound of Formula (V), (Va), (Vb), or (Vb1).

In some embodiments, X and Y are each independently selected from the group consisting of Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, and Lipid 24 as set forth in Table 3, wherein each [symbol] indicates a point of connection to L₁ and L₂. In some embodiments, each of X and Y is Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, or Lipid 24.

In some embodiments, each of L₁ and L₂ is independently selected from the group consisting of Linker 1, Linker 6, Linker 10, Linker 11, and Linker 12 as set forth in Table 1, wherein each [symbol] indicates a point of connection to X, Y, or N of Formula (Vc). In some embodiments, p is independently 23 or 24. In some embodiments, q is 24. In some embodiments, r is 4.

In some embodiments, L_{A5} is selected from the group consisting of Tether 1-5, Tether 9-5, Tether 10-5, Tether 11-5, or Tether 12-5 as set forth in Table 18, wherein each [symbol] indicates a point of connection to R_{Z} or N of Formula (Vc).

Another aspect of the present invention provides a compound of Formula (Vd):

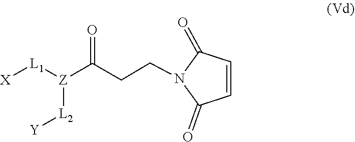

or a pharmaceutically acceptable salt thereof, wherein Z, L₁, L₂, X, and Y are as defined in any of the embodiments of the compound of Formula (V), (Va), (Vb) (Vb1), or (Vc).

Another aspect of the present invention provides a compound of Formula (Ve):

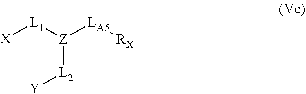

or a pharmaceutically acceptable salt thereof, wherein Z, L₁, L₂, R_{X}, L_{A5}, X, and Y are as defined in any of the embodiments of the compound of Formula (V), (Va), (Vb) (Vb1), (Vc) or (Vd).

Another aspect of the present invention provides a compound of Formula (Ve1):

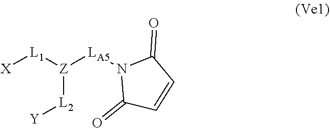

or a pharmaceutically acceptable salt thereof, wherein Z, L₁, L₂, LAS, X, and Y are as defined in any of the embodiments of the compound of Formula (V), (Va), (Vb) (Vb1), (Vc), (Vd), or (Ve).

Another aspect of the present invention provides a compound of Formula (Ve2):

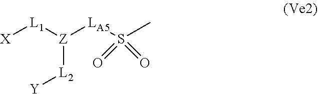

or a pharmaceutically acceptable salt thereof, wherein Z, L₁, L₂, LAS, X, and Y are as defined in any of the embodiments of the compound of Formula (V), (Va), (Vb) (Vb1), (Vc), (Vd), (Ve), or (Ve1).

Another aspect of the present invention provides a compound of Formula (Ve3):

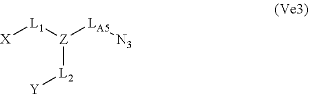

or a pharmaceutically acceptable salt thereof, wherein Z, L₁, L₂, LAS, X, and Y are as defined in any of the embodiments of the compound of Formula (V), (Va), (Vb) (Vb1), (Vc), (Vd), (Ve), (Ve1), or (Ve2).

Another aspect of the present invention provides a compound of Formula (Ve4):

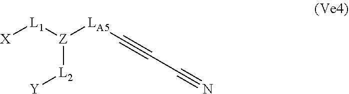

or a pharmaceutically acceptable salt thereof, wherein Z, L₁, L₂, LAS, X, and Y are as defined in any of the embodiments of the compound of Formula (V), (Va), (Vb) (Vb1), (Vc), (Vd), (Ve), (Ve1), (Ve2), or (Ve3).

In another aspect of the invention, a compound is selected from the group consisting of the compounds identified in Table 20, or a pharmaceutically acceptable salt thereof.

TABLE 20
Example compounds of the present invention (compound number appears before structure).

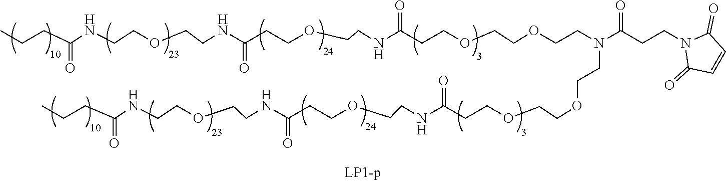

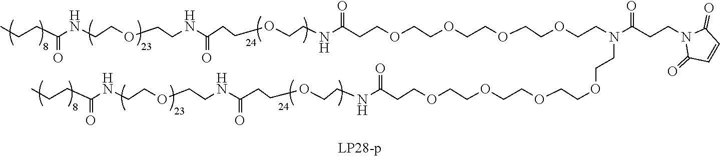

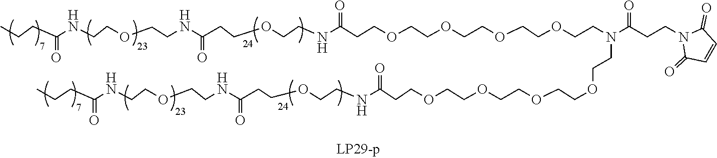

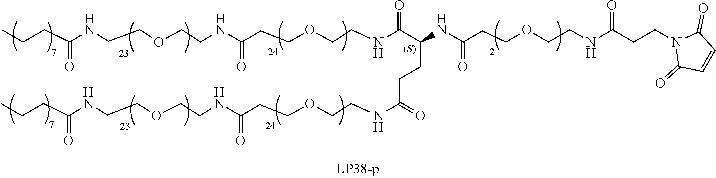

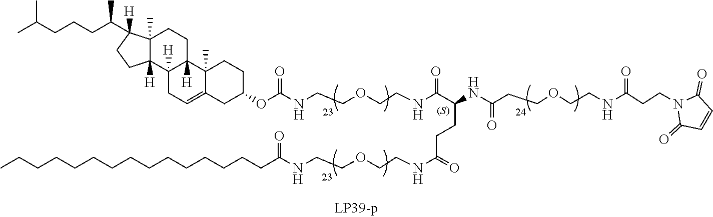

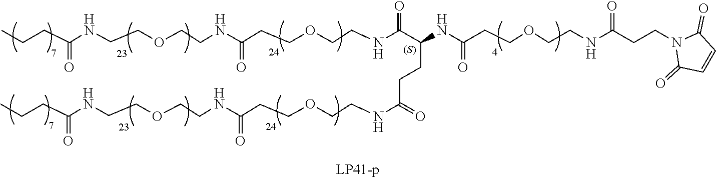

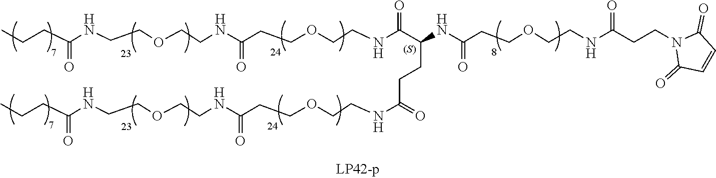

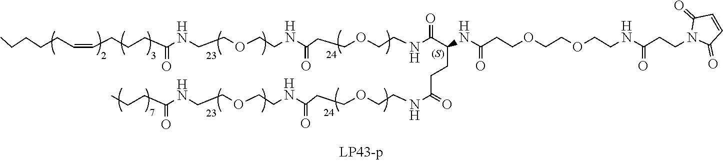

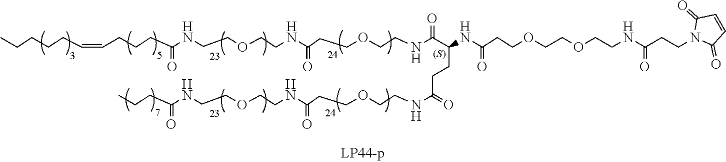

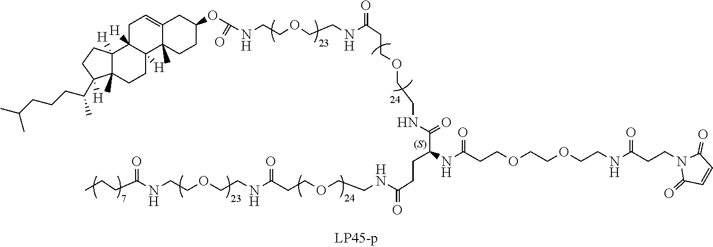

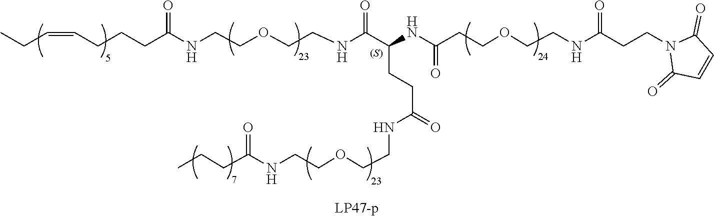

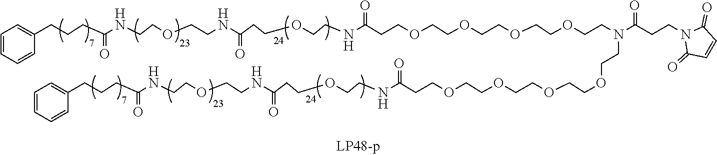

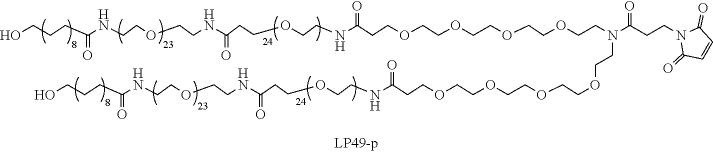

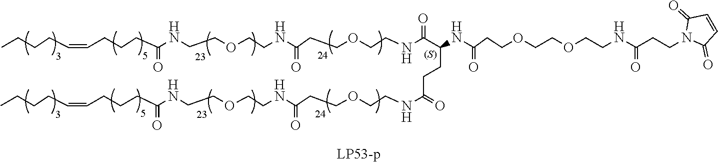

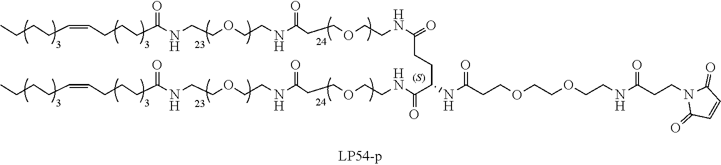

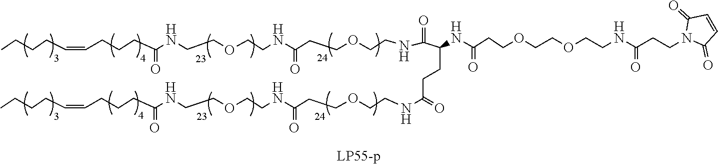

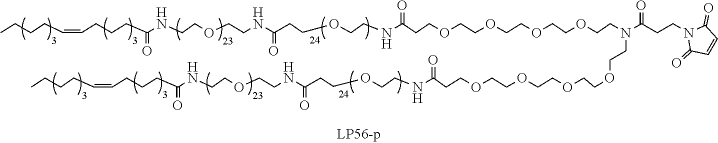

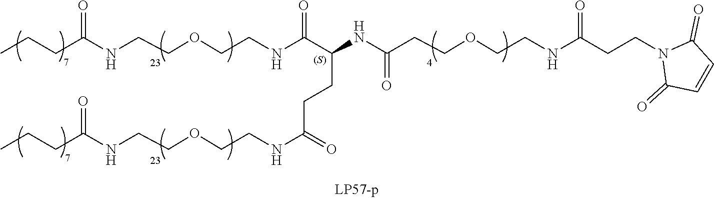

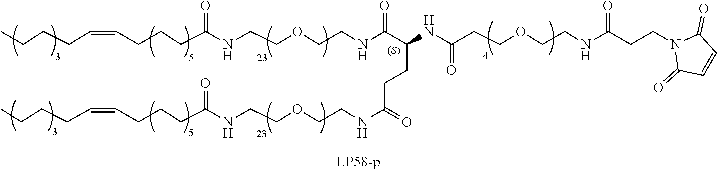

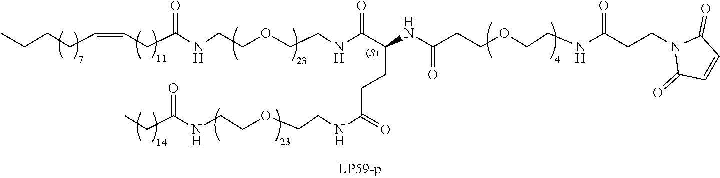

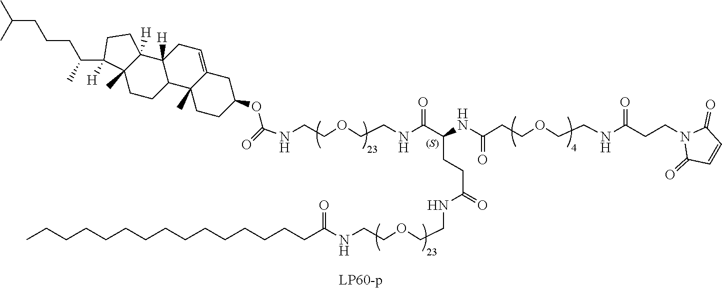

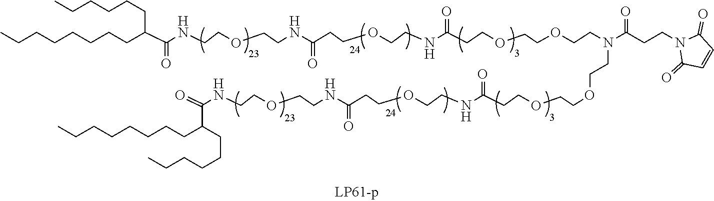

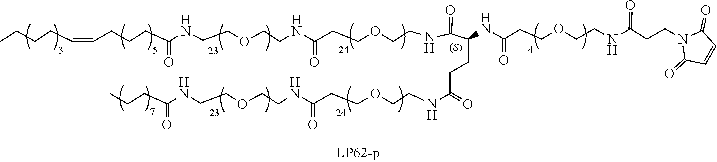

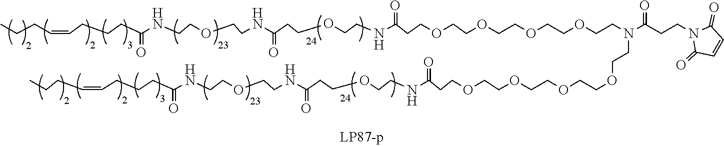

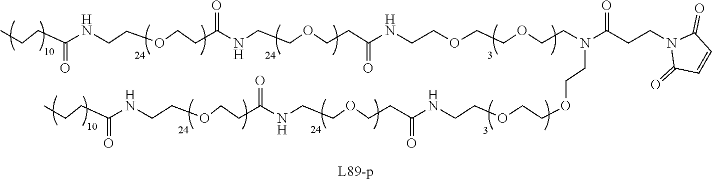

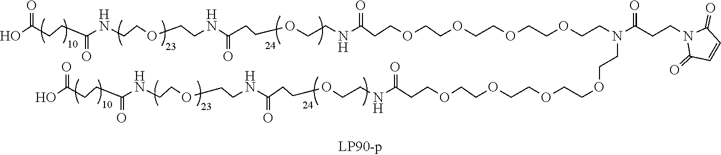

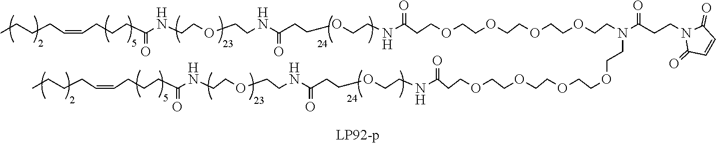

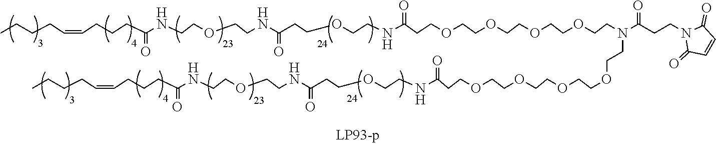

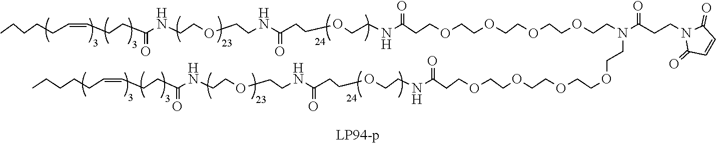

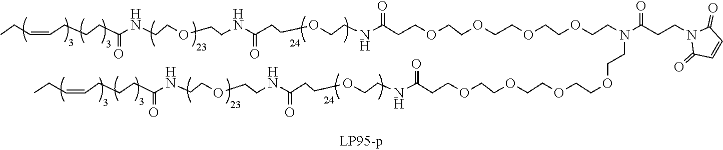

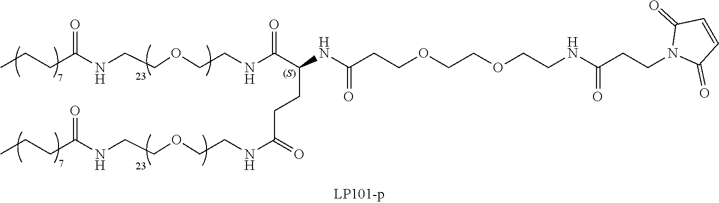

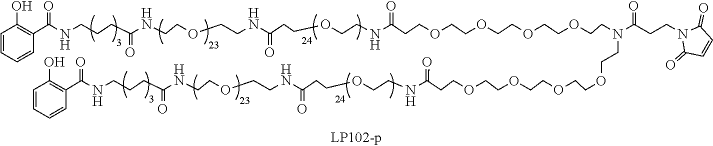

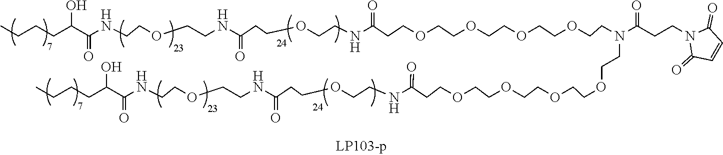

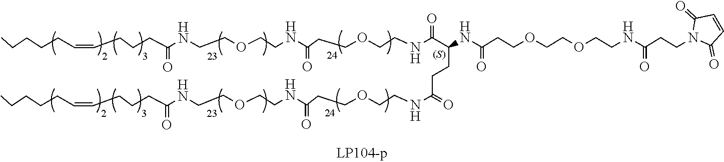

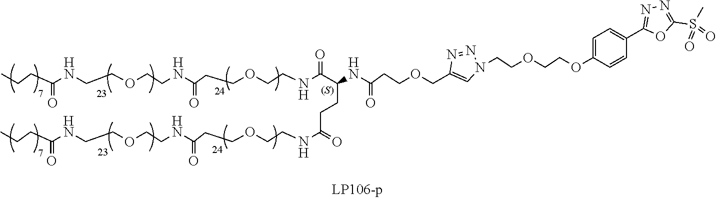

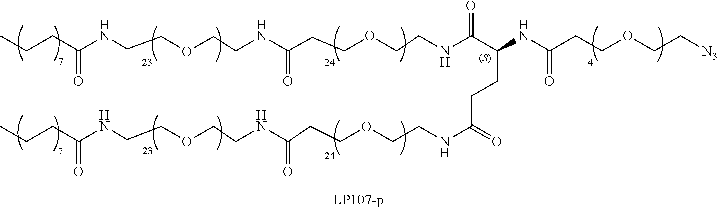

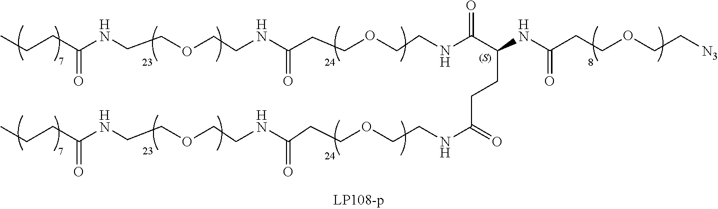

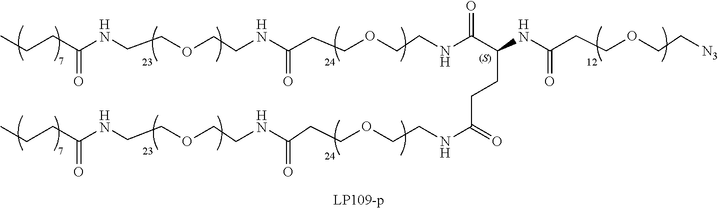

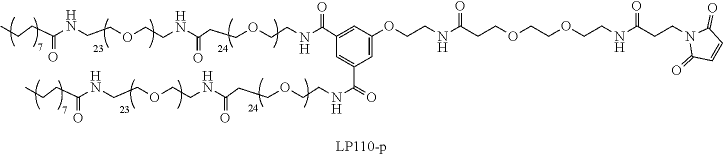

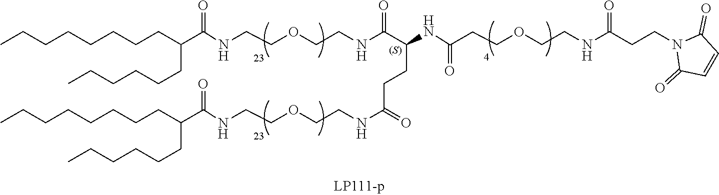

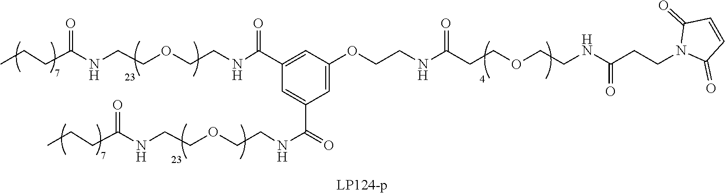

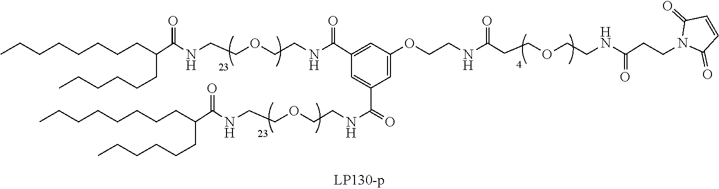

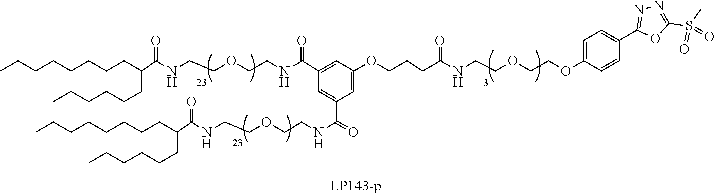

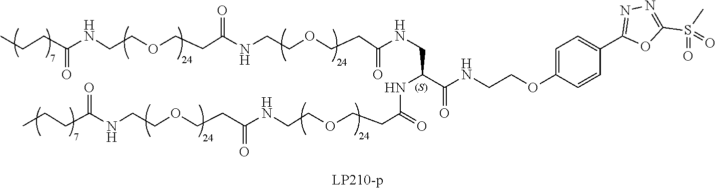

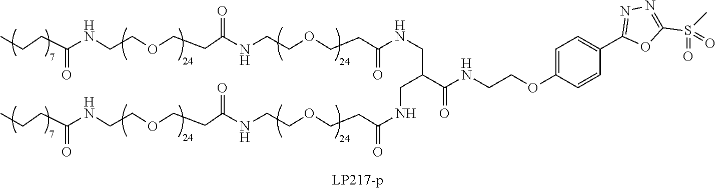

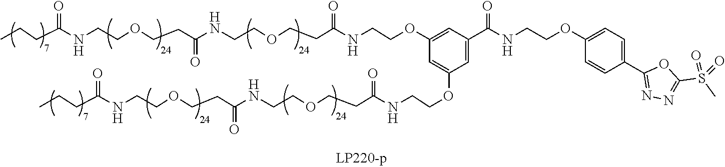

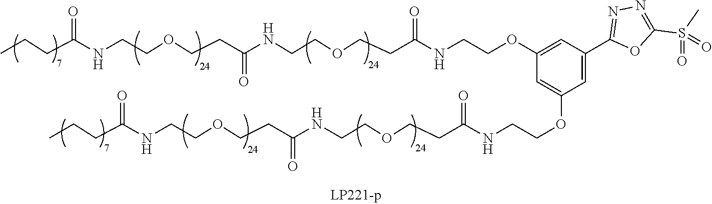

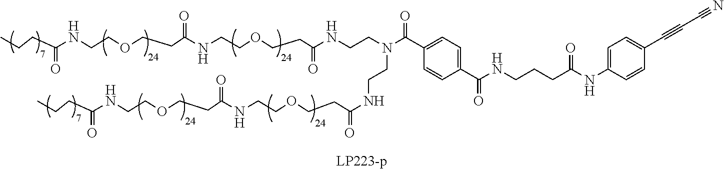

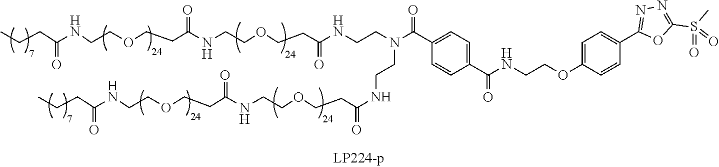

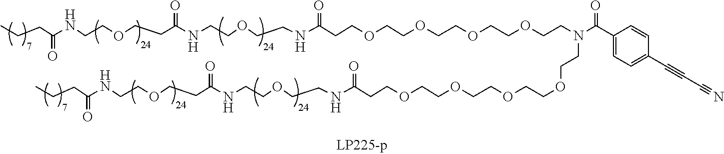

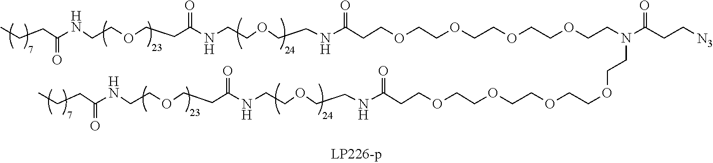

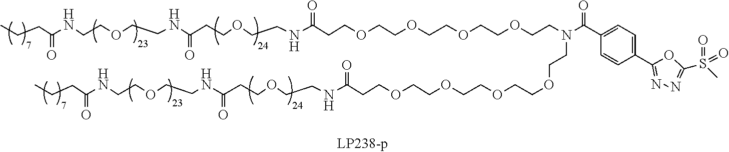

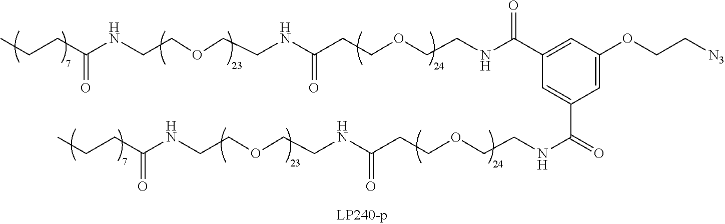

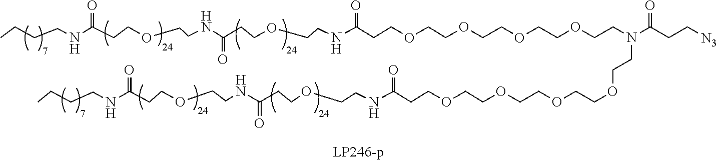

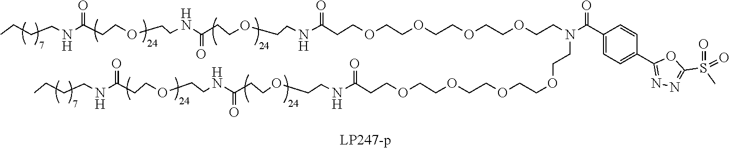

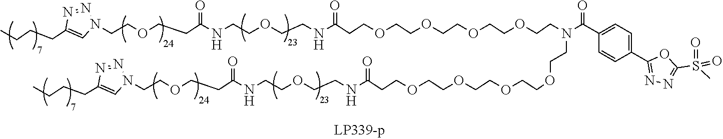

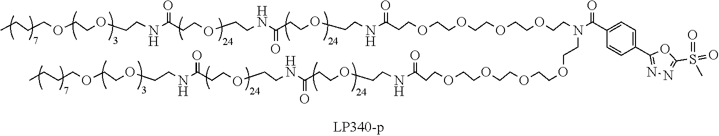

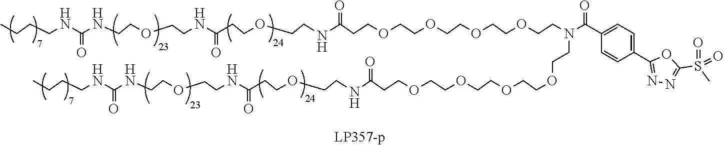

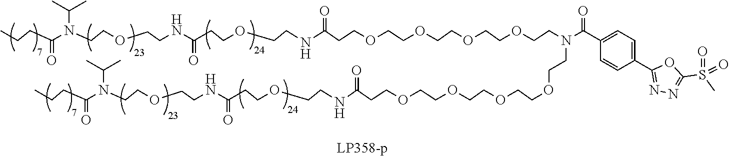

or a pharmaceutically acceptable salt of any of these compounds.

In another aspect of the invention, a compound is selected from the group consisting of the compounds identified in Table 21, or a pharmaceutically acceptable salt thereof.

TABLE 21
Example compounds of the present invention (compound name appears before structure).

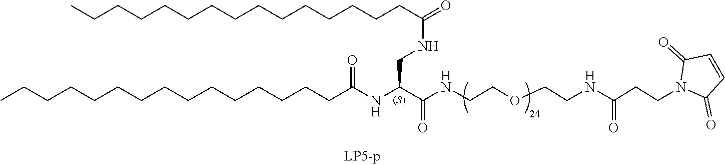

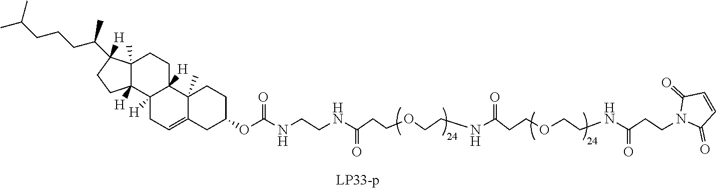

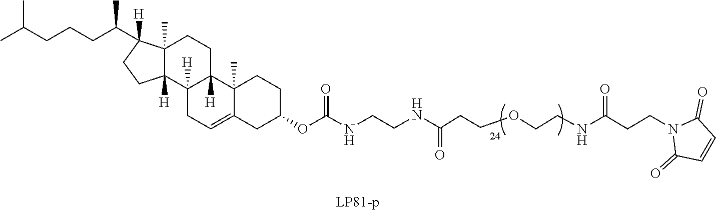

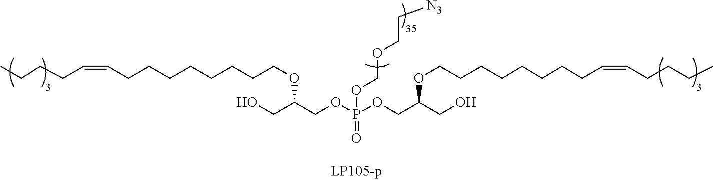

or a pharmaceutically acceptable salt of any of these compounds.

Another aspect of the present invention provides a process for making compounds of Formula (I):

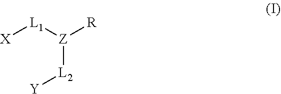

or a pharmaceutically acceptable salt thereof.

The method comprises conjugating an oligonucleotide-based agent comprising a first reactive moiety with a compound comprising a lipid and a second reactive moiety to form a compound of Formula (I).

In some embodiments, the first reactive moiety is selected from the group consisting of a disulfide and a propargyl group. In some embodiments, the first reactive moiety is a disulfide. In some embodiments, the first reactive moiety is a propargyl group.

In some embodiments, the second reactive moiety is selected from the group consisting of maleimide, sulfone, azide, and alkyne. In some embodiments, the second reactive moiety is a maleimide. In some embodiments, the second reactive moiety is a sulfone. In some embodiments, the second reactive moiety is an azide. In some embodiments, the second reactive moiety is an alkyne.

Compounds of Formula (V), (Va), (Vb), (Vb1), (Vc), (Vd), (Ve), (Ve1), (Ve2), (Ve3), or (Ve4) described herein may be referred to as “pharmacokinetic and/or pharmacodynamic modulator precursors” (hereinafter, “PK/PD modulator precursors”). It will also be appreciated that portions of compounds of Formula (I), (Ia), (Ib), (Ib1), (Ic), (Id), (II), (III), (IIIa), (IIIb), (IV), or (IVa) described herein may be referred to as “pharmacokinetic and/or pharmacodynamic modulators” (hereinafter, “PK/PD modulators”). When used to refer to a portion of a compound of Formula (I), (Ia), (Ib), (Ib1), (Ic), (Id), (II), (III), (IIIa), (IIIb), (IV), or (IVa) the term “PK/PD modulator” refers to the portion of the compound excluding R_{Z} (i.e., the oligonucleotide-based agent).

A PK/PD modulator is linked to an oligonucleotide-based agent, such as an RNAi agent, to facilitate delivery of the RNAi agent to the desired cells or tissues. PK/PD modulator precursors can be synthetized having reactive moieties, such as maleimide or azido groups, that readily facilitate linkage to one or more linking groups on an RNAi agent. Chemical reaction syntheses to link such PK/PD modulator precursors to RNAi agents are generally known in the art. The terms “PK/PD modulator” and “lipid PK/PD modulator” may be used interchangeably herein.

PK/PD modulator precursors selected from the group consisting of LP1-p, LP5-p, LP28-p, LP29-p, LP33-p, LP38-p, LP39-p, LP41-p, LP42-p, LP43-p, LP44-p, LP45-p, LP47-p, LP48-p, LP49-p, LP53-p, LP54-p, LP55-p, LP56-p, LP57-p, LP58-p, LP59-p, LP60-p, LP61-p, LP62-p, LP81-p, LP87-p, LP89-p, LP90-p, LP92-p, LP93-p, LP94-p, LP95-p, LP101-p, LP102-p, LP103-p, LP104-p, LP105-p, LP106-p, LP107-p, LP108-p, LP109-p, LP110-p, LP111-p, LP124-p, LP130-p, LP143-p, LP210-p, LP217-p, LP220-p, LP221-p, LP223-p, LP224-p, LP225-p, LP226-p, LP238-p, LP240-p, LP246-p, LP247-p, LP339-p, LP340-p, LP357-p, and LP358-p can be used as starting materials to link to RNAi agents. The PK/PD modulator precursors may be covalently attached to an RNAi agent using any known method in the art. For example, in some embodiments, maleimide-containing PK/PD modulator precursors may be reacted with a disulfide-containing moiety on the 3′ end of the sense strand.

In some embodiments, one or more PK/PD modulators may be conjugated to RNAi agents described herein. In some embodiments, one, two, three, four, five, six, seven or more PK/PD modulators may be conjugated to RNAi agents described herein.

PK/PD modulator precursors may be conjugated to RNAi agents using any known method in the art. In some embodiments, PK/PD modulator precursors comprising a maleimide moiety may be reacted with RNAi agents comprising a disulfide linkage to form a compound comprising a PK/PD modulator conjugated to an RNAi agent. The disulfide may be reduced, and added to a maleimide by way of a Michael-Addition reaction. An example reaction scheme is shown below:

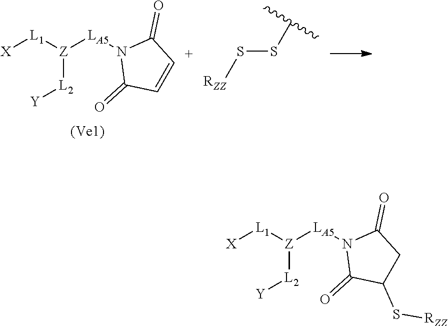

wherein R_{ZZ} comprises an RNAi agent, and [symbol] indicates a point of connection to any suitable group known in the art. In some instances of the reaction scheme above,

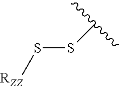

is attached to an alkyl group such as hexyl (C₆H₁₃).

In some embodiments, PK/PD modulator precursors may comprise a sulfone moiety and may react with a disulfide. An example reaction scheme is shown below:

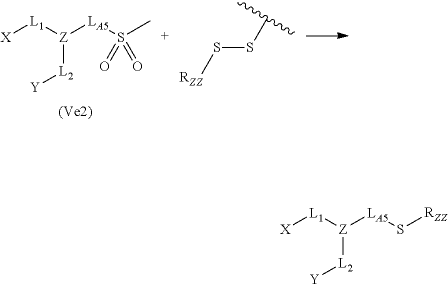

wherein R_{ZZ} comprises an RNAi agent, and [symbol] indicates a point of connection to any suitable group known in the art. In some instances of the reaction scheme above,

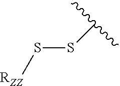

is attached to an alkyl group such as hexyl (C₆H₁₃).

In some embodiments, PK/PD modulator precursors may comprise an azide moiety and be reacted with an RNAi agent comprising an alkyne to form a compound comprising a PK/PD modulator conjugated to an RNAi agent according to the general reaction scheme below:

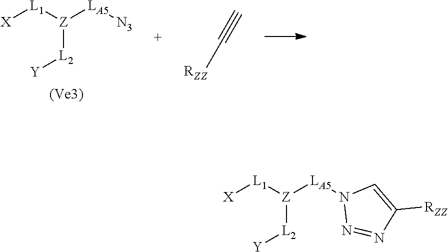

wherein R_{ZZ} comprises an RNAi agent.

In some embodiments, PK/PD modulator precursors may comprise an alkyne moiety and be reacted with an RNAi agent comprising a disulfide to form a compound comprising a PK/PD modulator conjugated to an RNAi agent according to the general reaction scheme below:

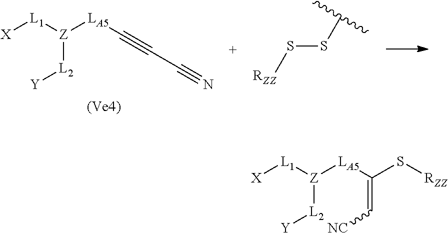

wherein R_{ZZ} comprises an RNAi agent, and [symbol] indicates a point of connection to any suitable group known in the art. In some instances of the reaction scheme above,

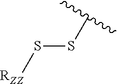

is attached to an alkyl group such as hexyl (C₆H₁₃).

In some embodiments, PK/PD modulators may be conjugated to the 5′ end of the sense or antisense strand, the 3′ end of the sense or antisense strand, or to an internal nucleotide of RNAi agents. In some embodiments, an RNAi agent is synthesized with a disulfide-containing moiety at the 3′ end of the sense strand, and a PK/PD modulator precursor may be conjugated to the 3′ end of the sense strand using any of the appropriate general synthetic schemes shown above.

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

As used herein, the term “lipid” refers to moieties and molecules that are soluble in nonpolar solvents. The term lipid includes amphiphilic molecules comprising a polar, water-soluble head group and a hydrophobic tail. Lipids can be of natural or synthetic origin. Non-limiting examples of lipids include fatty acids (e.g., saturated fatty acids, monounsaturated fatty acids, and polyunsaturated fatty acids), glycerolipids (e.g., monoacylglycerols, diacylglycerols, and triacylglycerols), phospholipids (e.g., phosphatidylethanolamine, phosphatidylcholine, and phosphatidylserine), sphingolipids (e.g., sphingomyelin), and cholesterol esters. As used herein, the term “saturated lipid” refers to lipids that are free of any unsaturation. As used herein, the term “unsaturated lipid” refers to lipids that comprise at least one (1) degree of unsaturation. As used herein, the term “branched lipid” refers to lipids comprising more than one linear chain, wherein each liner chain is covalently attached to at least one other linear chain. As used herein, the term “straight chain lipid” refers to lipids that are free of any branching.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a target mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the preventative treatment, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

As used herein, the term “linked” or “conjugated” when referring to the connection between two compounds or molecules means that two molecules are joined by a covalent bond or are associated via noncovalent bonds (e.g., hydrogen bonds or ionic bonds). In some examples, where the term “linked” or “conjugated” refers to the association between two molecules via noncovalent bonds, the association between the two different molecules has a K_{D} of less than 1×10⁻⁴ M (e.g., less than 1×10⁻⁵ M, less than 1×10⁻⁶ M, or less than 1×10⁻⁷ M) in physiologically acceptable buffer (e.g., buffered saline). Unless stated, the terms “linked” and “conjugated” as used herein may refer to the connection between a first compound and a second compound either with or without any intervening atoms or groups of atoms.

As used herein, a linking group is one or more atoms that connects one molecule or portion of a molecule to another to second molecule or second portion of a molecule. Similarly, as used in the art, the term scaffold is sometimes used interchangeably with a linking group. Linking groups may comprise any number of atoms or functional groups. In some embodiments, linking groups may not facilitate any biological or pharmaceutical response, and merely serve to link two biologically active molecules.

As used herein, the term “alkyl” refers to a saturated aliphatic hydrocarbon group containing 1-12 (e.g., 1-8, 1-6, 1-4, or 1-3) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl.

Unless stated otherwise, use of the symbol [symbol] as used herein means that any group or groups may be linked (or connected) thereto that is in accordance with the scope of the inventions described herein.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

Oligonucleotide-Based Agents, Including RNAi Agents

As used herein, an “oligonucleotide-based agent” is a nucleotide sequence containing about 10-50 (e.g., 10 to 48, 10 to 46, 10 to 44, 10 to 42, 10 to 40, 10 to 38, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 50, 12 to 48, 12 to 46, 12 to 44, 12 to 42, 12 to 40, 12 to 38, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 50, 14 to 48, 14 to 46, 14 to 44, 14 to 42, 14 to 40, 14 to 38, 14 to 36, 14 to 34, 14 to 32, 14 to 30, 14 to 28, 14 to 26, 14 to 24, 14 to 22, 14 to 20, 14 to 18, 14 to 16, 16 to 50, 16 to 48, 16 to 46, 16 to 44, 16 to 42, 16 to 40, 16 to 38, 16 to 36, 16 to 34, 16 to 32, 16 to 30, 16 to 28, 16 to 26, 16 to 24, 16 to 22, 16 to 20, 16 to 18, 18 to 50, 18 to 48, 18 to 46, 18 to 44, 18 to 42, 18 to 40, 18 to 38, 18 to 36, 18 to 34, 18 to 32, 18 to 30, 18 to 28, 18 to 26, 18 to 24, 18 to 22, 18 to 20, 20 to 50, 20 to 48, 20 to 46, 20 to 44, 20 to 42, 20 to 40, 20 to 38, 20 to 36, 20 to 34, 20 to 32, 20 to 30, 20 to 28, 20 to 26, 20 to 24, 20 to 22, 22 to 50, 22 to 48, 22 to 46, 22 to 44, 22 to 42, 22 to 40, 22 to 38, 22 to 36, 22 to 34, 22 to 32, 22 to 30, 22 to 28, 22 to 26, 22 to 24, 24 to 50, 24 to 48, 24 to 46, 24 to 44, 24 to 42, 24 to 40, 24 to 38, 24 to 36, 24 to 34, 24 to 32, 24 to 30, 24 to 28, 24 to 26, 26 to 50, 26 to 48, 26 to 46, 26 to 44, 26 to 42, 26 to 40, 26 to 38, 26 to 36, 26 to 34, 26 to 32, 26 to 30, 26 to 28, 28 to 50, 28 to 48, 28 to 46, 28 to 44, 28 to 42, 28 to 40, 28 to 38, 28 to 36, 28 to 34, 28 to 32, to 28 to 30, 30 to 50, 30 to 48, 30 to 46, 30 to 44, 30 to 42, 30 to 40, 30 to 38, 30 to 36, 30 to 34, 30 to 32, 32 to 50, 32 to 48, 32 to 46, 32 to 44, 32 to 42, 32 to 40, 32 to 38, 32 to 36, 32 to 34, 34 to 50, 34 to 48, 34 to 46, 34 to 44, 34 to 42, 34 to 40, 34 to 38, 34 to 36, 36 to 50, 36 to 48, 36 to 46, 36 to 44, 36 to 42, 36 to 40, 36 to 38, 38 to 50, 38 to 48, 38 to 46, 38 to 44, 38 to 42, 38 to 40, 40 to 50, 40 to 48, 40 to 46, 40 to 44, 40 to 42, 42 to 50, 42 to 48, 42 to 46, 42 to 44, 44 to 50, 44 to 48, 44 to 46, 46 to 50, 46 to 48, or 48 to 50) nucleotides or nucleotide base pairs. In some embodiments, an oligonucleotide-based agent has a nucleobase sequence that is at least partially complementary to a coding sequence in an expressed target nucleic acid or target gene within a cell. In some embodiments, the oligonucleotide-based agents, upon delivery to a cell expressing a gene, are able to inhibit the expression of the underlying gene, and are referred to herein as “expression-inhibiting oligonucleotide-based agents.” The gene expression can be inhibited in vitro or in vivo.

“Oligonucleotide-based agents” include, but are not limited to: single-stranded oligonucleotides, single-stranded antisense oligonucleotides, short interfering RNAs (siRNAs), double-strand RNAs (dsRNA), micro RNAs (miRNAs), short hairpin RNAs (shRNA), ribozymes, interfering RNA molecules, and dicer substrates. In some embodiments, an oligonucleotide-based agent is a single-stranded oligonucleotide, such as an antisense oligonucleotide. In some embodiments, an oligonucleotide-based agent is a double-stranded oligonucleotide. In some embodiments, an oligonucleotide-based agent is a double-stranded oligonucleotide that is an RNAi agent.

In some embodiments, the oligonucleotide-based agent is/are an “RNAi agent,” which as defined herein is a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double-strand RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

Typically, RNAi agents can be comprised of at least a sense strand (also referred to as a passenger strand) that includes a first sequence, and an antisense strand (also referred to as a guide strand) that includes a second sequence. The length of an RNAi agent sense and antisense strands can each be 16 to 49 nucleotides in length. In some embodiments, the sense and antisense strands of an RNAi agent are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. The sense and antisense strands can be either the same length or different lengths. The RNAi agents include an antisense strand sequence that is at least partially complementary to a sequence in the target gene, and upon delivery to a cell expressing the target, an RNAi agent may inhibit the expression of one or more target genes in vivo or in vitro.

Oligonucleotide-based agents generally, and RNAi agents specifically, may be comprised of modified nucleotides and/or one or more non-phosphodiester linkages. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides, 2′-modified nucleotides, 3′ to 3′ linkages (inverted) nucleotides, non-natural base-comprising nucleotides, bridged nucleotides, peptide nucleic acids, 2′,3′-seco nucleotide mimics (unlocked nucleobase analogues, locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e. a nucleotide with a group other than a hydroxyl group at the 2′ position of the five-membered sugar ring) include, but are not limited to, 2′-O-methyl nucleotides, 2′-deoxy-2′-fluoro nucleotides, 2′-deoxy nucleotides, 2′-methoxyethyl (2′-O-2-methoxylethyl) nucleotides, 2′-amino nucleotides, and 2′-alkyl nucleotides.

Moreover, one or more nucleotides of an oligonucleotide-based agent, such as an RNAi agent, may be linked by non-standard linkages or backbones (i.e., modified internucleoside linkages or modified backbones). A modified internucleoside linkage may be a non-phosphate-containing covalent internucleoside linkage. Modified internucleoside linkages or backbones include, but are not limited to, 5′-phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′.

It is not necessary for all positions in a given compound to be uniformly modified. Conversely, more than one modification may be incorporated in a single oligonucleotide-based agent or even in a single nucleotide thereof.

The RNAi agent sense strands and antisense strands may be synthesized and/or modified by methods known in the art. Additional disclosures related to RNAi agents may be found, for example, in the disclosure of modifications may be found, for example, in International Patent Application No. PCT/US2017/045446 (WO2018027106) to Arrowhead Pharmaceuticals, Inc., which also is incorporated by reference herein in its entirety.

Modified Nucleotides

In some embodiments, an RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-OMen), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e., a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (also referred to herein as 2′-fluoro nucleotide, and represented herein as Nf), 2′-deoxy nucleotides (represented herein as dN), 2′-methoxyethyl (2′-O-2-methoxylethyl) nucleotides (also referred to herein as 2′-MOE, and represented herein as NM), 2′-amino nucleotides, and 2′-alkyl nucleotides. It is not necessary for all positions in a given compound to be uniformly modified. Conversely, more than one modification can be incorporated in a single target RNAi agent or even in a single nucleotide thereof. The target RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some embodiments, one or more nucleotides of an RNAi agent is an unmodified ribonucleotide.

Modified Internucleoside Linkages

In some embodiments, one or more nucleotides of an RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an RNAi agent can contain 1, 2, 3, 4, 5, or 6 phosphorothioate linkages, an antisense strand of an RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an RNAi agent can contain 1, 2, 3, or 4 phosphorothioate linkages, an antisense strand of an RNAi agent can contain 1, 2, 3, or 4 phosphorothioate linkages, or both the sense strand and the antisense strand independently can contain 1, 2, 3, or 4 phosphorothioate linkages.

In some embodiments, an RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, one phosphorothioate internucleoside linkage is at the 5′ end of the sense strand, and another phosphorothioate linkage is at the 3′ end of the sense strand. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, an RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, an RNAi agent contains at least three or four phosphorothioate internucleoside linkages in the antisense strand.

In some embodiments, an RNAi agent contains one or more modified nucleotides and one or more modified internucleoside linkages. In some embodiments, a 2′-modified nucleoside is combined with modified internucleoside linkage.

Targeting Ligands and Targeting Groups

In some embodiments, oligonucleotide-based agents may also be conjugated to a targeting ligand or targeting group to form a compound according to the instant invention. Targeting ligands or targeting groups enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers. In some embodiments, a targeting group comprises an integrin targeting ligand.

In some embodiments, a targeting ligand enhances the ability of the RNAi agent to bind to a particular cell receptor on a cell of interest. In some embodiments, the targeting ligands conjugated to RNAi agents described herein have affinity for integrin receptors. In some embodiments, a suitable targeting ligand for use with the RNAi agents disclosed herein has affinity for integrin alpha-v-beta 6.

In some embodiments, the RNAi agents described herein are conjugated to targeting groups. Targeting groups comprise two or more targeting ligands.

In some embodiments, an RNAi agent disclosed herein is linked to one or more integrin targeting ligands that include the following structure:

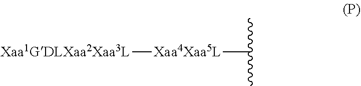

or a pharmaceutically acceptable salt thereof, wherein Xaa¹ is L-arginine optionally having an N-terminal cap,

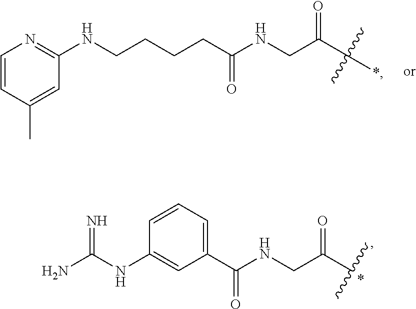

wherein [symbol] indicates a point of connection to G′; G′ is L-glycine or N-methyl-L-glycine; D is L-aspartic acid (L-aspartate); L is L-leucine; Xaa² is an L-α amino acid, an L-β amino acid, or an α,α-disubstituted amino acid; Xaa³ is an L-α amino acid, an L-β amino acid, or an α,α-disubstituted amino acid; Xaa⁴ is an L-α amino acid, an L-β amino acid, or an α,α-disubstituted amino acid; Xaa⁵ is an L-α amino acid, an L-β amino acid, or an α,α-disubstituted amino acid; and [symbol] indicates a point of connection to the RNAi agent.

In some embodiments, targeting ligands are conjugated to an RNAi agent using a “click” chemistry reaction. In some embodiments, RNAi agents are functionalized with one or more alkyne-containing groups, and targeting ligands include azide-containing groups. Upon reaction, azides and alkynes form triazoles. An example reaction scheme is shown below:

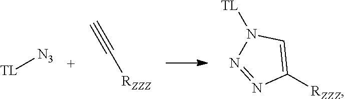

wherein TL comprises a targeting ligand, and R_{ZZZ} comprises an RNAi agent.

RNAi agents may comprise more than one targeting ligand. In some embodiments, RNAi agents comprise 1-20 targeting ligands. In some embodiments, RNAi agents comprise from 1, 2, 3, 4, 5, 6, 7, 8, 9, 10, 11, 12, 13, 14, 15, 16, 17, 18, or 19 targeting ligands to 2, 3, 4, 5, 6, 7, 8, 9, 10, 11, 12, 13, 14, 15, 16, 17, 18, 19 or 20 targeting ligands.

In some embodiments, RNAi agents described herein comprise a targeting group, which includes 2 or more targeting ligands. In some embodiments, a targeting group may be conjugated at the 5′ or 3′ end of the sense strand of an RNAi agent. In some embodiments, a targeting group may be conjugated to an internal nucleotide on an RNAi agent. In some embodiments, a targeting group may consist of two targeting ligands linked together, referred to as a “bidentate” targeting group. In some embodiments, a targeting group may consist of three targeting ligands linked together, referred to as a “tridentate” targeting group. In some embodiments, a targeting group may consist of four targeting ligands linked together, referred to as a “tetradentate” targeting group.

In some embodiments, RNAi agents may comprise both a targeting group conjugated to the 3′ or 5′ end of the sense strand, and additionally targeting ligands conjugated to internal nucleotides. In some embodiments a tridentate targeting group is conjugated to the 5′ end of the sense strand of an RNAi agent, and at least one targeting ligand is conjugated to an internal nucleotide of the sense strand. In further embodiments, a tridentate targeting group is conjugated to the 5′ end of the sense strand of an RNAi agent, and four targeting ligands are conjugated to internal nucleotides of the sense strand.

Linking Groups and Delivery Agents

In some embodiments, the oligonucleotide-based agent, such as RNAi agents described herein, contains or is conjugated to one or more non-nucleotide groups including, but not limited to a linking group or a delivery agent. The non-nucleotide group can enhance targeting, delivery, or attachment of the RNAi agent. Examples of linking groups are provided in Table 22. The non-nucleotide group can be covalently linked to the 3′ and/or 5′ end of either the sense strand and/or the antisense strand. In some embodiments, an RNAi agent contains a non-nucleotide group linked to the 3′ and/or 5′ end of the sense strand. In some embodiments, a non-nucleotide group is linked to the 5′ end of an RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

The RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the RNAi agents disclosed herein are synthesized having an NH₂-C₆ group at the 5′-terminus of the sense strand of the RNAi agent. The terminal amino group subsequently can be reacted to form a conjugate with, for example, a group that includes a compound having affinity for one or more integrins (i.e., and integrin targeting ligand) or a PK enhancer. In some embodiments, the RNAi agents disclosed herein are synthesized having one or more alkyne groups at the 5′-terminus of the sense strand of the RNAi agent. The terminal alkyne group(s) can subsequently be reacted to form a conjugate with, for example, a group that includes a targeting ligand.

In some embodiments, a targeting group comprises an integrin targeting ligand. In some embodiments, an integrin targeting ligand includes a compound that has affinity to integrin alpha-v-beta 6. The use of an integrin targeting ligands can facilitate cell-specific targeting to cells having the respective integrin on its respective surface, and binding of the integrin targeting ligand can facilitate entry of the RNAi agent, to which it is linked, into cells such as skeletal muscle cells. Targeting ligands, targeting groups, and/or PK/PD modulators can be attached to the 3′ and/or 5′ end of the RNAi agent, and/or to internal nucleotides on the RNAi agent, using methods generally known in the art. The preparation of targeting ligand and targeting groups, such as integrin αvβ6 is described in Example 3 below.

Some embodiments of the present disclosure include pharmaceutical compositions for delivering an RNAi agent to a skeletal muscle cell in vivo. Such pharmaceutical compositions can include, for example, an RNAi agent conjugated to a targeting group that comprises an integrin targeting ligand that has affinity for integrin αvβ6. In some embodiments, the targeting ligand is comprised of a compound having affinity for integrin αvβ6.

In some embodiments, the RNAi agent is synthesized having present a linking group, which can then facilitate covalent linkage of the RNAi agent to a targeting ligand, a targeting group, a PK/PD modulator, or another type of delivery agent. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: Alk-SMPT-C6, Alk-SS-C6, DBCO-TEG, Me-Alk-SS-C6, and C6-SS-Alk-Me, reactive groups such a primary amines and alkynes, alkyl groups, abasic residues/nucleotides, amino acids, trialkyne functionalized groups, ribitol, and/or PEG groups.

A linker or linking group is a connection between two atoms that links one chemical group (such as an RNAi agent) or segment of interest to another chemical group (such as a targeting ligand, targeting group, PK/PD modulator, or delivery agent) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, targeting groups are linked to the RNAi agents without the use of an additional linker. In some embodiments, the targeting group is designed to have a linker readily present to facilitate the linkage to an RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

RNAi agents whether modified or unmodified, may contain 3′ and/or 5′ targeting group(s), linking group(s), and/or may be conjugated with, or comprise, PK/PD modulator(s). Any of the RNAi agent sequences listed in Tables 3, 4, 8, 9, 14 and 15 or are otherwise described herein, which contain a 3′ or 5′ targeting ligand, targeting group, PK/PD modulator, or linking group, can alternatively contain no 3′ or 5′ targeting ligand, targeting group, linking group, or PK/PD modulator, or can contain a different 3′ or 5′ targeting ligand, targeting group, linking group, or PK/PD modulator including, but not limited to, those depicted in Table 22. Any of the RNAi agent duplexes listed in Table 24 whether modified or unmodified, can further comprise a targeting ligand, targeting group, linking group, or PK/PD modulator, and the targeting group or linking group can be attached to the 3′ or 5′ terminus of either the sense strand or the antisense strand of the RNAi agent duplex.

In some embodiments, a linking group may be conjugated synthetically to the 5′ or 3′ end of the sense strand of an RNAi agent described herein. In some embodiments, a linking group is conjugated synthetically to the 5′ end of the sense strand of an RNAi agent. In some embodiments, a linking group conjugated to an RNAi agent may be a trialkyne linking group.

Examples of certain modified nucleotides and linking groups, are provided in Table 22.

TABLE 22
Structures Representing Various Modified Nucleotides and Linking Groups.

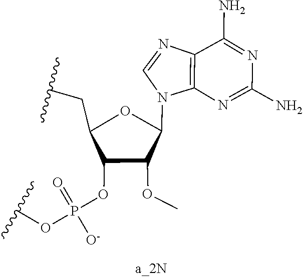

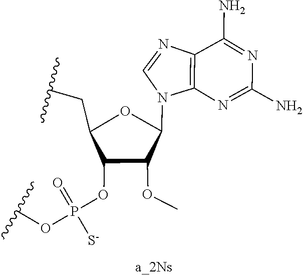

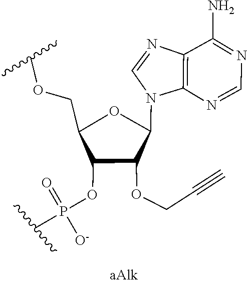

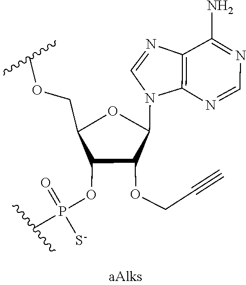

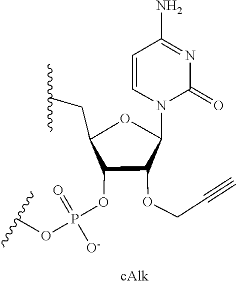

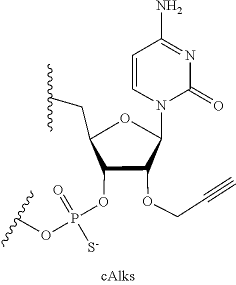

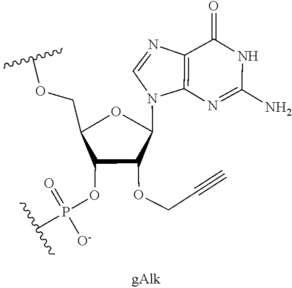

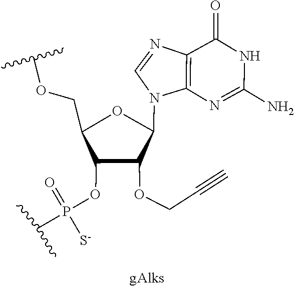

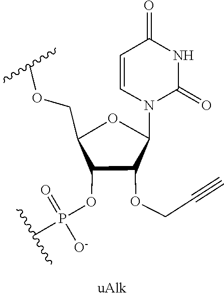

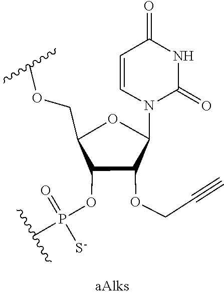

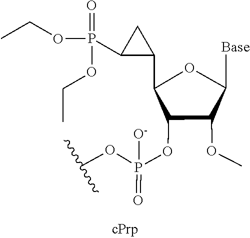

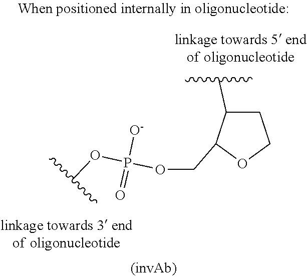 [SMILES: *OP(=O)([O-])OCC1OCCC1*]

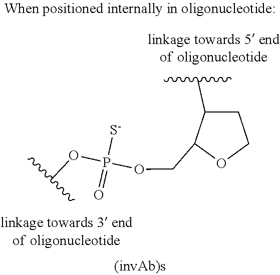 [SMILES: *OP(=O)([S-])OCC1OCCC1*]

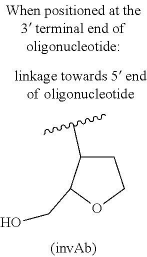 [SMILES: *C1CCOC1CO]

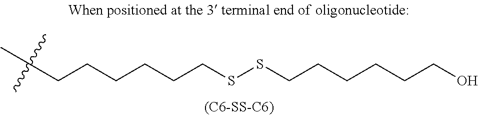 [SMILES: *CCCCCCSSCCCCCCO]

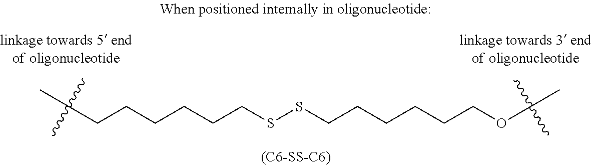 [SMILES: *CCCCCCSSCCCCCCO*]

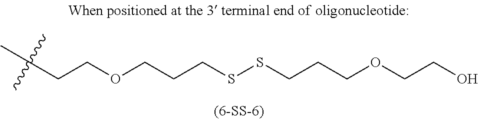 [SMILES: *CCOCCCSSCCCOCCO]

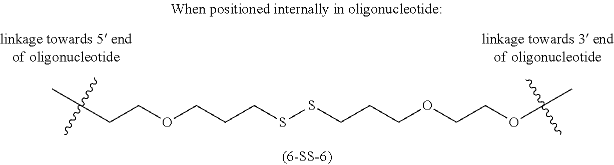 [SMILES: *CCOCCCSSCCCOCCO*]

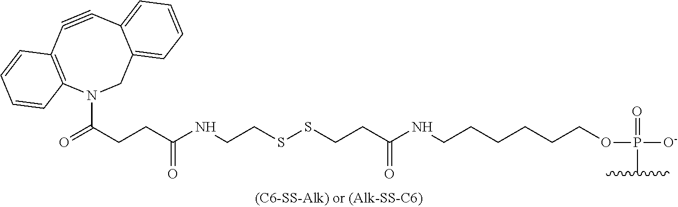 [SMILES: *P(=O)([O-])OCCCCCCNC(=O)CCSSCCNC(=O)CCC(=O)N1Cc2ccccc2C#Cc2ccccc21]

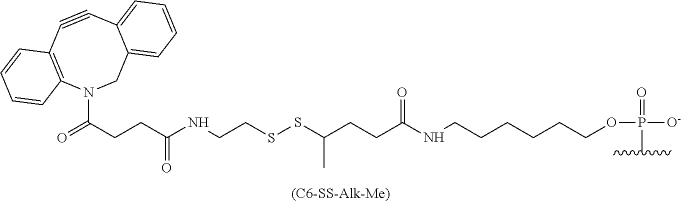 [SMILES: *P(=O)([O-])OCCCCCCNC(=O)CCC(C)SSCCNC(=O)CCC(=O)N1Cc2ccccc2C#Cc2ccccc21]

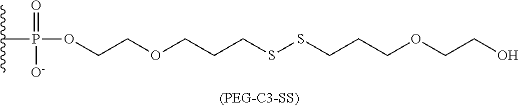 [SMILES: *P(=O)([O-])OCCOCCCSSCCCOCCO]

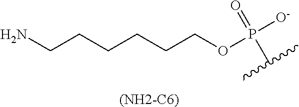 [SMILES: *P(=O)([O-])OCCCCCCN ; "(NH2-C6)"]

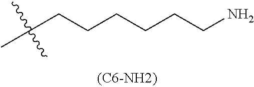 [SMILES: *CCCCCCN]

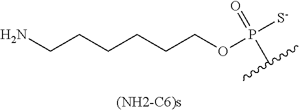 [SMILES: *P(=O)([S-])OCCCCCCN]

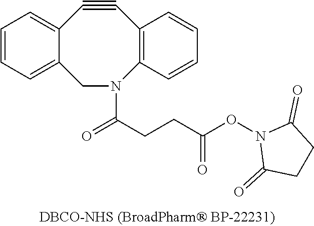 [SMILES: O=C(CCC(=O)N1Cc2ccccc2C#Cc2ccccc21)ON1C(=O)CCC1=O]

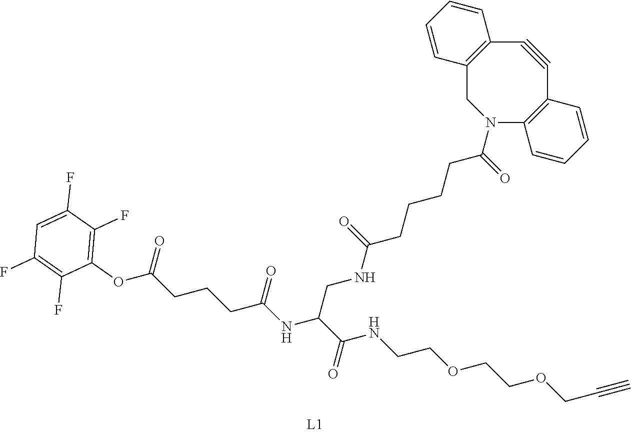 [SMILES: C#CCOCCOCCNC(=O)C(CNC(=O)CCCCC(=O)N1Cc2ccccc2C#Cc2ccccc21)NC(=O)CCCC(=O)Oc1c(F)c(F)cc(F)c1F]

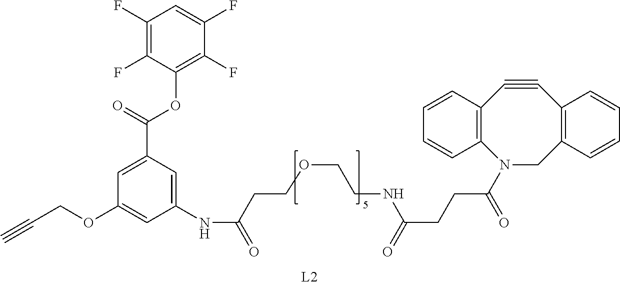

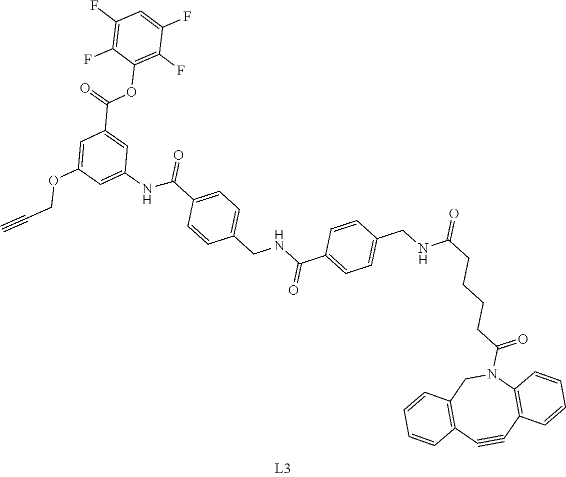 [SMILES: C#CCOc1cc(NC(=O)c2ccc(CNC(=O)c3ccc(CNC(=O)CCCCC(=O)N4Cc5ccccc5C#Cc5ccccc54)cc3)cc2)cc(C(=O)Oc2c(F)c(F)cc(F)c2F)c1]

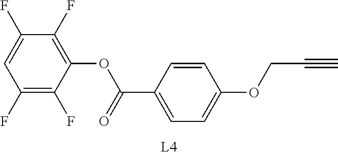 [SMILES: C#CCOc1ccc(C(=O)Oc2c(F)c(F)cc(F)c2F)cc1]

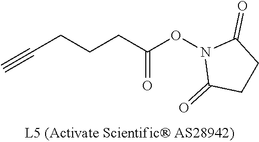

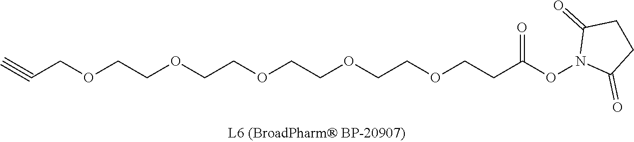 [SMILES: C#CCOCCOCCOCCOCCOCCC(=O)ON1C(=O)CCC1=O]

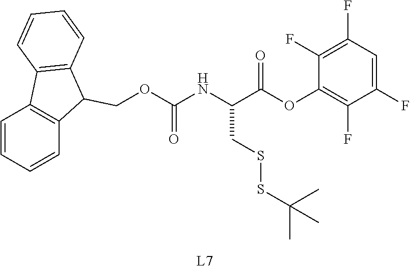 [SMILES: CC(C)(C)SSC[C@H](NC(=O)OCC1c2ccccc2-c2ccccc21)C(=O)Oc1c(F)c(F)cc(F)c1F]

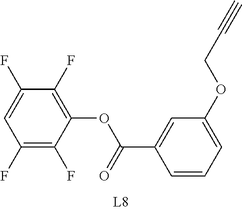 [SMILES: C#CCOc1cccc(C(=O)Oc2c(F)c(F)cc(F)c2F)c1]

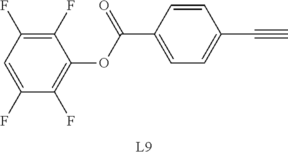 [SMILES: C#Cc1ccc(C(=O)Oc2c(F)c(F)cc(F)c2F)cc1]

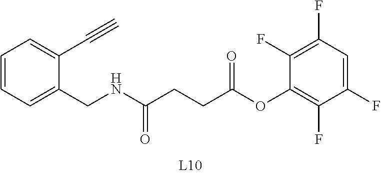 [SMILES: C#Cc1ccccc1CNC(=O)CCC(=O)Oc1c(F)c(F)cc(F)c1F]

Alternatively, other linking groups known in the art may be used.

In addition or alternatively to linking an RNAi agent to one or more targeting ligands, targeting groups, and/or PK/PD modulators, in some embodiments, a delivery agent may be used to deliver an RNAi agent to a cell or tissue. A delivery agent is a compound that can improve delivery of the RNAi agent to a cell or tissue, and can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/022309, WO 2011/104169, and WO 2012/083185, WO 2013/032829, WO 2013/158141, each of which is incorporated herein by reference), or other delivery systems available in the art.

Pharmaceutical Compositions

In some embodiments, the present disclosure provides pharmaceutical compositions that include, consist of, or consist essentially of, one or more compounds of Formula (I), (Ia), (Ib), (Ib1), (Ic), (Id), (II), (III), (IIIa), (IIIb), (IV), or (IVa).

As used herein, a “pharmaceutical composition” comprises a pharmacologically effective amount of an Active Pharmaceutical Ingredient (API), and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions described herein can contain other additional components commonly found in pharmaceutical compositions. In some embodiments, the additional component is a pharmaceutically-active material. Pharmaceutically-active materials include, but are not limited to: anti-pruritics, astringents, local anesthetics, or anti-inflammatory agents (e.g., antihistamine, diphenhydramine, etc.), small molecule drug, antibody, antibody fragment, aptamers, and/or vaccines.

The pharmaceutical compositions may also contain preserving agents, solubilizing agents, stabilizing agents, wetting agents, emulsifiers, sweeteners, colorants, odorants, salts for the variation of osmotic pressure, buffers, coating agents, or antioxidants. They may also contain other agent with a known therapeutic benefit.

The pharmaceutical compositions can be administered in a number of ways depending upon whether local or systemic treatment is desired and upon the area to be treated. Administration can be made by any way commonly known in the art, such as, but not limited to, topical (e.g., by a transdermal patch), pulmonary (e.g., by inhalation or insufflation of powders or aerosols, including by nebulizer, intratracheal, intranasal), epidermal, transdermal, oral or parenteral. Parenteral administration includes, but is not limited to, intravenous, intraarterial, subcutaneous, intraperitoneal or intramuscular injection or infusion; subdermal (e.g., via an implanted device), intracranial, intraparenchymal, intrathecal, and intraventricular, administration. In some embodiments, the pharmaceutical compositions described herein are administered by subcutaneous injection. The pharmaceutical compositions may be administered orally, for example in the form of tablets, coated tablets, dragées, hard or soft gelatin capsules, solutions, emulsions or suspensions. Administration can also be carried out rectally, for example using suppositories; locally or percutaneously, for example using ointments, creams, gels, or solutions; or parenterally, for example using injectable solutions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL (BASF, Parsippany, N.J.) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of any of the ligands described herein that can be in microcrystalline form, for example, in the form of an aqueous microcrystalline suspension. Liposomal formulations or biodegradable polymer systems can also be used to present any of the ligands described herein for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). As used herein, “pharmacologically effective amount,” “therapeutically effective amount,” or simply “effective amount” refers to that amount of an the pharmaceutically active agent to produce a pharmacological, therapeutic or preventive result.

Medicaments containing compounds of Formula (I), (Ia), (Ib), (Ib1), (Ic), (Id), (II), (III), (IIIa), (IIIb), (IV), or (IVa) are also an object of the present invention, as are processes for the manufacture of such medicaments, which processes comprise bringing one or more compound of Formula (I), (Ia), (Ib), (Ib1), (Ic), (Id), (II), (III), (IIIa), (IIIb), (IV), or (IVa), and, if desired, one or more other substances with a known therapeutic benefit, into a pharmaceutically acceptable form.

The described compounds of Formula (I), (Ia), (Ib), (Ib1), (Ic), (Id), (II), (III), (IIIa), (IIIb), (IV), or (IVa) and pharmaceutical compositions comprising compounds of Formula (I), (Ia), (Ib), (Ib1), (Ic), (Id), (II), (III), (IIIa), (IIIb), (IV), or (IVa) disclosed herein may be packaged or included in a kit, container, pack, or dispenser. The compounds of Formula (I), (Ia), (Ib), (Ib1), (Ic), (Id), (II), (III), (IIIa), (IIIb), (IV), or (IVa) and pharmaceutical compositions comprising the compounds of Formula (I), (Ia), (Ib), (Ib1), (Ic), (Id), (II), (III), (IIIa), (IIIb), (IV), or (IVa) may be packaged in pre-filled syringes or vials.

Methods of Treatment and Inhibition of Expression

The compounds of Formula (I), (Ia), (Ib), (Ib1), (Ic), (Id), (II), (III), (IIIa), (IIIb), (IV), or (IVa) disclosed herein can be used to treat a subject (e.g., a human or other mammal) having a disease or disorder that would benefit from administration of such compounds. In some embodiments, the compounds of Formula (I), (Ia), (Ib), (Ib1), (Ic), (Id), (II), (III), (IIIa), (IIIb), (IV), or (IVa) disclosed herein can be used to treat a subject (e.g., a human) that would benefit from reduction and/or inhibition in expression of a target mRNA and/or protein levels, for example, a subject that has been diagnosed with or is suffering from symptoms related to muscular dystrophy.

In some embodiments, the subject is administered a therapeutically effective amount of one or more compounds of Formula (I), (Ia), (Ib), (Ib1), (Ic), (Id), (II), (III), (IIIa), (IIIb), (IV), or (IVa) disclosed herein. Treatment of a subject can include therapeutic and/or prophylactic treatment. The subject is administered a therapeutically effective amount of one or more compounds of Formula (I), (Ia), (Ib), (Ib1), (Ic), (Id), (II), (III), (IIIa), (IIIb), (IV), or (IVa) described herein. The subject can be a human, patient, or human patient. The subject may be an adult, adolescent, child, or infant. Administration of a pharmaceutical composition described herein can be to a human being or animal.

The compounds of Formula (I), (Ia), (Ib), (Ib1), (Ic), (Id), (II), (III), (IIIa), (IIIb), (IV), or (IVa) described herein can be used to treat at least one symptom in a subject having a disease or disorder related to a target gene, or having a disease or disorder that is mediated at least in part by the expression of the target gene. In some embodiments, the compounds of Formula (I), (Ia), (Ib), (Ib1), (Ic), (Id), (II), (III), (IIIa), (IIIb), (IV), or (IVa) are used to treat or manage a clinical presentation of a subject with a disease or disorder that would benefit from or be mediated at least in party by a reduction in mRNA of a target gene. The subject is administered a therapeutically effective amount of one or more of the compounds of Formula (I), (Ia), (Ib), (Ib1), (Ic), (Id), (II), (III), (IIIa), (IIIb), (IV), or (IVa) or compositions described herein. In some embodiments, the methods disclosed herein comprise administering a composition comprising a compound of Formula (I), (Ia), (Ib), (Ib1), (Ic), (Id), (II), (III), (IIIa), (IIIb), (IV), or (IVa) described herein to a subject to be treated. In some embodiments, the subject is administered a prophylactically effective amount of any one or more of the described compounds of Formula (I), (Ia), (Ib), (Ib1), (Ic), (Id), (II), (III), (IIIa), (IIIb), (IV), or (IVa), thereby treating the subject by preventing or inhibiting the at least one symptom.

In certain embodiments, the present disclosure provides methods for treatment of diseases, disorders, conditions, or pathological states mediated at least in part by target gene expression, in a patient in need thereof, wherein the methods include administering to the patient any of the compounds of Formula (I), (Ia), (Ib), (Ib1), (Ic), (Id), (II), (III), (IIIa), (IIIb), (IV), or (IVa) described herein.

In some embodiments, the gene expression level and/or mRNA level of a target gene in a subject to whom a compound of Formula (I), (Ia), (Ib), (Ib1), (Ic), (Id), (II), (III), (IIIa), (IIIb), (IV), or (IVa) described herein is administered is reduced by at least about 30%, 35%, 40%, 45%, 50%, 55%, 60%, 65%, 70%, 75%, 80%, 85%, 95%, 96%, 97%, 98%, 99%, or greater than 99% relative to the subject prior to being administered the compound or to a subject not receiving the compound. The gene expression level and/or mRNA level in the subject may be reduced in a cell, group of cells, and/or tissue of the subject.

In some embodiments, the target protein level in a subject to whom a compound of Formula (I), (Ia), (Ib), (Ib1), (Ic), (Id), (II), (III), (IIIa), (IIIb), (IV), or (IVa) described herein has been administered is reduced by at least about 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the compound or to a subject not receiving the compound. The protein level in the subject may be reduced in a cell, group of cells, tissue, blood, and/or other fluid of the subject.

A reduction in target mRNA levels and/or target protein levels can be assessed by any methods known in the art. As used herein, a reduction or decrease in target mRNA level and/or protein level are collectively referred to herein as a reduction or decrease in target gene and/or protein levels or inhibiting or reducing the expression of a target gene.

In some embodiments, compounds of Formula (I), (Ia), (Ib), (Ib1), (Ic), (Id), (II), (III), (IIIa), (IIIb), (IV), or (IVa) described herein may be used in the preparation of a pharmaceutical composition for use in the treatment of a disease, disorder, or symptom that is mediated at least in part by target gene expression. In some embodiments, the disease, disorder, or symptom that is mediated at least in part by target gene expression is a muscular dystrophy.

In some embodiments, methods of treating a subject are dependent on the body weight of the subject. In some embodiments, compounds of Formula (I), (Ia), (Ib), (Ib1), (Ic), (Id), (II), (III), (IIIa), (IIIb), (IV), or (IVa) may be administered at a dose of about 0.05 mg/kg to about 40.0 mg/kg of body weight of the subject. In other embodiments compounds of Formula (I), (Ia), (Ib), (Ib1), (Ic), (Id), (II), (III), (IIIa), (IIIb), (IV), or (IVa) may be administered at a dose of about 5 mg/kg to about 20 mg/kg of body weight of the subject.

In some embodiments, compounds of Formula (I), (Ia), (Ib), (Ib1), (Ic), (Id), (II), (III), (IIIa), (IIIb), (IV), or (IVa) may be administered in a split dose, meaning that two doses are given to a subject in a short (for example, less than 24 hour) time period. In some embodiments, about half of the desired daily amount is administered in an initial administration, and the remaining about half of the desired daily amount is administered approximately four hours after the initial administration.

In some embodiments, compounds of Formula (I), (Ia), (Ib), (Ib1), (Ic), (Id), (II), (III), (IIIa), (IIIb), (IV), or (IVa) described herein may be administered once a week (i.e., weekly). In other embodiments, compounds of Formula (I), (Ia), (Ib), (Ib1), (Ic), (Id), (II), (III), (IIIa), (IIIb), (IV), or (IVa) described herein may be administered biweekly (once every other week).

In some embodiments, compounds of Formula (I), (Ia), (Ib), (Ib1), (Ic), (Id), (II), (III), (IIIa), (IIIb), (IV), or (IVa) described herein or compositions containing such compounds may be used for the treatment of a disease, disorder, or symptom that is mediated at least in part by target gene expression. In some embodiments, the disease, disorder or symptom that is mediated at least in part by target gene expression is muscular dystrophy.

Another aspect of the invention provides for a method of reducing a target gene expression in vivo, the method comprising introducing to a cell a compound of Formula (I), (Ia), (Ib), (Ib1), (Ic), (Id), (II), (III), (IIIa), (IIIb), (IV), or (IVa) described herein, wherein the compound comprises an RNAi agent at least substantially complementary to the target gene. In some embodiments, the cell is a skeletal muscle cell. In some embodiments, the cell is within a subject. In some embodiments, the subject has been diagnosed with a disease or disorder that is treated, prevented or ameliorated by reducing expression of the target gene. In some embodiments, the disease or disorder is a muscular dystrophy selected from the group consisting of Duchenne muscular dystrophy, myotonic muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy.

Another aspect of the invention provides for the use of any one of the lipid PK/PD modulators conjugated to an oligonucleotide-based agent described herein for the treatment, prevention, or amelioration of a disease or disorder. In some embodiments, the disease or disorder is a muscular dystrophy selected from the group consisting of Duchenne muscular dystrophy, myotonic muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy.

Cells, Tissues, and Non-Human Organisms

Cells, tissues, and non-human organisms that include at least one of the compounds of Formula (I), (Ia), (Ib), (Ib1), (Ic), (Id), (II), (III), (IIIa), (IIIb), (IV), or (IVa) described herein is contemplated. The cell, tissue, or non-human organism is made by delivering the compound of Formula (I), (Ia), (Ib), (Ib1), (Ic), (Id), (II), (III), (IIIa), (IIIb), (IV), or (IVa) to the cell, tissue, or non-human organism by any means available in the art. In some embodiments, the cell is a mammalian cell, including, but not limited to, a human cell. In some embodiments the cell is a skeletal muscle cell.

The above provided embodiments and items are now illustrated with the following, non-limiting examples.

### EXAMPLES

The following examples are not limiting and are intended to illustrate certain embodiments disclosed herein.

Unless expressly stated otherwise, numerals used to refer to compounds of a given example are only made with reference to that particular example and not any other examples disclosed herein. For example, compound 1 of “Synthesis of LP1-p” in Example 4 is different from, and does not refer to, compound 1 of “Synthesis of LP-5p” in Example 4. Similarly, it will be appreciated that a particular compound disclosed herein may be identified by different numerals in different examples. For example, compound 12 of “Synthesis of LP223-p” in Example 4 is the same as compound 3 of “Synthesis of LP224-p” in Example 4. Compounds that are disclosed in various tables throughout the detailed description (i.e., LPXXa, LPXXb, and LPXX-p, wherein XX is a number) are referred to consistently throughout the examples herein.

TABLE 23
Some common abbreviations used in the examples.

| Name | Abbreviation(s) |
|---|---|
| Triethylamine | TEA, NEt₃ |
| Dichloromethane | DCM, CH₂Cl₂ |
| Ethyl acetate | EA, EtOAc |
| Hexanes | Hex |
| Methanol | MeOH |
| Acetonitrile | ACN, MeCN |
| Trifluoroacetic acid | TFA |
| Acetic acid | AcOH |
| Fluorenylmethyloxycarbonyl | FMOC |
| tert-Butyloxycarbonyl | BOC |
| Dimethylformamide | DMF |
| Toluene | PhMe, Tol. |
| 1-Ethyl-3-(3- | EDC |
| dimethylaminopropyl)carbodiimide | |
| Triisopropylsilane | TIS, TIPS |
| 2-(1H-Benzotriazole-1-yl)-1,1,3,3- | TBTU |
| tetramethylaminium tetrafluoroborate | |
| N,N-Di-isopropylethylamine | DIPEA, DIEA, |
| | i-Pr₂NEt |
| 2-(1H-benzotriazol-1-yl)-1,1,3,3- | HBTU |
| tetramethyluronium hexafluorophosphate | |
| 1-Cyano-2-ethoxy-2- | COMU |
| oxoethylidenaminooxy)dimethylamino- | |
| morpholino-carbenium hexafluorophosphate | |
| 1-[Bis(dimethylamino)methylene]-1H-1,2,3- | HATU |
| triazolo[4,5-b]pyridinium 3-oxide | |
| hexafluorophosphate,\ | |
| N-Hydroxysuccinimide | NHS |
| Dibenzocyclooctyne | DBCO |
| Tri(2-furyl)phosphine | TFP |
| Tetrahydrofuran | THF |
| Hydrochloric acid | HCl |
| Methyl Iodide | MeI, CH₃I |
| 4-Dimethylaminopyridine | DMAP |
| meta-Chloroperoxybenzoic acid | mCPBA |
| Carbon disulfide | CS₂ |
| Sodium Hydroxide | NaOH |
| Anhydrous | Anhyd |
| Aqueous | Aq |
| Equivalent/Equivalents | Eq, Equiv |
| Saturated | Sat’d, Sat |

It will be appreciated that, unless expressly stated otherwise, use of the term “EDC” in the examples herein refers to the EDC hydrochloride salt which is commercially available.

### Example 1. Syntheses of RNAi Agents and Compositions

The following describes the general procedures for the syntheses of certain RNAi agents, and conjugates thereof, that are illustrated in the non-limiting Examples set forth herein.

Synthesis of RNAi Agents. RNAi agents can be synthesized using methods generally known in the art. For the synthesis of the RNAi agents illustrated in the Examples set forth herein, the sense and antisense strands of the RNAi agents were synthesized according to solid phase phosphoramidite technology used in oligonucleotide synthesis. Depending on the scale, a MerMade96E® (Bioautomation), a MerMade12® (Bioautomation), or an Oligopilot 100 (GE Healthcare) was used. Syntheses were performed on a solid support made of controlled pore glass (CPG, 500 Å or 600 Å, obtained from Prime Synthesis, Aston, Pa., USA) or polystyrene (obtained from Kinovate, Oceanside, Calif., USA). All RNA and 2′-modified RNA phosphoramidites were purchased from Thermo Fisher Scientific (Milwaukee, Wis., USA), ChemGenes (Wilmington, Mass., USA), or Hongene Biotech (Morrisville, N.C., USA). Specifically, the following 2′-O-methyl phosphoramidites that were used include the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites and 2′-O-propargyl phosphoramidites carried the same protecting groups as the 2′-O-methyl phosphoramidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes. The following UNA phosphoramidites that were used included the following: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-di-isopropyl)]-phosphoramidite. In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile or a 200 mM solution of xanthane hydride (TCI America, Portland, Oreg., USA) in pyridine was employed.

TFA aminolink phosphoramidites were also commercially purchased (ThermoFisher) to introduce the (NH2-C6) reactive group linkers. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ O-Me), and 60 sec (2′ F). Trialkyne-containing phosphoramidites were synthesized to introduce the respective (TriAlk #) linkers. When used in connection with the RNAi agents presented in certain Examples herein, trialkyne-containing phosphoramidites were dissolved in anhydrous dichloromethane or anhydrous acetonitrile (50 mM), while all other amidites were dissolved in anhydrous acetonitrile (50 mM), and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ O-Me), and 60 sec (2′ F).

For some RNAi agents, a linker, such as a C6-SS-C6 or a 6-SS-6 group, was introduced at the 3′ terminal end of the sense strand. Pre-loaded resin was commercially acquired with the respective linker. Alternatively, for some sense strands, a dT resin was used and the respectively linker was then added via standard phosphoramidite synthesis.

Cleavage and deprotection of support bound oligoner. After finalization of the solid phase synthesis, the dried solid support was treated with a 1:1 volume solution of 40 weight (wt.) % methylamine in water and 28% to 31% ammonium hydroxide solution (Aldrich) for 1.5 hours at 30° C. The solution was evaporated and the solid residue was reconstituted in water (see below).

Purification. Crude oligomers were purified by anionic exchange HPLC using a TSKgel® SuperQ-5PW 13 μm column (available from Tosoh Biosciences) and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex® G25 fine (available from Sigman Aldrich) with a running buffer of 100 mM ammonium bicarbonate, pH 6.7 and 20% Acetonitrile or filtered water.

Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor and the dilution factor to determine the duplex concentration. The conversion factor used was either 0.037 mg/(mL-cm) or was calculated from an experimentally determined extinction coefficient.

Post-Synthetic Conjugation of Trialkyne scaffold. Either prior to or after annealing, the 5′ or 3′ amine functionalized sense strand of an RNAi agent can be conjugated to a trialkyne scaffold. The following describes the conjugation of trialkyne scaffold to the annealed duplex: Amine functionalized duplex was dissolved in 90% DMSO/10% H₂O, at approximately 50-70 mg/mL. 40 eq triethylamine (TEA) was added, followed by 3 eq trialkyne-PNP. Once complete, the conjugate was precipitated twice in a solvent system of 1×phosphate buffered saline/acetonitrile (1:14 ratio), and dried.

### Example 2. Synthesis of Linking Groups

### Synthesis of L₄

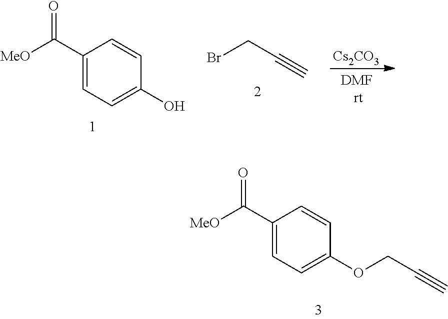

To a solution of compound 1 (3.00 g) in DMF was added Cs₂CO₃ (7.71 g) at room temperature. Compound 2 (1.85 mL) was then added slowly. The resulting reaction mixture was stirred overnight under N₂(g). Approximately full conversion to desired product by LC-MS was then confirmed. The reaction mixture was quenched with NaHCO₃ (10 mL). The product was extracted with EtOAc (5×10 mL) and then washed with water (3×8 mL) and brine (8 mL). The combined organic phases were dried over Na₂SO₄, filtered, and concentrated. The residue was purified by CombiFlash® using silica gel as the stationary phase with a gradient of hexanes to EtOAc (0-30%), in which the product eluted at 14% B. Compound 3 was concentrated under vacuum to provide a white solid. LC-MS: calculated [M+H]+ 191.06 m/z, observed 191.23 m/z.

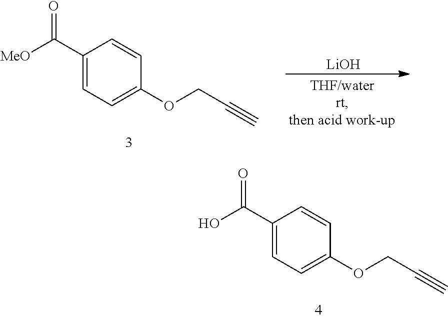

To a solution of compound 3 (2.87 g) in 1:1 THF/water was added LiOH (1.08 g) at room temperature under normal atmosphere. The reaction mixture was stirred until full conversion of compound 3 was observed by LC-MS. Residual starting material was extracted via EtOAc, and then aqueous phase was acidified with 6 N HCl to a pH of approximately 3. Compound 4 crashed out as a white solid and was filtered over vacuum and washed with water. Due to its wet/sticky nature, solvent was required to transfer the solid to a round bottom flask; material was transferred via MeOH and DCM. Due to poor solvation in either solvent and the combination, the material could not to be dried over Na₂SO₄. Compound 4 was concentrated under vacuum to provide a white, fluffy crystalline solid and was used directly without further purification. LC-MS: calculated [M+H]+ 177.05 m/z, observed 177.19 m/z.

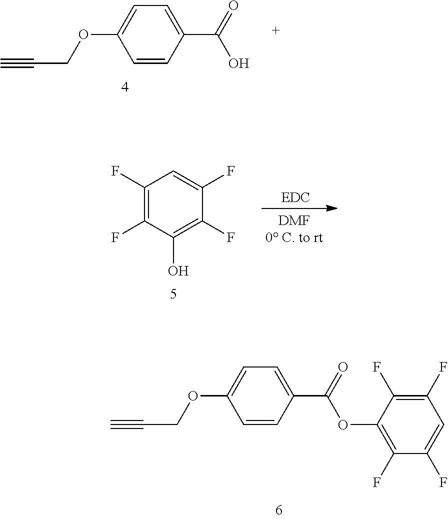 [SMILES: C#CCOc1ccc(C(=O)O)cc1 ; Oc1c(F)c(F)cc(F)c1F ; C#CCOc1ccc(C(=O)Oc2c(F)c(F)cc(F)c2F)cc1]

To a solution of compounds 4 (1.00 g) and 5 (1.04 g) in DMF (10.0 mL) under N₂(g) was added EDC (1.20 g) at room temperature. The reaction mixture was allowed to stir until full conversion was observed by LC-MS. Due to an inability to successfully observe the product after overnight stirring, the reaction mixture was quenched with NaHCO₃. The resulting precipitate was confirmed to contain starting materials via LC-MS and was filtered over vacuum, attempted to be re-suspended in MeOH/DCM, and then concentrated under vacuum. The mixture was then re-solvated in DMF, dried over Na₂SO₄, filtered over vacuum, and rinsed with DMF. EDC was re-added to the filtrate (i.e., compounds 4 and 5) in DMF, and the resultant mixture was allowed to stir overnight at room temperature. The reaction mixture was directly concentrated and azeotroped with MeOH and PhMe for isolation. The residue was purified by CombiFlash® using silica gel as the stationary phase and was eluted with 0-20% MeOH in DCM. L₄ eluted at 0% B to provide a white solid. LC-MS: calculated [M+H]+ 325.04 m/z, observed 325.35 m/z.

### Example 3. Synthesis of Targeting Ligands

### Synthesis of αvβ6 Peptide 1

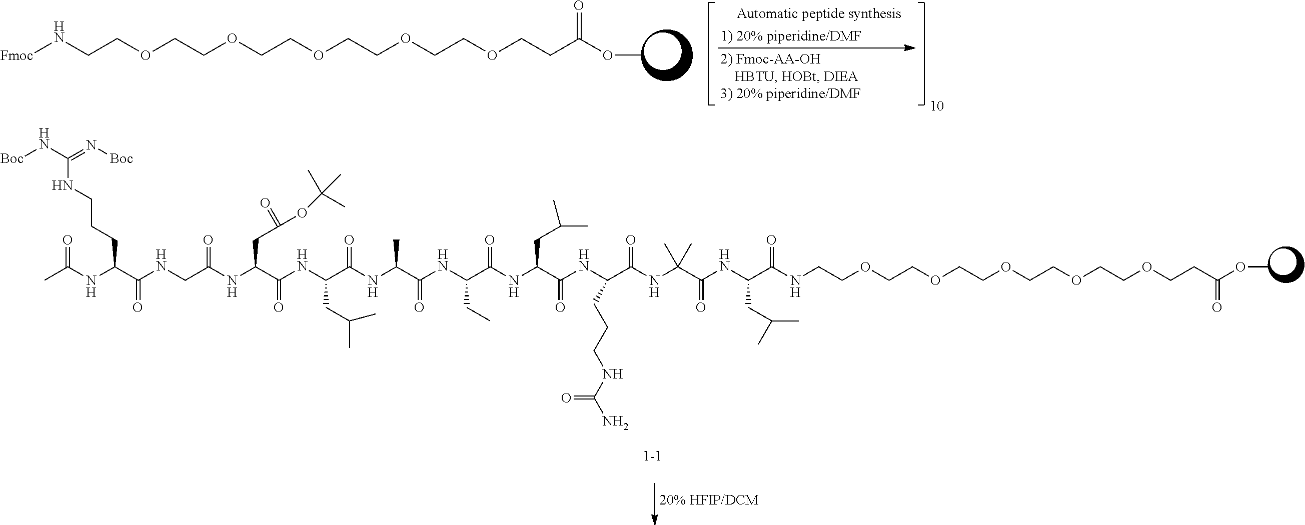

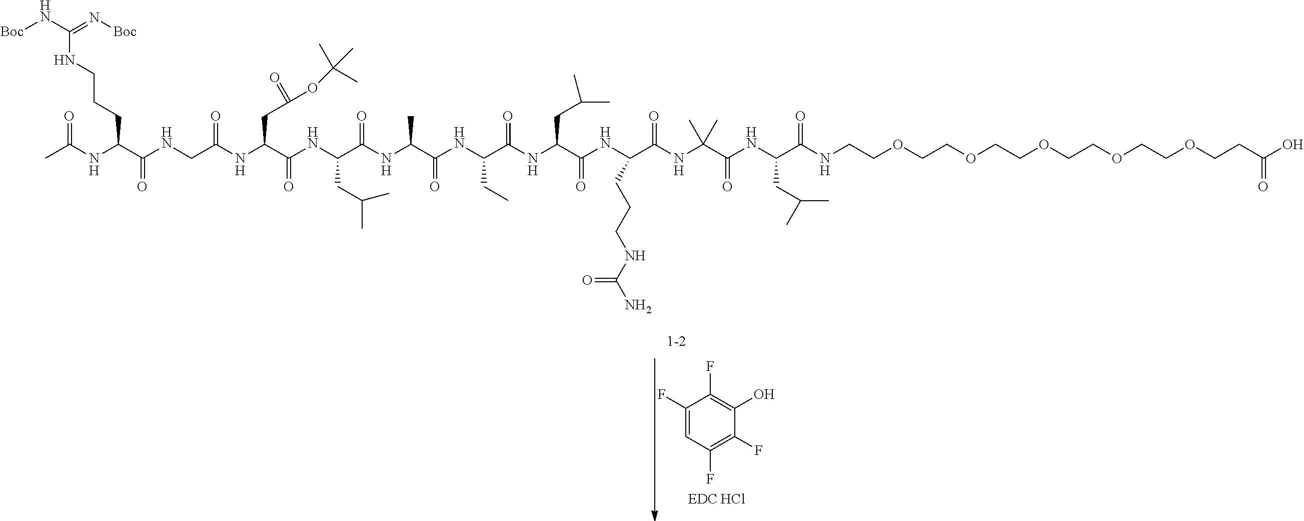 [SMILES: CC[C@H](NC(=O)[C@H](C)NC(=O)[C@H](CC(C)C)NC(=O)[C@H](CC(=O)OC(C)(C)C)NC(=O)CNC(=O)[C@H](CCCN/C(=N\C(=O)OC(C)(C)C)NC(=O)OC(C)(C)C)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)O ; Oc1c(F)c(F)cc(F)c1F]

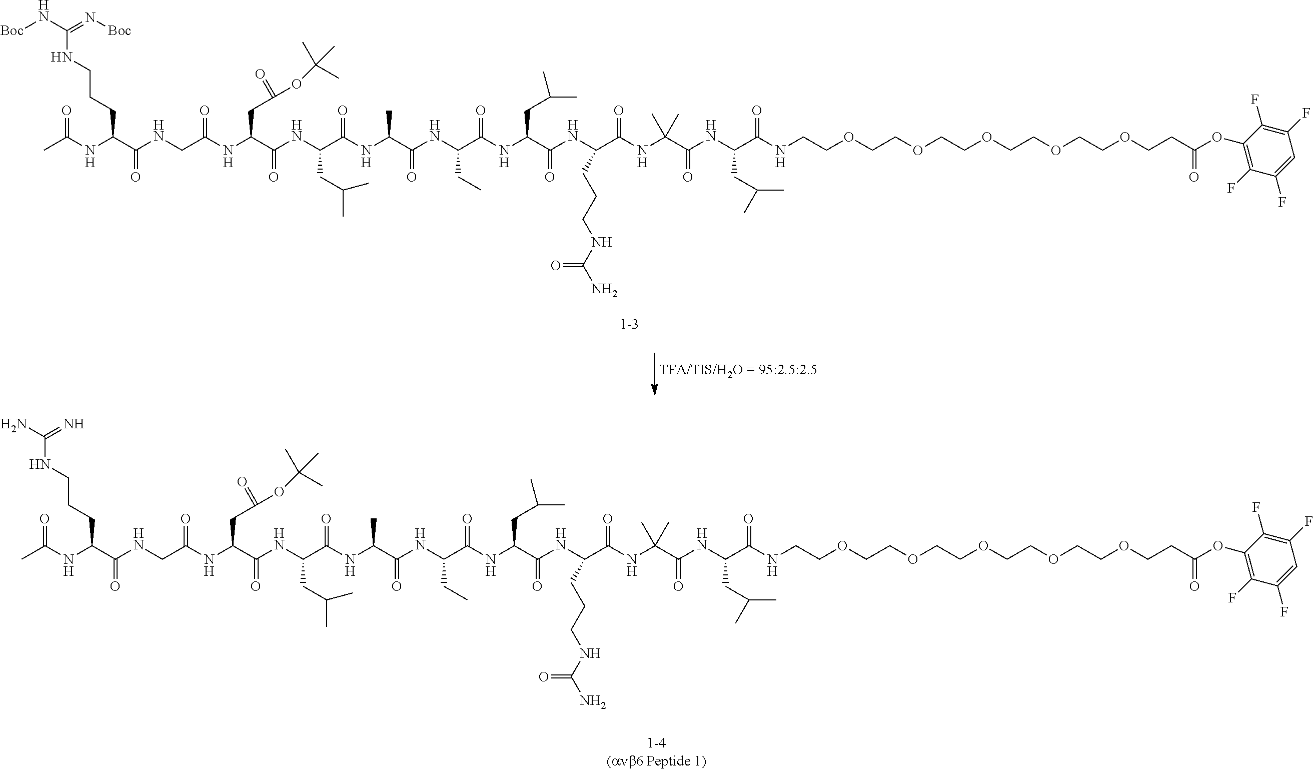 [SMILES: CC[C@H](NC(=O)[C@H](C)NC(=O)[C@H](CC(C)C)NC(=O)[C@H](CC(=O)OC(C)(C)C)NC(=O)CNC(=O)[C@H](CCCN/C(=N\C(=O)OC(C)(C)C)NC(=O)OC(C)(C)C)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)Oc1c(F)c(F)cc(F)c1F ; CC[C@H](NC(=O)[C@H](C)NC(=O)[C@H](CC(C)C)NC(=O)[C@H](CC(=O)OC(C)(C)C)NC(=O)CNC(=O)[C@H](CCCNC(=N)N)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)Oc1c(F)c(F)cc(F)c1F]

αvβ6 Peptide 1 was prepared by modification of Arg-Gly-Asp(tBu)-Leu-Ala-Abu-Leu-Cit-Aib-Leu-Peg₅-CO₂-2-Cl-Trt resin 1-1 that was obtained using general Fmoc peptide chemistry on a CS Bio peptide synthesizer utilizing Fmoc-Peg₅-CO₂H preloaded 2-Cl-Trt resin on (0.79 mmol/g) at 4.1 mmol scale as described above. Following cleavage from the resin, the peptide 1-2 was converted into the tetrafluorophenyl ester 1-3, and the crude product was used in the next step without purification.

Final deprotection was done by treatment of crude peptide 1-3 with the deprotection cocktail TFA/TIS/H₂O=90:5:5 (80 mL) for 1.5 h. The reaction mixture was added dropwise to methyl tert-butyl ether (700 mL), and the resulting precipitate was collected by centrifugation. The pellets were washed with additional methyl tert-butyl ether (500 mL). The residue was purified by reverse phase (RP)-HPLC (Phenomenex Gemini C18 250×50 mm, 10 micron, 60 mL/min, 30-45% ACN gradient in water containing 0.1% TFA, approximately 1 gram of crude per run), affording 4.25 g of pure peptide 1-4 (αvβ6 Peptide 1).

### Synthesis of αvβ6 Peptide 6

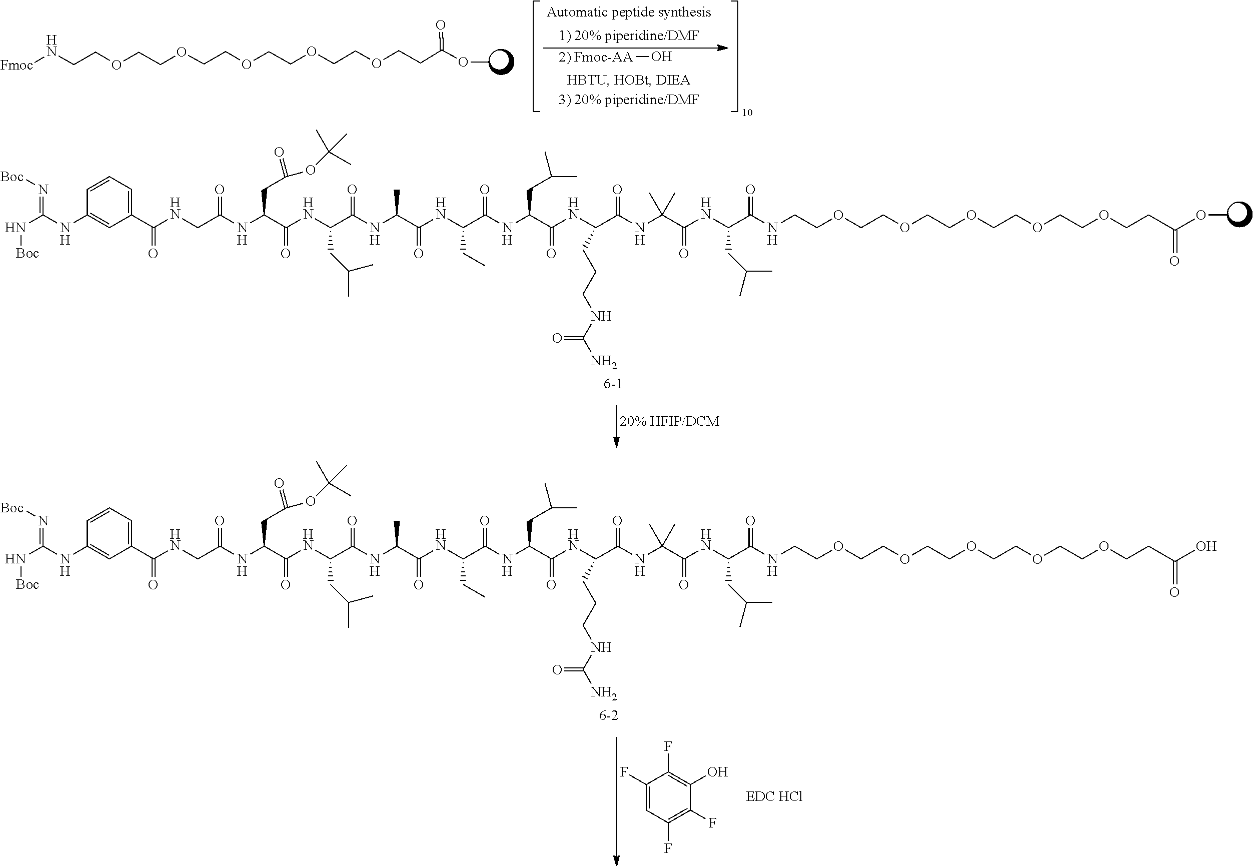

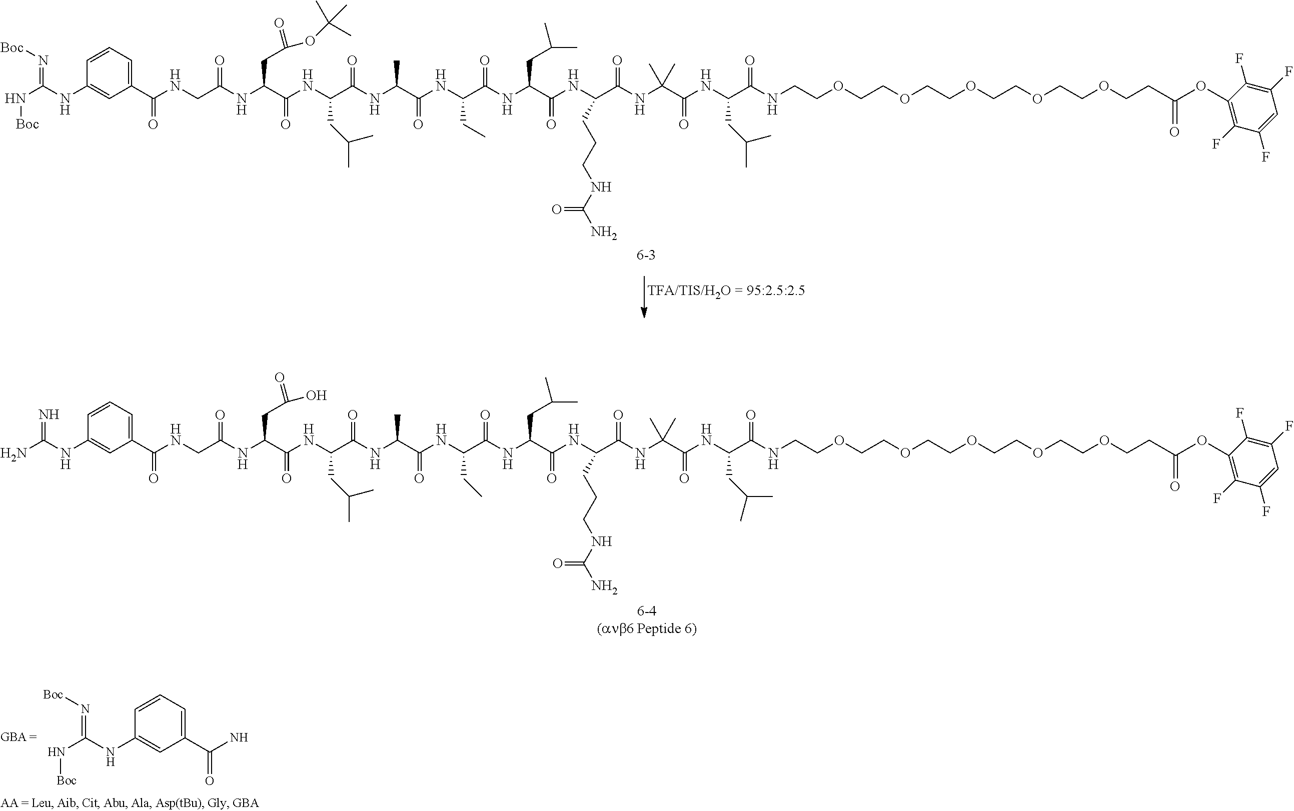

αvβ6 Peptide 6 was prepared by modification of GBA-Gly-Asp(tBu)-Leu-Ala-Abu-Leu-Cit-Aib-Leu-Peg₅-CO₂-2-Cl-Trt resin 6-1 that was obtained using general Fmoc peptide chemistry on a Symphony peptide synthesizer utilizing Fmoc-Peg₅-CO₂H preloaded 2-Cl-Trt resin on (0.85 mmol/g) at 0.2 mmol scale as described above. Following cleavage from the resin, the peptide 6-2 was converted into the tetrafluorophenyl ester 6-3, and purified on Combiflash® using the system 20% MeOH in DCM, gradient 15-100%, 25 minutes, to obtain 160 mg of pure peptide 6-3. Final deprotection was done by treatment of crude peptide 6-3 with the deprotection cocktail TFA/TIS/H₂O=90:5:5 (80 mL) for 1.5 h. The reaction mixture was added dropwise to methyl tert-butyl ether (700 mL), and the resulting precipitate was collected by centrifugation. The pellets were washed with additional methyl tert-butyl ether (500 mL). The residue was purified by HPLC purification using conditions: ACN (TFA 0.1%) in H₂O (TFA 0.1%) 27-57%, 25 minutes, Yield 94 mg. Calculated MW 1527.76, 1/2M=763.88. Found: MS (ES, pos): 1529.48 [M+1]⁺; 765.39 [M+2]²⁺.

### Synthesis of αvβ6 compound 45, (S)-3-(4-(4-((14-azido-3,6,9,12-tetraoxatetradecyl)oxy)naphthalen-1-yl)phenyl)-3-(2-(5-((4-methylpyridin-2-yl)amino)pentanamido)acetamido)propanoic acid

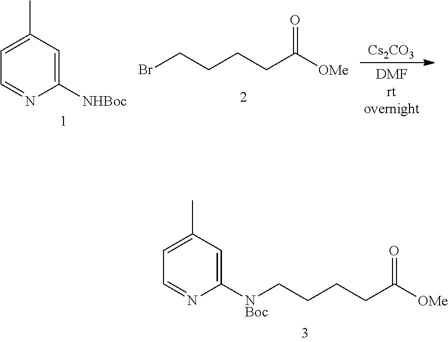

To a solution of compound 1 (0.50 g) in DMF under N₂(g) at room temperature was added Cs₂CO₃ (0.94 g). Compound 2 (0.49 g) was then added slowly dropwise. The reaction mixture was stirred overnight. Approximately 50% conversion to desired product was then confirmed by LC-MS. The reaction mixture was quenched with NaHCO₃ (10 mL). The product was extracted with EtOAc (3×15 mL) and then washed with water (3×10 mL) and brine (10 mL). The combined organic phases were dried over Na₂SO₄, filtered, and concentrated. The residue was purified by CombiFlash® using silica gel as the stationary phase with a gradient of 0-70% EtOAc in hexanes, in which the product eluted at 16% B. Compound 3 was concentrated under vacuum to provide a clear oil (0.35 g, 45.0% yield). LC-MS: calculated [M+H]+ 323.19 m/z, observed 328.38 m/z.

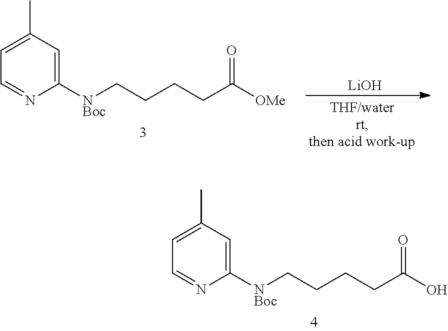 [SMILES: COC(=O)CCCCN(C(=O)OC(C)(C)C)c1cc(C)ccn1 ; "LiOH" ; Cc1ccnc(N(CCCCC(=O)O)C(=O)OC(C)(C)C)c1]

To a solution of compound 3 (0.35 g) in 1:1 THF/water was added LiOH (0.078 g) at room temperature under normal atmosphere. The reaction mixture was stirred at room temperature until full conversion was observed by LC-MS. After 1 hour, the reaction mixture was acidified with 6 N HCl to a pH of approximately 3. The product was extracted with EtOAc (3×15 mL). The combined organic phases were dried over Na₂SO₄, filtered, and concentrated to provide compound 4 as a clear, colorless oil (0.32 g, 94.9% yield). No isolation was necessary. LC-MS: calculated [M+H]+ 309.17 m/z, observed 309.24 m/z.

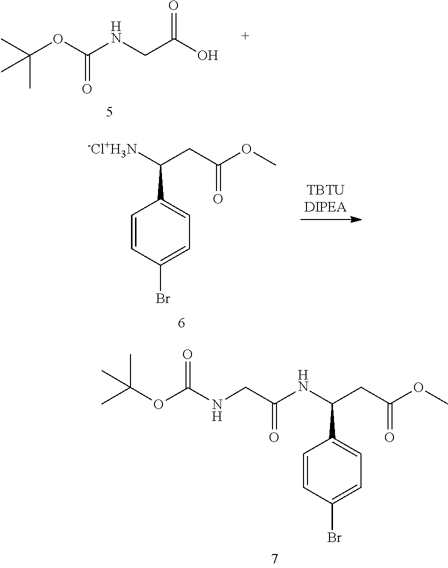

To a solution of compound 5 (1300 mg, 7.42 mmol, 1.0 equiv.), compound 6 (2295 mg, 7.792 mmol, 1.05 equiv.), and diisopropylethylamine (3.878 mL, 22.262 mmol, 3.0 equiv.) in anhydrous DMF (10 mL) was added TBTU (2859 mg, 8.905 mmol, 1.2 equiv.) at room temperature. The reaction mixture was kept at room temperature for 2 hours. The reaction mixture was quenched with saturated NaHCO₃ aqueous solution (5 mL) and the aqueous phase was extracted with ethyl acetate (3×5 mL). The combined organic phases were dried over anhydrous Na₂SO₄ and concentrated. The product was purified by CombiFlash® eluting with 2-4% MeOH in DCM. LC-MS: calculated [M+H]+ 415.08, found 415.29. Yield: 0.19 g, 6.04%.

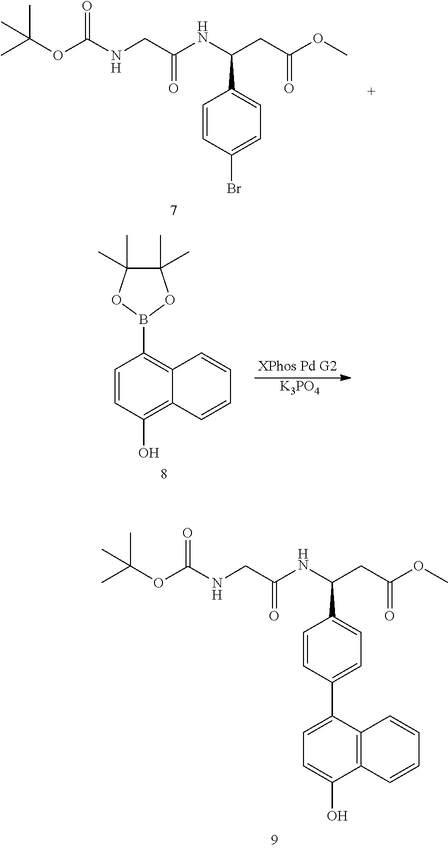

Compound 7 (3.20 g, 7.705 mmol, 1.0 equiv.), compound 8 (3.12 g, 11.558 mmol, 1.5 equiv.), XPhos Pd G2 (121 mg, 0.154 mmol, 0.02 equiv.), and K3PO4 (3.27 g, 15.411 mmol, 2.0 equiv.) were mixed in a round-bottom flask. The flask was sealed with a screw-cap septum, and then evacuated and backfilled with nitrogen (this process was repeated a total of 3 times). Then, THF (20 mL) and water (4 mL) were added via syringe. The reaction mixture was bubbled with nitrogen for 10 min and kept at 40° C. for 3 hours. The reaction mixture was quenched with saturated NaHCO₃ aqueous solution (20 mL), and the aqueous phase was extracted with ethyl acetate (3×20 mL). The combined organic phases were dried over Na₂SO₄, and concentrated. Compound 9 was purified by CombiFlash®, and was eluted with 2-4% MeOH in DCM.

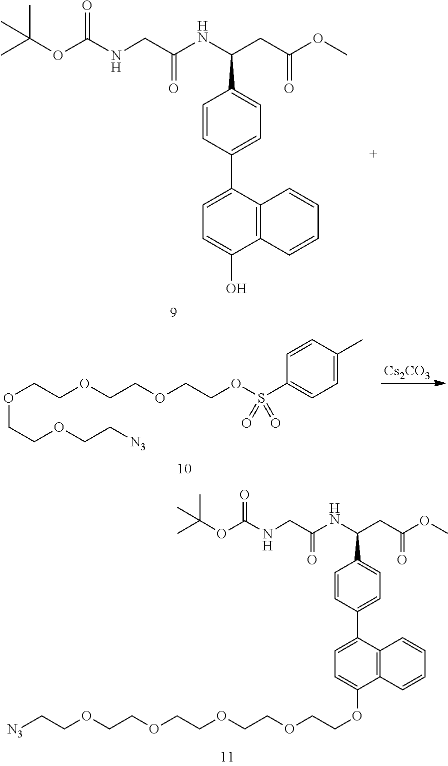

To a solution of compound 9 (1.61 g, 3.364 mmol, 1.0 equiv.) and compound 10 (1.75 g, 4.205 mmol, 1.25 equiv.) in anhydrous DMF (10 mL) was added cesium carbonate (2.19 g, 6.728 mmol, 2.0 equiv.) at room temperature. The reaction mixture was kept at 50° C. for 2 hours. The reaction mixture was quenched with water (20 mL) and was extracted with ethyl acetate (3×10 mL). The organic phase was combined, dried over anhydrous Na₂SO₄, and concentrated. Compound 11 was purified by CombiFlash®, and was eluted with 2-4% MeOH in DCM. LC-MS: calculated [M+H]+ 724.35, found 724.60.

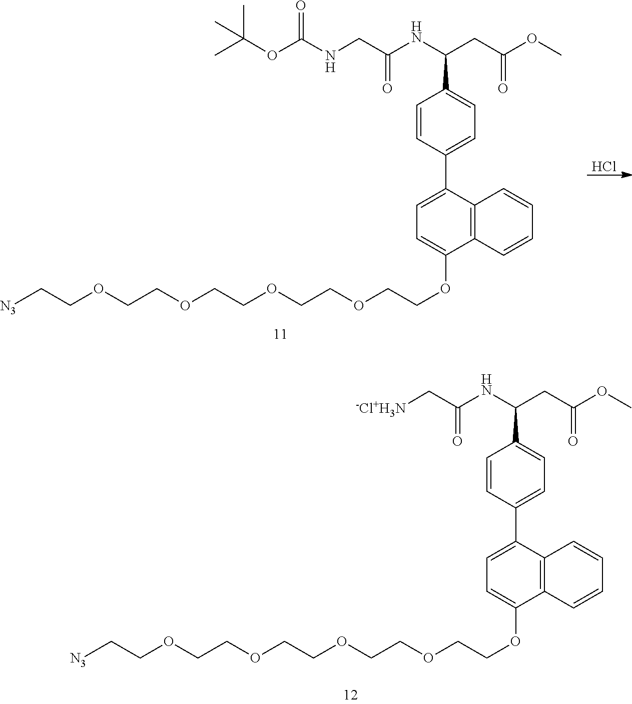

To a solution of compound 11 (1880 mg, 2.597 mmol, 1.0 equiv.) in anhydrous dioxane (3 mL) was added HCl in dioxane (3.25 mL, 12.986 mmol, 5.0 equiv.) at room temperature. The reaction mixture was kept at room temperature for 2 hours. The solvent was removed and compound 12 was used directly without purification. LC-MS: calculated [M+H]+ 624.30, found 624.41.

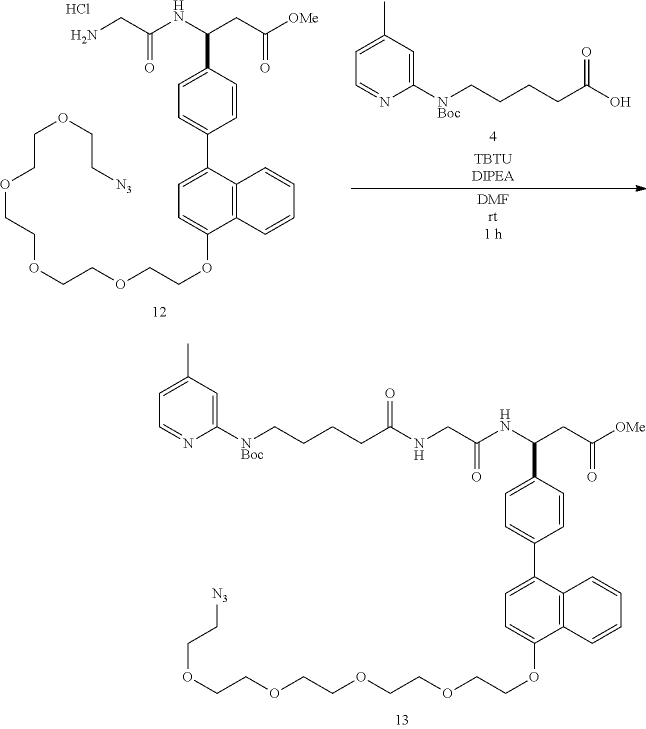 [SMILES: COC(=O)C[C@H](NC(=O)CN)c1ccc(-c2ccc(OCCOCCOCCOCCOCCN=[N+]=[N-])c3ccccc23)cc1 ; Cl ; Cc1ccnc(N(CCCCC(=O)O)C(=O)OC(C)(C)C)c1 ; COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1]

To a solution of compounds 12 (0.10 g) and 4 (0.049 g) in DMF was added TBTU (0.058 g) and then DIPEA (0.079 mL) at room temperature. Reaction was stirred for 1 hour until full conversion was observed by LC-MS. The reaction mixture was then quenched with NaHCO₃ (10 mL). The product was extracted with EtOAc (3×15 mL) and then washed with water (3×10 mL) and brine (10 mL). The combined organic phases were dried over Na₂SO₄, filtered, and concentrated. The residue was purified by CombiFlash® using silica gel as the stationary phase with a gradient of 0-20% MeOH in DCM (0-70%), in which compound 13 eluted at 23% B. The product was concentrated under vacuum to provide a clear colorless oil (0.088 g, yield 63.6%.)

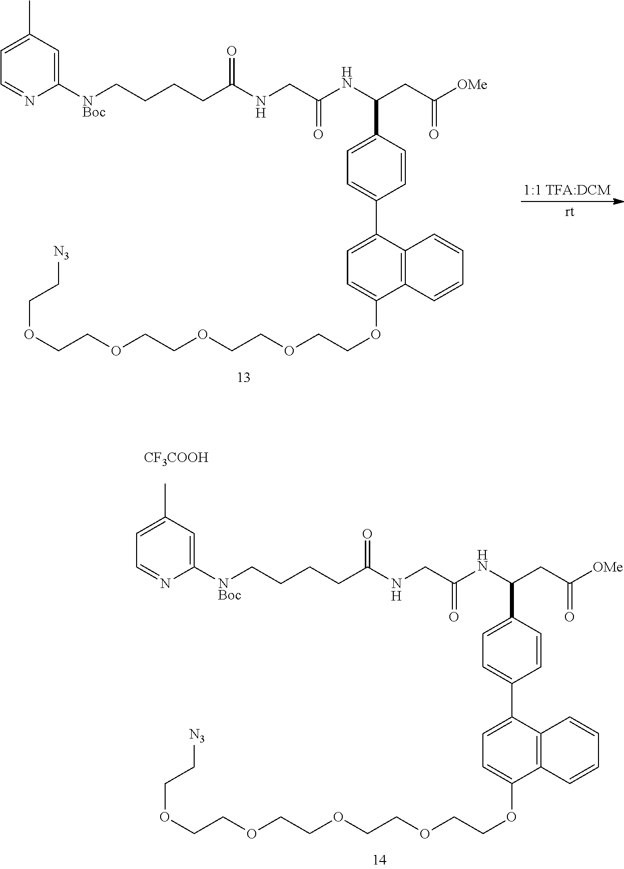 [SMILES: COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1 ; "CF3COOH" ; COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1]

To a solution of compound 13 (0.088 g) in DCM was added TFA (0.22 mL) at room temperature. The reaction mixture was stirred at room temperature for 5 hours until full conversion was confirmed via LC-MS. The reaction mixture was azeotroped with PhMe and concentrated under vacuum. No isolation was necessary. Concentration provided compound 14 as a clear colorless oil (0.10 g, yield 113%.) LC-MS: calculated [M+H]+ 814.41 m/z, observed 814.63 m/z.

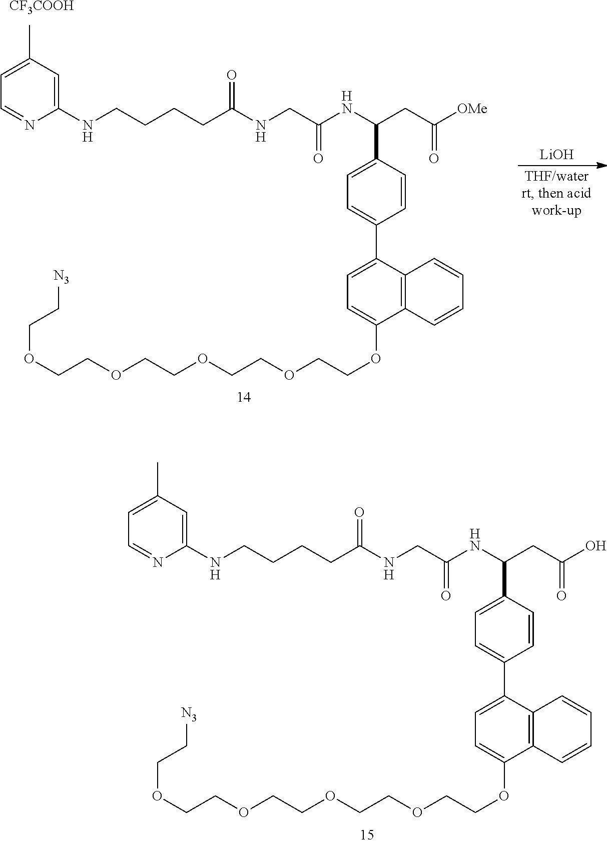 [SMILES: "CF3COOH" ; COC(=O)C[C@H](NC(=O)CNC(=O)CCCCNc1cc(C)ccn1)c1ccc(-c2ccc(OCCOCCOCCOCCOCCN=[N+]=[N-])c3ccccc23)cc1 ; "LiOH" ; "THF/waterrt, then acidwork-up" ; Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 14 (0.10 g) in 1:1 THF/water was added LiOH (0.0078 g) at room temperature under normal atmosphere. The reaction mixture was stirred at room temperature until full conversion was observed by LC-MS. After 4 hours, the reaction mixture was acidified with 6 N HCl to a pH of approximately 3. The product was extracted with 20% CF₃CH₂OH/DCM (3×15 mL). The combined organic phases were dried over Na₂SO₄, filtered, and concentrated, providing compound 15 as a light yellow solid (0.104 g, yield 119%.) LC-MS: calculated [M+H]+ 800.39 m/z, observed 800.76 m/z.

### Example 4. Synthesis of Lipid PK/PD Modulator Precursors

### Synthesis of LP1-p

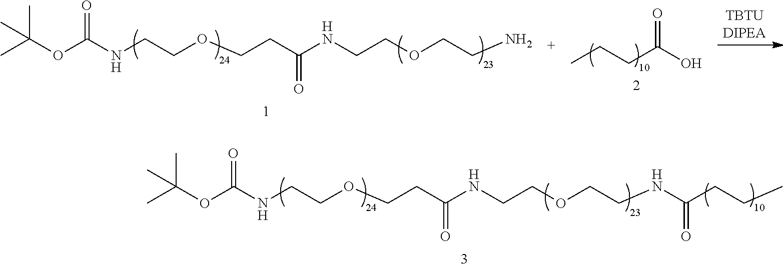

To a solution of compound 1 (2630 mg, 1.142 mmol, 1.0 equiv.), compound 2 (428 mg, 1.256 mmol, 1.1 equiv.), and diisopropylethylamine (0.597 mL, 3.427 mmol, 3.0 equiv.) in anhydrous DMF (10 mL) was added TBTU (440 mg, 1.371 mmol, 1.2 equiv.) at room temperature. The reaction mixture was kept at room temperature for 2 hours and then concentrated. Compound 3 was purified by CombiFlash® eluting with 12-17% MeOH in DCM. LC-MS: calculated [M+4H]+/4 656.66, found 656.65.

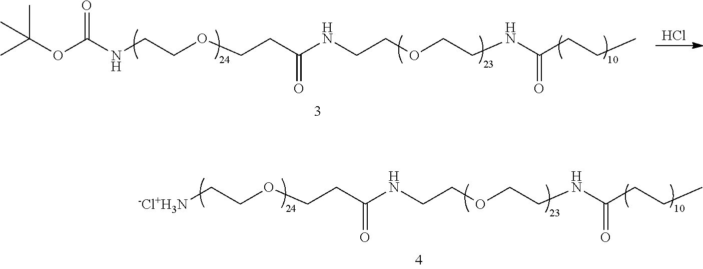

To solid of compound 3 (1150 mg, 0.438 mmol, 1.0 equiv.) was added HCl solution in dioxane (5.478 mL, 21.910 mmol, 50 equiv.) at room temperature. The reaction mixture was kept at room temperature for 30 minutes and then concentrated. Compound 4 was used directly without further purification. LC-MS: calculated [M+3H]+/3 841.88, found 842.56, calculated [M+4H]+/4 631.66, found 632.41.

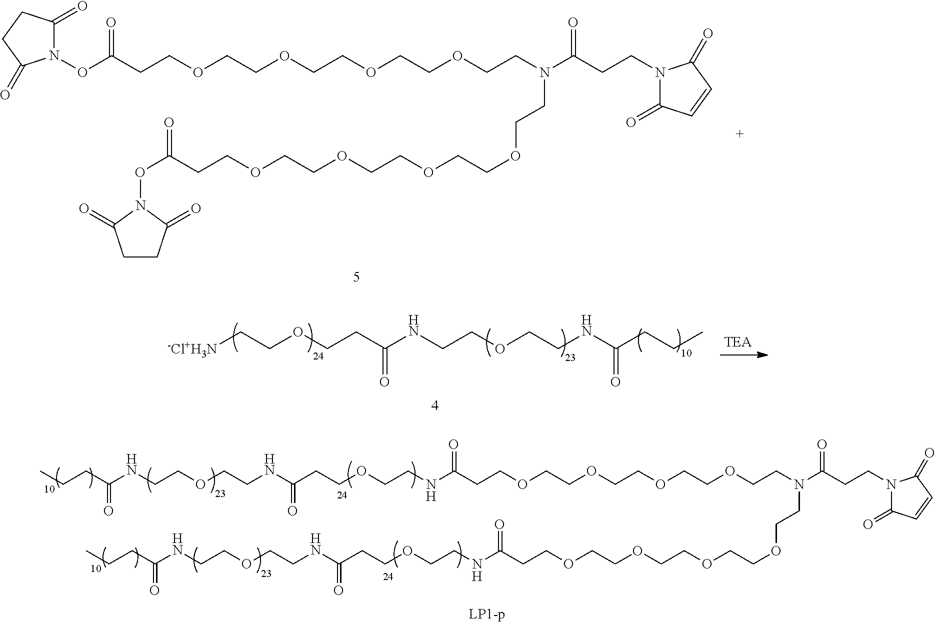

To a solution of compound 5 (175 mg, 0.203 mmol, 1.0 equiv.) and compound 4 (1095 mg, 0.427 mmol, 2.1 equiv.) in anhydrous DCM (10 mL) was added TEA (0.144 mL, 1.018 mmol, 5.0 equiv.) at room temperature. The reaction mixture was kept at room temperature for 3 hours and the solvent was removed under vacuum. LP1-p was purified by CombiFlash® eluting with 10-17% MeOH in DCM. LC-MS: calculated [M+6H]+/6 946.60, found 947.10, calculated [M+7H]+/7 811.51, found 811.35.

### Synthesis of LP5-p

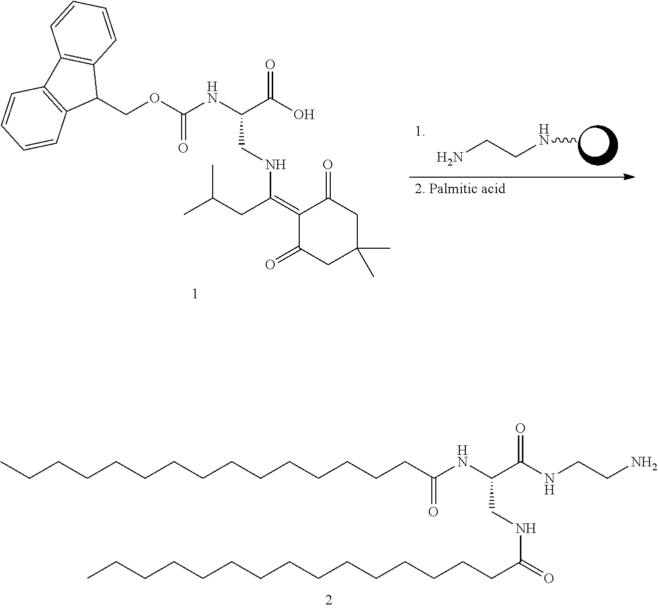

Compound 1 (105 mg, 0.198 mmol) in DMF was treated with TBTU (4 equiv.) and agitated for 5 minutes. DIEA (8 equiv.) was subsequently added and the mixture was added to 1 molar eq. of ethylamine diamine on pre-swelled 2-chlorotrityl resin. After agitation for 30 minutes the resin was washed three times with DMF and then treated with 2% hydrazine in DMF for 10 minutes. Coupling of palmitic acid (202 mg, 0.789 mmol) was repeated using the same procedure as the coupling of compound 1. Upon completion, the resin was washed with 3 portions of DCM and treated with a 1% solution of TFA in DCM for 10 minutes. TFA treatment was repeated and the resin was washed with 3 portions of DCM. All volatiles were removed and the crude compound 2 was used without further purification. Yield 126 mg (81%).

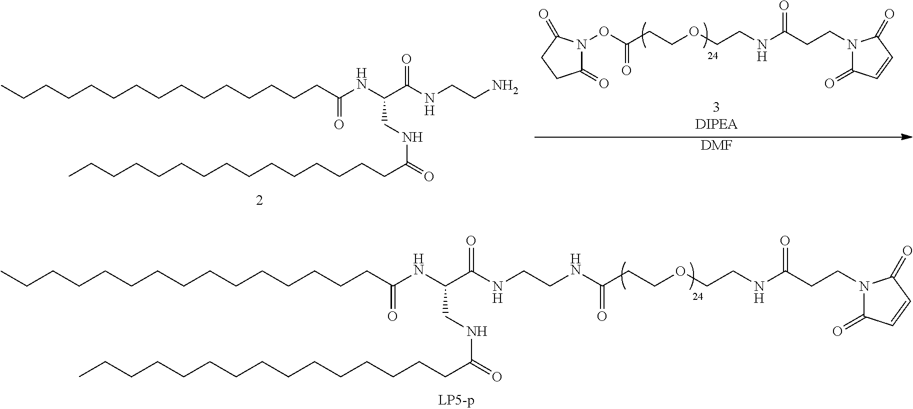

To a mixture containing compound 2 (23 mg, 37 μmol) and DIEA (14.1 μL, 81 μmol) in DMF (1 mL) was added NHS-PEG24-MAL (compound 3, 61.5 mg, 0.0441 mmol) and the reaction mixture was stirred for 30 minutes. Upon completion crude LP5-p was dry loaded onto silica and isolated eluting a gradient of MeOH in DCM. Yield 15 mg (21%).

### Synthesis of LP28-p

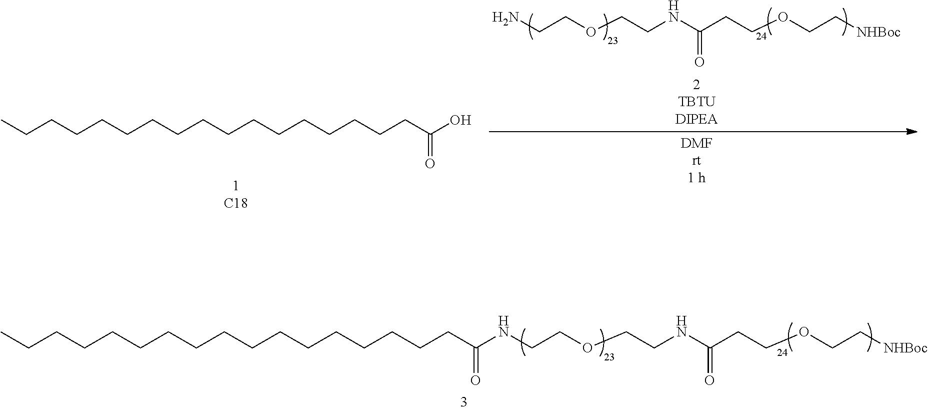

To a solution of compounds 1 (80 mg) and 2 (60.2 mg) in DMF was added TBTU (90.3 mg) and then DIPEA (0.147 mL) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (0-80%, isocratic, and then to 100%) over 20-30 minutes, in which compound 3 eluted at 68% B. Compound 3 was concentrated under vacuum to provide a white oily residue. LC-MS: calculated [M+H]+ 2567.65 m/z, observed 130.178 (+2/2, +H₂O) m/z.

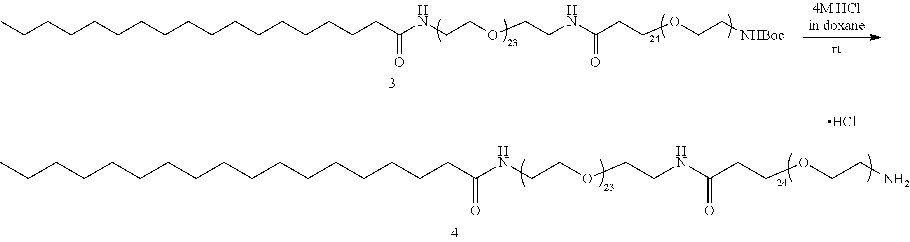

To compound 3 (100.4 mg) was added 4 M HCl/dioxane (14.3 mg) at room temperature. The reaction mixture was stirred at room temperature. The reaction mixture was stirred overnight until full conversion was confirmed via LC-MS. The reaction mixture was azeotroped with PhMe and concentrated under vacuum overnight to provide an compound 4 as an oil. LC-MS: calculated [M+H]+2467.60 m/z, observed 1243.32 m/z.

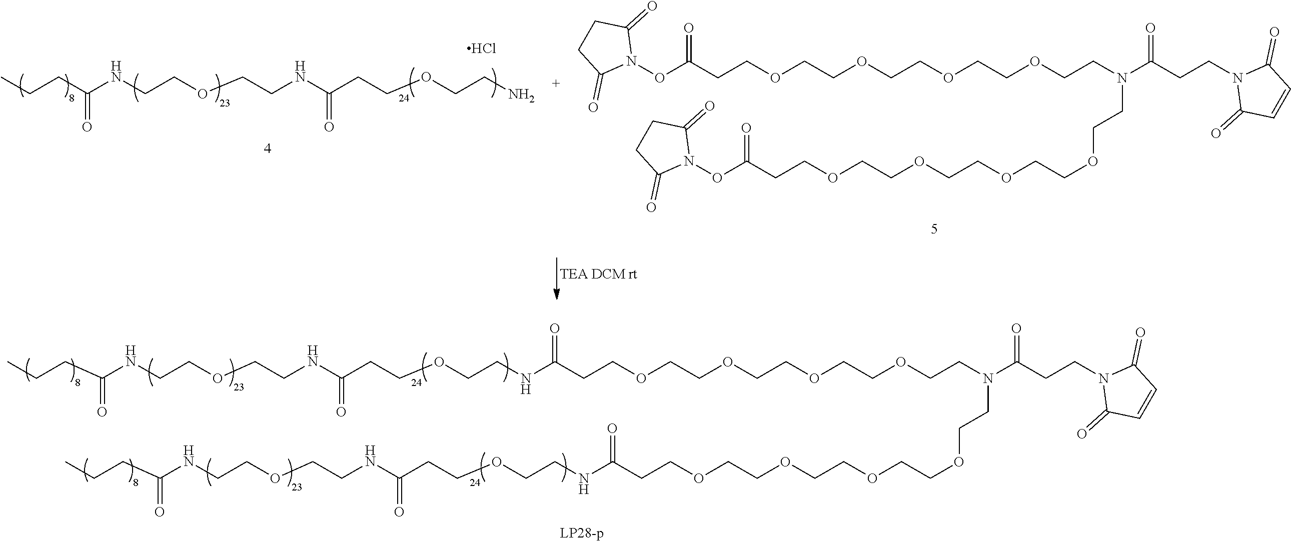

A solution of compound 4 (97.9 mg) and TEA (0.016 mL) in anhydrous DCM was prepared and stirred under sparging nitrogen atmosphere. Compound 5 (15.8 mg) was then added to the reaction mixture. The reaction mixture was stirred at room temperature until full conversion was observed by LC-MS. The reaction mixture was then directly concentrated. The residue was purified by CombiFlash® using silica gel as the stationary phase and was eluted with a gradient of 0-20% MeOH in DCM (0-100% B). LP28-p eluted at 67% B. LC-MS: calculated [M+H]+ 5562.48 m/z, observed 1409.68 (+4/4, +H₂O) m/z.

### Synthesis of LP29-p

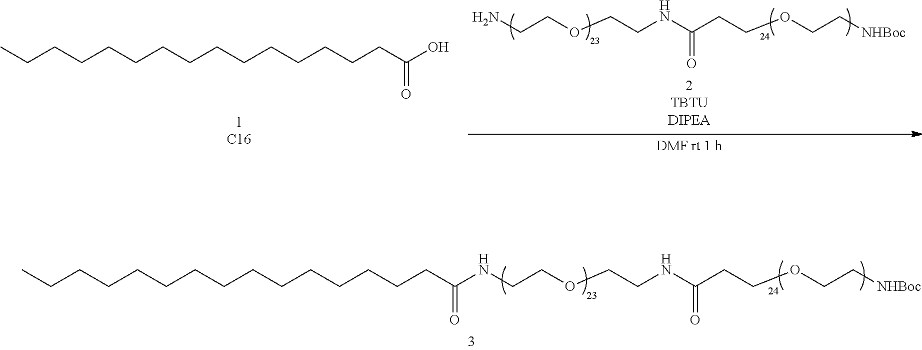

To a solution of compounds 1 (40 mg) and 2 (334 mg) in DMF was added TBTU (50.1 mg) and then DIPEA (0.082 mL) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (0-80%) over 20-30 minutes, in which compound 3 eluted at 71% B. Compound 3 was concentrated under vacuum to provide a white oily residue. LC-MS: calculated [M+H]+ 2539.62 m/z, observed 1288.21 (+2/2, +H₂O) m/z.

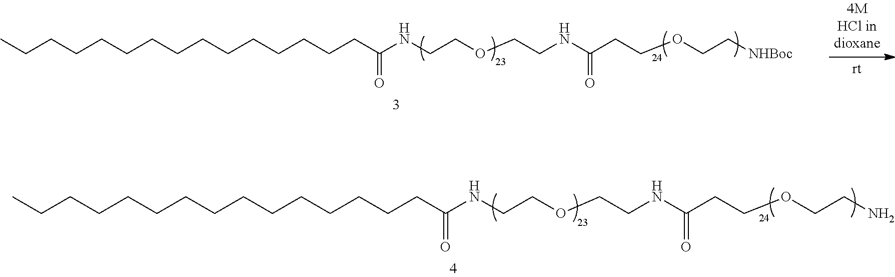

To compound 3 (147 mg) was added 4 M HCl/dioxane (21.2 mg) at room temperature. The reaction mixture was stirred at room temperature. The reaction mixture was stirred overnight until full conversion was confirmed via LC-MS. The reaction mixture was azeotroped with PhMe and concentrated under vacuum overnight to provide compound 4 as an oil. LC-MS: calculated [M+H]+ 2439.57 m/z, observed 611.16 (+4/4) m/z.

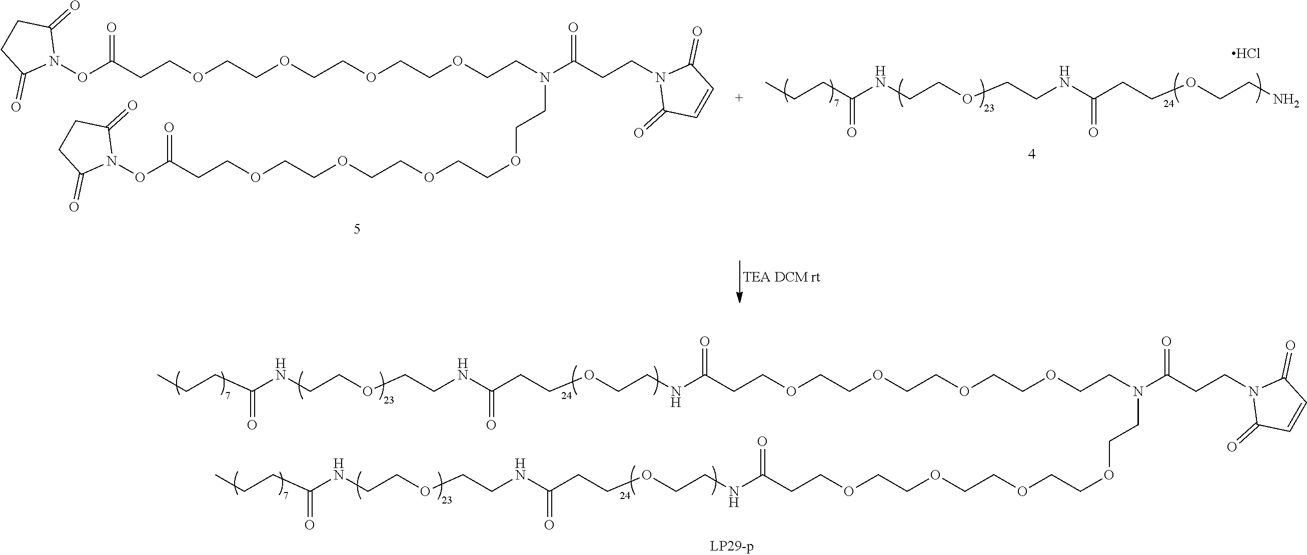

A solution of compound 4 (143 mg) and TEA (0.024 mL) in anhydrous DCM was prepared and stirred under sparging nitrogen atmosphere. Compound 5 (23.4 mg) was then added to the reaction mixture. The reaction mixture was stirred at room temperature until full conversion was observed by LC-MS.

The reaction mixture was then directly. The residue was purified by CombiFlash® using silica gel as the stationary phase and eluting with a gradient of 0-20% MeOH in DCM (0-100% B). LP29-p eluted at 54% B. LC-MS: calculated [M+H]+ 5506.42 m/z, observed 1854.41 (+3/3, +H₂O) m/z.

### Synthesis of LP33-p

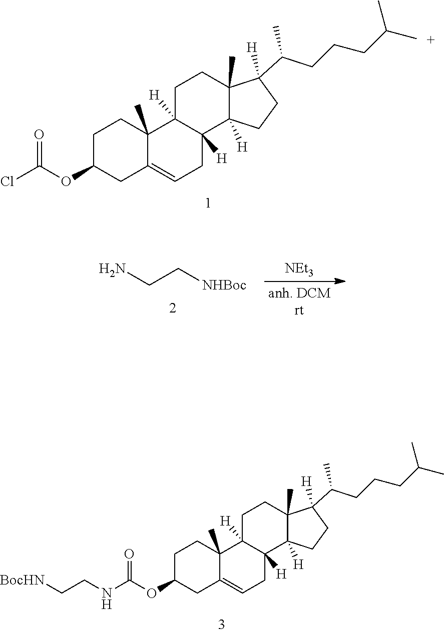

To a solution of compounds 1 (2.00 g, 4.45 mmol) and 2 (1.07 g, 6.68 mmol) in anhydrous DCM, NEt₃ (1.86 mL, 13.4 mmol) was added at room temperature. Reaction was stirred until full conversion was observed by LC-MS. The reaction mixture was then directly concentrated. The residue was purified by CombiFlash® using silica gel as the stationary phase with a gradient of 0-20% MeOH in DCM (0-100%) over 45 minutes, in which compound 3 eluted at 8% B. Compound 3 was concentrated to provide a white solid. LC-MS: calculated [M+H]+ 573.46 m/z, observed 573.60 m/z.

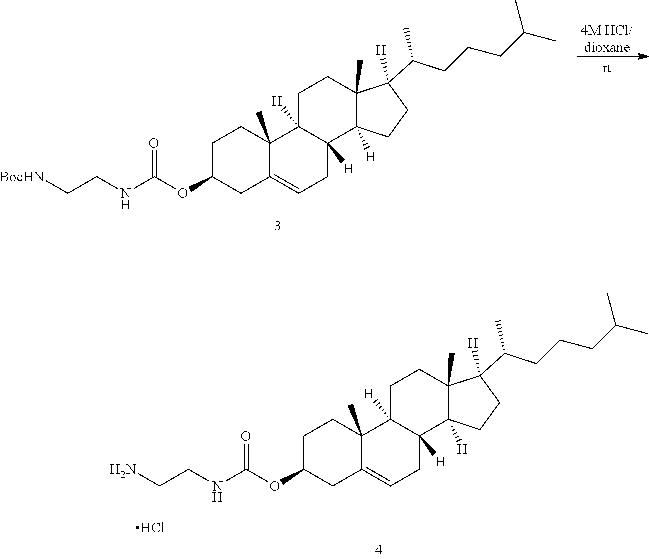 [SMILES: CC(C)CCC[C@@H](C)[C@H]1CC[C@H]2[C@@H]3CC=C4C[C@@H](OC(=O)NCCNC(=O)OC(C)(C)C)CC[C@]4(C)[C@H]3CC[C@]12C ; "4M HCl/dioxane" ; CC(C)CCC[C@@H](C)[C@H]1CC[C@H]2[C@@H]3CC=C4C[C@@H](OC(=O)NCCN)CC[C@]4(C)[C@H]3CC[C@]12C ; Cl]

To compound 3 (317 mg, 0.553 mmol) was added 4 M HCl/dioxane (1.383 mL) at room temperature. The reaction mixture was stirred at room temperature. The reaction mixture was stirred overnight until full conversion was confirmed via LC-MS. The reaction mixture was concentrated under high-vacuum overnight to provide compound 4 as a clear and colorless greasy residue. LC-MS: calculated [M+H]+ 473.40 m/z, observed 473.58 m/z.

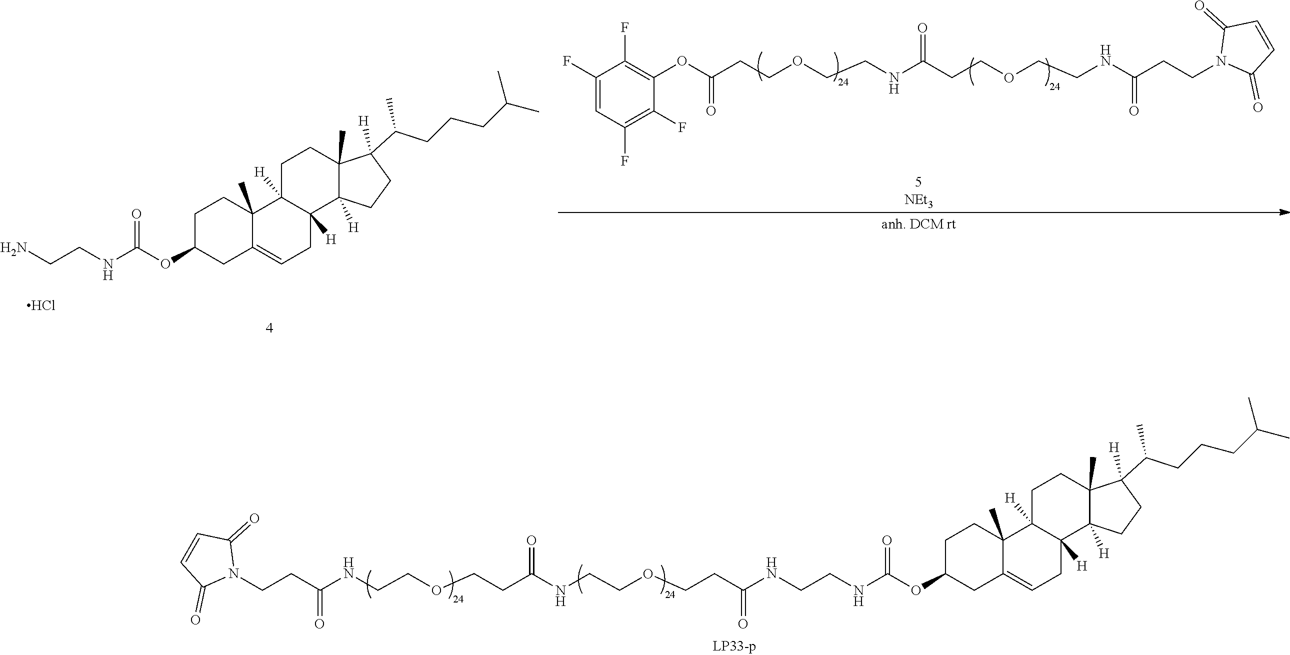

To a solution of compounds 4 (282 mg, 0.553 mmol) and 5 (1.35 g, 0.526 mmol) in anhydrous DCM under N₂(g), NEt₃ (0.386 mL) was added. The reaction mixture was stirred until full conversion was observed by LC-MS. The reaction mixture was then directly concentrated. The residue was purified by CombiFlash® using silica gel as the stationary phase with a gradient of 0-20% MeOH in DCM (0-100%) over 45 minutes, in which LP33-p eluted at 46% B. LP33-p was concentrated to provide a white solid. LC-MS: calculated [M+H]+ 2879.76 m/z, observed 960.98 (+3/3) m/z.

### Synthesis of LP38-p

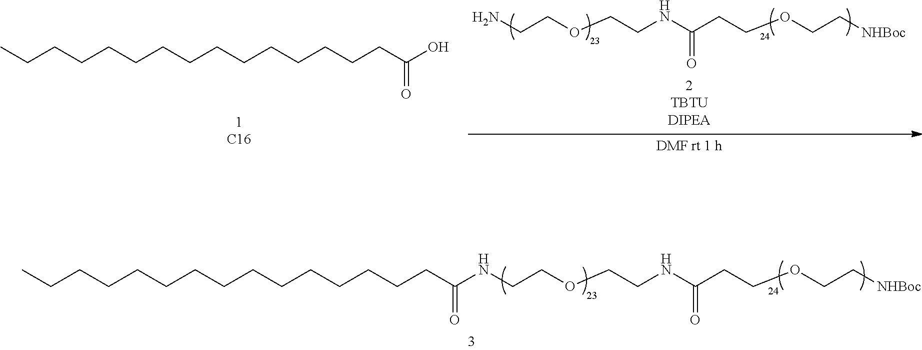

To a solution of compounds 1 (35 mg) and 2 (299 mg) in DMF was added TBTU (43.8 mg) and then DIPEA (0.071 mL) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (0-100%) over 20-30 minutes, in which compound 3 eluted at 56% B. Compound 3 was concentrated under vacuum to provide a white oily residue. LC-MS: calculated [M+H]+ 2539.62 m/z, observed 1288.07 (+2/2, +H₂O) m/z.

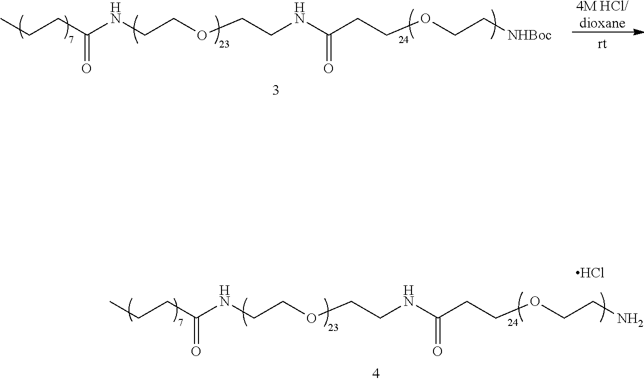

To compound 3 (186 mg) was added 4 M HCl/dioxane (26.7 mg) at room temperature. The reaction mixture was stirred at room temperature. The reaction mixture was stirred overnight until full conversion was confirmed via LC-MS. The reaction mixture was azeotroped with PhMe and concentrated under vacuum overnight to provide compound 4 as an oil. LC-MS: calculated [M+H]+ 2439.57 m/z, observed 1220.97 (+2/2) m/z.

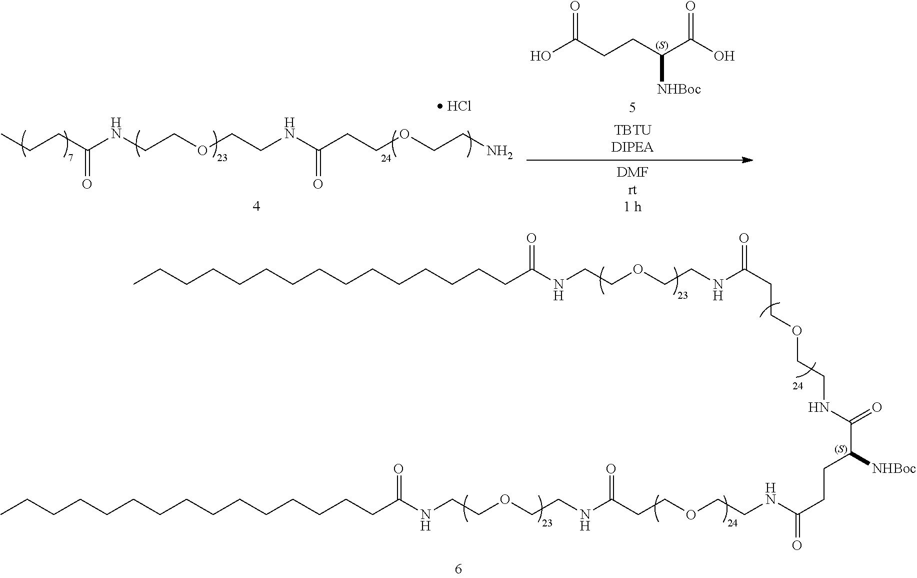

To a solution of compound 4 (181 mg), TBTU (24 mg), and DIEA (0.033 mL) in DMF was added compound 5 (8.7 mg) at room temperature. Reaction was stirred until full conversion was observed by LC-MS. The reaction mixture was then directly concentrated. The residue was purified by CombiFlash® using silica gel as the stationary phase with a gradient of 0-20% MeOH in DCM (0-100%) over 20-30 minutes, in which compound 6 eluted at 65% B. Compound 6 was concentrated under vacuum to provide a white oily residue. LC-MS: calculated [M+H]+ 5089.22 m/z, observed 1036.24 (+5/5, +H₂O) m/z.

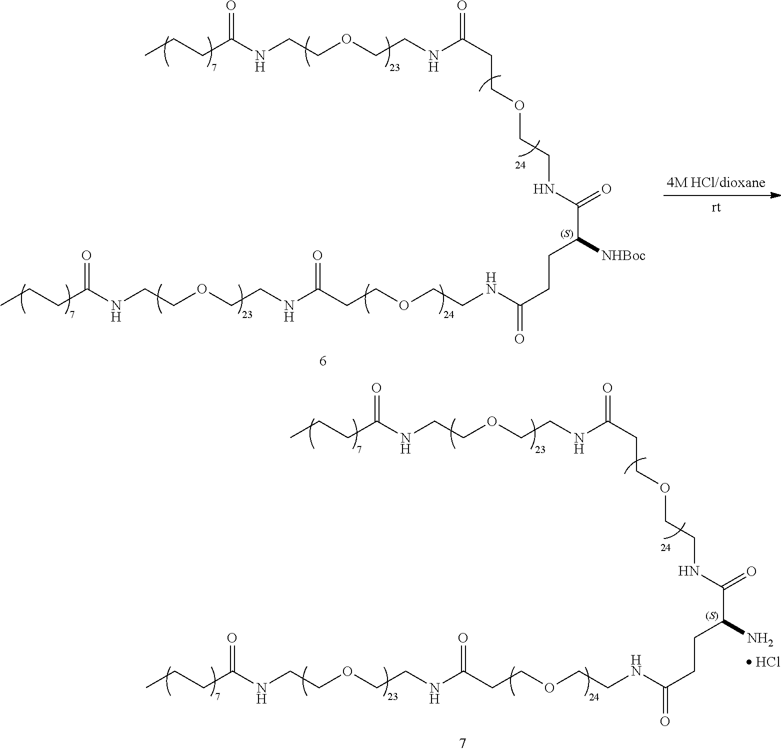

To compound 6 (130 mg) was added 4 M HCl/dioxane (9.3 mg) at room temperature. The reaction mixture was stirred at room temperature. The reaction mixture was stirred overnight until full conversion was confirmed via LC-MS. The reaction mixture was azeotroped with PhMe and concentrated under vacuum overnight to provide compound 7 as an oil. LC-MS: calculated [M+H]+ 4989.17 m/z, observed 1248.58 (+4/4) m/z.

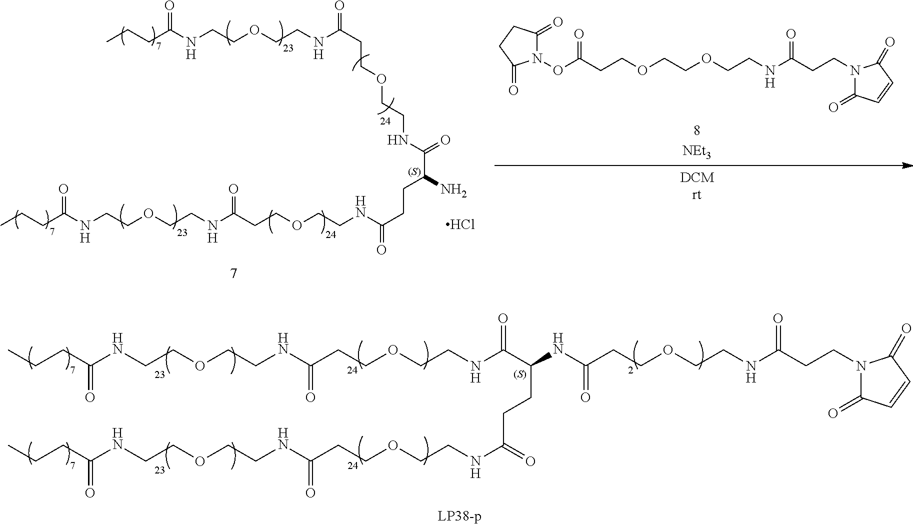

A solution of compound 7 (128 mg) and NEt₃ (0.018 mL) in anhydrous DCM under sparging N₂(g) was prepared at room temperature. Compound 8 (10.3 mg) was then added slowly. The reaction mixture was allowed to stir until full conversion was observed by LC-MS. The reaction mixture was then directly concentrated. The residue was purified by CombiFlash® using silica gel as the stationary phase with a gradient of 0-20% MeOH in DCM (0-100%) over 30 minutes, in which LP38-p eluted at 100% B. LP38-p was concentrated to provide a white solid. LC-MS: calculated [M+H]+ 5299.28 m/z, observed 1786.62 (+3/3, +H₂O) m/z.

### Synthesis of LP39-p

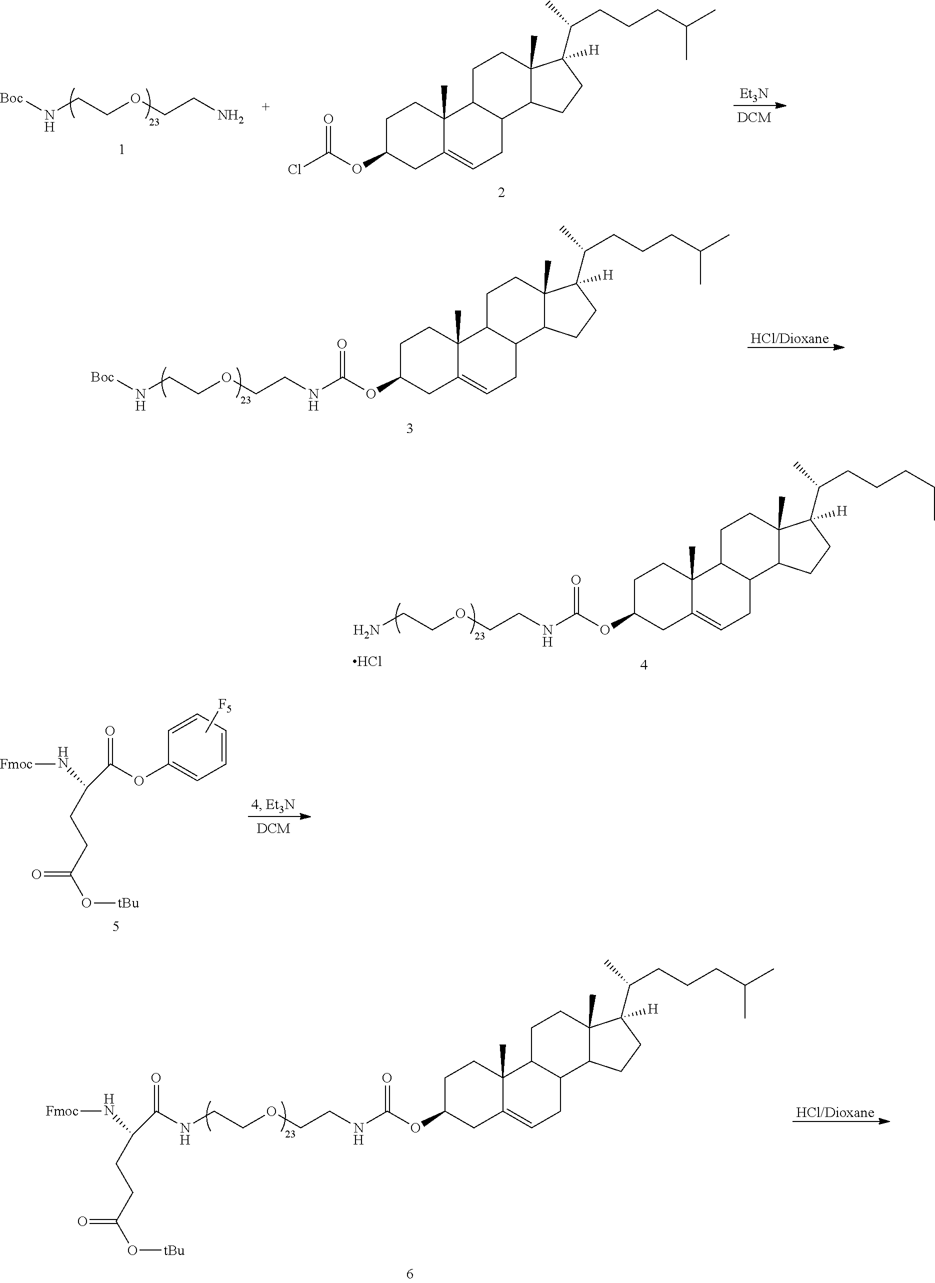

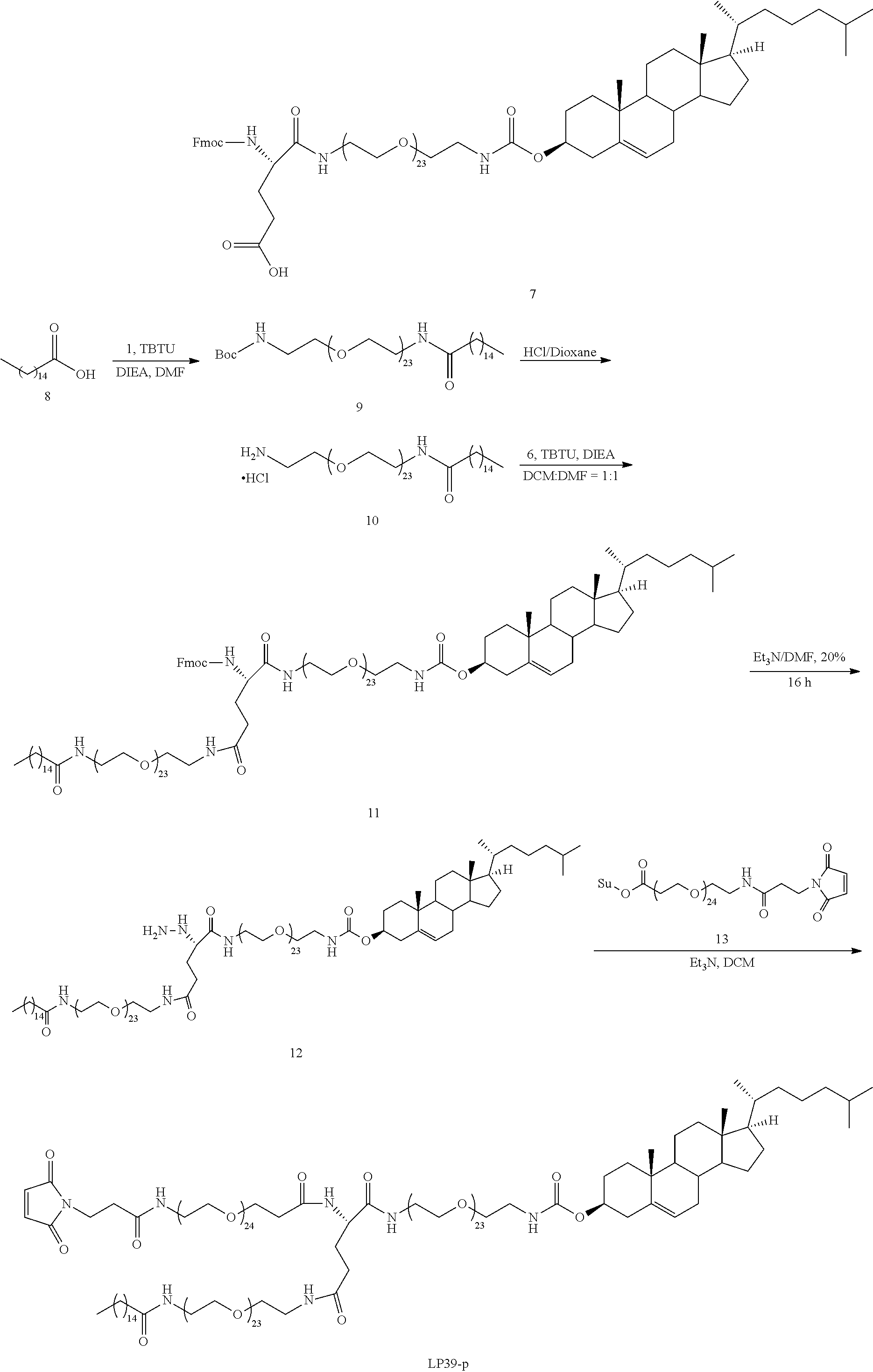

Boc-protected PEG₂₃-amine 1 (Quanta Biodesign Limited, 200 mg, 0.17 mmol) was stirred with cholesterol chloroformate 2 (77 mg, 0.17 mmol) and Et₃N (48 μL, 0.341 mmol) in 5 mL of DCM for 1.5 h. The solvent was removed under vacuum, the residue was mixed with SiO₂ (1 g) and loaded on a CombiFlash®. Compound 3 was purified using the system 0-20% MeOH in DCM, gradient 0-80%, 40 minutes. Calculated MW 1586.09, M+18=1604.09, (M+2×18)/2=811.05 Found: MS (ES, pos): 1603.55 [M⁺NH₄]⁺, 811.07 [M⁺²NH₄]²⁺.

Product 3 was Boc-deprotected and the resulting hydrochloride salt 4 (62 mg, 0.04 mmol) was stirred with pentafluorophenyl ester 5 (24 mg, 0.04 mmol) and Et₃N (14 μL, 0.1 mmol) in DCM (5 mL) for 1.5 hours. The solvent was removed under vacuum, the residue was mixed with SiO₂ (400 mg) and loaded on a CombiFlash®. The product 6 was purified using the system 0-20% MeOH in DCM, gradient 0-70%, 30 minutes. Yield 57 mg. Calculated MW 1893.44, M+18=1911.44, (M+2×18)/2=964.72 Found: MS (ES, pos): 1911.00 [M+NH₄]⁺, 964.46 [M+2NH₄]²⁺.

Product 6 was treated with 4M HCl in dioxane (10 mL) for 4 hours at room temperature. The solvent was removed under vacuum, toluene was evaporated 2 times from the residue, product 7 was dried and used directly in the next step.

Solid TBTU (50 mg, 0.156 mmol) was added to a solution of Boc-protected PEG₂₃-amine 1 (Quanta Biodesign Limited, 152 mg, 0.13 mmol), palmitic acid 8 (33 mg, 0.13 mmol), and DIEA (68 μL, 0.39 mmol) in DMF (9 mL). The reaction mixture was sonicated to dissolve solids and stirred for 16 hours at room temperature. The solvent was removed under vacuum, toluene was evaporated twice from the residue, the residue was dissolved in chloroform (50 mL), washed with NaHCO₃ (2×10 mL) and brine (10 mL). Compound 9 was dried (Na₂SO₄), concentrated under vacuum, and purified on CombiFlash® (SiO₂) using the system DCM: 20% MeOH in DCM, gradient 0-80%, 20 min. Calculated MW 1411.85, M+18=1429.85, (M+1+18)/2=715.43 Found: MS (ES, pos): 1429.24 [M+NH₄]⁺, 715.41 [M+H+NH₄]²⁺.

9 was Boc-deprotected with HCl/dioxane solution and compound 10 was used directly in the next step.

The derivative 7 (60 mg, 0.028 mmol) was stirred with hydrochloride salt 10 (42 mg, 0.03 mmol), TBTU (11 mg, 0.034 mmol) and DIEA (18 μL, 0.1 mmol) in DCM:DMF=1:1 (8 mL) for 3 hours. The solvent was removed under vacuum, toluene was evaporated 2 times from the residue, and the solid was suspended in CHCl₃ (50 mL). The suspension was washed twice with 2% NaHCO₃ and brine. Following concentration under vacuum, the product 11 was purified on CombiFlash® (0-20% MeOH in DCM, gradient 0-70%, 35 minutes)

The product 11 (51 mg, 0.0162 mmol) was stirred with Et₃N in DMF (20%, 3 mL) for 16 hours, the solvent with Et₃N was removed under vacuum, toluene was evaporated 3 times from the residue to obtain deprotected amine 12. Calculated MW 2908.81, (M+1+18)/2=1463.91, (M+1+18×2)/3=981.94 Found: MS (ES, pos): 1463.69 [M+H+NH₄]²⁺, 981.99 [M+H+2NH₄]³⁺.

Amine 12 (47 mg, 0.0162 mmol) was stirred with the mixture of NHS ester 13 (21 mg, 0.0147 mmol) and Et₃N (6 μL, 0.041 mmol) in DCM (4 mL) for 16 hours. The solvent was removed under vacuum, and the product LP39-p was purified on CombiFlash® using the system 0-20% MeOH in DCM, gradient 0-100%, 40 minutes. Calculated MW 4188.28, (M+2+18)/3=1402.76, (M+3+18×2)/4=1052.32 Found: MS (ES, pos): 1402.71 [M+2H+NH₄]³⁺, 1052.32 [M+3H+NH₄]⁴⁺.

### Synthesis of LP41-p

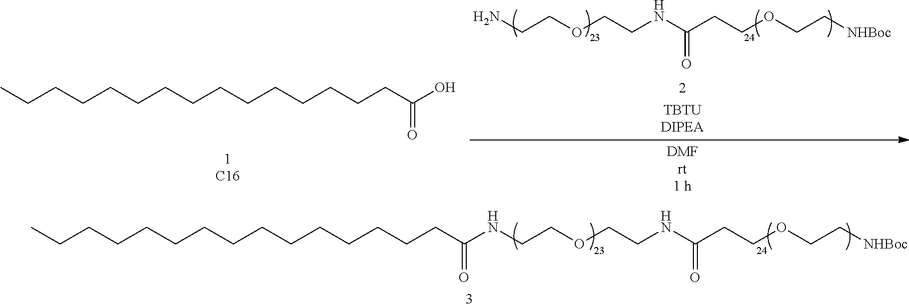

To a solution of compound 1 (40.0 mg), TBTU (50.1 mg), and DIEA (0.098 mL) in DMF was added compound 2 (298 mg) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (10-100% B) over 20-30 minutes, in which compound 3 eluted at 43% B. Compound 3 was concentrated under vacuum to provide a white oily residue. LC-MS: calculated [M+H]+ 2539.62 m/z, observed 1287.83 (+2/2, +H₂O) m/z.

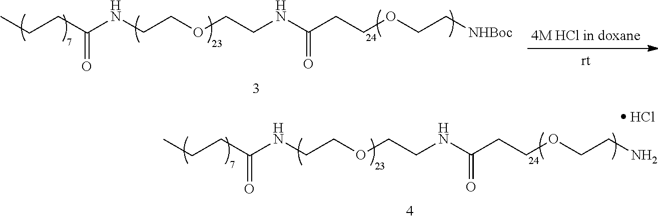

To compound 1 (260 mg) was added 4 M HCl/dioxane (37.4 mg) at room temperature. The reaction mixture was stirred at room temperature. The reaction mixture was stirred overnight until full conversion was confirmed via LC-MS. The reaction mixture was azeotroped with PhMe and concentrated under vacuum overnight to provide compound 4 as an oil. LC-MS: calculated [M+H]+ 2439.57 m/z, observed 1220.61 (+2/2) m/z.

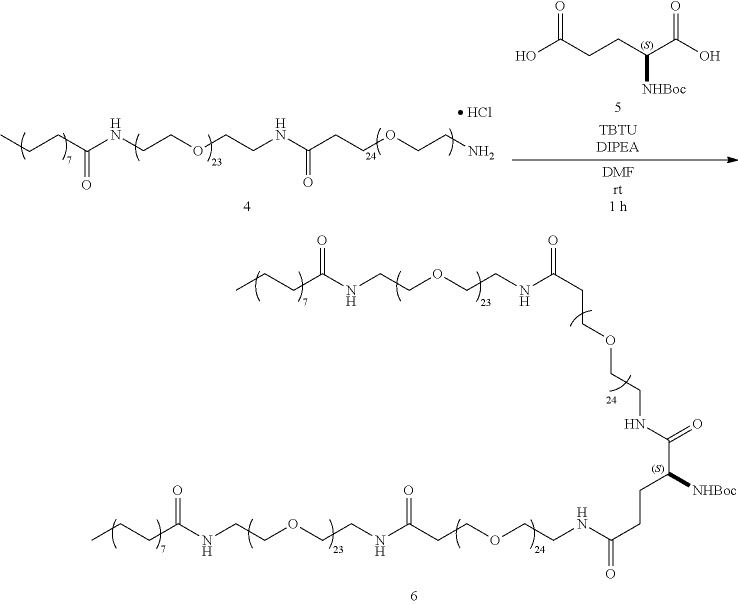

To a solution of compound 4 (253 mg), TBTU (36.1 mg), and DIEA (0.045 mL) in DMF was added compound 5 (11.9 mg) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (10-30, 35, then 100%) over 30 minutes, in which compound 6 eluted at 35% B. Compound 6 was concentrated under vacuum to provide a white oily residue. LC-MS: calculated [M+H]+ 5089.22 m/z, observed 1715.43 (+3/3, +H₂O) m/z.

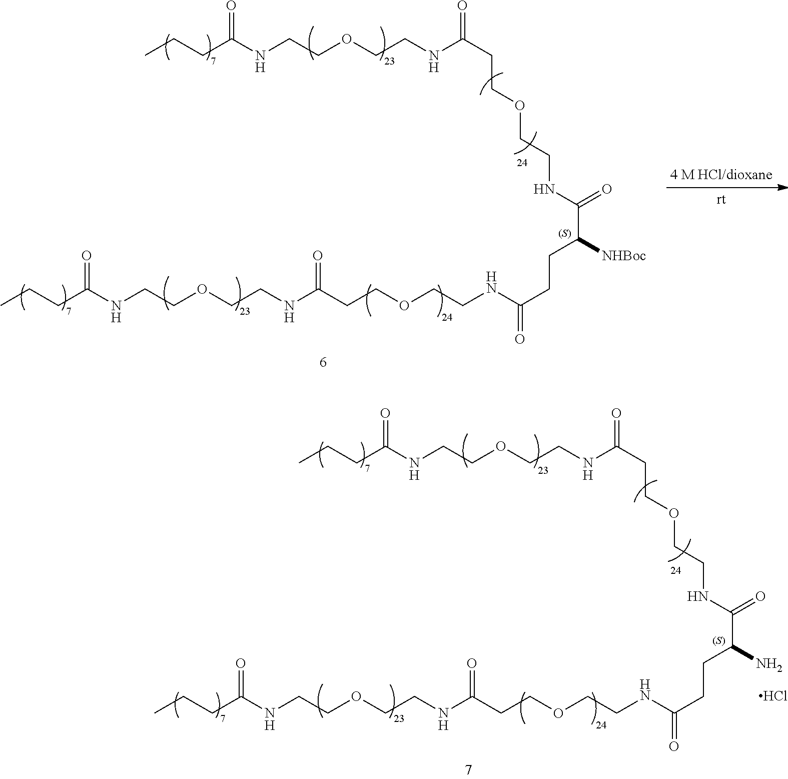

To compound 6 (35.4 mg) was added 4 M HCl/dioxane (2.5 mg) at room temperature. The reaction mixture was stirred at room temperature. The reaction mixture was stirred overnight until full conversion was confirmed via LC-MS. The reaction mixture was azeotroped with PhMe/MeOH and concentrated under high-vacuum overnight to provide compound 7 as an oil. LC-MS: calculated [M+H]+ 4989.17 m/z, observed 1676.42 (+HCl, +3/3) m/z.

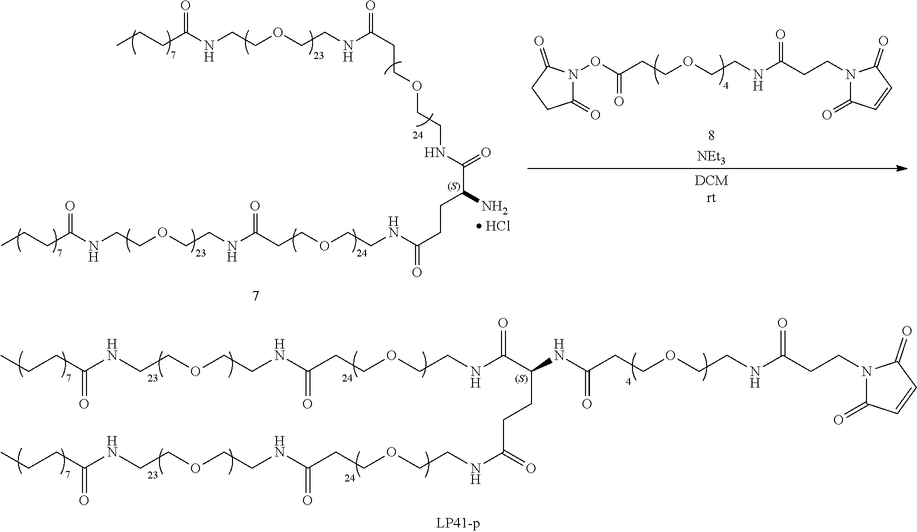

A solution of compound 7 (35 mg) and NEt₃ (0.005 mL) in anhydrous DCM under sparging N₂(g) was prepared at room temperature. Compound 8 (3.2 mg) was then added slowly. The reaction mixture was allowed to stir until full conversion was observed by LC-MS. The reaction mixture was then directly concentrated. The residue was purified by CombiFlash® using silica gel as the stationary phase with a gradient of 0-20% MeOH/DCM (10 to 30%, 40%, 50%, 70%, then 100% B) over 30 minutes, in which LP41-p eluted at 100% B. LC-MS: calculated [M+H]+ 5837.84 m/z, observed 1079.90 (+5/5) m/z.

### Synthesis of LP42-p

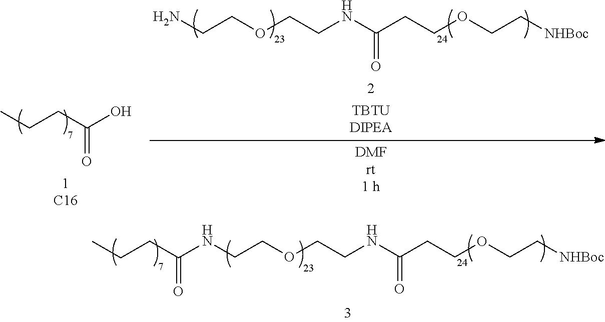

To a solution of compound 1 (40 mg), TBTU (50.1 mg), and DIEA (0.098 mL) in DMF was added compound 2 (298 mg) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (10-100% B) over 20-30 minutes, in which compound 3 eluted at 43% B. Compound 3 was concentrated under vacuum to provide a white oily residue. LC-MS: calculated [M+H]+ 2539.62 m/z, observed 1287.83 (+2/2, +H₂O) m/z.

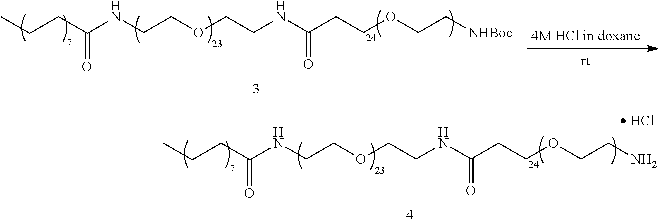

To compound 3 (260 mg) was added 4 M HCl/dioxane (37.4 mg) at room temperature. The reaction mixture was stirred at room temperature. Reaction was stirred overnight until full conversion was confirmed via LC-MS. The reaction mixture was azeotroped with PhMe and concentrated under vacuum overnight to provide compound 4 as an oil. LC-MS: calculated [M+H]+ 2439.57 m/z, observed 1220.61 (+2/2) m/z.

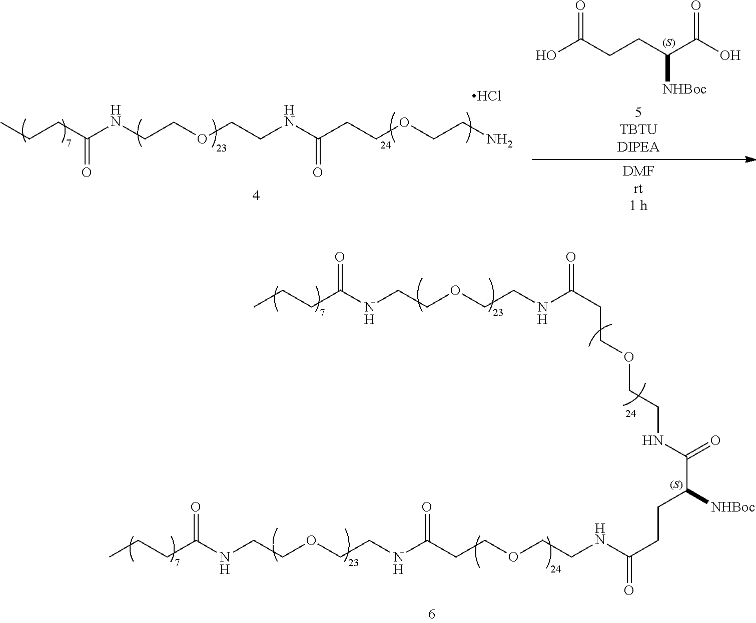

To a solution of compound 4 (253 mg), TBTU (36.1 mg), and DIEA (0.045 mL) in DMF was added compound 5 (11.9 mg) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (10-30, 35, then 100%) over 30 minutes, in which compound 6 eluted at 35% B. Compound 6 was concentrated under vacuum to provide a white oily residue. LC-MS: calculated [M+H]+ 5089.22 m/z, observed 1715.43 (+3/3, +H₂O m/z.

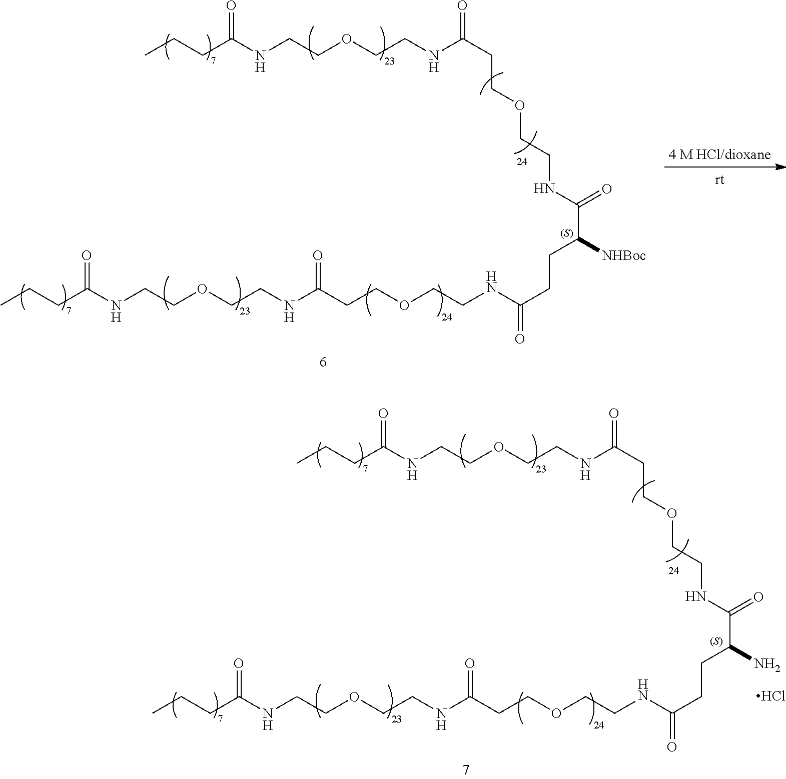

To compound 6 (28.2 mg) was added 4 M HCl/dioxane (2.0 mg) at room temperature. The reaction mixture was stirred at room temperature. The reaction mixture was stirred overnight until full conversion was confirmed via LC-MS. The reaction mixture was azeotroped with PhMe/MeOH and concentrated under high-vacuum overnight to provide an oil. LC-MS: calculated [M+H]+ 4989.17 m/z, observed 1000.21 (+5/5) m/z.

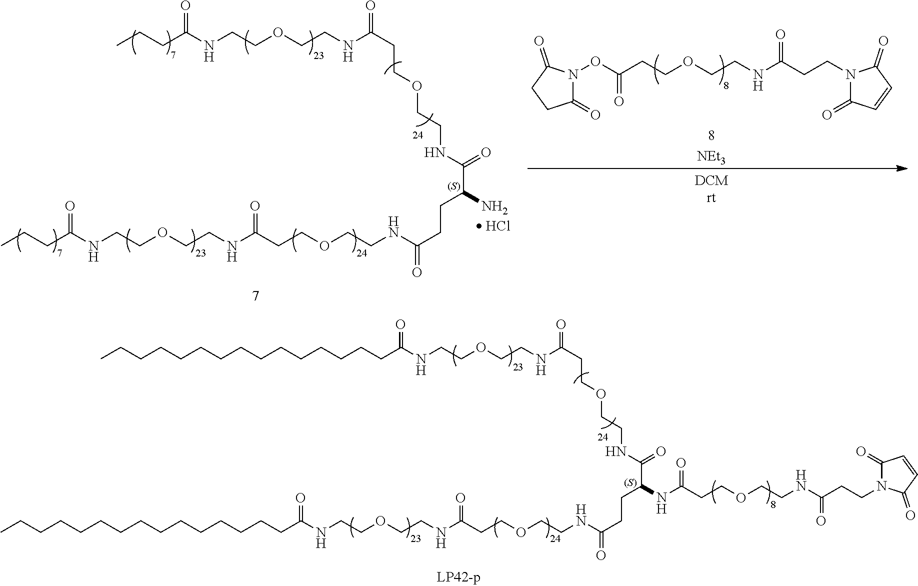

A solution of compound 7 (27.9 mg) and NEt₃ (0.004 mL) in anhydrous DCM under sparging N₂(g) was prepared at room temperature. Compound 8 (3.4 mg) was then added slowly. The reaction mixture was allowed to stir until full conversion was observed by LC-MS. The reaction mixture was then directly concentrated. The residue was purified by CombiFlash® using silica gel as the stationary phase with a gradient of 0-20% MeOH in DCM (25 to 50%, then 100% B) over 30 minutes, in which LP42-p eluted at 100% B after 5 min. at 100% B. LC-MS: calculated [M+H]+ 5563.44 m/z, observed 946.45 (+6/6, +water) m/z.

### Synthesis of LP43-p

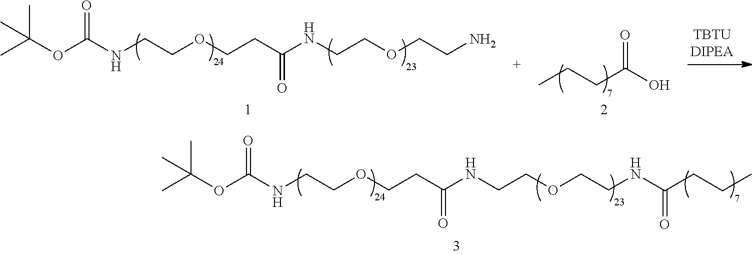

To a solution of compound 1 (3.0 g, 1.303 mmol, 1.0 equiv.), compound 2 (0.401 g, 1.564 mmol, 1.2 equiv.), and diisopropylethylamine (0.681 mL, 3.91 mmol, 3.0 equiv.) in DMF (20 mL) was added TBTU (0.502 g, 1.564 mmol, 1.2 equiv.) at room temperature. The reaction mixture was kept at room temperature for 3 hours. The reaction mixture was concentrated. Compound 3 was purified by CombiFlash® eluting with 12-18% methanol in dichloromethane. Structure confirmed by H-NMR.

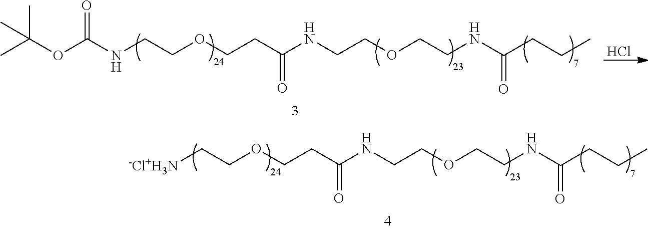

To a solid of compound 3 (2060 mg, 0.811 mmol, 1.0 equiv.) was added HCl solution in dioxane (4.055 mL, 16.219 mmol, 20 equiv.) at room temperature. The reaction mixture was kept at room temperature for 1 hour and the solvent was removed under vacuum. Compound 4 was used directly without further purification. Structure confirmed by H-NMR.

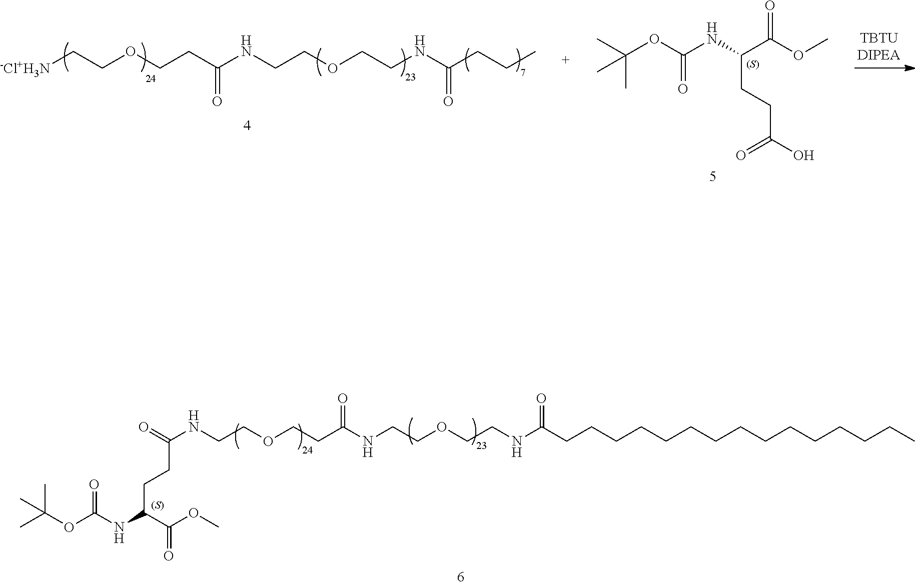

To a solution of compound 4 (2030 mg, 0.819 mmol, 1.0 equiv.), compound 5 (257 mg, 0.983 mmol, 1.2 equiv.), and diisopropylethylamine (0.428 mL, 2.459 mmol, 3.0 equiv.) in anhydrous DMF (10 mL) was added TBTU (315 mg, 0.983 mmol, 1.2 equiv.) at room temperature. The reaction mixture was kept at room temperature overnight. The reaction mixture was concentrated. Compound 6 was purified by CombiFlash® eluting with 12-20% methanol in dichloromethane. LC-MS: [M+2H]/2, calculated 1341.84 found 1342.69.

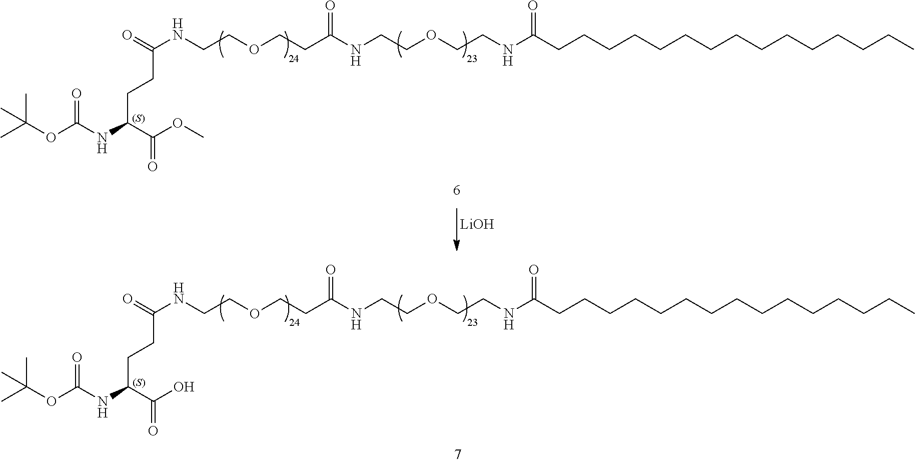

To a solution of compound 6 (1430 mg, 0.530 mmol, 1.0 equiv.) in THF (20 mL) and water (20 mL) was added lithium hydroxide (63.8 mg, 2.664 mmol, 5.0 equiv.) at room temperature. The reaction mixture was kept at room temperature for 3 hours. The reaction mixture was quenched with HCl solution and the pH was adjusted to 3.0. The aqueous phase was extracted with DCM (3×20 mL). The combined organic phases were dried over Na₂SO₄, and concentrated. Compound 7 was used directly without further purification. LC-MS: [M+2H]/2 calculated 1334.83, found 1335.49.

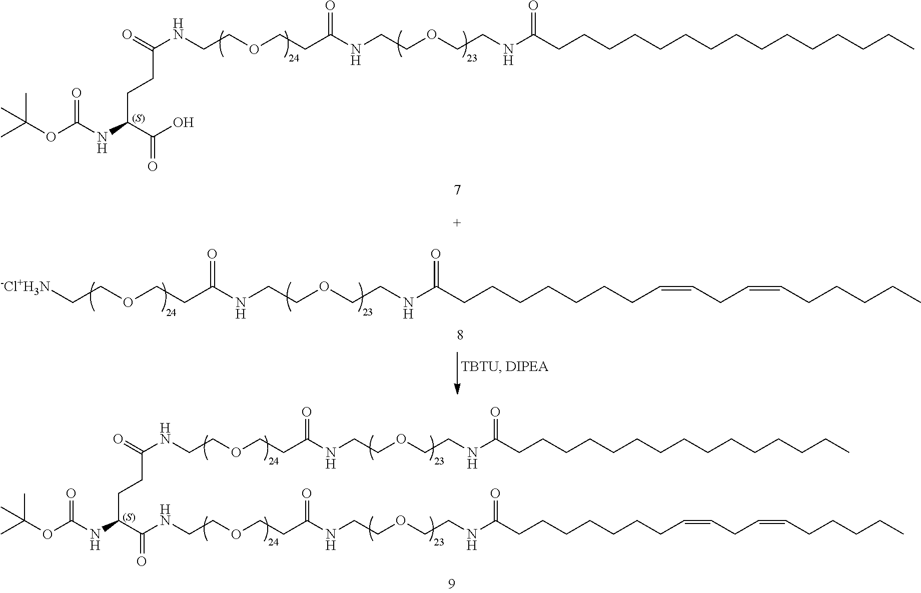

To a solution of compound 7 (110 mg, 0.0412 mmol, 1.0 equiv.), compound 8 (103 mg, 0.0412 mmol, 1.00 equiv.) and diisopropylethylamine (0.022 mL, 0.123 mmol, 3.0 equiv.) in DMF (2 mL) was added TBTU (15.9 mg, 0.0495 mmol, 1.2 equiv.) at room temperature. The reaction mixture was kept at room temperature overnight and then concentrated. Compound 9 was purified by CombiFlash® eluting with 16-20% methanol in dichloromethane. LC-MS: [M+5H]/5 calculated 1023.44, found 1024.00.

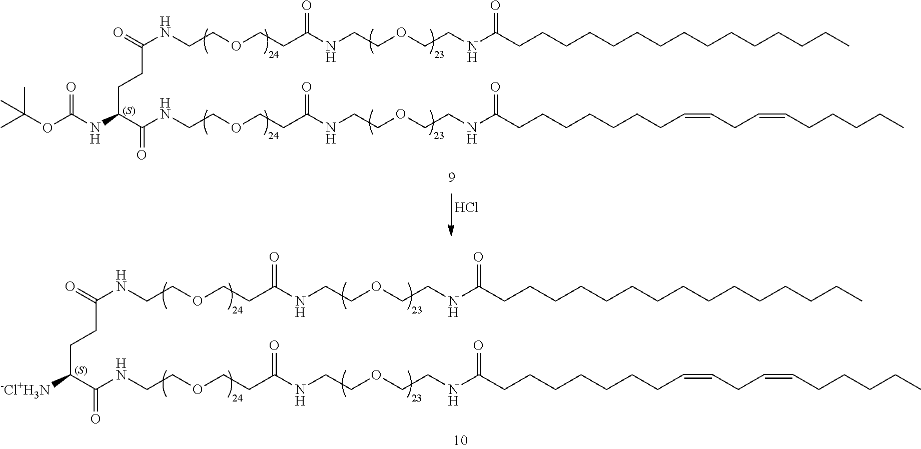

To compound 9 (84 mg, 0.0164 mmol, 1.0 equiv.) was added 4M HCl in dioxane (0.205 mL, 0.0821 mmol, 50 equiv.) at room temperature. The reaction mixture was kept at room temperature for 1 hour and then concentrated. Compound 10 was used directly without further purification. LC-MS: [M+5H]/5 calculated 1003.44, found 1004.07.

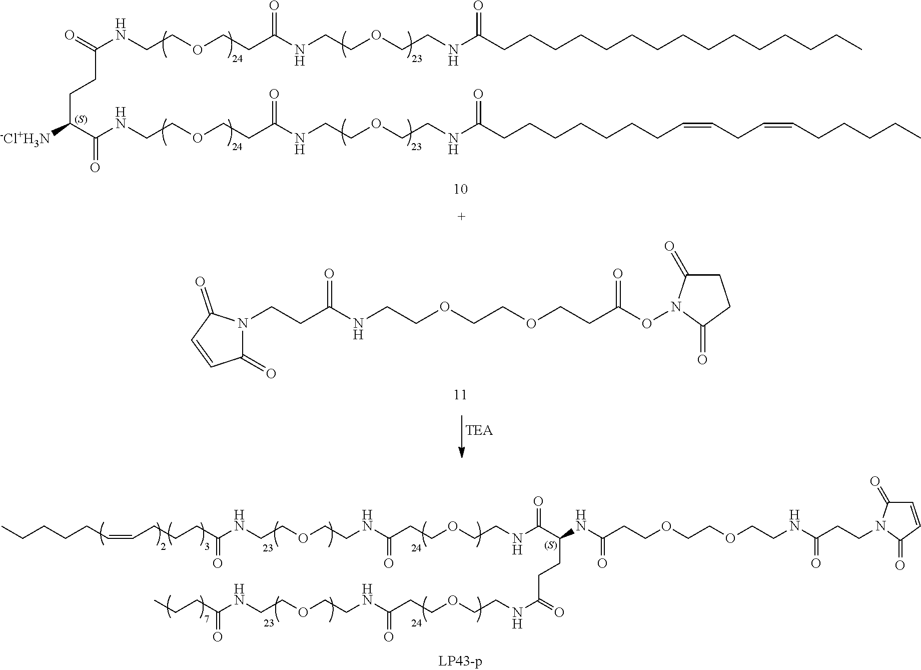

To a solution of compound 10 (125 mg, 0.0247 mmol, 1.0 equiv.) and compound 11 (116 mg, 0.0272 mmol, 1.10 equiv.) in anhydrous DCM (2 mL) was added triethylamine (0.017 mL, 0.123 mmol, 5.0 equiv.) at room temperature. The reaction mixture was kept at room temperature overnight and then concentrated. LP43-p was purified by CombiFlash® eluting with 18-20% methanol in dichloromethane. LC-MS: [M+5H]/5 calculated 1065.46, found 1066.13.

### Synthesis of LP44-p

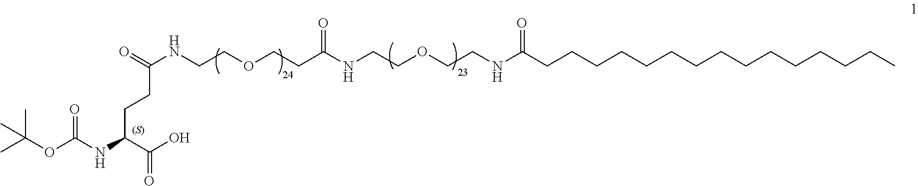

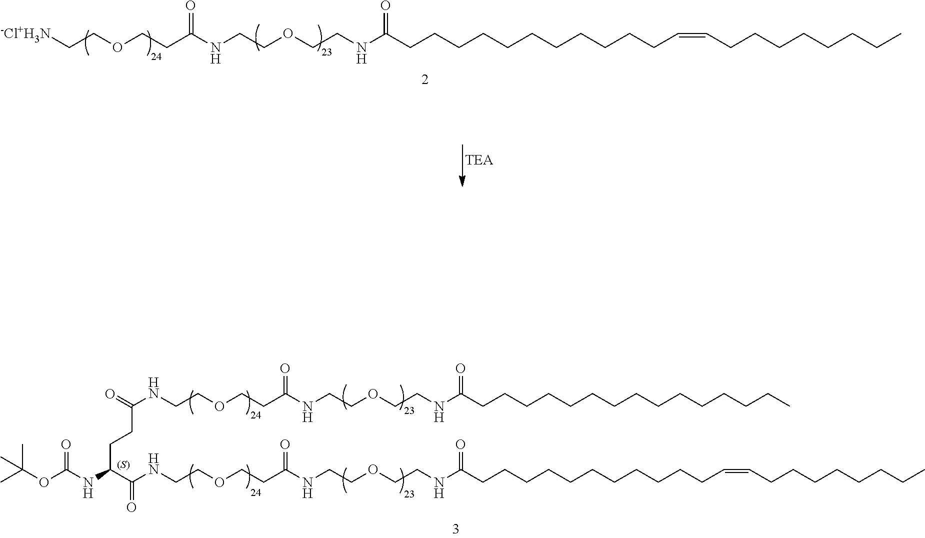

Compound 1 was synthesized as shown in the steps in the synthesis of LP43-p, above (compound 7 in synthesis of LP43-p). To a solution of compound 1 (135 mg, 0.0506 mmol, 1.0 equiv.), compound 2 (129 mg, 0.0506 mmol, 1.00 equiv.), and diisopropylethylamine (0.026 mL, 0.151 mmol, 3.0 equiv.) in DMF (2 mL) was added TBTU (19.5 mg, 0.0607 mmol, 1.2 equiv.) at room temperature. The reaction mixture was kept at room temperature overnight and then concentrated. Compound 3 was purified by CombiFlash® eluting with 12-20% methanol in dichloromethane. LC-MS: [M+5H]/5 calculated 1035.06, found 1035.40.

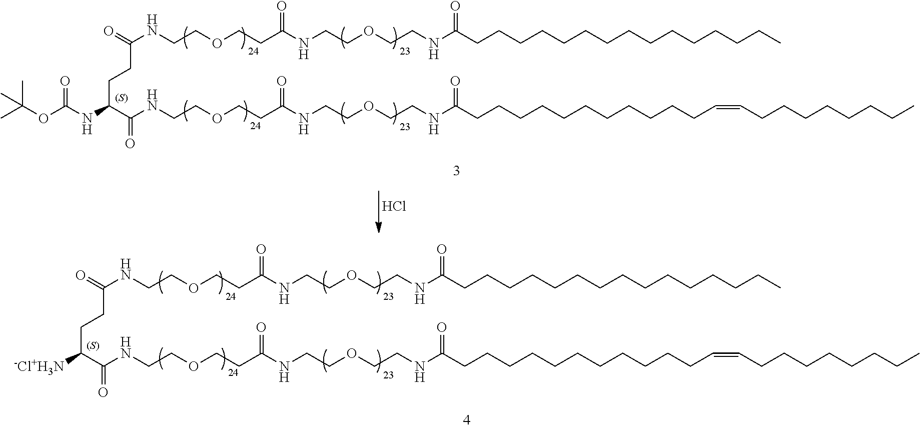

To compound 3 (100 mg, 0.0193 mmol, 1.0 equiv.) was added 4M HCl in dioxane (0.242 mL, 0.966 mmol, 50 equiv.) at room temperature. The reaction mixture was kept at room temperature for 1 hour and then concentrated. Compound 4 was used directly without further purification. LC-MS: [M+5H]/5 calculated 1015.05, found 1015.1.

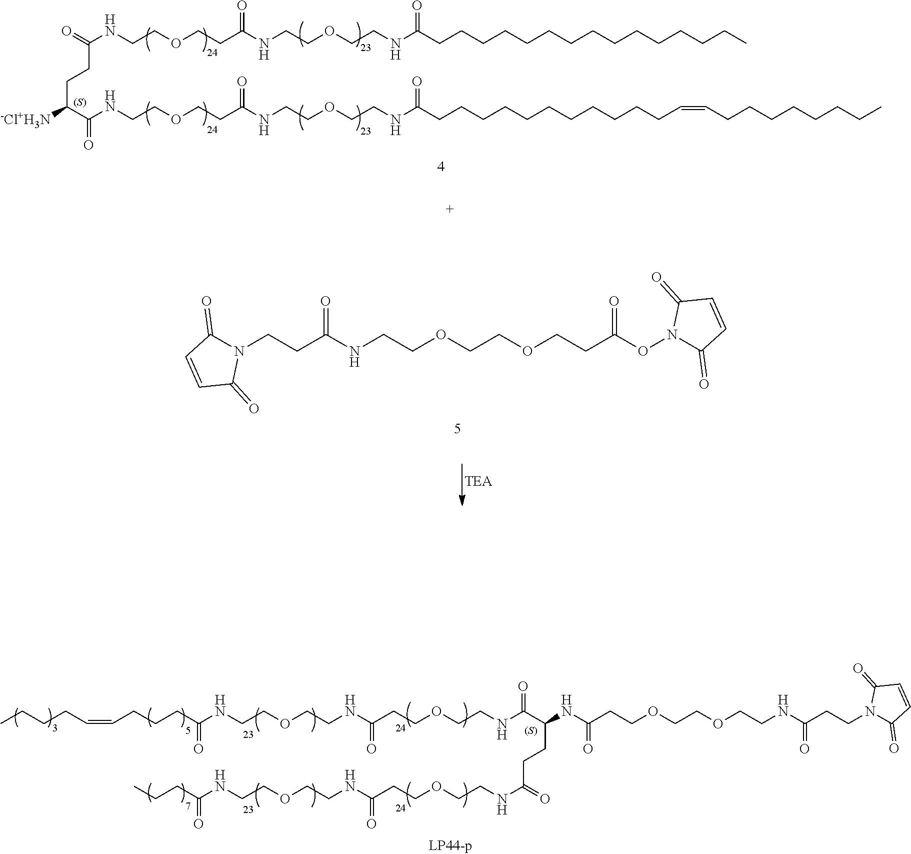

To a solution of compound 4 (95 mg, 0.0186 mmol, 1.0 equiv.) and compound 5 (8 mg, 0.0186 mmol, 1.0 equiv.) in anhydrous DCM (2 mL) was added triethylamine (0.013 mL, 0.0930 mmol, 5.0 equiv.) at room temperature. The reaction mixture was kept at room temperature overnight and then the solvent was removed under vacuum. LP44-p was purified by CombiFlash® eluting with 12-20% methanol in dichloromethane. LC-MS: calculated [M+5H]/5 1077.74, found 1079.

### Synthesis of LP45-p

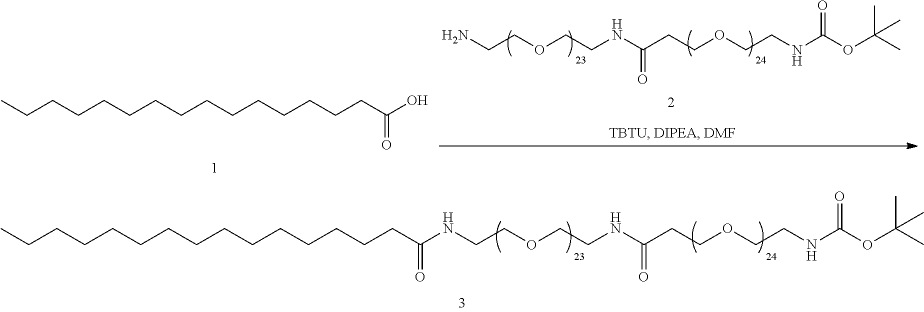

To palmitic acid 1 (30 mg, 0.1170 mmol) in a solution of DMF (2.0 mL) with Boc-PEG₄₇-NH₂ 2 (269 mg, 0.1170 mmol) was added TBTU (45.1 mg, 0.1404 mmol) and DIPEA (60 uL). After stirring the reaction mixture overnight, water was added and the compound 3 extracted using DCM:20% TFE and dried over Na₂SO₄. After filtration, the solvent was removed under vacuum to dryness and compound 3 was purified by flash chromatography (DCM:20% MeOH).

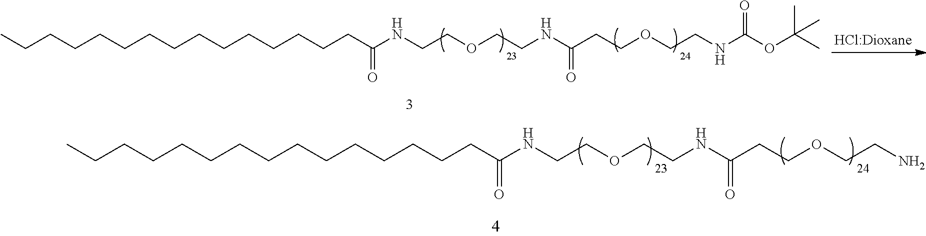

To compound 3 was added 2 mL of 4N HCl:Dioxane and the reaction mixture was stirred under anhydrous conditions until determined complete by LC-MS: calculated [M+H]+ for C₁₆-PEG₄₇-NH₂ 2301 m/z, found 2302.

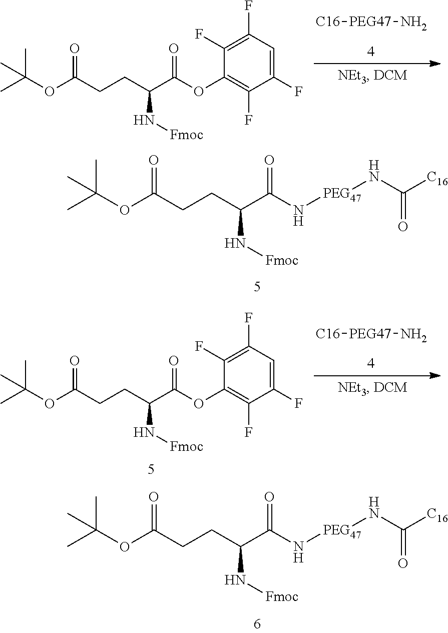

To a Fmoc-Glu(OtBu)-Opfp 5 (50 mg, 0.0845 mmol) in a solution of C₁₆-PEG₄₇-NH₂ 4 (206 mg, 0.0.0845 mmol) was added NEt₃ (29 uL), while stirring in DCM (5.0 mL). When the reaction mixture was determined complete, the solvent was removed under vacuum to dryness and the crude compound 6 was purified by flash chromatography (DCM:20% MeOH).

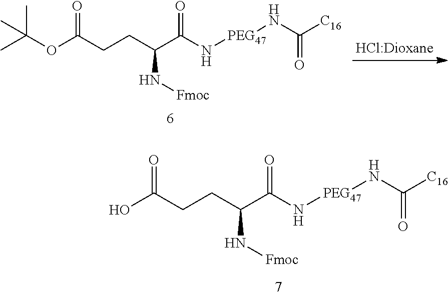

To compound 6 was added 2 mL of 4N HCl:Dioxane and stirred under anhydrous conditions until determined complete by LC-MS: Calculated 2866.0 [M+H]+ found 2867.

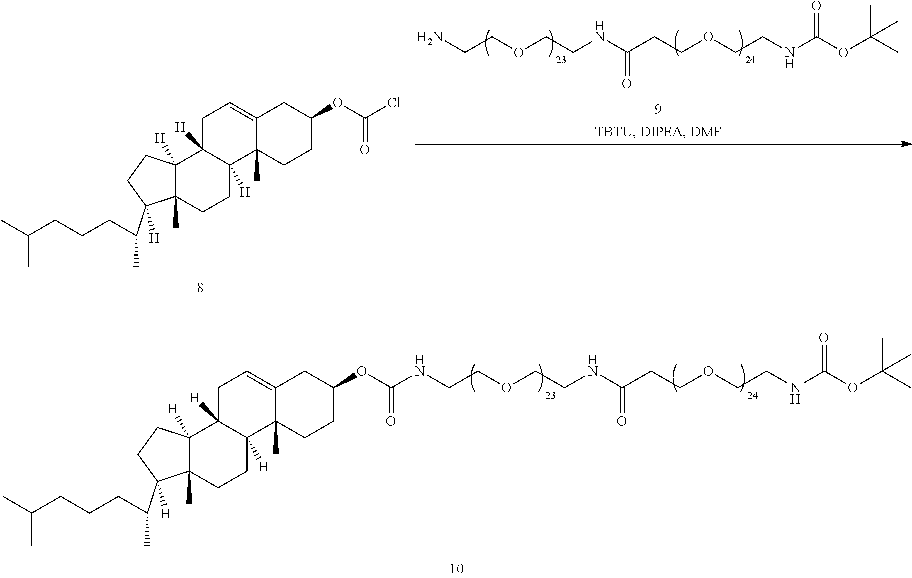

In a solution of Boc-PEG₄₇-NH₂ 9 (269 mg, 0.1170 mmol) with TBTU (45.1 mg, 0.1404 mmol) and DIPEA (60 uL), while stirring in DMF (2.0 mL) was added compound 8 (30 mg, 0.1170 mmol). After stirring the resulting suspension overnight, water was added and the product was extracted using DCM:20% TFE and dried over Na₂SO₄. After filtration, the solvent was removed under vacuum to dryness and compound 10 was purified by flash chromatography (DCM:20% MeOH). Calculated [M+H]+ for 2614.32 m/z, found 2615.32.

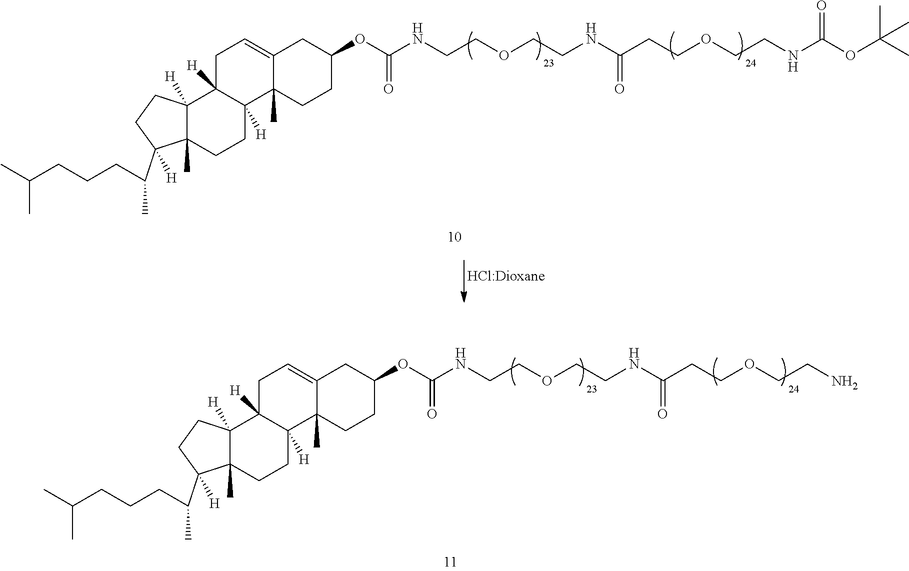

To compound 10 was added 2 mL of 4N HCl:Dioxane. The reaction mixture was stirred under anhydrous conditions until determined complete. The product 11 was used in the next step without further purification.

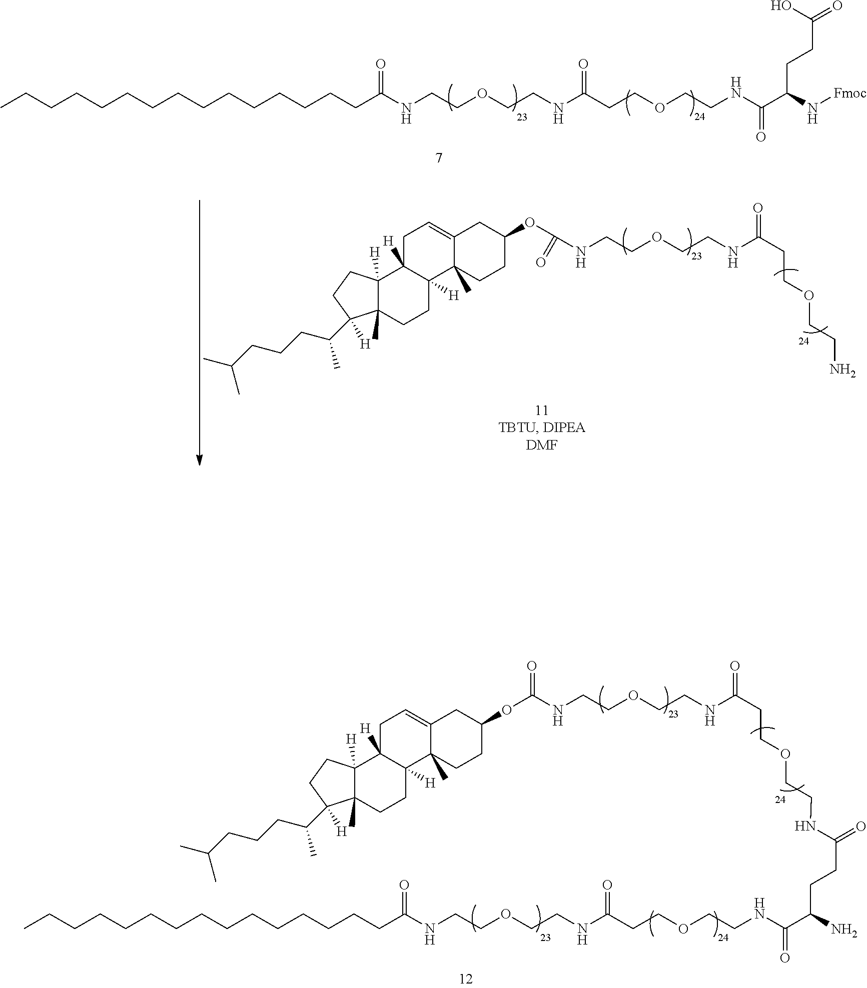

To compound 7 (100 mg, 0.0375 mmol) in a solution of DMF (5.0 mL) with compound 11 (98 mg, 0.1914 mmol) was added TBTU (14.4 mg, 0.045 mmol) and DIPEA (20 uL). After stirring the resulting suspension overnight, water was added and extracted using DCM:20% TFE and dried over Na₂SO₄. After filtration, the solvent was removed under vacuum to dryness and the purified was purified by flash chromatography (DCM:20% MeOH). To this was added 2 mL of 4N HCl:Dioxane and the reaction mixture was stirred under anhydrous conditions until determined complete by LC-MS to afford compound 12. LC-MS: calculated [M+H]+ for 5134.26 m/z, found 5135.

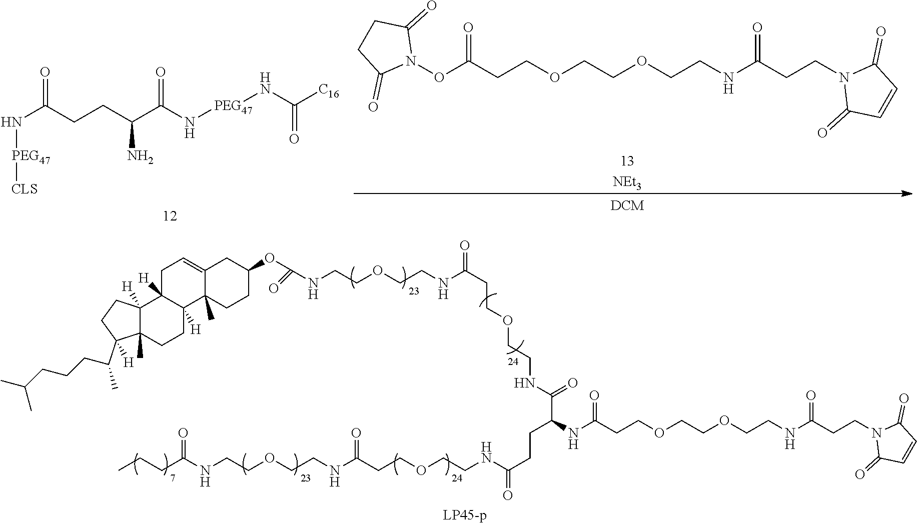

To a solution of compound 13 (10 mg, 0.0235 mmol, 1.0 equiv.) and compound 12 (120 mg, 0.0235 mmol, 1.0 equiv.) in anhydrous DCM (2 mL) was added triethylamine (17 μL, 0.1175 mmol, 5.0 equiv.) at room temperature. The reaction mixture was kept at room temperature overnight and the solvent was removed under vacuum. LP45-p was purified by CombiFlash® eluting with 10-17% methanol in dichloromethane. LC-MS: calculated [M+6H]+5474.38, found 5475.01.

### Synthesis of LP47-p

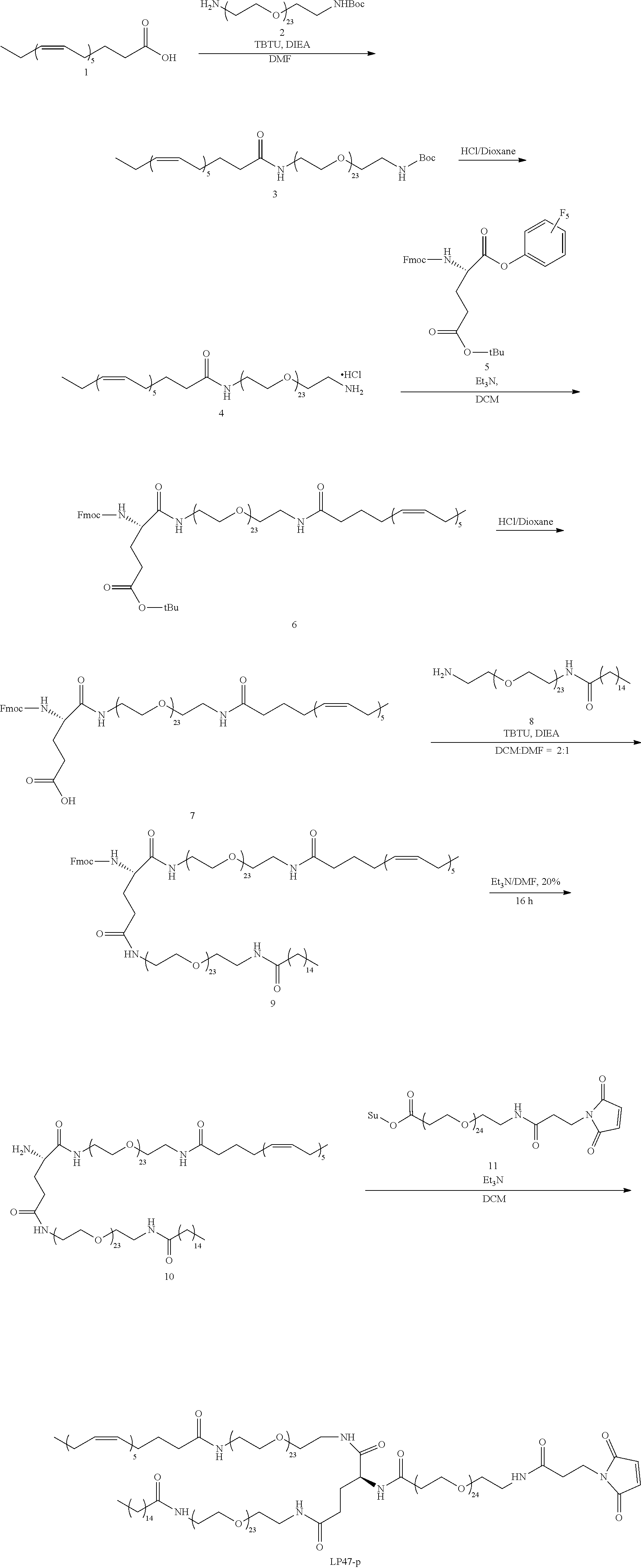

Solid TBTU (50 mg, 0.156 mmol) was added to a solution of Boc-protected Peg23-amine 2 (Quanta Biodesign Limited, 150 mg, 0.13 mmol), eicosapentaenoic acid 1 (39 mg, 0.13 mmol), and DIEA (68 μL mL, 0.39 mmol) in DMF (9 mL). The reaction mixture was sonicated to dissolve solids and stirred for 16 hours at room temperature. The solvent was removed under vacuum, toluene was evaporated twice from the residue, the residue was dissolved in chloroform (50 mL), washed with NaHCO₃ (2×10 mL) and brine (10 mL). The product was dried (Na₂SO₄), concentrated under vacuum, and purified on CombiFlash® (SiO₂) using the system 0-20% MeOH in DCM, gradient 0-80%, 20 minutes. The Boc group was removed with 4M solution of HCl in dioxane to obtain hydrochloride salt 4. Calculated MW 1357.76, (M+2)/2=679.88 Found: MS (ES, pos): 1358.29 [M+H]+, 679.77 [M+2H]2+.

Hydrochloride salt 4 (167 mg, 0.123 mmol) was stirred with pentafluorophenyl ester 5 (73 mg, 0.123 mmol) and Et₃N (43 μL, 0.31 mmol) in DCM (5 mL) for 2 hours. The solvent was removed under vacuum, the residue was mixed with SiO₂ (1 g) and loaded on CombiFlash®. The product 6 was purified using the system 0-20% MeOH in DCM, gradient 0-50%, 25 minutes. Yield 169 mg. Calculated MW 1765.23, M+18=1783.23, (M+1+18)/2=892.12 Found: MS (ES, pos): 1782.78 [M+NH4]+, 891.97 [M+H+NH4]2+.

The product 6 was treated with HCl in dioxane in order to obtain free acid 7 and directly used in the next step. Calculated MW 3002.84, (M+2×18)/2=1519.42, (M+3×18)/3=1018.95. Found: MS (ES, pos): 1519.39 [M+2NH₄]2+, 1019.17 [M+H+2NH4]3+.

The derivative 7 (47 mg, 0.028 mmol) was stirred with hydrochloride 8 (42 mg, 0.03 mmol), TBTU (11 mg, 0.034 mmol) and DIEA (18 μL, 0.1 mmol) in DCM:DMF=1:1 (8 mL) for 3 hours. The solvent was removed under vacuum, toluene was evaporated 2 times from the residue, and the solid was suspended in CHCl₃ (50 mL). The suspension was washed twice with 2% NaHCO₃ and brine. Following concentration under vacuum the product 9 was purified on CombiFlash® (0-20% MeOH in DCM, gradient 0-70%, 35 min.).

The product 9 (49 mg, 0.0162 mmol) was stirred with Et₃N in DMF (20%, 3 mL) for 16 hours, the solvent with Et₃N was removed under vacuum, toluene was evaporated 3 times from the residue to obtain deprotected amine 10, which was used directly in the next step.

Amine 10 (45 mg, 0.0162 mmol) was stirred with the mixture of NHS ester 11 (21 mg, 0.0147 mmol) and Et₃N (6 μL, 0.041 mmol) in DCM (4 mL) for 16 h. The solvent was removed under vacuum, and the product LP47-p was purified on CombiFlash® using the system DCM: 20% MeOH in DCM, gradient 0-100%, 40 min. Calculated MW 4060.07, (M+3×18)/3=1371.36, (M+4×18)/4=1033.02 Found: MS (ES, pos): 1371.76 [M+3NH₄]³⁺, 1033.70 [M+4NH₄]⁴⁺.

### Synthesis of LP48-p

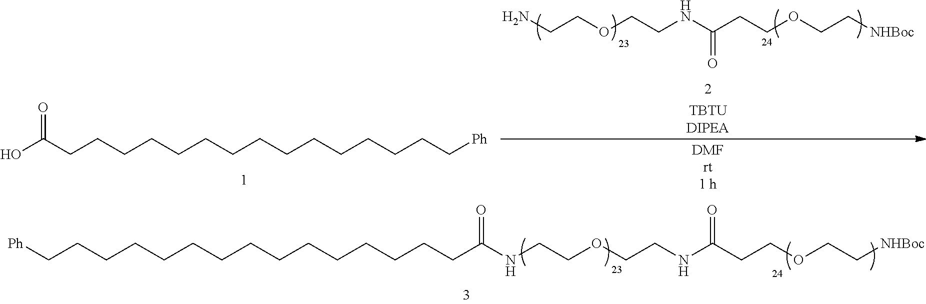

To a solution of compound 1 (27.5 mg), TBTU (26.6 mg), and DIEA (0.022 mL) in DMF was added compound 2 (173 mg) at room temperature. The reaction mixture was stirred until full conversion was observed by LC-MS. The reaction mixture was then directly concentrated. The residue was purified by CombiFlash® using a 12-g column of silica gel as the stationary phase with a gradient of 0-20% MeOH in DCM (10-100%) over 20 minutes, in which compound 3 eluted at 66% B. Compound 3 was concentrated under vacuum to provide a white oily residue. LC-MS: calculated [M+H]+ 2615.65 m/z, observed 1326.52 (+2/2, +H₂O) m/z.

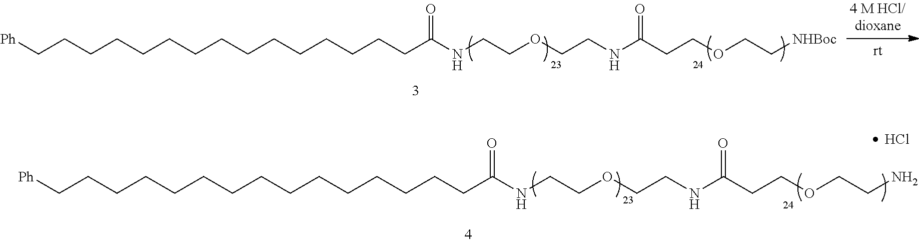

To compound 3 (56.7 mg) was added 4 M HCl/dioxane (7.9 mg) at room temperature. The reaction mixture was stirred at room temperature. Reaction was stirred overnight until full conversion was confirmed via LC-MS. The reaction mixture was azeotroped with PhMe/MeOH and concentrated under high-vacuum overnight to provide compound 4 as a white solid. LC-MS: calculated [M+H]+ 2515.60 m/z, observed 1259.91 (+2/2) m/z.

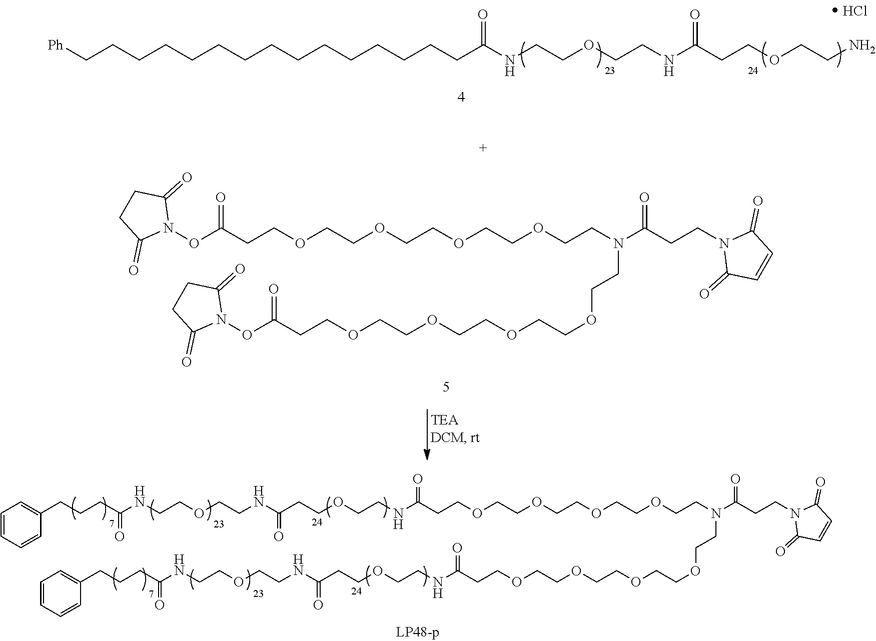

A solution of compound 4 (55.4 mg) and NEt₃ (0.015 mL) in anhydrous DCM under sparging N₂(g) was prepared at room temperature. Compound 5 (8.9 mg) was then added slowly. The reaction mixture was allowed to stir until full conversion was observed by LC-MS. The reaction mixture was then directly concentrated. The residue was purified by CombiFlash® via a 4-g column of silica gel as the stationary phase with a gradient of 0-20% MeOH/DCM (10% B to 100% B) over 20 minutes, in which LP48-p eluted at 100% B. LP48-p was concentrated to provide a white oily residue. LC-MS: calculated [M+H]+ 5558.48 m/z, observed 1152.98 (+5/5, +H₂O) m/z.

### Synthesis of LP49-p

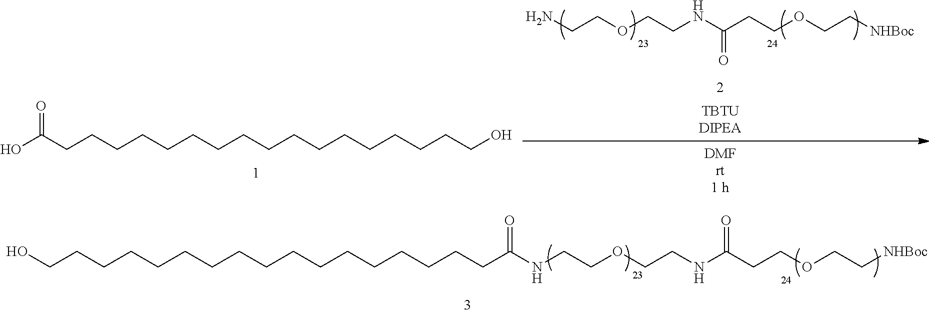

To a solution of compound 1 (31.3 mg), TBTU (33.4 mg), and DIEA (0.023 mL) in DMF was added compound 2 (199 mg) at room temperature. The reaction mixture was stirred until full conversion was observed by LC-MS. The reaction mixture was then directly concentrated. The residue was purified by CombiFlash® using silica gel as the stationary phase with a gradient of 0-20% MeOH in DCM (10-100%) over 30 minutes, in which compound 3 eluted at 57% B. Compound 3 was concentrated under vacuum to provide a white oily residue. LC-MS: calculated [M+H]+ 2583.65 m/z, observed 1311.03 (+2/2, +H₂O) m/z.

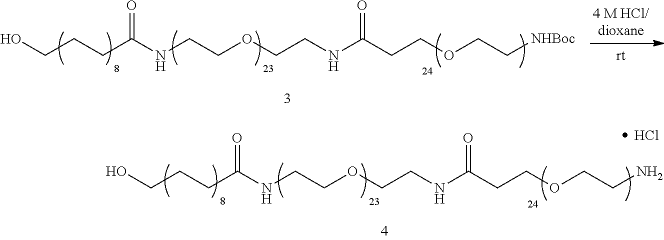

To compound 3 (70 mg) was added 4 M HCl/dioxane (9.9 mg) at room temperature. The reaction mixture was stirred at room temperature overnight until full conversion was confirmed via LC-MS. The reaction mixture was azeotroped with PhMe and concentrated under vacuum overnight to provide compound 4 as an oil. LC-MS: calculated [M+H]+ 2483.59 m/z, observed 841.32 (+2/2, +H₂O) m/z.

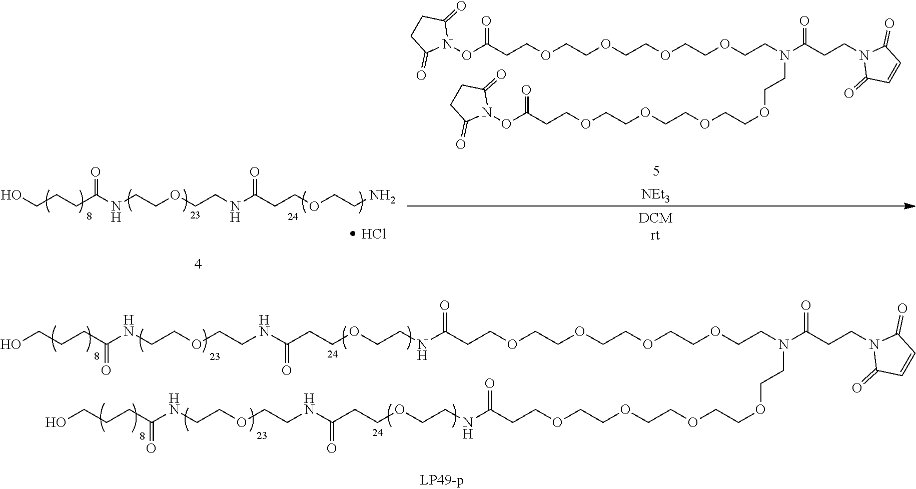

A solution of compound 4 (68.3 mg) and NEt₃ (13.7 mg) in anhydrous DCM under sparging N₂(g) was prepared at room temperature. Compound 5 (11.2 mg) was then added slowly. The reaction mixture was allowed to stir until full conversion was observed by LC-MS. The reaction mixture was then directly concentrated. The residue was purified by CombiFlash® via a 4-g column of silica gel as the stationary phase with a gradient of 0-20% MeOH in DCM (10% B to 100% B) over 20 minutes, in which LP49-p eluted at 100% B. LC-MS: calculated [M+H]+ 5594.97 m/z, observed 1418.68 (+4/4, +H₂O) m/z.

### Synthesis of LP53-p

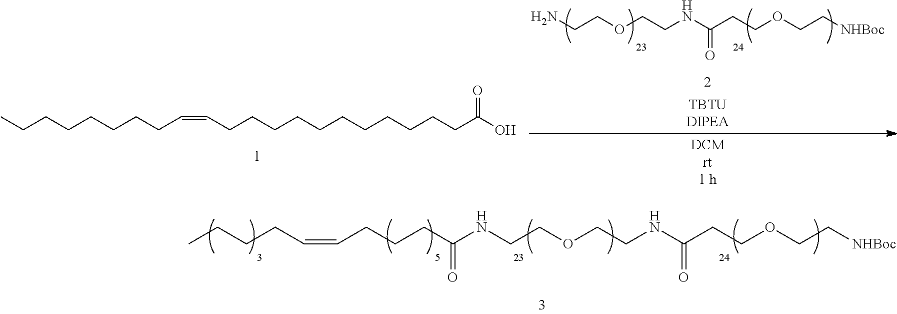

To a solution of compounds 1 (706 mg) and 2 (4.00 g) in DCM was added TBTU (670 mg) and then DIPEA (0.908 mL) at room temperature. The reaction mixture was stirred until full conversion was observed by LC-MS. The reaction mixture was then directly concentrated for isolation. The residue was purified by CombiFlash® using liquid injection with a gradient of 0-20% MeOH in DCM (0-100%) over 40 minutes. Compound 3 was concentrated under vacuum to provide a white oily residue.

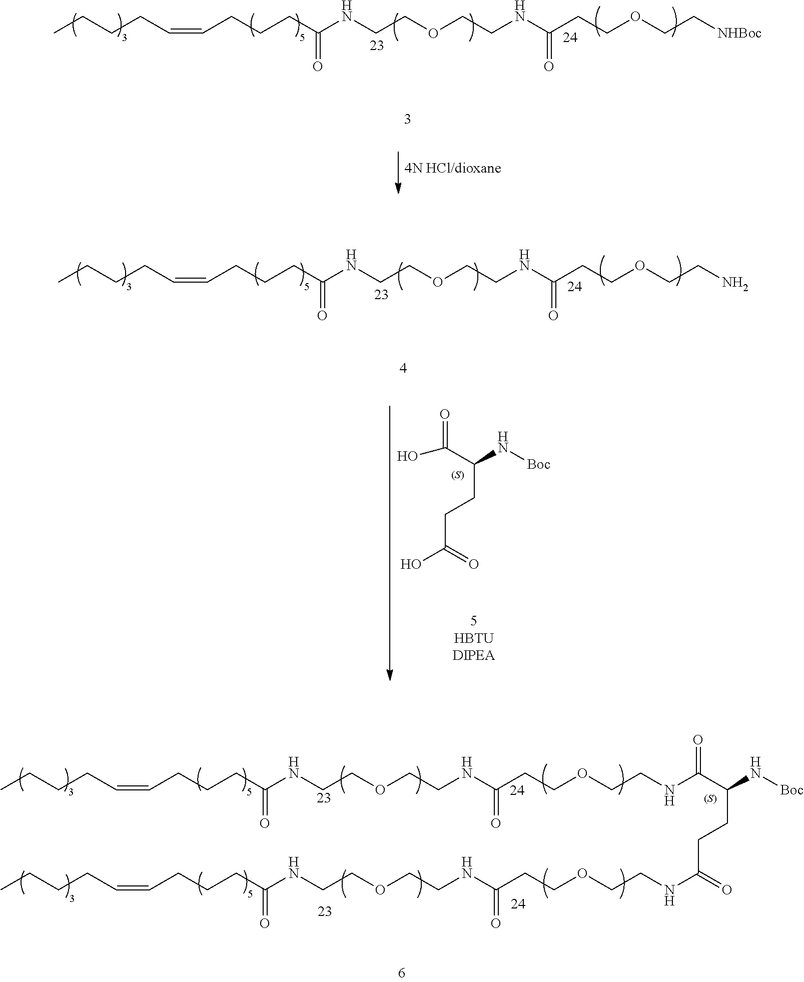

To compound 3 (4.00 g) was added 25 mL 4 M HCl/dioxane at room temperature. The reaction mixture was stirred at room temperature for 1.5 hours until full conversion was confirmed via LC-MS. The reaction mixture was then concentrated under vacuum. The residue was dissolved in DCM, then compound 5 (189 mg), HBTU (588 mg), and DIPEA (0.797 mL) were added. The reaction mixture was stirred at room temperature until full conversion was observed by LC-MS.

The reaction mixture was directly concentrated. The residue was purified by CombiFlash® using silica gel as the stationary phase with a gradient of 0-20% MeOH in DCM (0-100% B) to afford compound 6.

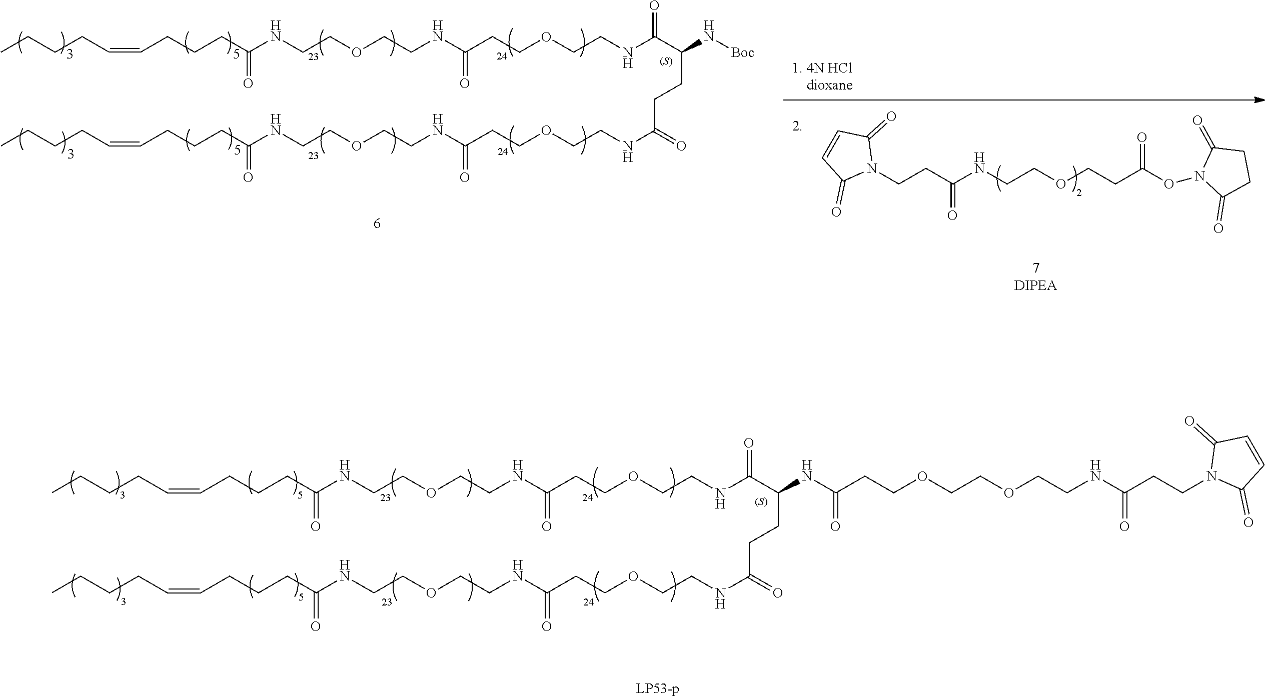

To compound 6 (2.00 g) was added 20 mL 4 M HCl/dioxane at room temperature. The reaction mixture was stirred at room temperature for 1.5 h until full conversion was confirmed via LC-MS. The reaction concentrated under vacuum. The residue was dissolved in DCM, then compound 7 (170 mg) and DIPEA (148 mg) were added. The reaction mixture was stirred at room temperature until full conversion was observed by TLC.

The product LP53-p was extracted by a standard work up (1N HCl, sat. NaHCO₃, brine). The residue was purified by CombiFlash® using silica gel as the stationary phase with a gradient of 0-20% MeOH in DCM (0-100% B).

### Synthesis of LP54-p

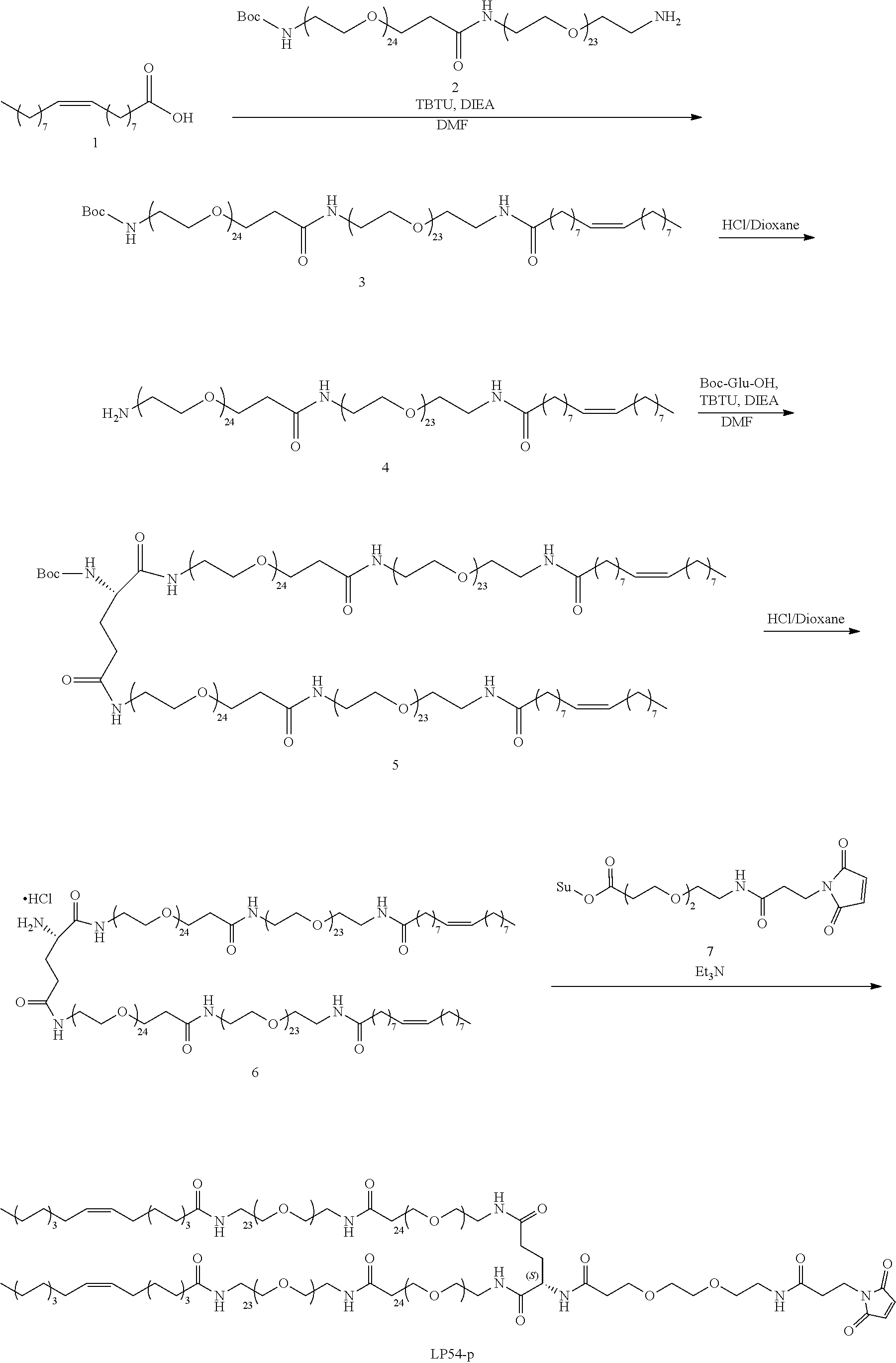

Oleic acid 1 (491 mg, 1.736 mmol) was stirred with Boc-amino-PEG₄₇ derivative 2, TBTU (670 mg, 2.086 mmol) and DIEA (908 μL, 5.21 mmol) in DMF (50 mL) for 4 h. The solvent was removed under vacuum, toluene was evaporated 3 times from the residue and the residue was suspended in CHCl₃ (150 mL). The resulting suspension was washed with H₂O, twice with 2% NaHCO₃, brine, treated with anhydrous Na₂SO₄. The mixture was concentrated to provide the product 3 which was dried under vacuum. Yield 4.391 g. Calculated MW 2566.24, (M+2×18)/2=1301.12, (M+3×18)/3=873.41 Found: MS (ES, pos): 1301.79 [M+2NH4]²⁺, 874.08 [M+3NH₄]³⁺.

Compound 3 was converted into amine hydrochloride 4 by treatment with ice-cold 4M HCl/dioxane solution (5 mL) followed by stirring at room temperature for 1 hour. The reaction mixture was concentrated and dried under vacuum, the residual HCl was removed by 2 evaporation of toluene from the product. The resulting amine hydrochloride 4 was stirred with Boc-Glu-OH (197 mg, 0.796 mmol), TBTU (594 mg, 1.85 mmol), and DIEA (1 mL, 5.74 mmol) in DMF:DCM=1:1 (60 mL) for 16 hours. The solvent was removed under vacuum, toluene was evaporated 3 times from the residue and the residue was suspended in CHCl₃ (300 mL). The suspension was washed with H₂O, twice with 2% NaHCO₃, brine, dried with anhydrous Na₂SO₄. The product 5 was purified on CombiFlash® using the system 0-20% MeOH in DCM, gradient 0-100%, 45 minutes. Yield 2.72 g. Calculated MW 5143.46, (M+3×18)/3=1732.49, (M+4×18)/4=1303.87 Found: MS (ES, pos): 1733.46 [M+3NH4]³⁺, 1304.55 [M+4NH₄]⁴⁺.

Compound 5 (2.72 g, 0.529 mmol) was stirred in 4M HCl/dioxane solution (30 mL) for 1 hour, the solvent was removed under vacuum, toluene was evaporated 2 times from the residue and the resulting dry hydrochloride salt 6 was stirred with NHS-ester 7 (212 mg, 0.5 mmol) and Et₃N in DCM (45 mL) for 16 h. The reaction mixture was diluted 3 times with CHCl₃, washed with H₂O, and brine, dried (Na₂SO₄), concentrated and product LP54-p was purified on CombiFlash® using the system 0-20% MeOH in DCM, gradient 0-100%, 55 minutes. Yield 440 mg. Calculated MW 5353.65, (M+3×18)/3=1802.55, (M+4×18)/4=1356.41 Found: MS (ES, pos): 1803.19 [M+3NH₄]³⁺, 1357.24 [M+4NH₄]⁴⁺.

### Synthesis of LP55-p

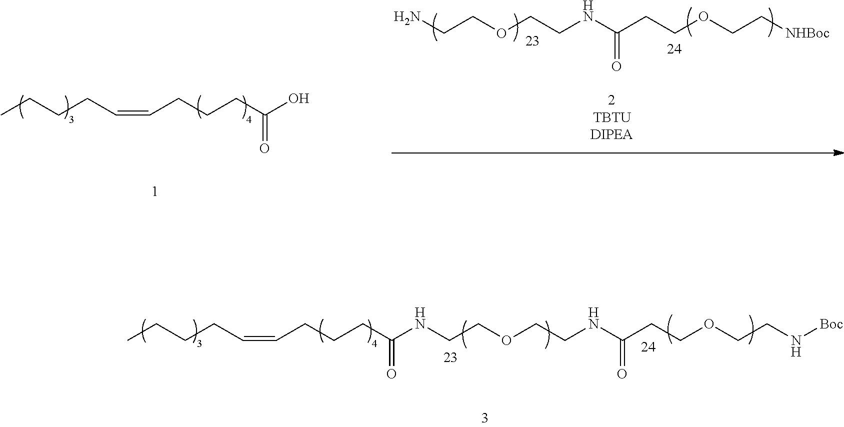

To a solution of compounds 1 (297 mg) and 2 (2.00 g) in DCM was added TBTU (307 mg) and then DIPEA (0.454 mL) at room temperature. The reaction mixture was stirred until full conversion was observed by LC-MS. The product was extracted by standard work up (1N HCl, sat. NaHCO₃, brine wash) and dried over Na₂SO₄. The crude compound 3 was used directly in the next step.

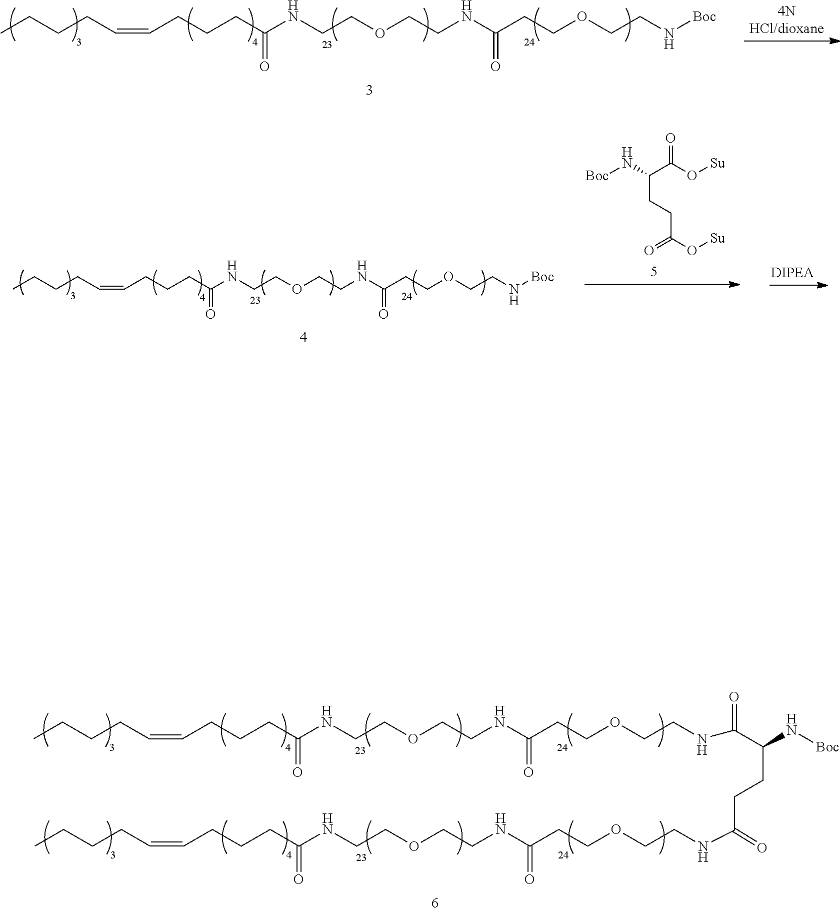

To compound 3 (2.00 g) was added 20 mL 4 M HCl/dioxane at room temperature. The reaction mixture was stirred at room temperature for 1.5 h until full conversion was confirmed via LC-MS. The reaction mixture was concentrated under vacuum. The residue was dissolved in DCM, then DIPEA (0.0403 mL) was added. followed by slow addition of compound 5 (160 mg in DCM) using a syringe pump (in 2-3 hours). The reaction mixture was stirred at room temperature until full conversion was observed by TLC.

The product was extracted using a standard work up (1N HCl, sat. NaHCO₃, brine). The residue was purified by CombiFlash® using silica gel as the stationary phase with a gradient of 0-20% MeOH in DCM (0-100% B) to afford compound 6.

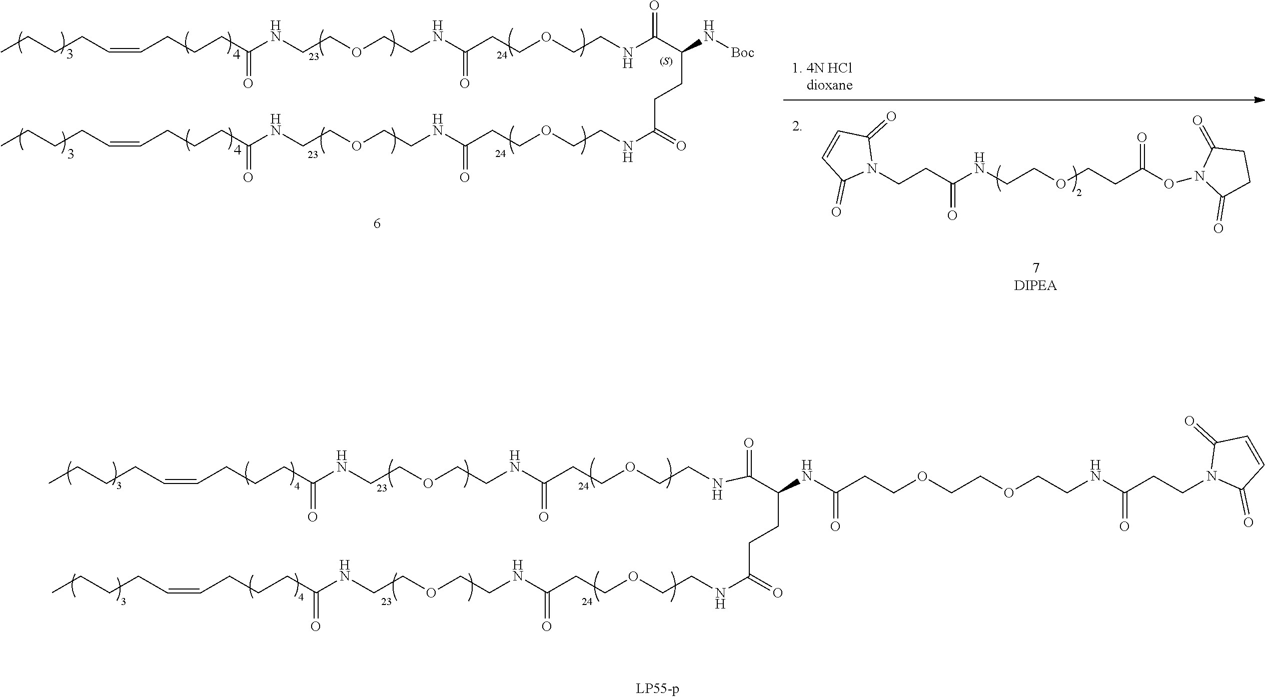

To compound 6 (1.22 g) was added 10 mL 4 M HCl/dioxane at room temperature. The reaction mixture was stirred at room temperature for 1.5 h until full conversion was confirmed via LC-MS. The reaction mixture was concentrated under vacuum. The residue was dissolved in DCM, then compound 7 (105 mg) and DIPEA (148 mg) were added. The reaction mixture was stirred at room temperature until full conversion was observed by TLC.

The product LP55-p was extracted using a standard workup (1N HCl, sat. NaHCO₃, brine). The residue was purified by CombiFlash® using silica gel as the stationary phase with a gradient of 0-20% MeOH in DCM (0-100% B).

### Synthesis of LP56-p

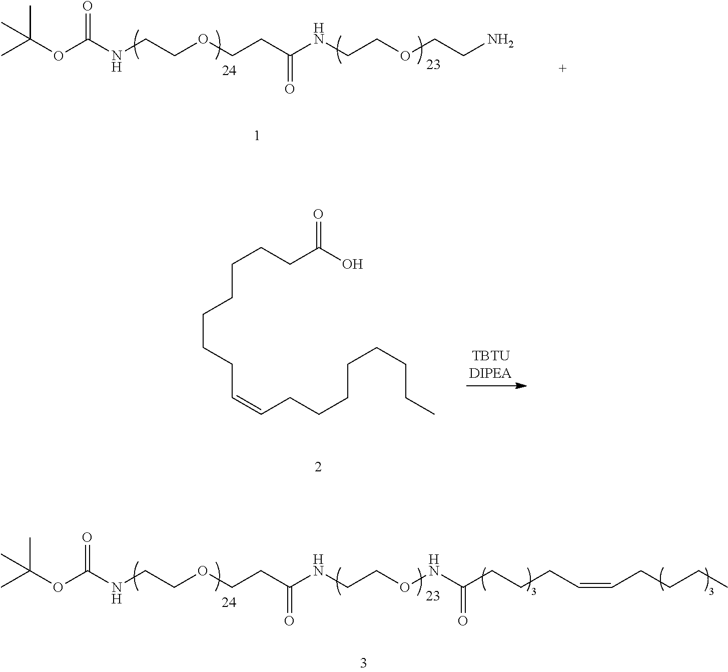

To a solution of compound 1 (150 mg, 0.0652 mmol, 1.0 equiv.), compound 2 (20 mg, 0.0717 mmol, 1.1 equiv.) and diisopropylethylamine (0.034 mL, 0.195 mmol, 3.0 equiv.) in anhydrous DMF (3 mL) was added TBTU (25.1 mg, 0.0782 mmol, 1.2 equiv.) at room temperature. The reaction mixture was kept at room temperature for 2 hours and then concentrated. Compound 3 was purified by CombiFlash® eluting with 12-18% methanol in dichloromethane. LC-MS: calculated [M+2H]+/2 1283.32, found 1283.87.

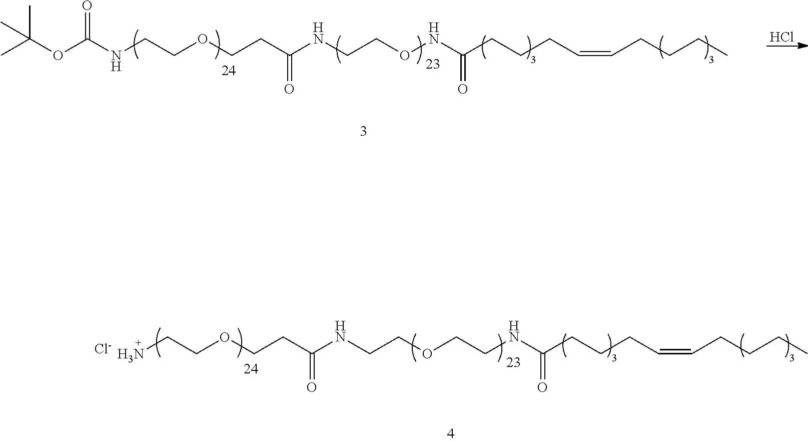

To solid of compound 3 (82 mg, 0.0320 mmol, 1.0 equiv.) was added HCl solution in dioxane (0.4 mL, 1.597 mmol, 50 equiv.) at room temperature. The reaction mixture was kept at room temperature for 30 minutes and the solvent was removed under vacuum. Compound 4 was used directly without further purification. LC-MS: calculated [M+2H]+/2 1233.29, found 1233.69.

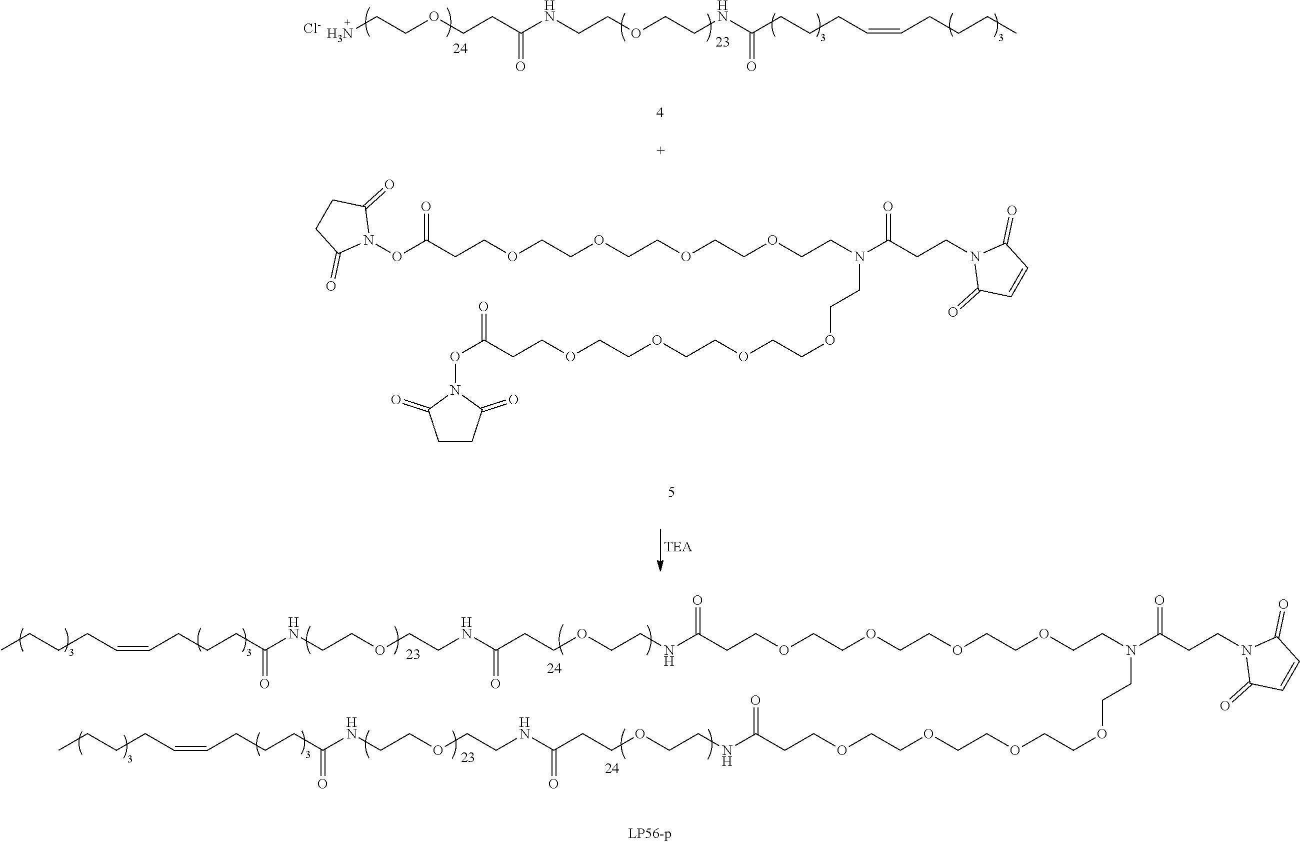

To a solution of compound 5 (13 mg, 0.0151 mmol, 1.0 equiv.) and compound 4 (77.7 mg, 0.0310 mmol, 2.05 equiv.) in anhydrous DCM (2 mL) was added triethylamine (0.011 mL, 0.0757 mmol, 5.0 equiv.) at room temperature. The reaction mixture was kept at room temperature for 1 hour and the solvent was concentrated. LP56-p was purified by CombiFlash eluting with 12-18% methanol in dichloromethane. LC-MS. calculated [M+5H]+/5 @1112.49, found 1112.34, calculated [M+6H]+/6 927.24, found 927.97.

### Synthesis of LP57-p

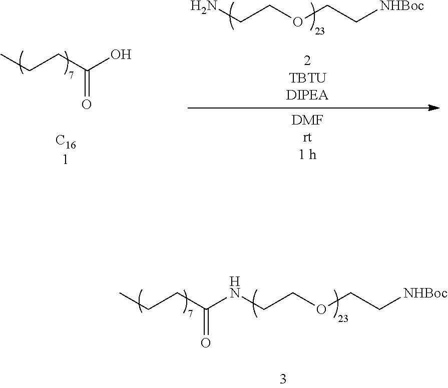

To a solution of compound 1 (787 mg), TBTU (985 mg), and DIEA (662 mg) in DMF was added compound 2 (3.06 g) at room temperature. The reaction mixture was stirred overnight until full conversion was observed by LC-MS. The reaction mixture was then washed with NaHCO₃ and extracted with 20% trifluoroethanol/DCM. The residue was purified by CombiFlash® using an 80-g column of silica gel as the stationary phase with a gradient of DCM to 20% MeOH in DCM (0-100%) over 45 min., in which compound 3 eluted at 28% B. Compound 3 was concentrated under vacuum to provide a white oily residue. LC-MS: calculated [M+H]+ 1411.95 m/z, observed 724.80 (+2/2, +H₂O) m/z.

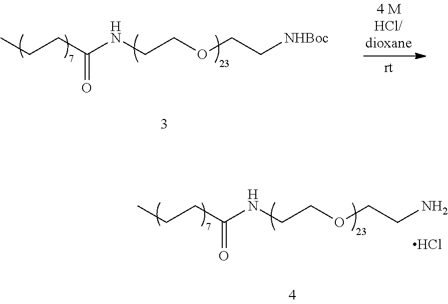

To compound 3 (1.27 g) was added 4 M HCl/dioxane (329 mg) at room temperature. The reaction mixture was stirred at room temperature until full conversion was confirmed via LC-MS. The reaction mixture was azeotroped with PhMe/MeOH and concentrated under high-vacuum overnight to provide compound 4 as a white solid. LC-MS: calculated [M+H]+ 1311.90 m/z, observed 657.59 (+2/2) m/z.

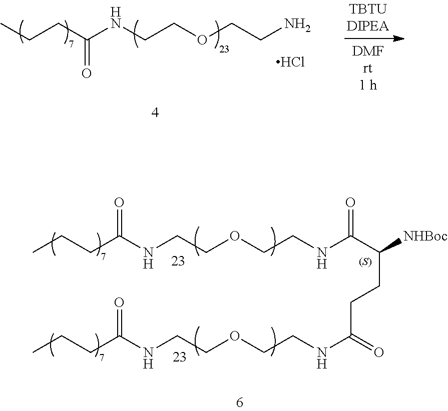

To a solution of compound 4 (1.22 g), TBTU (348 mg), and DIEA (0.3825 mL) in DMF was added compound 5 (109 mg) at room temperature. The reaction mixture was stirred until full conversion was observed by LC-MS. The reaction mixture was then washed with NaHCO₃, extracted with 20% 2, 2, 2-trifluoroethanol (TFE)/DCM, washed with NH₄Cl soln., dried over Na₂SO₄, filtered, and concentrated under vacuum. The residue was purified by CombiFlash® using silica gel as the stationary phase with a gradient of 0-20% MeOH in DCM (0-100%) over 30 minutes, in which compound 6 eluted at 51% B. Clean and impure fractions were collected and concentrated. The impure fraction was re-isolated via DCM to 20% MeOH/DCM (0-100% B), in which compound 6 eluted at 54% B and was collected and concentrated in pure fractions. Concentration under vacuum provided compound 6 as a white oily residue. LC-MS: calculated [M+H]+ 2833.89 m/z, observed 727.56 (+4/4, +H₂O) m/z.

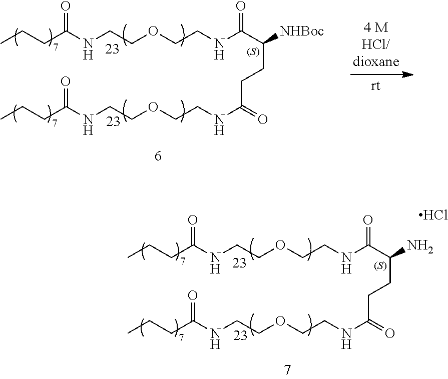

To compound 6 (130 mg) was added 4 M HCl/dioxane (16.7 mg) at room temperature. The reaction mixture was stirred at room temperature until full conversion was confirmed via LC-MS. The reaction mixture was azeotroped with PhMe/MeOH and concentrated under high-vacuum overnight to provide a compound 7 as a white solid. LC-MS: calculated [M+H]+ 2769.81 m/z, observed 694.07 (+HCl, +4/4) m/z.

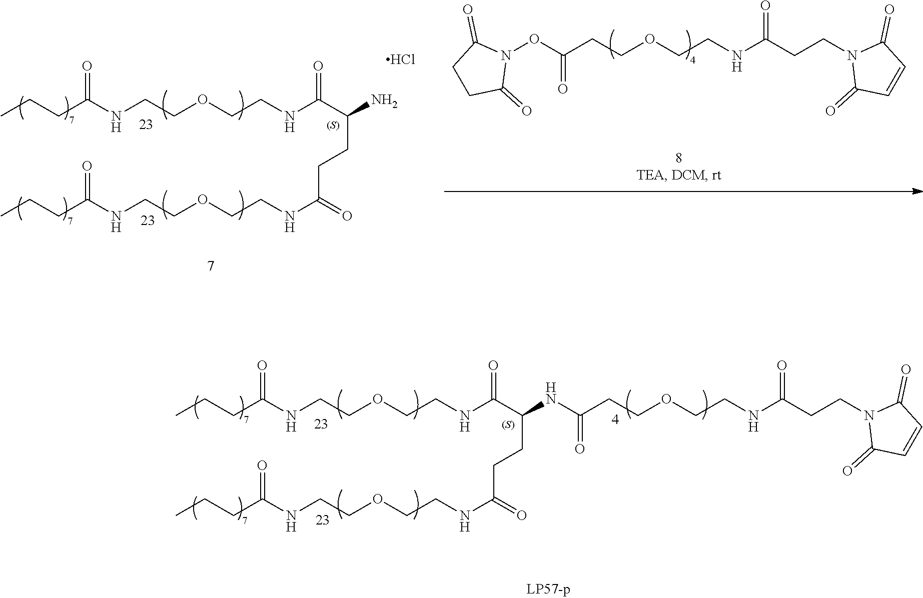

A solution of compound 7 (127 mg) and TEA (0.026 mL) in anhydrous DCM under sparging N₂(g) was prepared at room temperature. Compound 8 (24.8 mg) was then added slowly. The reaction mixture was allowed to stir until full conversion was observed by LC-MS. The reaction mixture was then directly concentrated. The residue was purified by CombiFlash® via a 12-g column of silica gel as the stationary phase with a gradient of 0-20% MeOH in DCM (0% B to 100% B) over 20 minutes, in which LP57-p eluted at 100% B. LC-MS: calculated [M+H]+ 3132.00 m/z, observed 1584.89 (+3/3, +H₂O) m/z.

### Synthesis of LP58-p

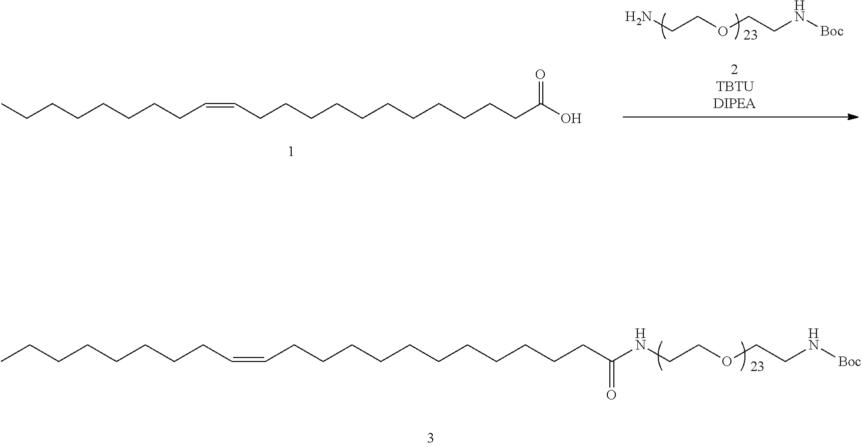

To a solution of compounds 1 (606 mg) and 2 (2.00 g) in DCM was added TBTU (657 mg) and then DIPEA (0.891 mL) at room temperature. The reaction mixture was stirred until full conversion was observed by LC-MS. The reaction mixture was then directly concentrated. The residue was purified by CombiFlash® using liquid injection with a gradient of 0-20% MeOH in DCM (0-100%) over 40 minutes. Compound 3 was concentrated under vacuum to provide a white oily residue.

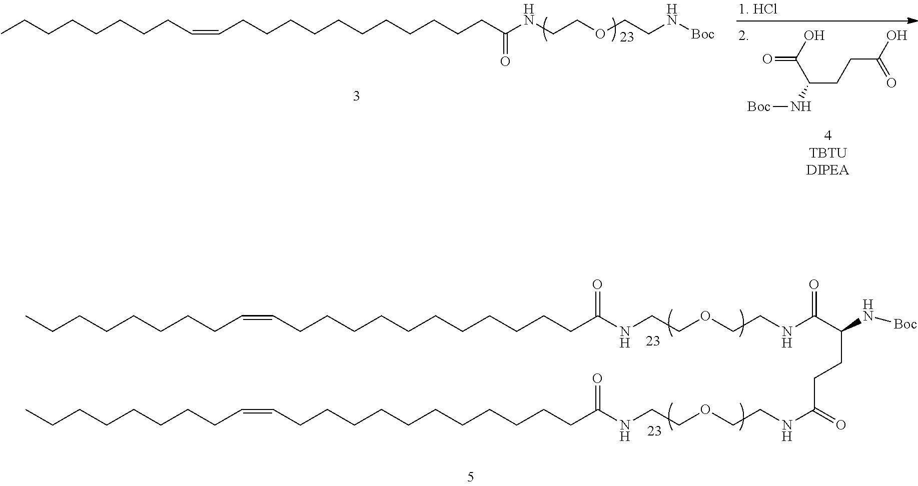

To compound 3 (2.20 g) was added 5 mL 4 M HCl/dioxane at room temperature. The reaction mixture was stirred at room temperature for 1.5 h until full conversion was confirmed via LC-MS. The reaction concentrated under vacuum. The residue was dissolved in DCM, then compound 4 (171 mg), TBTU (567 mg) and DIPEA (0.770 mL) were added. The reaction mixture was stirred at room temperature until full conversion was observed by TLC.

The product was extracted using a standard work up (1N HCl, sat. NaHCO₃, brine). The residue purified by CombiFlash® using silica gel as the stationary phase with a gradient of 0-20% MeOH in DCM (0-100% B).

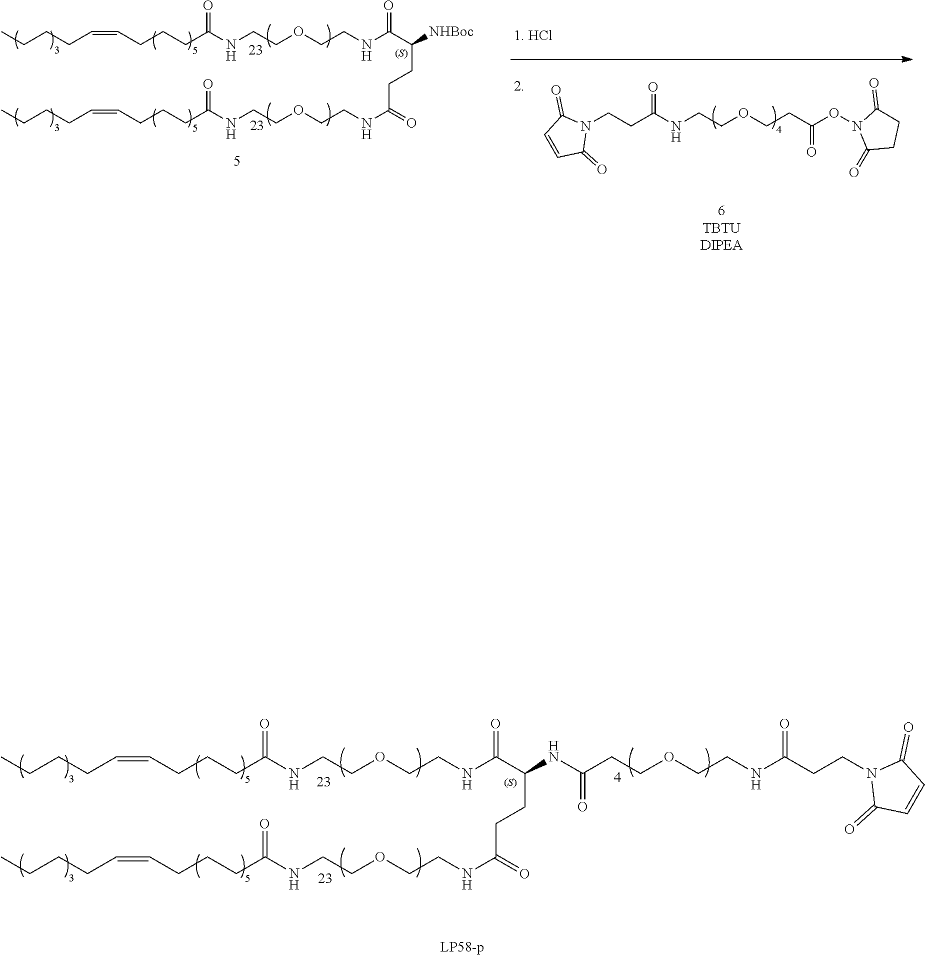

To compound 5 (1.34 g) was added 10 mL 4 M HCl/dioxane at room temperature. The reaction mixture was stirred at room temperature for 1.5 hours until full conversion was confirmed via LC-MS. The reaction concentrated under vacuum. The residue was dissolved in DCM, then compound 6, TBTU (172 mg), and DIPEA (0.234 mL) were added. The reaction mixture was stirred at room temperature until full conversion was observed by TLC.

The product LP58-p was extracted using a standard work up (1N HCl, sat. NaHCO₃, brine). The residue was purified by CombiFlash® using silica gel as the stationary phase with a gradient of 0-20% MeOH in DCM (0-100% B).

### Synthesis of LP59-p

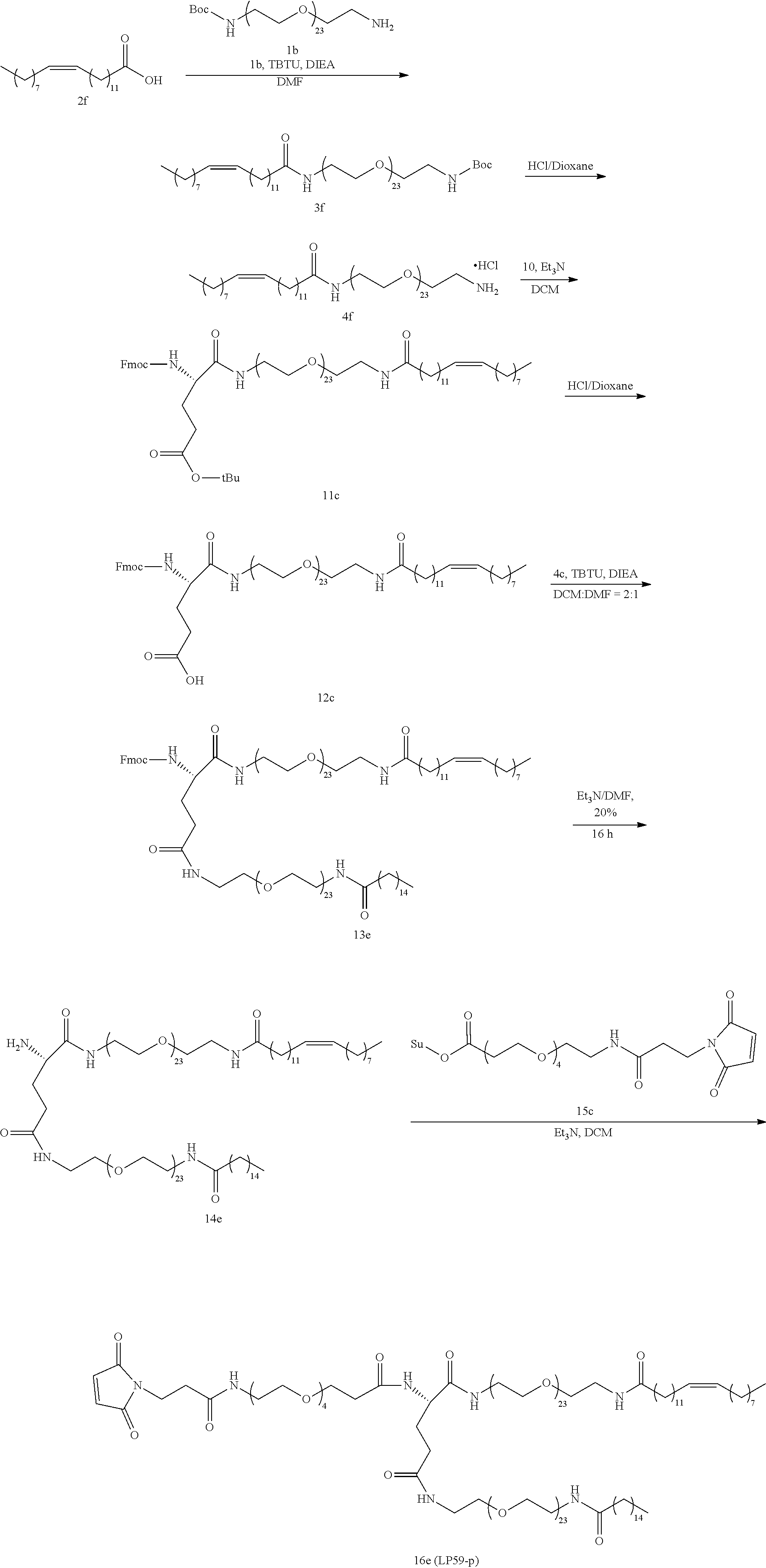

Erucic acid 2f (587 mg, 1.736 mmol) was stirred with Boc-aminopeg47 derivative 1b, TBTU (670 mg, 2.086 mmol) and DIEA (908 μL, 5.21 mmol) in DMF (50 mL) for 4 h. The solvent was removed under vacuum, toluene was evaporated 3 times from the residue, and the residue was suspended in CHCl₃ (150 mL). The resulting suspension was washed with H₂O, twice with 2% NaHCO₃, brine, and treated with anhydrous Na₂SO₄. Product 3f was isolated, concentrated and dried under vacuum. Yield 4.391 g. Calculated MW 1494.00, M+18=1512.00, (M+2×18)/2=765.00. Found: MS (ES, pos): 1512.53 [M+NH₄]+, 765.72 [M+2NH₄]²⁺.

The Boc protecting-group was removed with 4M solution of HCl in dioxane to obtain hydrochloride salt 4f (1.192 g, 0.834 mmol), which was directly used in the next step without purification. Pentafluorophenyl ester 10 (493 mg, 0.834 mmol) and Et₃N (290 μL, 2.084 mmol) in DCM (30 mL) were mixed with hydrochloride salt 4f. After 2 h of stirring the reaction mixture was diluted with CHCl₃ (150 mL), washed with H₂O, aqueous 3% NaHCO₃, and brine. The dried product 11c 1.539 g was directly used in the following step.

Compound 11c (1.539 g, 0.834 mmol) was stirred in 4M HCl/Dioxane solution (20 mL) for 4 h. The solvent was removed under vacuum, toluene was evaporated 2 times from the residue to obtain dry deprotected acid 12c (1.52 g, 0.827 mmol). This acid was stirred with amine hydrochloride 4c (1.114 g, 0.827 mmol, synthesized as shown in synthesis for LP39, above), TBTU (318.6 mg, 0.992 mmol), and DIEA (532 μL, 3.05 mmol) in a mixture of DCM:DMF=1:2 (30 mL) for 16 h. The solvent was removed under vacuum, the residual DMF was removed with 3 additional evaporations of toluene. The residue was suspended in CHCl₃ (150 mL), washed with H₂O, twice with 3% NaHCO₃, and brine. Following drying with Na₂SO₄, the product 13e was concentrated and purified on CombiFlash® using the system DCM: 20% MeOH in DCM, gradient 0-100%, 55 min. Yield 1.429 g. Calculated MW 3038.96, (M+2×18)/2=1537.48, (M+3×18)/3=1030.99 Found: MS (ES, pos): 1537.97 [M+2NH₄]²⁺, 1031.66 [M+3NH₄]³⁺.

The product 13e was Fmoc-deprotected as described in the procedure for LP39, above. The product 14e was dried and reacted with NHS-ester 15c as described in the procedure for LP39, above. The product 16e (LP59-p) was isolated using CombiFlash® purification. Calculated MW 3215.13, (M+2×18)/2=1625.57, (M+3×18)/4=1089.71. Found: MS (ES, pos): 1626.30 [M+2NH₄]²⁺, 1090.58 [M+3NH₄]³⁺.

### Synthesis of LP60-p

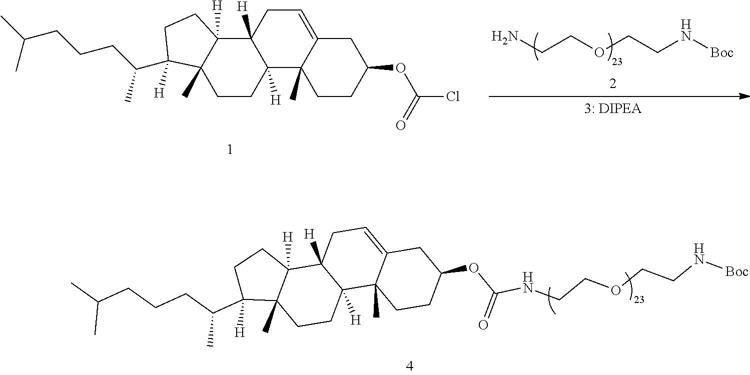

To a solution of compounds 1 (278 mg) and 2 (1.00 g) in DCM was added compound 3 (DIPEA, 0.223 mL). The reaction mixture was stirred until full conversion of 2 was observed by TLC. The product was extracted using a standard work up (1N HCl, sat. NaHCO₃, brine) and dried over Na₂SO₄. The crude compound 4 was used directly in the next step.

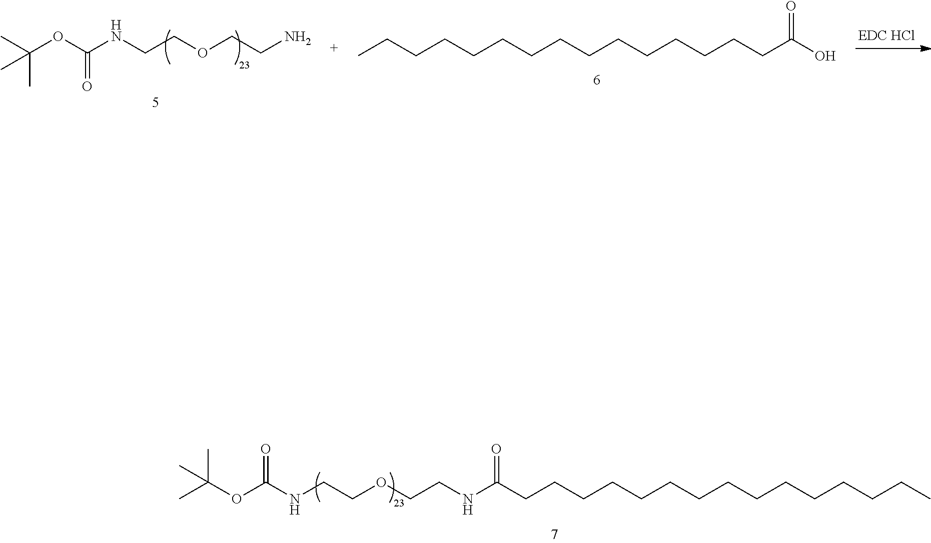

To a solution of compound 5 (2500 mg, 2.130 mmol, 1.0 equiv.) and compound 6 (655 mg, 2.556 mmol, 1.2 equiv.) in anhydrous DCM (10 mL) was added EDC HCl (630 mg, 3.195 mmol, 1.5 equiv.) at room temperature. The reaction mixture was kept at room temperature overnight. The reaction mixture was concentrated. The product was purified by CombiFlash® eluting with 12-20% methanol in dichloromethane. LC-MS: calculated [M+H]+ 1411.95, found 1413.64.

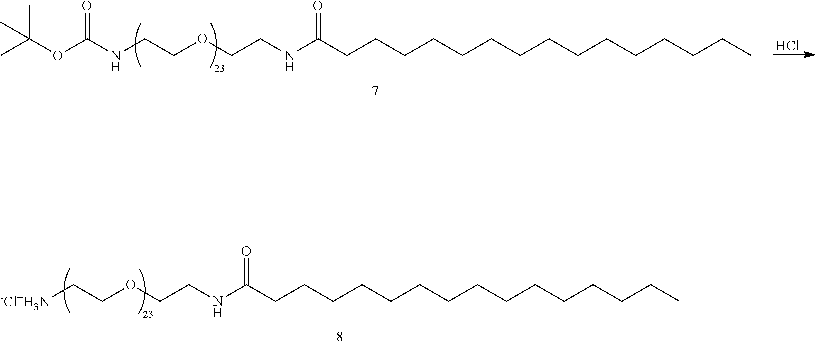

To a solid of compound 7 (2100 mg, 1.487 mmol, 1.0 equiv.) was added HCl solution in dioxane (7.438 mL, 29.75 mmol, 20 equiv.) at room temperature. The reaction mixture was kept at room temperature for 1 hour and the solvent was concentrated. Compound 8 was used directly without further purification. LC-MS: calculated [M+H]+ 1311.90, found 1312.95.

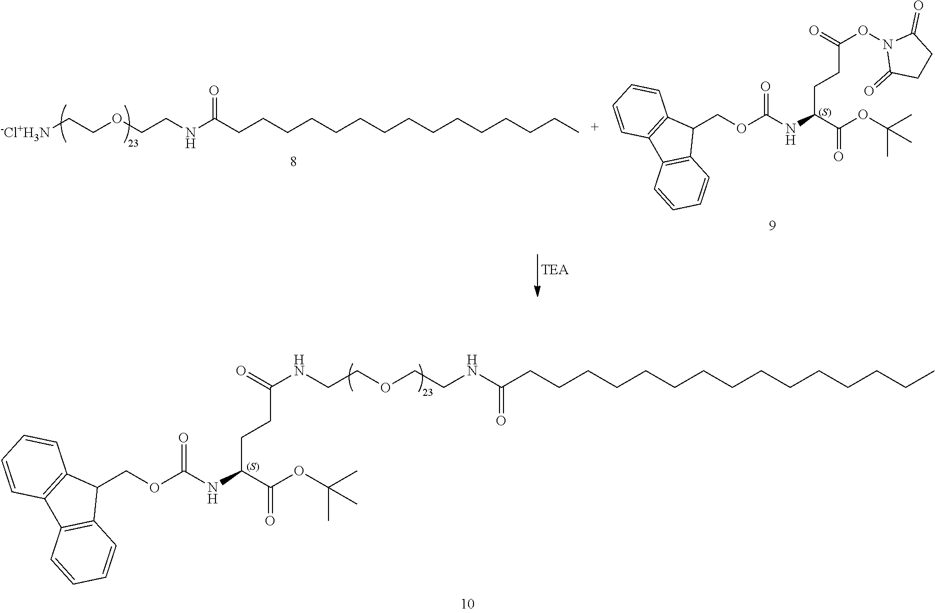

To a solution of compound 8 (1210 mg, 0.897 mmol, 1.0 equiv.) and compound 9 (539 mg, 1.032 mmol, 1.15 equiv.) in anhydrous DCM (10 mL) was added triethylamine (0.381 mL, 2.692 mmol, 3.0 equiv.) at room temperature. The reaction mixture was kept at room temperature for 2 hours. The organic phase was washed with saturated NH₄Cl and saturated NaHCO₃ aqueous solution. The organic phase was dried over Na₂SO₄ and concentrated. Compound 10 was separated by CombiFlash® and eluting with 12-20% methanol in dichloromethane. LC-MS: calculated [M+H]+ 1719.07, found 1719.42.

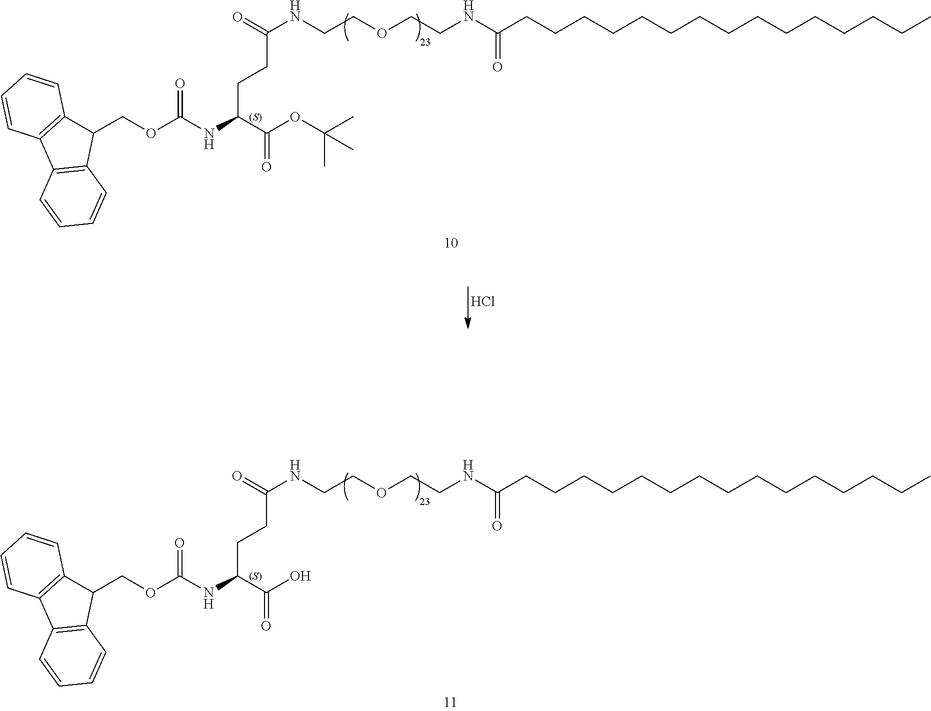

To compound 10 (1100 mg, 0.639 mmol, 1.0 equiv.) was added 4M HCl in dioxane (3.199 mL, 12.796 mmol, 20 equiv.) at room temperature. The reaction mixture was kept at room temperature for 8 hours. The reaction mixture was concentrated. Compound 11 was used directly without further purification. LC-MS: [M+H]+ calculated 1663.01, found 1664.00.

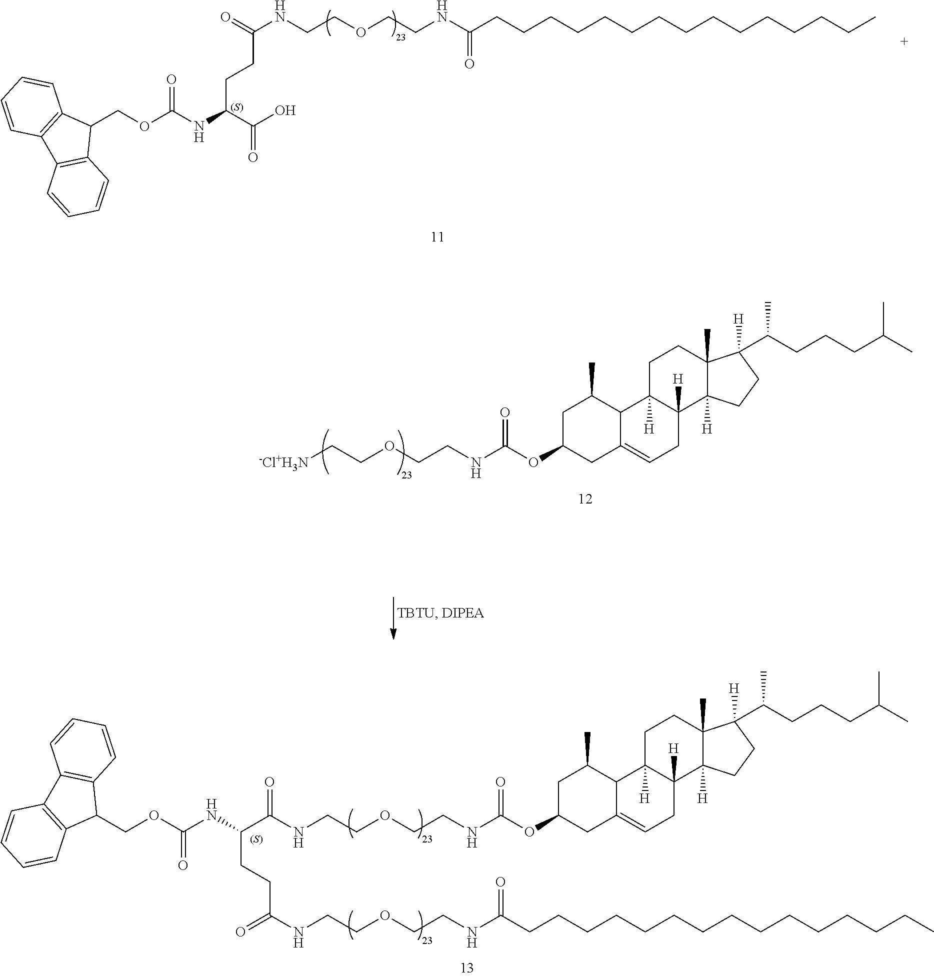

To a solution of compound 11 (1060 mg, 0.637 mmol, 1.0 equiv.), compound 12 (970 mg, 0.637 mmol, 1.00 equiv.) and diisopropylethylamine (0.444 mL, 2.549 mmol, 4.0 equiv.) in DMF (10 mL) was added TBTU (245 mg, 0.764 mmol, 1.2 equiv.) at room temperature. The reaction mixture was kept at room temperature for 2 hours. The reaction mixture was concentrated. The residue was washed with saturated ammonium chloride and sodium bicarbonate aqueous solution. Compound 13 was purified by CombiFlash® eluting with 10-20% methanol in dichloromethane. LC-MS: [M+2H]/2 calculated 1565.50, found 1567.13.

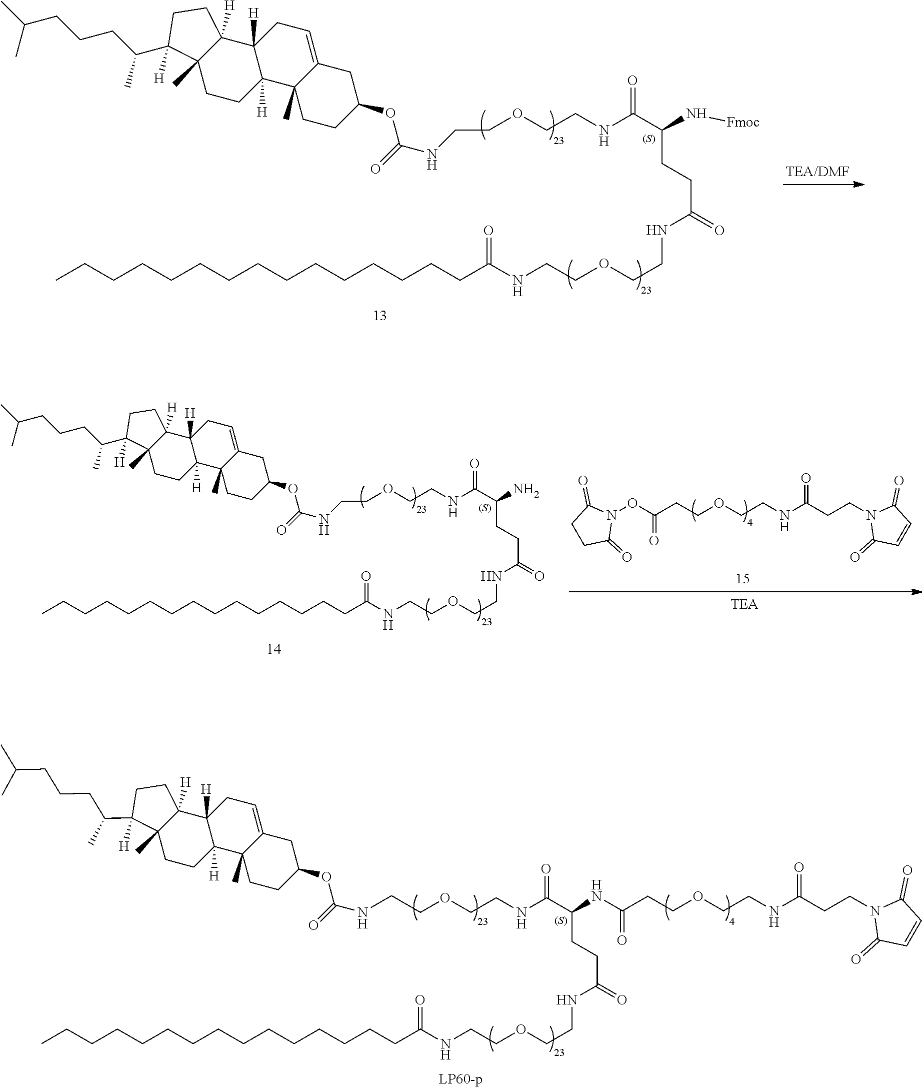

To a solution of compound 13 (1.05 g) in 4 mL of DMF was added 1 mL of TEA at room temperature. The reaction mixture was stirred overnight and the solvent was removed under vacuum to afford compound 14. Compound 14 was used without further purification.

To a solution of compound 14 (585 mg) in 6 mL DCM was added compound 15 (124 mg) and TEA (0.085 mL) at room temperature. The reaction mixture was stirred overnight. The product was extracted using a standard work up (1N HCl, sat. NaHCO₃, brine) and dried over Na₂SO₄. LP60-p was further purified with column chromatography.

### Synthesis of LP61-p

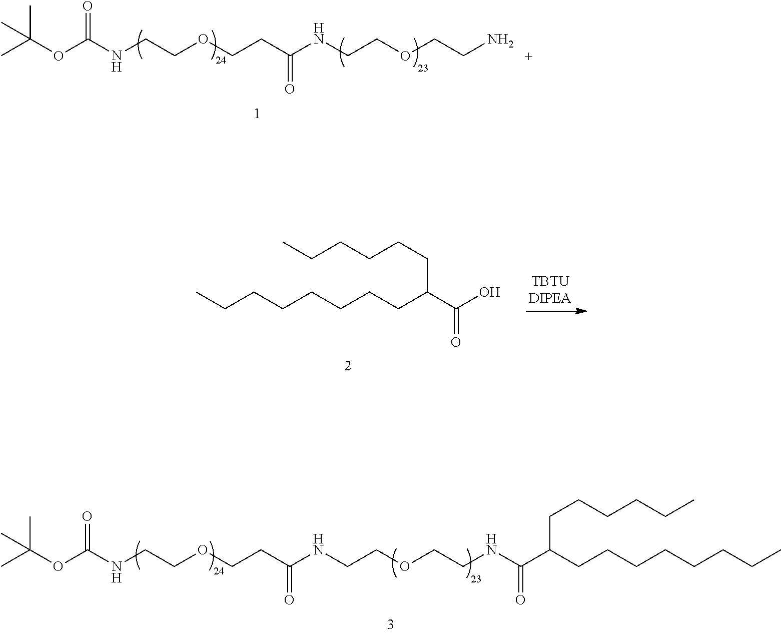

To a solution of compound 1 (124 mg, 0.0539 mmol, 1.0 equiv.), compound 2 (19.5 mg, 0.0646 mmol, 1.2 equiv.), and diisopropylethylamine (0.028 mL, 0.161 mmol, 3.0 equiv.) in anhydrous DMF (2 mL) was added TBTU (20.8 mg, 0.0646 mmol, 1.2 equiv.) at room temperature. The reaction mixture was kept at room temperature for 1 hour. The reaction mixture was quenched with saturated sodium bicarbonate aqueous solution. The aqueous phase was extracted with DCM (3×10 mL), and the combined organic phases were dried over Na₂SO₄, and concentrated. Compound 3 was purified by CombiFlash® eluting with 10-12% methanol in dichloromethane. LC-MS: calculated [M+2H]+/2 1270.31, found 1269.15.

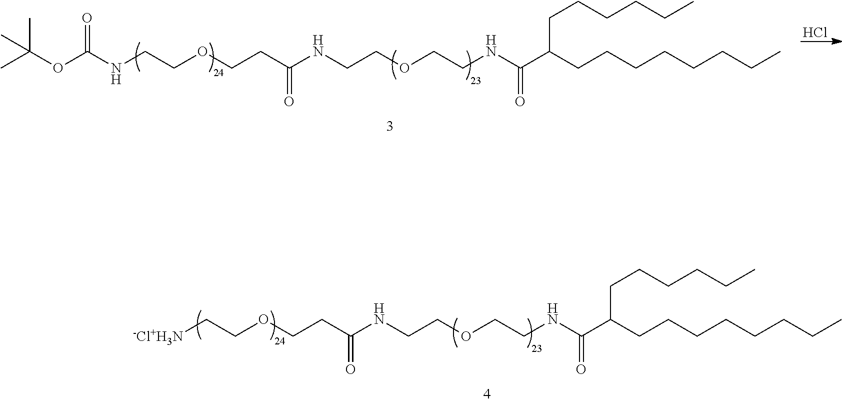

To compound 3 (56 mg, 0.0220 mmol, 1.0 equiv.) was added 4M HCl in dioxane (0.276 mL, 1.102 mmol, 50 equiv.) at room temperature. The reaction mixture was kept at room temperature for 1 hour and then concentrated. Compound 4 was used directly without further purification. LC-MS: [M+2H]/2 calculated 1220.28, found 1221.63.

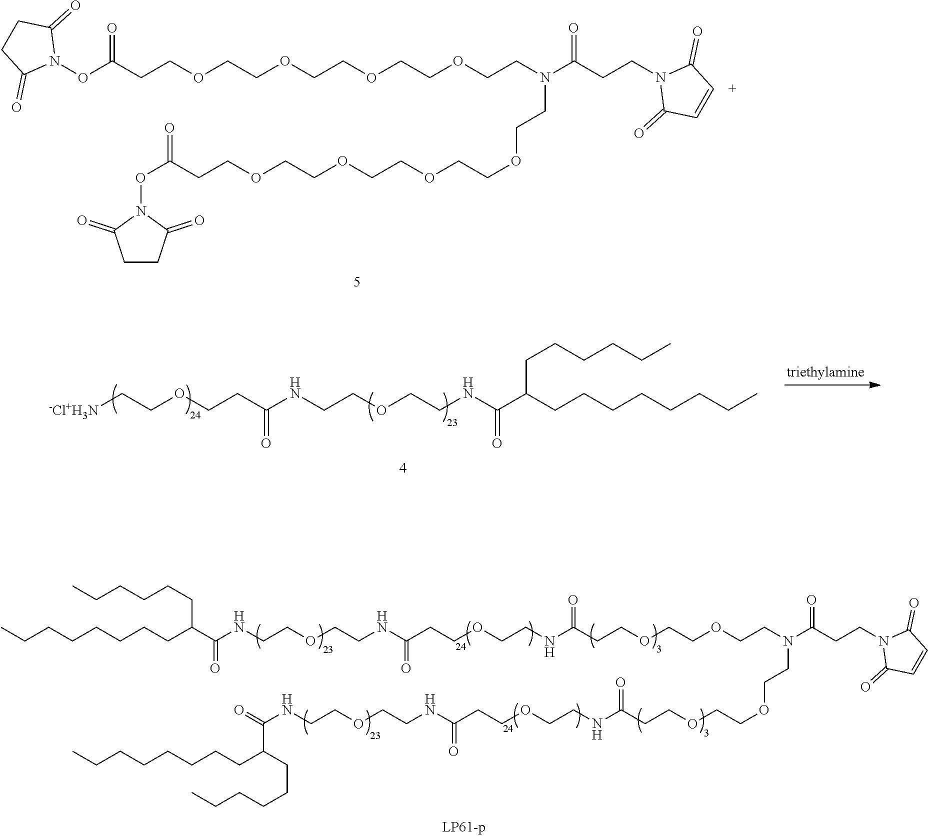

To a solution of compound 5 (10 mg, 0.0116 mmol, 1.0 equiv.) and compound 6 (59.1 mg, 0.0239 mmol, 2.05 equiv.) in anhydrous DCM (2 mL) was added triethylamine (0.008 mL, 0.0931 mmol, 5.0 equiv.) at room temperature. The reaction mixture was kept at room temperature for 4 hours and the solvent was removed under vacuum. LP61-p was purified by CombiFlash® eluting with 12-15% methanol in dichloromethane. LC-MS: calculated [M+6H]+/6 918.57, found 919.69.

### Synthesis of LP62-p

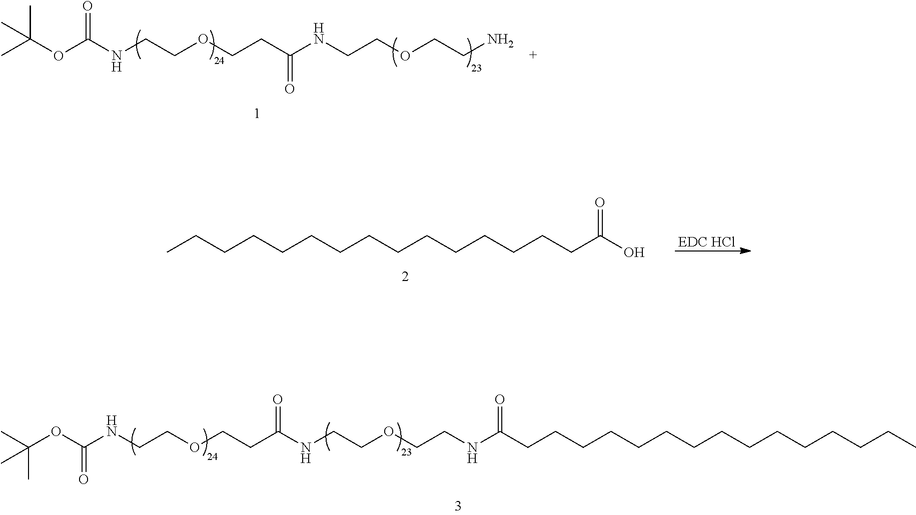

To a solution of compound 1 (1500 mg, 0.6517 mmol, 1.0 equiv.) and compound 2 (200 mg, 0.782 mmol, 1.2 equiv.) in anhydrous DCM (10 mL) was added EDC HCl (192 mg, 0.997 mmol, 1.5 equiv.) at room temperature. The reaction mixture was kept at room temperature overnight and then concentrated. Compound 3 was purified by CombiFlash® eluting with 12-20% methanol in dichloromethane. LC-MS: calculated [M+2H]+/2 1270.31, found 1271.43.

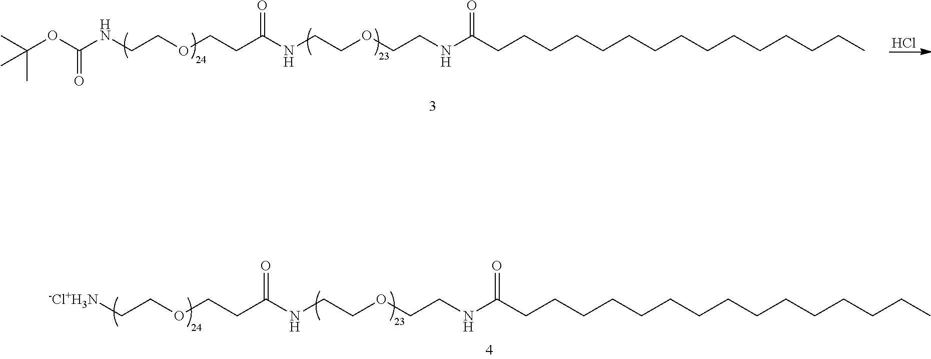

To compound 3 (1300 mg, 0.511 mmol, 1.0 equiv.) was added 4M HCl in dioxane (6.397 mL, 25.588 mmol, 50 equiv.) at room temperature. The reaction mixture was kept at room temperature for 1 hour and then concentrated. Compound 4 was used directly without further purification. LC-MS: [M+2H]/2 calculated 1220.28, found 1221.87.

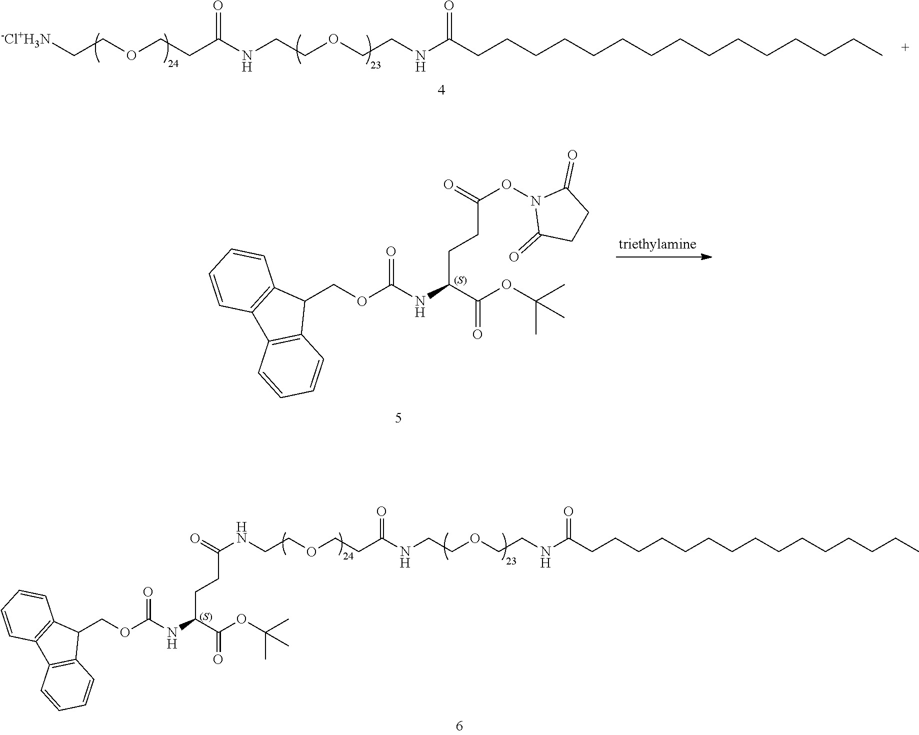

To a solution of compound 4 (1350 mg, 0. mmol, 1.0 equiv.) and compound 5 (327 mg, 0.626 mmol, 1.15 equiv.) in anhydrous DCM (10 mL) was added triethylamine (0.231 mL, 1.625 mmol, 3.0 equiv.) at room temperature. The reaction mixture was kept at room temperature overnight and then concentrated. Compound 6 was purified by CombiFlash® eluting with 12-20% methanol in dichloromethane. LC-MS: calculated [M+3H]/3 949.58, found 950.77.

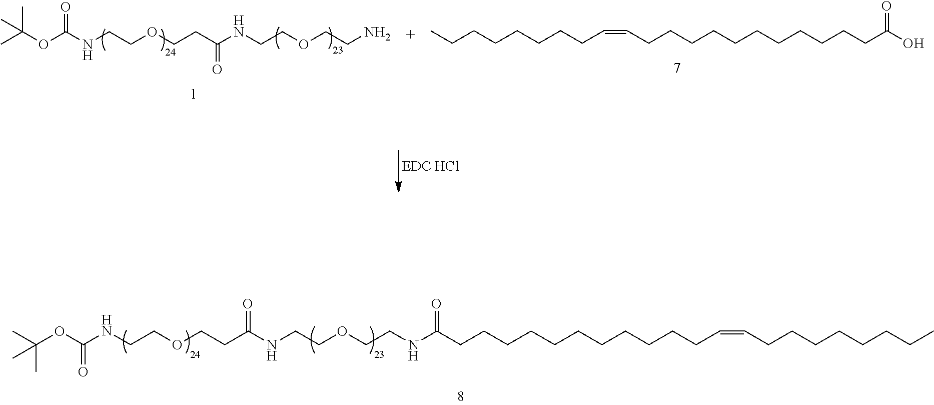

To a solution of compound 1 (1500 mg, 0.6517 mmol, 1.0 equiv.) and compound 7 (265 mg, 0.782 mmol, 1.2 equiv.) in anhydrous DCM (10 mL) was added EDC HCl (192 mg, 0.997 mmol, 1.5 equiv.) at room temperature. The reaction mixture was kept at room temperature for 3 hours and then concentrated. The product compound 8 was purified by CombiFlash® eluting with 12-20% methanol in dichloromethane. LC-MS: calculated [M+2H]+/2 1311.35, found 1311.87.

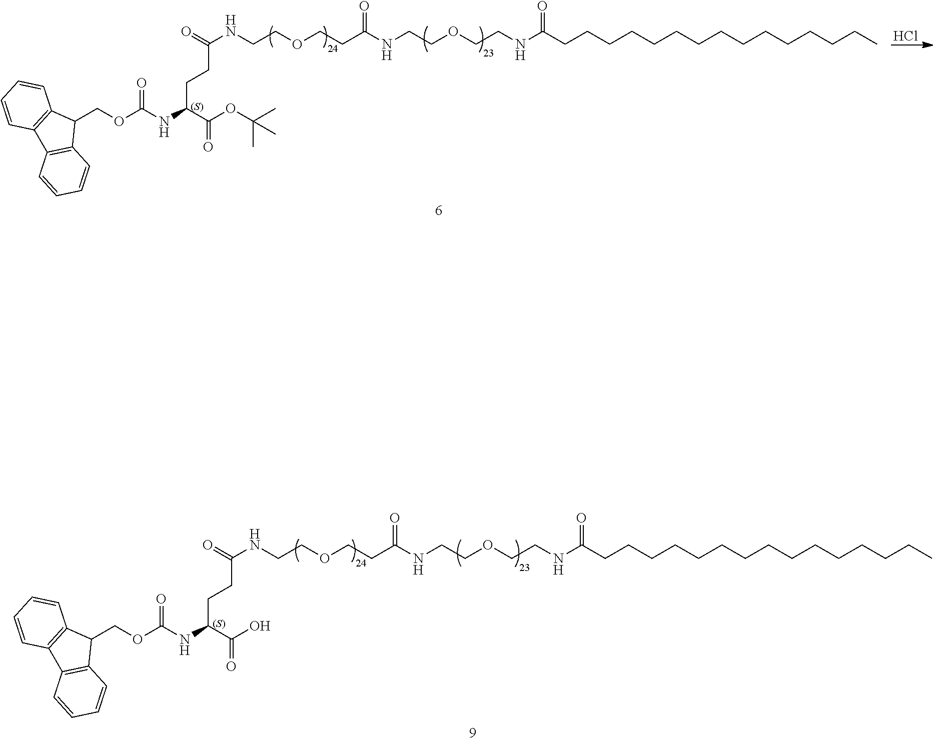

To compound 6 (1220 mg, 0.428 mmol, 1.0 equiv.) was added 4M HCl in dioxane (2.142 mL, 8.568 mmol, 20 equiv.) at room temperature. The reaction mixture was kept at room temperature for 5 hours. The reaction mixture was then concentrated. Compound 9 was used directly without further purification. LC-MS: [M+3H]/3 calculated 930.89, found 932.29.

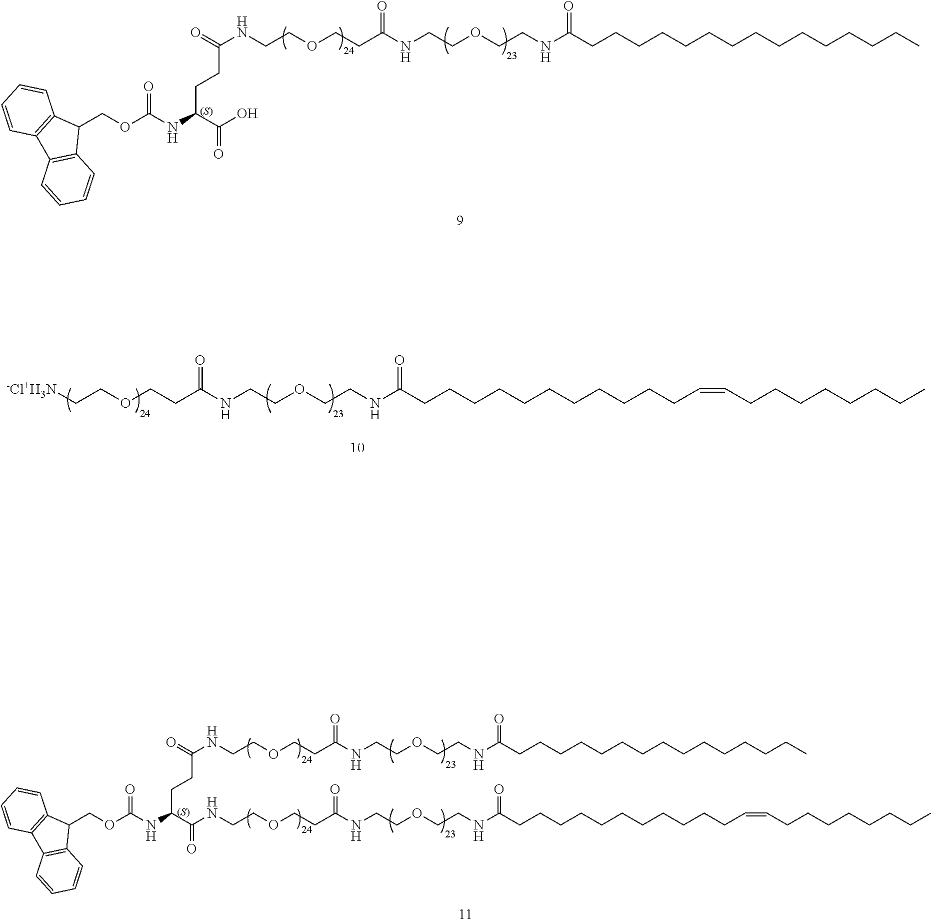

To a solution of compound 9 (800 mg, 0.286 mmol, 1.0 equiv.), compound 10 (prepared under conventional deprotection conditions from compound 8; 733 mg, 0.286 mmol, 1.00 equiv.), and diisopropylethylamine (0.150 mL, 0.859 mmol, 3.0 equiv.) in DMF (10 mL) was added TBTU (110 mg, 0.344 mmol, 1.2 equiv.) at room temperature. The reaction mixture was kept at room temperature for 3 hours. The reaction mixture was then concentrated. Compound 11 was purified by CombiFlash® eluting with 10-20% methanol in dichloromethane. LC-MS: [M+5H]/5 calculated 1059.46, found 1060.94.

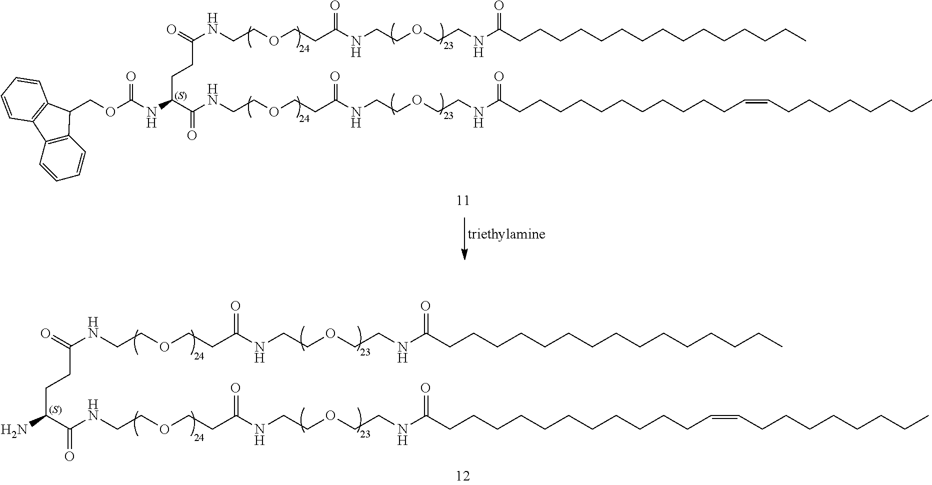

To a solution of compound 11 (914 mg, 0.172 mmol, 1.0 equiv.) in anhydrous DMF (4 mL) was added triethylamine (1 mL) at room temperature. The reaction mixture was kept at room temperature overnight and the solvent was removed under vacuum. compound 12 was used directly without further purification. LC-MS: [M+5H]/5 calculated 1015.05, found 1016.41.

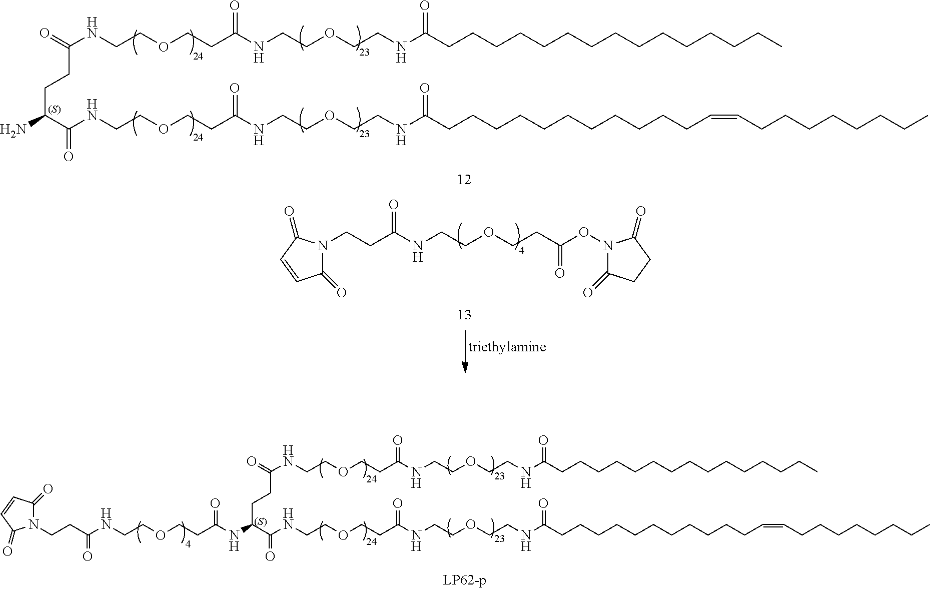

To a solution of compound 12 (875 mg, 0.172 mmol, 1.0 equiv.) and compound 13 (97.5 mg, 0.189 mmol, 1.1 equiv.) in anhydrous DCM (20 mL) was added triethylamine (0.073 mL, 0.517 mmol, 3.0 equiv.) at room temperature. The reaction mixture was kept at room temperature overnight and the solvent was concentrated. LP62-p was purified by CombiFlash® eluting with 12-20% methanol in dichloromethane. LC-MS: calculated [M+5H]/5 1094.68, found 1095.98.

### Synthesis of LP87-p

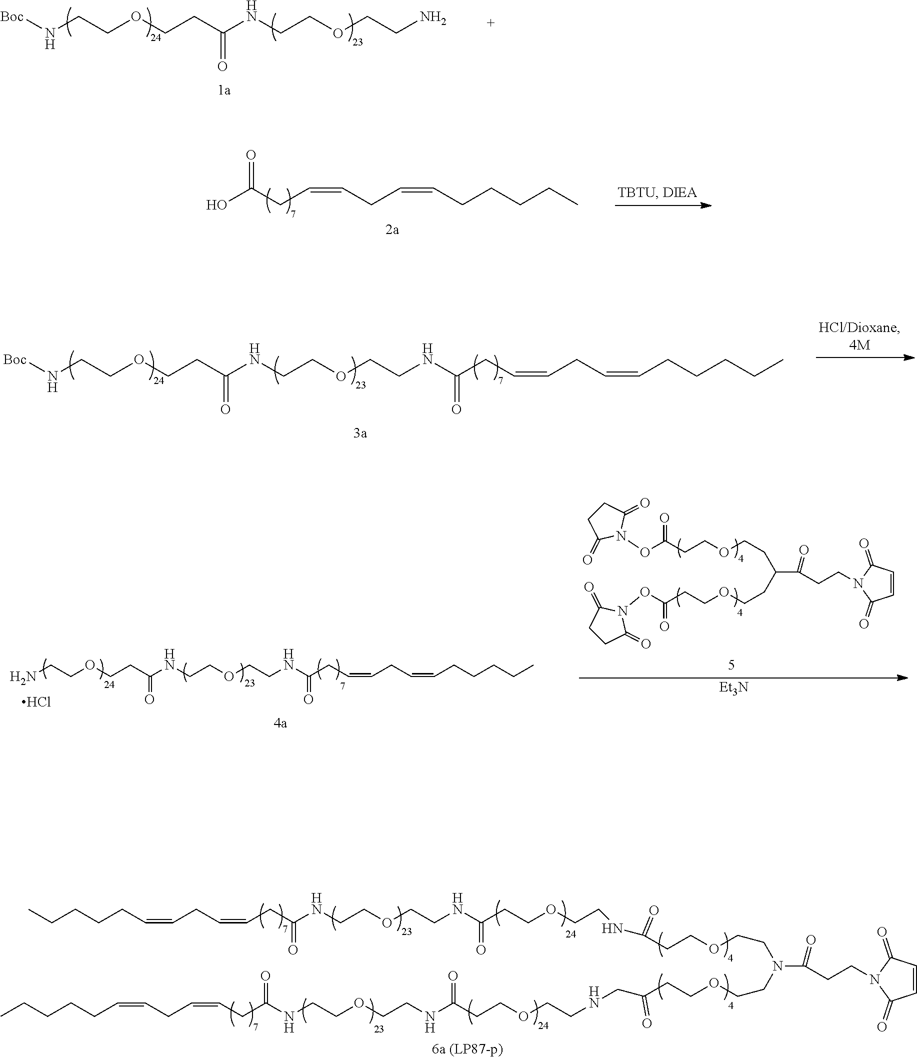

Solid TBTU (50 mg, 0.156 mmol) was added to a solution of Boc-protected PEG₄₇-amine 1a (Quanta Biodesign Limited, 300 mg, 0.13 mmol), linoleic acid 2a (37 mg, 0.13 mmol), and DIEA (68 μL mL, 0.39 mmol) in DMF (9 mL). The reaction mixture was sonicated to dissolve solids and stirred for 16 hours at room temperature. The solvent was removed under vacuum, and toluene was evaporated twice from the residue. The residue was dissolved in chloroform (50 mL), washed with NaHCO₃ (2×10 mL) and brine (10 mL). The product was dried (Na₂SO₄), concentrated under vacuum, and purified on CombiFlash® (SiO₂) using the system 0-20% MeOH in DCM, gradient 0-80%, 20 minutes. Calculated MW 2564.22, (M+2×18)/2=1300.1, (M+3×18)/3=872.74 Found: MS (ES, pos): 1299.74 [M+2NH₄]²⁺, 873.04 [M+3NH₄]³⁺. Compound 3a (195 mg, 0.0764 mmol) was converted into amine hydrochloride 4b by treatment with ice-cold 4M HCl/dioxane solution (5 mL) followed by stirring at room temperature for 1 hour. The reaction mixture was concentrated and dried under vacuum, the residual HCl was removed by 2 evaporations of toluene from the product. The dry amine hydrochloride salt was dissolved in anhydrous DMF (5 mL), Bis-NHS ester 5 (28 mg, 0.033 mmol) and Et₃N (28 uL, 0.198 mmol) were added and stirred for 3 hours at room temperature. The solvent was removed under vacuum, toluene was evaporated twice from the residue and the product 6a (LP87-p) was purified on CombiFlash® using the system 0-20% MeOH in DCM, gradient 0-100%, 30 min. Calculated MW 5556.9, (M+3×18)/3=1870.50, (M+4×18)/4=1407.23 Found: MS (ES, pos): 1870.50 [M+3NH₄]³⁺, 1407.40 [M+4NH₄]⁴⁺.

### Synthesis of LP89-p

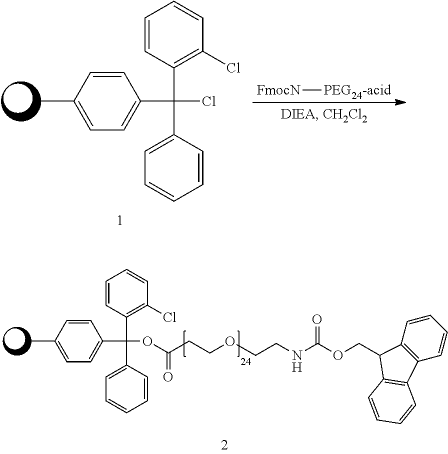

To a 25 mL fritted peptide synthesis vessel was added 2-chlorotrityl chloride resin 1 (0.4589 g, 1.46 mmol/g, 0.670 mmol). The resin was swelled in DCM and drained before adding Fmoc-N-amido-PEG₂₄-acid (0.9170 g, 0.670 mmol, 1 eq.) and diisopropylethylamine (DIEA) (0.584 mL, 3.35 mmol, 5 eq). The flask was rocked for 1 hour before adding methanol (0.367 mL, 0.8 mL/g resin) to cap any remaining trityl resin. After 40 minutes, the flask was drained, and washed with DCM three times, DMF two times, DCM two times, and MeOH three times (approximately 5 mL each wash). The resin was dried under high-vacuum overnight.

Resin loading: 11.5 mg of resin was suspended in 0.8 mL DMF and swelled for 15 minutes. 0.2 mL piperidine was added and the mixture was allowed to stand 15 minutes. A 10× dilution was taken up in DMF and a UV-vis spectra was taken, A=2.66 (approximately). The resin loading was calculated to be 0.297 mmol/g, with a total of 919 mg of resin, for a scale of 0.273 mmol.

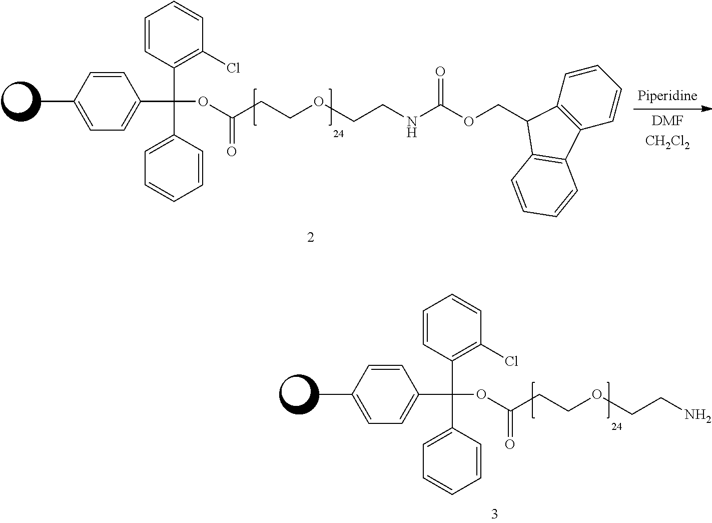

The resin 2 was suspended in DCM/DMF/piperidine 1:1:2, 9.6 mL. After shaking for 30 minutes, the solution was drained, and resin washed with DMF (4×9.2 mL).

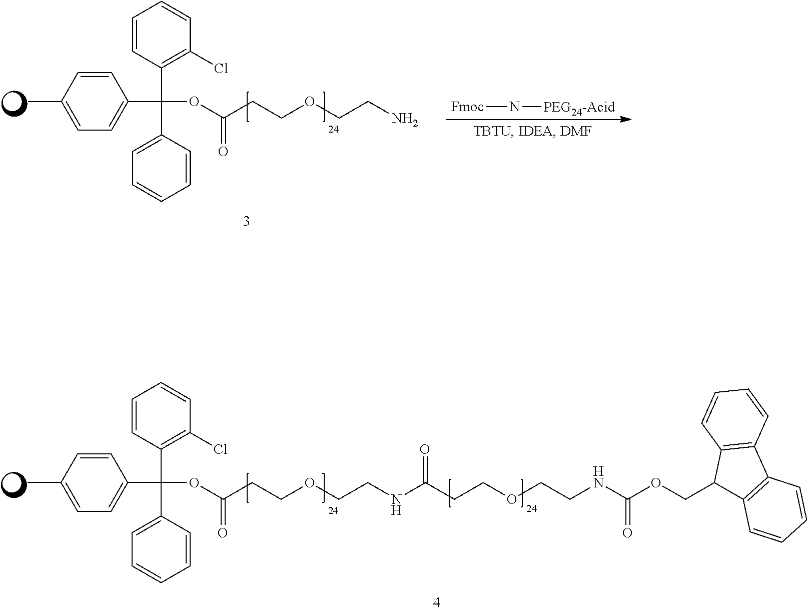

Fmoc-N-amido-PEG24-acid (0.7473 g, 0.5460 mmol, 2 eq), TBTU (0.1753 g, 0.5460 mmol, 2 eq), and DIEA (0.190 mL, 1.092 mmol, 4 eq) were combined in DMF (7.6 mL) and mixed for 2-3 minutes before the solution was added to the resin in the synthesis flask. The flask was shaken for 1 hour, after which a yellow orange solution was drained from the orange resin. The resin was washed with DMF and MeOH (3×8.6 mL each) then dried overnight under high-vacuum. 1.277 g resin, theoretical 1.227 g. Product masses were observed by LC-MS following a microcleavage.

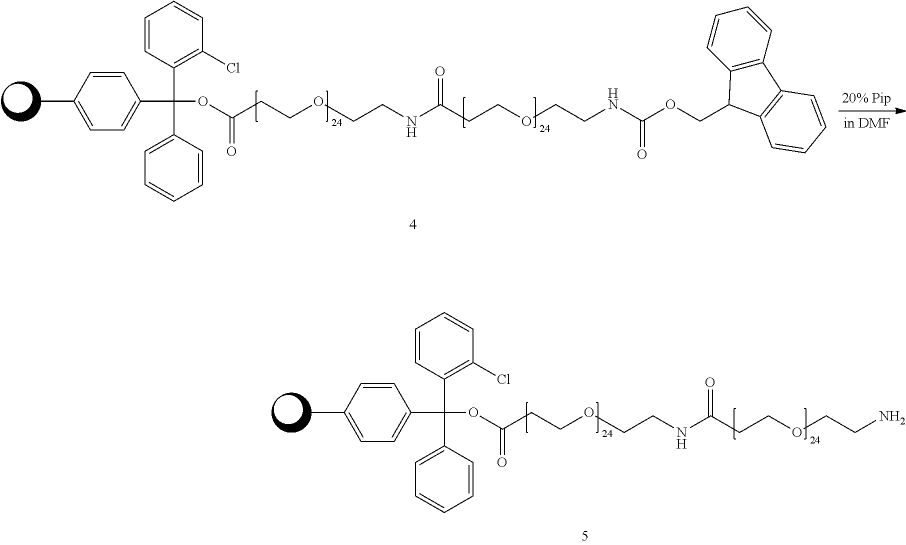

The resin was treated with 20% piperidine in DMF (12.3 mL) for 30 minutes, then washed with DMF (4×12.3 mL).

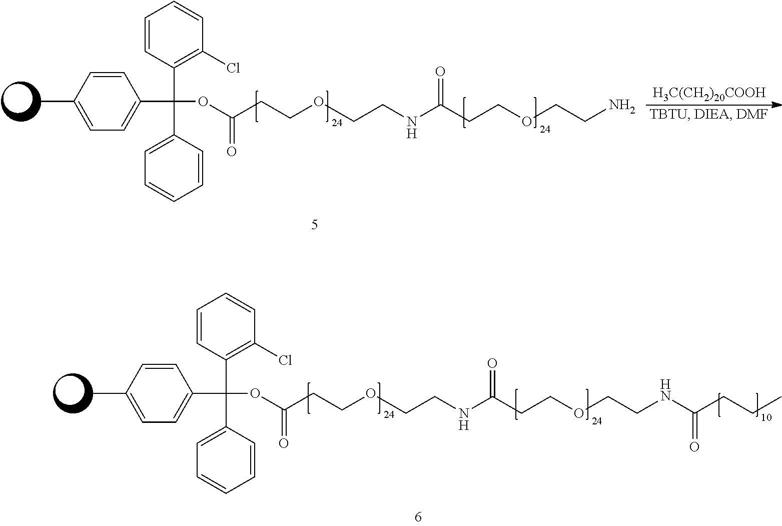

Behenic acid (0.186 g, 0.546 mmol, 2.0 eq), TBTU (0.175 g, 0.546 mmol, 2 eq) and DIEA (0.190 mL, 1.092 mmol, 4 eq) were dissolved in DMF (10.7 mL). The solution was added to the resin. The solution vial was rinsed with DMF and added to the resin (2×1 mL). The mixture was shaken for 75 minutes then drained and washed with DMF, THF, and MeOH (3×13 mL each). The resin was dried under high-vacuum (90 minutes). 1.351 g obtained, theoretical 1.254 g. Product masses (and no starting material masses) were observed in LC-MS following a microcleavage.

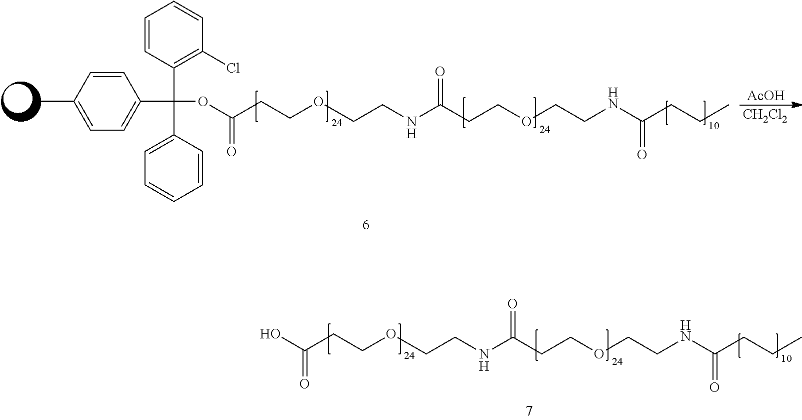

The resin was treated with DCM (11 mL) and AcOH (1.1 mL) for 30 minutes, then drained. This cleavage was repeated a total of 4 times, then the resin was treated with 8 mL CH₂Cl₂, 1 mL AcOH, and 1 mL 2,2,2-trifluoroethanol, shaken for 30 minutes, and drained. This cleavage was repeated a second time. The solutions from all cleavages were combined and concentrated to yield 530.8 mg, which was purified by column chromatography.

The crude compound was loaded onto a silica column (24 g) and eluted 0-20% MeOH in CH₂Cl₂. Clean fractions were combined to yield 69.9 mg of target compound.

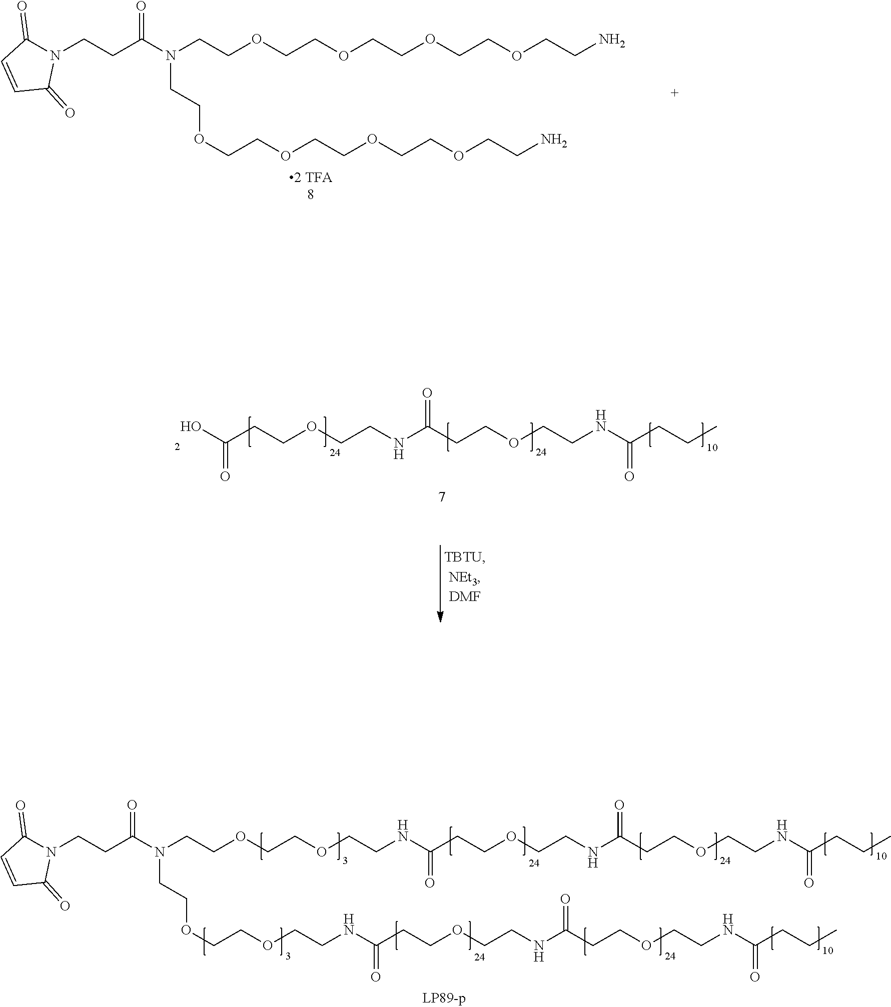

To a vial was added N-mal-N-bis(PEG4)amine TFA salt (10.7 mg, 0.0128 mmol, 1 eq), acid-PEG₂₄-amido-PEG₂₄-C₂₂ (69.9 mg, 0.0269 mmol, 2.1 eq), TBTU (10.3 mg, 0.0320 mmol, 2.5 eq), NEt₃ (5.4 uL, 0.0385 mmol, 3 eq), and CH₂Cl₂ (1 mL). The reaction mixture was stirred for 24 hours, then NEt₃ (5.4 uL, 0.0385 mmol, 3 eq) was added. After approximately 50 hours, the reaction mixture was concentrated and purified by column chromatography, 0-30% MeOH in DCM, to obtain 32.8 mg of LP89-p (44%).

### Synthesis of LP90-p

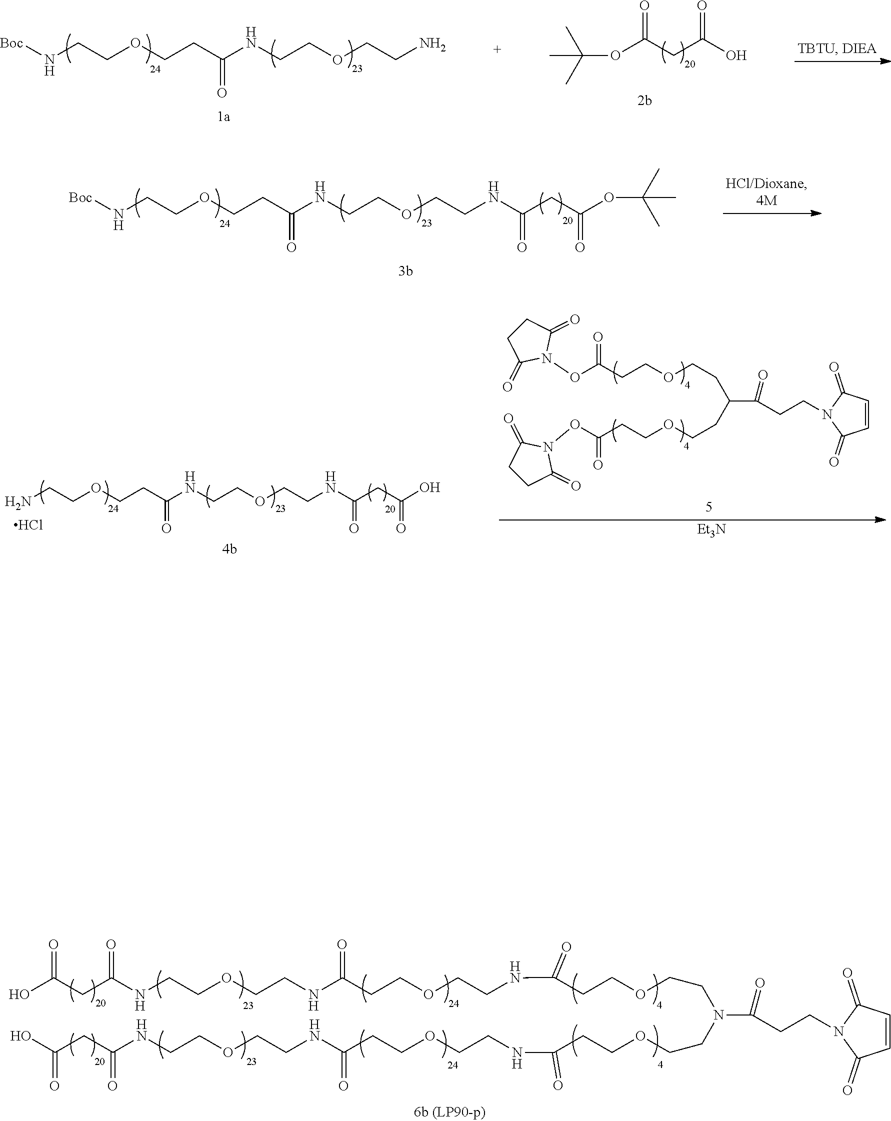

Solid TBTU (50 mg, 0.156 mmol) was added to a solution of Boc-protected PEG-amine 1a (Quanta Biodesign Limited, 300 mg, 0.13 mmol), mono-protected docosanedioic acid 2b (56 mg, 0.13 mmol), and DIEA (68 uL mL, 0.39 mmol) in DMF (9 mL). The reaction mixture was stirred for 16 hours at room temperature. The solvent was removed under vacuum and toluene was evaporated 3 times from the residue. The residue was taken in DCM 30 (mL), mixed with SiO₂ (1.6 g), and loaded on CombiFlash®. The product was purified using the system 0-20% MeOH in DCM, gradient 0-100%, 45 minutes. Calculated MW 2710.45, (M+2×18)/2=1373.22, (M+3×18)/3=921.48 Found: MS (ES, pos): 1373.18 [M+2NH₄]²⁺, 921.37 [M+3NH₄]³⁺.

Compound 3b (238 mg, 0.088 mmol) was converted into amino acid hydrochloride 4b by treatment with ice-cold 4M HCl/dioxane solution (6 mL) followed by stirring at room temperature for 4 hours. The reaction mixture was concentrated and dried under vacuum, the residual HCl was removed by 2 evaporations of toluene from the residue.

The dry amine hydrochloride salt 4b was dissolved in anhydrous DCM (5 mL), Bis-NHS ester 5 (34.2 mg, 0.04 mmol) and Et₃N (55 uL, 0.4 mmol) were added and stirred for 3 hours at room temperature. The solvent was removed under vacuum, and the product 6b (LP90-p) was purified on CombiFlash® using the system 0-20% MeOH in DCM, gradient 0-100%, 40 min. Calculated MW 5737.13, (M+3×18)/3=1930.38, (M+4×18)/4=1452.28 Found: MS (ES, pos): 1930.45 [M+3NH₄]³⁺, 1452.29 [M+4NH₄]⁴⁺.

### Synthesis of LP91-p

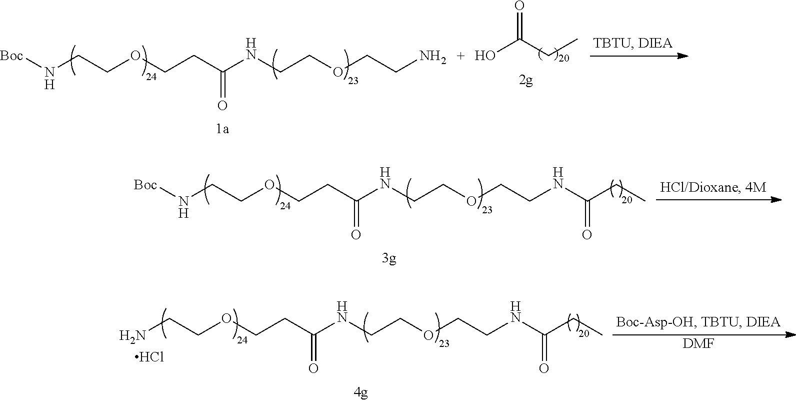

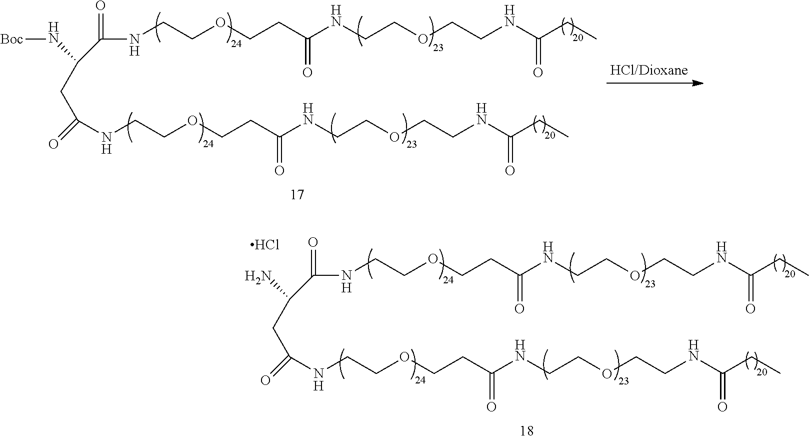

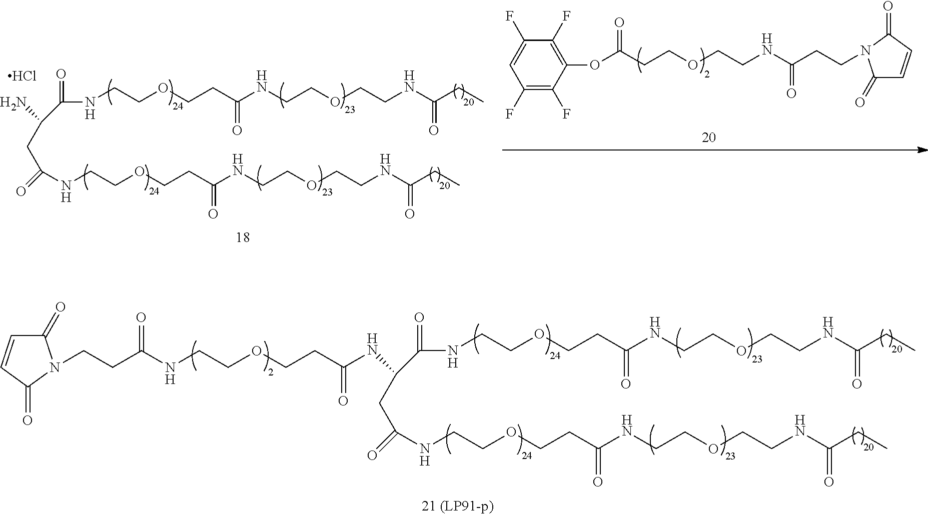

Solid TBTU (335 mg, 1.043 mmol) was added to a solution of Boc-protected PEG47-amine 1a (2 g, 0.869 mmol), behenic acid 2 g (296 mg, 0.87 mmol), and DIEA (454 uL mL, 2.067 mmol) in DMF (16 mL). The reaction mixture was sonicated to dissolve solids and stirred for 16 hours at room temperature. The solvent was removed under vacuum and toluene was evaporated twice from the residue. The residue was dissolved in chloroform (150 mL) and washed with NaHCO₃ (2×30 mL) and brine (30 mL). Product 3 g was dried (Na₂SO₄), concentrated under vacuum, and purified on CombiFlash® (SiO₂) using the system 0-20% MeOH in DCM, gradient 0-80%, 35 minutes. Calculated MW 2624.36, (M+2×18)/2=1330.18, (M+3×18)/4=892.79. Found: MS (ES, pos): 1330.58 [M+2NH₄]²⁺, 893.21 [M+3NH₄]³⁺.

Compound 3g (1.862 g) was converted into amine hydrochloride 4 g by treatment with 4 M HCl in dioxane solution (10 mL) as described in the procedure for LP39-p, above.

An aliquot of dry salt 4 g (227 mg, 0.089 mmol)) was combined with Boc-Asp-OH (10 mg, 0.043 mmol), TBTU (32 mg, 0.099 mmol), and DIEA (96 uL, 0.55 mmol) as described in the preparation of LP54-p to obtain compound 17, yield 152 mg (0.029 mmol). This product was treated with HCl/dioxane solution as in preparation of 14c as described in the synthesis of LP54-p, above, to obtain hydrochloride salt 18 (yield 100%), used directly in the following step. Calculated MW 5145.57, (M+3)/3=1716.19, (M+4)/4=1287.23. Found: MS (ES, pos): 1715.91 [M+3H]³⁺, 1287.23 [M+4H]⁴⁺.

Hydrochloride salt 18 (0.029 mmol) was combined with tetrafluorophenyl ester 20 (Quanta Biodesign, 15 mg, 0.032 mmol) and Et₃N (12 uL, 0.087 mmol) as described for 16c in the synthesis of LP54-p, above. The product 21 (LP91-p) was purified on CombiFlash®. Yield 40 mg. Calculated MW 5455.87, (M+4)/4=1364.97, (M+5)/5=1092.17. Found: MS (ES, pos): 1364.66 [M+4H]⁴⁺, 1092.05 [M+4H]⁴⁺.

### Synthesis of LP92-p

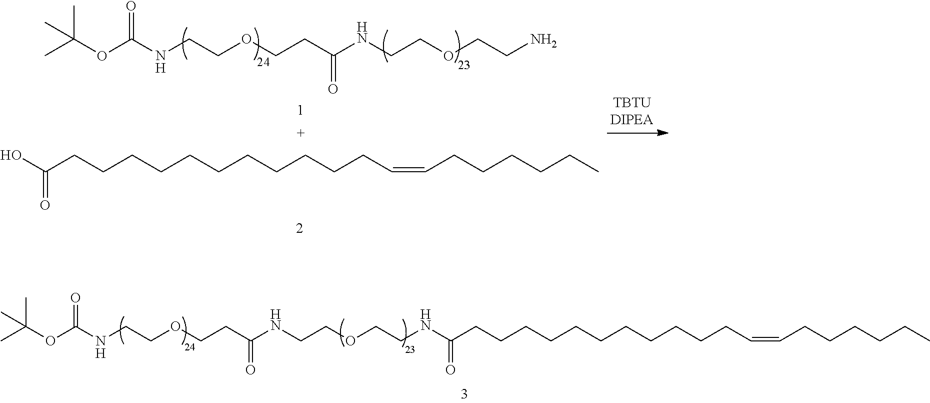

To a solution of compound 1 (140 mg, 0.0608 mmol, 1.0 equiv.), compound 2 (20.8 mg, 0.0669 mmol, 1.1 equiv.), and diisopropylethylamine (0.032 mL, 0.182 mmol, 3.0 equiv.) in anhydrous DMF (3 mL) was added TBTU (23.4 mg, 0.073 mmol, 1.2 equiv.) at room temperature. The reaction mixture was kept at room temperature for 2 hours. The reaction mixture was concentrated. Compound 3 was purified by CombiFlash® eluting with 12-18% methanol in dichloromethane. LC-MS: calculated [M+2H]+/2 1297.33, found 1297.19.

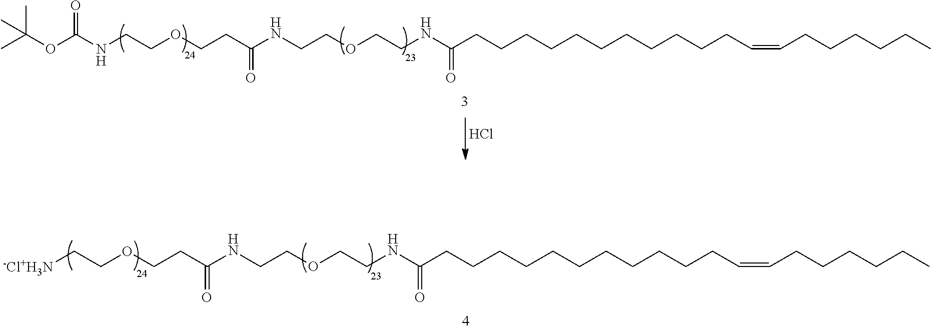

To solid of compound 3 (90 mg, 0.0347 mmol, 1.0 equiv.) was added HCl solution in dioxane (0.434 mL, 1.734 mmol, 50 equiv.) at room temperature. The reaction mixture was kept at room temperature for 30 minutes and was then concentrated. Compound 4 was used directly without further purification. LC-MS: calculated [M+2H]+/2 1247.30, found 1247.98.

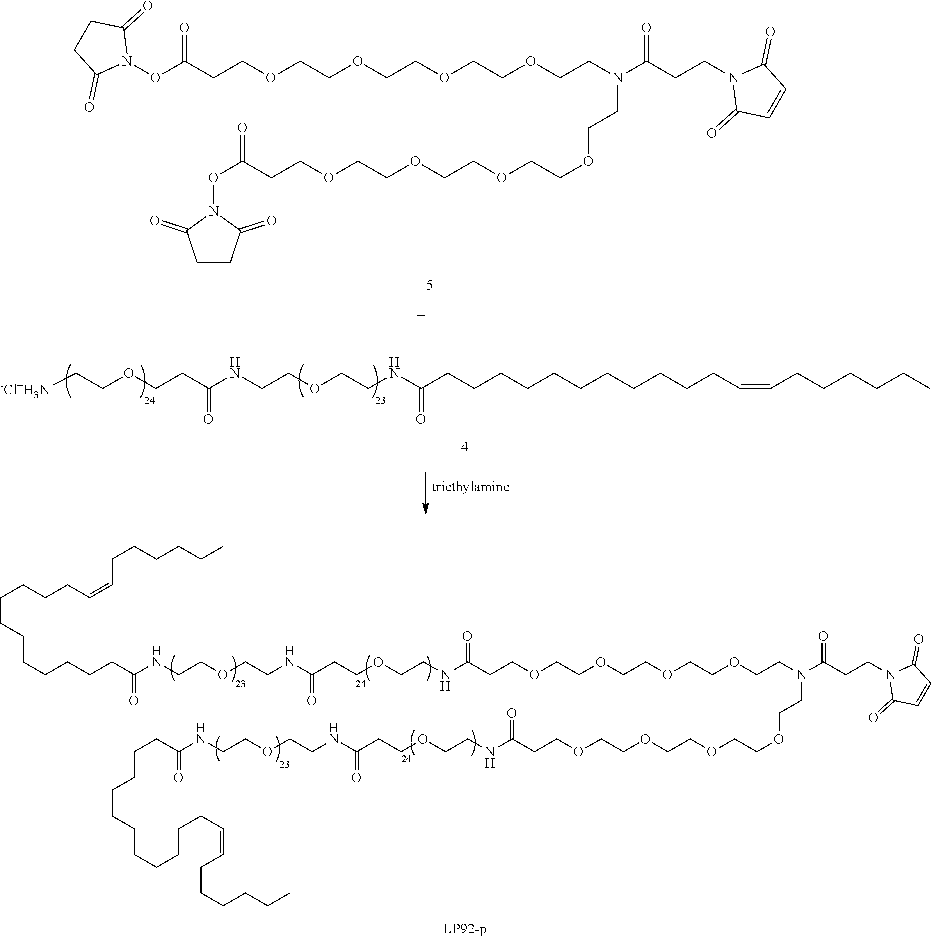

To a solution of compound 5 (14 mg, 0.0163 mmol, 1.0 equiv.) and compound 4 (84.6 mg, 0.0334 mmol, 2.05 equiv.) in anhydrous DCM (2 mL) was added triethylamine (0.012 mL, 0.0815 mmol, 5.0 equiv.) at room temperature. The reaction mixture was kept at room temperature for 1 hour and the solvent was removed under vacuum. LP92-p was purified by CombiFlash® eluting with 12-18% methanol in dichloromethane. LC-MS: calculated [M+5H]+/5 1123.70, found 1124.10, calculated [M+6H]+/6 936.58, found 937.22.

### Synthesis of LP93-p

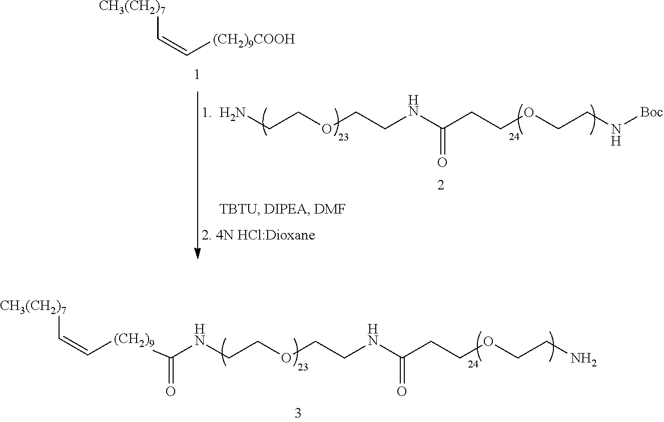

To cis-11-eicosenoic acid 1 (30 mg, 0.0979 mmol) in a solution of Boc-PEG47-NH2 2 (223 mg, 0.1 mmol) in DMF (2.0 mL) was added TBTU (37.2 mg, 0.115 mmol) and DIPEA (50 uL). After stirring the resulting suspension overnight, water was added. The mixture was extracted using DCM:20% TFE and the combined organic phases were dried over Na₂SO₄. After filtration, the solvent was removed under vacuum to dryness and the crude product was purified by flash chromatography (20% MeOH in DCM). To the product was added 2 mL of 4N HCl:Dioxane under anhydrous conditions until the deprotection was determined to be complete by LC-MS: calculated [M+H]+ for 2550.28 m/z, found 2551.

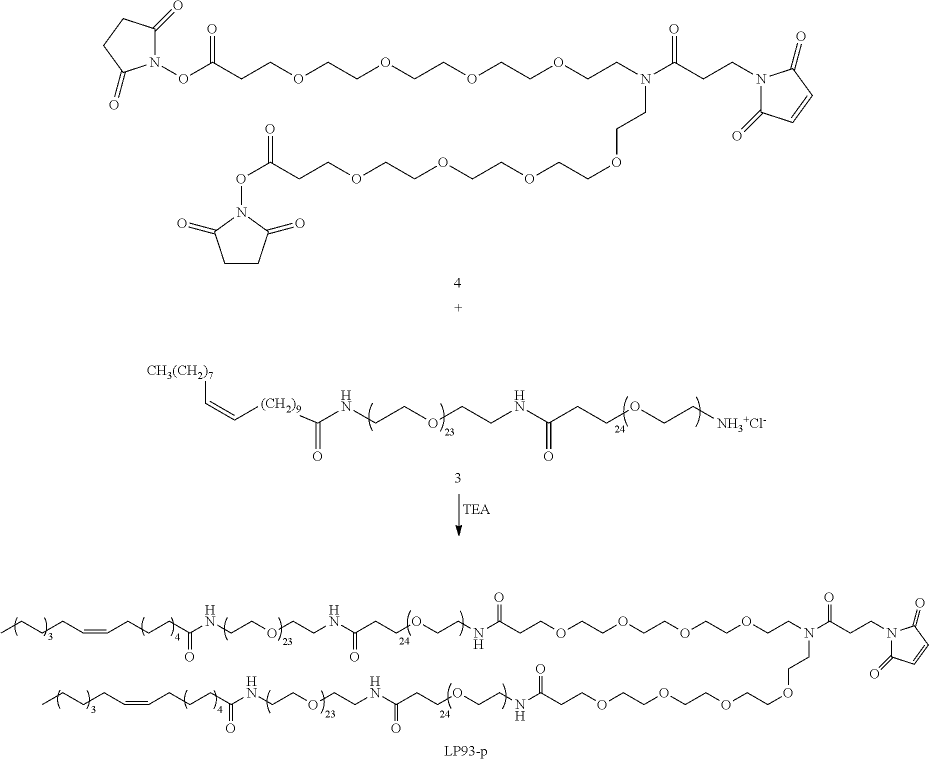

To a solution of compound 4 (19 mg, 0.0221 mmol, 1.0 equiv.) and compound 3 (16 mg, 0.0454 mmol, 2.05 equiv.) in anhydrous DCM (2 mL) was added triethylamine (16 uL, 0.1106 mmol, 5.0 equiv.) at room temperature. The reaction mixture was kept at room temperature overnight and the solvent was removed under vacuum. LP93-p was purified by CombiFlash® eluting with 10-17% methanol in dichloromethane.

### Synthesis of LP94-p

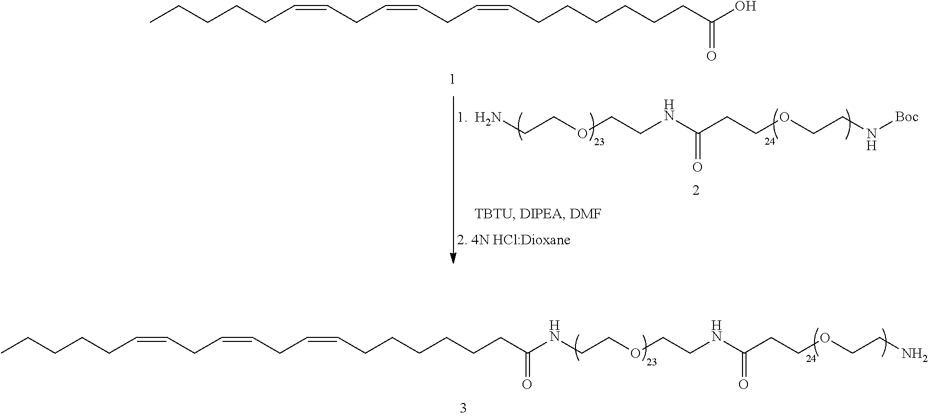

To dihomo-γ-linolenic acid 1 (30 mg, 0.0979 mmol) in a solution of DMF (2.0 mL) was added Boc-PEG47-NH₂ 2 (225 mg, 0.1 mmol), TBTU (37.7 mg, 0.117 mmol) and DIPEA (50 uL). After stirring the resulting suspension overnight, water was added. The mixture was extracted using DCM:20% TFE and the combined organic phases were dried over Na₂SO₄. After filtration, the solvent was concentrated to dryness and the crude product was purified by flash chromatography (DCM:20% MeOH). To the product was added 2 mL of 4N HCl:Dioxane under anhydrous conditions until the deprotection was determined to be complete by LC-MS: calculated [M+H]+ for 2560.28 m/z, found 2561.01.

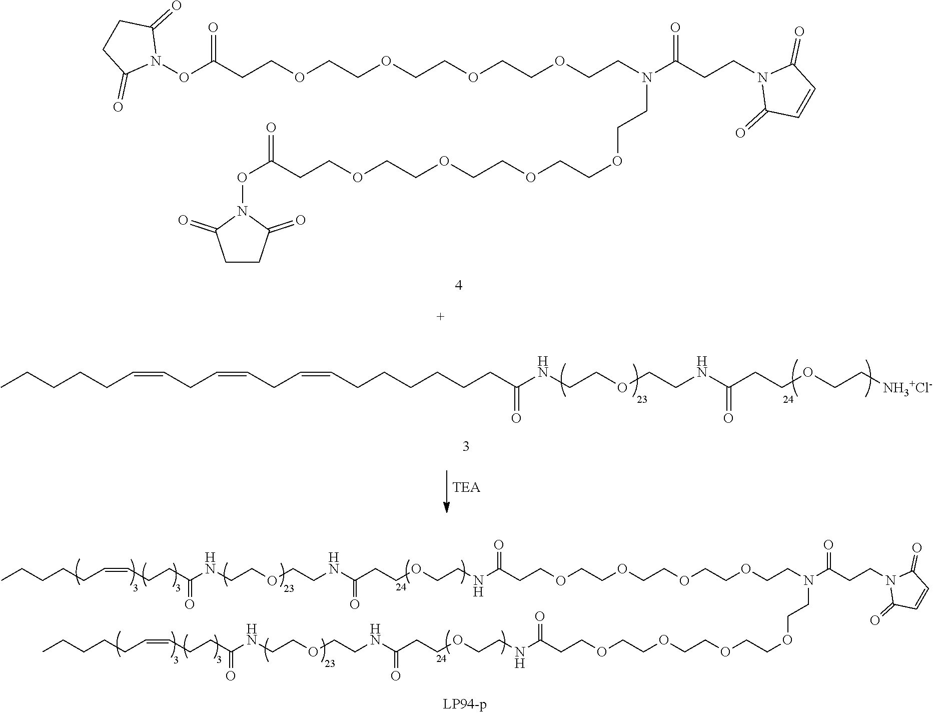

To a solution of compound 4 (19 mg, 0.0221 mmol, 1.0 equiv.) and compound 3 (112 mg, 0.0454 mmol, 2.05 equiv.) in anhydrous DCM (2 mL) was added triethylamine (16 uL, 0.1106 mmol, 5.0 equiv.) at room temperature. The reaction mixture was kept at room temperature overnight and the solvent was removed under vacuum. LP94-p was separated by CombiFlash® eluting with 10-17% methanol in dichloromethane.

### Synthesis of LP95-p

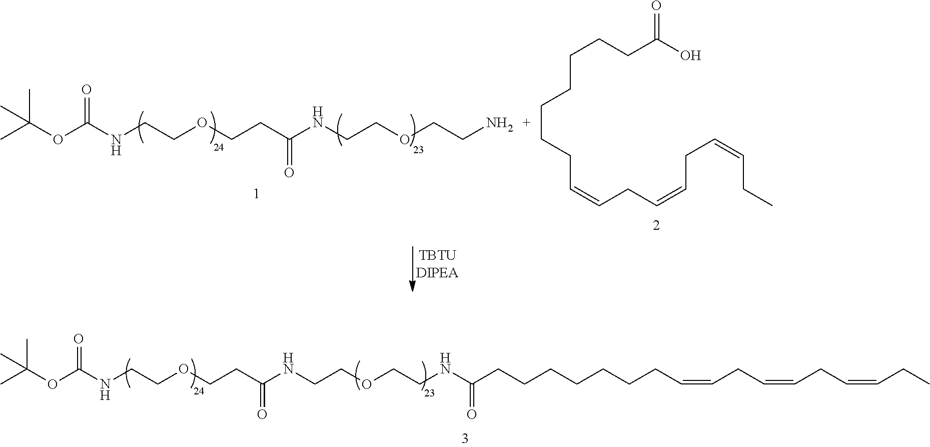

To a solution of compound 1 (150 mg, 0.0652 mmol, 1.0 equiv.), compound 2 (20 mg, 0.0717 mmol, 1.1 equiv.) and diisopropylethylamine (0.034 mL, 0.195 mmol, 3.0 equiv.) in anhydrous DMF (3 mL) was added TBTU (25.1 mg, 0.0782 mmol, 1.2 equiv.) at room temperature. The reaction mixture was kept at room temperature for 2 hours. The reaction mixture was then concentrated. Compound 3 was purified by CombiFlash® eluting with 12-18% methanol in dichloromethane. LC-MS: calculated [M+2H]+/2 1281.30, found 1281.71.

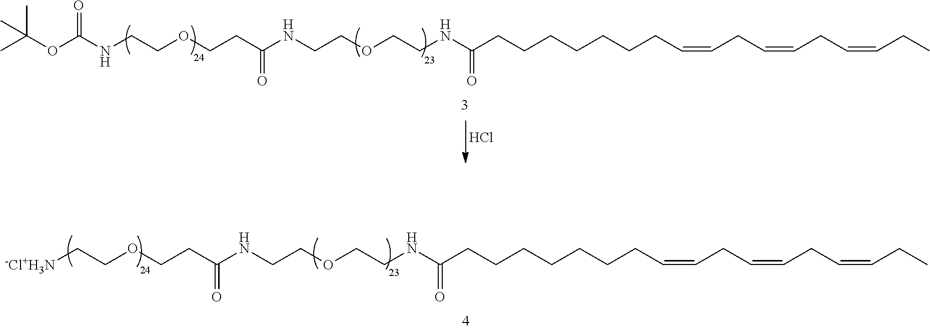

To compound 3 (80 mg, 0.0312 mmol, 1.0 equiv.) was added HCl solution in dioxane (0.390 mL, 1.561 mmol, 50 equiv.) at room temperature. The reaction mixture was kept at room temperature for 30 minutes and the solvent was removed under vacuum. Compound 4 was used directly without further purification. LC-MS: calculated [M+2H]+/2 1231.27, found 1231.65.

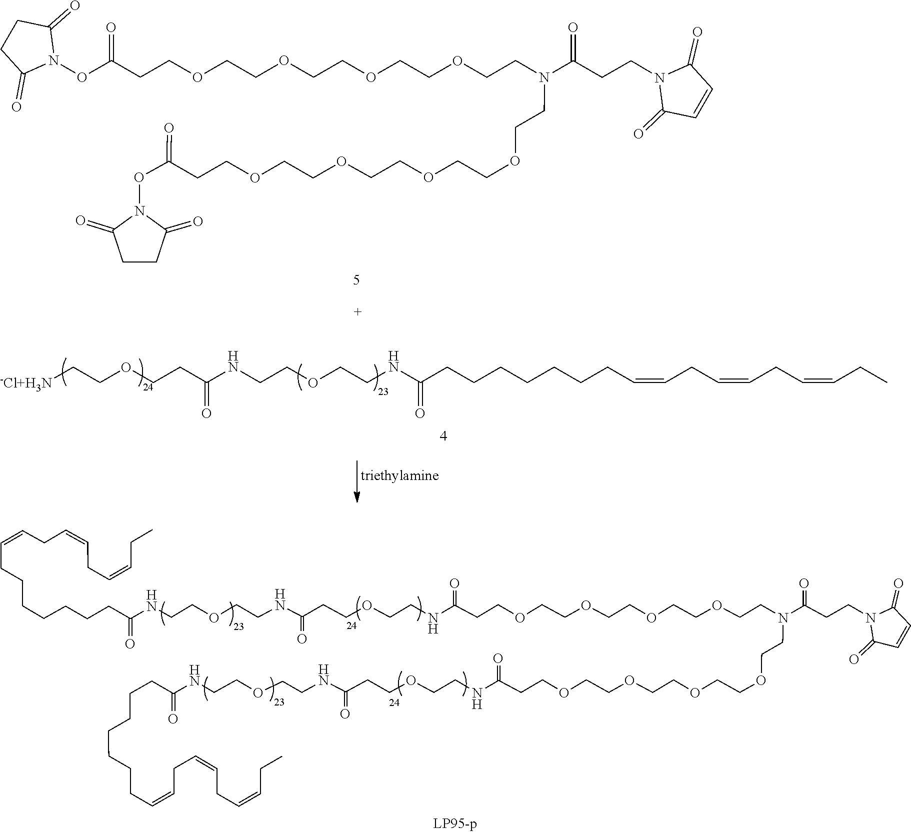

To a solution of compound 5 (13 mg, 0.0151 mmol, 1.0 equiv.) and compound 4 (77.5 mg, 0.0310 mmol, 2.05 equiv.) in anhydrous DCM (2 mL) was added triethylamine (0.011 mL, 0.0757 mmol, 5.0 equiv.) at room temperature. The reaction mixture was kept at room temperature for 1 hour and the solvent was removed under vacuum. LP95-p was purified by CombiFlash® eluting with 12-18% methanol in dichloromethane. LC-MS: calculated [M+5H]+/5 1110.88, found 1111.62, calculated [M+6H]+/6 925.90, found 926.41.

### Synthesis of LP101-p

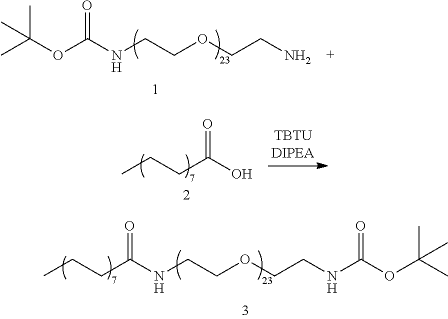

To a solution of compound 1 (250 mg, 0.213 mmol, 1.0 equiv.), compound 2 (65 mg, 0.255 mmol, 1.20 equiv.) and diisopropylethylamine (0.111 mL, 0.629 mmol, 3.0 equiv.) in anhydrous DMF (3 mL) was added TBTU (102 mg, 0.319 mmol, 1.2 equiv.) at room temperature. The reaction mixture was kept at room temperature overnight. Compound 3 was purified by CombiFlash® eluting with 6-12% methanol in dichloromethane. LC-MS: calculated [M+H]+ 1411.95, found 1411.95.

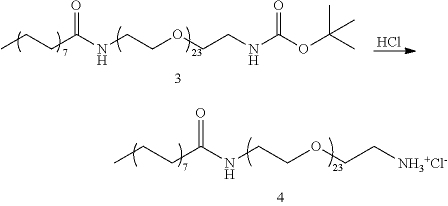

To solid of compound 3 (200 mg, 0.141 mmol, 1.0 equiv.) was added HCl solution in dioxane (0.708 mL, 2.833 mmol, 20 equiv.) at room temperature. The reaction mixture was kept at room temperature for 1 hour and the solvent was removed under vacuum. The product was used directly without further purification. LC-MS: calculated [M+H]+ 1311.90, found 1312.32.

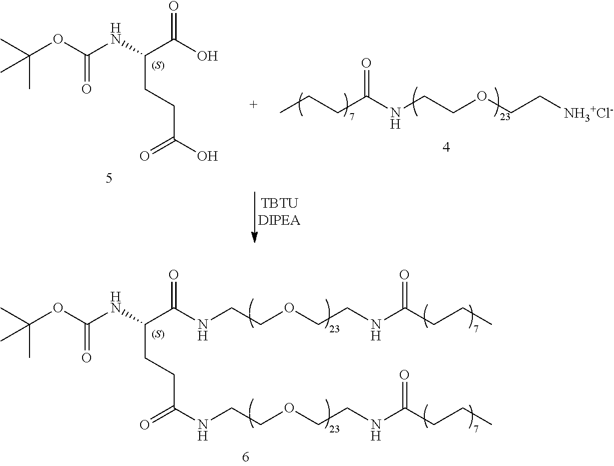

To a solution of compound 5 (100 mg, 0.0404 mmol, 1.0 equiv.), compound 4 (111 mg, 0.0829 mmol, 2.05 equiv.), and diisopropylethylamine (35 mL, 0.202 mmol, 3.0 equiv.) in anhydrous DMF (3 mL) was added TBTU (32.5 mg, 0.101 mmol, 2.5 equiv.) at room temperature. The reaction mixture was kept at room temperature overnight and was then concentrated. Compound 6 was purified by CombiFlash® eluting with 6-10% methanol in dichloromethane. LC-MS: calculated [M+2H]+/2 1417.44, found 1418.19.

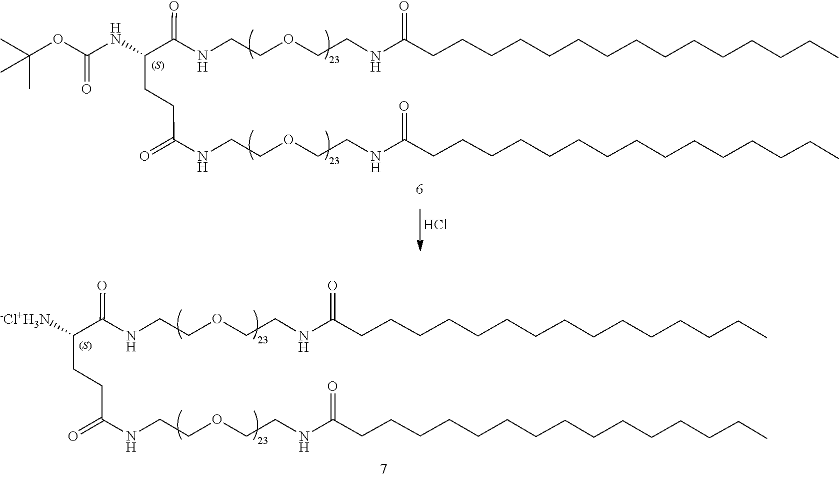

To compound 6 (80 mg, 0.0282 mmol, 1.0 equiv.) was added 4M HCl in dioxane (0.353 mL, 1.411 mmol, 50 equiv.) at room temperature. The reaction mixture was kept at room temperature for 1 hour and was then concentrated. Compound 7 was used directly without further purification. LC-MS: [M+2H]/2 calculated 1367.41, found 1368.26.

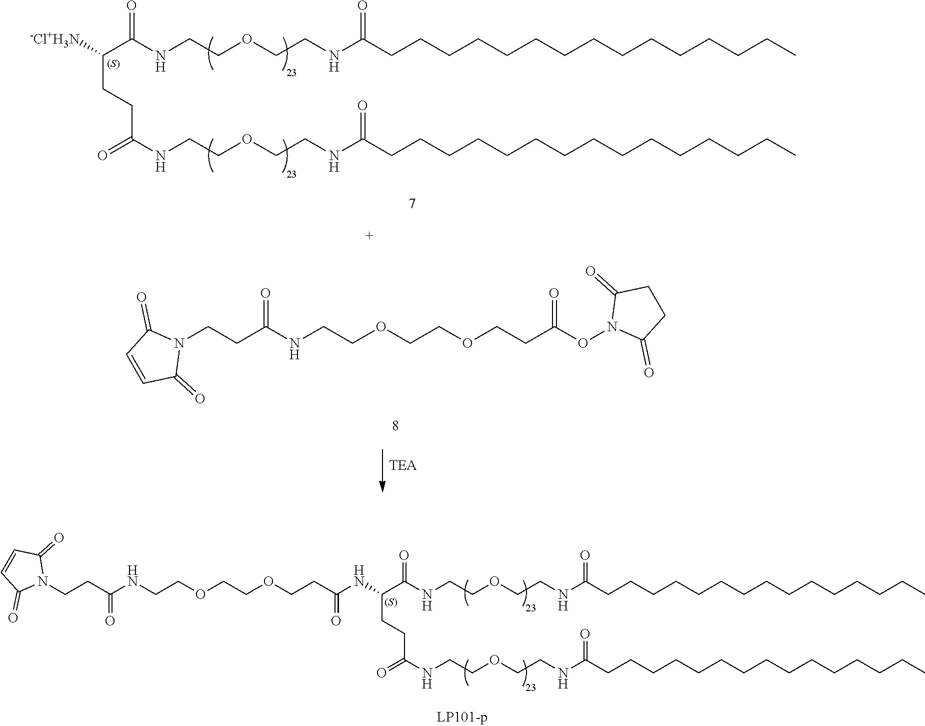

To a solution of compound 7 (78 mg, 0.0281 mmol, 1.0 equiv.) and compound 8 (12 mg, 0.0281 mmol, 1.0 equiv.) in anhydrous DCM (2 mL) was added triethylamine (0.020 mL, 0.140 mmol, 5.0 equiv.) at room temperature. The reaction mixture was kept at room temperature overnight and the solvent was concentrated. LP101-p was separated by CombiFlash® eluting with 12-20% methanol in dichloromethane. LC-MS: calculated [M+3H]/3 1015.31, found 1015.71.

### Synthesis of LP102-p

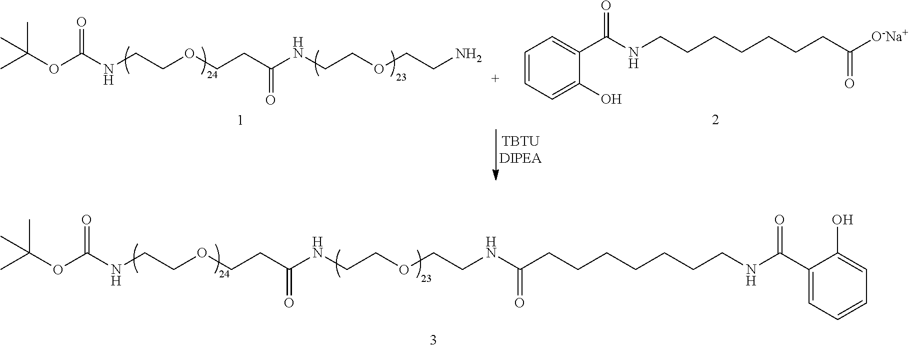

To a solution of compound 1 (124 mg, 0.0539 mmol, 1.0 equiv.), compound 2 (19.5 mg, 0.0646 mmol, 1.2 equiv.) and diisopropylethylamine (0.028 mL, 0.161 mmol, 3.0 equiv.) in anhydrous DMF (2 mL) was added TBTU (20.8 mg, 0.0646 mmol, 1.2 equiv.) at room temperature. The reaction mixture was kept at room temperature for 1 hour. The reaction mixture was quenched with saturated sodium bicarbonate aqueous solution. The aqueous phase was extracted with DCM (3×10 mL), and the combined organic phases were dried over Na₂SO₄, and concentrated. Compound 3 was purified by CombiFlash® eluting with 10-12% methanol in dichloromethane. LC-MS: calculated [M+2H]+/2 1281.76, found 1282.19.

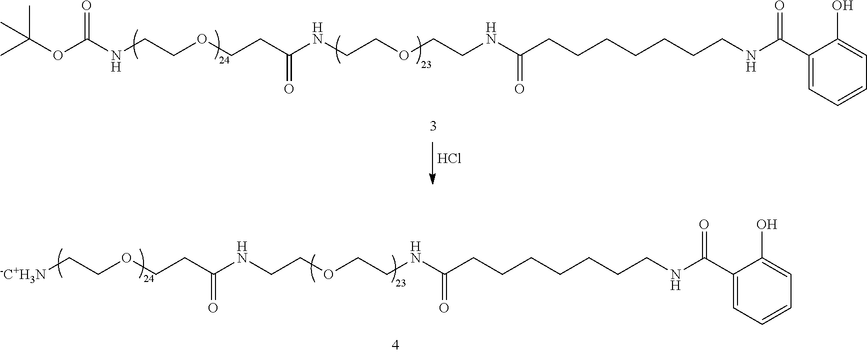

To compound 3 (66 mg, 0.0257 mmol, 1.0 equiv.) was added 4M HCl in dioxane (0.322 mL, 1.287 mmol, 50 equiv.) at room temperature. The reaction mixture was kept at room temperature for 1 hour and was then concentrated. Compound 4 was used directly without further purification. LC-MS: [M+2H]/2 calculated 1231.75, found 1232.01.

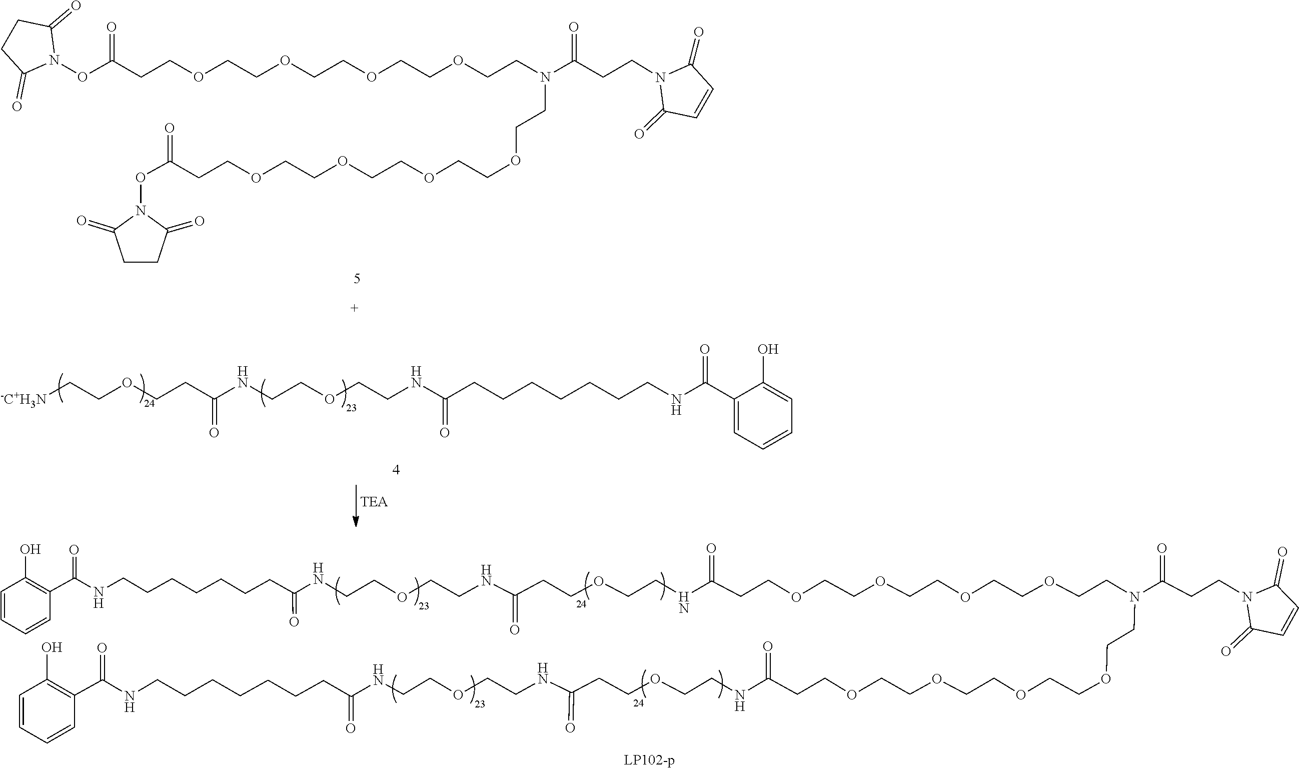

To a solution of compound 5 (11 mg, 0.0128 mmol, 1.0 equiv.) and compound 4 (64 mg, 0.0256 mmol, 2.00 equiv.) in anhydrous DCM (2 mL) was added triethylamine (0.009 mL, 0.064 mmol, 5.0 equiv.) at room temperature. The reaction mixture was kept at room temperature for 1 hour and the solvent was concentrated. LP102-p was separated by CombiFlash® eluting with 12-18% methanol in dichloromethane. LC-MS: calculated [M+6H]+/6 926.20, found 926.41.

### Synthesis of LP103-p

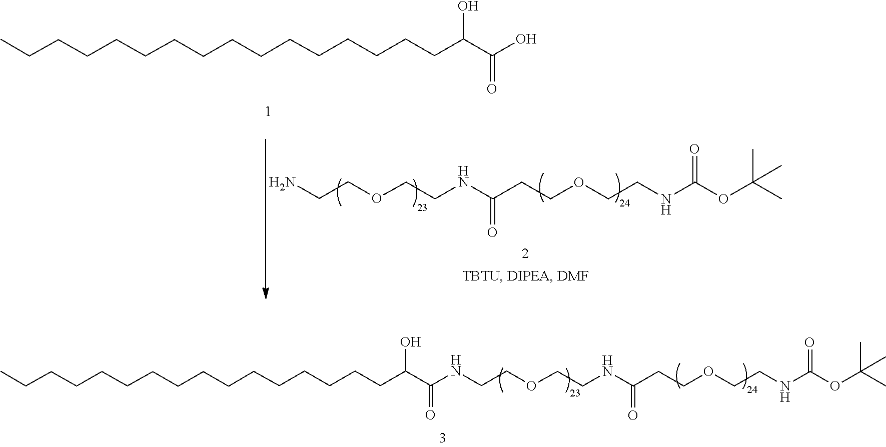

To compound 1 (35 mg, 0.1170 mmol) in a solution DMF (2.0 mL) was added Boc-PEG47-NH₂ 2 (269 mg, 0.1170 mmol), TBTU (45.1 mg, 0.1404 mmol), and DIPEA (60 uL). After stirring the resulting suspension overnight, water was added. The mixture was extracted using DCM:20% TFE and the combined organic phases were dried over Na₂SO₄. After filtration, the solvent was removed under vacuum to dryness and the crude compound 3 was purified by flash chromatography (DCM:20% MeOH). To the product was added 2 mL of 4N HCl:Dioxane under anhydrous conditions until the deprotection was determined to be complete by LC-MS: calculated [M+H]+ for 2483.59 m/z, found 2484.01.

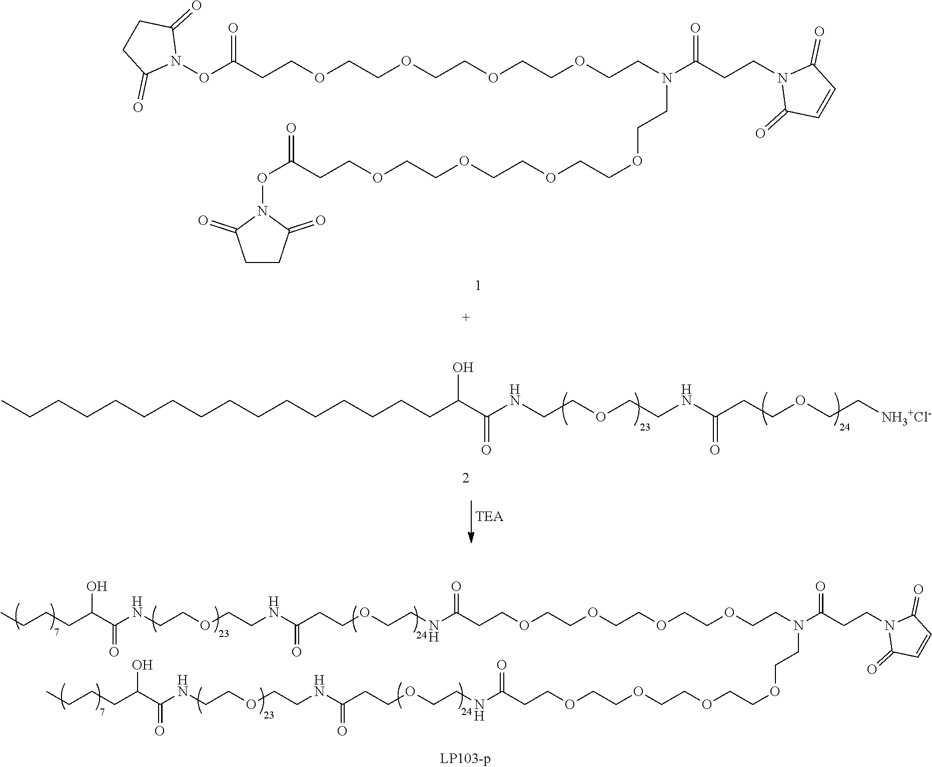

To a solution of compound 4 (10 mg, 0.0116 mmol, 1.0 equiv.) and compound 5 (59.3 mg, 0.0239 mmol, 2.05 equiv.) in anhydrous DCM (2 mL) was added triethylamine (8 uL, 0.0582 mmol, 5.0 equiv.) at room temperature. The reaction mixture was kept at room temperature overnight and the solvent was removed under vacuum. LP103-p was separated by CombiFlash® eluting with 10-17% methanol in dichloromethane. LC-MS: calculated [M+6H]+/6 933, found 934, calculated [M+7H]+/7 800, found 801.

### Synthesis of LP104-p

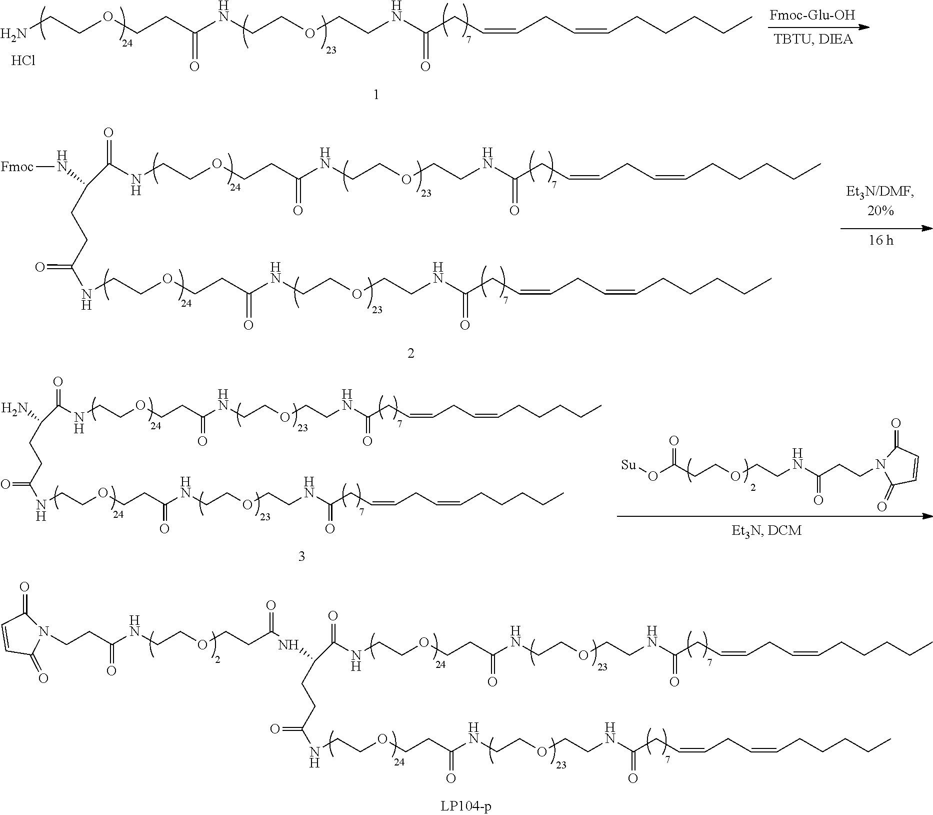

Compound 1 (synthesis shown in procedures for LP87, above), was conjugated with Fmoc-Glu-OH as described in the procedure for LP54-p, above. Calculated MW 5261.56, (M+3×18)/3=1771.86, (M+4×18)/4=1333.39 Found: MS (ES, pos): 1771.98 [M+3NH₄]³⁺, 1333.57 [M+4NH₄]⁴⁺.

Compound 2 was Fmoc-deprotected as described for compound 11 in the synthesis of LP39-p, above. The resulting product 3 was conjugated with activated ester compound 4 as described in the procedure for synthesizing LP39-p, above. LP104-p was isolated following CombiFlash® purification. Calculated MW 5349.62, (M+3×18)/3=1801.21, (M+4×18)/4=1355.41. Found: MS (ES, pos): 1801.87 [M+3NH₄]³⁺, 1355.92 [M+4NH₄]⁴⁺.

### Synthesis of LP106-p

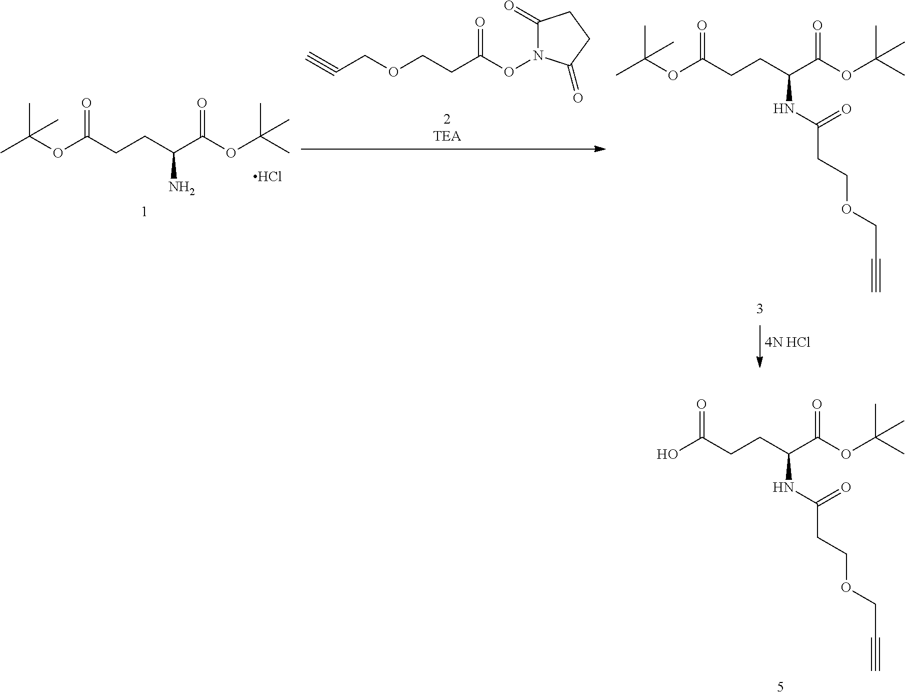

To compound 1 (200 mg, 0.676 mmol) in DCM (4 mL) was added TEA (218 uL, 1.56 mmol) then compound 2 (198 mg, 0.879 mmol) and the mixture was stirred at room temperature for 1 hour. Upon completion all volatiles were removed and crude compound 3 was deprotected using 4N HCl to provide acid 5 which was used subsequently without further purification.

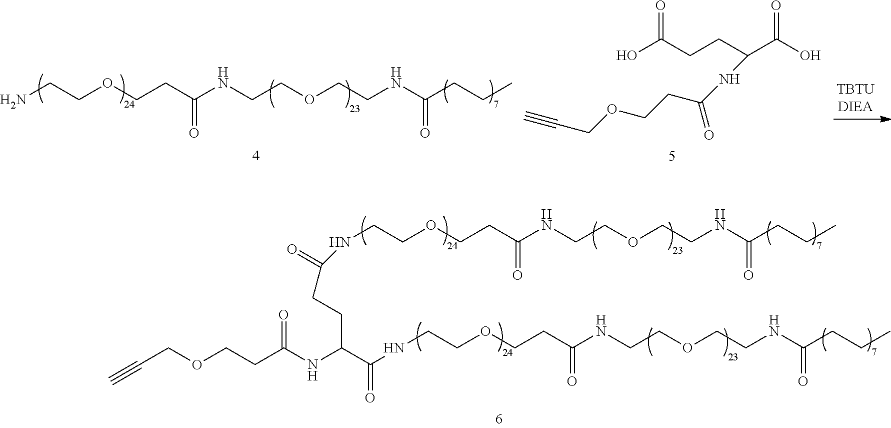

Crude compound 5 (60 mg, 0.1014 mmol assumed) was dissolved in DMF (1 mL), treated with TBTU (71.6 mg, 0.223 mmol) and stirred for 5 minutes. Compound 4 (668 mg, 0.273 mmol) and DIEA (91.8 uL, 0.527 mmol) in DMF (1 mL) were subsequently added and the mixture was left to stir at room temperature for 16 hours. Upon completion all volatiles were removed and compound 6 was isolated eluting a gradient of MeOH (0.1% TFA) in water (0.1% TFA) using a Waters™ Phenomenex C18 Gemini column (10 u, 50 mm×250 mm).

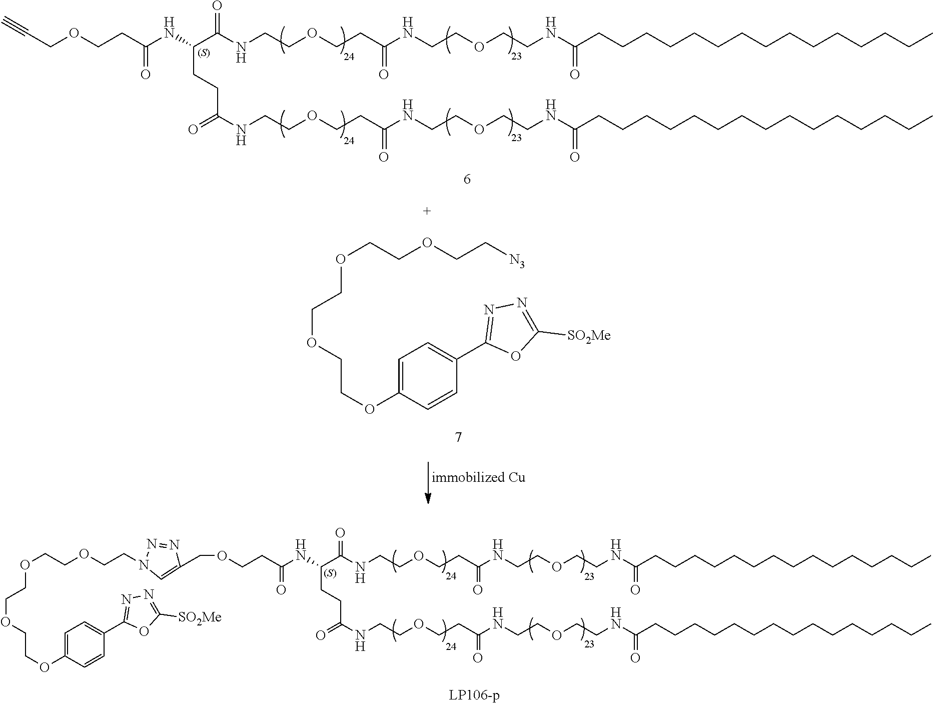

Compound 6 (23.5 mg, 0.0532 mmol) and Compound 7 (29.9 mg, 0.0586 mmol) were dissolved in 12.0 mL DMF and the vessel was sparged with N₂ for 5 minutes. Then, immobilized copper (337 mg, 0.0532 mmol) and sodium ascorbate (31.6 mg, 0.1597 mmol) were added and the reaction mixture was stirred at 40° C. overnight.

The resin and other solids were filtered off. The filtrate was concentrated under vacuum and purified by HPLC to yield LP106-p.

### Synthesis of LP107-p

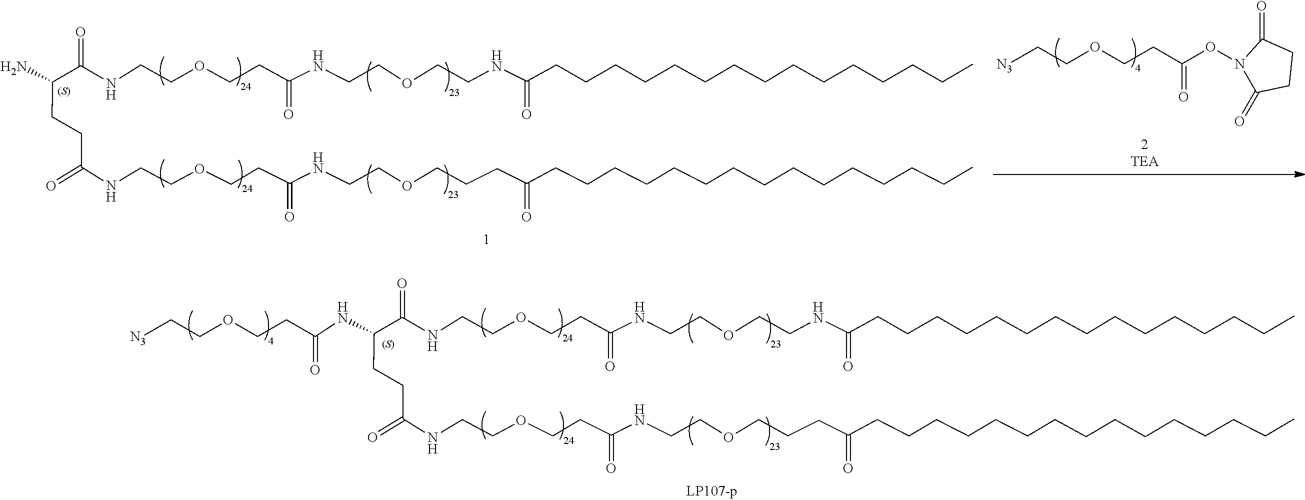

Compound 1 (982 mg, synthesized as shown in the procedures for LP38-p, above) was dissolved in 10 mL DCM. Compound 2 (90 mg) and triethylamine (0.081 mL) were added. The reaction mixture was stirred at room temperature for 5-8 hours until completion. The product was extracted using 1N HCl, followed by sat. NaHCO₃ then washed brine, and finally dried with Na₂SO₄. LP107-p was further purified using column chromatography.

### Synthesis of LP108-p

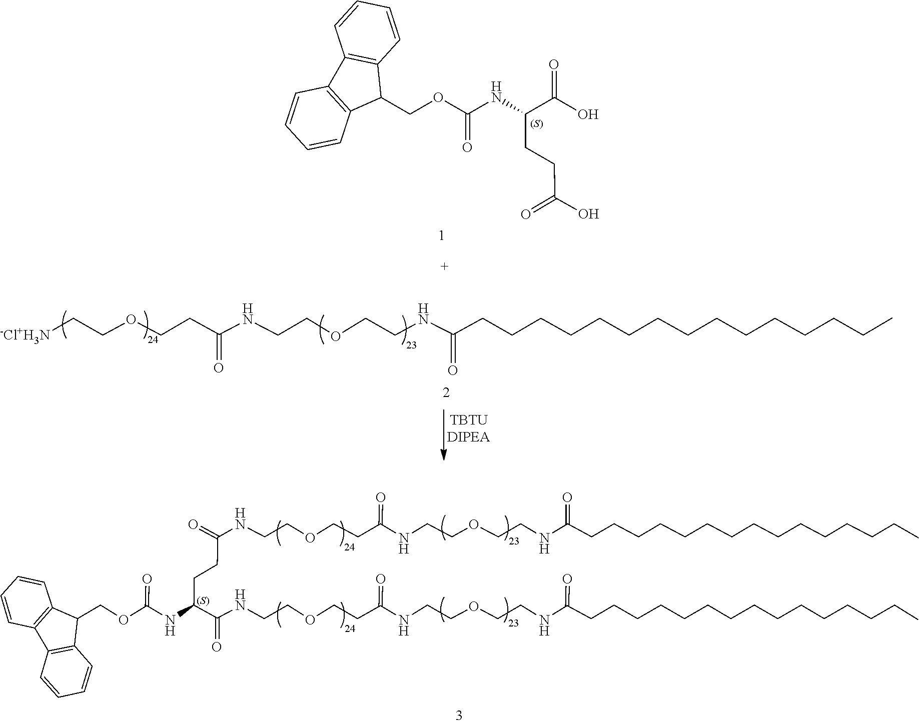

To a solution of compound 1 (595 mg, 1.610 mmol, 1.0 equiv.), compound 2 (8377 mg, 3.382 mmol, 2.10 equiv.), and diisopropylethylamine (1.122 mL, 6.443 mmol, 4.0 equiv.) in anhydrous DMF (100 mL) was added TBTU (1241 mg, 3.865 mmol, 2.4 equiv.) at room temperature. The reaction mixture was kept at room temperature for 3 hours. The reaction mixture was then concentrated. The residue was washed with saturated ammonium chloride and sodium bicarbonate aqueous solution. Compound 3 was purified by CombiFlash® eluting with 12-20% methanol in dichloromethane. LC-MS: [M+5H]/5, calculated 1043.05, found 1044.38.

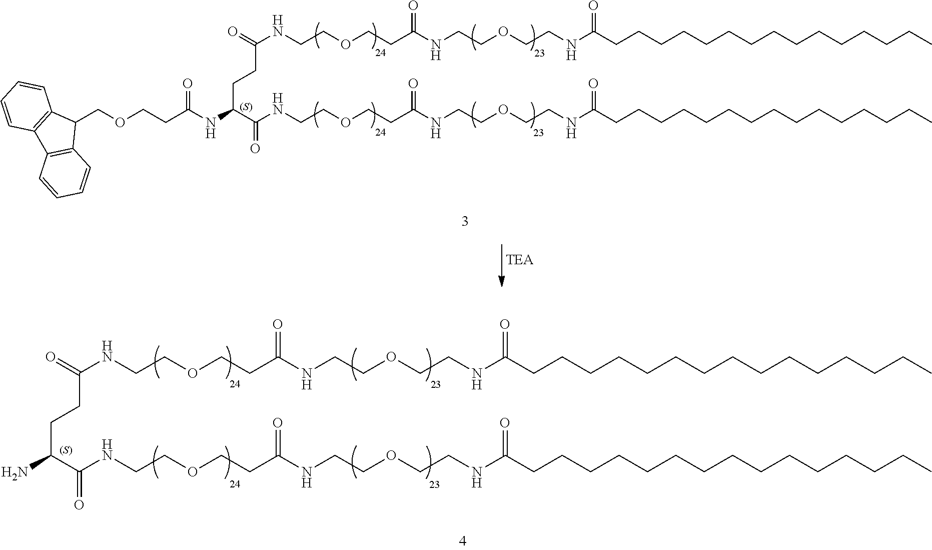

To a solution of compound 1 (104 mg, 0.0199 mmol, 1.0 equiv.) in anhydrous DMF (1.6 mL) was added TEA (0.4 mL) at room temperature. The reaction mixture was kept at room temperature overnight and the solvent was removed under vacuum. Compound 4 was used directly without further purification. LC-MS: [M+5H]/5 calculated 998.63, found 999.97.

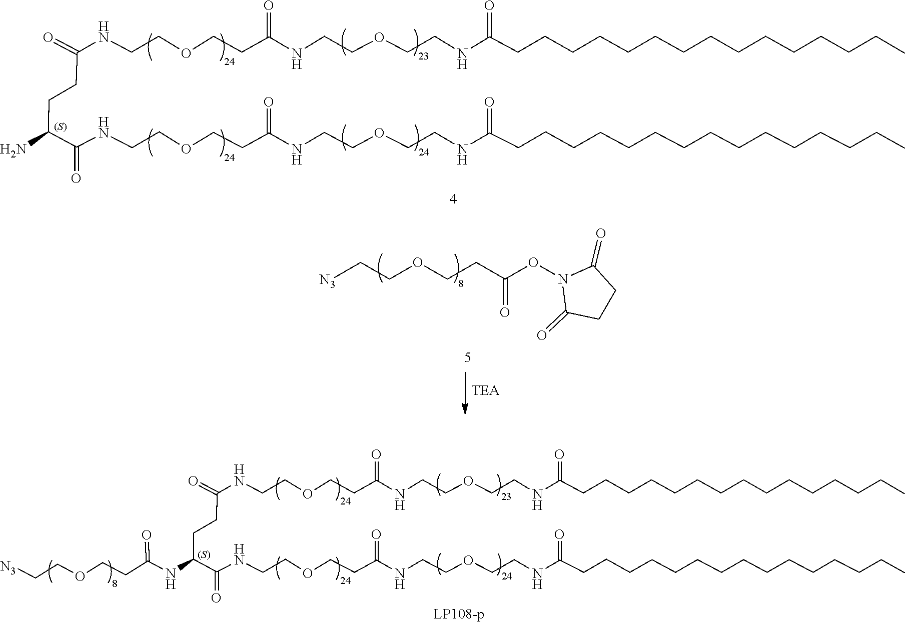

To a solution of compound 4 (99 mg, 0.198 mmol, 1.0 equiv.) and compound 5 (134 mg, 0.238 mmol, 1.2 equiv.) in anhydrous DCM (3 mL) was added triethylamine (0.006 mL, 0.0397 mmol, 2.0 equiv.) at room temperature. The reaction mixture was kept at room temperature overnight and the solvent was removed under vacuum. LP108-p was separated by CombiFlash® eluting with 12-20% methanol in dichloromethane. LC-MS: calculated [M+5H]/5 1088.48, found 1089.86.

### Synthesis of LP109-p

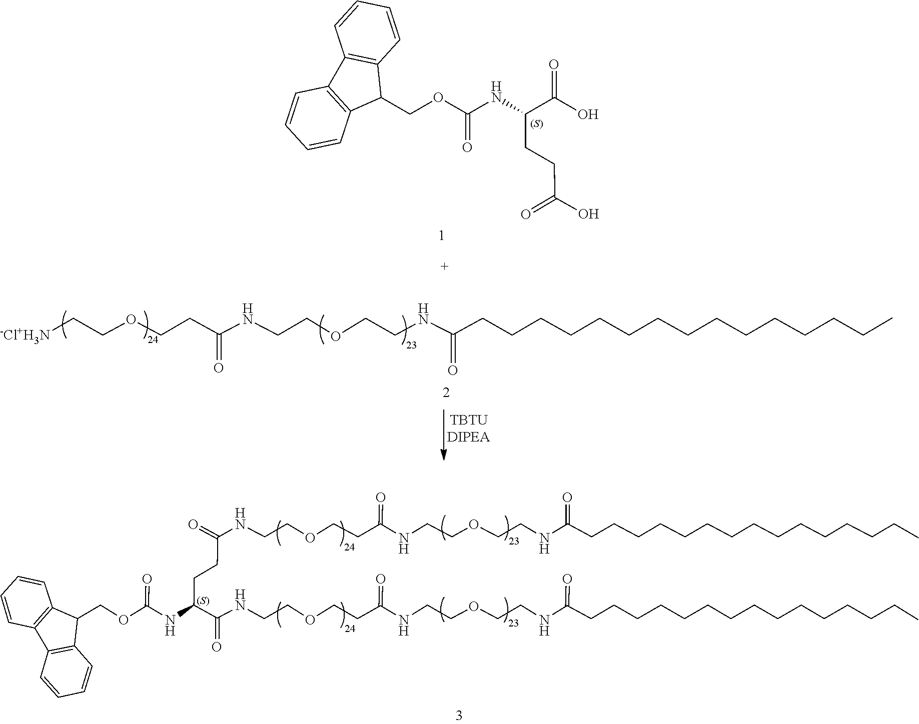

To a solution of compound 1 (595 mg, 1.610 mmol, 1.0 equiv.), compound 2 (8377 mg, 3.382 mmol, 2.10 equiv.) and diisopropylethylamine (1.122 mL, 6.443 mmol, 4.0 equiv.) in anhydrous DMF (100 mL) was added TBTU (1241 mg, 3.865 mmol, 2.4 equiv.) at room temperature. The reaction mixture was kept at room temperature for 3 hours. The reaction mixture was then concentrated. The residue was washed with saturated ammonium chloride and sodium bicarbonate aqueous solution. Compound 3 was purified by CombiFlash® eluting with 12-20% methanol in dichloromethane. LC-MS: [M+5H]/5, calculated 1043.05, found 1044.38.

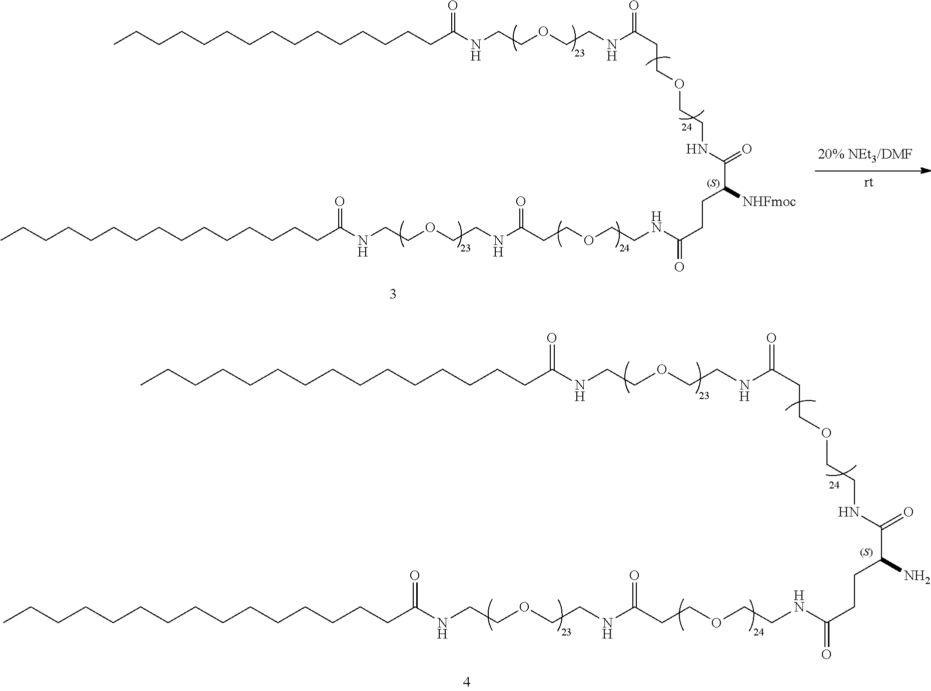

To compound 3 (100 mg) was added 20% NEt₃ (0.053 mL) in DMF at room temperature. The reaction mixture was stirred at room temperature until full conversion was confirmed via LC-MS. The reaction mixture was azeotroped with PhMe/MeOH and concentrated under high-vacuum overnight to obtain crude compound 4. LC-MS: calculated [M+H]+ 4989.17 m/z, observed 1262.31 (+4/4, +H₂O) m/z.

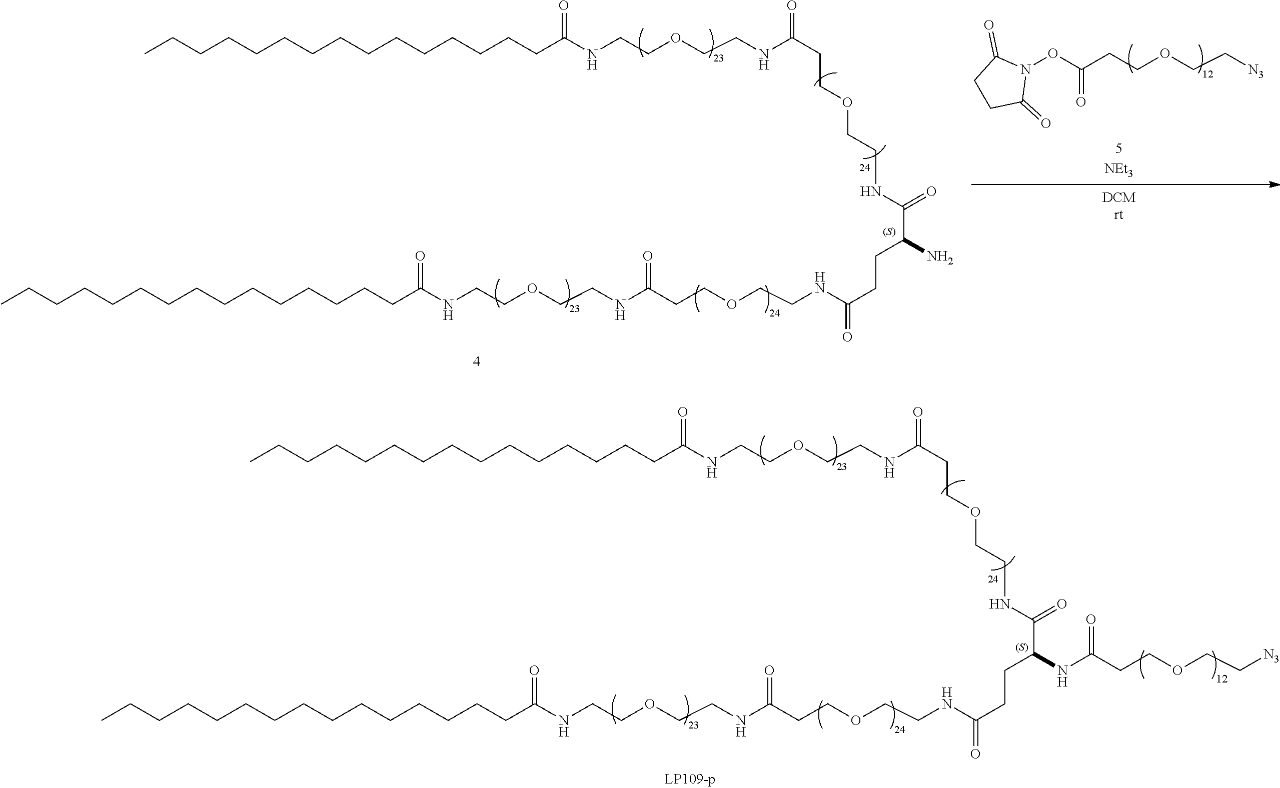

A solution of compound 4 (95.7 mg) and NEt₃ in anhydrous DCM (0.008 mL) under sparging N₂(g) was prepared at room temperature. Compound 5 (14.2 mg) was then added slowly. The reaction mixture was allowed to stir until full conversion was observed by LC-MS. The reaction mixture was then directly concentrated. The residue was purified by CombiFlash® via a 12-g column of silica gel as the stationary phase with a gradient of 0-20% MeOH in DCM (0% B to 100% B) over 20 minutes, in which LP109-p eluted at 100% B to provide clean and impure fractions. Two clean fractions were collected and concentrated. An impure fraction was concentrated and re-subjected to reaction conditions to push further conversion. Isolation via a gradient of 0-20% MeOH in DCM (0% B to 100% B) provided improved yet somewhat impure LP109-p elution at 88% B. LC-MS: calculated [M+H]+ 5614.51 m/z, observed 1422.64 (+4/4, +H₂O) m/z.

### Synthesis of LP110-p

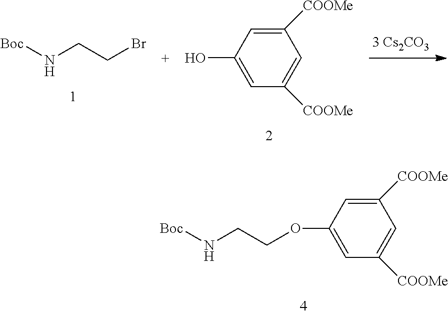 [SMILES: CC(C)(C)OC(=O)NCCBr ; COC(=O)c1cc(O)cc(C(=O)OC)c1 ; COC(=O)c1cc(OCCNC(=O)OC(C)(C)C)cc(C(=O)OC)c1]

To a solution of compound 1 (4.00 g) in 20 mL DMF was added compounds 2 (4.50 g) and 3 (11.6 g) at room temperature. The reaction mixture was stirred overnight. The product was extracted by standard work up (1N NaOH, brine) and dried with Na₂SO₄. TLC showed that compound 2 was removed by NaOH. Compound 4 was used directly in the next step.

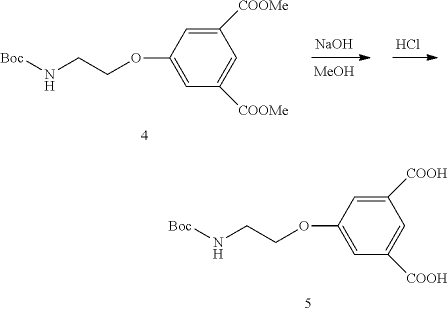 [SMILES: "MeOH" ; COC(=O)c1cc(OCCNC(=O)OC(C)(C)C)cc(C(=O)OC)c1 ; "NaOH" ; Cl ; CC(C)(C)OC(=O)NCCOc1cc(C(=O)O)cc(C(=O)O)c1]

To a solution of compound 4 (3.04 g) in 100 mL MeOH was added NaOH (1.03 g) solution at room temperature. The reaction mixture was stirred overnight. The reaction mixture was concentrated to remove MeOH. The aqueous phase was extracted with ethyl acetate to remove any unreacted starting material. The mixture was acidified to pH of 3, then extracted with ethyl acetate, dried using Na₂SO₄, and concentrated to produce compound 5 as a white solid. Compound 5 was used directly in the next step.

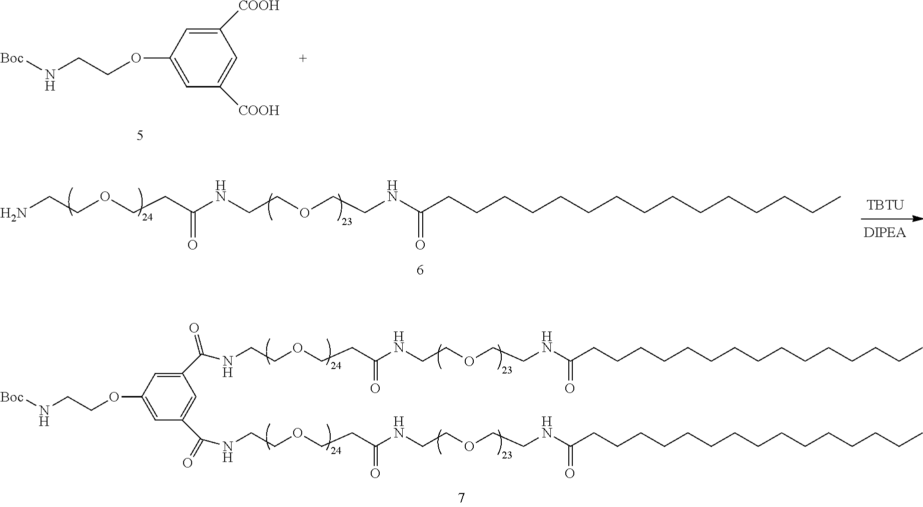

To compound 1 (2.9 mg) in DCM was added 2 equivalents of DIPEA (0.006 mL) at room temperature. Compound 6 (45 mg), TBTU (6.3 mg), and 2 equivalents of DIPEA (0.006 mL) was stirred at room temperature for 30 minutes. Slow addition of the activated acid mixture to PEG solution was achieved using a syringe pump (in 2-3 hours). The reaction mixture was stirred at room temperature. until full conversion was observed by TLC.

The product was extracted using a standard work up (1N HCl, sat. NaHCO₃, brine). The residue was purified by CombiFlash® using silica gel as the stationary phase with a gradient of 0-20% MeOH in DCM (0-100% B).

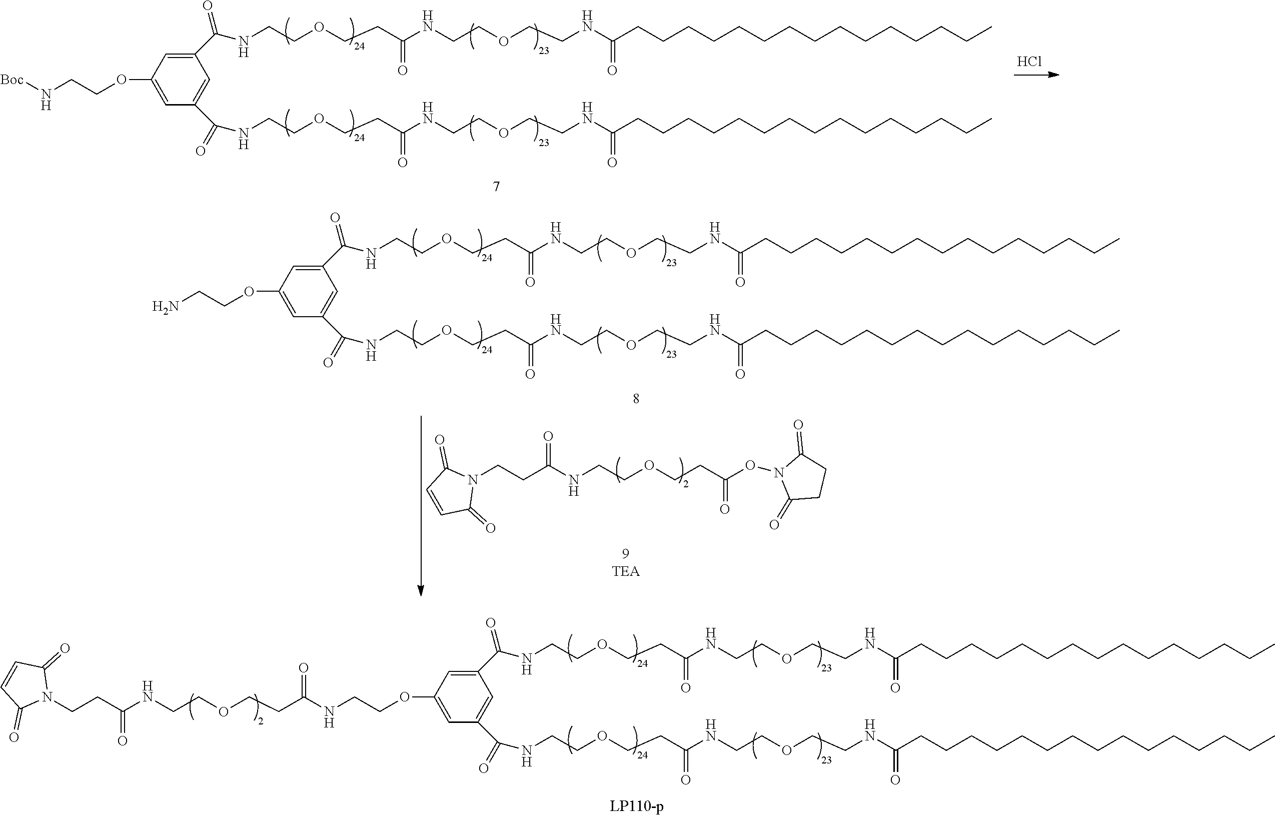

To compound 7 (27 mg) was added 1.5 mL 4 M HCl/dioxane at room temperature. The reaction mixture was stirred at room temperature for 1.5 h until full conversion was confirmed via LC-MS. The reaction mixture was concentrated under vacuum. Crude compound 8 was dissolved in DCM, and compound 9 (2.7 mg) and TEA (1.1 mg) were added. The reaction mixture was stirred at room temperature until full conversion was observed by TLC.

LP110-p was purified by CombiFlash® using silica gel as the stationary phase with a gradient of DCM to 20% MeOH in DCM (0-100% B).

### Synthesis of LP111-p

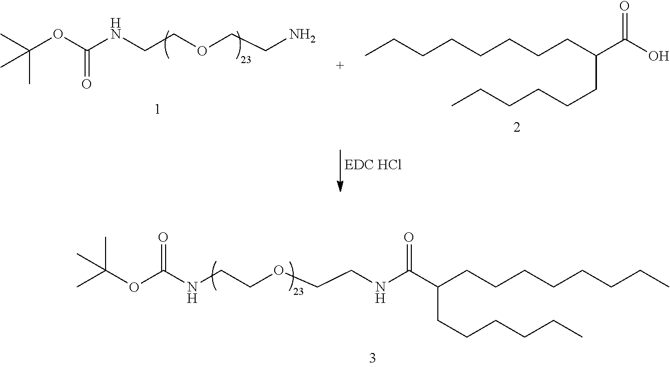

To a solution of compound 1 (2500 mg, 2.130 mmol, 1.0 equiv.) and compound 2 (655 mg, 2.556 mmol, 1.2 equiv.) in anhydrous DCM (10 mL) was added EDC HCl (630 mg, 3.195 mmol, 1.5 equiv.) at room temperature. The reaction mixture was kept at room temperature overnight. The reaction mixture was concentrated. Compound 3 was purified by CombiFlash® eluting with 8-18% methanol in dichloromethane. LC-MS: calculated [M+H]+ 1411.95, found 1412.80.

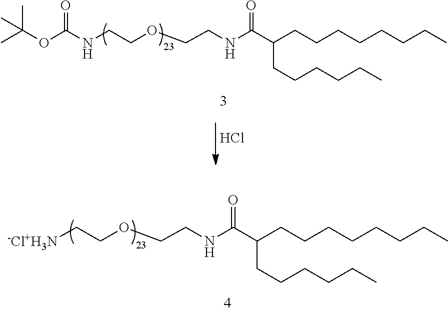

To compound 3 (2400 mg, 1.699 mmol, 1.0 equiv.) was added 4M HCl in dioxane (8.499 mL, 33.997 mmol, 20 equiv.) at room temperature. The reaction mixture was kept at room temperature for 1 hour and was then concentrated. Compound 4 was used directly without further purification. LC-MS: [M+H]/+ calculated 1311.90, found 1312.95.

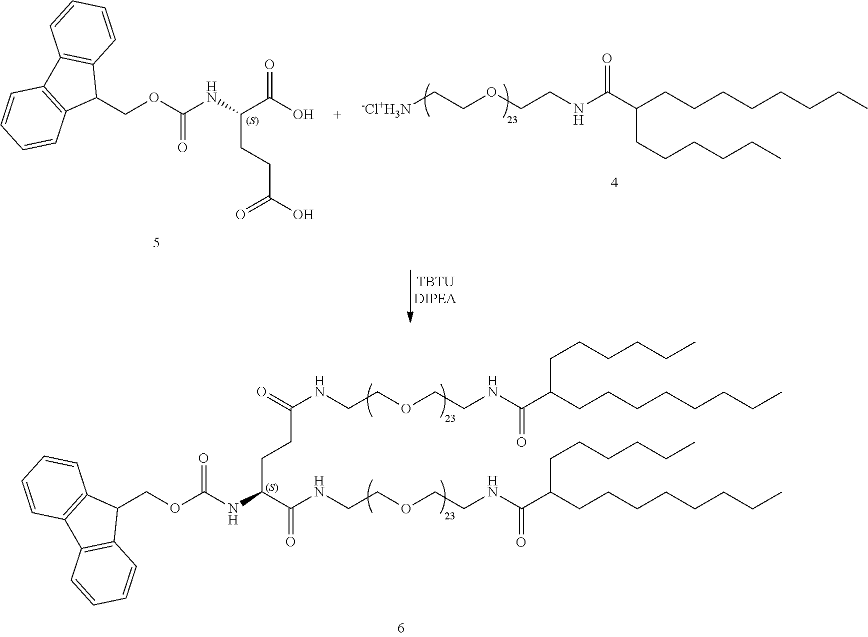

To a solution of compound 5 (300 mg, 0.812 mmol, 1.0 equiv.), compound 4 (2.299 g, 1.705 mmol, 2.10 equiv.), and diisopropylethylamine (0.566 mL, 3.248 mmol, 4.0 equiv.) in anhydrous DMF (10 mL) was added TBTU (625 mg, 1.949 mmol, 2.4 equiv.) at room temperature. The reaction mixture was kept at room temperature for 1 hour. The reaction mixture was then concentrated. The residue was washed with saturated ammonium chloride and sodium bicarbonate aqueous solution. Compound 6 was purified by CombiFlash® eluting with 10-18% methanol in dichloromethane. LC-MS: [M+2H]/2, calculated 1478.45, found 1479.89.

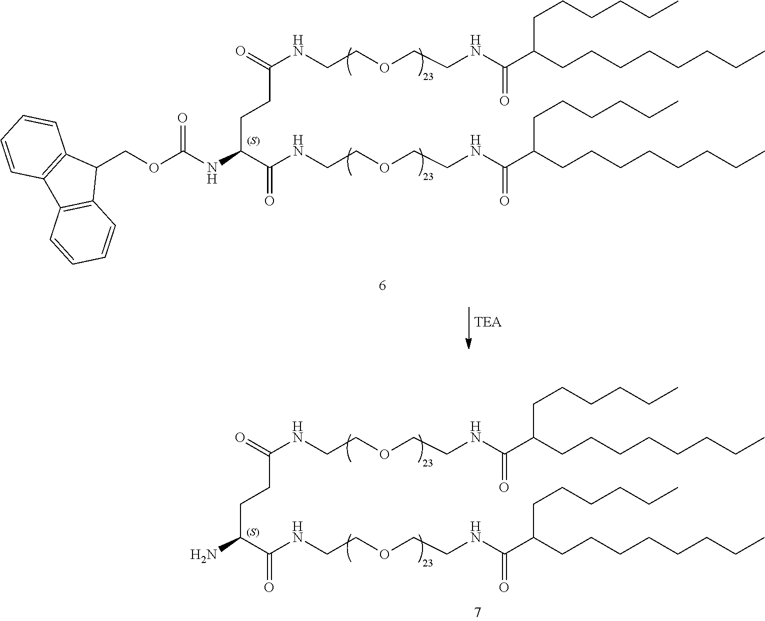

To a solution of compound 6 (1690 mg, 0.571 mmol, 1.0 equiv.) in anhydrous DMF (8 mL) was added triethylamine (2 mL) at room temperature. The reaction mixture was kept at room temperature overnight and the solvent was removed under vacuum. Compound 7 was used directly without further purification. LC-MS: [M+2H]/2 calculated 1367.41, found 1368.88.

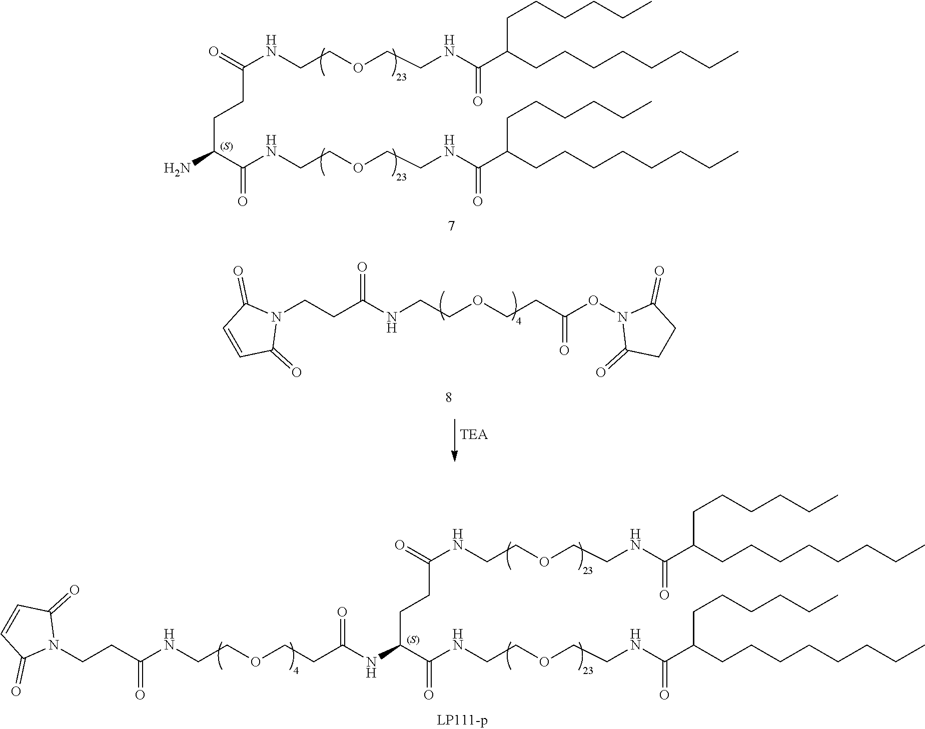

To a solution of compound 7 (1563 mg, 0.571 mmol, 1.0 equiv.) and compound 2 (381 mg, 0.743 mmol, 1.3 equiv.) in anhydrous DCM (10 mL) was added TEA (0.162 mL, 1.143 mmol, 2.0 equiv.) at room temperature. The reaction mixture was kept at room temperature overnight and the solvent was removed under vacuum. LP111-p was purified by CombiFlash® eluting with 8-16% methanol in dichloromethane. LC-MS: calculated [M+3H]/3 1044.67, found 1046.18.

### Synthesis of LP124-p

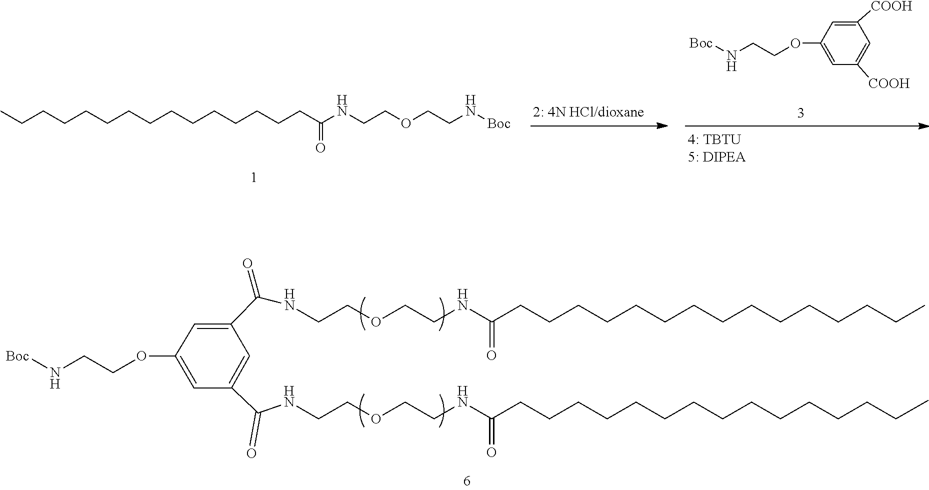

To compound 1 (760 mg) was added 2 mL of 4 M HCl/dioxane at room temperature. The reaction mixture was stirred at room temperature. The reaction mixture was stirred for 1.5 hours until full conversion was confirmed via LC-MS. The reaction mixture was concentrated under vacuum. The residue was dissolved in DCM, and compounds 3 (84.1 mg), 4 (207 mg), and 5 (0.281 mL) were added. The reaction mixture was stirred at room temperature until full conversion was observed by TLC.

The product was extracted by standard work up (1N HCl, sat. NaHCO₃, brine). Compound 6 was purified by CombiFlash® using silica gel as the stationary phase with a gradient of 0-20% MeOH in DCM (0-100% B).

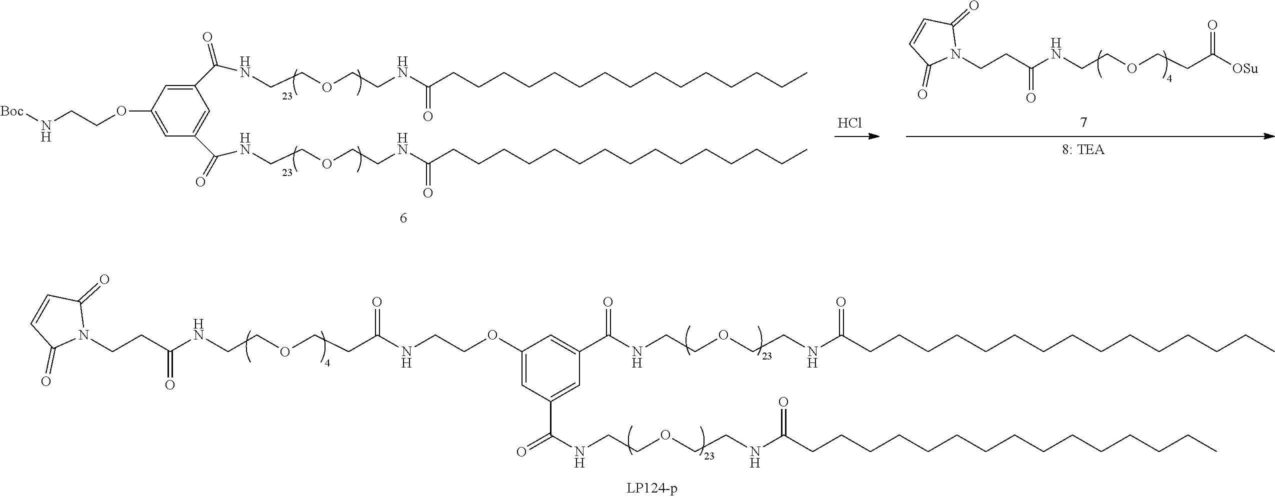

To compound 6 (250 mg) was added 4 mL 4 M HCl/dioxane at room temperature. The reaction mixture was stirred at room temperature for 2 hours until full conversion was confirmed via LC-MS. The reaction mixture was concentrated under vacuum. The residue was dissolved in DCM, then compounds 7 (52.9 mg) and 8 (0.036 mL) were added. The reaction mixture was stirred at room temperature until full conversion was observed by TLC.

LP124-p was purified by CombiFlash® using silica gel as the stationary phase with a gradient of 0-20% MeOH in DCM (0-100% B).

### Synthesis of LP130-p

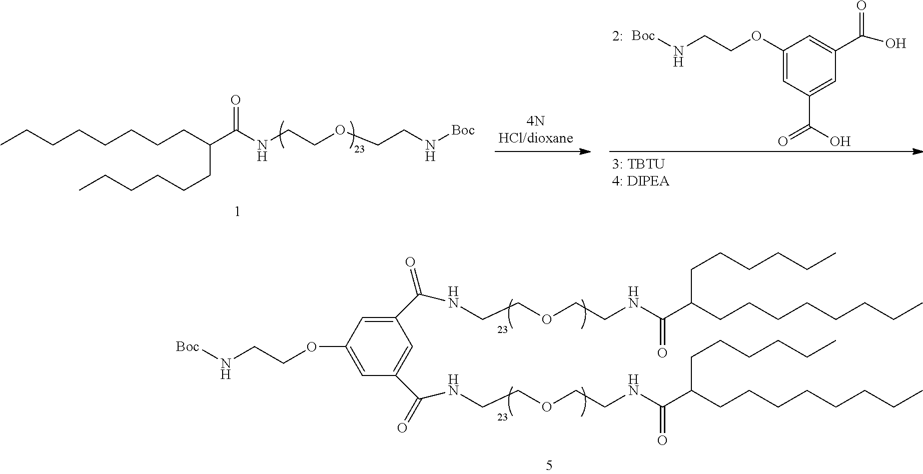

To compound 1 (1.89 g) was added 5 mL of 4 M HCl/dioxane at room temperature. The reaction mixture was stirred at room temperature for 1.5 hours until full conversion was confirmed via LC-MS. The reaction mixture was then concentrated under vacuum. The residue was dissolved in DCM, and compounds 2 (209 mg), 3 (516 mg) and 4 (0.70 mL) were added. The reaction mixture was stirred at room temperature until full conversion was observed by TLC.

The product was extracted by a standard work up (1N HCl, sat. NaHCO₃, brine). Compound 5 was purified by CombiFlash® using silica gel as the stationary phase with a gradient of 0-20% MeOH in DCM (0-100% B).

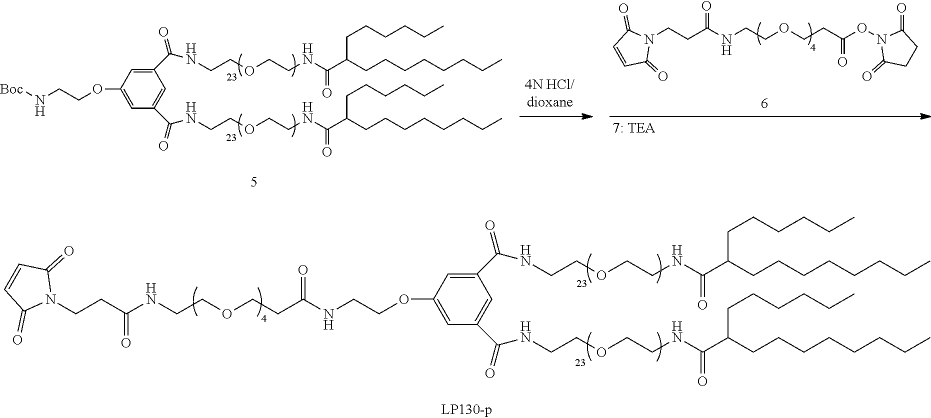

To compound 5 (800 mg) was added 5 mL of 4 N HCl/dioxane at room temperature. The reaction mixture was stirred at room temperature for 2 hours until full conversion was confirmed via LC-MS. The reaction mixture was then concentrated under vacuum. The residue was dissolved in DCM, then compounds 2 (169 mg) and 3 (0.116 mL) were added. The reaction mixture was stirred at room temperature until full conversion was observed by TLC.

LP130-p was purified by CombiFlash® using silica gel as the stationary phase with a gradient of DCM to 20% MeOH in DCM (0-100% B).

### Synthesis of LP143-p

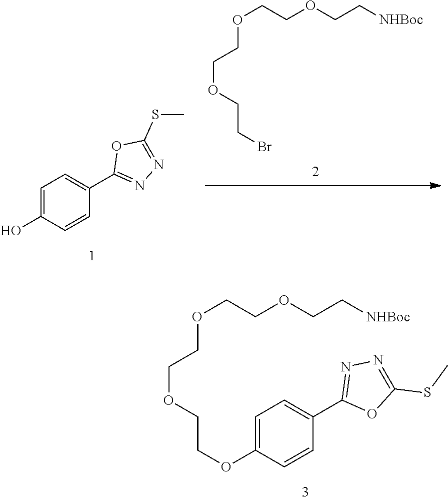

Compound 1 (500 mg) was dissolved in 10 mL anhydrous THF in a pressure vessel and K₂CO₃ (398 mg) was added. Compound 2 (983 mg) was added as a solution in a minimal amount of DMF and the vessel was capped and the reaction mixture was set to stir overnight at 40° C. Then, the reaction mixture was allowed to cool to room temperature. The solids were filtered off and the reaction mixture was concentrated under vacuum. Compound 3 was a purified using flash chromatography eluting with 0-100% EtOAc in hexanes.

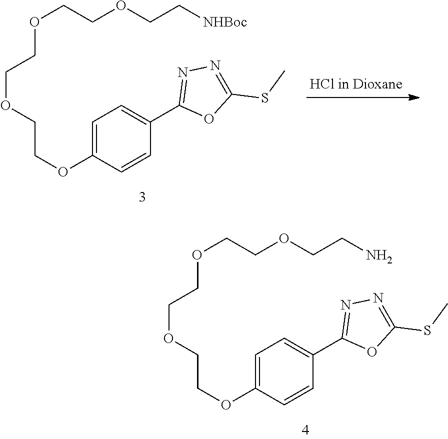 [SMILES: CSc1nnc(-c2ccc(OCCOCCOCCOCCNC(=O)OC(C)(C)C)cc2)o1 ; CSc1nnc(-c2ccc(OCCOCCOCCOCCN)cc2)o1]

Compound 3 (1070 mg) was dissolved in 4 mL of 4 M HCl in dioxanes and stirred until all Boc was removed. The reaction mixture was then concentrated. Compound 4 was purified using flash chromatography eluting with 0-20% MeOH in DCM.

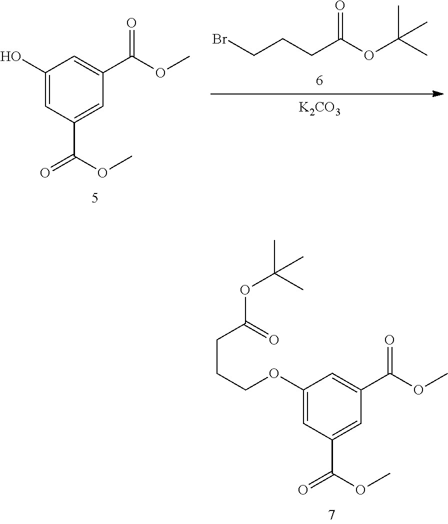

Compound 5 (1000 mg) was dissolved in 5 mL anhydrous DMF in a pressure vessel and K₂CO₃ (1.315 g) was added. Then, compound 6 (850 mg) was added in a minimal amount of DMF and the reaction mixture was capped and stirred at 40° C. Then, the reaction mixture was allowed to cool to room temperature. The solids were filtered off and then the reaction mixture was concentrated under vacuum. Compound 7 was purified using flash chromatography eluting with 0-100% EtOAc in hexanes.

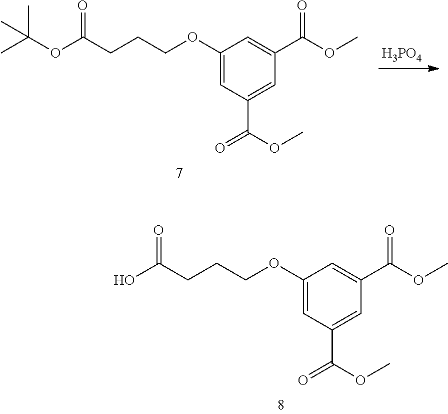

H₃PO₄ (0.594 mL) was added to a stirred solution of compound 7 (900 mg) in 20 mL of toluene. The reaction mixture was stirred overnight at room temperature. The reaction mixture was then diluted with water (30 mL) and washed 3 times with ethyl acetate (30 mL). The combined organic layers were dried over sodium sulfate and concentrated.

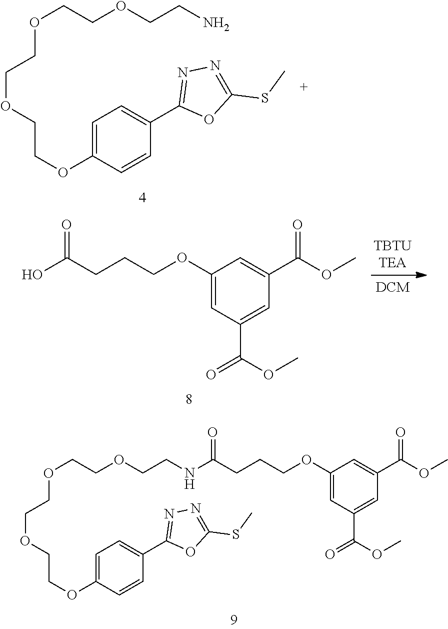

Compound 8 (100 mg) and TBTU (149 mg) were dissolved in 2 mL DMF and were stirred for 5 minutes. Then, TEA (0.152 mL) and compound 4 (142 mg) were added to the mixture and the reaction mixture was stirred at room temperature overnight. The reaction mixture was diluted with ethyl acetate (10 mL) and washed with saturated ammonium chloride (3×10 mL). The organic layer was dried over sodium sulfate and concentrated. Compound 9 was purified using flash chromatography eluting with 0-100% hexanes-ethyl acetate and then DCM/MeOH 0-20%.

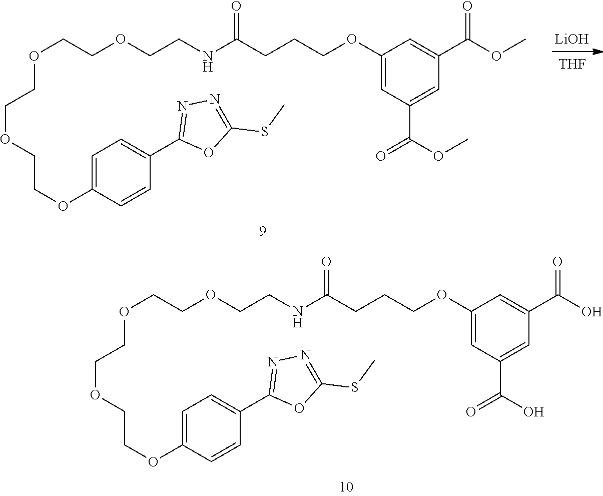

Compound 9 (197 mg) was dissolved in 4 mL THF. Then, LiOH (43 mg) and water (0.4 mL) were added. The reaction mixture was stirred until deprotection was confirmed by LC-MS. The reaction mixture was quenched with Amberlyst® 15. The Amberlyst was filtered off and the reaction mixture was concentrated. Compound 10 was purified using flash chromatography eluting with 0-100% ethyl acetate in hexanes with 0.1% HOAc additive.

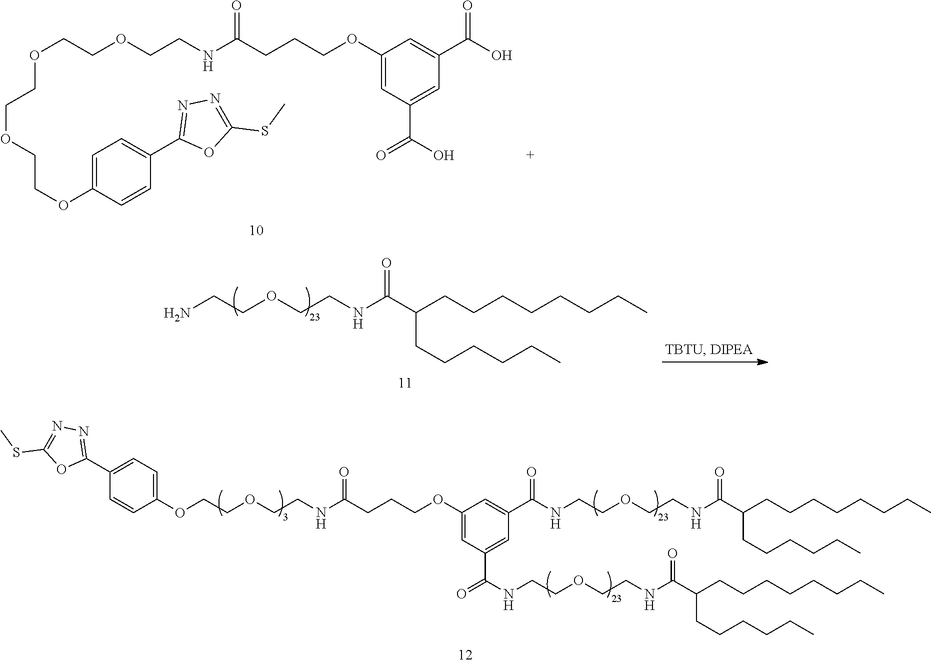

Compound 10 (380 mg) was mixed with TBTU (424 mg) in 4 mL DMF for five minutes. Then, compound 11 (2.12 g) was added, followed by DIPEA (0.542 mL). The reaction mixture was stirred at room temperature and kickers were added as follows: 50% TBTU and 50% DIPEA at 2 hours, 25% TBTU and 50% DIPEA at 3 hours, 50% DIPEA at 4 hours, 50% DIPEA at 5 hours. The reaction mixture was quenched after 6.5 hours. The reaction mixture was diluted with 20% TFE in DCM (15 mL) and washed with saturated ammonium chloride two times (15 mL). The organic layer was dried over sodium sulfate and concentrated. Compound 12 was then purified by HPLC.

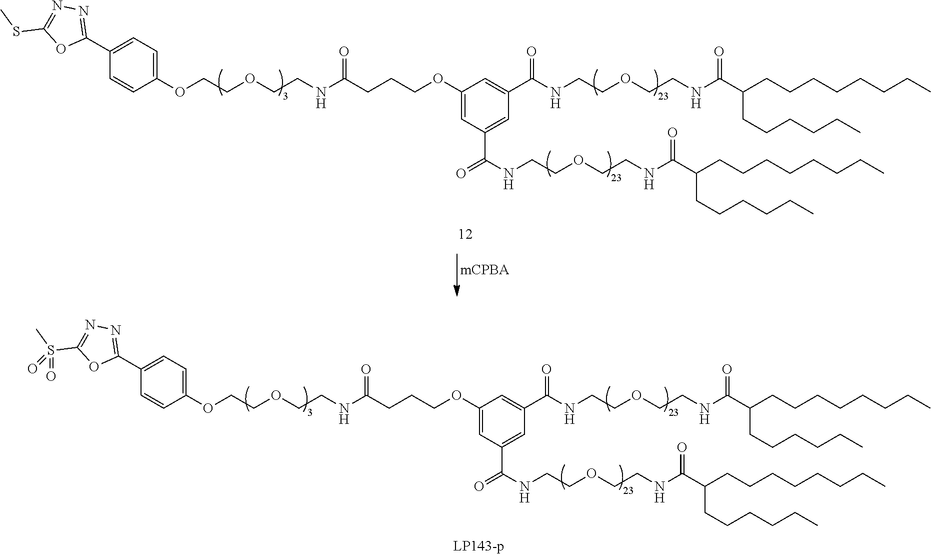

mCPBA (70% pure, 12 mg) was added to a stirring solution of compound 12 (28 mg) in 1 mL DCM at 0° C. The reaction mixture was allowed to warm up to room temperature stirring overnight and monitored via LCMS. The mixture was diluted with 20% TFE in DCM (5 mL), then washed with saturated sodium sulfite (2×5 mL) and once with saturated sodium bicarbonate (5 mL). The organic layer was dried over sodium sulfate. The correct mass was of LP143-p confirmed by LC-MS.

### Synthesis of LP210-p

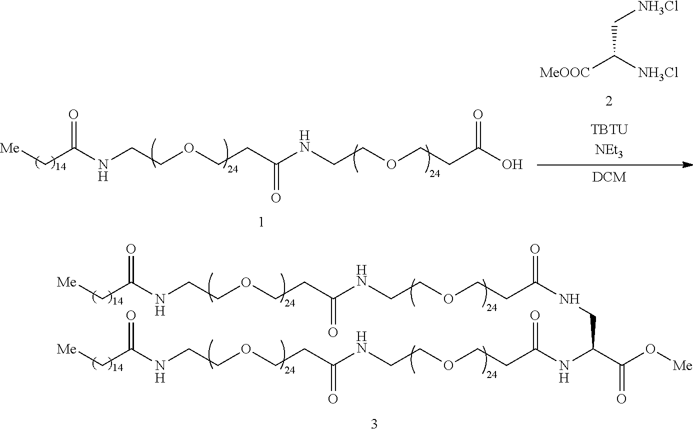

Compound 1 (0.2 g, 0.08 mmol) and TBTU (0.0542 g, 0.735 mmol) were dissolved in DCM (5 mL) and NEt₃ (0.0244 mL, 0.175 mmol) was added. In a separate vial, compound 2 (0.007 g, 0.037 mmol) and NEt₃ (0.0244 mL, 0.175 mmol) were stirred together in DCM (1 mL). The resulting solutions were stirred for 10 minutes. After 10 minutes the solution of compound 2 was added to the solution of compound 1. The resulting mixture was stirred for 90 minutes and then checked by LC-MS. The reaction mixture was quenched with 5 mL of water and stirred for 5 minutes. The layers were separated, and the organic layer was washed with sat. NaHCO₃(aq) (2×20 mL), water (20 mL), sat. NH₄Cl(aq) (2×20 mL), sat. NaCl(aq) (2×20 mL), dried over Na₂SO₄ and concentrated to yield crude compound 3 as a waxy off white solid (ca. 200 mg). The crude product was purified by silica gel chromatography eluting with 0-20% MeOH in DCM. Pure fractions were combined to yield 50 (27% yield) of compound 3 as a white solid.

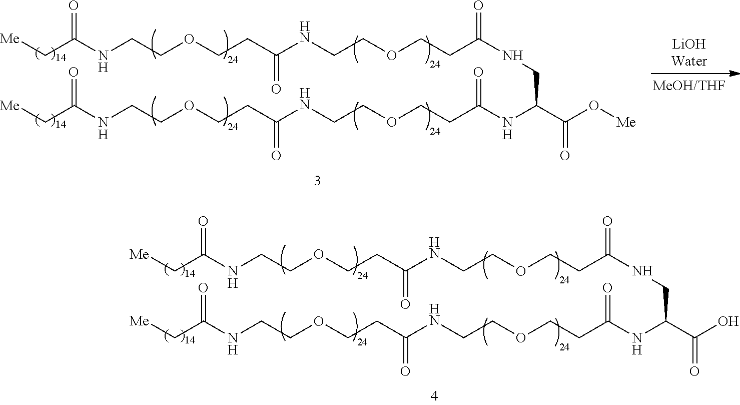

Compound 3 (0.05 g, 0.010 mmol) was dissolved in 1:1 MeOH/THF (5 mL), and LiOH (0.042 g, 1.74 mmol) and water (100 μL, 5.55 mmol) was added. The reaction mixture was stirred at room temperature overnight and checked by LC-MS. Organics were evaporated off and the resulting suspension was diluted with approximately 10 mL of water. The resulting suspension was acidified with 3 M HCl(aq) to a pH of 1 and was extracted with DCM (3×25 mL) The combined organics were washed with brine, dried over Na₂SO₄, concentrated, and dried under vacuum to yield 49 mg (98% yield) of compound 4 as an off white solid. The product was used without further purification.

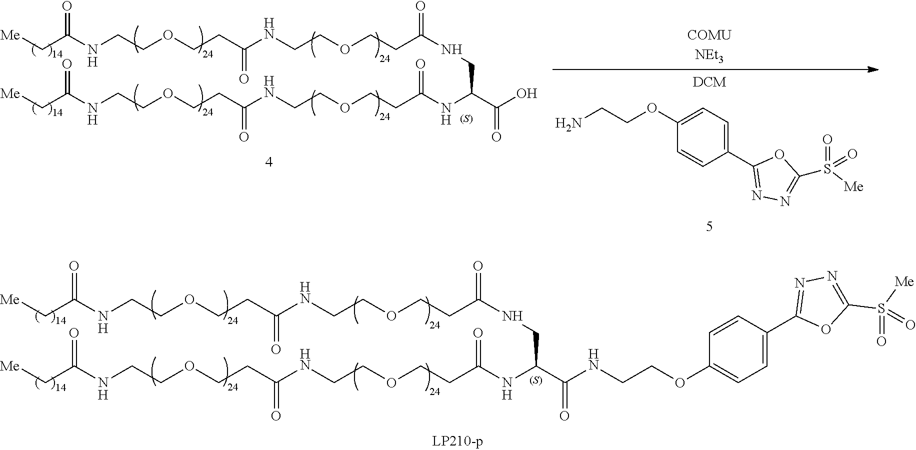

Compound 4 (0.05 g, 0.010 mmol) and COMU (0.0063 g, 0.015 mmol) were dissolved in DCM (1 mL) and NEt₃ (13.7 μL, 0.098 mmol) was added, the resulting solution was stirred for 10 minutes. In a separate vial Compound 5 was dissolved in DCM (0.3 mL). After 10 minutes the solution of compound 5 was added to the solution containing 1807-019. The resulting solution was stirred for 2 hrs. The reaction mixture was quenched with 1 M HCl(aq) (10 mL) and the organic layer was diluted with 10 mL DCM. The layers were separated, and the organic layer was further washed with 1M HCl(aq) (20 mL), sat. NHCO₃(aq) (1×20 mL) sat. NaCl(aq) (1×20 mL), dried over Na₂SO₄, concentrated, and dried under vacuum to yield 94 mg of crude LP210-p as an off white solid. The crude product was purified by silica gel chromatography eluting with 0-20% MeOH in DCM. Fractions containing pure LP210-p were combined and concentrated to yield 7 mg (13.3% yield).

### Synthesis of LP217-p

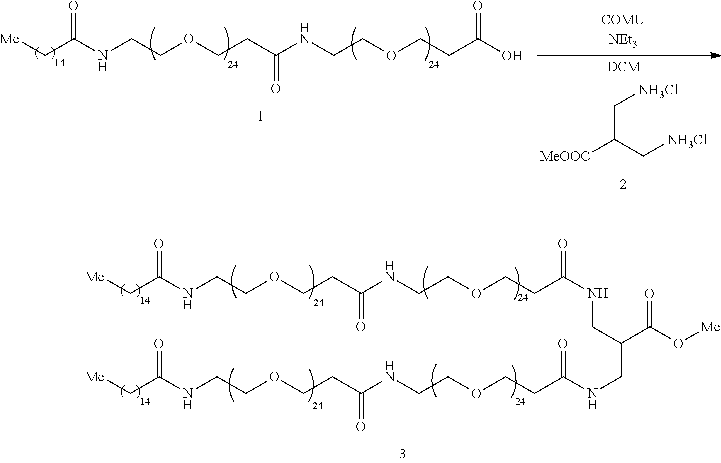

Compound 1 (0.265 g, 0.105 mmol) and COMU (0.0542 g, 0.735 mmol) were dissolved in DCM (5 mL) and NEt₃ (0.1 mL, 0.74 mmol) was added. The resulting solution was stirred for 10 minutes. After 10 minutes, compound 2 (0.010 g, 0.049 mmol) was added to the reaction. The resulting mixture was stirred overnight and checked by LC-MS. The reaction mixture was quenched with 5 mL of water and stirred for 5 minutes. The layers were separated, and the organic layer was washed with sat. NaHCO₃(aq) (2×20 mL), Water (20 mL), 2 M HCl(aq) (2×20 mL), sat. NaCl(aq) (20 mL), dried over Na₂SO₄, and concentrated to yield crude compound 3 as a waxy off white solid (ca. 350 mg). Crude compound 3 was purified by silica gel chromatography 2-20% MeOH in DCM. Fractions containing compound 3 were combined to yield 89 mg (36% yield) as an off white solid.

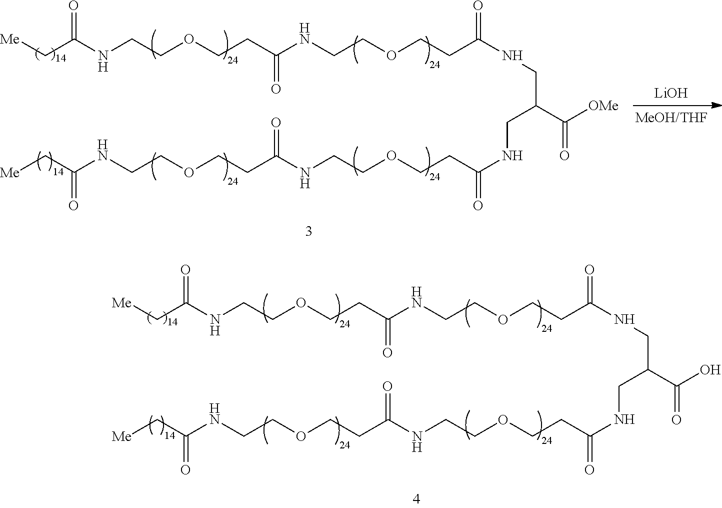

Compound 3 (0.089 g, 0.017 mmol) was dissolved in 1:1 MeOH/THF (5 mL) and LiOH (0.042 g, 1.74 mmol) and water (180 μL, 9.85 mmol) was added. The reaction mixture was stirred at room temperature overnight and checked by LC-MS. Organics were evaporated off and the resulting suspension was diluted with approximately 10 mL of water. The suspension was acidified with 3 M HCl(aq) to a pH of 1 and was extracted with DCM (3×25 mL). The combined organic layers were washed with brine, dried over Na₂SO₄, concentrated, and dried under vacuum to yield 81 mg (91% yield) of compound 4 as an off white solid. The product was used without further purification.

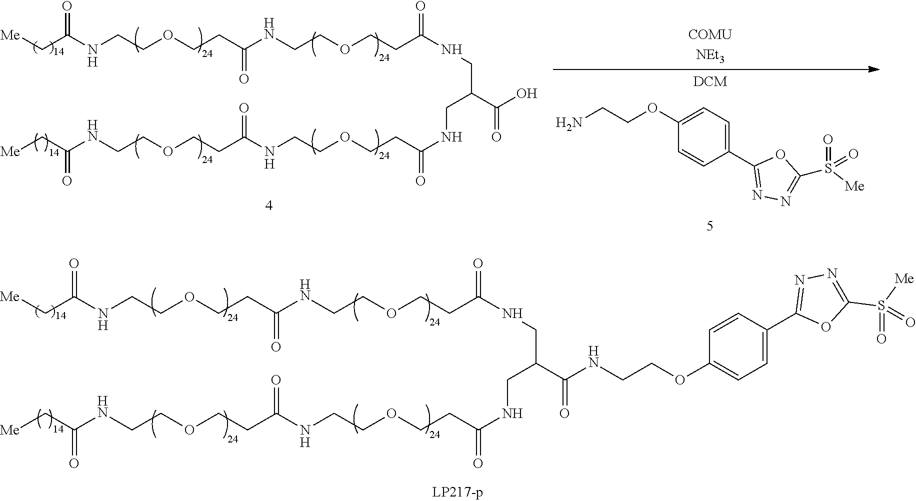

Compound 4 (0.081 g, 0.016 mmol) and COMU (0.010 g, 0.024 mmol) were dissolved in DCM (1 mL) and NEt₃ (44.2 μL, 0.32 mmol) was added. The resulting solution was stirred for 10 minutes. In a separate vial, compound 5 was dissolved in DCM (0.3 mL). After 10 minutes, the solution of compound 5 was added to the solution containing compound 4. The resulting mixture was stirred for 2 hours. The reaction mixture was quenched with 1 M HCl(aq) (10 mL) and the organic later was diluted with 10 mL DCM. The layers were separated, and the organic layer was further washed with 1M HCl(aq) (20 mL), sat. NHCO₃(aq) (1×20 mL) sat. NaCl(aq) (1×20 mL), dried over Na₂SO₄, concentrated, and dried under vacuum to yield 94 mg of crude LP217-p as an off white solid. The crude product was purified by silica gel chromatography 0-20% MeOH in DCM. Fractions containing pure LP217-p were combined and concentrated to yield 24 mg (28% yield).

### Synthesis of LP220-p

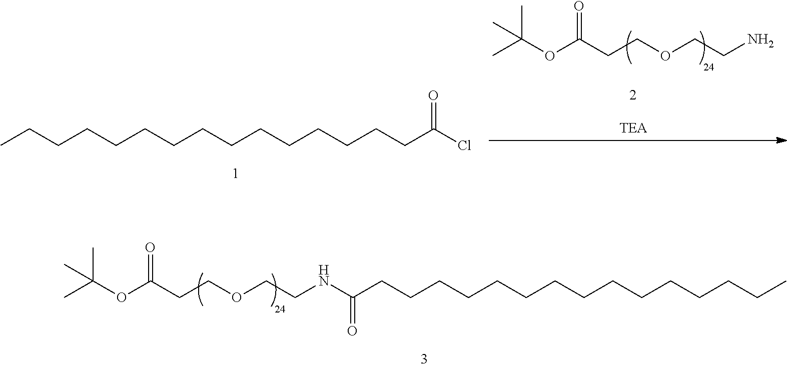

To a solution of compound 2 (3.3381 mmol, 4.0140 g) and TEA (4.0058 mmol, 0.4054 g, 0.558 mL) in DCM was added compound 1 (3.5050 mmol, 0.9634 g, 1.059 mL). The reaction mixture was stirred until full conversion of compound 2 was observed by LC-MS. The residue was purified by standard work up (1N HCl, sat. NaHCO₃, Brine wash, and dried over Na₂SO₄). Compound 3 was used without further purification. Yield: 4.5 g.

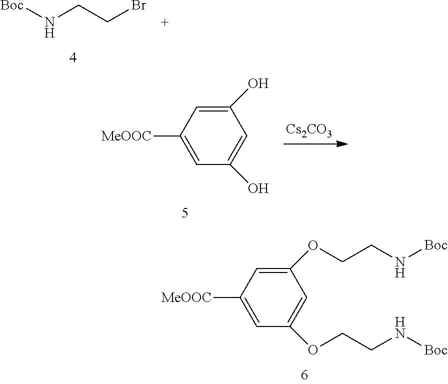

To a solution of compound 5 (29.7354 mmol, 5.0000 g) in 50 mL DMF was added compound 4 (65.4178 mmol, 13.7502 g) and Cs₂CO₃ (118.9414 mmol, 38.7535 g) at room temperature. The reaction mixture was stirred at 60° C. overnight. The reaction mixture was purified by standard work up (1N NaOH, Brine wash, and dried over Na₂SO₄). Compound 6 was purified by silica gel chromatography and concentrated to yield 6.0 g.

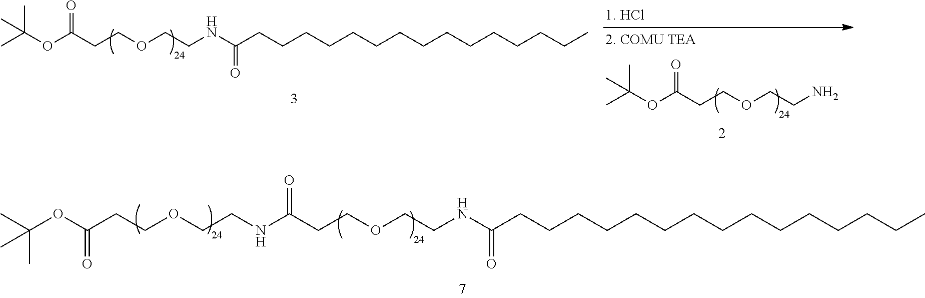

Compound 3 (1.0500 mmol, 1.5129 g) was dissolved in 8 mL 4N HCl/dioxane, and stirred at room temperature for 5 hours. After HCl was removed, compound 2 (1.0000 mmol, 1.2020 g), COMU (1.2000 mmol, 0.5139 g), and TEA (3.0000 mmol, 0.3035 g, 0.418 mL) in DCM was added. The reaction mixture was stirred until full conversion of compound 2 was observed by TLC. The residue was purified by standard work up (1N HCl, sat. NaHCO₃, Brine wash, and dried over Na₂SO₄). Compound 7 was purified by silica gel chromatography and concentrated to yield 2.28 g.

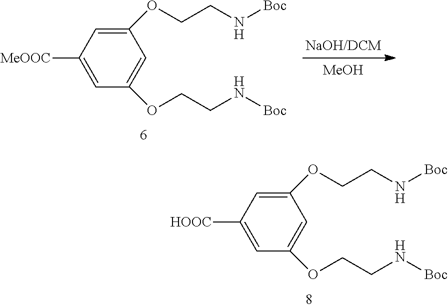 [SMILES: "MeOH" ; COC(=O)c1cc(OCCNC(=O)OC(C)(C)C)cc(OCCNC(=O)OC(C)(C)C)c1 ; CC(C)(C)OC(=O)NCCOc1cc(OCCNC(=O)OC(C)(C)C)cc(C(=O)O)c1]

To a solution of NaOH in 5 mL MeOH was added compound 6 (1.0000 mmol, 0.4545 g) in 20 mL DCM at room temperature. The reaction mixture was stirred at room temperature overnight. The reaction mixture was acidified to pH of 3. The product was dried with Na₂SO₄ to yield 0.200 g a of compound 8 that was used without further purification.

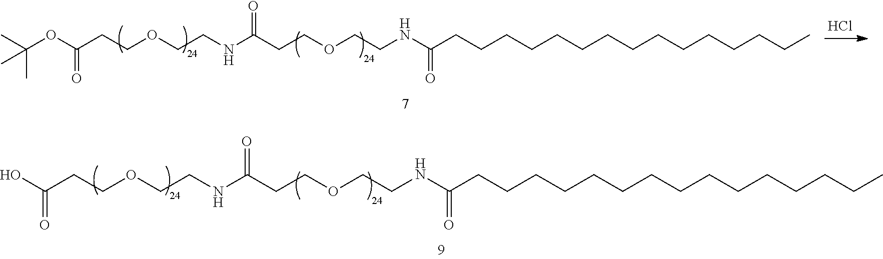

Compound 7 (0.7707 mmol, 1.9800 g) was dissolved in 10 mL 4N HCl/dioxane at room temperature overnight. The solvent was removed and the product was placed under vacuum for 2 hours to yield 1.50 g of compound 9 that was used without further purification.

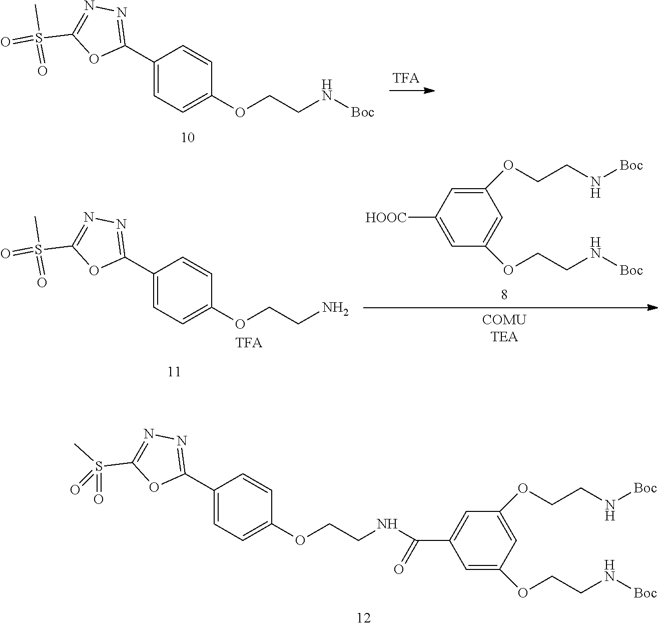 [SMILES: CC(C)(C)OC(=O)NCCOc1ccc(-c2nnc(S(C)(=O)=O)o2)cc1 ; CS(=O)(=O)c1nnc(-c2ccc(OCCN)cc2)o1 ; CC(C)(C)OC(=O)NCCOc1cc(OCCNC(=O)OC(C)(C)C)cc(C(=O)O)c1 ; CC(C)(C)OC(=O)NCCOc1cc(OCCNC(=O)OC(C)(C)C)cc(C(=O)NCCOc2ccc(-c3nnc(S(C)(=O)=O)o3)cc2)c1]

Compound 10 (0.0782 mmol, 0.0300 g) was dissolved in 1 mL DCM, and 0.5 mL TFA was added and the mixture was stirred for 2 hours. TFA was removed and compound 11 was dried under vacuum for 1 hour. Compound 8 (0.0822 mmol, 0.0362 g), COMU (0.0939 mmol, 0.0402 g), and TEA (0.2347 mmol, 0.0237 g, 0.033 mL) were dissolved in 5 mL DCM for 5 min then compound 11 in DCM was added. The reaction mixture was stirred until full conversion of compound 11 was observed by TLC. Compound 12 was purified by silica gel chromatography to yield 0.0135 g.

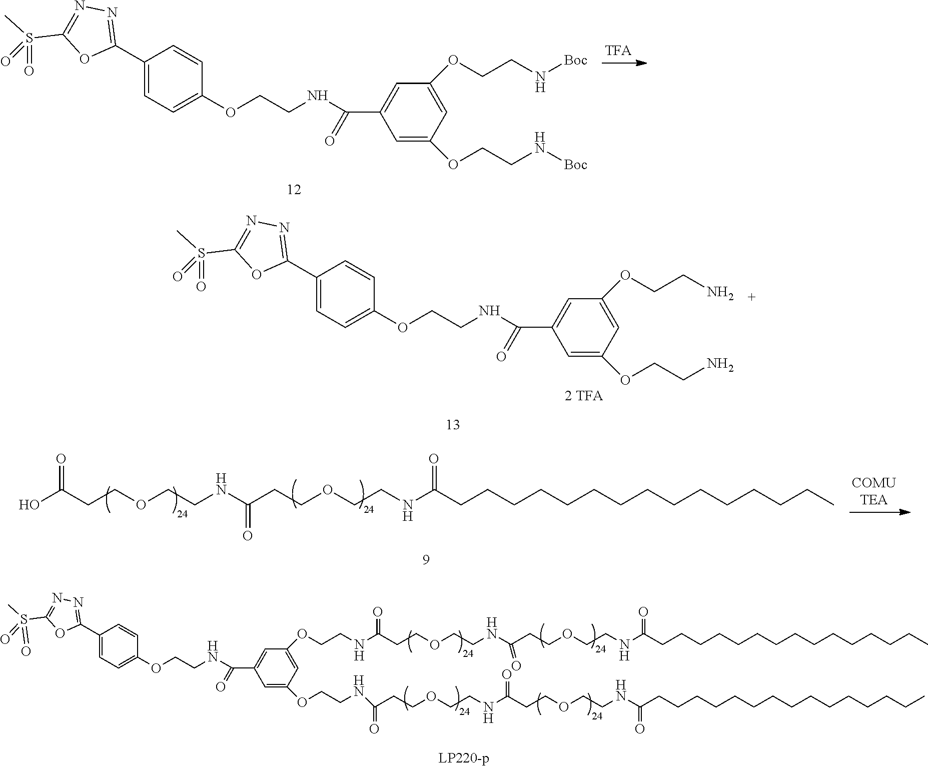

Compound 12 (0.0191 mmol, 0.0135 g) was dissolved in 1 mL DCM, 0.5 mL of TFA was added and the mixture was stirred for 1 hour. TFA was removed and compound 13 was dried under vacuum for 1 hour. Compound 9 (0.0398 mmol, 0.1000 g), COMU (0.0477 mmol, 0.0204 g), and TEA (0.1194 mmol, 0.0121 g, 0.017 mL) was dissolved in 3 mL DCM for 5 minutes, then compound 13 in DCM was added. The mixture was stirred until full conversion of compound 13 was observed by TLC. LP220-p was purified by silica gel chromatography to yield 0.0400 g.

### Synthesis of LP221-p

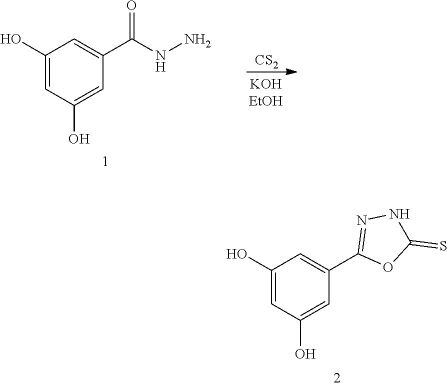

Carbon disulfide (75.0045 mmol, 5.7101 g, 4.532 mL) was slowly added to a solution of compound 1 (25 mmol, 4.20 g) and potassium hydroxide in EtOH (150 mL). The reaction mixture was refluxed for 24 hours. Upon completion, the solvent was evaporated under reduced pressure and the residue was dissolved in water. The aqueous solution was acidified to pH 2 using HCl. The product was extracted with EtOAc, and purified by silica gel chromatography using EtOAc/hexanes. After purification, 3.5 g of Compound 2 was obtained as an orange solid.

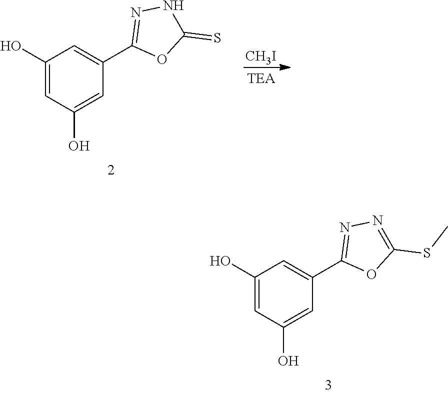

Compound 2 (10.0000 mmol, 2.1021 g) in THF (40 mL) was cooled to 0° C. CH₃I (11.0000 mmol, 1.5609 g, 0.685 mL) was added followed by TEA (10.1000 mmol, 1.0221 g, 1.408 mL). The reaction mixture was stirred for 4 hours. Upon completion, the solvent was quenched by NH₄Cl. The organic phase washed with brine, dried, and purified by silica gel chromatography to yield 1.5 g of compound 3.

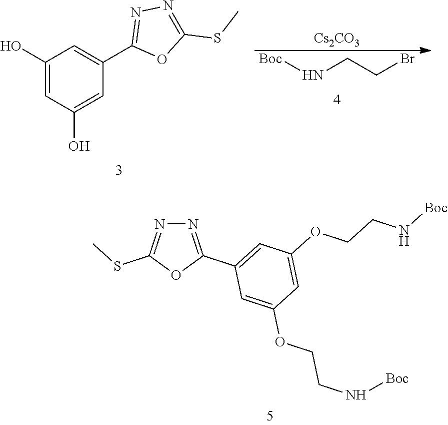

To a solution of compound 4 (6.8679 mmol, 1.5391 g) in 10 mL DMF was added compound 3 (3.1218 mmol, 0.7000 g) and Cs₂CO₃ (9.3654 mmol, 3.0514 g) at room temperature. The reaction mixture was stirred at 60° C. overnight. The reaction mixture was purified by standard work up (1N NaOH, Brine wash, and dried over Na₂SO₄) and silica gel chromatography to yield 1.0 g of compound 5.

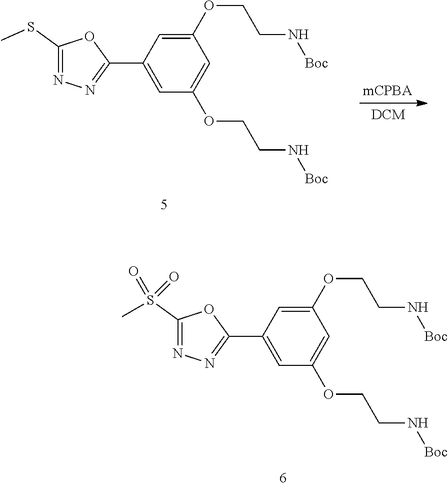 [SMILES: CSc1nnc(-c2cc(OCCNC(=O)OC(C)(C)C)cc(OCCNC(=O)OC(C)(C)C)c2)o1 ; CC(C)(C)OC(=O)NCCOc1cc(OCCNC(=O)OC(C)(C)C)cc(-c2nnc(S(C)(=O)=O)o2)c1]

A mixture of compound 5 (0.2000 mmol, 0.1021 g) and mCPBA (0.9998 mmol, 0.1725 g) in DCM was stirred until full conversion of mCPBA was observed by TLC. The reaction mixture was purified by standard work up (1N HCl, sat. NaHCO₃, Brine wash, and dried over Na₂SO₄) and silica gel chromatography to yield 0.05 g of compound 6.

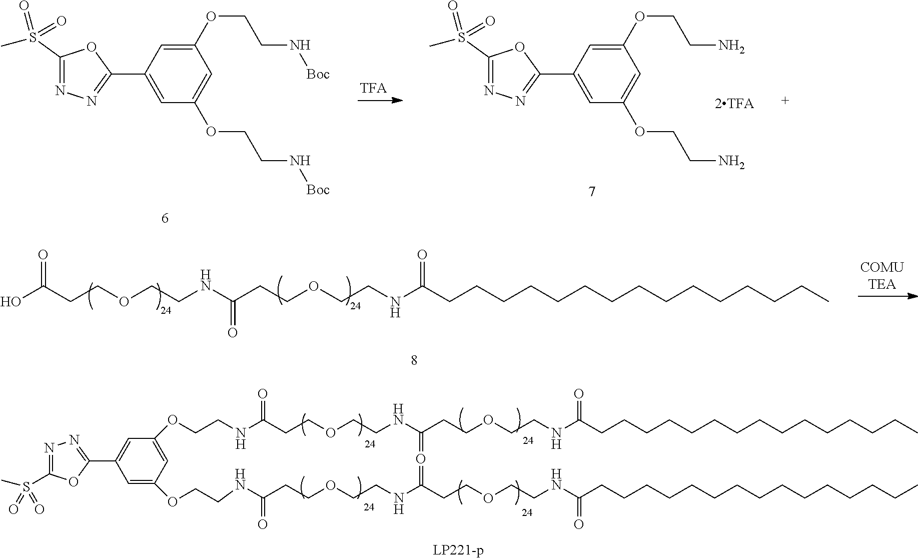

Compound 6 (0.0191 mmol, 0.0104 g) was dissolved in 1 mL DCM, and 0.5 mL of TFA was added and the mixture was stirred for 1 hour. All of the TFA was removed, and compound 7 was dried under vacuum for 1 hour. Compound 8 (0.0398 mmol, 0.1000 g), COMU (0.0477 mmol, 0.0204 g), and TEA (0.1990 mmol, 0.0201 g, 0.028 mL) was dissolved in 3 mL DCM for 5 minutes then compound 7 in DCM was added. The reaction mixture was stirred until full conversion of compound 7 was observed by TLC. The residue was purified by silica gel chromatography to yield 0.016 g of LP221-p.

### Synthesis of LP223-p

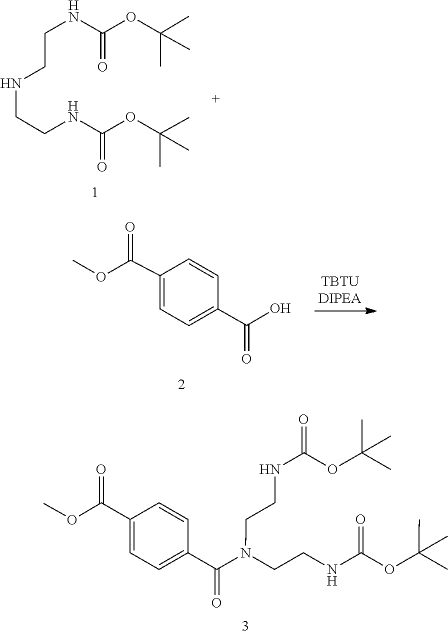 [SMILES: CC(C)(C)OC(=O)NCCNCCNC(=O)OC(C)(C)C ; COC(=O)c1ccc(C(=O)O)cc1 ; COC(=O)c1ccc(C(=O)N(CCNC(=O)OC(C)(C)C)CCNC(=O)OC(C)(C)C)cc1]

To a solution of compound 1 (741 mg, 2.442 mmol, 1.0 equiv.), compound 2 (528 mg, 2.930 mmol, 1.20 equiv.) and diisopropylethylamine (1.276 mL, 7.327 mmol, 3.0 equiv.) in anhydrous DMF (10 mL) was added TBTU (980 mg, 3.052 mmol, 1.25 equiv.) at room temperature. The reaction mixture was kept at room temperature for 2 hours. The organic phase was quenched with saturated sodium bicarbonate aqueous solution (10 mL) and extracted with EtOAc (2×10 mL). The organic phases were combined, dried over anhydrous Na₂SO₄, and concentrated. Compound 3 was purified by CombiFlash® and was eluted with 40-80% EtOAc in hexanes. LC-MS: [M+H]+, calculated 466.25, found 466.72.

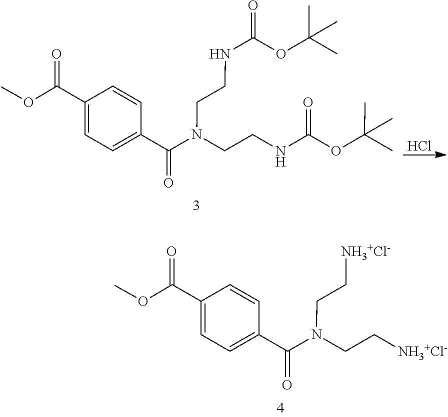

To compound 3 (990 mg, 2.126 mmol, 1.0 equiv.) was added 4M HCl in dioxane (6.38 mL, 25.518 mmol, 12 equiv.) at room temperature. The reaction mixture was kept at room temperature for 1 hour and then concentrated. Compound 4 was used directly without further purification. LC-MS: [M+H]/+ calculated 266.14, found 266.43.

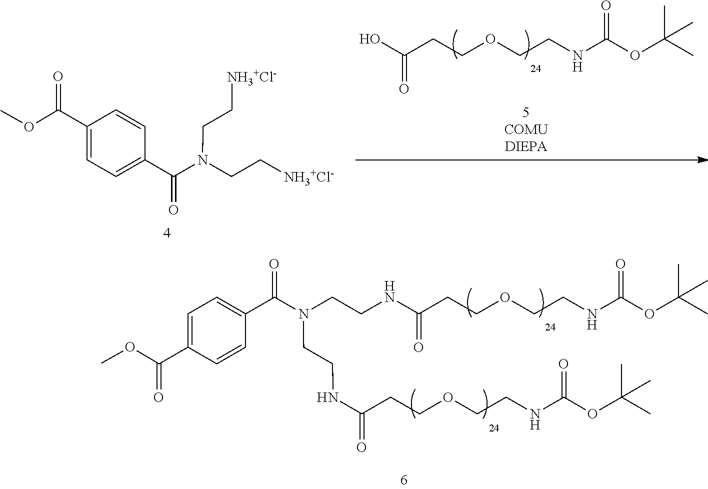

To a solution of compound 4 (100 mg, 0.295 mmol, 1.0 equiv.), compound 5 (755 mg, 0.606 mmol, 2.05 equiv.) and diisopropylethylamine (0.257 mL, 0.025 mmol, 5.0 equiv.) in anhydrous DCM (10 mL) was added COMU (278 mg, 0.650 mmol, 2.20 equiv.) at room temperature. The reaction mixture was kept at room temperature for 1 hour. The reaction mixture was washed with saturated ammonium chloride (10 mL) and sodium bicarbonate aqueous solution (10 mL). The organic phase was dried over anhydrous Na₂SO₄ and concentrated. Compound 6 was purified by CombiFlash® eluting with 8-18% MeOH in DCM. LC-MS: [M+3H]/3, calculated 907.86, found 907.61.

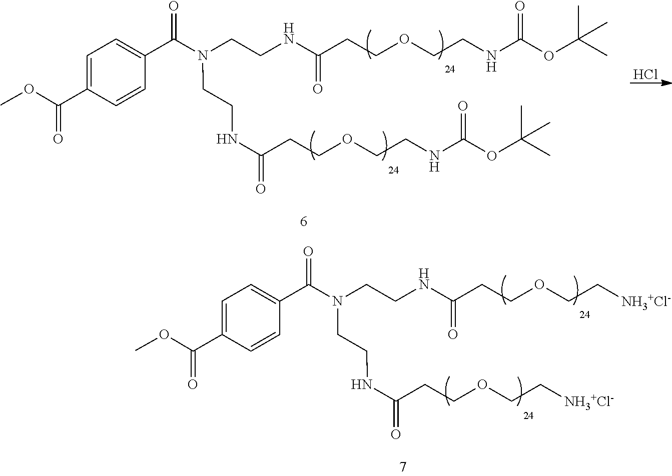

To compound 6 (550 mg, 0.202 mmol, 1.0 equiv.) was added 4M HCl in dioxane (1.01 mL, 4.040 mmol, 20 equiv.) at room temperature. The reaction mixture was kept at room temperature for 1 hour and then concentrated. Compound 7 was used directly without further purification. LC-MS: [M+H]/+ calculated 841.16, found 842.20.

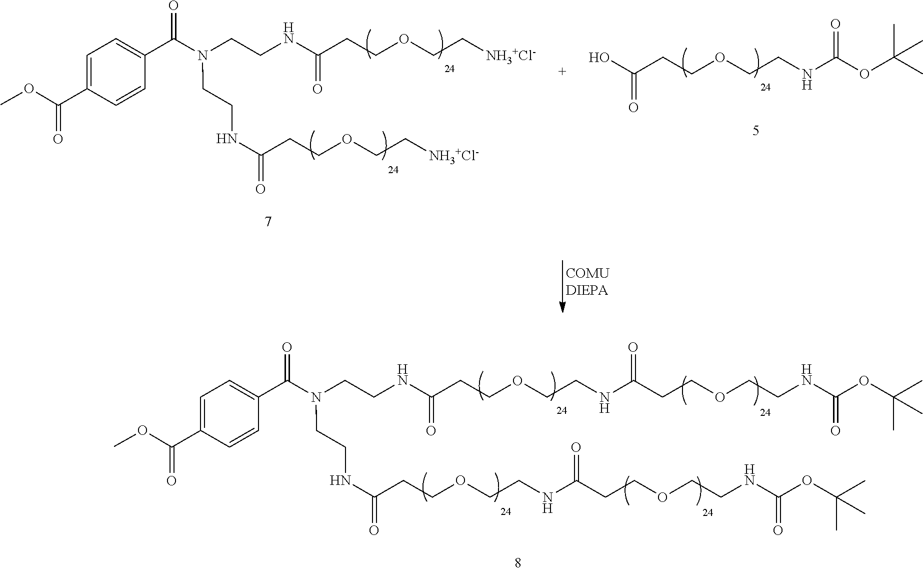

To a solution of compound 1 (490 mg, 0.188 mmol, 1.0 equiv.), compound 5 (482 mg, 0.387 mmol, 2.05 equiv.) and diisopropylethylamine (0.164 mL, 0.944 mmol, 5.0 equiv.) in anhydrous DCM (10 mL) was added COMU (177 mg, 0.415 mmol, 2.20 equiv.) at room temperature. The reaction mixture was kept at room temperature for 1 hour. The reaction mixture was washed with saturated ammonium chloride (10 mL) and sodium bicarbonate aqueous solution (10 mL). The organic phase was dried over anhydrous Na₂SO₄ and concentrated. Compound 8 was purified by CombiFlash® eluting with 8-20% MeOH in DCM. LC-MS: [M+5H]/5, calculated 960.18 found 961.74.

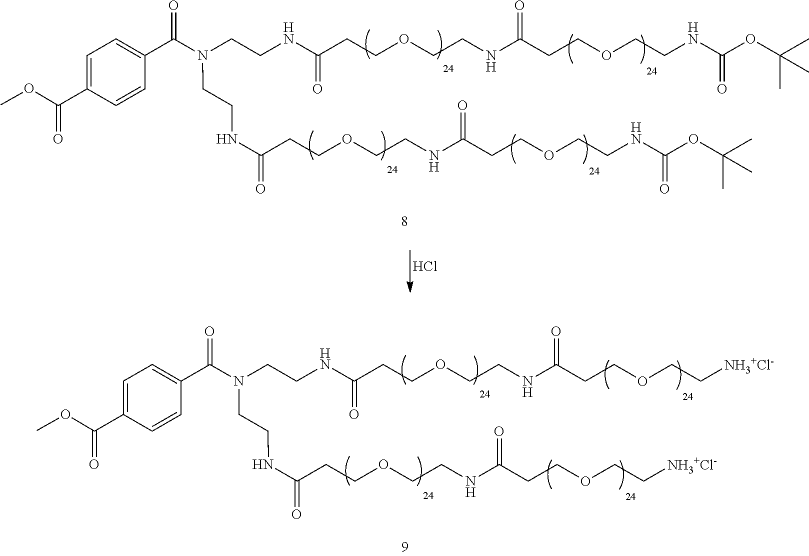

To compound 1 (670 mg, 0.134 mmol, 1.0 equiv.) was added 4M HCl in dioxane (0.673 mL, 2.691 mmol, 20 equiv.) at room temperature. The reaction mixture was kept at room temperature for 1 hour and then concentrated. Compound 9 was used directly without further purification. LC-MS: [M+5H]/5 calculated 956.16, found 957.66.

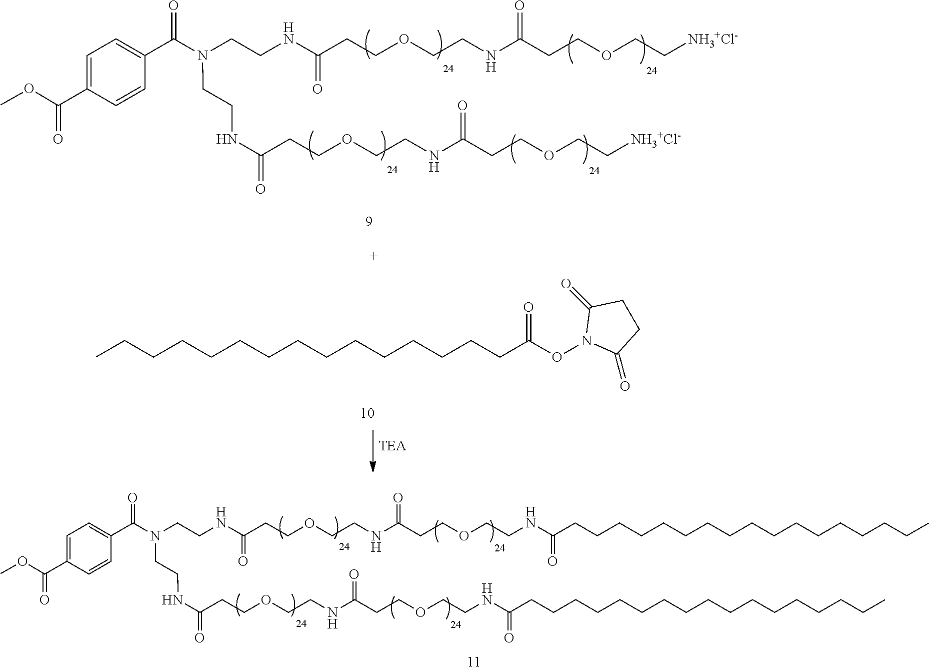

To a solution of compound 9 (650 mg, 0.134 mmol, 1.0 equiv.) and compound 10 (106 mg, 0.301 mmol, 2.25 equiv.) in anhydrous DCM (20 mL) was added TEA (0.095 mL, 0.669 mmol, 5.0 equiv.) at room temperature. The reaction mixture was kept at room temperature for 2 hours and the solvent was concentrated. Compound 11 was separated by CombiFlash® eluting with 8-20% MeOH in DCM. LC-MS: calculated [M+5H]/5 1051.45, found 1053.44.

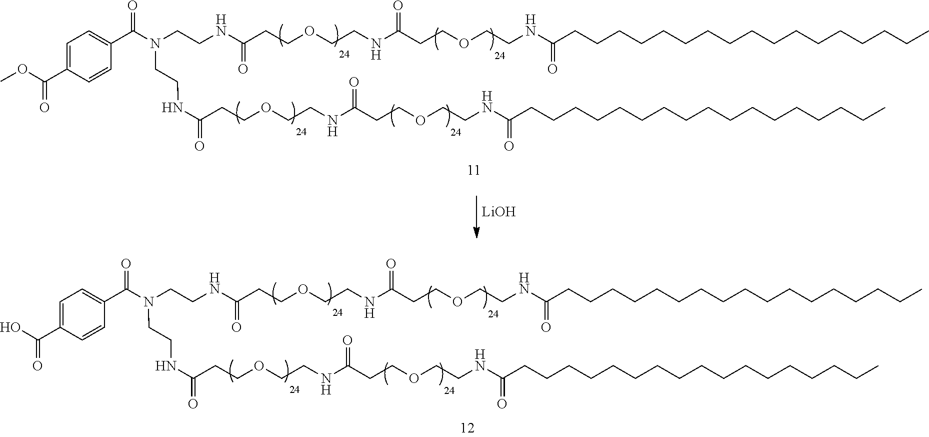

To a solution of compound 11 (460 mg, 0.0875 mmol, 1.0 equiv.) in THF (5 mL) and water (5 mL) was added LiOH (10.5 mg, 0.437 mmol, 5.0 equiv.) at room temperature. The reaction mixture was kept at room temperature for 1 hour. The reaction mixture pH was adjusted to 3.0 by adding HCl and was extracted with DCM (2×10 mL). The combined organic phases were dried over anhydrous Na₂SO₄ and concentrated. Compound 12 was used directly without further purification. LC-MS: calculated [M+5H]+/5 1048.65, found 1050.68.

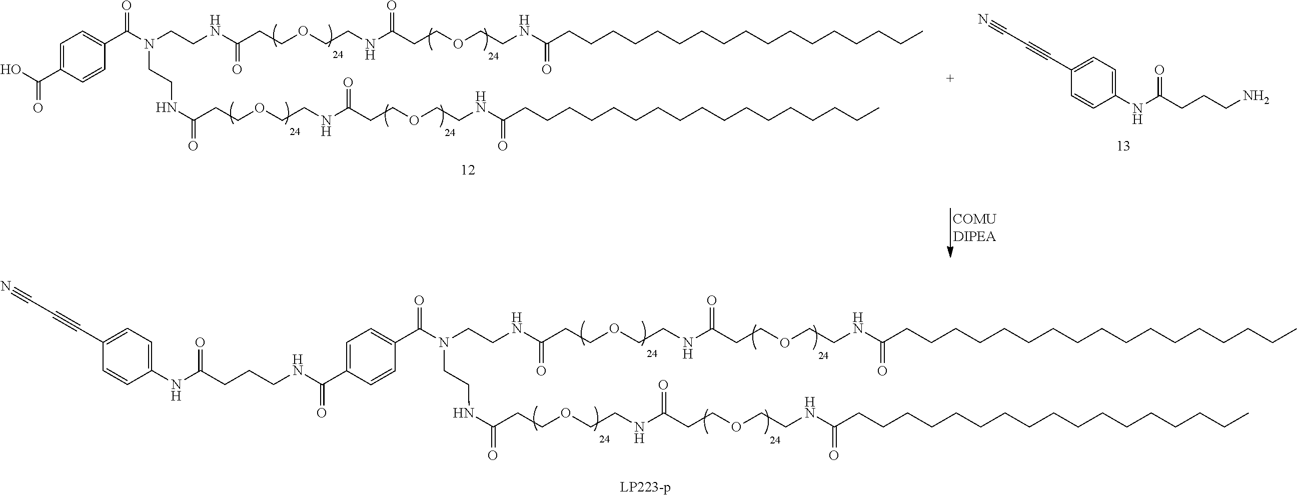

To a solution of compound 12 (100 mg, 0.0191 mmol, 1.0 equiv.), compound 13 (4.8 mg, 0.021 mmol, 1.1 equiv.) and diisopropylethylamine (0.010 mL, 0.0572 mmol, 3.0 equiv.) in anhydrous DCM (3 mL) was added COMU (10.2 mg, 0.0238 mmol, 1.25 equiv.) at room temperature. The reaction mixture was kept at room temperature for 1 hour. The reaction mixture was washed with saturated sodium bicarbonate aqueous solution (5 mL). The organic phase was dried over anhydrous Na₂SO₄ and concentrated. LP223-p was purified by CombiFlash® eluting with 8-20% MeOH in DCM. LC-MS: [M+5H]/5, calculated 1090.47, found 1091.85.

### Synthesis of LP224-p

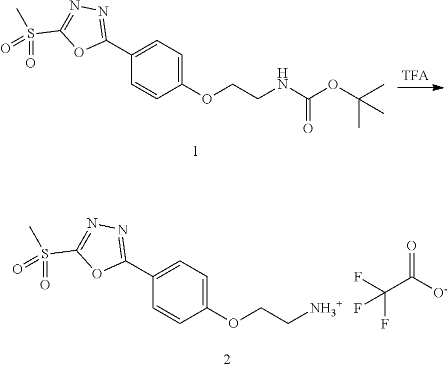 [SMILES: CC(C)(C)OC(=O)NCCOc1ccc(-c2nnc(S(C)(=O)=O)o2)cc1 ; CS(=O)(=O)c1nnc(-c2ccc(OCC[NH3+])cc2)o1 ; O=C([O-])C(F)(F)F]

To solution of compound 1 (12 mg, 0.0313 mmol, 1.0 equiv.) in DCM (1 mL) was added TFA (0.5 mL) at room temperature. The reaction mixture was kept at room temperature for 30 minutes and then concentrated. Compound 2 was used directly without further purification. LC-MS: [M+H]+ calculated 284.06, found 284.26.

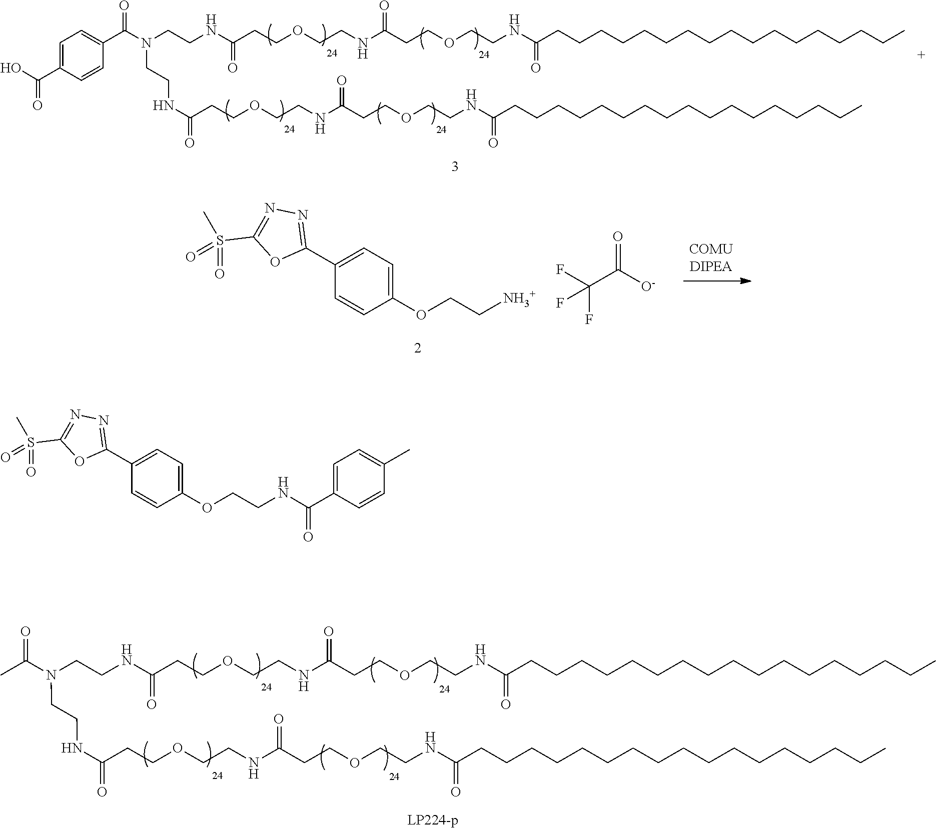

To a solution of compound 3 (150 mg, 0.0286 mmol, 1.0 equiv., compound 12 from LP223-p synthesis), compound 2 (12.5 mg, 0.0315 mmol, 1.1 equiv.) and diisopropylethylamine (0.015 mL, 0.0859 mmol, 3.0 equiv.) in anhydrous DCM (3 mL) was added COMU (15.3 mg, 0.0358 mmol, 1.25 equiv.) at room temperature. The reaction mixture was kept at room temperature for 1 hour. The reaction mixture was washed with saturated sodium bicarbonate aqueous solution (5 mL). The organic phase was dried over anhydrous Na₂SO₄ and concentrated. LP224-p was purified by CombiFlash® eluting with 8-16% MeOH in DCM. LC-MS: [M+5H]/5, calculated 1101.66, found 1103.13.

### Synthesis of LP225-p

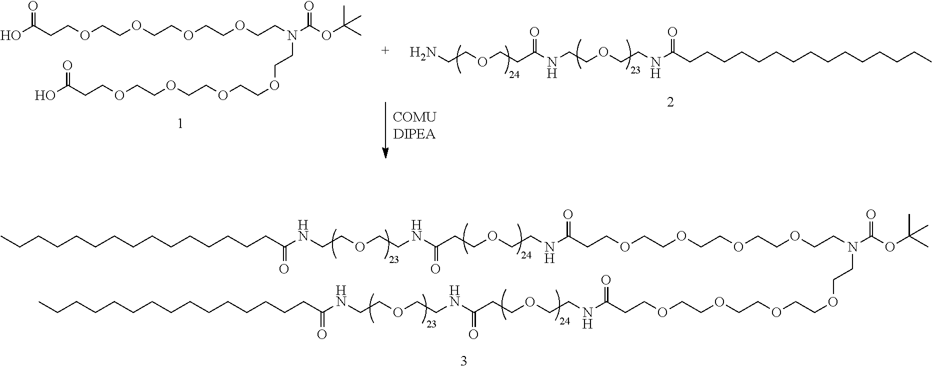

To a solution of compound 1 (80 mg, 0.130 mmol, 1.0 equiv.), compound 2 (652 mg, 0.267 mmol, 2.05 equiv.), and diisopropylethylamine (0.068 mL, 0.391 mmol, 3.0 equiv.) in anhydrous DCM (10 mL) was added COMU (134 mg, 0.312 mmol, 2.40 equiv.) at room temperature. The reaction mixture was kept at room temperature overnight. Compound 3 was purified by CombiFlash® eluting with 8-16% MeOH in DCM. LC-MS: [M+5H]/5, calculated 1091.89, found 1093.41.

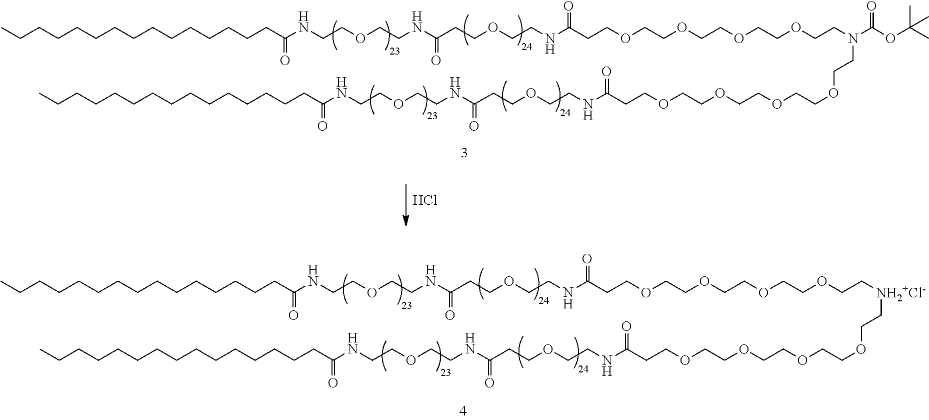

To compound 3 (340 mg, 0.0623 mmol, 1.0 equiv.) was added 4M HCl in dioxane (0.311 mL, 1.245 mmol, 20 equiv.) at room temperature. The reaction mixture was kept at room temperature for 1 hour and then concentrated. Compound 4 was used directly without further purification. LC-MS: [M+5H]/5 calculated 1071.88, found 1073.36.

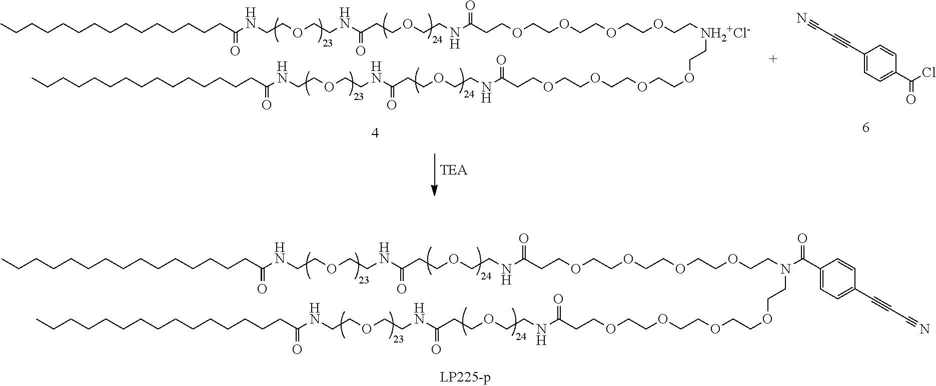

To a solution of compound 4 (100 mg, 0.0185 mmol, 1.0 equiv.) and compound 5 (3.9 mg, 0.0204 mmol, 1.10 equiv.) in anhydrous DCM (2 mL) was added TEA (0.008 mL, 0.0556 mmol, 3.0 equiv.) at room temperature. The reaction mixture was kept at room temperature for 2 hours and the solvent was concentrated. LP225-p was separated by CombiFlash® eluting with 13-20% MeOH in DCM. LC-MS: calculated [M+5H]/5 1102.48, found 1104.45.

### Synthesis of LP226-p

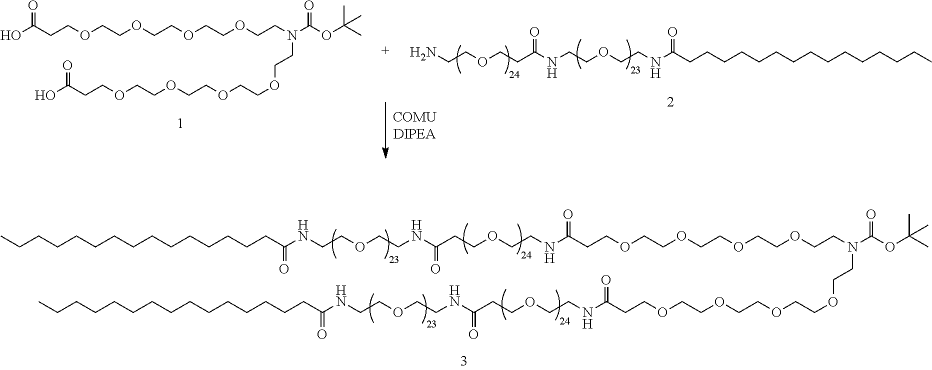

To a solution of compound 1 (80 mg, 0.130 mmol, 1.0 equiv.), compound 2 (652 mg 0.267 mmol, 2.05 equiv.) and diisopropylethylamine (0.068 mL, 0.391 mmol, 3.0 equiv.) in anhydrous DCM (10 mL) was added COMU (134 mg, 0.312 mmol, 2.40 equiv.) at room temperature. The reaction mixture was kept at room temperature overnight. Compound 3 was purified by CombiFlash® eluting with 8-16% MeOH in DCM. LC-MS: [M+5H]/5, calculated 1091.89, found 1093.41.

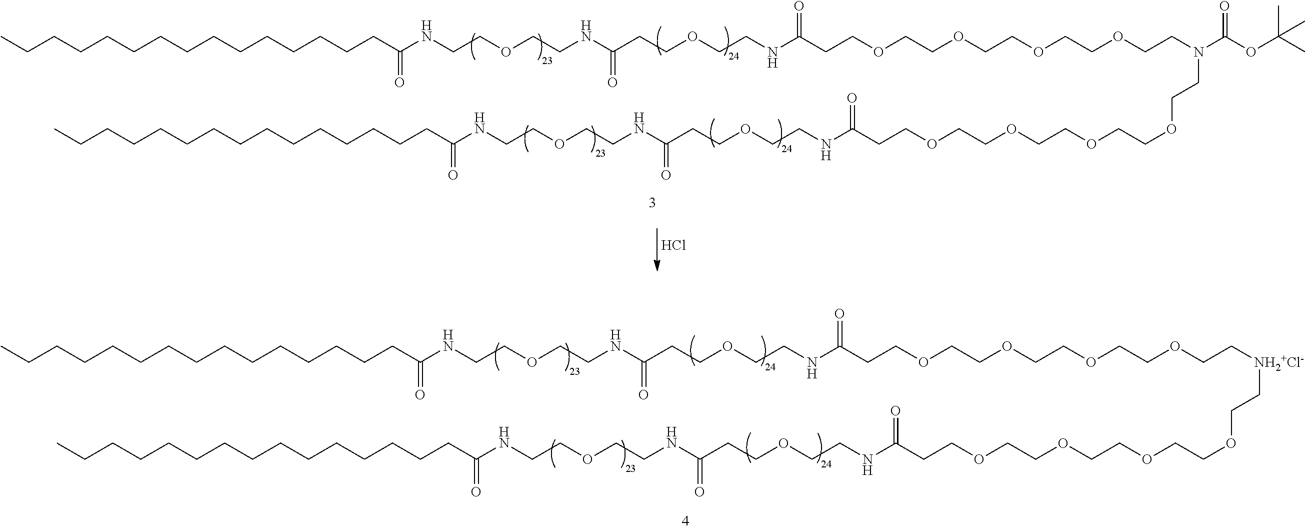

To compound 3 (340 mg, 0.0623 mmol, 1.0 equiv.) was added 4M HCl in dioxane (0.311 mL, 1.245 mmol, 20 equiv.) at room temperature. The reaction mixture was kept at room temperature for 1 hour and then concentrated. Compound 4 was used directly without further purification. LC-MS: [M+5H]/5 calculated 1071.88, found 1073.36.

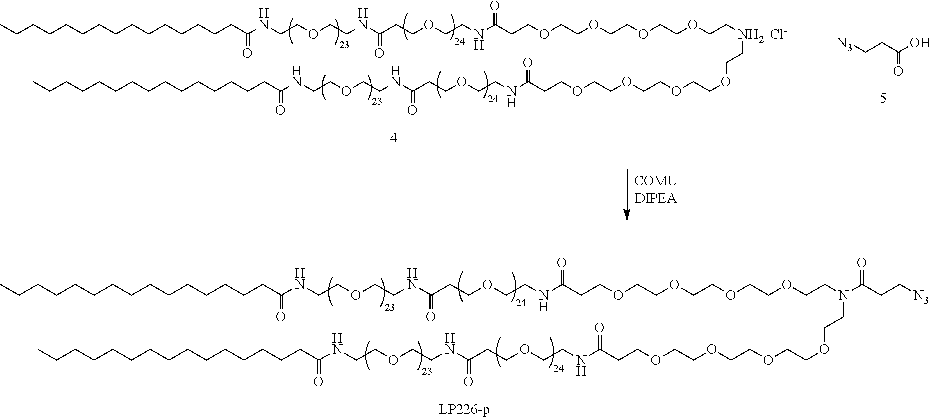

To a solution of compound 4 (80 mg, 0.0148 mmol, 1.0 equiv.), compound 5 (1.9 mg, 0.0163 mmol, 1.1 equiv.), and diisopropylethylamine (0.008 mL, 0.0445 mmol, 3.0 equiv.) in anhydrous DCM (2 mL) was added COMU (7.9 mg, 0.0185 mmol, 1.25 equiv.) at room temperature. The reaction mixture was kept at room temperature for 1 hour. The reaction mixture was washed with saturated sodium bicarbonate aqueous solution (5 mL). The organic phase was dried over anhydrous Na₂SO₄ and concentrated. LP226-p was purified by CombiFlash® eluting with 15-20% MeOH in DCM. LC-MS: [M+5H]/5, calculated 1091.28, found 1093.41.

### Synthesis of LP238-p

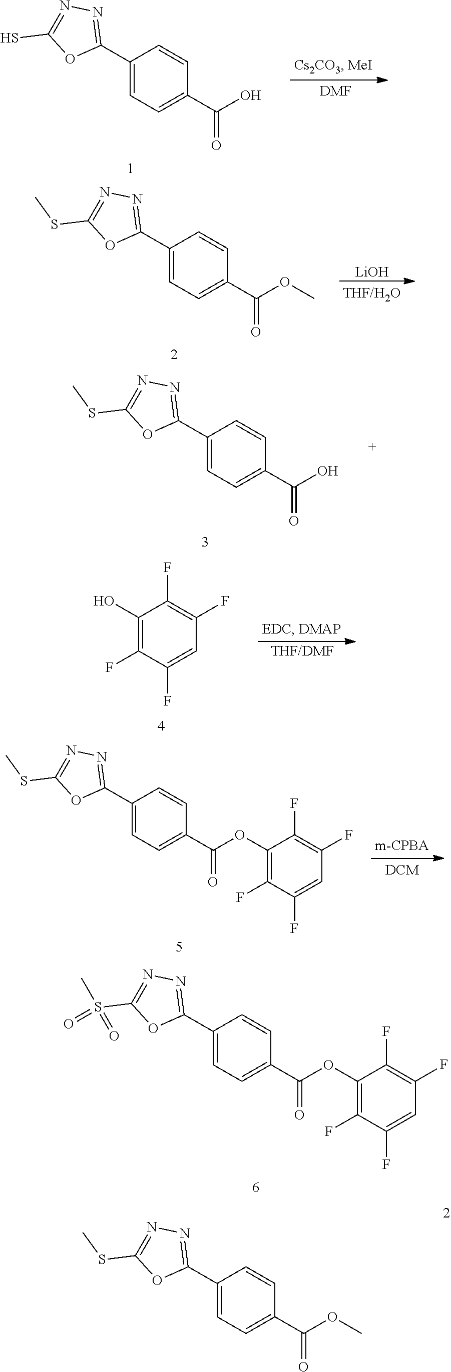

To a suspension of compound 1 (5.00 g, 22.50 mmol) and Cs₂CO₃ (25.66 g, 78.75 mmol) in anhydrous DMF (80 mL) was added methyl iodide (4.20 mL, 67.50 mmol) at room temperature. The reaction mixture was stirred at room temperature for 48 hours. The reaction mixture was quenched with water (200 mL) and the mixture was extracted with EtOAc (3×100 mL). The organic phase was combined and washed with water and brine. The organic layer was dried over anhydrous Na₂SO₄ and concentrated. Compound 2 was obtained as a light yellow solid, 5.41 g, 96%. Compound 2 was used directly without further purification. LC-MS: [M+H] calculated 251.05, found 251.18.

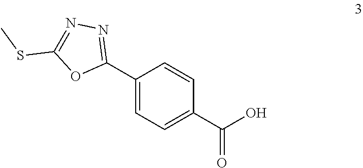 [SMILES: CSc1nnc(-c2ccc(C(=O)O)cc2)o1]

To a solution of compound 2 (5.41 g, 21.62 mmol) in THF/H₂O (50 mL/50 mL) was added LiOH (2.59 g, 108.08 mmol) at room temperature. The reaction mixture was stirred at room temperature for 1 hour. After removing THF under vacuum, the pH was adjusted to approximately 2 by [C] HCl. Then EtOAc (3×60 mL) was used to extract. The organic layers were combined, washed with brine, then dried over anhydrous Na₂SO₄, and concentrated. Compound 3 was obtained as an off-white solid, 5 g, 98%. Compound 3 was used directly without further purification. LC-MS: calculated [M+H] 237.03, found 237.26.

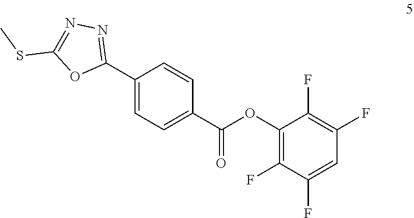 [SMILES: CSc1nnc(-c2ccc(C(=O)Oc3c(F)c(F)cc(F)c3F)cc2)o1]

To a solution of compound 3 (5.81 g, 24.60 mmol) in THF/DMF (80 mL/20 mL) was added EDC (7.07 g, 36.90 mmol), DMAP (0.30 g, 2.46 mmol) and compound 4 (6.13 g, 36.90 mmol) at room temperature. The reaction mixture was stirred at room temperature overnight. After removing solvent under vacuum, the residue was loaded on a 120 g column and compound 5 was eluted with 0-50% EtOAc in hexanes. Compound 5 was obtained as a white solid, 9.36 g, 99%. LC-MS: calculated [M+H] 385.03, found 385.46.

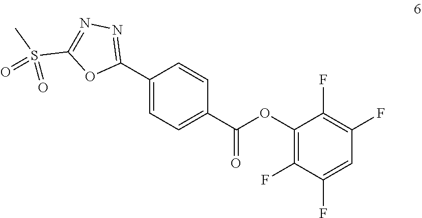 [SMILES: CS(=O)(=O)c1nnc(-c2ccc(C(=O)Oc3c(F)c(F)cc(F)c3F)cc2)o1]

To a solution of compound 5 (2.29 g, 5.96 mmol) in DCM (110 mL) was added 70% m-CPBA (5.14 g, 27.79 mmol) at 0° C. The reaction mixture was stirred at room temperature for 6 hours. Another 1.8 g m-CPBA was added at room temperature. The reaction mixture was stirred at room temperature overnight. After filtration, the solvent was removed under vacuum. The residue was recrystallized from DCM/EtOAc (50 mL/50 mL) twice. Compound 6 was obtained as white needle crystals, 1.93 g, 78%. LC-MS: calculated [M+H] 417, found 417.

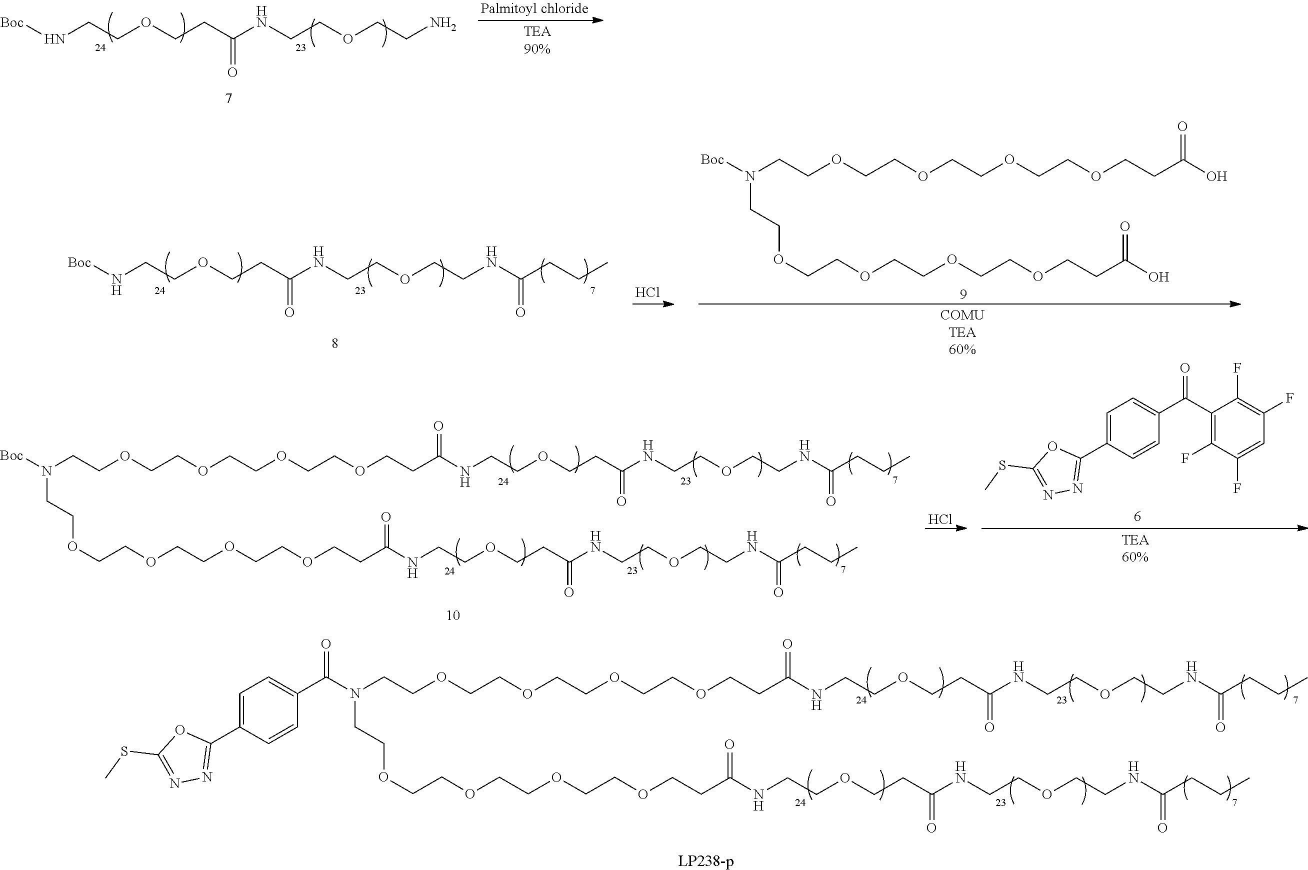

To a solution of compound 7 (10.00 g, 4.34 mmol) in DCM (100 mL) was added palmitoyl chloride (1.31 g, 4.78 mmol) and TEA at 0° C. The reaction mixture was stirred at room temperature overnight and then the solvent was removed under vacuum. The residue was purified by silica gel chromatography using 0-20% MeOH in DCM. Compound 8 was obtained as a white solid, 10.0 g, 90%.

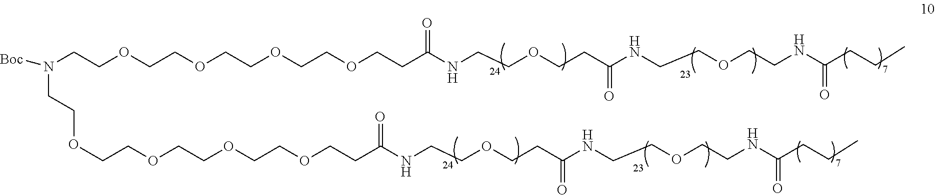

Compound 8 (9.56 g, 3.76 mmol) was dissolved in 25 mL 4N HCl/dioxane and stirred at room temperature for 1 hour. All solvent was removed and the residue was dried under vacuum for 2 hours. The residue was re-dissolved in 150 mL DCM and TEA was added, followed by compound 9 (1.10 g, 1.79 mmol), and COMU (1.69 g, 3.94 mmol). The reaction mixture was stirred at room temperature overnight. After a standard workup (1N HCl, Sat. bicarb, brine wash), DCM was removed. Compound 10 was purified by a 120 g column using 0-20% MeOH in DCM to obtain 5.90 g, 60%.

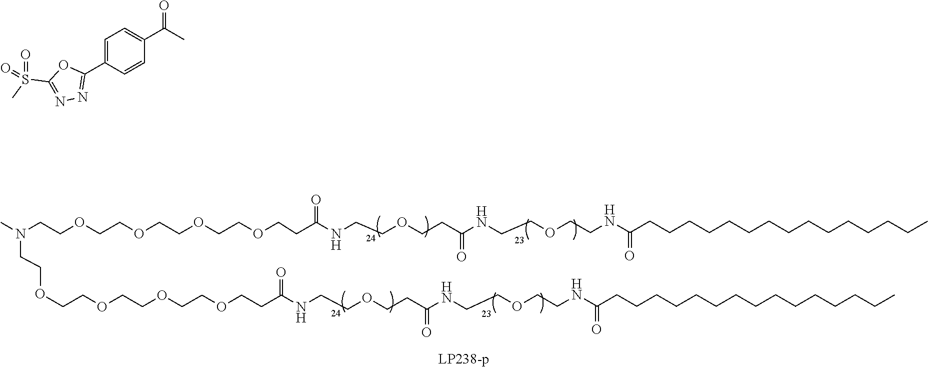

Compound 10 (4.50 g, 0.82 mmol) was dissolved in 20 mL 4N HCl/dioxane and stirred at room temperature for 1 hour. All solvent was removed and the residue was dried under vacuum for 2 hours. The residue was re-dissolved in 100 mL DCM and TEA was added, followed by compound 6 (0.69 g, 1.65 mmol). The reaction mixture was stirred at room temperature overnight. TEA was removed by a 1H HCl wash and the organic layer was concentrated. Crude LP238-p was purified by silica gel chromatography using 0-20% MeOH in DCM. 2.80 g (60%) of LP238-p was obtained as a light yellow solid.

### Synthesis of LP240-p

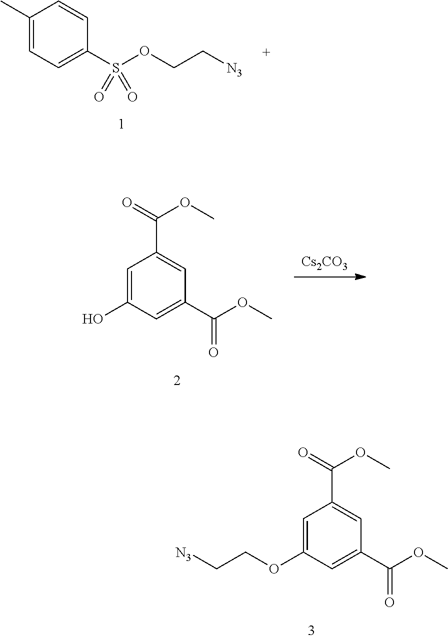 [SMILES: Cc1ccc(S(=O)(=O)OCCN=[N+]=[N-])cc1 ; COC(=O)c1cc(O)cc(C(=O)OC)c1 ; "Cs2CO3" ; COC(=O)c1cc(OCCN=[N+]=[N-])cc(C(=O)OC)c1]

To a suspension of compound 1 (880 mg, 3.647 mmol, 1.0 equiv.) and Cs₂CO₃ (1.782 g, 5.471 mmol, 1.50 equiv.) in anhydrous DMF (10 mL) was added compound 2 (0.843 g, 4.012 mmol, 1.10 equiv.) at room temperature. The reaction mixture was kept at room temperature for 3 hours. The reaction mixture was quenched with water (20 mL) and the mixture was extracted with ethyl acetate (2×10 mL). The combined organic phases were washed with brine (1×20 mL) and water (1×20 mL). The organic phase was dried through anhydrous Na₂SO₄ and concentrated. Compound 3 was used directly without further purification. LC-MS: [M+H]+ calculated 280.09, found 280.39.

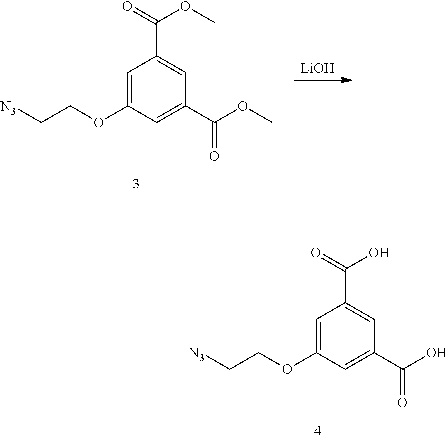 [SMILES: COC(=O)c1cc(OCCN=[N+]=[N-])cc(C(=O)OC)c1 ; "LiOH" ; [N-]=[N+]=NCCOc1cc(C(=O)O)cc(C(=O)O)c1]

To a solution of compound 3 (1000 mg, 3.581 mmol, 1.0 equiv.) in THF (10 mL) and water (10 mL) was added LiOH (686 mg, 19.978 mmol, 8.0 equiv.) at room temperature. The reaction mixture was kept at room temperature for 1 hour. The reaction mixture pH was adjusted to 1.0 by adding HCl. The product was extracted with ethyl acetate (2×10 mL). The combined organic phases were dried over anhydrous Na₂SO₄ and concentrated. Compound 4 was used directly without further purification. LC-MS: calculated [M+H]+ 252.05, found 251.31.

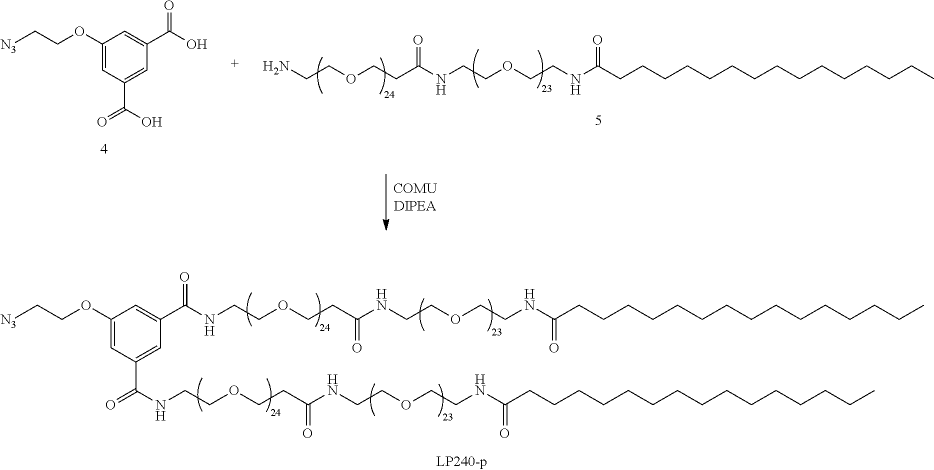

To a solution of compound 4 (5 mg, 0.0199 mmol, 1.0 equiv.), compound 5 (100 mg, 0.0408 mmol, 2.05 equiv.), and diisopropylethylamine (0.017 mL, 0.0995 mmol, 5.0 equiv.) in anhydrous DCM (2 mL) was added COMU (20.5 mg, 0.0478 mmol, 2.40 equiv.) at room temperature. The reaction mixture was kept at room temperature overnight. LP240-p was purified by CombiFlash® eluting with 8-20% MeOH in DCM LC-MS. [M+5H]/5, calculated 1019.43, found 1020.79.

### Synthesis of LP246-p

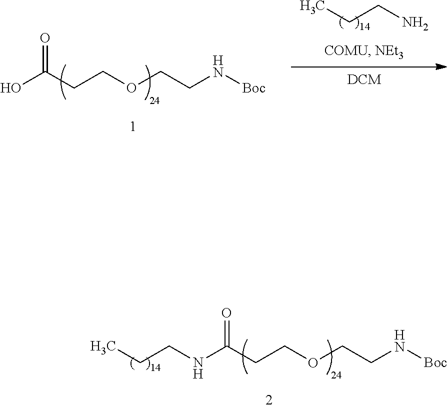

Compound 1 (0.5 g, 0.401 mmol) and COMU (0.206 g, 0.481 mmol) were dissolved in DCM (10 mL) and NEt₃ (0.168 mL, 1.2 mmol) was added. The resulting solution was stirred for 10 minutes. After 10 minutes 1-aminohexadecane (0.102 g, 0.42 mmol) was added to the solution of compound 1 and COMU. The resulting solution was stirred for 90 minutes and then checked by LC-MS. The reaction mixture was quenched with 5 mL of water and stirred for 5 minutes. The layers were separated and the organic layer was washed with 1 M HCl(aq) (2×15 mL), sat. NaHCO₃(aq) (2×20 mL), water (20 mL), sat. NaCl(aq) (2×20 mL), dried over Na₂SO₄ and concentrated to yield a foamy light yellow solid. Crude product was purified by silica gel chromatography 0-20% MeOH in DCM. Pure fractions of compound 2 were combined to yield 515 mg (87% yield) as a white solid.

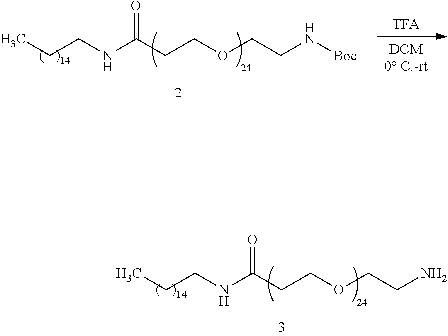

Compound 2 (0.515 g, 0.35 mmol) was dissolved in DCM (4 mL), cooled to 0° C., and TFA (1 mL, 13 mmol) was added. After the addition of the TFA, the reaction mixture was allowed to warm to room temperature. The resulting solution was stirred for 90 minutes and then analyzed by LC-MS. The reaction mixture was quenched with the addition of sat. NaHCO₃(aq) until no effervescence was observed and stirred for 5 minutes. The layers were separated, and the organic layer was washed with sat. NaHCO₃(aq) (2×20 mL), water (20 mL), sat. NaCl(aq) (20 mL), dried over Na₂SO₄ and concentrated to yield compound 3 as a foamy white solid 0.4674 g (97.4% yield).

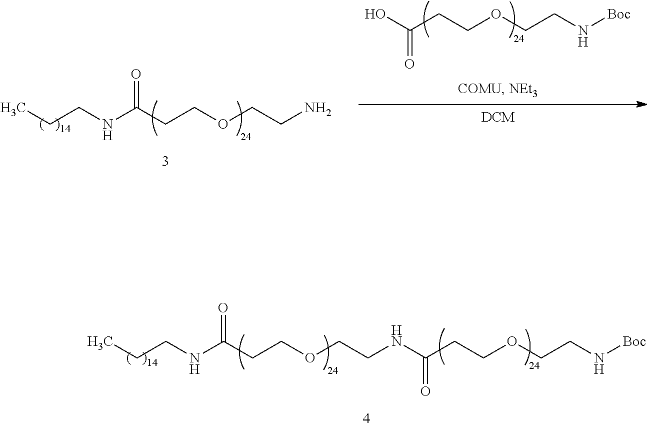

^{t}Boc-amido-PEG24-COOH (0.524 g, 0.42 mmol) and COMU (0.180 g, 0.42 mmol) were dissolved in DCM (10 mL) and NEt₃ (0.488 mL, 3.5 mmol) was added. The resulting solution was stirred for 10 minutes. After 10 minutes, compound 3 (0.480 g, 0.35 mmol) was added to the solution of tBoc-amido-PEG24-COOH. The resulting solution was stirred for 1 hour and checked with LC-MS. The reaction mixture was quenched with 5 mL of water and stirred for 5 minutes. The layers were separated, and the organic layer was washed with 1 M HCl (1×15 mL), sat. NaHCO_{3(aq)} (2×20 mL), Water (20 mL), 1 M HCl (1×20 mL), sat. NaCl(aq) (2×20 mL), dried over Na₂SO₄ and concentrated to yield a foamy light yellow solid (ca. 900 mg). Crude product was purified by silica gel chromatography 0-20% MeOH in DCM. Compound 4 eluted at 4% MeOH in DCM. Pure fractions of compound 4 were combined to yield 0.780 g (85.7%) as a light pink solid.

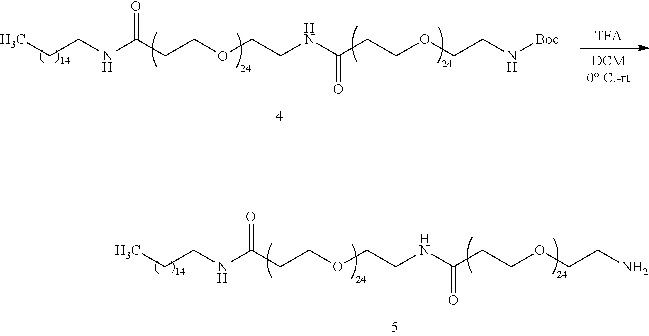

Compound 4 (0.78 g, 0.3 mmol) was dissolved in DCM (4 mL), cooled to 0° C., and TFA (1 mL, 13 mmol) was added. After the addition of the TFA, the reaction mixture was allowed to warm to room temperature. The resulting solution was stirred for 3 hours and checked by LC-MS. The reaction mixture was quenched with the addition sat. NaHCO₃(aq) until no effervescence was observed and stirred for 5 minutes. The layers were separated and the organic layer was washed with sat. NaHCO₃(aq)(2×20 mL), water (20 mL), sat. NaCl(aq) (20 mL), dried over Na₂SO₄ and concentrated to yield compound 5 as a foamy white solid 0.741 g (98.9% yield). Compound 5 was used in the next step without further purification.

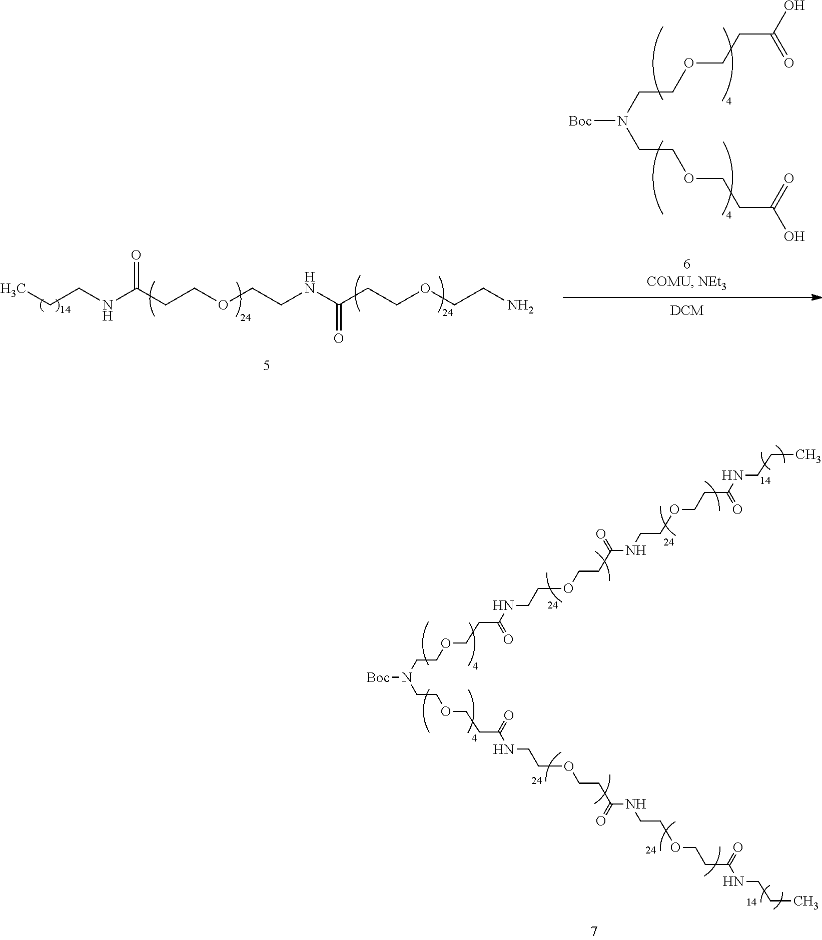

N-Boc-N-Bis-PEG4-Acid (compound 6, 0.0339 g, 0.055 mmol) and COMU (0.0473 g, 0.11 mmol) were dissolved in DCM (3 mL) and NEt₃ (0.167 mL, 1.20 mmol) was added. The resulting solution was stirred for 10 minutes. After 10 minutes compound 5 (0.30 g, 0.12 mmol) was added to the solution of compound 6. The resulting solution was stirred for 1 hour. The reaction mixture was concentrated and loaded directly onto a silica gel column for purification. Crude product was purified by silica gel chromatography 0-20% MeOH in DCM. Compound 7 began eluting with 6% MeOH in DCM. The majority of pure compound 7 eluted with 12% MeOH in DCM. Pure fractions of compound 7 were combined to yield 264 mg (86% yield) as an off-white solid.

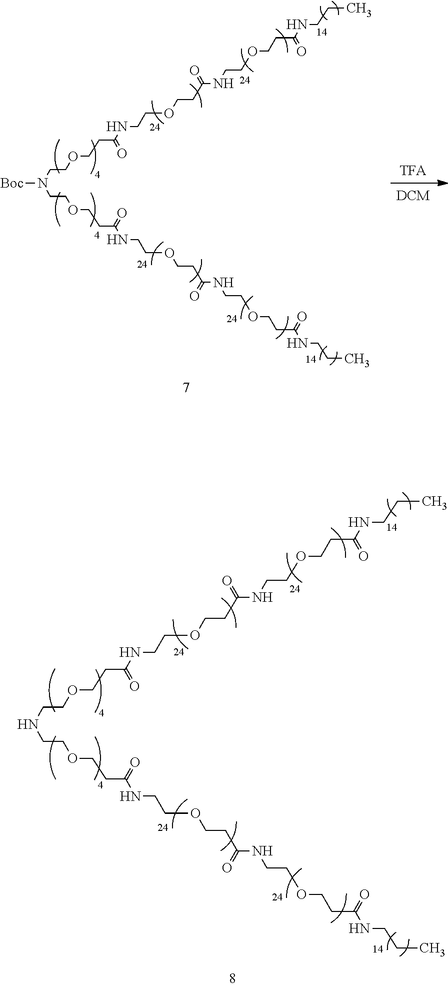

Compound 7 (100 mg, 0.041 mmol) was dissolved in DCM (2 mL) and TFA (1 mL, 8.64 mmol) was added. The reaction mixture was stirred for 2 hours and checked by LC-MS. The reaction mixture was quenched with sat. NaHCO_{3(aq)} and diluted with DCM. The layers were separated and the organic layer was washed with sat. NaCl(aq) (20 mL), dried over Na₂SO₄ and concentrated to yield 0.09 g of compound 8 as a light yellow solid (86% yield). Compound 8 was used directly in the next step without further purification.

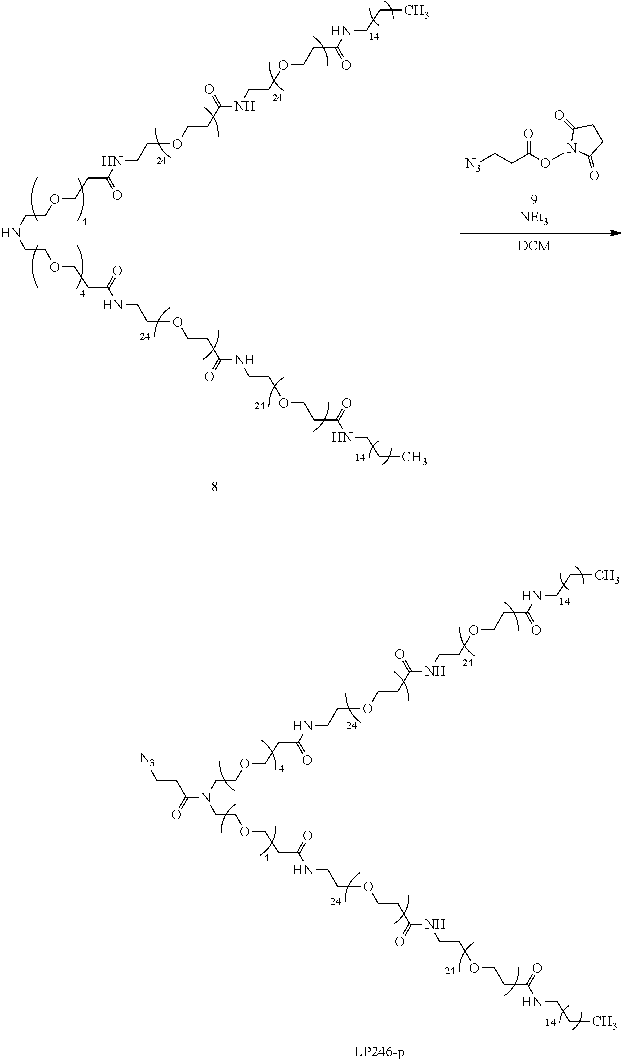

Compound 8 (0.090 g, 0.016 mmol) was dissolved in DCM (3 mL) and NEt₃ (22.9 μL, 0.164 mmol) was added followed by the addition of 3-azido propionate NHS-ester (compound 9, 0.0174 g, 0.082 mmol). The reaction mixture was stirred for 4 hours and checked by LC-MS. The reaction mixture was concentrated and loaded directly onto a silica gel column for purification. Crude product was purified by silica gel chromatography (4 g Redisep Gold® column available from Teledyne Isco) 0-20% MeOH in DCM. LP246-p eluted with 16% MeOH in DCM. Pure fractions of LP246-p were combined to yield 0.019 g of an off-white solid (20.7% yield).

### Synthesis of LP247-p

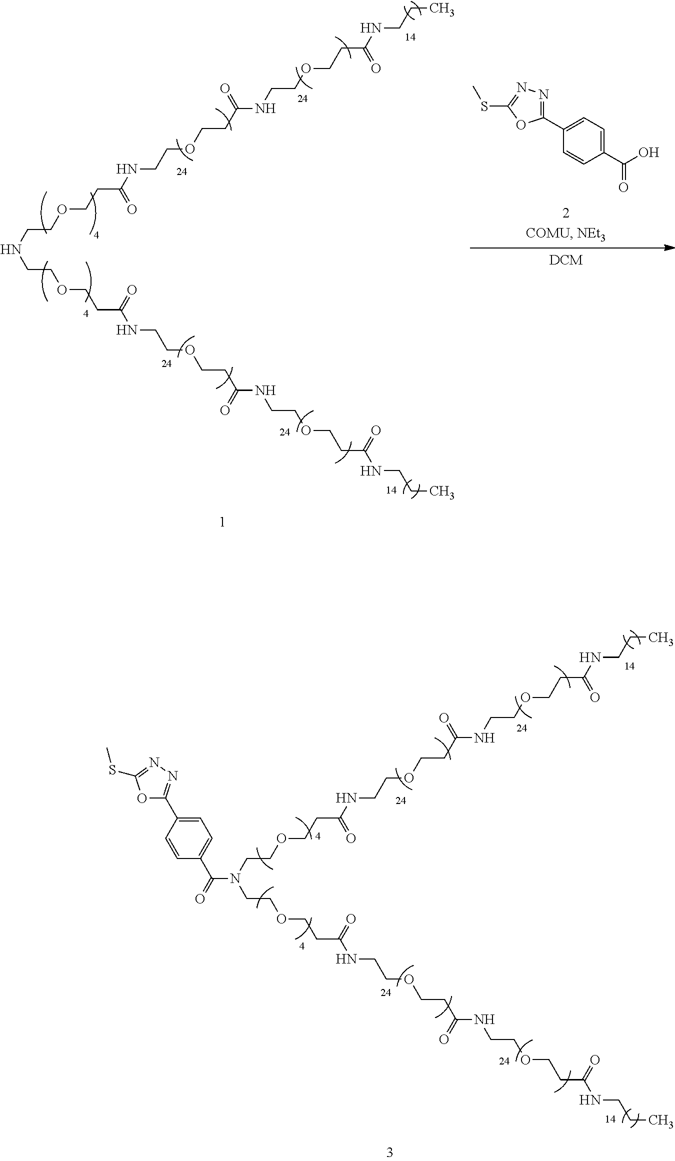

Compound 2 (11.2 mg, 0.047 mmol) and COMU (20 mg, 0.047 mmol) were dissolved in DCM (3 mL) and NEt₃ (16.7 μL, 0.12 mmol) was added. The resulting solution was stirred for 10 minutes. After 10 minutes, a solution of compound 1 (130 mg, 0.024 mmol, compound 8 from synthesis of LP246-p) in DCM (2 mL) was added to the solution of compound 2/COMU. The resulting solution was stirred for 1 hour and checked by LC-MS. The reaction mixture was quenched with 5 mL of water and stirred for 5 minutes. The layers were separated, and the organic layer was washed with 1 M HCl(aq) (1×15 mL), sat. NaHCO₃(aq) (3×20 mL), sat. NaCl(aq) (20 mL), dried over Na₂SO₄ and concentrated to yield a clear liquid. Crude product was purified by silica gel chromatography (4 g Redisep Gold® column available from Teledyne Isco) 0-20% MeOH in DCM. Compound 3 eluted with 12% MeOH in DCM. Pure fractions of compound 3 were combined to yield 0.086 g (63.6% yield) as an off white solid.

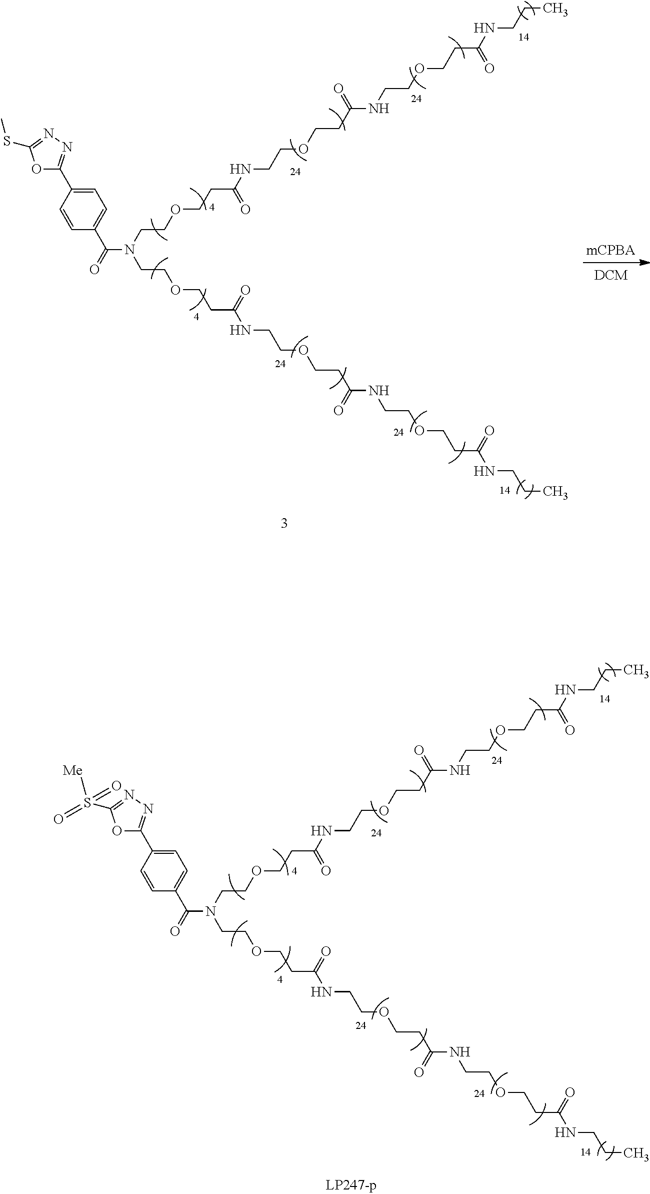

Compound 3 (0.086 g, 0.015 mmol) was dissolved in DCM (3 mL) and mCPBA (0.0131 g, 0.076 mmol) was added. The resulting solution was stirred overnight. The reaction mixture was concentrated and loaded directly onto a silica gel column. Crude product was purified by silica gel chromatography (4 g Redisep Gold® column available from Teledyne Isco) 0-20% MeOH in DCM. LP247-p eluted with 12% MeOH in DCM. Pure fractions of LP247-p were combined to yield 0.041 mg (47.4% yield) as an off white solid.

### Synthesis of LP339-p

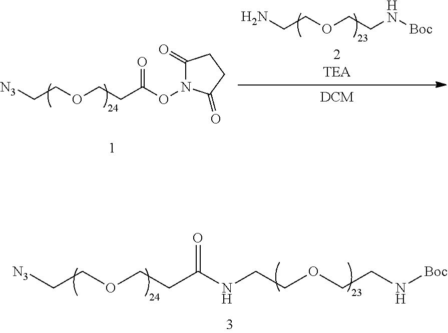

Boc-amido-PEG23-amine 2 (8.00 g, 6.82 mmol) was dissolved in DCM (250 mL) and triethylamine (2.85 mL, 20.45 mmol) was added, followed by azido-PEG24-NHS Ester 1 (9.95 g, 7.84 mmol). The reaction mixture was stirred at room temperature. After 2 hours no starting material remained as determined by LC-MS. The reaction mixture was concentrated and loaded directly onto a silica gel column for purification. The crude product was purified by silica gel chromatography 2% MeOH:98% DCM to 20% MeOH:80% DCM. Fractions containing the product were combined to yield 14.3 grams (90% yield) of compound 3 as a white solid.

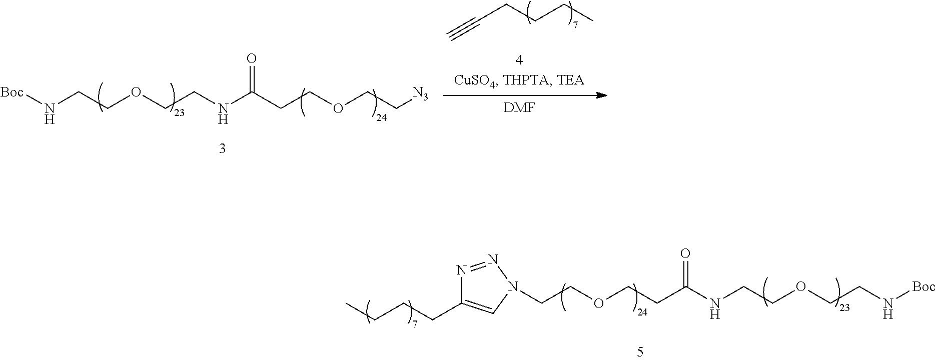

N-Boc-PEG23-Amido-PEG24-Azide 3 (10.0 g, 4.296 mmol), 1-octadecyne 4 (1.183 g, 4.726 mmol), copper sulfate pentahydrate (0.268 g, 1.074 mmol), tris((1-hydroxy-propyl-1H-1,2,3-triazole-4-yl)methyl)amine (THPTA) (0.653 g, 1.504 mmol), and sodium ascorbate (1.872 g, 9.451 mmol) were dissolved in DMF (500 mL) and triethylamine (0.290 mL, 2.148 mmol) was added. The reaction mixture was heated to 60° C. After 2 hours, no starting material was observed by LC-MS. The reaction mixture was concentrated, and the residue was diluted with dichloromethane and filtered through a fritted funnel. The filtrate was concentrated and loaded directly onto a silica gel column for purification. The crude product was purified by silica gel chromatography 0% MeOH:100% DCM to 20% MeOH:80% DCM. The product eluted at 8% MeOH/92% DCM. Pure fractions were combined to yield 9.5 g (86% yield) of compound 5 as a light yellow solid.

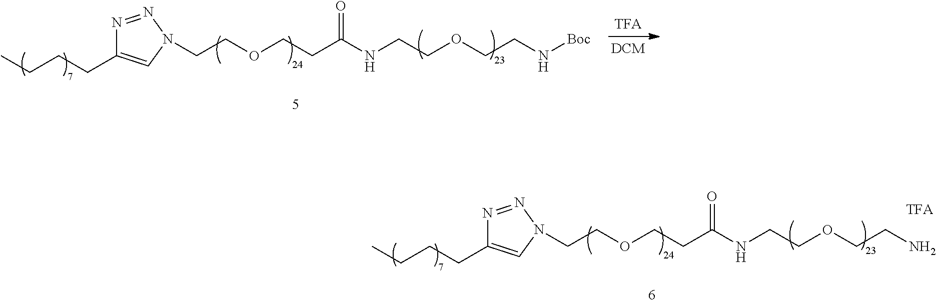

N-Boc-PEG₂₃-Amido-PEG24-Triazole-C₁₆ 5 (0.358 g, 0.139 mmol) was dissolved in DCM (4 mL) and trifluoroacetic acid (0.9 mL, 11.8 mmol) was added. After 1 hour, no starting material was observed by LC-MS. The reaction mixture was concentrated and dried under vacuum for several hours to yield 0.325 mg (90.9% yield) of compound 6 as a light yellow solid. The product was used directly in the next reaction without further purification.

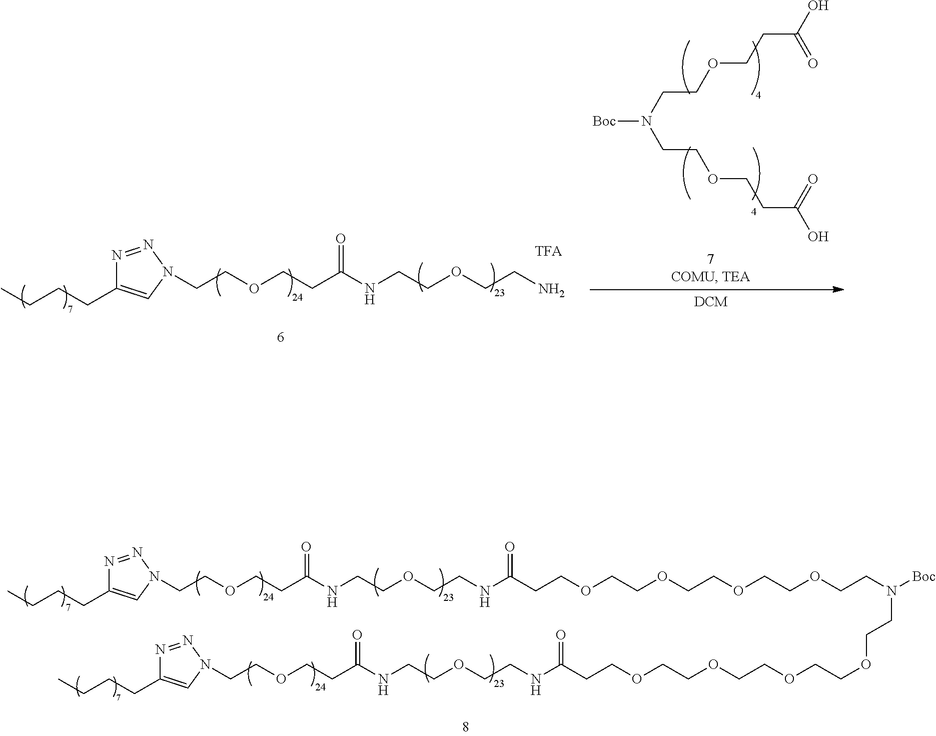

N-Boc-N-Bis-PEG4-Acid 7 (0.0372 g, 0.061 mmol) and COMU (0.052 g, 0.121 mmol) were dissolved in DCM (5 mL) and TEA (0.395 mL, 2.84 mmol) was added. The resulting solution was stirred for 10 minutes. In a separate vial, a solution of the TFA salt of Amino-PEG₂₃-amido-PEG₂₄-triazole-C₁₆ 6 (0.325 g, 0.126 mmol) in DCM (5 mL) and TEA (0.5 mL, 3.60 mmol) was stirred. The solution of N-Boc-N-Bis-PEG₄-Acid 7 was added to the solution of Amino-PEG₂₃-amido-PEG₂₄-triazole-C₁₆ 6. The reaction mixture was stirred overnight. The reaction mixture was concentrated and loaded directly onto a silica gel column for purification. The crude product was purified by silica gel chromatography 4% MeOH:96% DCM to 20% MeOH:80% DCM. Pure fractions were combined to yield 89 mg (26.5% yield) of compound 8 as a light yellow solid.

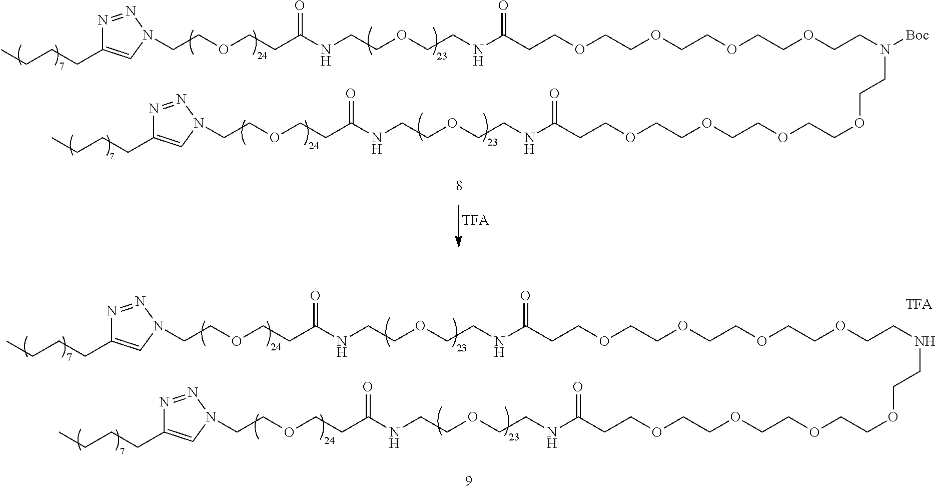

N-Boc-bis-PEG₄-Amido-PEG₂₃-amido-PEG₂₄-Triazole-C₁₆ 8 (5.9 g, 1.066 mmol) was dissolved in DCM (100 mL) and TFA (20 mL, 262.3 mmol) was added. After 2 hours, no starting material was observed by LC-MS. The reaction mixture was concentrated to afford compound 9 as a thick yellow liquid. Compound 9 was used directly in the next step without further purification.

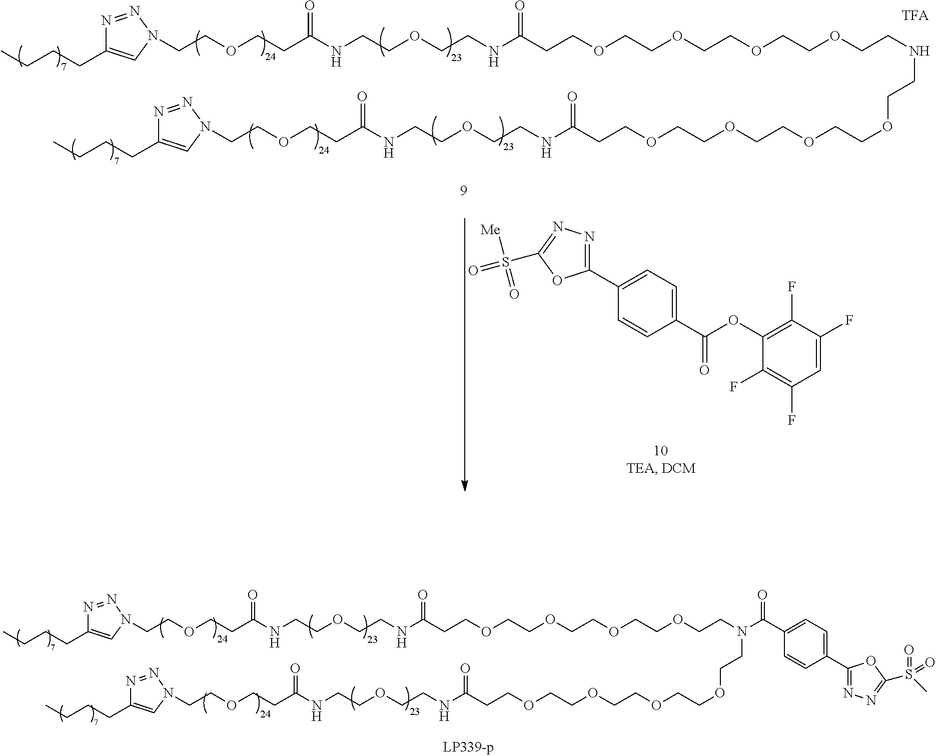

The TFA salt of amino-bis-PEG₄-Amido-PEG₂₃-amido-PEG₂₄-Triazole-C₁₆ 9 (5.89 g, 1.066 mmol) was dissolved in THF (100 mL) and TEA (1.5 mL, 10.66 mmol) and TFP-sulfone 10 (1.33 g, 3.20 mmol) was added. After 22 hours, LC-MS indicated 95% conversion to the product. The reaction mixture was concentrated, resuspended in toluene, and concentrated again prior to purification. The crude product was purified by silica gel chromatography 5% MeOH:95% DCM to 20% MeOH:80% DCM. The product eluted with 8% MeOH:92% DCM. Pure fractions were combined and yielded 3.000 g (49.5% yield) of LP339-p as a beige solid.

### Synthesis of LP340-p

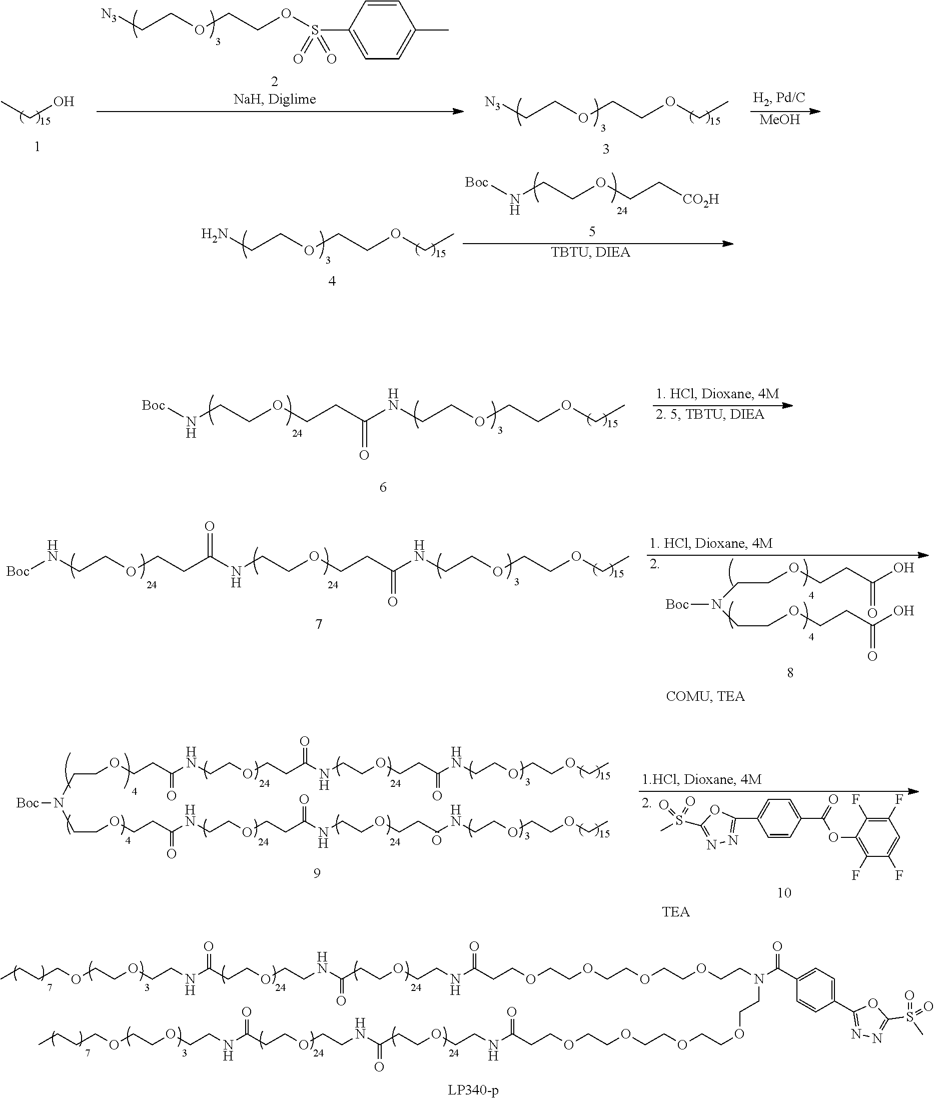

Sodium hydride, 60% dispersion in mineral oil (1.93 g, 48.21 mmol) was loaded in a dry 1 L round bottom flask, washed with MTBE and suspended in anhyd dioxane (200 mL). Hexadecanol 1 (11.2 g, 46.2 mmol) was added dry and stirred for 1 hour at 50° C. Peg3-tosylate 2 (15 g, 40.17 mmol) was added, and the reaction mixture was heated for 17 hours at 105° C. The reaction mixture was cooled in an ice bath and H₂O (125 mL) was added. The mixture was extracted with MTBE, and the organic layer was washed with H₂O, brine, and dried over Na₂SO₄. Compound 3 was purified on CombiFlash® using 220 g SiO₂ column, eluent: solvent A—hexane, solvent B—EtOAc; B=0-30%, 50 min. Yield, 11.1 g, 64%. Calculated MW 443.67 Found: MS (ES, pos): 444.67 [M+H]+, 461.5 [M+NH₄]⁺, 466.54 [M+Na]⁺.

Azide 3 (11.1 g, 25 mmol) was stirred with Pd/C, 10% (1 g) in MeOH (70 mL) under a hydrogen atmosphere for 17 hours at 1 atmosphere. The reaction mixture was filtered, concentrated, and dried under vacuum. Compound 4 was purified on CombiFlash® using 80 g SiO₂ column, eluent: solvent A—DCM, solvent B—20% MeOH in DCM; B=0-50% in 50 min. Yield 4.66 g. Calculated: MW 417.7. Found: MS (ES, pos): 418.1 [M+H]+.

TBTU (4.8 g, 14.9 mmol) was added to a suspension of amine 4 (5.94 g, 14.2 mmol), Boc-(Peg 24)-acid 5 (16.95 g, 13.6 mmol), and DIEA (7.1 mL, 40.8 mmol) in DMF (100 mL). The reaction mixture was stirred for 3 hours, concentrated, and the residual DMF was removed by 3 co-evaporations with toluene. Crude compound 6 was dissolved in CHCl₃ (500 mL), washed with 1% HCl, NaHCO₃, brine, dried over Na₂SO₄, and used directly in the next step without further purification. Calculated: MW 1646.14. Found: MS (ES, pos): 1646.99 [M+H]⁺, 1664.99 [M+NH₄]⁺.

Compound 6 (10.14 g, 6.16 mmol) was stirred in a 4M HCl dioxane solution (45 mL) for 50 minutes. The reaction mixture was concentrated and the residue was dried by 2 co-evaporations with toluene. The resultant deprotected Peg-amine hydrochloride was dissolved in DMF (60 ml), then DIEA (4.29 mL, 24.6 mmol) and acid 5 (7.674 g, 6.157 mmol) were added, followed by TBTU (2.175 g, 6.77 mmol). The reaction mixture was stirred for 4 hours. The reaction mixture was concentrated and the residue was dried by 3 co-evaporations with toluene. The product, compound 7, was dissolved in CHCl₃ (500 mL), washed with 1% HCl, NaHCO₃, brine, and dried over Na₂SO₄. Compound 7 was used directly in the next step without further purification. Calculated: MW 2774.49. Found: MS (ES, pos): 1405.24 [M+2NH₄]²+1397.20 [M+H+Na]²+1388.67 [M+2H]²⁺.

Compound 7 (15.22 g, 5.49 mmol) was stirred in a 4M HCl dioxane solution (55 mL) for 50 minutes. The reaction mixture was concentrated and the residue was dried by 2 co-evaporations with toluene. The resultant deprotected Peg-amine hydrochloride was dissolved in DCM (100 mL). Boc-amino-bis(Peg4-acid) 8 (1.68 g, 2.74 mmol) was stirred in DCM (15 mL) with TEA (2.2 mL, 15.8 mmol) and COMU (2.47 g, 5.76 mmol) for 3 minutes, and then added to the solution of the deprotected Peg-amine hydrochloride. The reaction mixture was stirred for 3 hours and the solvent was removed. The residue was dissolved in chloroform (300 mL), washed with 1% HCl, NaHCO₃, brine, and dried over Na₂SO₄. Compound 9 was purified on CombiFlash® using SiO₂ column (220 g), eluent solvent A—DCM, solvent B—20% MeOH in DCM; B=0-100% in 50 min. Yield 9.75 g, (60%). Calculated: MW 5926.42. Found: MS (ES, pos): 1483.26 [M+3H+NH₄]⁴⁺, 1458.53.74 [M+4H]⁴⁺, 1186.91 [M+5H]⁺⁵.

Compound 9 (9.75 g, 1.644 mmol) was stirred in a 4M HCl dioxane solution (60 mL) for 50 minutes. The reaction mixture was concentrated and the residue was dried by 2 co-evaporations with toluene. The resultant amine hydrochloride was dissolved in THF (150 mL) and TEA was added (1.38 mL, 9.86 mmol), followed by sulfone-TFP ester 10 (1.711 g, 4.11 mmol). The reaction mixture was stirred for 16 hours, and the solvent was removed under vacuum. The residue was dissolved in chloroform (300 mL), washed with 1% HCl, brine, and dried over Na₂SO₄. LP340-p was purified on CombiFlash® using SiO₂ column (120 g), eluent solvent A—DCM, solvent B—20% MeOH in DCM; B=0-100% in 60 min. Yield 7.58 g, (75%). Calculated: MW 6077.54. Found: MS (ES, pos): 1534.03 [M+H+Na+2NH₄]⁴⁺, 1227.47 [M+2H+Na+2NH₄]⁵⁺.

### Synthesis of LP357-p

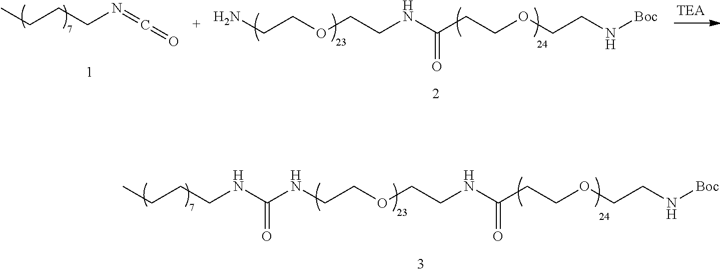

Boc-PEG₄₇-NH2 2 (1 g, 0.435 mmol, 1.0 equiv) was dissolved in 20 mL DCM. Hexadecyl isocyanate 1 (140 mg, 0.522 mmol, 1.2 equiv) and TEA (2.0 equiv) were added and the reaction mixture was stirred at room temperature for 12 hours. DCM was removed and compound 3 0.967 g (86.5%) was purified via 24 g column purification using 0-20% MeOH/DCM as the mobile phase.

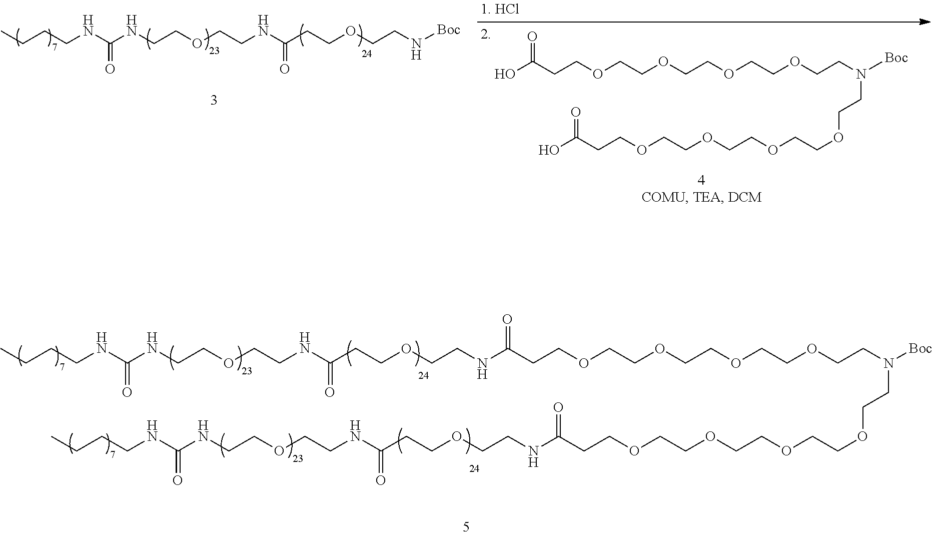

Compound 3 (0.967 g, 0.376 mmol) was dissolved in 15 mL of 4N HCl/dioxane and stirred at room temperature for 1 hour. The HCl/dioxane was removed and the resultant deprotected amine was dissolved in DCM. Compound 4 (110 mg, 0.179 mmol), COMU (169 mg, 0.394 mmol) and TEA (10.0 equiv) were added and the reaction mixture was stirred at room temperature overnight. The solvent was removed under vacuum. Compound 5 (0.8 g, 80.9% yield) was purified by a 24 g column using 0-20% MeOH/DCM as the mobile phase.

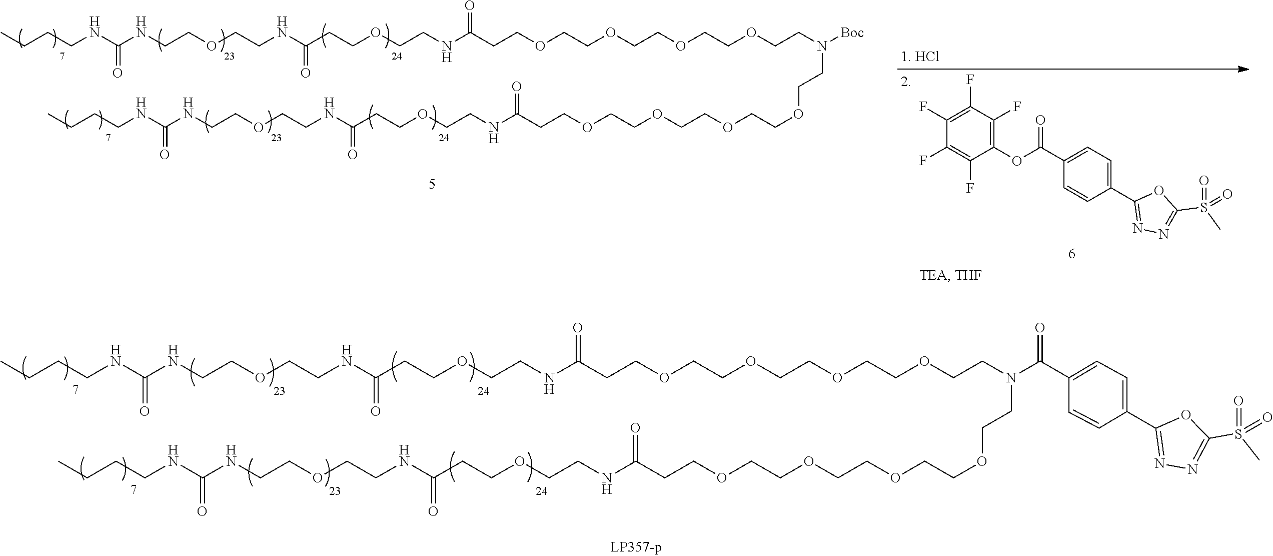

Compound 5 (0.95 g, 0.172 mmol) was dissolved in 15 mL of 4N HCl/Dioxane and stirred at room temperature for 1 hour. The HCl/dioxane was removed under vacuum. The resulting deprotected amine was dissolved in THF, then compound 6 (0.15 g, 0.345 mmol) and TEA (10.0 equiv) were added. The reaction mixture was stirred at room temperature overnight. The solvent was removed under vacuum. LP357-p (0.6 g, 61%) was purified by a 24 g column using 0-20% MeOH/DCM as the mobile phase.

### Synthesis of LP358-p

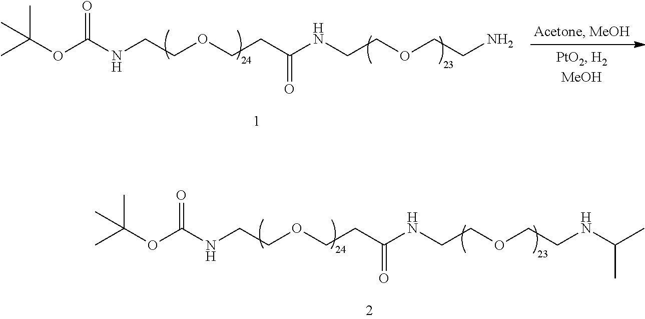

PtO₂ (0.3986 g) was added to a solution of compound 1 (4.00 g) in anhydrous MeOH and acetone. The reaction mixture was stirred for two days under a hydrogen atmosphere. The platinum catalyst was filtered out using Celite® and silica. The solution was then concentrated under vacuum to afford compound 2 which was used directly in the next step without purification. Yield: 3.99 g.

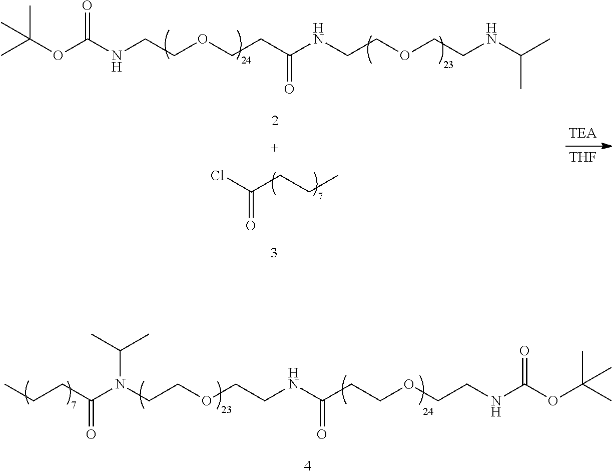

Compound 2 (4.07 g) was added to a solution of compound 3 (0.53 g) and TEA (0.53 g) in THF. The reaction mixture was stirred until full conversion of 2 was observed by LC-MS and/or TLC. The reaction mixture was quenched with MeOH. The crude product was purified on a CombiFlash® system via a DCM liquid-load (80 g column, DCM (A) to 20% MeOH (B) solvent system, gradient: 5% B to 100% B over 60 min). Compound 4 eluted at 25% B. Yield: 2.92 g.

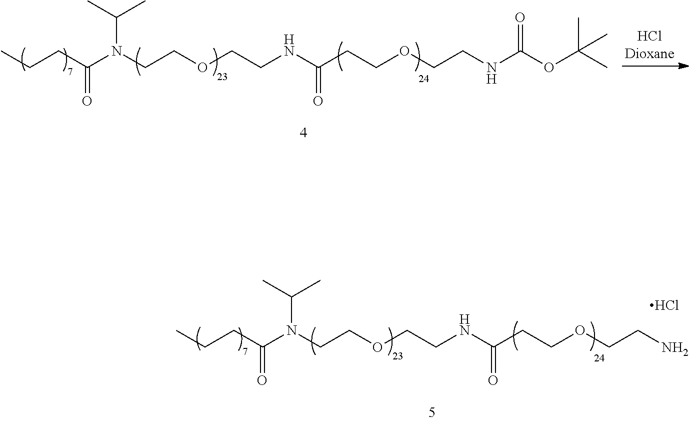

Compound 4 (2.92 g) was dissolved in a solution of HCl in Dioxane (4M) (24.9 mL) at room temperature. The reaction mixture was stirred until full conversion of compound 4 was observed via LCMS. The reaction mixture was concentrated under vacuum to afford compound 5 as a white powder. Compound 5 was used directly in the next step without further purification.

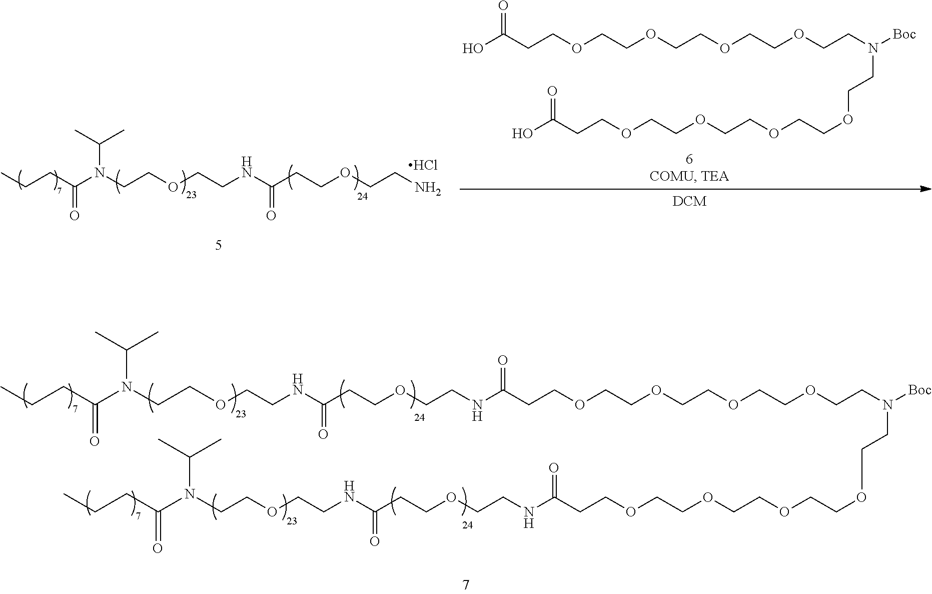

Compounds 6 (0.32 g) and 5 (2.81 g), COMU (1.07 g), and TEA (2.08 mL) were stirred in DCM at room temperature overnight. The pH was monitored to ensure that the HCl was neutralized and that the reaction mixture remained basic. The reaction mixture was washed with 1N HCl, saturated NaHCO₃, and brine, and the DCM was removed under vacuum. Compound 7 was purified via an 80 g column (Solvent system: DCM (A) and 20% MeOH (B), gradient: 5% B for 5 min, 5% B to 100% B over 60 min). Compound 7 eluted at 45% B. Yield 2.26 g.

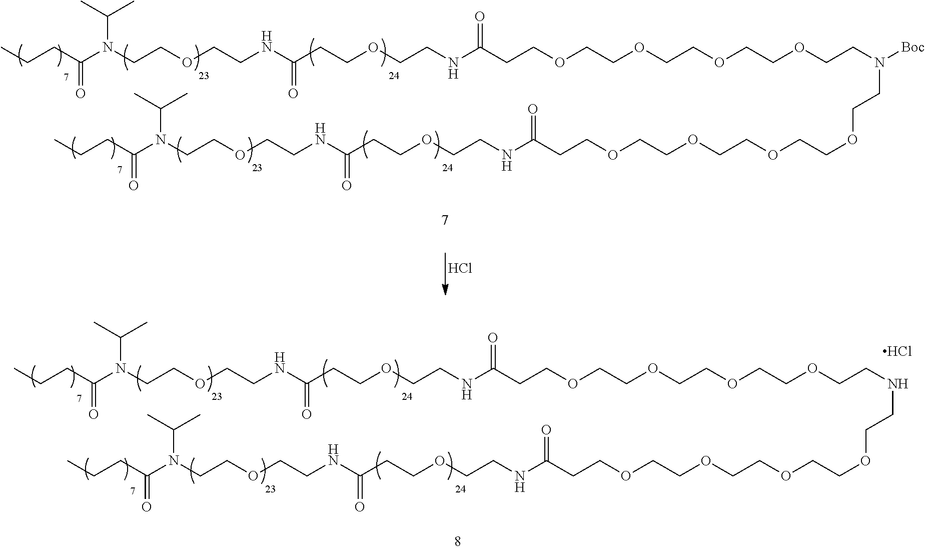

Compound 7 (2.26 g) was dissolved in a solution of HCl in Dioxane (4M) (25.5 mL) at room temperature. The reaction mixture was stirred until full conversion of compound 7 was observed via LCMS. The reaction mixture was concentrated under vacuum to afford compound 8 as a white powder. Compound 8 was used directly in the next step without further purification.

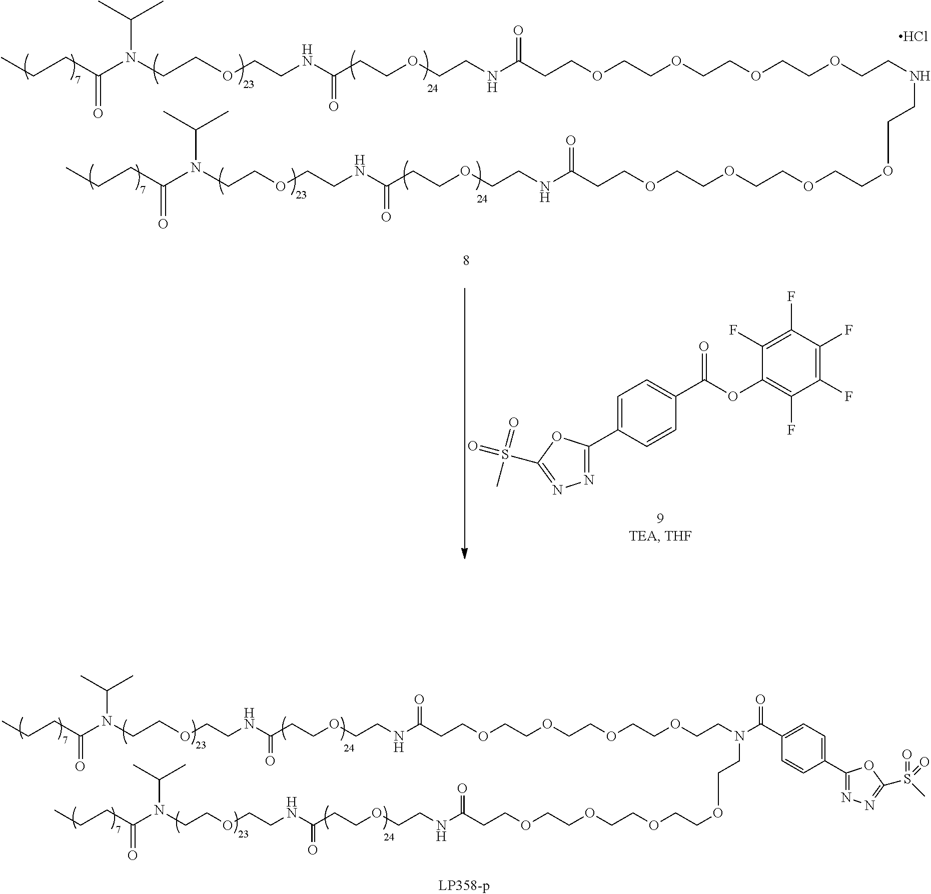

Compound 8 (2.22 g) and TEA (1.42 mL) were dissolved in 50 mL of anhydrous THF, and compound 9 (0.35 g) was added. The reaction mixture was stirred for 12 hours. The reaction mixture was concentrated and the crude LP358-p was purified by silica in two parts (12 and 24 gram columns) using two solvent systems (EtOAc/Hexanes followed by MeOH/DCM. 1st gradient (EtOAc/Hexanes): 0% B for 3 minutes, 0% B to 100% B over 10 min. 2nd gradient (DCM/MeOH): 5% B for 5 minutes, 15% B for 5 minutes, 15% B to 100% B over 20 min.). The product, LP358-p, eluted at 30% B during the second gradient. The sulfone reagent (i.e., compound 9) was recovered during the first gradient. Yield 1.92 g.

### Example 5. Conjugation of Linking Groups and Targeting Ligands to RNAi Agents

A. Conjugation of Activated Ester Linking Groups

The following procedure was used to conjugate linking groups having the structure of DBCO-NHS or L₁-L₁₀ as shown in Table 22 above to an RNAi agent with an amine-functionalized sense strand, such as C6-NH2, NH2-C6, or (NH2-C6)s, as shown in Table 22, above. An annealed RNAi Agent dried by lyophilization was dissolved in DMSO and 10% water (v/v %) at 25 mg/mL. Then 50-100 equivalents of TEA and 3 equivalents of activated ester linker were added to the solution. The solution was allowed to react for 1-2 hours, while monitored by RP-HPLC-MS (mobile phase A 100 mM HFIP, 14 mM TEA; mobile phase B: acetonitrile on an Waters™ XBridge C18 column, Waters Corp.)

The product was then precipitated by adding 12 mL acetonitrile and 0.4 mL PBS and centrifuging the solid to a pellet. The pellet was then re-dissolved in 0.4 mL of 1×PBS and 12 mL of acetonitrile. The resulting pellet was dried on high vacuum for one hour.

B. Conjugation of Targeting Ligands to Propargyl Linkers

Either prior to or after annealing, the 5′ or 3′ tridentate alkyne functionalized sense strand is conjugated to the αvβ6 Integrin Ligands. The following example describes the conjugation of αvβ6 integrin ligands to the annealed duplex: Stock solutions of 0.5M Tris(3-hydroxypropyltriazolylmethyl)amine (THPTA), 0.5M of Cu(II) sulfate pentahydrate (Cu(II)SO₄ 5 H₂O) and 2M solution of sodium ascorbate were prepared in deionized water. A 75 mg/mL solution in DMSO of αvβ6 integrin ligand was made. In a 1.5 mL centrifuge tube containing tri-alkyne functionalized duplex (3 mg, 75 μL, 40 mg/mL in deionized water, approximately 15,000 g/mol), 25 μL of 1M Hepes pH 8.5 buffer is added. After vortexing, 35 μL of DMSO was added and the solution is vortexed. αvβ6 integrin ligand was added to the reaction (6 eq/duplex, 2 eq/alkyne, approximately 15 μL) and the solution is vortexed. Using pH paper, pH was checked and confirmed to be pH approximately 8. In a separate 1.5 mL centrifuge tube, 50 μL of 0.5M THPTA was mixed with 10 uL of 0.5M Cu(II)SO₄·5 H₂O, vortexed, and incubated at room temp for 5 min. After 5 min, THPTA/Cu solution (7.2 μL, 6 eq 5:1 THPTA:Cu) was added to the reaction vial, and vortexed. Immediately afterwards, 2M ascorbate (5 μL, 50 eq per duplex, 16.7 per alkyne) was added to the reaction vial and vortexed. Once the reaction was complete (typically complete in 0.5-1 h), the reaction mixture was immediately purified by non-denaturing anion exchange chromatography.

### Example 6. Conjugation of Lipid PK/PD Modulator Precursors

Either prior to or after annealing and prior to or after conjugation of one or more targeting ligands, one or more lipid PK/PD modulator precursors can be linked to the RNAi agents disclosed herein. The following describes the general conjugation process used to link lipid PK/PD modulator precursors to the constructs set forth in the Examples depicted herein.

A. Conjugation of a Maleimide-Containing Lipid PK/PD Modulator Precursor

The following describes the general process used to link a maleimide-containing lipid PK/PD modulator precursor to the (C6-SS-C6) or (6-SS-6) functionalized sense strand of an RNAi agent by undertaking a dithiothreitol reduction of disulfide followed by a thiol-Michael Addition of the respective maleimide-containing lipid PK/PD modulator precursor: In a vial, functionalized sense strand was dissolved at 50 mg/mL in sterilized water. Then 20 equivalents of each of 0.1M Hepes pH 8.5 buffer and dithiothreitol were added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the maleimide-containing lipid PK/PD modulator precursor was added at 1.5 equivalents. The mixture was allowed to react for 30 minutes. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

B. Conjugation of a Sulfone-Containing Lipid PK/PD Modulator Precursor

In a vial, functionalized sense strand was dissolved at 50 mg/mL in sterilized water. Then 20 equivalents of each of 0.1 M Hepes pH 8.5 buffer and dithiothreitol are added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the sulfone-containing lipid PK/PD modulator precursor was added at 1.5 equivalents. The vial was purged with N₂, and heated to 40° C. while stirring. The mixture was allowed to react for one hour. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

C. Conjugation of an Azide-Containing Lipid PK/PD Modulator Precursor

One molar equivalent of TG-TBTA resin loaded with Cu(I) was weighed into a glass vial. The vial was purged with N₂ for 15 minutes. Then, functionalized sense strand was dissolved in a separate vial in sterilized water at a concentration of 100 mg/mL. Then two equivalents of the azide-containing lipid PK/PD modulator precursor (50 mg/mL in DMF) is added to the vial. Then TEA, DMF and water are added until the final reaction conditions are 33 mM TEA, 60% DMF, and 20 mg/mL of the conjugated product. The solution was then transferred to the vial with resin via a syringe. The N₂ purge was removed and the vial was sealed and moved to a stir plate at 40° C. The mixture was allowed to react for 16 hours. The resin was filtered off using a 0.45 μm filter.

The product was purified using AEX purification (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile solid phase TSKgel-30; 1.5 cm×10 cm.) The acetonitrile was removed using a rotary evaporator, and desalted with a 3K spin column using 2×10 mL exchanges with sterilized water. The solid product was dried using lyophilization and stored for later use.

D. Conjugation of an Alkyne-Containing Lipid PK/PD Modulator Precursor

The following describes the general process used to link an activated alkyne-containing lipid PK/PD modulator precursor to the (C6-SS-C6) or (6-SS-6) functionalized sense strand of an RNAi agent by undertaking a dithiothreitol reduction of disulfide followed by addition to an alkyne-containing PK/PD modulator precursor: In a vial, 10 mg of siRNA comprising the (C6-SS-C6) or (6-SS-6) functionalized sense strand was dissolved at 50 mg/mL in sterilized water. Then 20 equivalents of each of 0.1M Hepes pH 8.5 buffer and dithiothreitol (1M in sterilized water) were added. The mixture was allowed to react for one hour, then purified on Waters™ XBridge BEH C4 Column using a mobile phase A of 100 mM HFIP, 14 mM, and TEA, and a mobile phase B of Acetonitrile using the following formula, wherein % B indicates the amount of mobile phase B while the remainder is mobile phase A.

| Time | % B |
|---|---|
| 0 | 3 |
| 8 | 70 |
| 10 | 90 |
| 11 | 90 |
| 11.1 | 3 |
| 13 | 3 |

The product was precipitated once by adding 12 mL of acetonitrile and 0.4 mL 1×PBS, and the resulting solid was centrifuged into a pellet. The pellet was re-dissolved in 0.4 mL 1×PBS and 12 mL of acetonitrile. The pellet was dried on high vacuum for one hour.

The pellet was brought up in a vial a 70/30 mixture of DMSO/water at a solids concentration of 30 mg/mL. Then, the alkyne-containing lipid PK/PD modulator precursor was added at 2 equivalents relative to siRNA. Then 10 equivalents of TEA was added. The vial was purged using N₂, and the reaction mixture was heated to 40° C. while stirring. The mixture was allowed to react for one hour. The product was purified using anion-exchange HPLC using a TSKgel-30 (available from Tosoh Biosceinces) packed column, 1.5 cm×10 cm, using a mobile phase A of 25 mM TRIS pH=7.2, 1 mM EDTA, 50% Acetonitrile, and a mobile phase B of 25 mM TRIS pH=7.2, 1 mM EDTA, 500 mM NaBr, 50% Acetonitrile using the following formula, wherein % B indicates the amount of mobile phase B while the remainder is mobile phase A.

| Time | % B |
|---|---|
| 4 | 10 |
| 7 | 80 |
| 10.5 | 80 |
| 11 | 10 |
| 14 | 10 |

The fractions containing the product were collected, and acetonitrile was removed using a rotary evaporator. The product was desalted with a 3K spin column, using 2×10 mL exchanges with sterilized water. The product was then dried using lyophilization and stored for later use.

### Example 7. In Vivo Administration of RNAi Triggers Targeting Mstn in Mice

Myostatin RNAi agents that included a sense strand and an antisense strand were synthesized according to phosphoramidite technology on solid phase in accordance with general procedures known in the art and commonly used in oligonucleotide synthesis, as set forth in Example 1 herein. RNAi agents used in this and following Examples have the structure as indicated in Table 24, below.

TABLE 24
Duplexes used in the Following Examples

| | | SEQ |
|---|---|---|
| Duplex | | ID |
| Name | Structure (5′->3′) | NO |
| AD06569 | AS: cPrpusGfsusUfaCf | 1 |
| | agcaaGfaUfcAfuGfaCfs | |
| | c | |
| | SS: (NH2-C6)s(invAb) | 2 |
| | sggucaugaUfCfUfugcug | |
| | uaacas(invAb)(C6-SS- | |
| | C6)dT | |
| AD07724 | AS: cPrpusGfsusUfaCf | 3 |
| | agcaaGfaUfcAfuGfaCfs | |
| | c | |
| | SS: (NH2-C6)s(invAb) | 4 |
| | sggucaugaUfCfUfugcug | |
| | uaacas(invAb)uAlkdT | |
| AD07909 | AS: cPrpusGfsusUfaCf | 5 |
| | agcaaGfaUfcAfuGfaCfs | |
| | c | |
| | SS: (NH2-C6)s(invAb) | 6 |
| | sggucaugaUfCfUfugcug | |
| | uaacas(invAb)uAlk | |
| AD07910 | AS: cPrpusGfsusUfaCf | 7 |
| | agcaaGfaUfcAfuGfaCfs | |
| | c | |
| | SS: (NH2-C6)s(invAb) | 8 |
| | sggucaugaUfCfUfugcug | |
| | uaacaAlks(invAb) | |
| AD08257 | AS: cPrpusGfuUfacagc | 9 |
| | aaGfaUfcsAfsusGfsasC | |
| | fsc | |
| | SS: (NH2-C6)s(invAb) | 10 |
| | sggucaugaUfCfUfugcug | |
| | uaacas(invAb)(LA2) | |

In Table 24 above, AS represents the antisense strand, SS represents the sense strand; a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue (see Table 22); dT represents 2′-deoxythymidine-3′-phosphate; cPrp represents cyclopropyl phosphonate, see Table 22; aAlk represents 2′-O-propargyladenosine-3′-phosphate, see Table 22; cAlk represents 2′-O-propargylcytidine-3′-phosphate, see Table 22; gAlk represents 2′-O-propargylguanosine-3′-phosphate, see Table 22; tAlk represents 2′-O-propargyl-5-methyluridine-3′-phosphate, see Table 22; uAlk represents 2′-O-propargyluridine-3′-phosphate, see Table 22; (C6-SS-C6) see Table 22; (NH2-C6)s see Table 22; and L_{A2} has the structure:

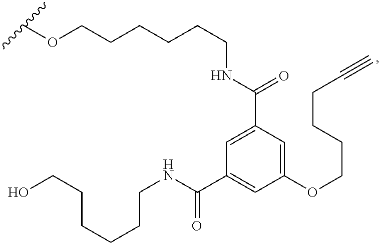 [SMILES: *OCCCCCCNC(=O)c1cc(OCCCCC#C)cc(C(=O)NCCCCCCO)c1]

wherein [symbol] indicates a point of connection to the remainder of the RNAi agent.

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 25
Dosing Groups for mice of Example 7.

| | Compound of Invention | |
|---|---|---|
| Group | Comprising RNAi Agent and Dose | Dosing Regimen |
| 1 | Saline | Single Injection on Day 1 |
| 2 | 2 mpk AD06569-LP1 | Single Injection on Day 1 |
| 3 | 2 mpk SM45b-(L4)-AD06569-LP1b | Single Injection on Day 1 |
| 4 | 2 mpk avb6-Pep1-AD06569-nEm | Single Injection on Day 1 |
| 5 | 2 mpk avb6-Pep1-AD06569-LP1b | Single Injection on Day 1 |
| 6 | 2 mpk avb6-Pep1-AD06569-LP29b | Single Injection on Day 1 |
| 7 | 2 mpk avb6-Pep1-AD06569-LP33b | Single Injection on Day 1 |
| 8 | 2 mpk SM45b-(L4)-AD06569-LP28b | Single Injection on Day 1 |
| 9 | 2 mpk SM45b-(L4)-AD06569-LP56b | Single Injection on Day 1 |
| 10 | 2 mpk SM45b-(L4)-AD06569-LP89b | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the small molecule targeting ligand Compound SM45b or Peptide 1. After completing a conjugation reaction according to Example 5 to the propargyl linker, L₄ (structure as shown in Table 22), the targeting ligand αvβ6 compound 45 had the following structure, referred to in Examples herein as SM45b:

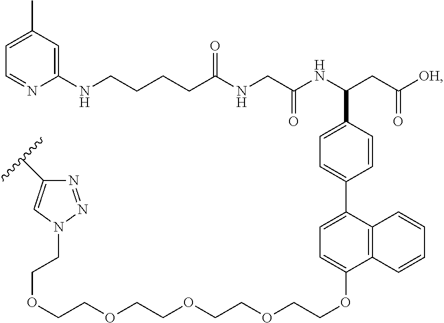 [SMILES: *c1cn(CCOCCOCCOCCOCCOc2ccc(-c3ccc([C@H](CC(=O)O)NC(=O)CNC(=O)CCCCNc4cc(C)ccn4)cc3)c3ccccc23)nn1]

wherein [symbol] indicates a point of connection to the remainder of the RNAi agent. The RNAi agent was also synthesized having a (C6-SS-C6) group on the 3′ end, to facilitate conjugation to a lipid PK/PD modulator precursor.

Groups 2 and 8-10 comprise an αvβ6 integrin ligand SM45 conjugated to the 5′ end of the sense strand using L₄ according to procedures described in Example 5, above. Groups 4-7 comprise an αvβ6 integrin ligand Pep1 conjugated to the 5′ end of the sense strand according to procedures described in Example 5, above. Each of groups 2, 3 and 5-10 comprise a lipid PK/PD modulator, with structures as shown in supra, conjugated to the 3′ end of the sense strand according to procedures described in Example 6, above. Group 4 was conjugated to N-ethylmaleimide (nEm) as a control group.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 26 below.

TABLE 26
Average relative MSTN expression from serum for mice of Example 7.

| | Day 8 | | Day 15 | | Day 22 | |
|---|---|---|---|---|---|---|
| | | Std | | Std | | Std |
| Group | Avg | Dev | Avg | Dev | Avg | Dev |
| 1. Saline | 0.795 | 0.126 | 0.893 | 0.108 | 0.940 | 0.058 |
| 2. AD06569-LP1b | 0.620 | 0.074 | 0.435 | 0.028 | 0.417 | 0.033 |
| 3. SM45b-(L4)-AD06569- | 0.296 | 0.040 | 0.170 | 0.023 | 0.173 | 0.020 |
| LP1b | | | | | | |
| 4. avb6-Pep1-AD06569-nEm | 0.725 | 0.033 | 0.677 | 0.041 | 0.754 | 0.092 |
| 5. avb6-Pep1-AD06569-LP1b | 0.370 | 0.031 | 0.261 | 0.021 | 0.247 | 0.028 |
| 6. avb6-Pep1-AD06569-LP29b | 0.435 | 0.101 | 0.304 | 0.075 | 0.326 | 0.043 |
| 7. avb6-Pep1-AD06569-LP33b | 0.456 | 0.042 | 0.271 | 0.021 | 0.296 | 0.031 |
| 8. SM45b-(L4)-AD06569- | 0.394 | 0.047 | 0.249 | 0.037 | 0.241 | 0.044 |
| LP28b | | | | | | |
| 9. SM45b-(L4)-AD06569- | 0.499 | 0.066 | 0.315 | 0.037 | 0.292 | 0.034 |
| LP56b | | | | | | |
| 10. SM45b-(L4)-AD06569- | 0.310 | 0.034 | 0.218 | 0.056 | 0.173 | 0.025 |
| LP89 | | | | | | |

Tissue collected from the gastrocnemius and triceps was used in a TaqMan assay to determine the relative amounts of MSTN in those tissues. Table 27, below, shows the results of the assay.

TABLE 27
Relative Expression in Triceps and Gastrocnemius in dosing groups of
Example 7.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.105 | 0.118 | 1.000 | 0.092 | 0.101 |
| 2. AD06569-LP1b | 0.171 | 0.067 | 0.110 | 0.175 | 0.041 | 0.053 |
| 3. SM45b-(L4)-AD06569- | 0.089 | 0.009 | 0.010 | 0.063 | 0.007 | 0.008 |
| LP1b | | | | | | |
| 4. avb6-Pep1-AD06569-nEm | 0.462 | 0.041 | 0.046 | 0.446 | 0.063 | 0.073 |
| 5. avb6-Pep1-AD06569-LP1b | 0.118 | 0.020 | 0.023 | 0.099 | 0.017 | 0.021 |
| 6. avb6-Pep1-AD06569-LP29b | 0.174 | 0.034 | 0.042 | 0.126 | 0.045 | 0.070 |
| 7. avb6-Pep1-AD06569-LP33b | 0.132 | 0.035 | 0.049 | 0.119 | 0.026 | 0.033 |
| 8. SM45b-(L4)-AD06569- | 0.110 | 0.022 | 0.028 | 0.088 | 0.014 | 0.016 |
| LP28b | | | | | | |
| 9. SM45b-(L4)-AD06569- | 0.164 | 0.036 | 0.047 | 0.147 | 0.039 | 0.053 |
| LP56b | | | | | | |
| 10. SM45b-(L4)-AD06569- | 0.101 | 0.021 | 0.027 | 0.096 | 0.012 | 0.014 |
| LP89 | | | | | | |

### Example 8. In Vivo Administration of RNAi Triggers Targeting MSTN in Mice

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 28
Dosing Groups for mice of Example 8.

| | Compound of Invention | |
|---|---|---|
| Group | Comprising RNAi Agent and Dose | Dosing Regimen |
| 1 | Saline | Single Injection on Day 1 |
| 2 | 2 mpk SM45b-(L4)-AD06569-LP1 | Single Injection on Day 1 |
| 3 | 2 mpk SM45b-(L4)-AD06569-LP90 | Single Injection on Day 1 |
| 4 | 2 mpk SM45b-(L4)-AD06569-LP94 | Single Injection on Day 1 |
| 5 | 2 mpk SM45b-(L4)-AD06569-LP93 | Single Injection on Day 1 |
| 6 | 2 mpk SM45b-(L4)-AD06569-LP92 | Single Injection on Day 1 |
| 7 | 2 mpk SM45b-(L4)-AD06569-LP91 | Single Injection on Day 1 |
| 8 | 2 mpk SM45b-(L4)-AD06569-LP95 | Single Injection on Day 1 |
| 9 | 2 mpk SM45b-(L4)-AD06569-LP5 | Single Injection on Day 1 |
| 10 | 2 mpk SM45b-(L4)-AD06569-LP87 | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the small molecule targeting ligand Compound 45b. The RNAi agent was also synthesized having a (C6-SS-C6) group on the 3′ end, to facilitate conjugation to a lipid PK/PD modulator precursor.

Groups 2-10 comprise an αvβ6 integrin ligand SM45 conjugated to the 5′ end of the sense strand using linker 4 according to procedures described in Example 5, above. Each of groups 2-10 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 29 below.

TABLE 29
Average relative MSTN expression from serum for mice of Example 8.

| | Day 8 | | Day 15 | | Day 22 | |
|---|---|---|---|---|---|---|
| | | Std | | Std | | Std |
| Group | Avg | Dev | Avg | Dev | Avg | Dev |
| 1. Saline | 1.005 | 0.133 | 1.075 | 0.162 | 1.120 | 0.163 |
| 2. SM45b-(L4)-AD06569- | 0.238 | 0.029 | 0.160 | 0.025 | 0.149 | 0.022 |
| LP1b | | | | | | |
| 3. SM45b-(L4)-AD06569- | 0.642 | 0.062 | 0.612 | 0.041 | 0.614 | 0.021 |
| LP90b | | | | | | |
| 4. SM45b-(L4)-AD06569- | 0.479 | 0.057 | 0.300 | 0.021 | 0.331 | 0.033 |
| LP94b | | | | | | |
| 5. SM45b-(L4)-AD06569- | 0.353 | 0.028 | 0.241 | 0.040 | 0.207 | 0.022 |
| LP93b | | | | | | |
| 6. SM45b-(L4)-AD06569- | 0.385 | 0.021 | 0.279 | 0.044 | 0.242 | 0.009 |
| LP92b | | | | | | |
| 7. SM45b-(L4)-AD06569- | 0.240 | 0.024 | 0.177 | 0.020 | 0.154 | 0.029 |
| LP91b | | | | | | |
| 8. SM45b-(L4)-AD06569- | 0.467 | 0.022 | 0.347 | 0.046 | 0.315 | 0.097 |
| LP95b | | | | | | |
| 9. SM45b-(L4)-AD06569- | 0.447 | 0.032 | 0.411 | 0.031 | 0.371 | 0.030 |
| LP5b | | | | | | |
| 10. SM45b-(L4)-AD06569- | 0.445 | 0.064 | 0.399 | 0.077 | 0.376 | 0.061 |
| LP87b | | | | | | |

determine the relative amounts of MSTN in those tissues. Table 30, below, shows the results of the assay.

TABLE 30
Relative Expression in Triceps and Gastrocnemius in dosing groups of
Example 8.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.237 | 0.311 | 1.000 | 0.070 | 0.075 |
| 2. SM45b-(L4)-AD06569- | 0.069 | 0.016 | 0.020 | 0.103 | 0.013 | 0.015 |
| LP1b | | | | | | |
| 3. SM45b-(L4)-AD06569- | 0.490 | 0.035 | 0.037 | 0.477 | 0.041 | 0.045 |
| LP90b | | | | | | |
| 4. SM45b-(L4)-AD06569- | 0.189 | 0.041 | 0.052 | 0.215 | 0.036 | 0.043 |
| LP94b | | | | | | |
| 5. SM45b-(L4)-AD06569- | 0.097 | 0.013 | 0.016 | 0.142 | 0.006 | 0.006 |
| LP93b | | | | | | |
| 6. SM45b-(L4)-AD06569- | 0.120 | 0.014 | 0.016 | 0.175 | 0.034 | 0.042 |
| LP92b | | | | | | |
| 7. SM45b-(L4)-AD06569- | 0.072 | 0.015 | 0.020 | 0.100 | 0.019 | 0.024 |
| LP91b | | | | | | |
| 8. SM45b-(L4)-AD06569- | 0.271 | 0.030 | 0.034 | 0.277 | 0.047 | 0.057 |
| LP95b | | | | | | |
| 9. SM45b-(L4)-AD06569- | 0.191 | 0.028 | 0.032 | 0.249 | 0.025 | 0.028 |
| LP5b | | | | | | |
| 10. SM45b-(L4)-AD06569- | 0.165 | 0.021 | 0.024 | 0.209 | 0.025 | 0.029 |
| LP87b | | | | | | |

### Example 9. In Vivo Administration of RNAi Triggers Targeting Mstn in Mice

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 31
Dosing Groups for mice of Example 9.

| | Compound of Invention | |
|---|---|---|
| Group | Comprising RNAi Agent and Dose | Dosing Regimen |
| 1 | Saline | Single Injection on Day 1 |
| 2 | 2 mpk Avb6-Pep1-AD06569-LP38b | Single Injection on Day 1 |
| 3 | 2 mpk Avb6-Pep1-AD06569-LP101b | Single Injection on Day 1 |
| 4 | 2 mpk Avb6-Pep1-AD06569-LP41b | Single Injection on Day 1 |
| 5 | 2 mpk Avb6-Pep1-AD06569-LP104b | Single Injection on Day 1 |
| 6 | 2 mpk Avb6-Pep1-AD06569-LP53b | Single Injection on Day 1 |
| 7 | 2 mpk Avb6-Pep1-AD06569-LP43b | Single Injection on Day 1 |
| 8 | 2 mpk Avb6-Pep1-AD06569-LP44b | Single Injection on Day 1 |
| 9 | 2 mpk Avb6-Pep1-AD06569-LP102b | Single Injection on Day 1 |
| 10 | 2 mpk Avb6-Pep1-AD06569-LP103b | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the targeting ligand. The RNAi agent was also synthesized having a (C6-SS-C6) group on the 3′ end, to facilitate conjugation to a lipid PK/PD modulator precursor.

Groups 2-10 comprise an αvβ6 integrin ligand Peptide 1 conjugated to the 5′ end of the sense strand according to procedures described in Example 5, above. Each of groups 2-10 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 32 below.

TABLE 32
Average relative MSTN expression from serum for mice of Example 9.

| | Day 8 | | Day 15 | | Day 22 | |
|---|---|---|---|---|---|---|
| Group | Avg | Std Dev | Avg | Std Dev | Avg | Std Dev |
| 1. Saline | 0.978 | 0.132 | 1.220 | 0.077 | 1.035 | 0.112 |
| 2. Avb6-Pep1-AD06569-LP38b | 0.462 | 0.016 | 0.378 | 0.048 | 0.278 | 0.011 |
| 3. Avb6-Pep1-AD06569-LP101b | 0.413 | 0.022 | 0.344 | 0.050 | 0.245 | 0.053 |
| 4. Avb6-Pep1-AD06569-LP41b | 0.375 | 0.052 | 0.316 | 0.041 | 0.231 | 0.013 |
| 5. Avb6-Pep1-AD06569-LP104b | 0.339 | 0.028 | 0.289 | 0.040 | 0.186 | 0.028 |
| 6. Avb6-Pep1-AD06569-LP53b | 0.422 | 0.016 | 0.374 | 0.022 | 0.256 | 0.045 |
| 7. Avb6-Pep1-AD06569-LP43b | 0.356 | 0.056 | 0.316 | 0.067 | 0.209 | 0.042 |
| 8. Avb6-Pep1-AD06569-LP44b | 0.369 | 0.014 | 0.315 | 0.037 | 0.233 | 0.014 |
| 9. Avb6-Pep1-AD06569-LP102b | 0.623 | 0.016 | 0.660 | 0.032 | 0.473 | 0.019 |
| 10. Avb6-Pep1-AD06569-LP103b | 0.382 | 0.022 | 0.340 | 0.062 | 0.211 | 0.032 |

Tissue collected from the gastrocnemius and triceps was used in a TaqMan assay to determine the relative amounts of MSTN in those tissues. Table 33, below, shows the results of the assay.

TABLE 33
Relative Expression in Triceps and Gastrocnemius in dosing groups of
Example 9.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.140 | 0.162 | 1.000 | 0.082 | 0.089 |
| 2. Avb6-Pep1-AD06569- | 0.306 | 0.093 | 0.134 | 0.215 | 0.059 | 0.082 |
| LP38b | | | | | | |
| 3. Avb6-Pep1-AD06569- | 0.288 | 0.043 | 0.050 | 0.130 | 0.020 | 0.023 |
| LP101b | | | | | | |
| 4. Avb6-Pep1-AD06569- | 0.238 | 0.042 | 0.051 | 0.140 | 0.037 | 0.051 |
| LP41b | | | | | | |
| 5. Avb6-Pep1-AD06569- | 0.187 | 0.034 | 0.042 | 0.126 | 0.017 | 0.019 |
| LP104b | | | | | | |
| 6. Avb6-Pep1-AD06569- | 0.249 | 0.042 | 0.051 | 0.123 | 0.028 | 0.036 |
| LP53b | | | | | | |
| 7. Avb6-Pep1-AD06569- | 0.196 | 0.040 | 0.051 | 0.111 | 0.036 | 0.054 |
| LP43b | | | | | | |
| 8. Avb6-Pep1-AD06569- | 0.238 | 0.037 | 0.044 | 0.144 | 0.009 | 0.010 |
| LP44b | | | | | | |
| 9. Avb6-Pep1-AD06569- | 0.526 | 0.076 | 0.089 | 0.326 | 0.024 | 0.026 |
| LP102b | | | | | | |
| 10. Avb6-Pep1-AD06569- | 0.239 | 0.050 | 0.064 | 0.133 | 0.012 | 0.013 |
| LP103b | | | | | | |

### Example 10. In Vivo Administration of RNAi Triggers Targeting Mstn in Mice

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 34
Dosing Groups for mice of Example 10.

| | Compound of Invention | |
|---|---|---|
| Group | Comprising RNAi Agent and Dose | Dosing Regimen |
| 1 | Saline | Single Injection on Day 1 |
| 2 | 2 mpk Avb6-Pep1-AD06569-LP38b | Single Injection on Day 1 |
| 6 | 2 mpk Avb6-Pep1-AD06569-LP54b | Single Injection on Day 1 |
| 7 | 2 mpk Avb6-Pep1-AD06569-LP93b | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the targeting ligand. The RNAi agent was also synthesized having a (C6-SS-C6) group on the 3′ end, to facilitate conjugation to a lipid PK/PD modulator precursor.

Groups 2 and 6-8 comprise an αvβ6 integrin ligand Peptide 1 conjugated to the 5′ end of the sense strand according to procedures described in Example 5, above. Each of groups 2 and 6-7 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 35 below.

TABLE 35
Average relative MSTN expression
from serum for mice of Example 10.

| | Day 8 | | Day 15 | | Day 22 | |
|---|---|---|---|---|---|---|
| Group | Avg | Std Dev | Avg | Std Dev | Avg | Std Dev |
| 1. Saline | 1.017 | 0.062 | 0.950 | 0.062 | 1.064 | 0.086 |
| 2. Avb6-Pep1- | 0.432 | 0.061 | 0.280 | 0.036 | 0.303 | 0.029 |
| AD06569- | | | | | | |
| LP38b | | | | | | |
| 6. Avb6-Pep1- | 0.468 | 0.053 | 0.274 | 0.034 | 0.266 | 0.011 |
| AD06569- | | | | | | |
| LP54b | | | | | | |
| 7. Avb6-Pep1- | 0.550 | 0.256 | 0.388 | 0.217 | 0.352 | 0.060 |
| AD06569- | | | | | | |
| LP93b | | | | | | |

Tissue collected from the gastrocnemius and triceps was used in a TaqMan assay to determine the relative amounts of MSTN in those tissues. Table 36, below, shows the results of the assay.

TABLE 36
Relative Expression in Triceps and Gastrocnemius in dosing groups of
Example 10.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.145 | 0.169 | 1.000 | 0.106 | 0.119 |
| 2. Avb6-Pep1-AD06569-LP38b | 0.296 | 0.038 | 0.043 | 0.186 | 0.017 | 0.019 |
| 6. Avb6-Pep1-AD06569-LP54b | 0.217 | 0.030 | 0.035 | 0.145 | 0.024 | 0.029 |
| 7. Avb6-Pep1-AD06569-LP93b | 0.291 | 0.040 | 0.046 | 0.196 | 0.040 | 0.050 |

### Example 11. In Vivo Administration of RNAi Triggers Targeting Mstn in Mice

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 37
Dosing Groups for mice of Example 11.

| | Compound of Invention | |
|---|---|---|
| Group | Comprising RNAi Agent and Dose | Dosing Regimen |
| 1 | Saline | Single Injection on Day 1 |
| 2 | 2 mpk Avb6-Pep1-AD06569-LP38b | Single Injection on Day 1 |
| 3 | 2 mpk Avb6-Pep1-AD06569-LP42b | Single Injection on Day 1 |
| 4 | 2 mpk Avb6-Pep1-AD06569-LP61b | Single Injection on Day 1 |
| 5 | 2 mpk Avb6-Pep1-AD06569-LP48b | Single Injection on Day 1 |
| 6 | 2 mpk Avb6-Pep1-AD06569-LP49b | Single Injection on Day 1 |
| 7 | 2 mpk Avb6-Pep1-AD06569-LP47b | Single Injection on Day 1 |
| 8 | 2 mpk Avb6-Pep1-AD06569-LP45b | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the targeting ligand. The RNAi agent was also synthesized having a (C6-SS-C6) group on the 3′ end, to facilitate conjugation to a lipid PK/PD modulator precursor.

Groups 2-8 comprise an αvβ6 integrin ligand Peptide 1 conjugated to the 5′ end of the sense strand according to procedures described in Example 5, above. Each of groups 2-8 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 38 below.

TABLE 38
Average relative MSTN expression
from serum for mice of Example 11.

| | Day 8 | | Day 15 | | Day 22 | |
|---|---|---|---|---|---|---|
| Group | Avg | Std Dev | Avg | Std Dev | Avg | Std Dev |
| 1. Saline | 0.949 | 0.058 | 0.916 | 0.086 | 1.118 | 0.117 |
| 2. Avb6-Pep1- | 0.441 | 0.050 | 0.320 | 0.017 | 0.289 | 0.036 |
| AD06569- | | | | | | |
| LP38b | | | | | | |
| 3. Avb6-Pep1- | 0.508 | 0.064 | 0.354 | 0.020 | 0.372 | 0.045 |
| AD06569- | | | | | | |
| LP42b | | | | | | |
| 4. Avb6-Pep1- | 0.299 | 0.060 | 0.220 | 0.030 | 0.203 | 0.031 |
| AD06569- | | | | | | |
| LP61b | | | | | | |
| 5. Avb6-Pep1- | 0.378 | 0.039 | 0.298 | 0.021 | 0.295 | 0.020 |
| AD06569- | | | | | | |
| LP48b | | | | | | |
| 6. Avb6-Pep1- | 0.344 | 0.038 | 0.244 | 0.014 | 0.236 | 0.021 |
| AD06569- | | | | | | |
| LP49b | | | | | | |
| 7. Avb6-Pep1- | 0.408 | 0.038 | 0.335 | 0.043 | 0.336 | 0.060 |
| AD06569- | | | | | | |
| LP47b | | | | | | |
| 8. Avb6-Pep1- | 0.357 | 0.047 | 0.280 | 0.016 | 0.244 | 0.021 |
| AD06569- | | | | | | |
| LP45b | | | | | | |

Tissue collected from the gastrocnemius and triceps was used in a TaqMan assay to determine the relative amounts of MSTN in those tissues. Table 39, below, shows the results of the assay.

TABLE 39
Relative Expression in Triceps and Gastrocnemius in dosing groups of
Example 11.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.093 | 0.103 | 1.000 | 0.200 | 0.250 |
| 2. Avb6-Pep1-AD06569-LP38b | 0.194 | 0.017 | 0.019 | 0.150 | 0.015 | 0.016 |
| 3. Avb6-Pep1-AD06569-LP42b | 0.216 | 0.016 | 0.018 | 0.189 | 0.022 | 0.024 |
| 4. Avb6-Pep1-AD06569-LP61b | 0.115 | 0.019 | 0.023 | 0.104 | 0.023 | 0.029 |
| 5. Avb6-Pep1-AD06569-LP48b | 0.166 | 0.024 | 0.028 | 0.125 | 0.008 | 0.009 |
| 6. Avb6-Pep1-AD06569-LP49b | 0.111 | 0.013 | 0.014 | 0.107 | 0.009 | 0.010 |
| 7. Avb6-Pep1-AD06569-LP47b | 0.171 | 0.028 | 0.033 | 0.148 | 0.023 | 0.027 |
| 8. Avb6-Pep1-AD06569-LP45b | 0.131 | 0.025 | 0.031 | 0.086 | 0.014 | 0.017 |

### Example 12. In Vivo Administration of RNAi Triggers Targeting Mstn in Mice

On Study Day 1, mice were injected with either isotonic saline (vehicle control) or 1.5 mg/kg (mpk) of a compound of the invention comprising an RNAi agent as described herein formulated in isotonic saline according to the following dosing Groups, wherein AD06569 has the structure shown in Table 24 above.

TABLE 40
Dosing Groups for mice of Example 12.

| | Compound of Invention | |
|---|---|---|
| Group | Comprising RNAi Agent and Dose | Dosing Regimen |
| 1 | Saline | Single Injection on Day 1 |
| 2 | 1.5 mpk Avb6-SM45-(L4)-AD06569-LP1b | Single Injection on Day 1 |
| 3 | 1.5 mpk Avb6-Pep1-AD06569-LP33b | Single Injection on Day 1 |
| 4 | 1.5 mpk Avb6-Pep1-AD06569-LP39b | Single Injection on Day 1 |
| 5 | 1.5 mpk Avb6-Pep1-AD06569-LP41b | Single Injection on Day 1 |
| 6 | 1.5 mpk Avb6-Pep1-AD06569-LP57b | Single Injection on Day 1 |
| 7 | 1.5 mpk Avb6-Pep1-AD06569-LP58b | Single Injection on Day 1 |
| 8 | 1.5 mpk Avb6-Pep1-AD06569-LP59b | Single Injection on Day 1 |
| 9 | 1.5 mpk Avb6-Pep1-AD06569-LP60b | Single Injection on Day 1 |
| 10 | 1.5 mpk Avb6-Pep1-AD06569-LP62b | Single Injection on Day 1 |
| 1 | Saline | Single Injection on Day 1 |
| 2 | 1.5 mpk Avb6-SM45-(L4)-AD06569-LP1b | Single Injection on Day 1 |
| 3 | 1.5 mpk Avb6-Pep1-AD06569-LP33b | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the targeting ligand. The RNAi agent was also synthesized having a (C6-SS-C6) group on the 3′ end, to facilitate conjugation to a lipid PK/PD modulator precursor.

Group 2 comprises an αvβ6 integrin ligand of Compound 45b, conjugated to the 5′ end of the sense strand using Linker 4 according to procedures described in Example 5, above. Groups 3-10 comprise an αvβ6 integrin ligand Peptide 1 conjugated to the 5′ end of the sense strand according to procedures described in Example 5, above. Each of groups 2-10 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 41 below.

TABLE 41
Average relative MSTN expression from serum for mice of Example 12.

| | Day 8 | | Day 15 | | Day 22 | |
|---|---|---|---|---|---|---|
| Group | Avg | Std Dev | Avg | Std Dev | Avg | Std Dev |
| 1. Saline | 1.177 | 0.088 | 1.174 | 0.178 | 1.023 | 0.065 |
| 2. mpk Avb6-SM45b-(L4)- | 0.263 | 0.009 | 0.198 | 0.027 | 0.141 | 0.026 |
| AD06569-LP1b | | | | | | |
| 3. mpk Avb6-Pep1-AD06569- | 0.443 | 0.056 | 0.287 | 0.048 | 0.269 | 0.056 |
| LP33b | | | | | | |
| 4. mpk Avb6-Pep1-AD06569- | 0.557 | 0.182 | 0.466 | 0.229 | 0.410 | 0.245 |
| LP39b | | | | | | |
| 5. mpk Avb6-Pep1-AD06569- | 0.523 | 0.072 | 0.396 | 0.073 | 0.343 | 0.075 |
| LP41b | | | | | | |
| 6. mpk Avb6-Pep1-AD06569- | 0.508 | 0.030 | 0.409 | 0.041 | 0.343 | 0.026 |
| LP57b | | | | | | |
| 7. mpk Avb6-Pep1-AD06569- | 0.452 | 0.043 | 0.313 | 0.030 | 0.288 | 0.013 |
| LP58b | | | | | | |
| 8. mpk Avb6-Pep1-AD06569- | 0.466 | 0.043 | 0.278 | 0.033 | 0.252 | 0.026 |
| LP59b | | | | | | |
| 9. mpk Avb6-Pep1-AD06569- | 0.535 | 0.018 | 0.319 | 0.018 | 0.292 | 0.029 |
| LP60b | | | | | | |
| 10. mpk Avb6-Pep1-AD06569- | 0.435 | 0.052 | 0.346 | 0.045 | 0.258 | 0.042 |
| LP62b | | | | | | |

Tissue collected from the gastrocnemius and triceps was used in a TaqMan assay to determine the relative amounts of MSTN in those tissues. Table 42, below, shows the results of the assay.

TABLE 42
Relative Expression in Triceps and Gastrocnemius in dosing groups of Example 12.

| | Triceps | | | Gastrocnemius | | |
|---|---|---|---|---|---|---|
| | | Low | High | | Low | High |
| Group | Rel. Exp. | Error | Error | Rel. Exp. | Error | Error |
| 1. Saline | 1.000 | 0.040 | 0.042 | 1.000 | 0.042 | 0.044 |
| 2. mpk Avb6-SM45b-(L4)-AD06569-LP1b | 0.160 | 0.014 | 0.015 | 0.082 | 0.007 | 0.007 |
| 3. mpk Avb6-Pep1-AD06569-LP33b | 0.259 | 0.055 | 0.070 | 0.129 | 0.034 | 0.045 |
| 4. mpk Avb6-Pep1-AD06569-LP39b | 0.539 | 0.149 | 0.206 | 0.311 | 0.081 | 0.109 |
| 5. mpk Avb6-Pep1-AD06569-LP41b | 0.425 | 0.030 | 0.032 | 0.235 | 0.043 | 0.053 |
| 6. mpk Avb6-Pep1-AD06569-LP57b | 0.442 | 0.039 | 0.043 | 0.241 | 0.021 | 0.023 |
| 7. mpk Avb6-Pep1-AD06569-LP58b | 0.244 | 0.045 | 0.055 | 0.160 | 0.033 | 0.041 |
| 8. mpk Avb6-Pep1-AD06569-LP59b | 0.259 | 0.040 | 0.047 | 0.138 | 0.022 | 0.026 |
| 9. mpk Avb6-Pep1-AD06569-LP60b | 0.334 | 0.046 | 0.054 | 0.179 | 0.022 | 0.025 |
| 10. mpk Avb6-Pep1-AD06569-LP62b | 0.297 | 0.050 | 0.060 | 0.193 | 0.050 | 0.068 |

### Example 13. In Vivo Administration of RNAi Triggers Targeting Mstn in Mice

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 43
Dosing Groups for mice of Example 13.

| | Compound of Invention | |
|---|---|---|
| Group | Comprising RNAi Agent and Dose | Dosing Regimen |
| 1 | Saline | Single Injection on Day 1 |
| 2 | 2 mpk Avb6-Pep1-AD06569-LP41b | Single Injection on Day 1 |
| 4 | 2 mpk Avb6-Pep1-AD06569-LP106b | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the targeting ligand. The RNAi agent was also synthesized having a (C6-SS-C6) group on the 3′ end, to facilitate conjugation to a lipid PK/PD modulator precursor.

Groups 2 and 4 comprise an αvβ6 integrin ligand Peptide 1 conjugated to the 5′ end of the sense strand according to procedures described in Example 5, above. Groups 2 and 4 comprise a lipid PK/PD modulator, with structures as supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 44 below.

TABLE 44
Average relative MSTN expression
from serum for mice of Example 13.

| | Day 8 | | Day 15 | | Day 22 | |
|---|---|---|---|---|---|---|
| Group | Avg | Std Dev | Avg | Std Dev | Avg | Std Dev |
| 1. Saline | 1.020 | 0.048 | 1.358 | 0.040 | 1.412 | 0.082 |
| 2. Avb6-Pep1- | 0.535 | 0.297 | 0.594 | 0.354 | 0.552 | 0.424 |
| AD06569- | | | | | | |
| LP41b | | | | | | |
| 4. Avb6-Pep1- | 0.413 | 0.022 | 0.419 | 0.038 | 0.341 | 0.027 |
| AD06569- | | | | | | |
| LP106b | | | | | | |

Tissue collected from the gastrocnemius and triceps was used in a TaqMan assay to determine the relative amounts of MSTN in those tissues. Table 45, below, shows the results of the assay.

TABLE 45
Relative Expression in Triceps and Gastrocnemius in dosing
groups of Example 13.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.180 | 0.219 | 1.000 | 0.109 | 0.123 |
| 2. Avb6-Pep1-AD06569- | 0.260 | 0.123 | 0.233 | 0.188 | 0.107 | 0.250 |
| LP41b | | | | | | |
| 4. Avb6-Pep1-AD06569- | 0.203 | 0.020 | 0.022 | 0.122 | 0.015 | 0.017 |
| LP106b | | | | | | |

### Example 14. In Vivo Administration of RNAi Triggers Targeting Mstn in Mice

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 46
Dosing Groups for mice of Example 14.

| | Compound of Invention | |
|---|---|---|
| Group | Comprising RNAi Agent and Dose | Dosing Regimen |
| 1 | Saline | Single Injection on Day 1 |
| 2 | 2 mpk Avb6-Pep1-AD06569-LP38b | Single Injection on Day 1 |
| 3 | 2 mpk Avb6-Pep1-AD07724-LP107b | Single Injection on Day 1 |
| 4 | 2 mpk Avb6-Pep1-AD07724-LP108b | Single Injection on Day 1 |
| 5 | 2 mpk Avb6-Pep1-AD07724-LP109b | Single Injection on Day 1 |
| 8 | 2 mpk Avb6-Pep1-AD06569-LP110b | Single Injection on Day 1 |
| 9 | 2 mpk Avb6-Pep1-AD06569-LP111b | Single Injection on Day 1 |

The RNAi agents AD06569 and AD07724 were synthesized having a nucleotide sequence targeted to the MSTN gene, and included a functionalized amine reactive group (NH2-C6)s at the 5′ terminal end of the sense strand to facilitate conjugation to the targeting ligand. AD06569 was also synthesized having a (C6-SS-C6) group on the 3′ end, to facilitate conjugation to a lipid PK/PD modulator precursor. AD07724 was synthesized having a terminal uAlk (see Table 22) residue, to facilitate conjugation to a lipid PK/PD modulator precursor.

Groups 2-9 comprise an αvβ6 integrin ligand Peptide 1 conjugated to the 5′ end of the sense strand according to procedures described in Example 5, above. Each of groups 2-9 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 47 below.

TABLE 47
Average relative MSTN expression from serum
for mice of Example 14.

| | Day 8 | | Day 15 | | Day 22 | |
|---|---|---|---|---|---|---|
| Group | Avg | Std Dev | Avg | Std Dev | Avg | Std Dev |
| 1. Saline | 1.071 | 0.061 | 0.988 | 0.086 | 1.170 | 0.088 |
| 2. Avb6-Pep1- | 0.500 | 0.017 | 0.254 | 0.026 | 0.315 | 0.026 |
| AD06569-LP38b | | | | | | |
| 3. Avb6-Pep1- | 0.804 | 0.026 | 0.595 | 0.061 | 0.753 | 0.008 |
| AD07724-LP107b | | | | | | |
| 4. Avb6-Pep1- | 0.877 | 0.052 | 0.626 | 0.041 | 0.732 | 0.053 |
| AD07724-LP108b | | | | | | |
| 5. Avb6-Pep1- | 0.859 | 0.100 | 0.610 | 0.059 | 0.709 | 0.079 |
| AD07724-LP109b | | | | | | |
| 8. Avb6-Pep1- | 0.476 | 0.045 | 0.306 | 0.023 | 0.317 | 0.040 |
| AD06569-LP110b | | | | | | |
| 9. Avb6-Pep1- | 0.422 | 0.021 | 0.220 | 0.028 | 0.250 | 0.039 |
| AD06569-LP111b | | | | | | |

Tissue collected from the gastrocnemius and triceps was used in a TaqMan assay to determine the relative amounts of MSTN in those tissues. Table 48, below, shows the results of the assay.

TABLE 48
Relative Expression in Triceps and Gastrocnemius in dosing
groups of Example 14.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.031 | 0.032 | 1.000 | 0.216 | 0.276 |
| 2. Avb6-Pep1-AD06569- | 0.193 | 0.028 | 0.032 | 0.109 | 0.008 | 0.008 |
| LP38b | | | | | | |
| 3. Avb6-Pep1-AD07724- | 0.506 | 0.045 | 0.050 | 0.347 | 0.047 | 0.055 |
| LP107b | | | | | | |
| 4. Avb6-Pep1-AD07724- | 0.441 | 0.046 | 0.052 | 0.269 | 0.049 | 0.060 |
| LP108b | | | | | | |
| 5. Avb6-Pep1-AD07724- | 0.447 | 0.057 | 0.065 | 0.284 | 0.066 | 0.086 |
| LP109b | | | | | | |
| 8. Avb6-Pep1-AD06569- | 0.193 | 0.023 | 0.026 | 0.105 | 0.015 | 0.017 |
| LP110b | | | | | | |
| 9. Avb6-Pep1-AD06569- | 0.129 | 0.024 | 0.029 | 0.094 | 0.016 | 0.019 |
| LP111b | | | | | | |

### Example 15. In Vivo Administration of RNAi Triggers Targeting Mstn in Mice

On Study Day 1, mice were injected with either isotonic saline (vehicle control) or 2 mg/kg (mpk) of a compound of the invention comprising an RNAi agent as described herein formulated in isotonic saline according to the following dosing Groups, wherein AD06569 has the structure shown in Table 24 above.

TABLE 49
Dosing Groups for mice of Example 15.

| | Compound of Invention | |
|---|---|---|
| Group | Comprising RNAi Agent and Dose | Dosing Regimen |
| 1 | Saline | Single Injection on Day 1 |
| 2 | 2 mpk Avb6-Pep1-AD06569-LP38b | Single Injection on Day 1 |
| 3 | 2 mpk Avb6-Pep1-AD07724-LP108b | Single Injection on Day 1 |
| 4 | 2 mpk Avb6-Pep1-AD07909-LP108b | Single Injection on Day 1 |
| 5 | 2 mpk Avb6-Pep1-AD07910-LP108b | Single Injection on Day 1 |
| 6 | 2 mpk Avb6-Pep1-AD06569-LP143b | Single Injection on Day 1 |
| 7 | 2 mpk Avb6-Pep6-AD06569-LP143b | Single Injection on Day 1 |
| 8 | 2 mpk Avb6-Pep1-AD06569-LP57b | Single Injection on Day 1 |
| 9 | 2 mpk Avb6-Pep6-AD06569-LP130b | Single Injection on Day 1 |
| 10 | 2 mpk Avb6-Pep1-AD06569-LP124b | Single Injection on Day 1 |

The RNAi agents AD06569, AD07724, AD07909 and AD07910 were synthesized having a nucleotide sequence targeted to the MSTN gene, and included a functionalized amine reactive group (NH2-C6)s at the 5′ terminal end of the sense strand to facilitate conjugation to the targeting ligand. AD06569 was also synthesized having a (C6-SS-C6) group on the 3′ end, to facilitate conjugation to a lipid PK/PD modulator precursor. AD07724, AD07909, and AD07910 were synthesized having a terminal alkyne-containing nucleotide (see Table 22), to facilitate conjugation to a lipid PK/PD modulator precursor.

Groups 2-6, 8 and 10 comprise an αvβ6 integrin ligand Peptide 1 conjugated to the 5′ end of the sense strand according to procedures described in Example 5, above. Groups 7 and 9 comprise an αvβ6 integrin ligand Peptide 6 conjugated to the 5′ end of the sense strand according to procedures described in Example 5, above. Each of groups 2-10 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 50 below.

TABLE 49
Average relative MSTN expression from serum for mice of Example 15.

| | Day 8 | | Day 15 | | Day 22 | |
|---|---|---|---|---|---|---|
| Group | Avg | Std Dev | Avg | Std Dev | Avg | Std Dev |
| 1. Saline | 1.087 | 0.059 | 1.011 | 0.075 | 1.036 | 0.095 |
| 2. Avb6-Pep1- | 0.453 | 0.020 | 0.317 | 0.030 | 0.272 | 0.018 |
| AD06569-LP38b | | | | | | |
| 3. Avb6-Pep1- | 0.833 | 0.029 | 0.574 | 0.056 | 0.586 | 0.057 |
| AD07724-LP108b | | | | | | |
| 4. Avb6-Pep1- | 0.792 | 0.108 | 0.587 | 0.062 | 0.543 | 0.077 |
| AD07909-LP108b | | | | | | |
| 5. Avb6-Pep1- | 0.585 | 0.022 | 0.347 | 0.035 | 0.347 | 0.070 |
| AD07910-LP108b | | | | | | |
| 6. Avb6-Pep1- | 0.495 | 0.060 | 0.331 | 0.043 | 0.283 | 0.054 |
| AD06569-LP143b | | | | | | |
| 7. Avb6-Pep6- | 0.406 | 0.003 | 0.286 | 0.024 | 0.255 | 0.048 |
| AD06569-LP143b | | | | | | |
| 8. Avb6-Pep1- | 0.437 | 0.064 | 0.265 | 0.037 | 0.241 | 0.022 |
| AD06569-LP57b | | | | | | |
| 9. Avb6-Pep6- | 0.443 | 0.032 | 0.269 | 0.010 | 0.212 | 0.008 |
| AD06569-LP130b | | | | | | |
| 10. Avb6-Pep1- | 0.406 | 0.040 | 0.257 | 0.027 | 0.212 | 0.026 |
| AD06569-LP124b | | | | | | |

Tissue collected from the gastrocnemius and triceps was used in a TaqMan assay to determine the relative amounts of MSTN in those tissues. Table 51, below, shows the results of the assay.

TABLE 51
Relative Expression in Triceps and Gastrocnemius in
dosing groups of Example 15.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.124 | 0.141 | 1.000 | 0.109 | 0.122 |
| 2. Avb6-Pep1-AD06569- | 0.263 | 0.035 | 0.040 | 0.175 | 0.014 | 0.015 |
| LP38b | | | | | | |
| 3. Avb6-Pep1-AD07724- | 0.435 | 0.021 | 0.022 | 0.317 | 0.023 | 0.025 |
| LP108b | | | | | | |
| 4. Avb6-Pep1-AD07909- | 0.477 | 0.044 | 0.049 | 0.328 | 0.037 | 0.041 |
| LP108b | | | | | | |
| 5. Avb6-Pep1-AD07910- | 0.229 | 0.029 | 0.033 | 0.130 | 0.037 | 0.051 |
| LP108b | | | | | | |
| 6. Avb6-Pep1-AD06569- | 0.228 | 0.045 | 0.056 | 0.170 | 0.032 | 0.039 |
| LP143b | | | | | | |
| 7. Avb6-Pep6-AD06569- | 0.194 | 0.042 | 0.053 | 0.135 | 0.031 | 0.041 |
| LP143b | | | | | | |
| 8. Avb6-Pep1-AD06569- | 0.156 | 0.012 | 0.013 | 0.084 | 0.019 | 0.024 |
| LP57b | | | | | | |
| 9. Avb6-Pep6-AD06569- | 0.183 | 0.046 | 0.061 | 0.112 | 0.035 | 0.052 |
| LP130b | | | | | | |
| 10. Avb6-Pep1-AD06569- | 0.166 | 0.031 | 0.039 | 0.100 | 0.019 | 0.023 |
| LP124b | | | | | | |

### Example 16. In Vivo Administration of RNAi Triggers Targeting Mstn in Mice

On Study Day 1, mice were injected with either isotonic saline (vehicle control) or 1 mg/kg (mpk) of a compound of the invention comprising an RNAi agent as described herein formulated in isotonic saline according to the following dosing Groups set forth in Table 52, wherein AD06569 has the structure shown in Table 24 above.

TABLE 52
Dosing Groups for Mice of Example 16.

| | Compound of Invention | |
|---|---|---|
| Group | Comprising RNAi Agent and Dose | Dosing Regimen |
| 1 | Saline | Single Injection on Day 1 |
| 2 | 1 mpk Avb6-Pep1- | Single Injection on Day 1 |
| | AD06569-LP29b | |
| 3 | 1 mpk Avb6-Pep1- | Single Injection on Day 1 |
| | AD06569-LP217b | |
| 4 | 1 mpk Avb6-Pep1- | Single Injection on Day 1 |
| | AD06569-LP220b | |
| 5 | 1 mpk Avb6-Pep1- | Single Injection on Day 1 |
| | AD06569-LP221b | |
| 6 | 1 mpk Avb6-Pep1- | Single Injection on Day 1 |
| | AD06569-LP223b | |
| 7 | 1 mpk Avb6-Pep1- | Single Injection on Day 1 |
| | AD06569-LP224b | |
| 8 | 1 mpk Avb6-Pep1- | Single Injection on Day 1 |
| | AD06569-LP225b | |
| 9 | 1 mpk Avb6-Pep1- | Single Injection on Day 1 |
| | AD08257-LP226b | |

The RNAi agents AD06569 and AD08257 were synthesized having a nucleotide sequence targeted to the MSTN gene. AD0659 included a functionalized amine reactive group (NH2-C6)s at the 5′ terminal end of the sense strand to facilitate conjugation to the targeting ligand. AD06569 was also synthesized having a (C6-SS-C6) group on the 3′ end, to facilitate conjugation to a lipid PK/PD modulator precursor. AD08257 included a (NH2-C6)s group at the 5′ terminal end of the sense strand to facilitate conjugation to the targeting ligand. AD08257 was also synthesized having an L_{A2} group on the 3′ end, to facilitate conjugation to a lipid PK/PD modulator precursor.

Groups 2-9 comprise an αvβ6 integrin ligand Peptide 1 conjugated to the 5′ end of the sense strand according to procedures described in Example 5, above. Each of groups 2-9 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 53 below.

TABLE 53
Average relative MSTN expression from serum for mice of Example 16.

| | Day 8 | | Day 15 | | Day 22 | |
|---|---|---|---|---|---|---|
| Group | Avg | Std Dev | Avg | Std Dev | Avg | Std Dev |
| 1. Saline | 0.902 | 0.078 | 0.989 | 0.071 | 0.969 | 0.100 |
| 2. Avb6-Pep1- | 0.468 | 0.070 | 0.314 | 0.032 | 0.265 | 0.016 |
| AD06569-LP29b | | | | | | |
| 3. Avb6-Pep1- | 0.457 | 0.029 | 0.366 | 0.054 | 0.298 | 0.027 |
| AD06569-LP217b | | | | | | |
| 4. Avb6-Pep1- | 0.453 | 0.018 | 0.330 | 0.011 | 0.277 | 0.035 |
| AD06569-LP220b | | | | | | |
| 5. Avb6-Pep1- | 0.619 | 0.039 | 0.567 | 0.072 | 0.443 | 0.067 |
| AD06569-LP221b | | | | | | |
| 6. Avb6-Pep1- | 0.490 | 0.027 | 0.353 | 0.022 | 0.273 | 0.017 |
| AD06569-LP223b | | | | | | |
| 7. Avb6-Pep1- | 0.467 | 0.074 | — | — | 0.298 | 0.021 |
| AD06569-LP224b | | | | | | |
| 8. Avb6-Pep1- | 0.445 | 0.023 | — | — | 0.233 | 0.070 |
| AD06569-LP225b | | | | | | |
| 9. Avb6-Pep1- | 0.527 | 0.042 | 0.444 | 0.067 | 0.356 | 0.074 |
| AD08257-LP226b | | | | | | |

Tissue collected from the triceps was used in a TaqMan assay to determine the relative amounts of MSTN in those tissues. Table 54, below, shows the results of the assay.

TABLE 54
Relative Expression in Triceps in dosing groups of Example 16.

| | Triceps | | |
|---|---|---|---|
| | Rei. | Low | High |
| Group | Exp. | Error | Error |
| 1. Saline | 1.000 | 0.047 | 0.050 |
| 2. Avb6-Pep1-AD06569-LP29b | 0.239 | 0.038 | 0.045 |
| 3. Avb6-Pep1-AD06569-LP217b | 0.310 | 0.041 | 0.047 |
| 4. Avb6-Pep1-AD06569-LP220b | 0.264 | 0.022 | 0.024 |
| 5. Avb6-Pep1-AD06569-LP221b | 0.410 | 0.070 | 0.084 |
| 6. Avb6-Pep1-AD06569-LP223b | 0.265 | 0.037 | 0.043 |
| 7. Avb6-Pep1-AD06569-LP224b | 0.314 | 0.052 | 0.062 |
| 8. Avb6-Pep1-AD06569-LP225b | 0.281 | 0.044 | 0.052 |
| 9. Avb6-Pep1-AD08257-LP226b | 0.243 | 0.044 | 0.054 |

### Example 17. In Vivo Administration of RNAi Triggers Targeting Mstn in Mice

On Study Day 1, mice were injected with isotonic saline (vehicle control), 0.75 mg/kg (mpk) of a compound of the invention comprising an RNAi agent as described herein formulated in isotonic saline, or 2 mpk of a compound of the invention comprising an RNAi agent as described herein formulated in isotonic saline according to the dosing Groups set forth in Table 55, wherein AD06569 has the structure shown in Table 24 above.

TABLE 55
Dosing Groups for Mice of Example 17.

| | Compound of Invention | |
|---|---|---|
| Group | Comprising RNAi Agent and Dose | Dosing Regimen |
| 1 | Saline | Single Injection on Day 1 |
| 2 | 0.75 mpk Avb6-Pep1- | Single Injection on Day 1 |
| | AD06569-LP29b | |
| 3 | 0.75 mpk Avb6-Pep1- | Single Injection on Day 1 |
| | AD06569-LP210b | |
| 4 | 0.75 mpk Avb6-Pep1- | Single Injection on Day 1 |
| | AD06569-LP220b | |
| 5 | 0.75 mpk Avb6-Pep1- | Single Injection on Day 1 |
| | AD06569-LP238b | |
| 6 | 2 mpk Avb6-Pep1- | Single Injection on Day 1 |
| | AD06569-LP29b | |
| 7 | 2 mpk Avb6-Pep1- | Single Injection on Day 1 |
| | AD06569-LP210b | |
| 8 | 2 mpk Avb6-Pep1- | Single Injection on Day 1 |
| | AD06569-LP220b | |
| 9 | 2 mpk Avb6-Pep1- | Single Injection on Day 1 |
| | AD06569-LP238b | |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the targeting ligand. AD06569 was also synthesized having a (C6-SS-C6) group on the 3′ end, to facilitate conjugation to a lipid PK/PD modulator precursor.

Groups 2-9 comprise an αvβ6 integrin ligand Peptide 1 conjugated to the 5′ end of the sense strand according to procedures described in Example 5, above. Each of groups 2-9 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 56 below.

TABLE 56
Average relative MSTN expression from serum for mice of Example 17.

| | Day 8 | | Day 15 | | Day 22 | |
|---|---|---|---|---|---|---|
| | | Std | | Std | | Std |
| Group | Avg | Dev | Avg | Dev | Avg | Dev |
| 1. Saline | 0.966 | 0.094 | 0.903 | 0.033 | 1.065 | 0.089 |
| 2. 0.75 mpk Avb6- | 0.518 | 0.048 | 0.398 | 0.049 | 0.391 | 0.039 |
| Pep1-AD06569-LP29b | | | | | | |
| 3. 0.75 mpk Avb6- | 0.478 | 0.093 | 0.421 | 0.023 | 0.444 | 0.008 |
| Pep1-AD06569-LP210b | | | | | | |
| 4. 0.75 mpk Avb6- | 0.447 | 0.050 | 0.343 | 0.059 | 0.323 | 0.055 |
| Pep1-AD06569-LP220b | | | | | | |
| 5. 0.75 mpk Avb6- | 0.510 | 0.034 | 0.373 | 0.029 | 0.412 | 0.044 |
| Pep1-AD06569-LP238b | | | | | | |
| 6. 2 mpk Avb6-Pep1- | 0.479 | 0.028 | 0.329 | 0.031 | 0.310 | 0.023 |
| AD06569-LP29b | | | | | | |
| 7. 2 mpk Avb6-Pep1- | 0.444 | 0.026 | 0.305 | 0.049 | 0.295 | 0.033 |
| AD06569-LP210b | | | | | | |
| 8. 2 mpk Avb6-Pep1- | 0.453 | 0.055 | 0.296 | 0.023 | 0.285 | 0.028 |
| AD06569-LP220b | | | | | | |
| 9. 2 mpk Avb6-Pep1- | 0.410 | 0.073 | 0.304 | 0.027 | 0.290 | 0.021 |
| AD06569-LP238b | | | | | | |

Tissue collected from the gastrocnemius and triceps was used in a TaqMan assay to determine the relative amounts of MSTN in those tissues. Table 57, below, shows the results of the assay.

TABLE 57
Relative Expression in Triceps and Gastrocnemius in
dosing groups of Example 17.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.034 | 0.036 | 1.000 | 0.034 | 0.035 |
| 2. 0.75 mpk Avb6-Pep1- | 0.350 | 0.091 | 0.122 | 0.251 | 0.070 | 0.096 |
| AD06569-LP29b | | | | | | |
| 3. 0.75 mpk Avb6-Pep1- | 0.278 | 0.049 | 0.059 | 0.199 | 0.027 | 0.031 |
| AD06569-LP210b | | | | | | |
| 4. 0.75 mpk Avb6-Pep1- | 0.211 | 0.023 | 0.026 | 0.155 | 0.017 | 0.019 |
| AD06569-LP220b | | | | | | |
| 5. 0.75 mpk Avb6-Pep1- | 0.304 | 0.024 | 0.027 | 0.214 | 0.015 | 0.017 |
| AD06569-LP238b | | | | | | |
| 6. 2 mpk Avb6-Pep1- | 0.170 | 0.046 | 0.063 | 0.119 | 0.023 | 0.028 |
| AD06569-LP29b | | | | | | |
| 7. 2 mpk Avb6-Pep1- | 0.223 | 0.055 | 0.073 | 0.149 | 0.041 | 0.056 |
| AD06569-LP210b | | | | | | |
| 8. 2 mpk Avb6-Pep1- | 0.208 | 0.023 | 0.026 | 0.136 | 0.023 | 0.028 |
| AD06569-LP220b | | | | | | |
| 9. 2 mpk Avb6-Pep1- | 0.225 | 0.029 | 0.033 | 0.138 | 0.027 | 0.033 |
| AD06569-LP238b | | | | | | |

### Example 18. In Vivo Administration of RNAi Triggers Targeting Mstn in Mice

On Study Day 1, mice were injected with isotonic saline (vehicle control), 0.75 mg/kg (mpk) of a compound of the invention comprising an RNAi agent as described herein formulated in isotonic saline, or 2 mpk of a compound of the invention comprising an RNAi agent as described herein formulated in isotonic saline according to the dosing Groups set forth in Table 58, wherein AD06569 has the structure shown in Table 24 above.

TABLE 58
Dosing Groups for Mice of Example 18.

| | Compound of Invention | |
|---|---|---|
| Group | Comprising RNAi Agent and Dose | Dosing Regimen |
| 1 | Saline | Single Injection on Day 1 |
| 2 | 0.75 mpk Avb6-Pep1-AD06569-LP29b | Single Injection on Day 1 |
| 3 | 0.75 mpk Avb6-Pep1-AD08257-LP240b | Single Injection on Day 1 |
| 4 | 0.75 mpk Avb6-Pep1-AD08257-LP246b | Single Injection on Day 1 |
| 5 | 0.75 mpk Avb6-Pep1-AD06569-LP247b | Single Injection on Day 1 |
| 6 | 2 mpk Avb6-Pep1-AD06569-LP29b | Single Injection on Day 1 |
| 7 | 2 mpk Avb6-Pep1-AD08257-LP240b | Single Injection on Day 1 |
| 8 | 2 mpk Avb6-Pep1-AD08257-LP246b | Single Injection on Day 1 |
| 9 | 2 mpk Avb6-Pep1-AD06569-LP247b | Single Injection on Day 1 |

The RNAi agents AD06569 and AD08257 were synthesized having a nucleotide sequence targeted to the MSTN gene. AD0659 included a functionalized amine reactive group (NH2-C6)s at the 5′ terminal end of the sense strand to facilitate conjugation to the targeting ligand. AD06569 was also synthesized having a (C6-SS-C6) group on the 3′ end, to facilitate conjugation to a lipid PK/PD modulator precursor. AD08257 included a (NH2-C6)s group at the 5′ terminal end of the sense strand to facilitate conjugation to the targeting ligand. AD08257 was also synthesized having an L_{A2} group on the 3′ end, to facilitate conjugation to a lipid PK/PD modulator precursor.

Groups 2-9 comprise an αvβ6 integrin ligand Peptide 1 conjugated to the 5′ end of the sense strand according to procedures described in Example 5, above. Each of groups 2-9 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 59 below.

TABLE 59
Average relative MSTN expression from serum for mice of Example 18.

| | Day 8 | | Day 15 | | Day 22 | |
|---|---|---|---|---|---|---|
| | | Std | | Std | | Std |
| Group | Avg | Dev | Avg | Dev | Avg | Dev |
| 1. Saline | 1.112 | 0.078 | 1.193 | 0.044 | 1.204 | 0.084 |
| 2. 0.75 mpk Avb6- | 0.642 | 0.078 | 0.440 | 0.039 | 0.408 | 0.042 |
| Pep1-AD06569-LP29b | | | | | | |
| 3. 0.75 mpk Avb6- | 0.662 | 0.121 | 0.488 | 0.028 | 0.444 | 0.070 |
| Pep1-AD08257-LP240b | | | | | | |
| 4. 0.75 mpk Avb6- | 0.614 | 0.082 | 0.479 | 0.078 | 0.418 | 0.051 |
| Pep1-AD08257-LP246b | | | | | | |
| 5. 0.75 mpk Avb6- | 0.612 | 0.051 | 0.460 | 0.060 | 0.447 | 0.019 |
| Pep1-AD06569-LP247b | | | | | | |
| 6. 2 mpk Avb6-Pep1- | 0.449 | 0.033 | 0.332 | 0.034 | 0.285 | 0.012 |
| AD06569-LP29b | | | | | | |
| 7. 2 mpk Avb6-Pep1- | 0.482 | 0.061 | 0.398 | 0.037 | 0.355 | 0.050 |
| AD08257-LP240b | | | | | | |
| 8. 2 mpk Avb6-Pep1- | 0.558 | 0.038 | 0.427 | 0.041 | 0.382 | 0.019 |
| AD08257-LP246b | | | | | | |
| 9. 2 mpk Avb6-Pep1- | 0.555 | 0.071 | 0.407 | 0.027 | 0.370 | 0.033 |
| AD06569-LP247b | | | | | | |

Tissue collected from the gastrocnemius and triceps was used in a TaqMan assay to determine the relative amounts of MSTN in those tissues. Table 60, below, shows the results of the assay.

TABLE 60
Relative Expression in Triceps and Gastrocnemius in
dosing groups of Example 18.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.102 | 0.114 | 1.000 | 0.118 | 0.134 |
| 2. 0.75 mpk Avb6-Pep1- | 0.272 | 0.055 | 0.069 | 0.220 | 0.043 | 0.053 |
| AD06569-LP29b | | | | | | |
| 3. 0.75 mpk Avb6-Pep1- | 0.358 | 0.055 | 0.065 | 0.256 | 0.041 | 0.049 |
| AD08257-LP240b | | | | | | |
| 4. 0.75 mpk Avb6-Pep1- | 0.280 | 0.080 | 0.113 | 0.206 | 0.063 | 0.091 |
| AD08257-LP246b | | | | | | |
| 5. 0.75 mpk Avb6-Pep1- | 0.271 | 0.032 | 0.037 | 0.189 | 0.023 | 0.027 |
| AD06569-LP247b | | | | | | |
| 6. 2 mpk Avb6-Pep1- | 0.196 | 0.014 | 0.015 | 0.135 | 0.019 | 0.022 |
| AD06569-LP29b | | | | | | |
| 7. 2 mpk Avb6-Pep1- | 0.210 | 0.033 | 0.040 | 0.135 | 0.034 | 0.046 |
| AD08257-LP240b | | | | | | |
| 8. 2 mpk Avb6-Pep1- | 0.228 | 0.034 | 0.041 | 0.163 | 0.045 | 0.062 |
| AD08257-LP246b | | | | | | |
| 9. 2 mpk Avb6-Pep1- | 0.183 | 0.024 | 0.028 | 0.138 | 0.035 | 0.047 |
| AD06569-LP247b | | | | | | |

### Example 19. In Vivo Administration of RNAi Triggers Targeting Mstn in Mice

On Study Day 1, mice were injected with either isotonic saline (vehicle control), 2 mg/kg (mpk) of a compound of the invention comprising an RNAi agent as described herein formulated in isotonic saline, or 2 mpk of a control compound according to the following dosing Groups, wherein AD06569 has the structure shown in Table 24 above.

TABLE 61
Dosing Groups for mice of Example 19.

| | Compound of Invention | |
|---|---|---|
| Group | Comprising RNAi Agent and Dose | Dosing Regimen |
| 1 | Saline | Single Injection on Day 1 |
| 2 | 2 mpk Avb6-Pep1-AD06569-LP29b | Single Injection on Day 1 |
| 3 | 2 mpk Avb6-Pep1-AD06569-nEm | Single Injection on Day 1 |
| 4 | 2 mpk AD06569 | Single Injection on Day 1 |
| 5 | 2 mpk Avb6-Pep1-AD06569-bis-C16 | Single Injection on Day 1 |
| 6 | 2 mpk Avb6-Pep1-AD06569-bis-PEG47 | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the targeting ligand. AD06569 was also synthesized having a (C6-SS-C6) group on the 3′ end, to facilitate conjugation to a lipid PK/PD modulator precursor.

Groups 2, 3, 5 and 6 comprised an αvβ6 integrin ligand Peptide 1 conjugated to the 5′ end of the sense strand according to procedures described in Example 5, above. Group 2 comprised a PK/PD modulator, with structure as shown supra, conjugated to the 3′ end of the sense strand according to procedures described in Example 6, above. Group 3 included a capped maleimide conjugated to the 3′ end of the sense strand according to procedures described in Example 6, above. Group 4 included an RNAi agent with no targeting ligand or PK/PD modulator. Group 5 included a PK/PD modulator with bis-C16 with no PEG moiety adjacent to the lipid. The 3′ end of the sense strand of the RNAi agent of Group 5 was conjugated to a maleimide-containing PK/PD modulator precursor having the structure:

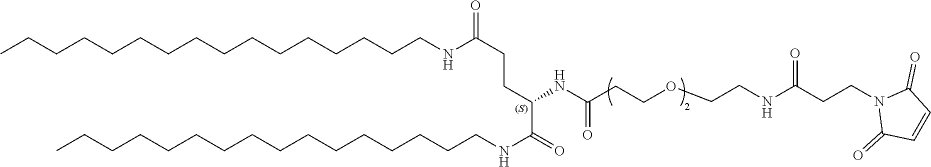

according to procedures described in Example 6, above. Group 6 included a PK/PD modulator with no lipid portion, and a bis-PEG47 moiety. The 3′ end of the sense strand of the RNAi agent of Group 6 was conjugated to a maleimide-containing PK/PD modulator precursor having the structure:

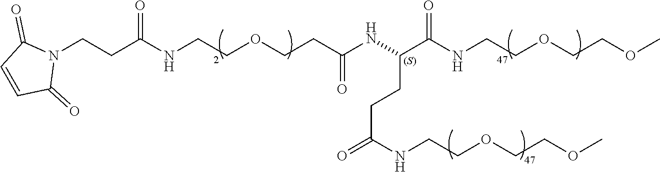

according to procedures described in Example 6, above.

Four (4) mice were dosed in each Group (n=4). Mice were bled and serum was collected on days 1, 8, 15 and 22. Mice were sacrificed on study day 22. Relative MSTN expression was determined by ELISA assay on mouse myostatin in serum. Average relative myostatin expression in serum is shown in Table 62 below.

TABLE 62
Average relative MSTN expression from serum for mice of Example 19.

| | Day 8 | | Day 15 | | Day 22 | |
|---|---|---|---|---|---|---|
| Group | Avg | Std Dev | Avg | Std Dev | Avg | Std Dev |
| 1. Saline | 1.091 | 0.177 | 1.200 | 0.052 | 1.001 | 0.075 |
| 2. Avb6-Pep1- | 0.492 | 0.073 | 0.353 | 0.055 | 0.289 | 0.026 |
| AD06569-LP29b | | | | | | |
| 3. Avb6-Pep1- | 0.857 | 0.182 | 0.634 | 0.123 | 0.587 | 0.087 |
| AD06569-nEm | | | | | | |
| 4. AD06569 | 1.361 | 0.226 | 1.276 | 0.039 | 1.211 | 0.197 |
| 5. Avb6-Pep1- | 0.634 | 0.060 | 0.470 | 0.091 | 0.379 | 0.072 |
| AD06569-bis-C16 | | | | | | |
| 6. Avb6-Pep1- | 0.752 | 0.059 | 0.585 | 0.094 | 0.516 | 0.082 |
| AD06569-bis- | | | | | | |
| PEG47 | | | | | | |

As shown in Table 62, the bis-PEG moiety adjacent to the lipid moiety (i.e., LP 29b) of Group 2 shows improved MSTN knockdown over the capped maleimide of Group 3, the “naked” RNAi agent of Group 4, the PK/PD modulator without PEG of Group 5, and the PK/PD modulator without lipid of Group 6.

### Example 20. In Vivo Administration of RNAi Triggers Targeting MSTN in Cynomolgus Monkeys

Myostatin RNAi agents that included a sense strand and an antisense strand were synthesized according to phosphoramidite technology on solid phase in accordance with general procedures known in the art and commonly used in oligonucleotide synthesis, as set forth in Example 1 herein. On Study Days 1, 7, and 28, cynomolgus macaque (Macaca fascicularis) primates (referred to herein as “cynos”) were injected with 10 mg/kg (mpk) of a compound of the invention comprising an RNAi agent as described herein formulated in isotonic saline according to the following dosing Groups:

TABLE 63
Dosing Groups for cynos of Example 20.

| | Compound of Invention | |
|---|---|---|
| Group | Comprising RNAi Agent and Dose | Dosing Regimen |
| 1 | 10 mpk αvβ6 peptide 1-Mstn(AD06569)-LP29b | Injections on |
| | | Days 1, 7, and 28 |

The RNAi agent in Example 20 was synthesized having nucleotide sequences directed to target the MSTN gene, and included a functionalized amine reactive group (NH2-C6)s at the 5′ terminal end of the sense strand to facilitate conjugation to the targeting ligand αvβ6 peptide 1. The RNAi agent further included a disulfide functional group (C6-SS-C6) at the 3′ terminal end of the sense strand to facilitate conjugation to a PK/PD modulator of structure LP 29b, shown supra.

Two (2) cynos were dosed in each Group (n=2). Serum samples were taken on days −28, −21, −14, −7, and day 1 (pre-dose). Monkeys were then administered according to the respective Groups as set forth in Table 22. Serum was then collected on day 8, day 15, day 22, day 29, day 36, day 43, day 50, day 57, day 64, day 71, day 78, day 85, day 99, day 113, and day 134. An ELISA assay was performed on serum samples to determine the amount of cyno myostatin in serum. Average myostatin in serum samples for Group 1 is shown in Table 64 below.

TABLE 64
Average cyno myostatin protein in serum in Group 1 of Example 20,
normalized to Day 1.

| Day −28 | | Day −21 | | Day −14 | | Day −7 | | Day 1 | |
|---|---|---|---|---|---|---|---|---|---|
| | Std | | Std | | Std | | Std | | Std |
| Avg | Dev | Avg | Dev | Avg | Dev | Avg | Dev | Avg | Dev |
| 1.160 | 0.041 | 1.135 | 0.064 | 1.045 | 0.051 | 1.085 | 0.056 | 1.000 | 0.000 |
| Day 8 | | Day 15 | | Day 22 | | Day 29 | | Day 36 | |
| | Std | | Std | | Std | | Std | | Std |
| Avg | Dev | Avg | Dev | Avg | Dev | Avg | Dev | Avg | Dev |
| 0.891 | 0.006 | 0.620 | 0.192 | 0.385 | 0.126 | 0.321 | 0.074 | 0.281 | 0.083 |
| Day 43 | | Day 50 | | Day 57 | | Day 64 | | Day 71 | |
| | Std | | Std | | Std | | Std | | Std |
| Avg | Dev | Avg | Dev | Avg | Dev | Avg | Dev | Avg | Dev |
| 0.231 | 0.058 | 0.250 | 0.033 | 0.213 | 0.020 | 0.282 | 0.042 | 0.228 | 0.018 |
| Day 78 | | Day 85 | | Day 99 | | Day 113 | | Day 134 | |
| | Std | | Std | | Std | | Std | | Std |
| Avg | Dev | Avg | Dev | Avg | Dev | Avg | Dev | Avg | Dev |
| 0.256 | 0.038 | 0.255 | 0.045 | 0.356 | 0.053 | 0.322 | 0.037 | 0.542 | 0.072 |

As shown in Table 64, robust and long-lasting knockdown of target genes can be achieved using compounds described herein.

### Example 21. In Vivo Administration of RNAi Triggers Targeting MSTN in Cynomolgus Monkeys

Myostatin RNAi agents that included a sense strand and an antisense strand were synthesized according to phosphoramidite technology on solid phase in accordance with general procedures known in the art and commonly used in oligonucleotide synthesis, as set forth in Example 1 herein. On Study Day 1, cynomolgus macaque (Macaca fascicularis) primates (referred to herein as “cynos”) were injected with 5 mg/kg, 10 mg/kg (mpk) or 20 mg/kg (mpk) of a compound of the invention comprising an RNAi agent as described herein formulated in isotonic saline according to the following dosing Groups:

TABLE 65
Dosing Groups for cynos of Example 21.

| | Compound of Invention | |
|---|---|---|
| Group | Comprising RNAi Agent and Dose | Dosing Regimen |
| 1 | 5 mpk αvβ6 peptide 1-Mstn(AD06569)-LP29b | Single Injection |
| | | on Day 1 |
| 2 | 10 mpk aαvβ6 peptide 1-Mstn(AD06569)-LP29b | Single Injection |
| | | on Day 1 |
| 3 | 20 mpk αvβ6 peptide 1-Mstn(AD06569)-LP29b | Single Injection |
| | | on Day 1 |

The RNAi agents in Example 21 were synthesized having nucleotide sequences directed to target the MSTN gene, and included a functionalized amine reactive group (NH2-C6)s at the 5′ terminal end of the sense strand to facilitate conjugation to the targeting ligand αvβ6 peptide 1. The myostatin RNAi agents further included a disulfide functional group (C6-SS-C6) at the 3′ terminal end of the sense strand to facilitate conjugation to a PK/PD modulator of structure LP29b, shown supra.

Two (2) cynos were dosed in each Group (n=2). Serum samples were taken on days −14, −7, and day 1 (pre-dose). Monkeys were then administered according to the respective Groups as set forth in Table 24. Serum was then collected on day 8, day 15, day 22, day 29, day 36, day 43, day 50, day 57, day 64, day 71, day 92, day 106 and day 120. An ELISA assay was performed on serum samples to determine the amount of cyno myostatin in serum. Average myostatin in serum samples is shown in Table 66 below.

TABLE 66
Average cyno myostatin protein in serum for dosing groups of
Example 21, normalized to Day 1.

| | Day-14 | | Day-7 | | Day 1 | | Day 8 | |
|---|---|---|---|---|---|---|---|---|
| | | Std | | Std | | Std | | Std |
| | Avg | Dev | Avg | Dev | Avg | Dev | Avg | Dev |
| Group 1 | 1.079 | 0.003 | 1.002 | 0.008 | 1.000 | 0.000 | 0.886 | 0.283 |
| (5 mpk) | | | | | | | | |
| Group 2 | 0.668 | 0.049 | 0.890 | 0.217 | 1.000 | 0.000 | 0.614 | 0.106 |
| (10 mpk) | | | | | | | | |
| Group 3 | 0.950 | 0.101 | 0.868 | 0.161 | 1.000 | 0.000 | 0.474 | 0.046 |
| (20 mpk) | | | | | | | | |
| | Day 15 | | Day 22 | | Day 29 | | Day 36 | |
| | | Std | | Std | | Std | | Std |
| | Avg | Dev | Avg | Dev | Avg | Dev | Avg | Dev |
| Group 1 | 0.842 | 0.014 | 0.856 | 0.035 | 0.706 | 0.183 | 0.791 | 0.035 |
| (5 mpk) | | | | | | | | |
| Group 2 | 0.700 | 0.175 | 0.542 | 0.165 | 0.620 | 0.032 | 0.500 | 0.072 |
| (10 mpk) | | | | | | | | |
| Group 3 | 0.540 | 0.150 | 0.328 | 0.027 | 0.298 | 0.053 | 0.227 | 0.023 |
| (20 mpk) | | | | | | | | |
| | Day 43 | | Day 50 | | Day 57 | | Day 64 | |
| | | Std | | Std | | Std | | Std |
| | Avg | Dev | Avg | Dev | Avg | Dev | Avg | Dev |
| Group 1 | 0.811 | 0.120 | 0.575 | 0.109 | 0.866 | 0.003 | 0.922 | 0.037 |
| (5 mpk) | | | | | | | | |
| Group 2 | 0.545 | 0.001 | 0.539 | 0.029 | 0.661 | 0.037 | 0.635 | 0.035 |
| (10 mpk) | | | | | | | | |
| Group 3 | 0.308 | 0.061 | 0.263 | 0.017 | 0.343 | 0.035 | 0.319 | 0.009 |
| (20 mpk) | | | | | | | | |
| | Day 71 | | Day 92 | | Day 106 | | Day 120 | |
| | | Std | | Std | | Std | | Std |
| | Avg | Dev | Avg | Dev | Avg | Dev | Avg | Dev |
| Group 1 | 0.717 | 0.090 | 0.922 | 0.196 | 1.033 | 0.281 | 0.772 | 0.163 |
| (5 mpk) | | | | | | | | |
| Group 2 | 0.462 | 0.036 | 0.553 | 0.052 | 0.801 | 0.021 | 0.559 | 0.085 |
| (10 mpk) | | | | | | | | |
| Group 3 | 0.316 | 0.001 | 0.471 | 0.026 | 0.510 | 0.057 | 0.353 | 0.014 |
| (20 mpk) | | | | | | | | |

As can be seen in Table 66, a dose-response effect is seen for increasing dosage of compounds of the present invention.

### Example 22. In Vivo Administration of RNAi Triggers Targeting MSTN in Rats

On Study Day 1, rats were injected with either isotonic saline (vehicle control) or 1 mg/kg (mpk) of a compound of the invention comprising an RNAi agent as described herein formulated in isotonic saline according to the following dosing Groups, wherein AD06569 has the structure shown in Table 24 above.

TABLE 67
Dosing Groups for Rats of Example 22.

| | Compound of Invention | |
|---|---|---|
| Group | Comprising RNAi Agent and Dose | Dosing Regimen |
| 1 | Saline | Single Injection on Day 1 |
| 2 | 1 mpk avb6-Pep1-AD06569-LP238b | Single Injection on Day 1 |
| 3 | 1 mpk avb6-Pep1-AD06569-LP357b | Single Injection on Day 1 |
| 4 | 1 mpk avb6-Pep1-AD06569-LP358b | Single Injection on Day 1 |
| 5 | 1 mpk avb6-Pep1-AD06569-LP241b | Single Injection on Day 1 |
| 6 | 1 mpk avb6-Pep1-AD06569-LP339b | Single Injection on Day 1 |
| 7 | 1 mpk avb6-Pep1-AD06569-LP340b | Single Injection on Day 1 |
| 8 | 1 mpk avb6-Pep1-AD06569-LP247b | Single Injection on Day 1 |
| 9 | 1 mpk avb6-Pep1-AD06569-nEm | Single Injection on Day 1 |

The RNAi agent AD06569 was synthesized having a nucleotide sequence targeted to the MSTN gene, and included a functionalized amine reactive group (NH2-C6)s at the 5′ terminal end of the sense strand to facilitate conjugation to the small molecule targeting ligand Compound 45b. The RNAi agent was also synthesized having a (C6-SS-C6) group on the 3′ end, to facilitate conjugation to a lipid PK/PD modulator precursor.

Groups 2-9 comprised an αvβ6 integrin ligand Peptide 1 conjugated to the 5′ end of the sense strand according to procedures described in Example 5, above. Each of groups 2-8 comprise a lipid PK/PD modulator, with structures as shown supra, conjugated to the 3′ end of the sense strand according to procedures described in Example 6, above. Group 3 included a capped maleimide conjugated to the 3′ end of the sense strand according to procedures described in Example 6, above.

Four (4) rats were dosed in each Group (n=4). Rats were bled and serum was collected on days 1, 8, 15 and 22. Rats were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on rat myostatin in serum. Average relative myostatin expression in serum is shown in Table 68 below.

TABLE 68
Average relative MSTN expression from serum for rats of
Example 22.

| | Day 8 | | Day 15 | | Day 22 | |
|---|---|---|---|---|---|---|
| Group | Avg | Std Dev | Avg | Std Dev | Avg | Std Dev |
| 1. Saline | 1.000 | 0.068 | 1.000 | 0.038 | 1.000 | 0.087 |
| 2. 1 mpk avb6-Pep1- | 0.804 | 0.012 | 0.679 | 0.129 | 0.785 | 0.017 |
| AD06569-LP238b | | | | | | |
| 3. 1 mpk avb6-Pep1- | 0.665 | 0.061 | 0.718 | 0.024 | 0.814 | 0.031 |
| AD06569-LP357b | | | | | | |
| 4. 1 mpk avb6-Pep1- | 0.683 | 0.063 | 0.747 | 0.143 | 0.792 | 0.104 |
| AD06569-LP358b | | | | | | |
| 5. 1 mpk avb6-Pep1- | 0.723 | 0.052 | 0.826 | 0.066 | 0.862 | 0.086 |
| AD06569-LP241b | | | | | | |
| 6. 1 mpk avb6-Pep1- | 0.749 | 0.044 | 0.898 | 0.082 | 0.889 | 0.054 |
| AD06569-LP339b | | | | | | |
| 7. 1 mpk avb6-Pep1- | 0.764 | 0.184 | 0.726 | 0.129 | 0.729 | 0.155 |
| AD06569-LP340b | | | | | | |
| 8. 1 mpk avb6-Pep1- | 0.803 | 0.082 | 0.709 | 0.091 | 0.691 | 0.050 |
| AD06569-LP247b | | | | | | |
| 9. 1 mpk avb6-Pep1- | 0.825 | 0.086 | 0.778 | 0.124 | 0.836 | 0.110 |
| AD06569-nEm | | | | | | |

Tissue collected from the gastrocnemius and triceps was used in a TaqMan assay to determine the relative amounts of MSTN in those tissues. Table 69, below, shows the results of the assay.

TABLE 69
Relative Expression in Triceps and Gastrocnemius in
dosing groups of Example 22.

| | Triceps | | | Gastrocnemius | | |
|---|---|---|---|---|---|---|
| | Rel. | Low | High | Rel. | Low | High |
| Group | Exp. | Error | Error | Exp. | Error | Error |
| 1. Saline | 1.000 | 0.884 | 7.639 | 1.000 | 0.109 | 0.123 |
| 2. 1 mpk avb6-Pep1- | 2.650 | 1.962 | 7.558 | 0.709 | 0.073 | 0.081 |
| AD06569-LP238b | | | | | | |
| 3. 1 mpk avb6-Pep1- | 1.055 | 0.826 | 3.809 | 0.768 | 0.164 | 0.208 |
| AD06569-LP357b | | | | | | |
| 4. 1 mpk avb6-Pep1- | 1.603 | 1.249 | 5.654 | 0.740 | 0.140 | 0.173 |
| AD06569-LP358b | | | | | | |
| 5. 1 mpk avb6-Pep1- | 3.585 | 2.698 | 10.907 | 0.927 | 0.107 | 0.121 |
| AD06569-LP241b | | | | | | |
| 6. 1 mpk avb6-Pep1- | 7.246 | 1.597 | 2.049 | 0.710 | 0.134 | 0.165 |
| AD06569-LP339b | | | | | | |
| 7. 1 mpk avb6-Pep1- | 7.104 | 1.987 | 2.759 | 0.708 | 0.124 | 0.150 |
| AD06569-LP340b | | | | | | |
| 8. 1 mpk avb6-Pep1- | 5.038 | 0.471 | 0.520 | 0.719 | 0.103 | 0.121 |
| AD06569-LP247b | | | | | | |
| 9. 1 mpk avb6-Pep1- | 5.698 | 1.786 | 2.602 | 0.676 | 0.141 | 0.178 |
| AD06569-nEm | | | | | | |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of Formula (I): 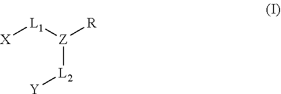
or a pharmaceutically acceptable salt thereof, wherein
R is -L_{A}-R_{Z};
L_{A} is a bond or a bivalent moiety connecting R_{Z} to Z;
R_{Z} comprises an oligonucleotide-based agent;
Z is CH, phenyl or N;
L₁ and L₂ are each independently linkers comprising at least about 5 PEG units; and
X and Y are each independently lipids comprising from about 10 to about 50 carbon atoms.

2. The compound or pharmaceutically acceptable salt of claim 1, wherein L₁ and L₂ each independently comprise about 15 to about 100 PEG units.

3. The compound or pharmaceutically acceptable salt of claim 1 or claim 2, wherein L₁ and L₂ each independently comprise about 20 to about 60 PEG units.

4. The compound or pharmaceutically acceptable salt of any one of claims 1-3, wherein L₁ and L₂ each independently comprise about 20 to about 30 PEG units.

5. The compound or pharmaceutically acceptable salt of any one of claims 1-3, wherein L₁ and L₂ each independently comprise about 40 to about 60 PEG units.

6. The compound or pharmaceutically acceptable salt of claim 1, wherein one of L₁ and L₂ comprises about 20 to about 30 PEG units and the other comprises about 40 to about 60 PEG units.

7. The compound or pharmaceutically acceptable salt of claim 1, wherein each of L₁ and L₂ is independently selected from the group consisting of:

| Name | Structure |
|---|---|
| Linker 1 | 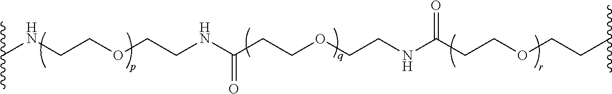 |
| Linker 2 | 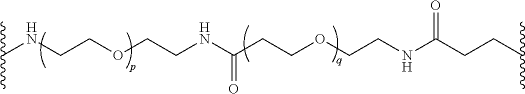 |
| Linker 3 | 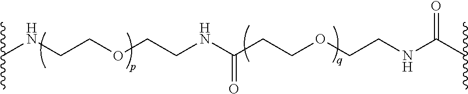 |
| Linker 4 | 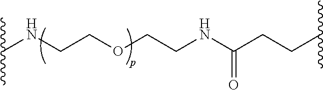 |
| Linker 5 | 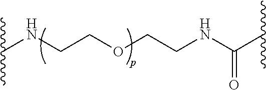 |
| Linker 6 | 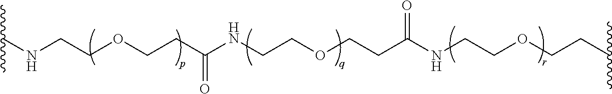 |
| Linker 7 | 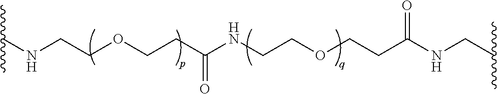 |
| Linker 8 | 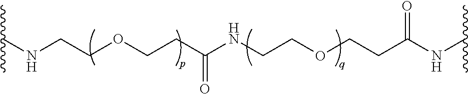 |
| Linker 9 | 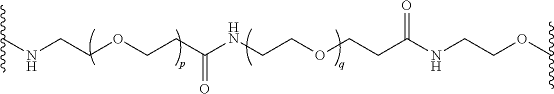 |
| Linker 10 | 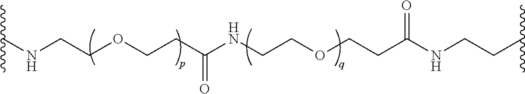 |
| Linker 11 | 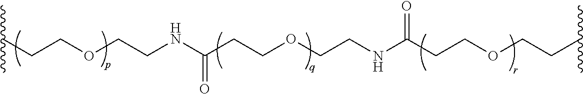 |
| Linker 12 | 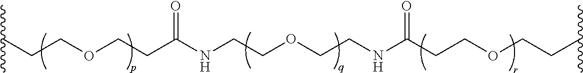 |

wherein,
each p is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30;
each q is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30;
each r is independently 1, 2, 3, 4, 5, 6, 7, 8, 9, or 10; and
each [symbol] indicates a point of connection to X, Y, or Z; provided that
(i) in Linker 1, 6, and 11, p+q+r≥5;
(ii) in Linker 2, 3, 7, 8, 9, and 10, p+q≥5; and
(iii) in Linker 4 and 5, p≥5.

8. The compound or pharmaceutically acceptable salt of claim 7, wherein
each p is independently 20, 21, 22, 23, 24, or 25;
each q is independently 20, 21, 22, 23, 24, or 25; and
each r is independently 2, 3, 4, 5, or 6.

9. The compound or pharmaceutically acceptable salt of claim 1, wherein the compound of Formula (I) is a compound of Formula (Ia): 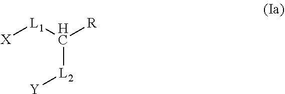
or a pharmaceutically acceptable salt thereof.

10. The compound or pharmaceutically acceptable salt of claim 1, wherein the compound of Formula (I) is a compound of Formula (Ib): 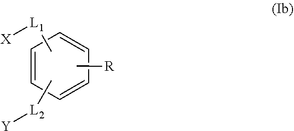
or a pharmaceutically acceptable salt thereof.

11. The compound or pharmaceutically acceptable salt of claim 1, wherein the compound of Formula (I) is a compound of Formula (Ic): 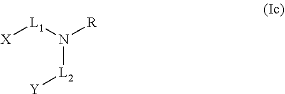
or a pharmaceutically acceptable salt thereof.

12. The compound or pharmaceutically acceptable salt of any one of claims 1-11, wherein at least one of X and Y is an unsaturated lipid.

13. The compound or pharmaceutically acceptable salt of any one of claims 1-12, wherein at least one of X and Y is a saturated lipid.

14. The compound or pharmaceutically acceptable salt of any one of claims 1-13, wherein at least one of X and Y is a branched lipid.

15. The compound or pharmaceutically acceptable salt of any one of claims 1-14, wherein at least one of X and Y is a straight chain lipid.

16. The compound or pharmaceutically acceptable salt of any one of claims 1-15, wherein at least one of X and Y is a lipid comprising from about 10 to about 25 carbon atoms.

17. The compound or pharmaceutically acceptable salt of any one of claims 1-16, wherein at least one of X and Y is cholesteryl.

18. The compound or pharmaceutically acceptable salt of any one of claims 1-11, wherein at least one of X and Y is selected from the group consisting of:

| Name | Structure |
|---|---|
| Lipid 1 | 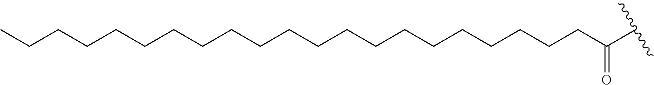 [SMILES: *C(=O)CCCCCCCCCCCCCCCCCCCCC] |
| Lipid 2 | 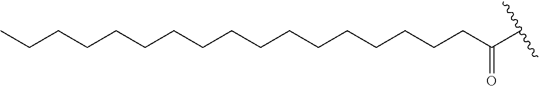 [SMILES: *C(=O)CCCCCCCCCCCCCCCCC] |
| Lipid 3 | 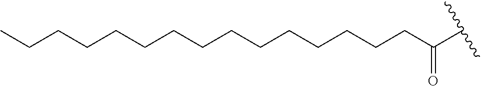 [SMILES: *C(=O)CCCCCCCCCCCCCCC] |
| Lipid 4 | 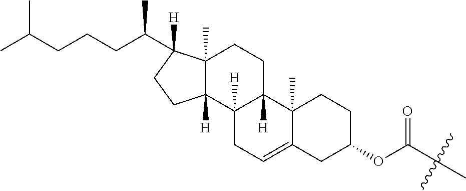 |
| Lipid 5 | 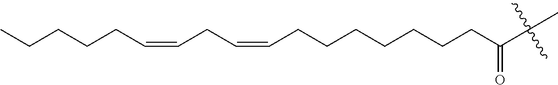 [SMILES: *C(=O)CCCCCCC/C=C\C/C=C\CCCCC] |
| Lipid 6 | 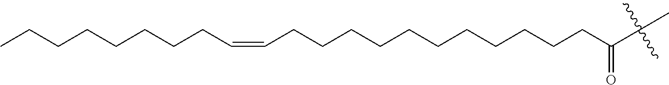 [SMILES: *C(=O)CCCCCCCCCCC/C=C\CCCCCCCC] |
| Lipid 7 | 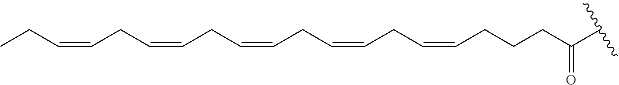 [SMILES: *C(=O)CCC/C=C\C/C=C\C/C=C\C/C=C\C/C=C\CC] |
| Lipid 8 | 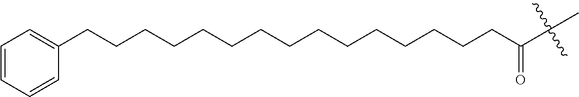 [SMILES: *C(=O)CCCCCCCCCCCCCCCc1ccccc1] |
| Lipid 9 | 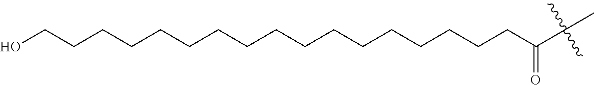 [SMILES: *C(=O)CCCCCCCCCCCCCCCCCO] |
| Lipid 10 | 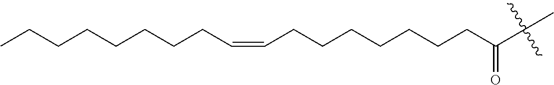 [SMILES: *C(=O)CCCCCCC/C=C\CCCCCCCC] |
| Lipid 11 | 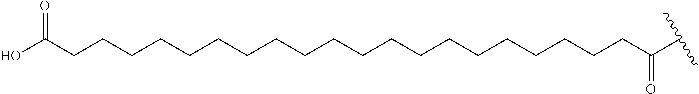 [SMILES: *C(=O)CCCCCCCCCCCCCCCCCCCCC(=O)O] |
| Lipid 12 | 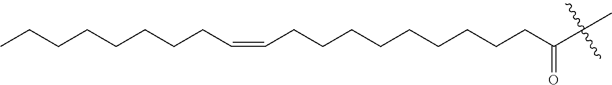 [SMILES: *C(=O)CCCCCCCCC/C=C\CCCCCCCC] |
| Lipid 14 | 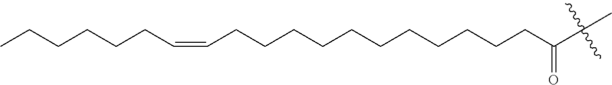 [SMILES: *C(=O)CCCCCCCCCCC/C=C\CCCCCC] |
| Lipid 15 | 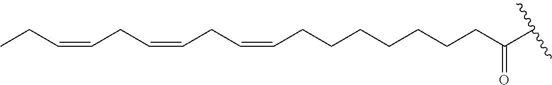 [SMILES: *C(=O)CCCCCCC/C=C\C/C=C\C/C=C\CC] |
| Lipid 16 | 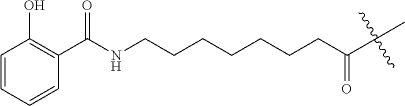 [SMILES: *C(=O)CCCCCCCNC(=O)c1ccccc1O] |
| Lipid 17 | 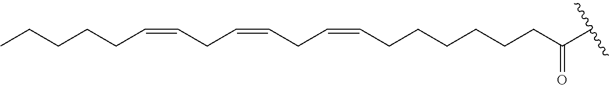 [SMILES: *C(=O)CCCCCC/C=C\C/C=C\C/C=C\CCCCC] |
| Lipid 18 | 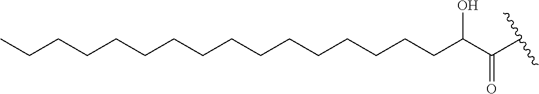 [SMILES: *C(=O)C(O)CCCCCCCCCCCCCCCC] |
| Lipid 19 | 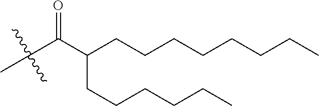 [SMILES: *C(=O)C(CCCCCC)CCCCCCCC] |
| Lipid 20 | 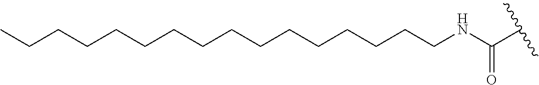 [SMILES: *C(=O)NCCCCCCCCCCCCCCCC] |
| Lipid 21 | 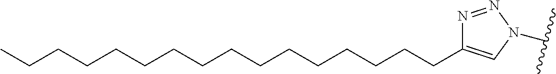 [SMILES: *n1cc(CCCCCCCCCCCCCCCC)nn1] |
| Lipid 22 | 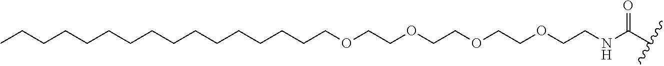 [SMILES: *C(=O)NCCOCCOCCOCCOCCCCCCCCCCCCCCCC] |
| Lipid 23 | 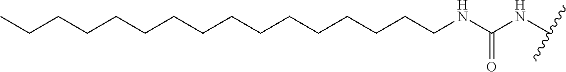 [SMILES: *NC(=O)NCCCCCCCCCCCCCCCC] |
| Lipid 24 | 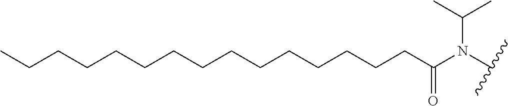 [SMILES: *N(C(=O)CCCCCCCCCCCCCCC)C(C)C] |

wherein, [symbol] indicates a point of connection to L₁ or L₂.

19. The compound or pharmaceutically acceptable salt of any one of claims 1-11, wherein each of X and Y are independently selected from the group consisting of:

| Name | Structure |
|---|---|
| Lipid 1 | 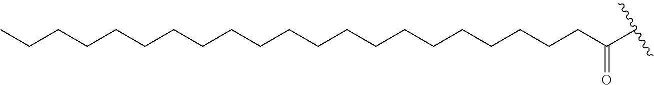 [SMILES: *C(=O)CCCCCCCCCCCCCCCCCCCCC] |
| Lipid 2 | 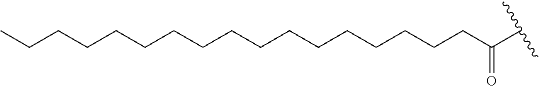 [SMILES: *C(=O)CCCCCCCCCCCCCCCCC] |
| Lipid 3 | 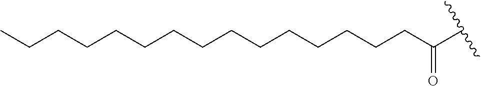 [SMILES: *C(=O)CCCCCCCCCCCCCCC] |
| + | |
| Lipid 4 | 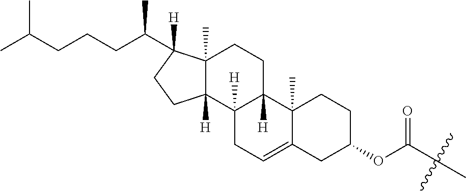 |
| Lipid 5 | 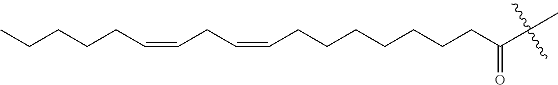 [SMILES: *C(=O)CCCCCCC/C=C\C/C=C\CCCCC] |
| Lipid 6 | 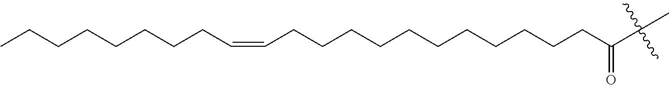 [SMILES: *C(=O)CCCCCCCCCCC/C=C\CCCCCCCC] |
| Lipid 7 | 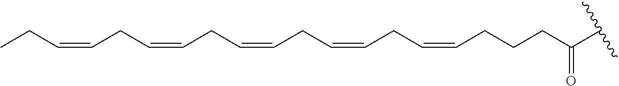 [SMILES: *C(=O)CCC/C=C\C/C=C\C/C=C\C/C=C\C/C=C\CC] |
| Lipid 8 | 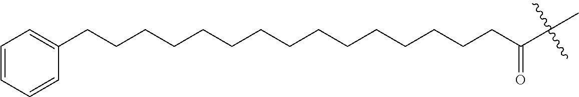 [SMILES: *C(=O)CCCCCCCCCCCCCCCc1ccccc1] |
| Lipid 9 | 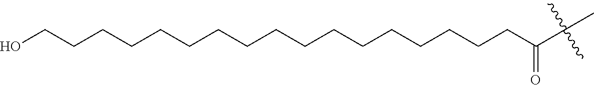 [SMILES: *C(=O)CCCCCCCCCCCCCCCCCO] |
| Lipid 10 | 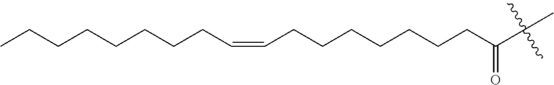 [SMILES: *C(=O)CCCCCCC/C=C\CCCCCCCC] |
| Lipid 11 | 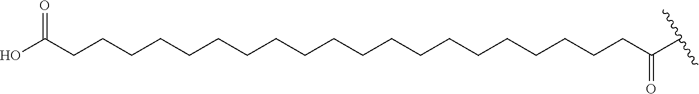 [SMILES: *C(=O)CCCCCCCCCCCCCCCCCCCCC(=O)O] |
| Lipid 12 | 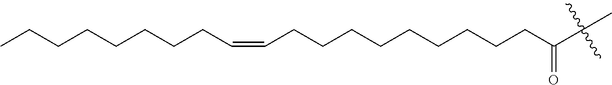 [SMILES: *C(=O)CCCCCCCCC/C=C\CCCCCCCC] |
| Lipid 14 | 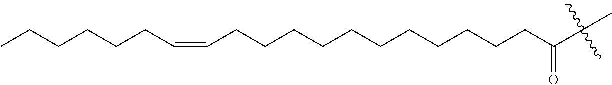 [SMILES: *C(=O)CCCCCCCCCCC/C=C\CCCCCC] |
| Lipid 15 | 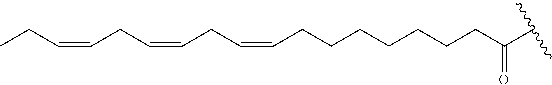 [SMILES: *C(=O)CCCCCCC/C=C\C/C=C\C/C=C\CC] |
| Lipid 16 | 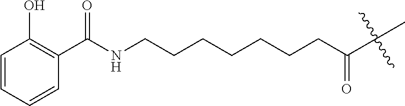 [SMILES: *C(=O)CCCCCCCNC(=O)c1ccccc1O] |
| Lipid 17 | 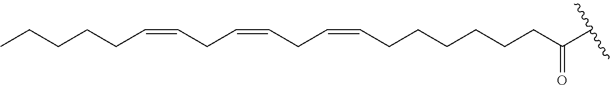 [SMILES: *C(=O)CCCCCC/C=C\C/C=C\C/C=C\CCCCC] |
| Lipid 18 | 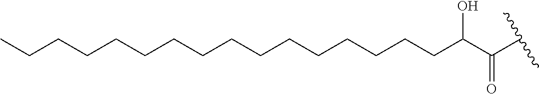 [SMILES: *C(=O)C(O)CCCCCCCCCCCCCCCC] |
| Lipid 19 | 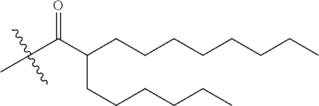 [SMILES: *C(=O)C(CCCCCC)CCCCCCCC] |
| Lipid 20 | 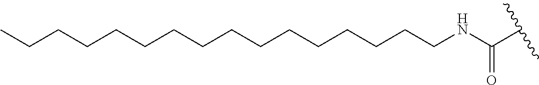 [SMILES: *C(=O)NCCCCCCCCCCCCCCCC] |
| Lipid 21 | 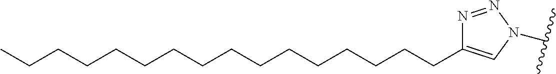 [SMILES: *n1cc(CCCCCCCCCCCCCCCC)nn1] |
| Lipid 22 | 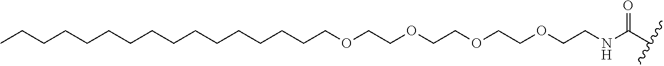 [SMILES: *C(=O)NCCOCCOCCOCCOCCCCCCCCCCCCCCCC] |
| Lipid 23 | 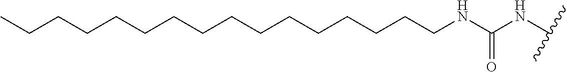 [SMILES: *NC(=O)NCCCCCCCCCCCCCCCC] |
| Lipid 24 | 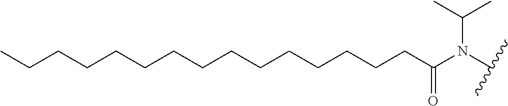 [SMILES: *N(C(=O)CCCCCCCCCCCCCCC)C(C)C] |

wherein [symbol] indicates a point of connection to L₁ or L₂.

20. The compound or pharmaceutically acceptable salt of claim 1, wherein L_{A} is selected from the group consisting of:

| Name | Structure |
|---|---|
| Tether 1 | 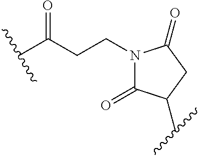 [SMILES: *C(=O)CCN1C(=O)CC(*)C1=O] |
| Tether 2 | 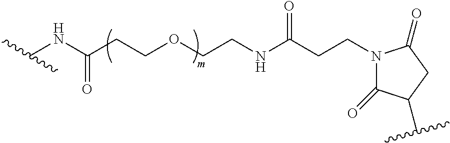 |
| Tether 3 | 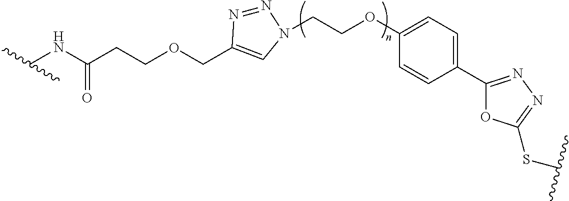 |
| Tether 4 | 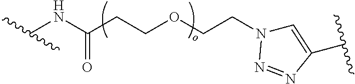 |
| Tether 5 | 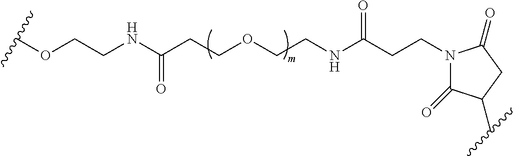 |
| Tether 6 | 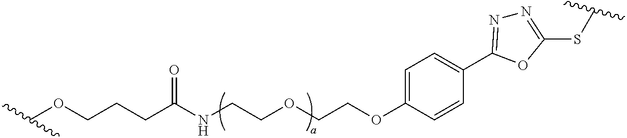 |
| Tether 7 | 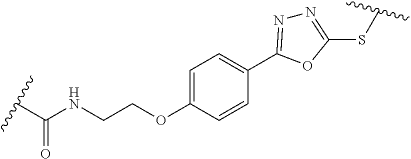 [SMILES: *Sc1nnc(-c2ccc(OCCNC(*)=O)cc2)o1] |
| Tether 8 | 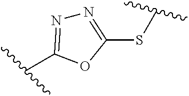 [SMILES: *Sc1nnc(*)o1] |
| Tether 9 | 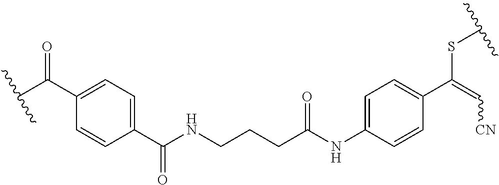 [SMILES: *SC(=CC#N)c1ccc(NC(=O)CCCNC(=O)c2ccc(C(*)=O)cc2)cc1] |
| Tether 10 | 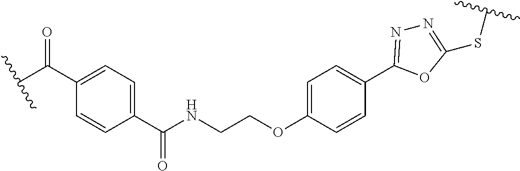 [SMILES: *Sc1nnc(-c2ccc(OCCNC(=O)c3ccc(C(*)=O)cc3)cc2)o1] |
| Tether 11 | 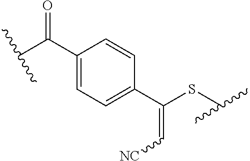 [SMILES: *SC(=CC#N)c1ccc(C(*)=O)cc1] |
| Tether 12 | 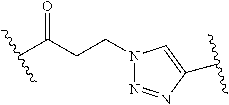 [SMILES: *C(=O)CCn1cc(*)nn1] |
| Tether 13 | 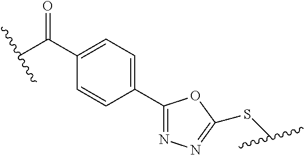 [SMILES: *Sc1nnc(-c2ccc(C(*)=O)cc2)o1] |
| Tether 14 | 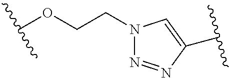 [SMILES: *OCCn1cc(*)nn1] |

wherein, each of m, n, o, and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to Z or R_{Z}.

21. The compound or pharmaceutically acceptable salt of claim 20 wherein,
each m is independently 1, 2, 3, 4, 5, 6, 7, 8, 8, 9, 10, 20, 21, 22, 23, 24, or 25;
each n is independently 2, 3, 4, or 5;
each a is independently 2, 3, or 4; and
each o is independently 2, 3, 4, 5, 6, 7, 8, 9, 10, 11, 12, or 13.

22. The compound or pharmaceutically acceptable salt of claim 9, wherein L₁ and L₂ are independently selected from the group consisting of 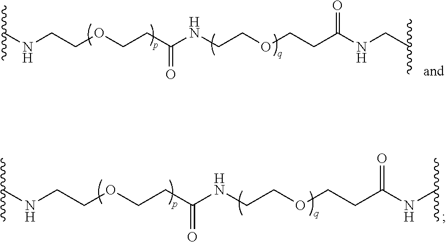
wherein each p is independently 20, 21, 22, 23, 24, or 25; each q is independently 20, 21, 22, 23, 24, or 25; and each [symbol] indicates a point of connection to X, Y, or CH of Formula (Ia).

23. The compound or pharmaceutically acceptable salt of claim 22, wherein L_{A} is 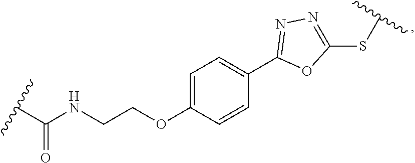 [SMILES: *Sc1nnc(-c2ccc(OCCNC(*)=O)cc2)o1]
and each [symbol] indicates a point of connection to R_{Z} or CH of Formula (Ia).

24. The compound or pharmaceutically acceptable salt of claim 22 or claim 23, wherein each of X and Y are 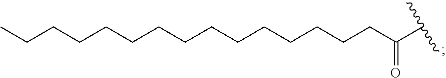 [SMILES: *C(=O)CCCCCCCCCCCCCCC]
and [symbol]
indicates a point of connection to L₁ or L₂.

25. A compound selected from the group consisting of: 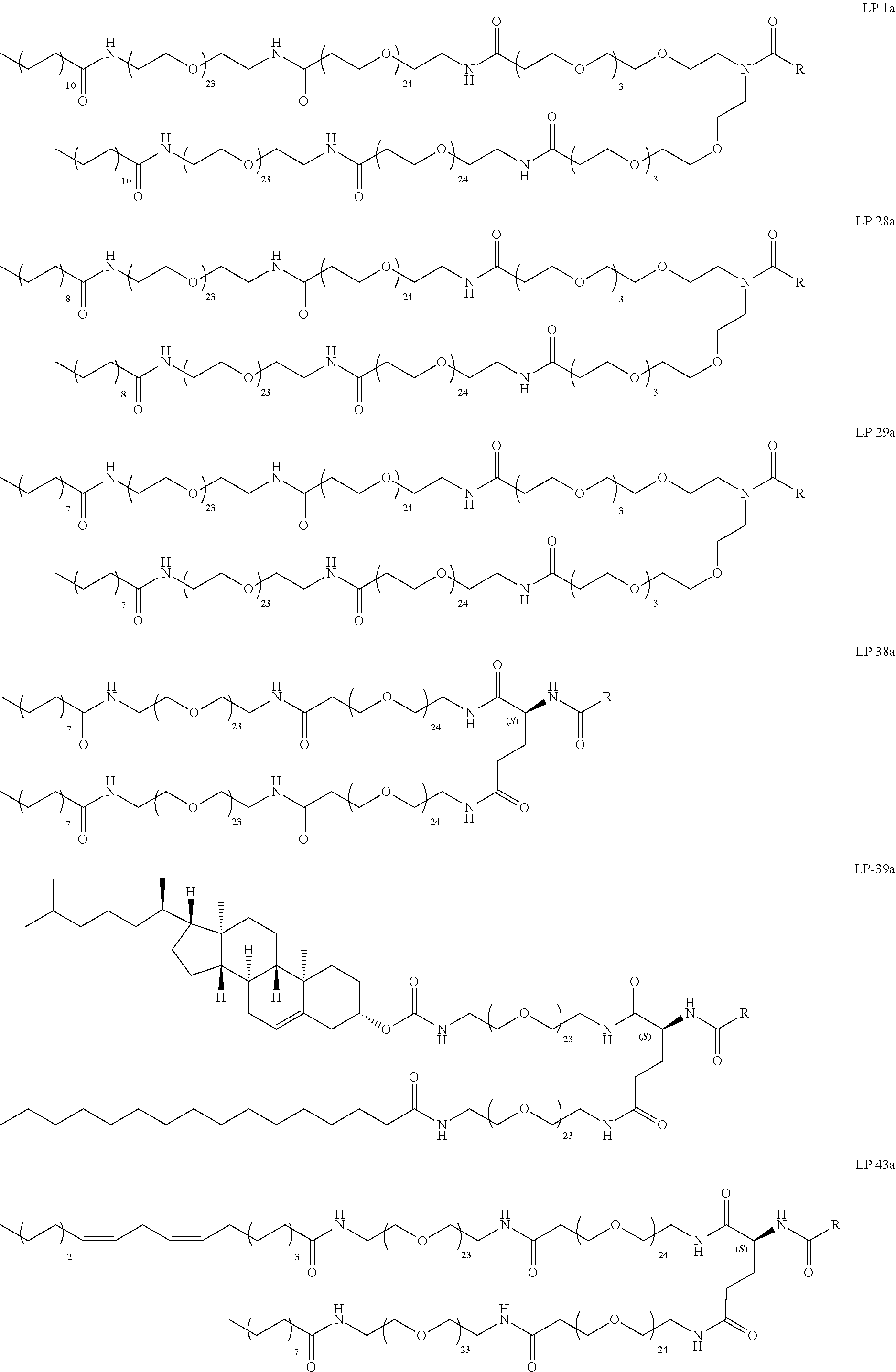 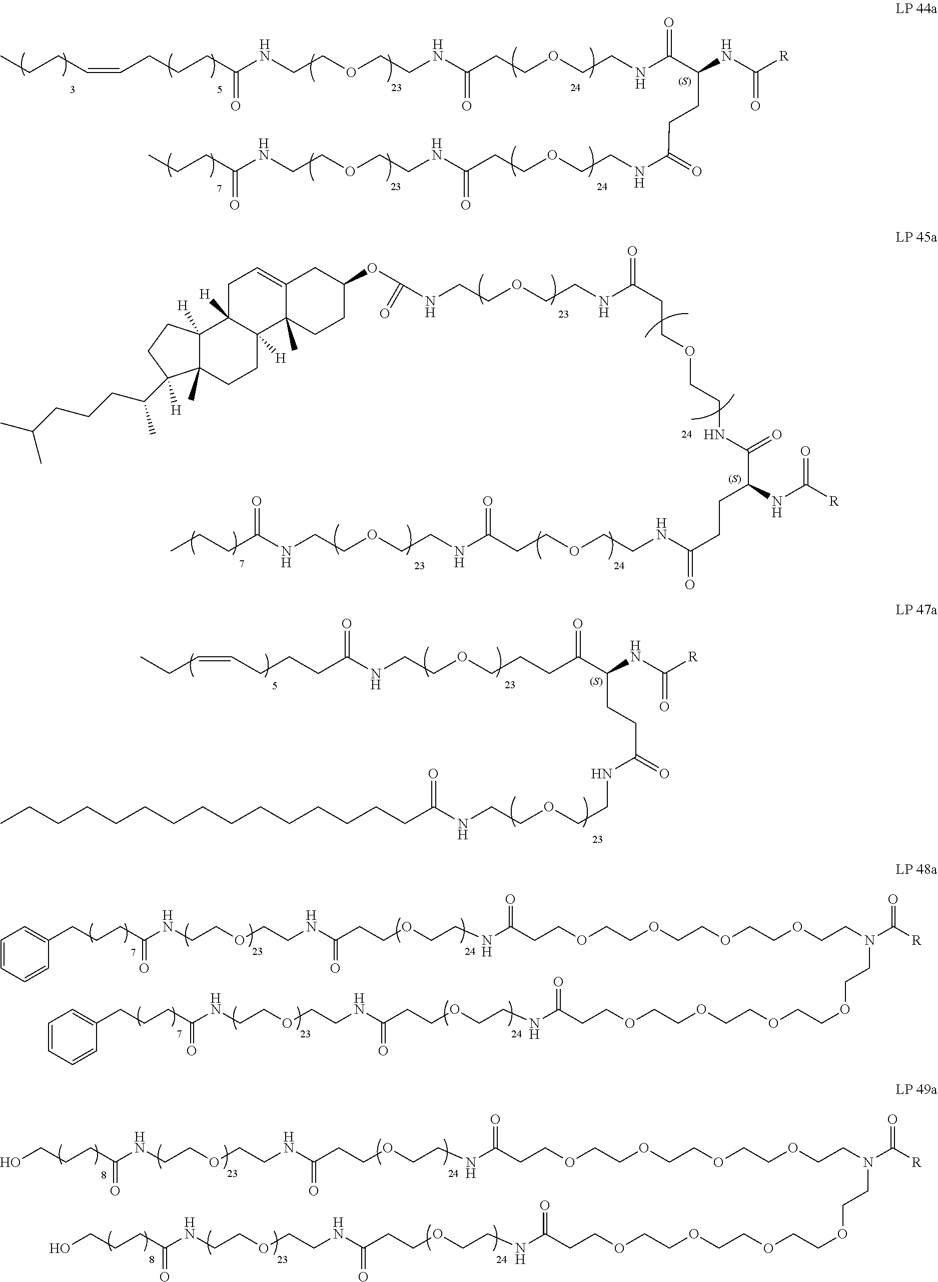 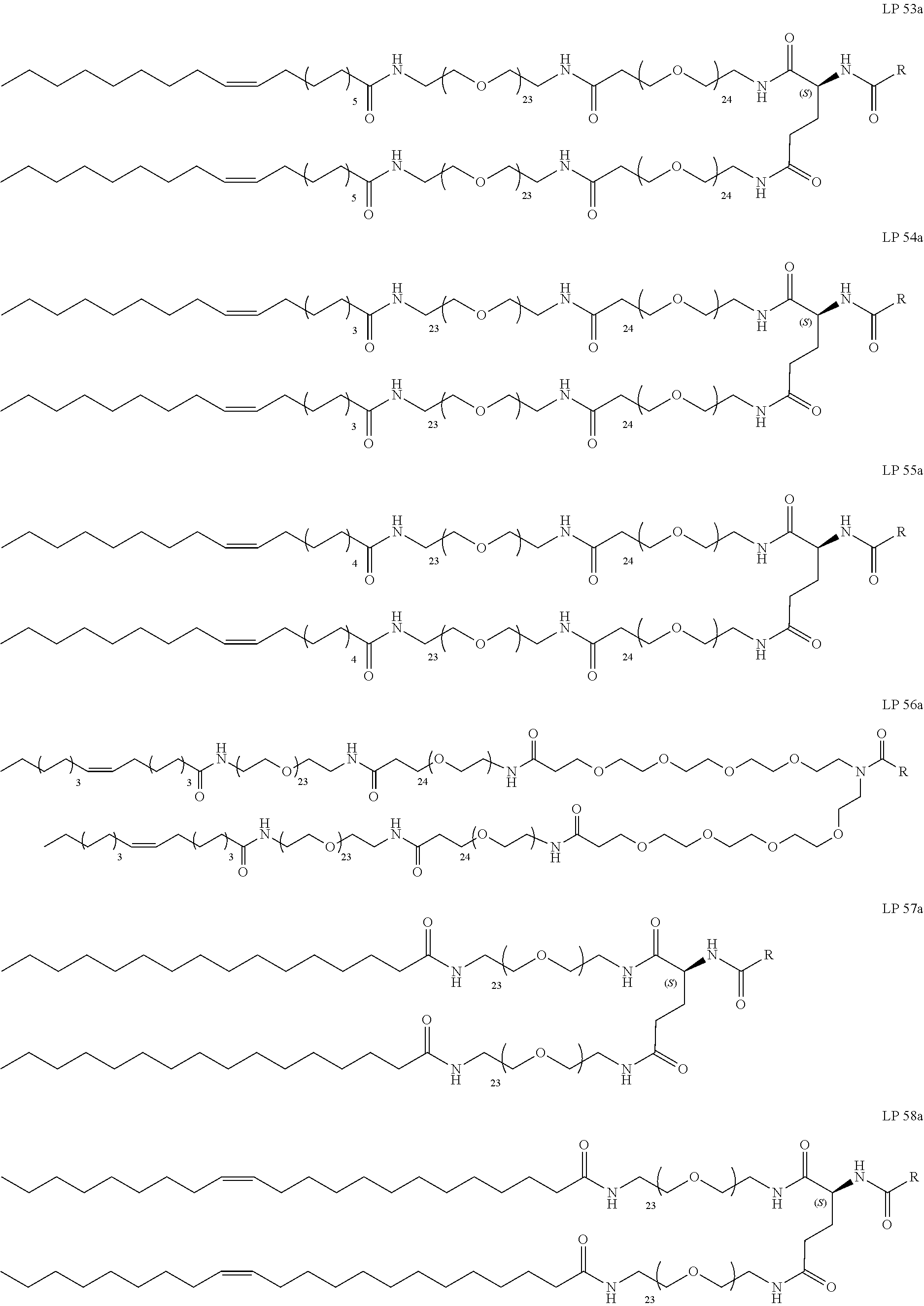 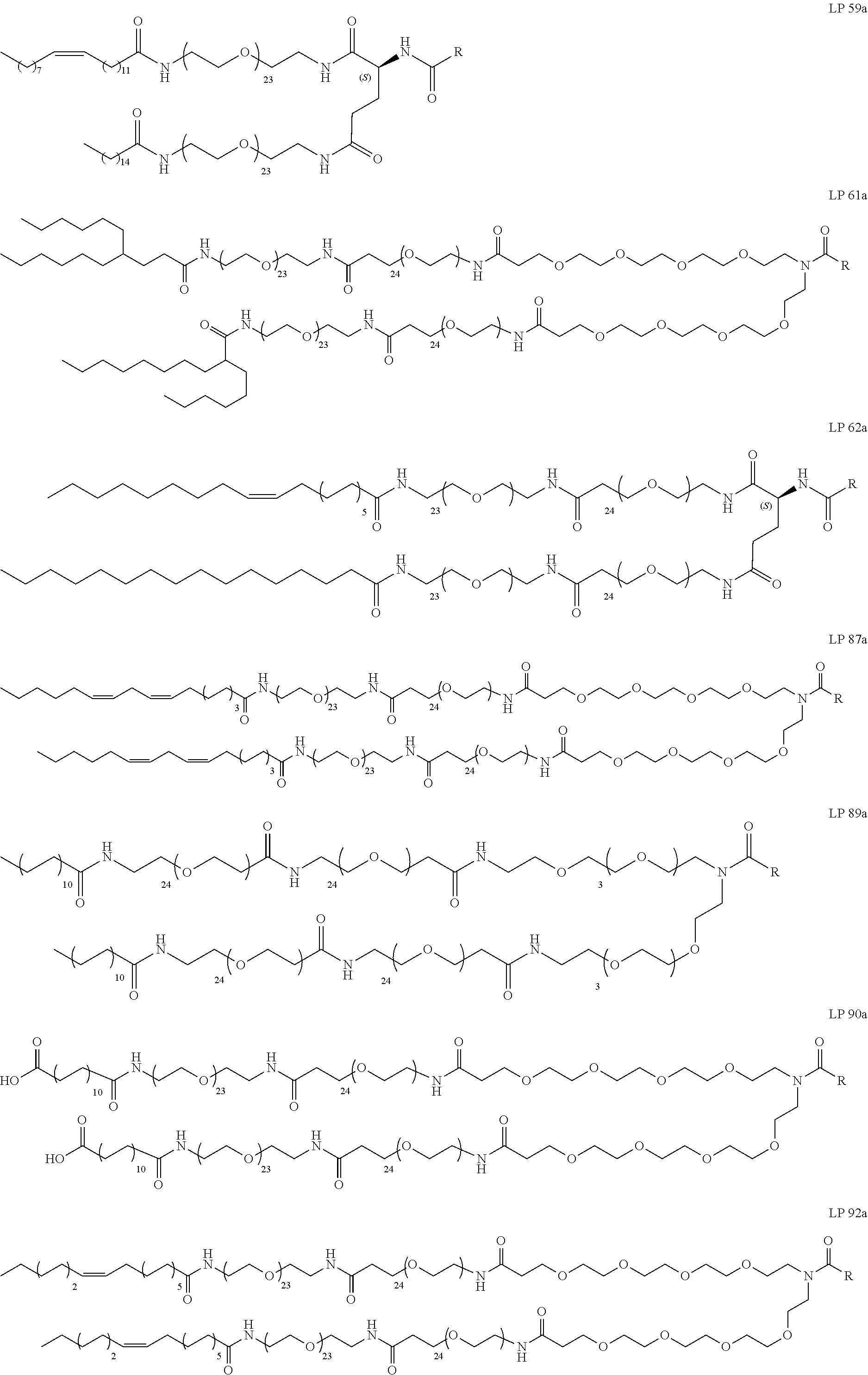 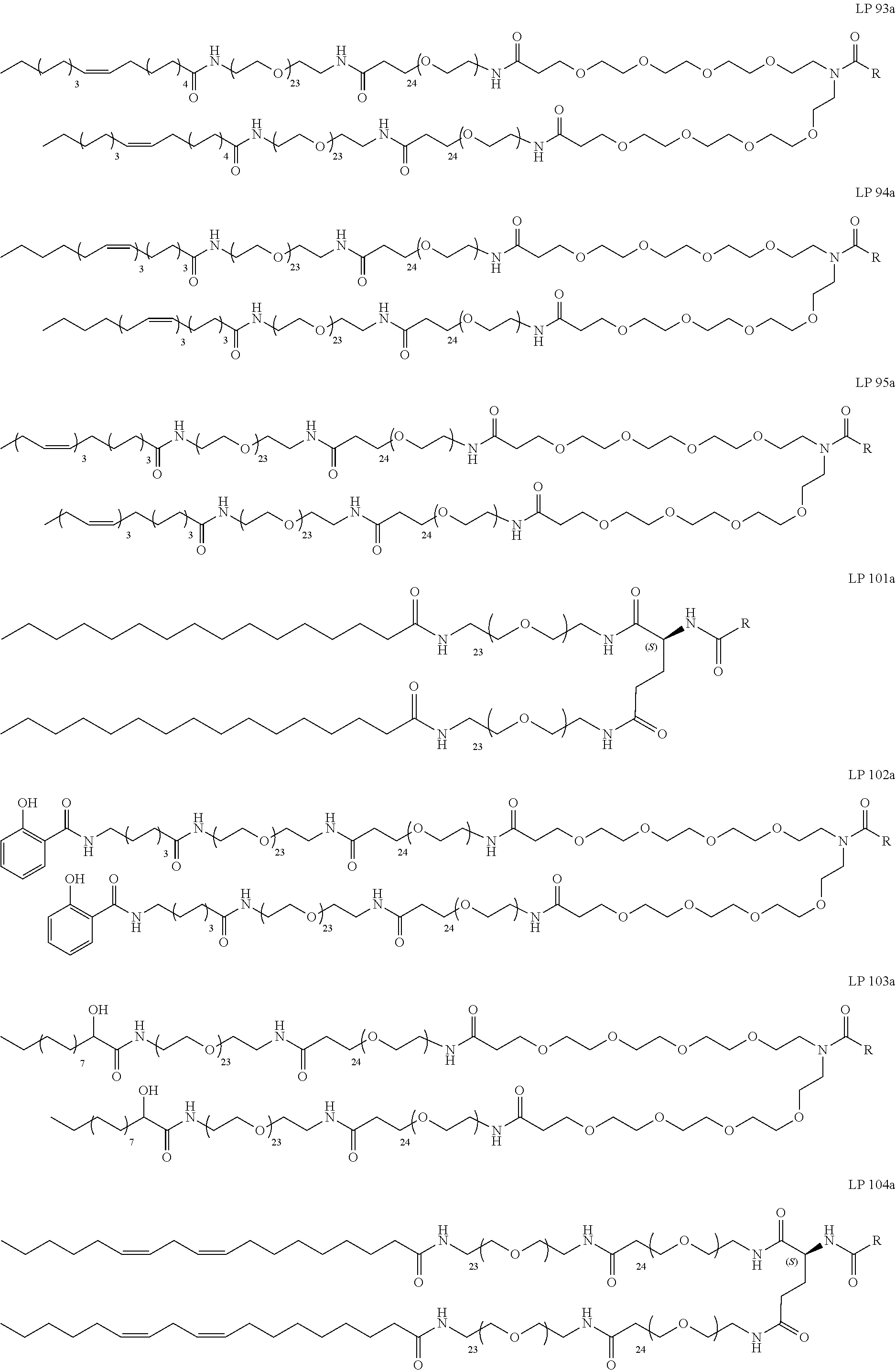 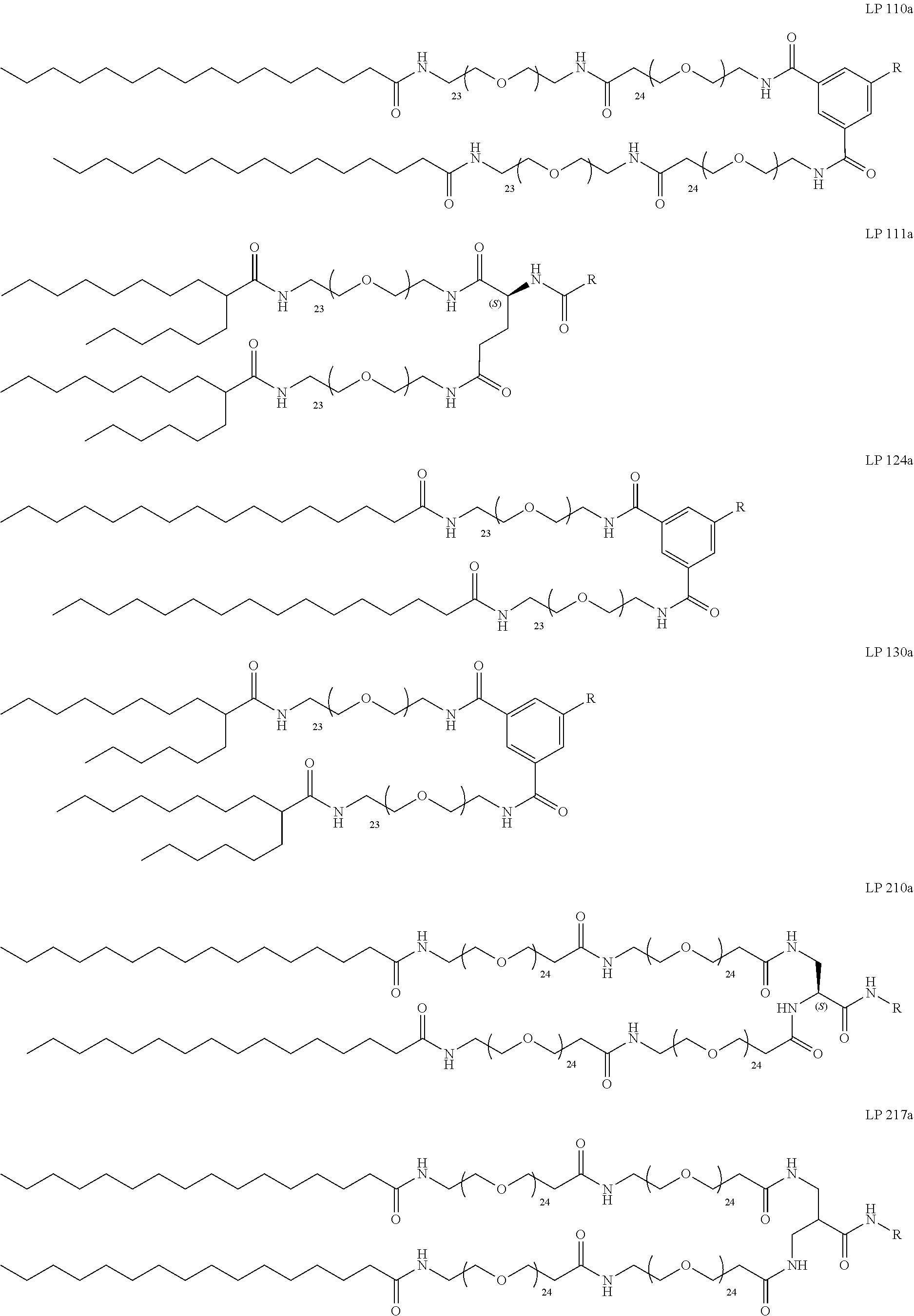 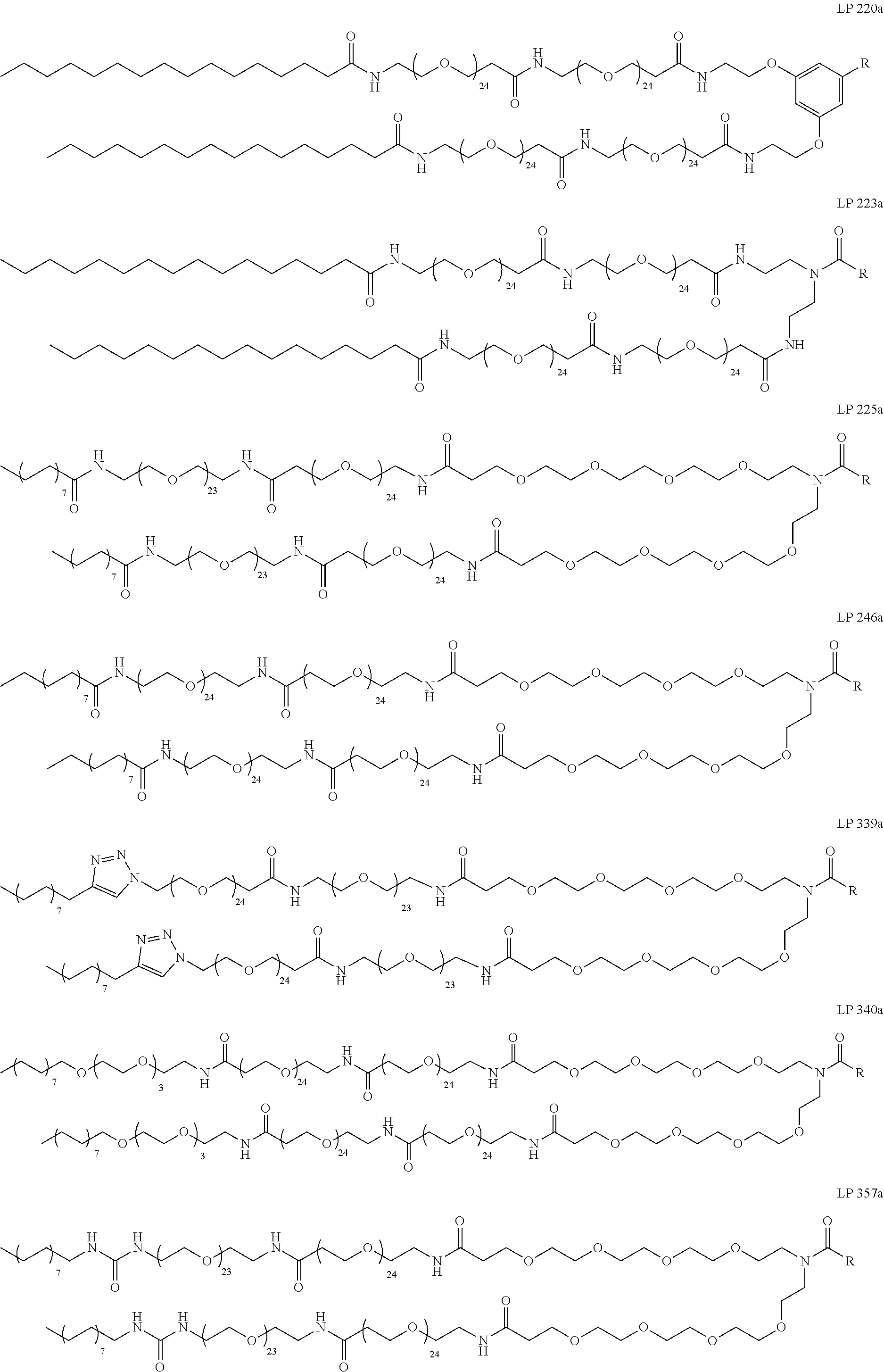 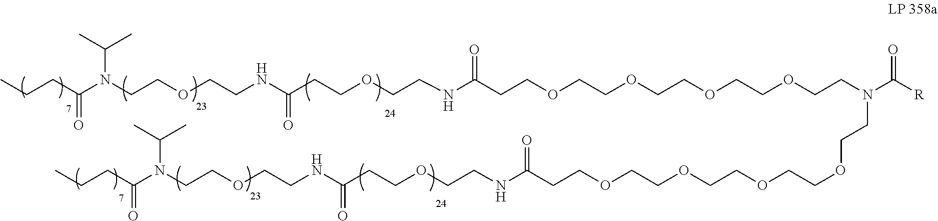
or a pharmaceutically acceptable salt ofany of these compounds,
wherein each R is L_{A}-R_{Z};
L_{A} is a bond or a bivalent moiety connecting R_{Z} to the rest of the compound; and
R_{Z} is an oligonucleotide-based agent.

26. A compound selected from: 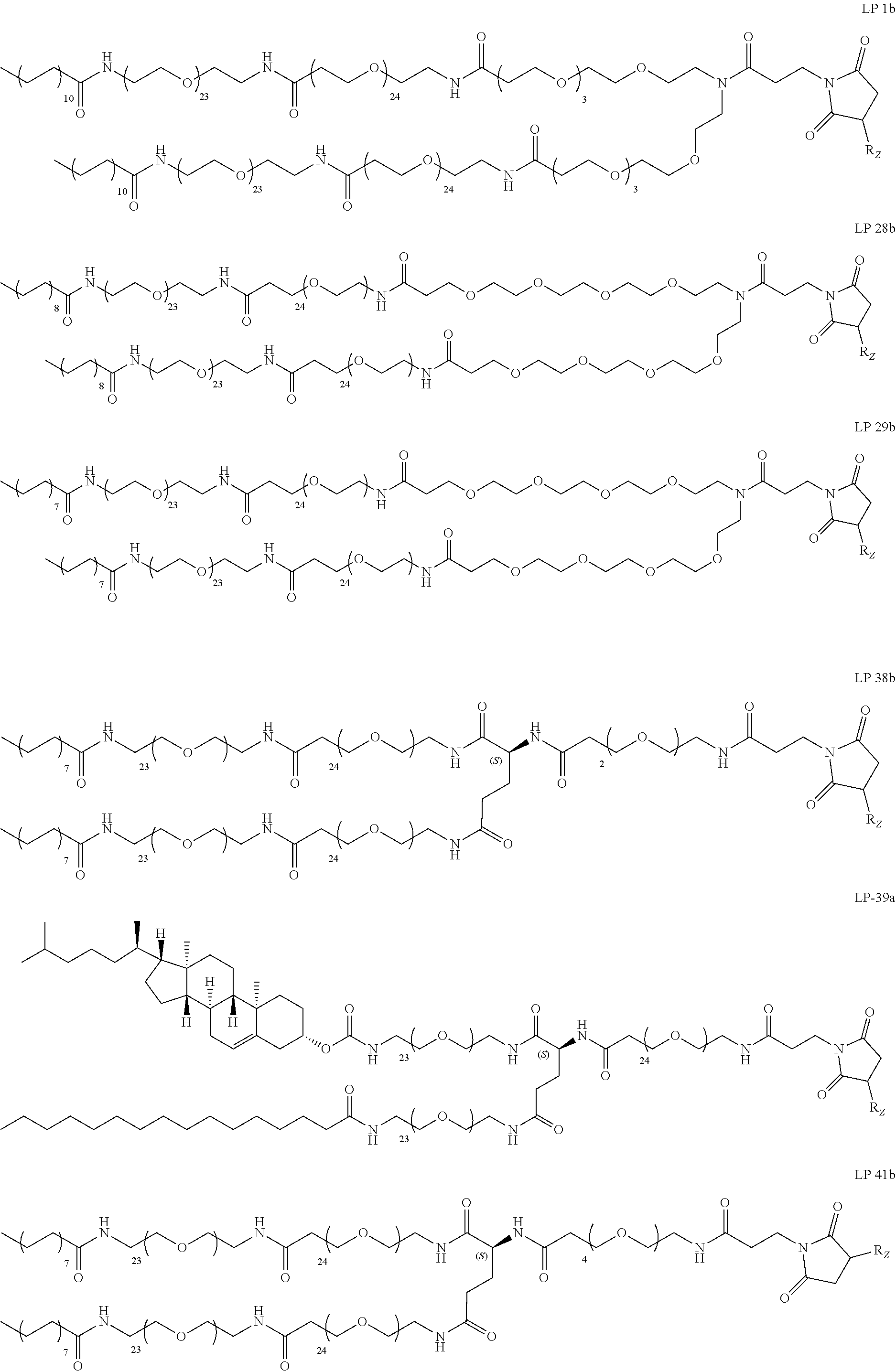 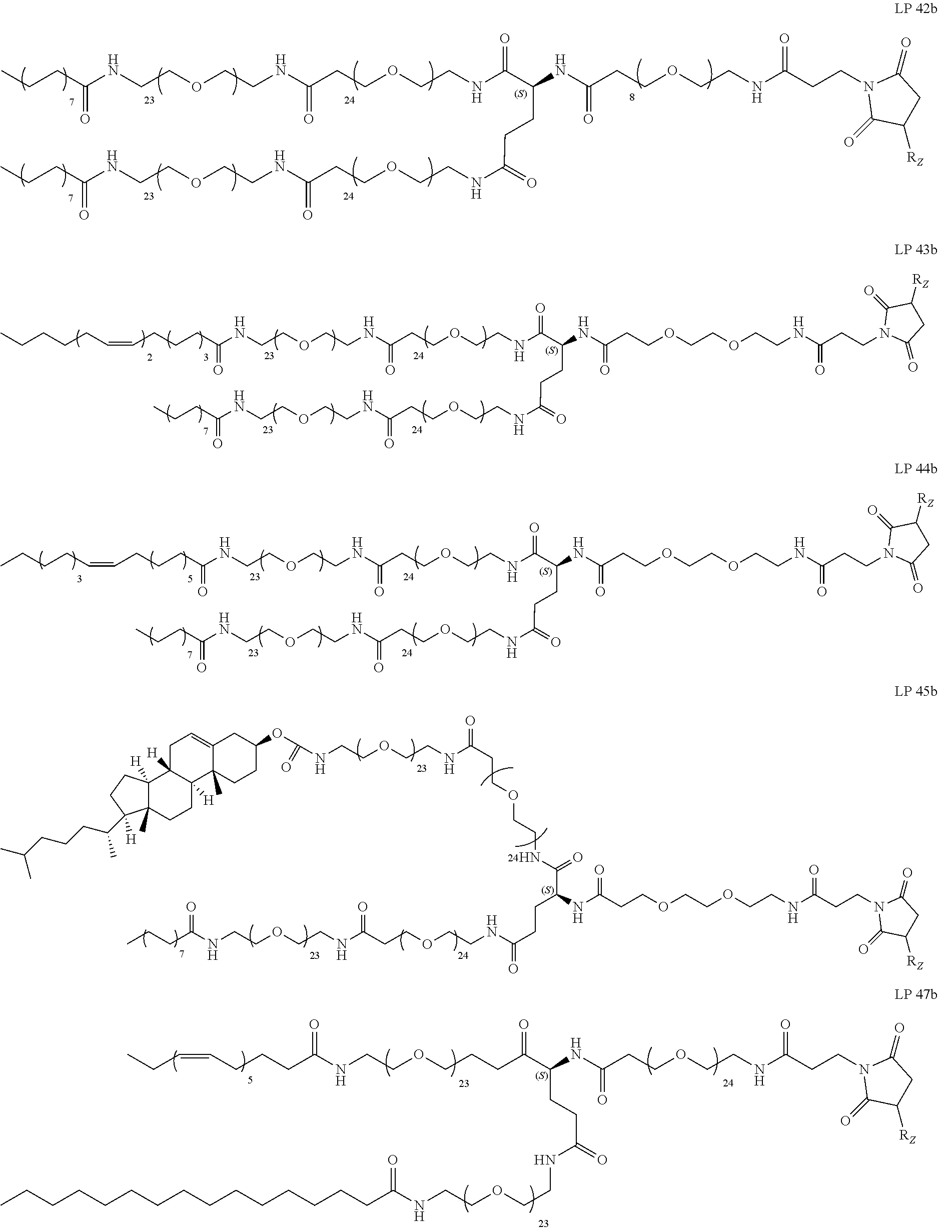 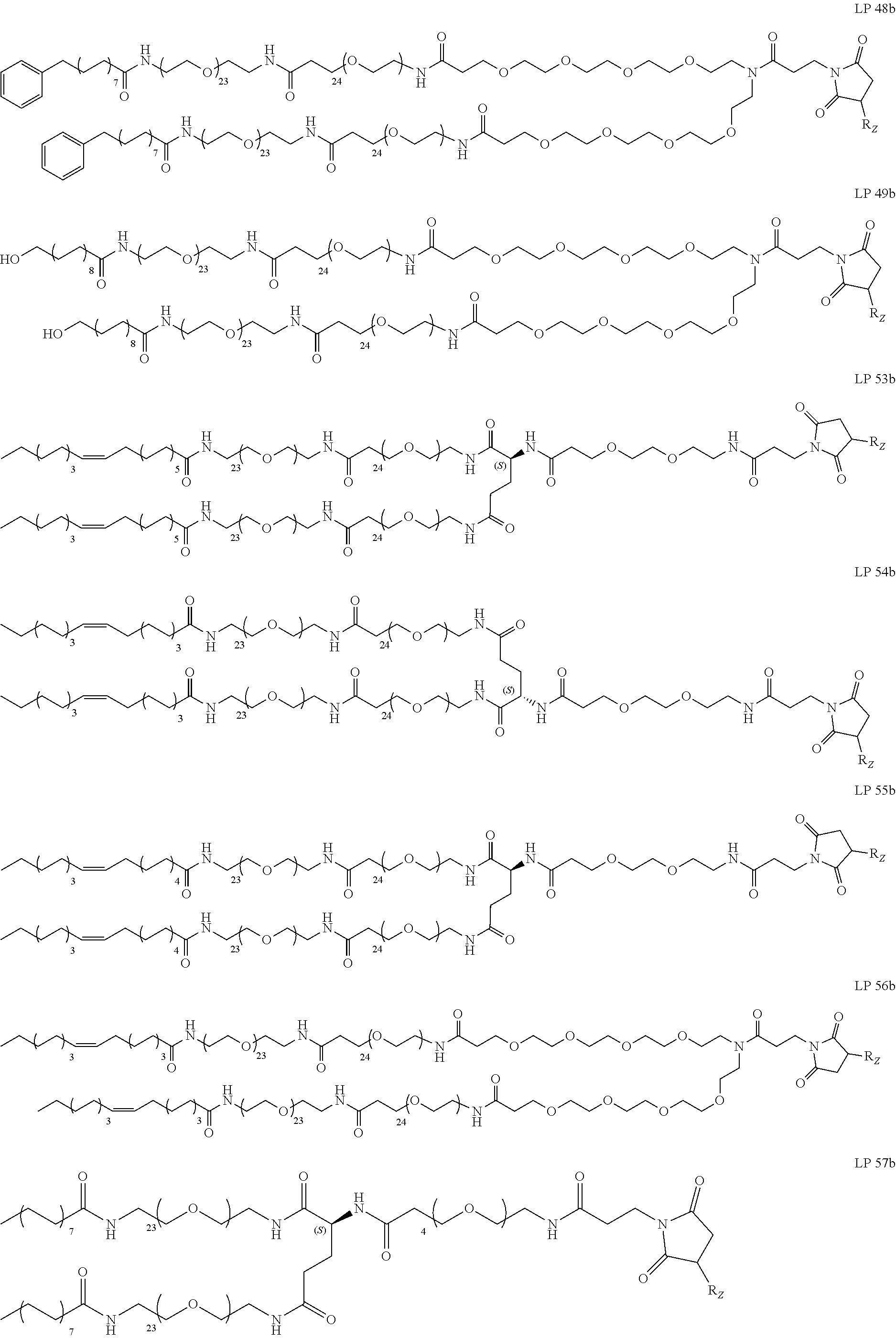 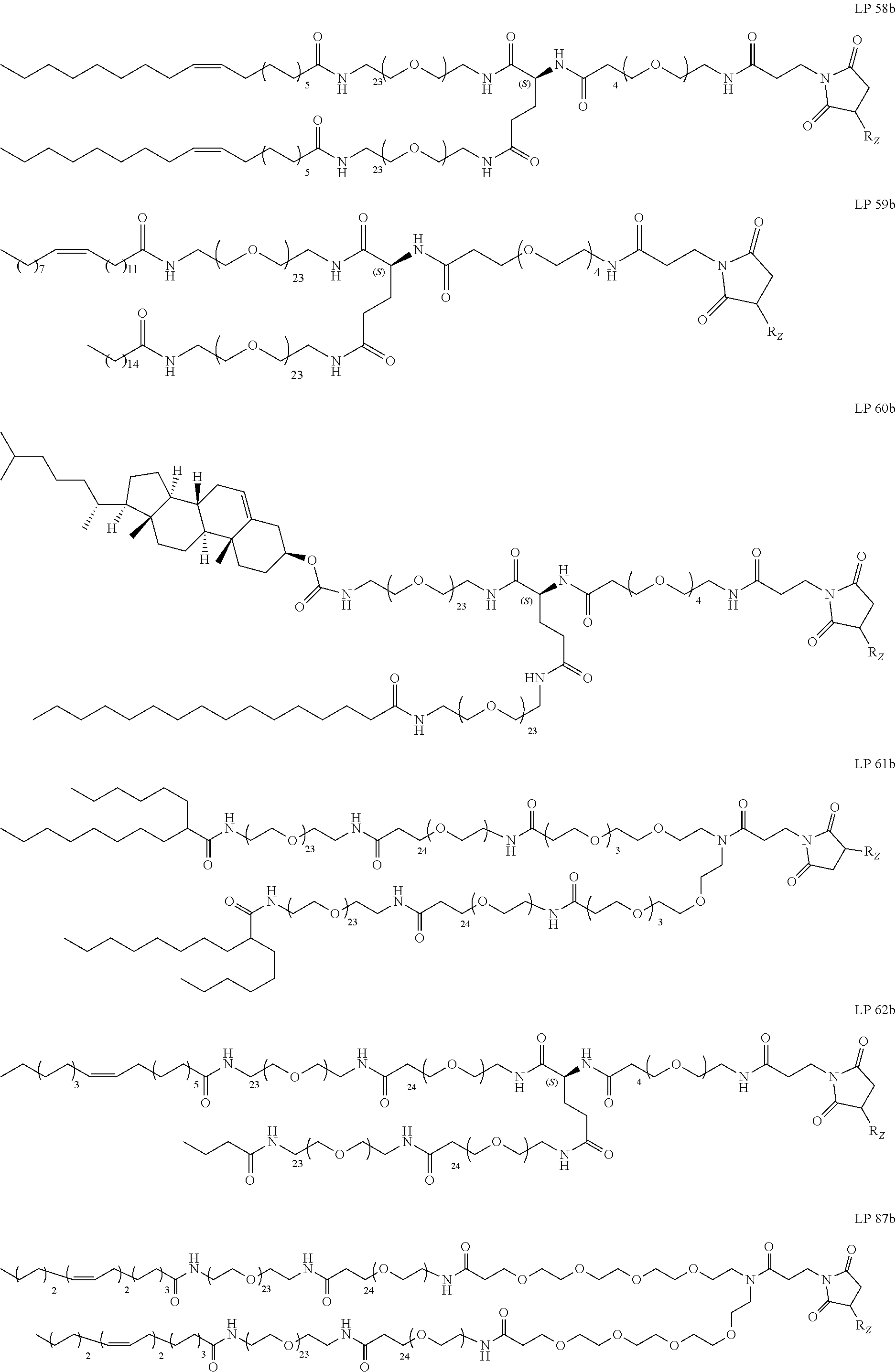 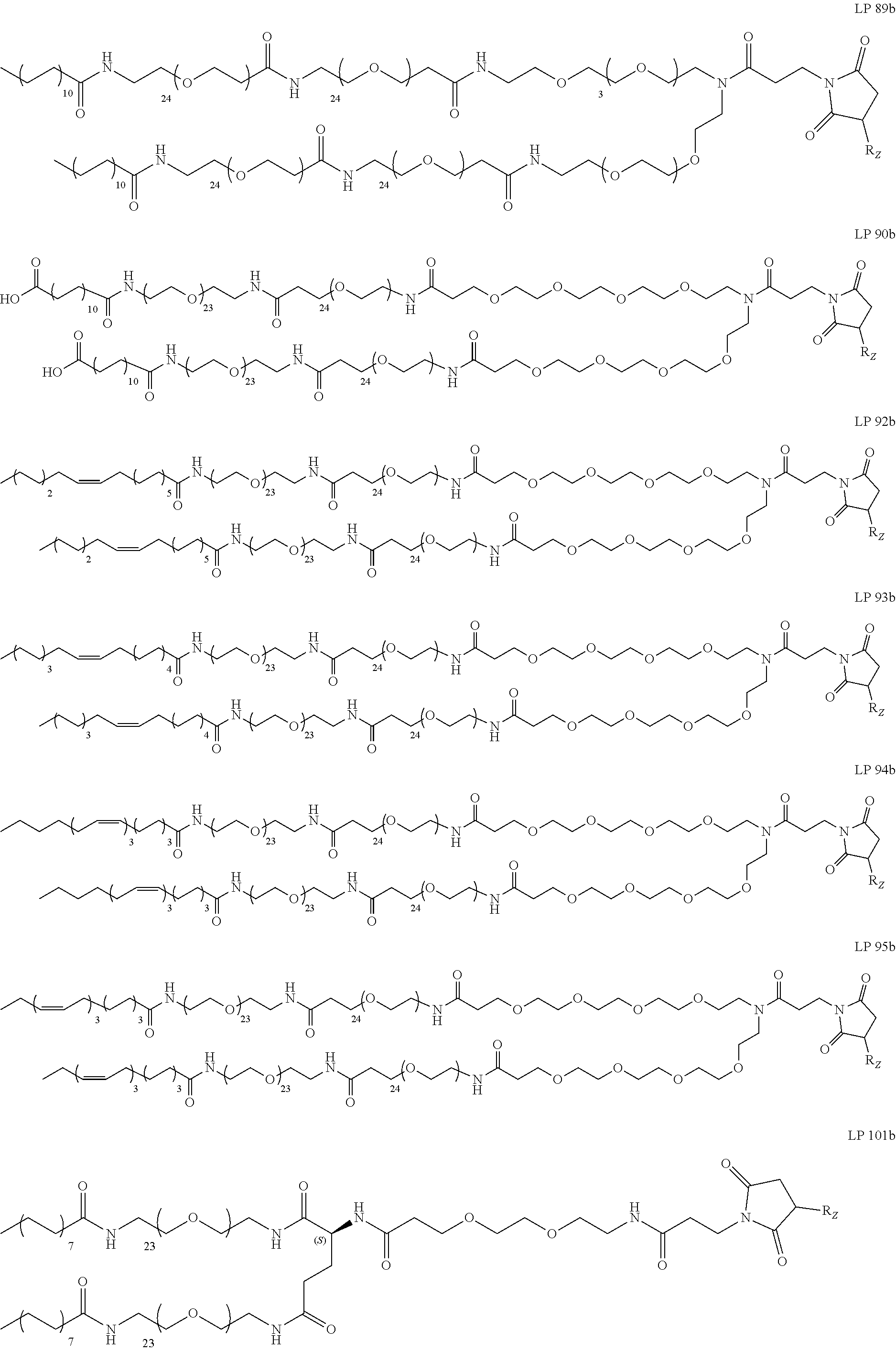 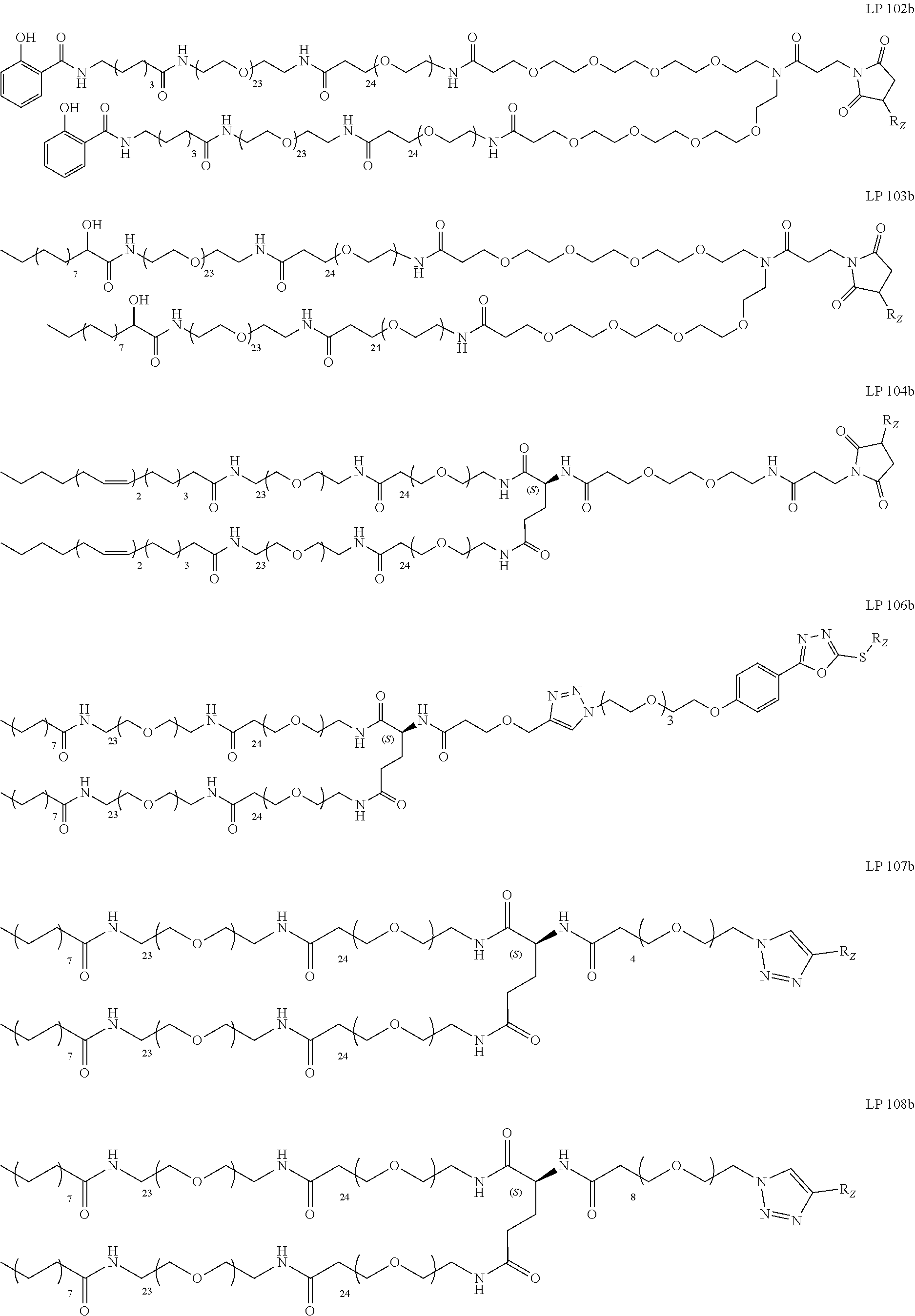 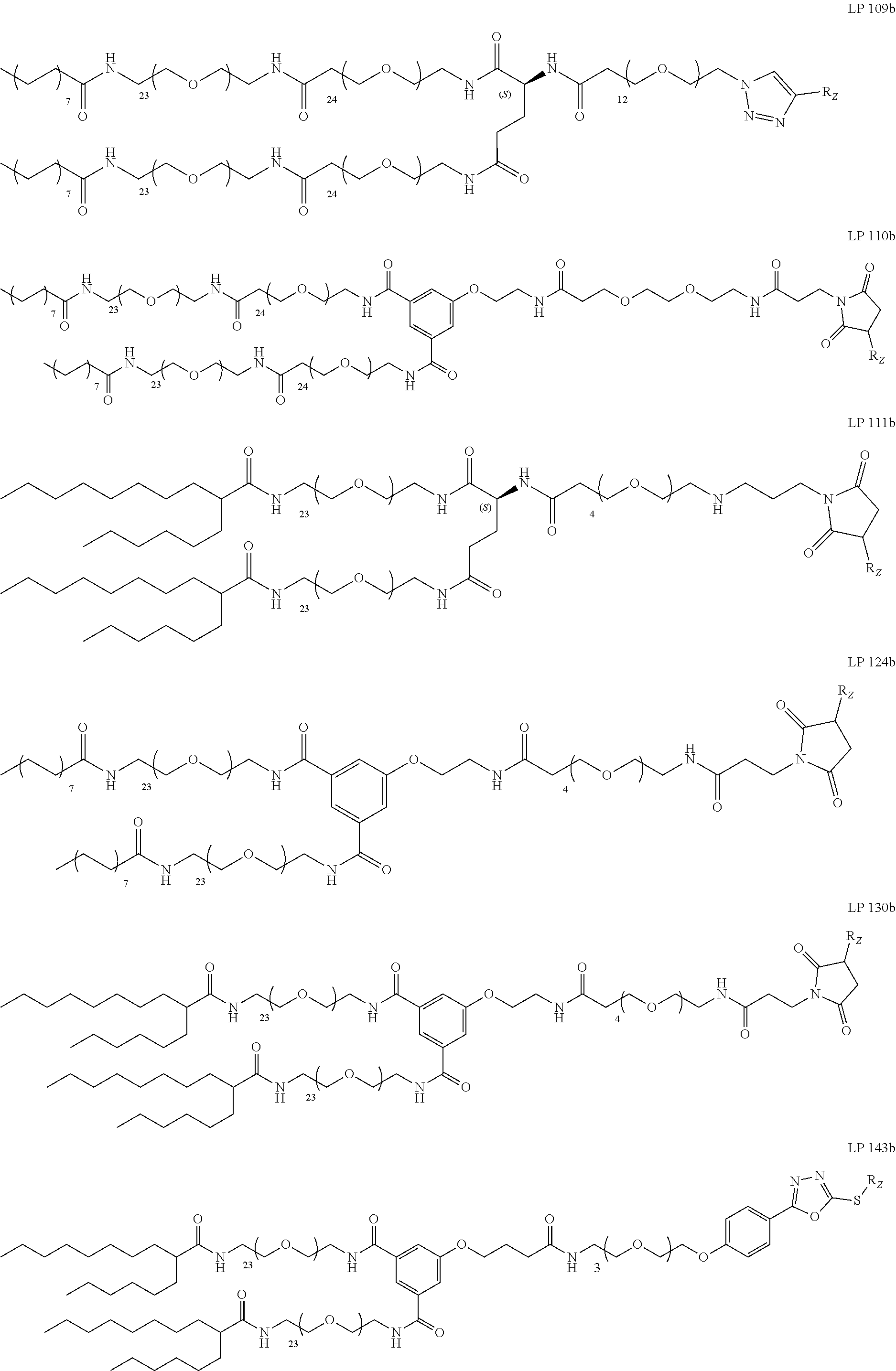 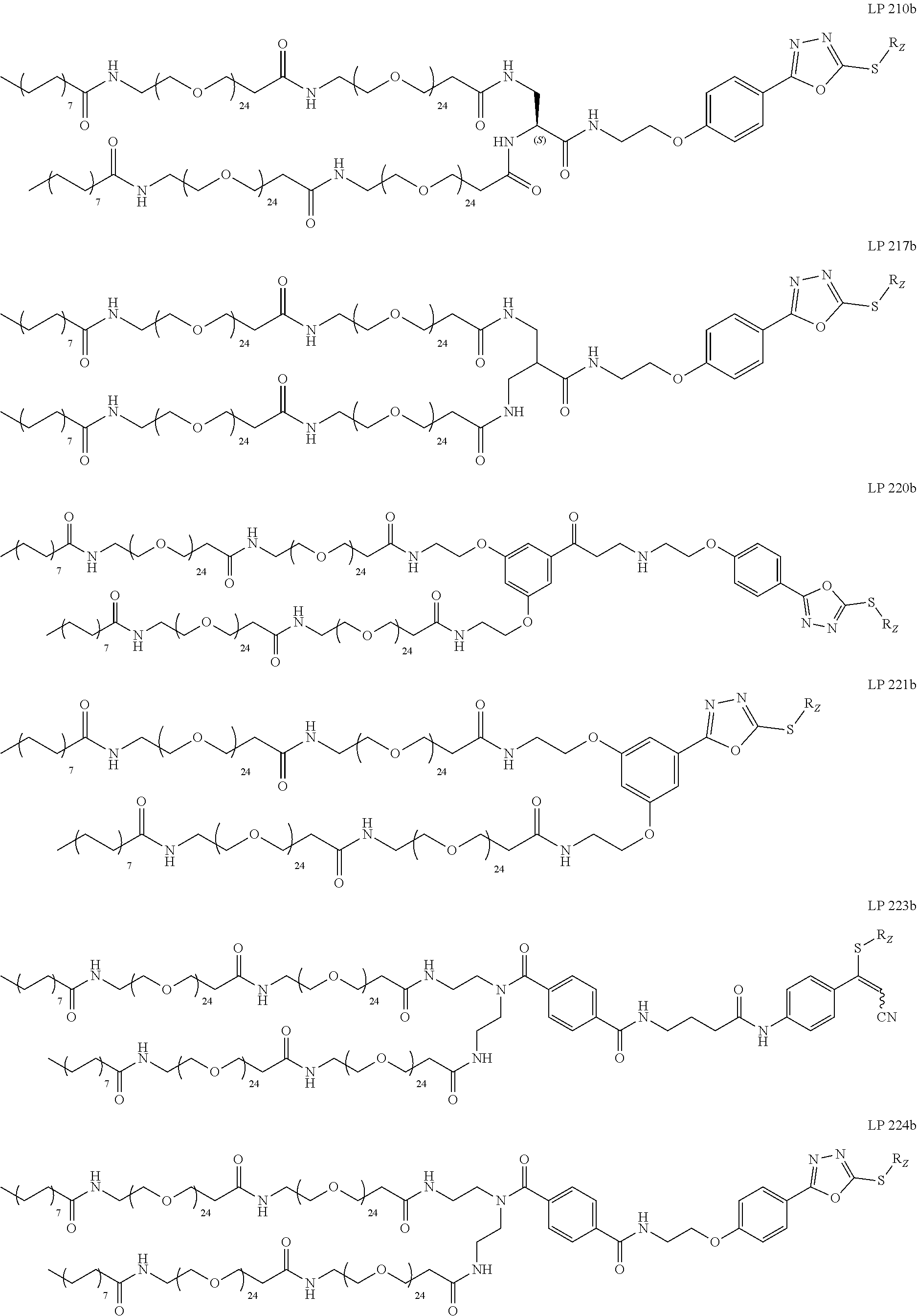 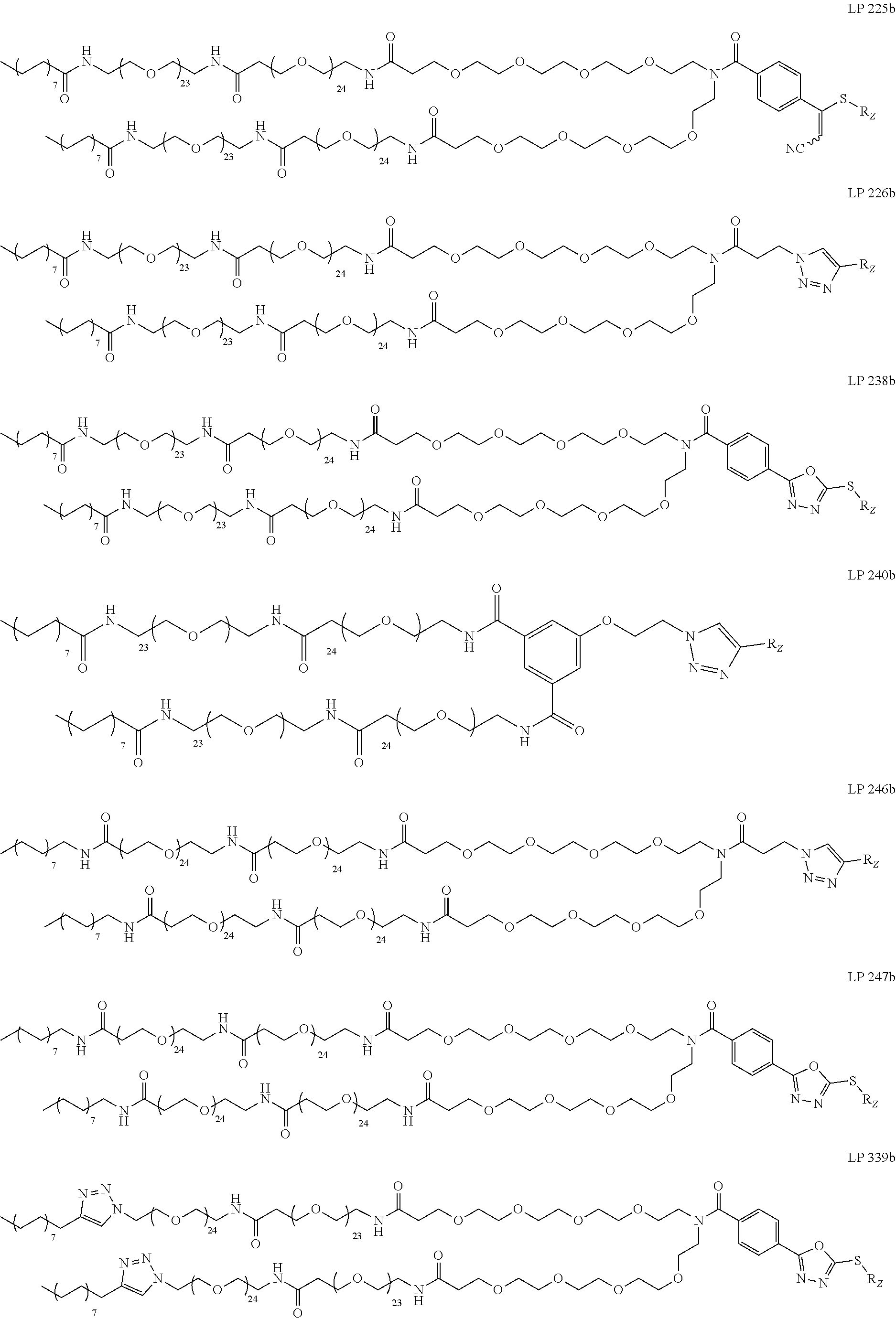 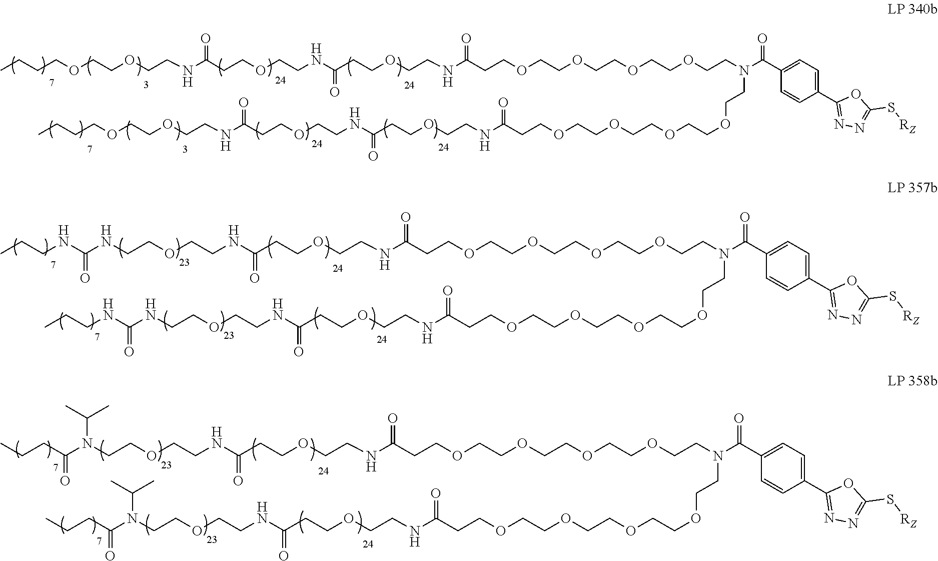
or a pharmaceutically acceptable salt of any of these compounds, wherein each R_{Z} comprises an oligonucleotide-based agent.

27. The compound or pharmaceutically acceptable salt of any one of claims 1-26, wherein the oligonucleotide-based agent is an RNAi agent.

28. A compound of Formula (II): 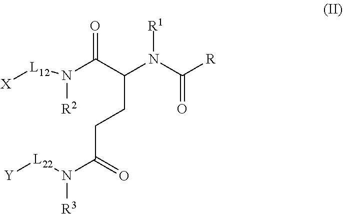
or a pharmaceutically acceptable salt thereof, wherein
R is L_{A2}-R_{Z};
L_{A2} is a bond or a bivalent moiety connecting R_{Z} to —C(O)—;
R_{Z} comprises an oligonucleotide-based agent;
R¹, R² and R₃ are each independently hydrogen or C₁₋₆ alkyl;
L₁₂ and L₂₂ are each independently linkers comprising at least about 5 PEG units; and
X and Y are each independently lipids comprising from about 10 to about 50 carbon atoms.

29. The compound or pharmaceutically acceptable salt of claim 28, wherein L₁₂ and L₂₂ each independently comprise about 15 to about 100 PEG units.

30. The compound or pharmaceutically acceptable salt of claim 28 or claim 29, wherein L₁₂ and L₂₂ each independently comprise about 20 to about 60 PEG units.

31. The compound or pharmaceutically acceptable salt of any one of claims 28-30, wherein L₁₂ and L₂₂ each independently comprise about 20 to about 30 PEG units.

32. The compound or pharmaceutically acceptable salt of any one of claims 28-30, wherein L₁₂ and L₂₂ each comprise about 40 to about 60 PEG units.

33. The compound or pharmaceutically acceptable salt of claim 28, wherein one of L₁₂ and L₂₂ comprises about 20 to about 30 PEG units and the other comprises about 40 to about 60 PEG units.

34. The compound or pharmaceutically acceptable salt of claim 28, wherein each of L₁₂ and L₂₂ is independently selected from the group consisting of:

| Name | Structure |
|---|---|
| Linker 1-2 | 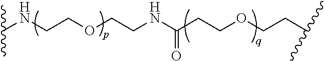 |
| Linker 2-2 | 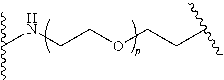 |

wherein
p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
each [symbol] indicates a point of connection to X, Y, —NR²—, or —NR³—, provided that
(i) in Linker 1-2, p+q≥5, and
(ii) in Linker 2-2, p≥5.

35. The compound or pharmaceutically acceptable salt of claim 34, wherein
each p is independently 20, 21, 22, 23, 24, or 25; and
each q is 20, 21, 22, 23, 24, or 25.

36. The compound or pharmaceutically acceptable salt of any one of claims 28-35, wherein at least one of X and Y is an unsaturated lipid.

37. The compound or pharmaceutically acceptable salt of any one of claims 28-36, wherein at least one of X and Y is a saturated lipid.

38. The compound or pharmaceutically acceptable salt of any one of claims 28-37, wherein at least one of X and Y is a branched lipid.

39. The compound or pharmaceutically acceptable salt of any one of claims 28-38, wherein at least one of X and Y is a straight chain lipid.

40. The compound or pharmaceutically acceptable salt of any one of claims 28-39, wherein at least one of X and Y is a lipid comprising from about 10 to about 25 carbon atoms.

41. The compound or pharmaceutically acceptable salt of any one of claims 28-40, wherein at least one of X and Y is cholesteryl.

42. The compound or pharmaceutically acceptable salt of any one of claims 28-35, wherein at least one of X and Y is selected from the group consisting of:

| Name | Structure |
|---|---|
| Lipid 1 | 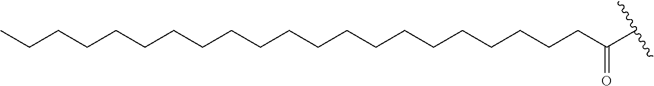 [SMILES: *C(=O)CCCCCCCCCCCCCCCCCCCCC] |
| Lipid 2 | 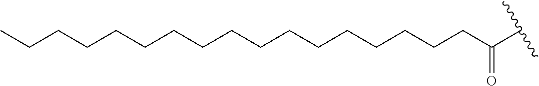 [SMILES: *C(=O)CCCCCCCCCCCCCCCCC] |
| Lipid 3 | 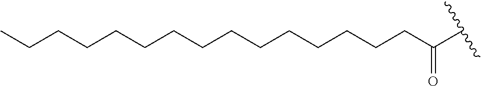 [SMILES: *C(=O)CCCCCCCCCCCCCCC] |
| Lipid 4 | 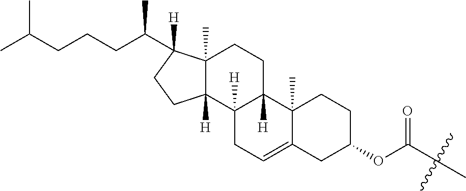 |
| Lipid 5 | 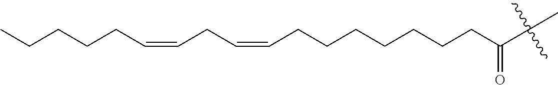 [SMILES: *C(=O)CCCCCCC/C=C\C/C=C\CCCCC] |
| Lipid 6 | 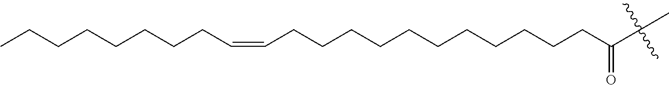 [SMILES: *C(=O)CCCCCCCCCCC/C=C\CCCCCCCC] |
| Lipid 7 | 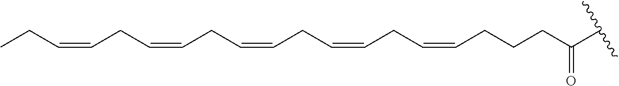 [SMILES: *C(=O)CCC/C=C\C/C=C\C/C=C\C/C=C\C/C=C\CC] |
| Lipid 8 | 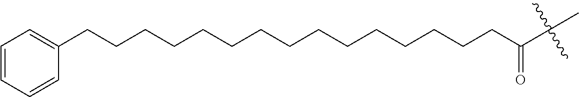 [SMILES: *C(=O)CCCCCCCCCCCCCCCc1ccccc1] |
| Lipid 9 | 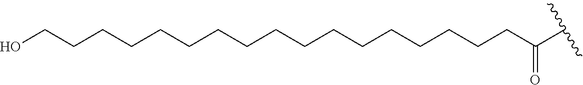 [SMILES: *C(=O)CCCCCCCCCCCCCCCCCO] |
| Lipid 10 | 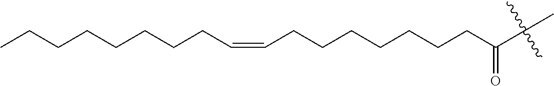 [SMILES: *C(=O)CCCCCCC/C=C\CCCCCCCC] |
| Lipid 11 | 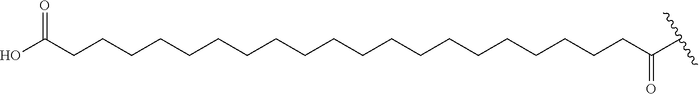 [SMILES: *C(=O)CCCCCCCCCCCCCCCCCCCCC(=O)O] |
| Lipid 12 | 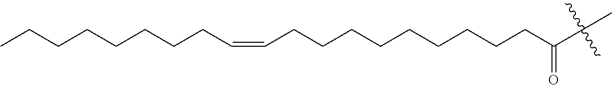 [SMILES: *C(=O)CCCCCCCCC/C=C\CCCCCCCC] |
| Lipid 14 | 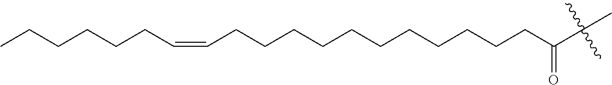 [SMILES: *C(=O)CCCCCCCCCCC/C=C\CCCCCC] |
| Lipid 15 | 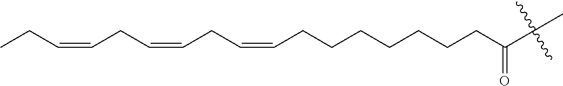 [SMILES: *C(=O)CCCCCCC/C=C\C/C=C\C/C=C\CC] |
| Lipid 16 | 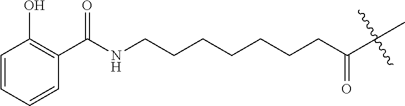 [SMILES: *C(=O)CCCCCCCNC(=O)c1ccccc1O] |
| Lipid 17 | 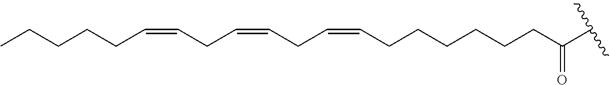 [SMILES: *C(=O)CCCCCC/C=C\C/C=C\C/C=C\CCCCC] |
| Lipid 18 | 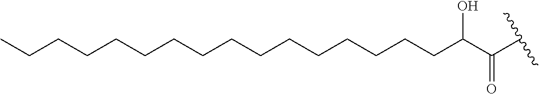 [SMILES: *C(=O)C(O)CCCCCCCCCCCCCCCC] |
| Lipid 19 | 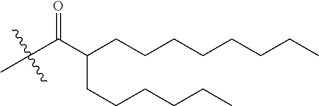 [SMILES: *C(=O)C(CCCCCC)CCCCCCCC] |
| Lipid 20 | 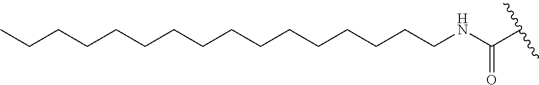 [SMILES: *C(=O)NCCCCCCCCCCCCCCCC] |
| Lipid 21 | 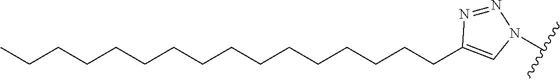 [SMILES: *n1cc(CCCCCCCCCCCCCCCC)nn1] |
| Lipid 22 | 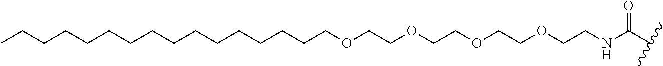 [SMILES: *C(=O)NCCOCCOCCOCCOCCCCCCCCCCCCCCCC] |
| Lipid 23 | 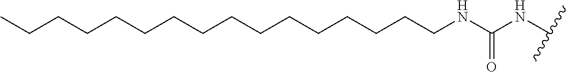 [SMILES: *NC(=O)NCCCCCCCCCCCCCCCC] |
| Lipid 24 | 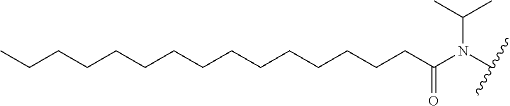 [SMILES: *N(C(=O)CCCCCCCCCCCCCCC)C(C)C] |

wherein [symbol] indicates a point of connection to L₁₂ or L₂₂.

43. The compound or pharmaceutically acceptable salt of any one of claims 28-35, wherein each of X and Y are independently selected from the group consisting of:

| Name | Structure |
|---|---|
| Lipid 1 | 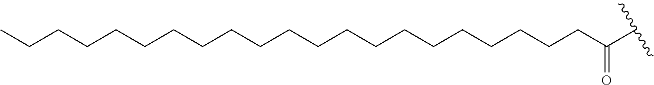 [SMILES: *C(=O)CCCCCCCCCCCCCCCCCCCCC] |
| Lipid 2 | 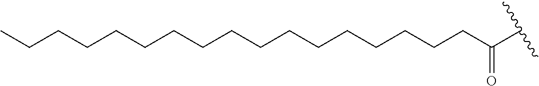 [SMILES: *C(=O)CCCCCCCCCCCCCCCCC] |
| Lipid 3 | 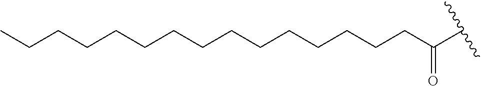 [SMILES: *C(=O)CCCCCCCCCCCCCCC] |
| Lipid 4 | 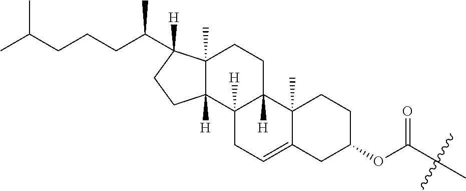 |
| Lipid 5 | 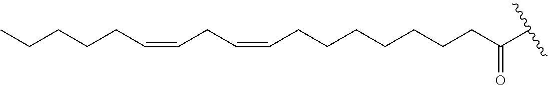 [SMILES: *C(=O)CCCCCCC/C=C\C/C=C\CCCCC] |
| Lipid 6 | 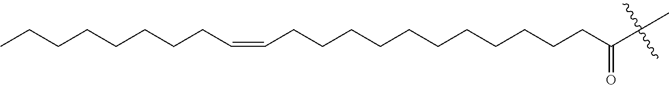 [SMILES: *C(=O)CCCCCCCCCCC/C=C\CCCCCCCC] |
| Lipid 7 | 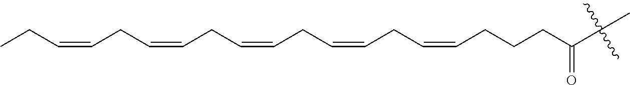 [SMILES: *C(=O)CCC/C=C\C/C=C\C/C=C\C/C=C\C/C=C\CC] |
| Lipid 8 | 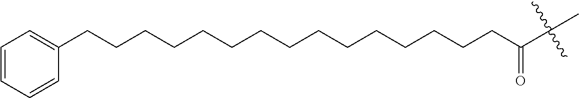 [SMILES: *C(=O)CCCCCCCCCCCCCCCc1ccccc1] |
| Lipid 9 | 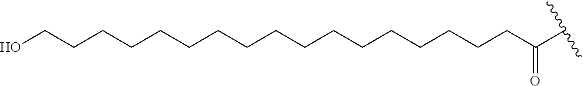 [SMILES: *C(=O)CCCCCCCCCCCCCCCCCO] |
| Lipid 10 | 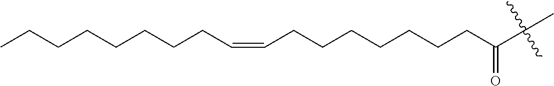 [SMILES: *C(=O)CCCCCCC/C=C\CCCCCCCC] |
| Lipid 11 | 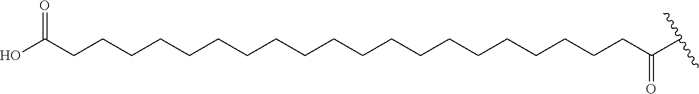 [SMILES: *C(=O)CCCCCCCCCCCCCCCCCCCCC(=O)O] |
| Lipid 12 | 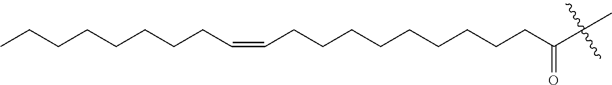 [SMILES: *C(=O)CCCCCCCCC/C=C\CCCCCCCC] |
| Lipid 14 | 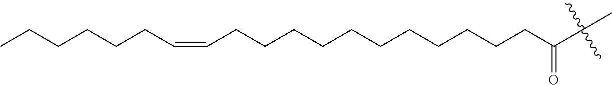 [SMILES: *C(=O)CCCCCCCCCCC/C=C\CCCCCC] |
| Lipid 15 | 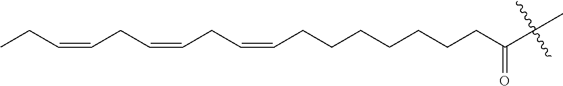 [SMILES: *C(=O)CCCCCCC/C=C\C/C=C\C/C=C\CC] |
| Lipid 16 | 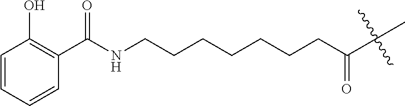 [SMILES: *C(=O)CCCCCCCNC(=O)c1ccccc1O] |
| Lipid 17 | 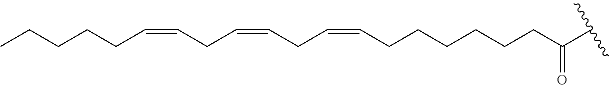 [SMILES: *C(=O)CCCCCC/C=C\C/C=C\C/C=C\CCCCC] |
| Lipid 18 | 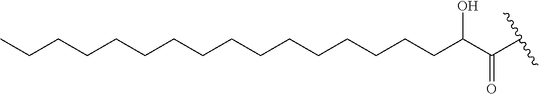 [SMILES: *C(=O)C(O)CCCCCCCCCCCCCCCC] |
| Lipid 19 | 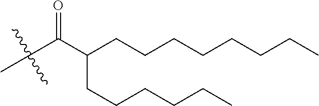 [SMILES: *C(=O)C(CCCCCC)CCCCCCCC] |
| Lipid 20 | 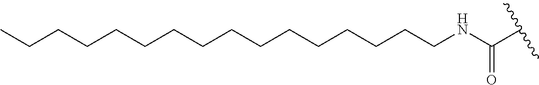 [SMILES: *C(=O)NCCCCCCCCCCCCCCCC] |
| Lipid 21 | 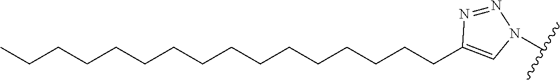 [SMILES: *n1cc(CCCCCCCCCCCCCCCC)nn1] |
| Lipid 22 | 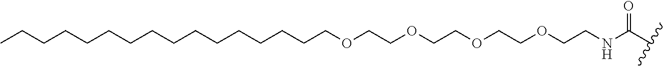 [SMILES: *C(=O)NCCOCCOCCOCCOCCCCCCCCCCCCCCCC] |
| Lipid 23 | 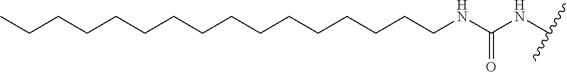 [SMILES: *NC(=O)NCCCCCCCCCCCCCCCC] |
| Lipid 24 | 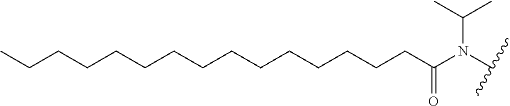 [SMILES: *N(C(=O)CCCCCCCCCCCCCCC)C(C)C] |

wherein [symbol] indicates a point of connection to L₁₂ or L₂₂.

44. The compound or pharmaceutically acceptable salt of claim 43, wherein each of X and Y are independently selected from the group consisting of:

| Name | Structure |
|---|---|
| Lipid 3 | 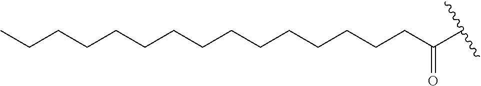 [SMILES: *C(=O)CCCCCCCCCCCCCCC] |
| Lipid 4 | 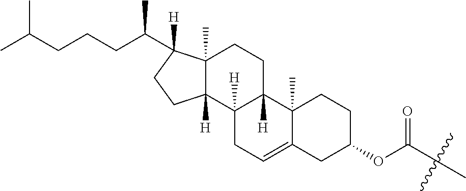 |
| Lipid 5 | 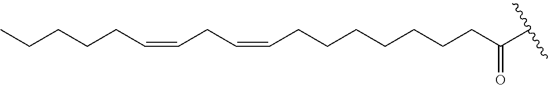 [SMILES: *C(=O)CCCCCCC/C=C\C/C=C\CCCCC] |
| Lipid 6 | 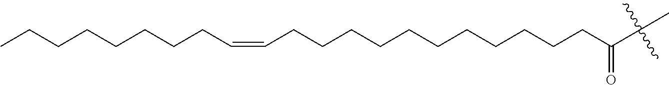 [SMILES: *C(=O)CCCCCCCCCCC/C=C\CCCCCCCC] |
| Lipid 7 | 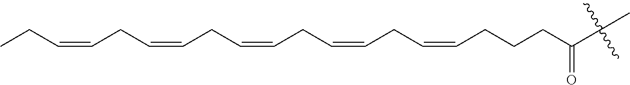 [SMILES: *C(=O)CCC/C=C\C/C=C\C/C=C\C/C=C\C/C=C\CC] |
| Lipid 10 | 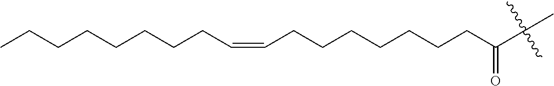 [SMILES: *C(=O)CCCCCCC/C=C\CCCCCCCC] |
| Lipid 12 | 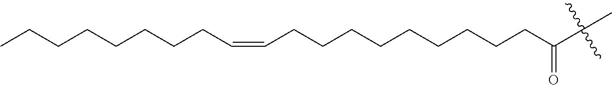 [SMILES: *C(=O)CCCCCCCCC/C=C\CCCCCCCC] |
| Lipid 19 | 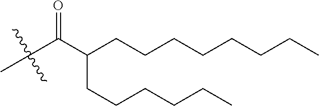 [SMILES: *C(=O)C(CCCCCC)CCCCCCCC] |

wherein [symbol] indicates a point of connection to L₁₂ or L₂₂.

45. The compound or pharmaceutically acceptable salt of any one of claims 28-44, wherein L_{A2} is selected from the group consisting of:

| Name | Structure |
|---|---|
| Tether 1-2 | 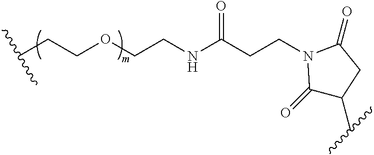 |
| Tether 2-2 | 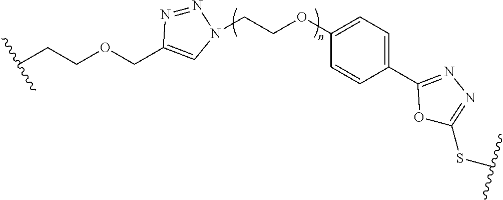 |
| Tether 3-2 | 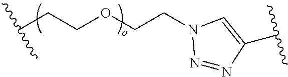 |

wherein each of m, n, and o is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and [symbol] indicates a point of connection to R_{Z} or —C(O)—.

46. The compound or pharmaceutically acceptable salt of claim 45, wherein
m is 1, 2, 3, 4, 5, 6, 7, 8, 9, 10, 20, 21, 22, 23, or 25;
n is 2, 3, 4, or 5; and
o is 2, 3, 4, 5, 6, 7, 8, 9, 10, 11, 12, or 13.

47. The compound or pharmaceutically acceptable salt of any one of claims 28-46, wherein each of R¹, R² and R³ is independently hydrogen or C₁₋₃ alkyl.

48. The compound or pharmaceutically acceptable salt of any one of claims 28-47, wherein each of R¹, R² and R³ is hydrogen.

49. The compound or pharmaceutically acceptable salt of claim 28, wherein the compound of Formula (II) is selected from the group consisting of: 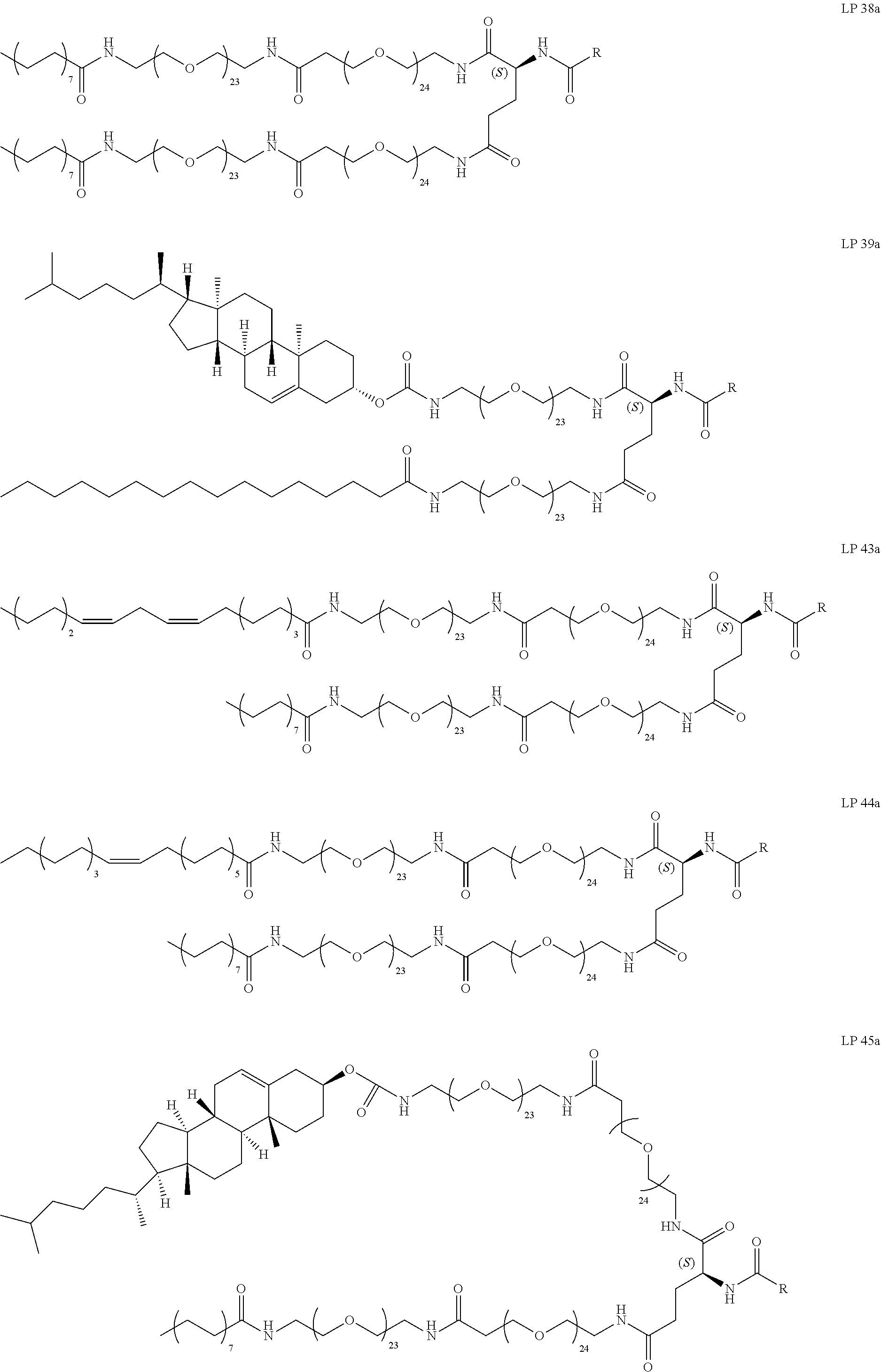 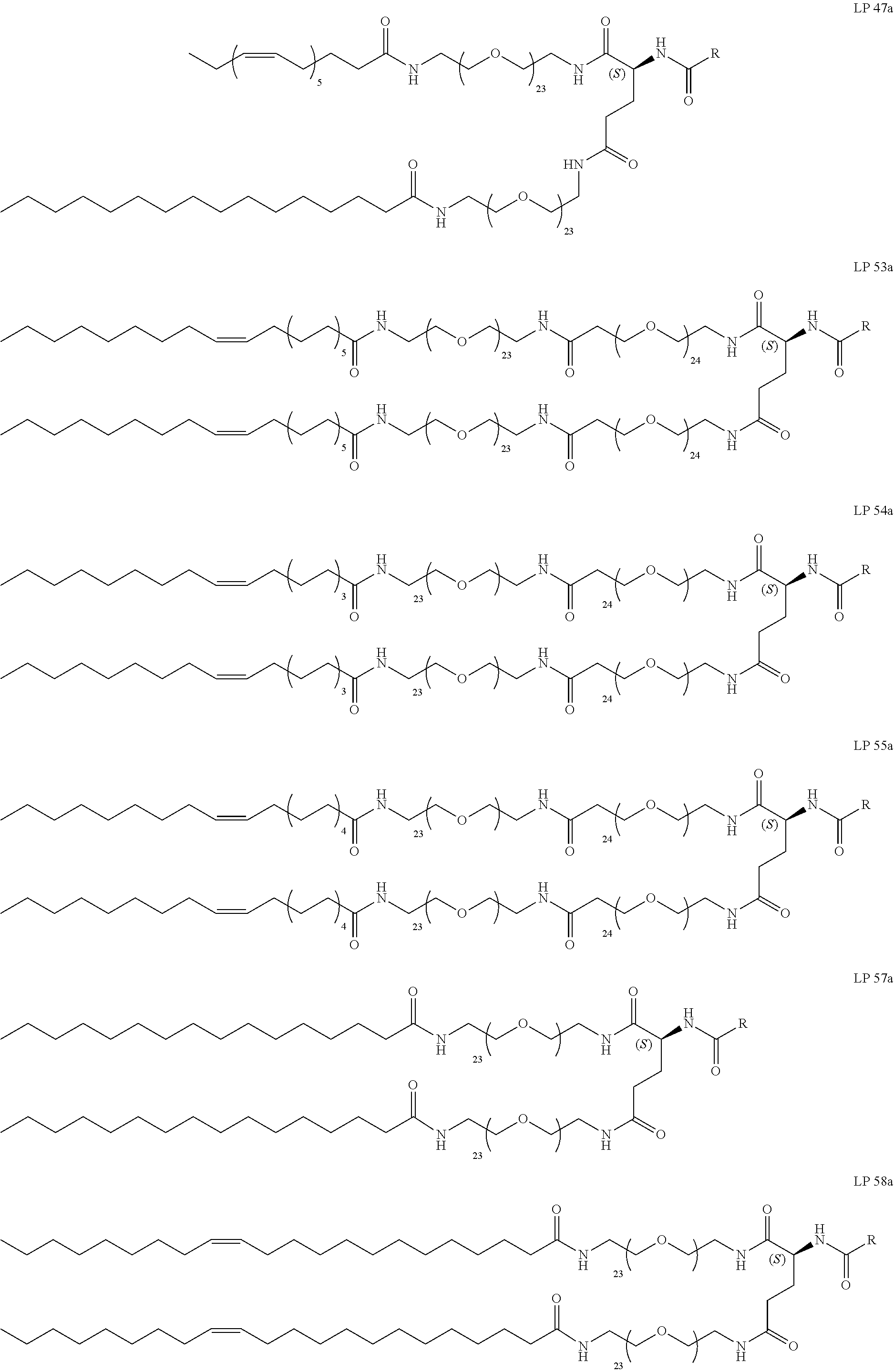 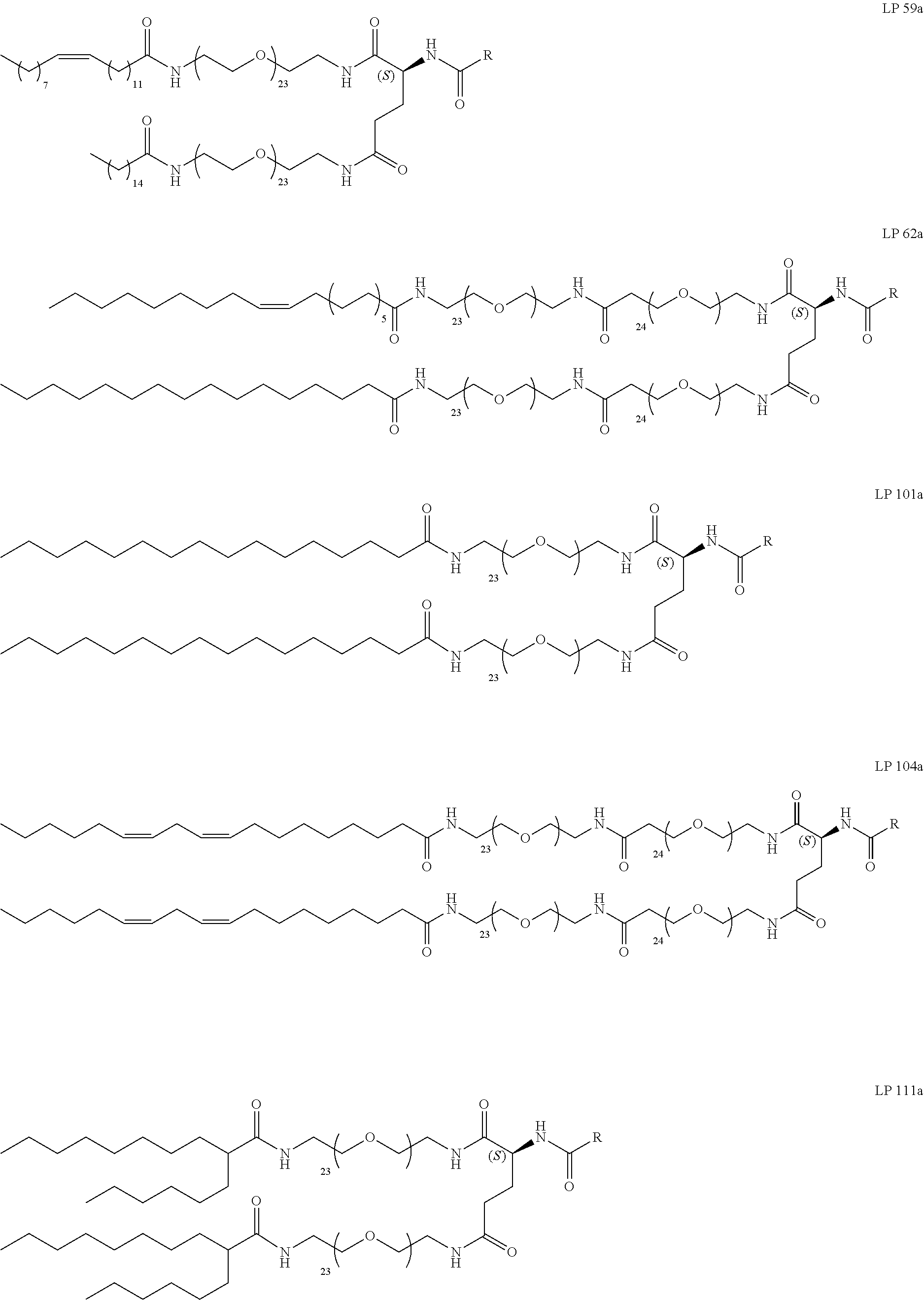
or a pharmaceutically acceptable salt of any of these compounds.

50. The compound or pharmaceutically acceptable salt of claim 28, wherein the compound of Formula (II) selected from the group consisting of: 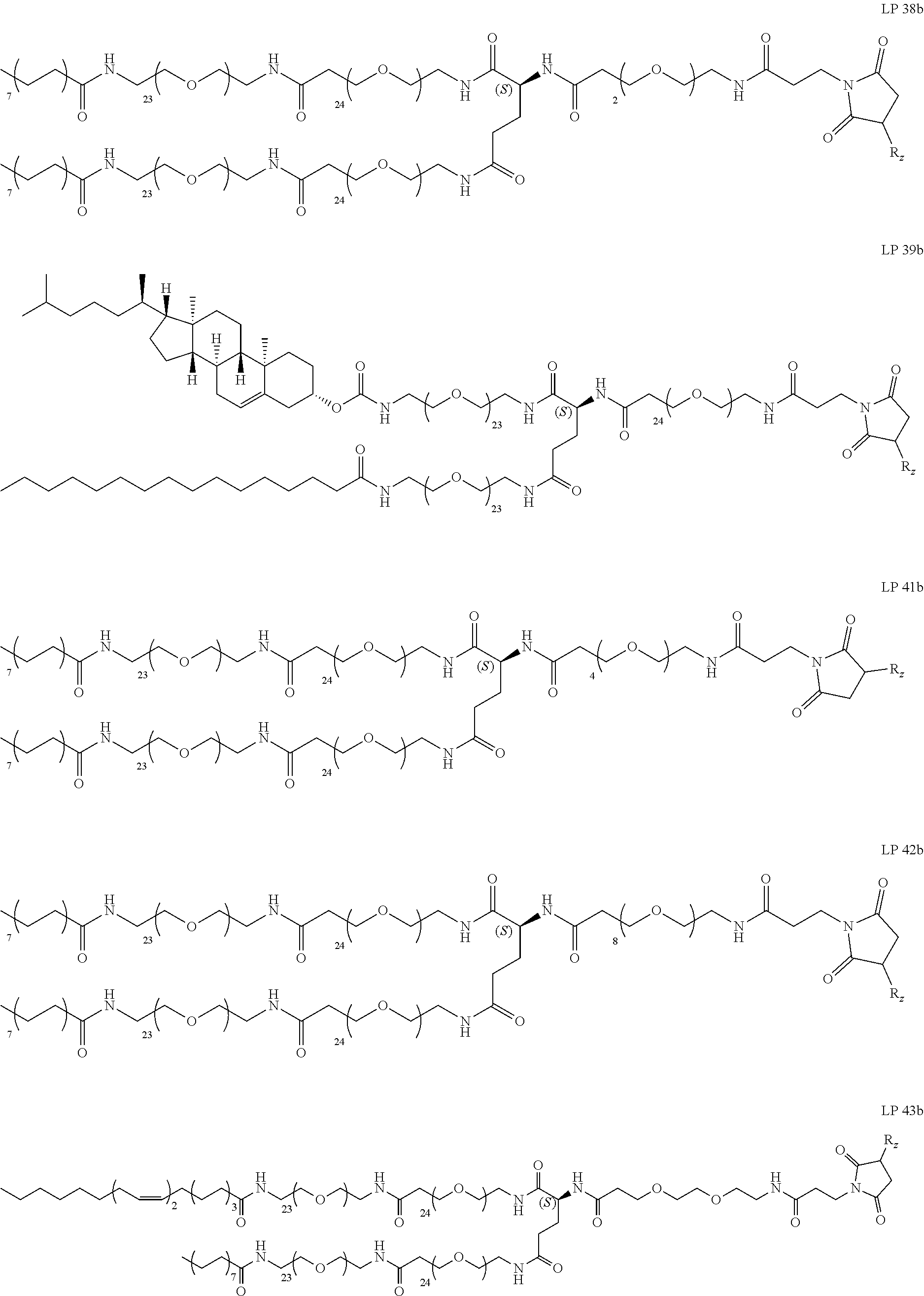 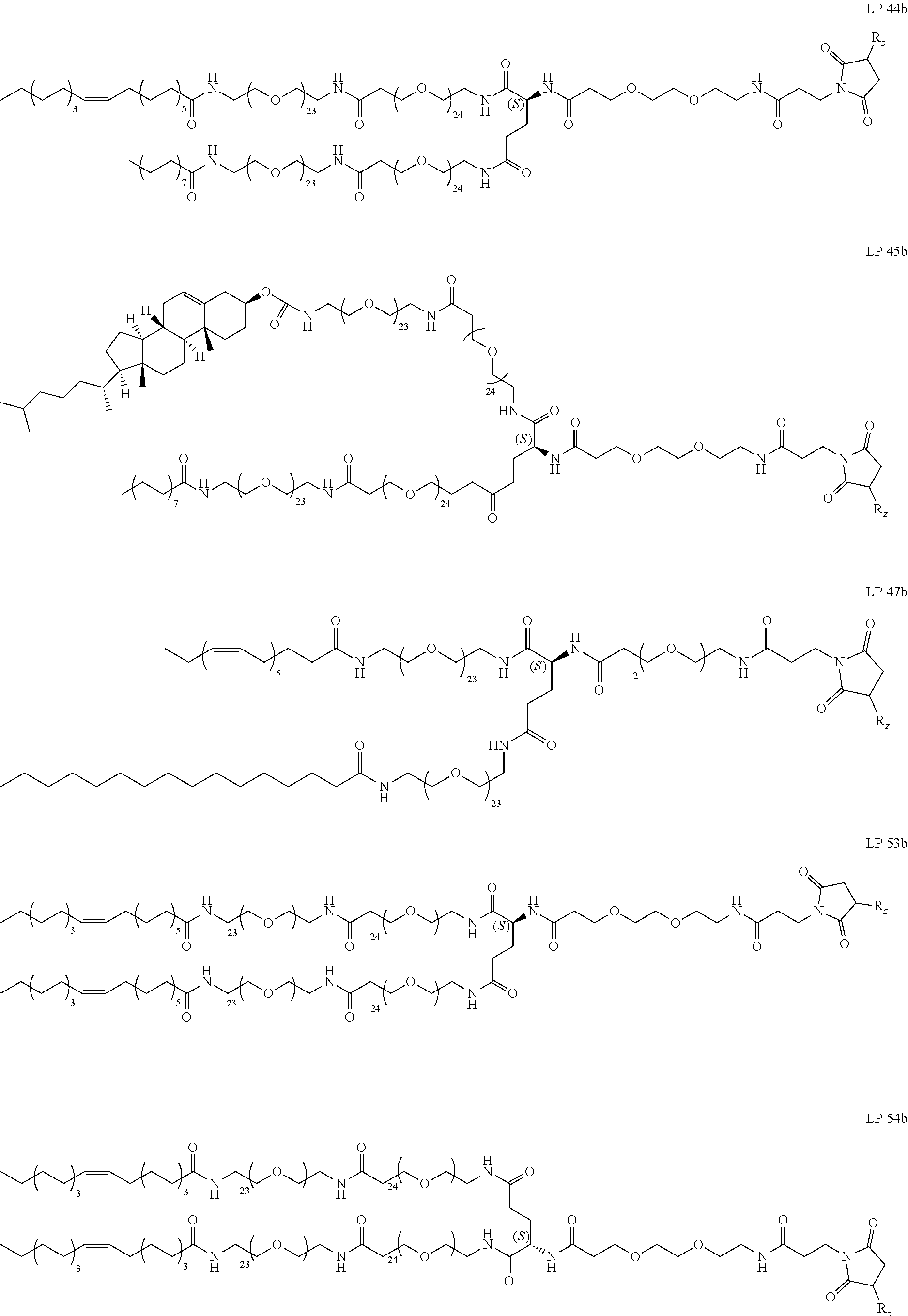 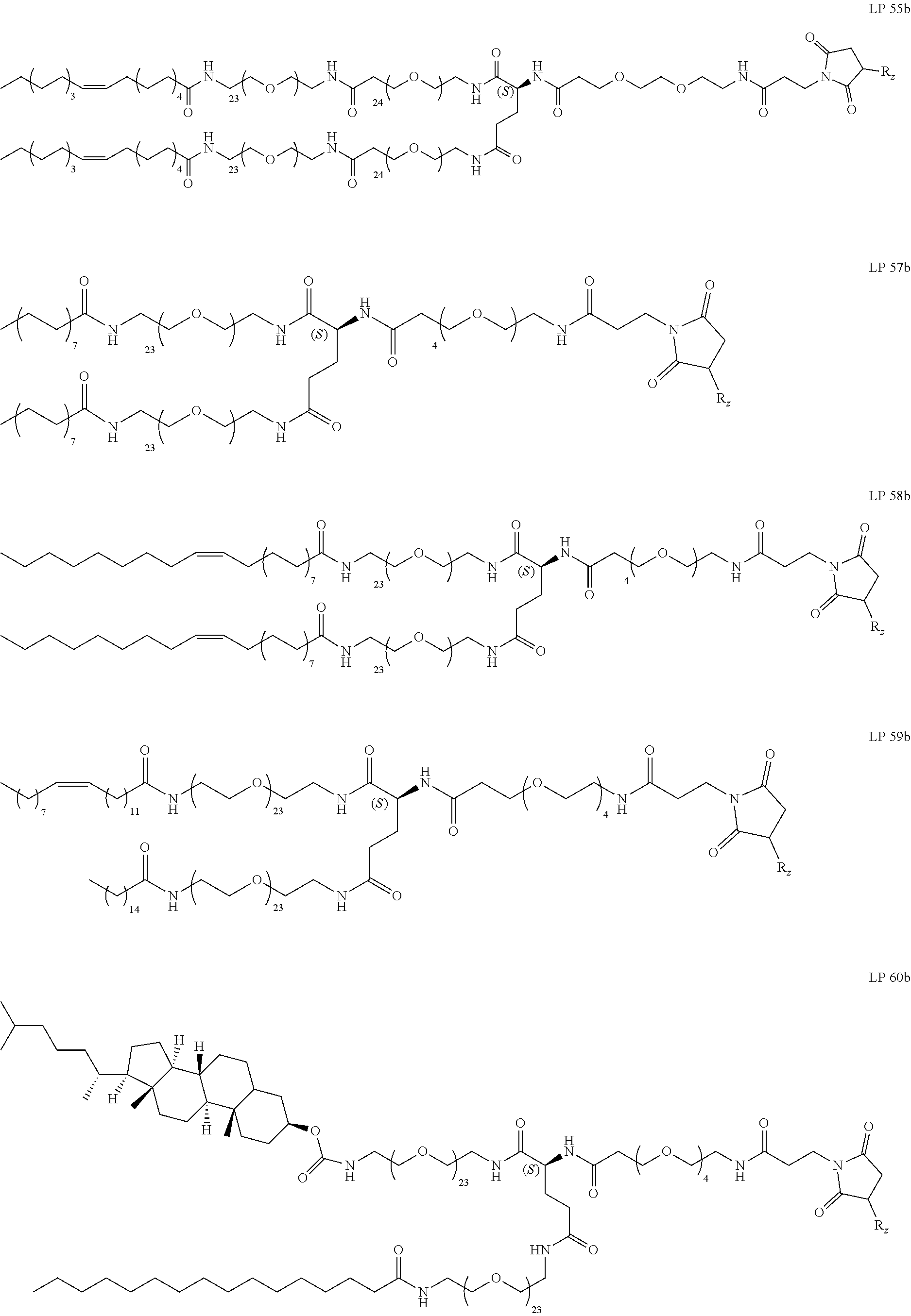 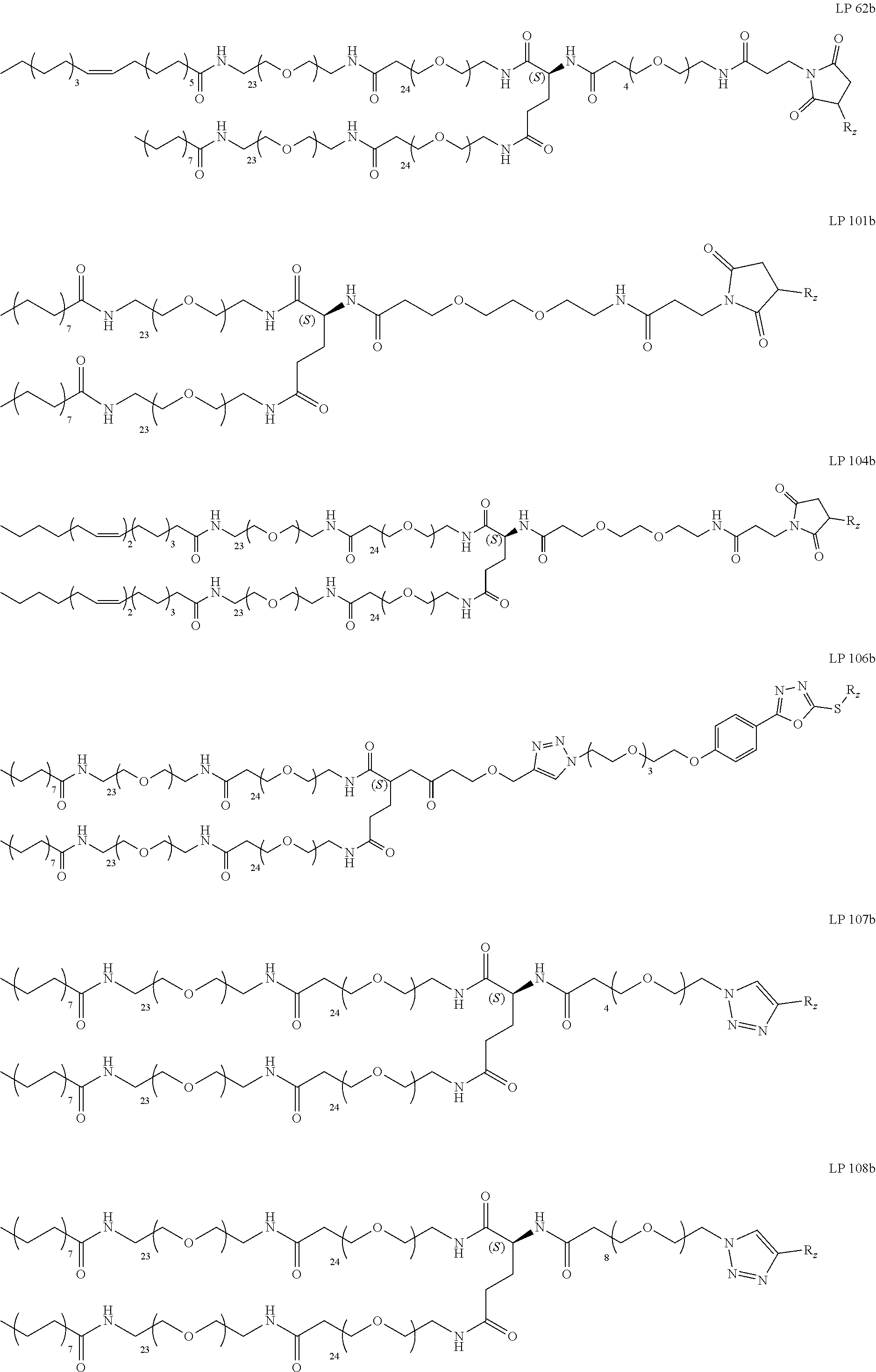 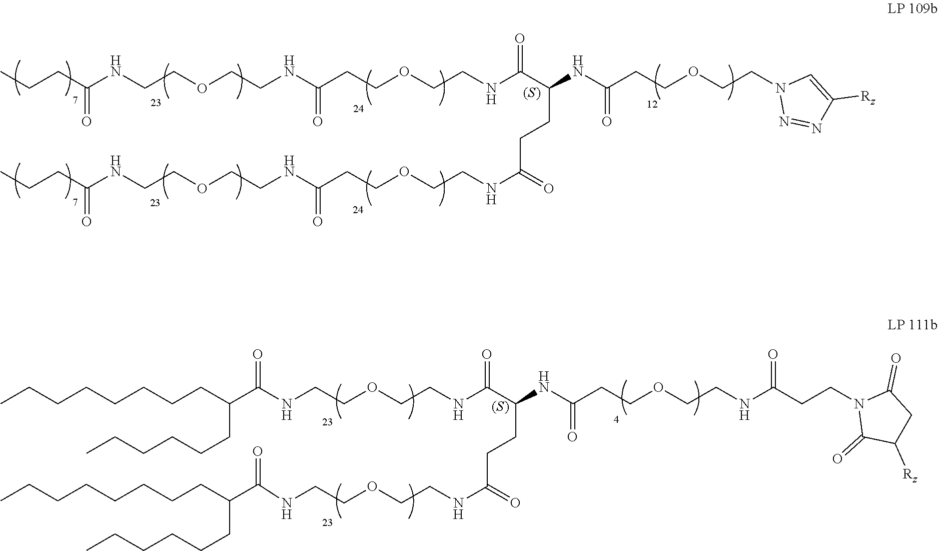
or a pharmaceutically acceptable salt of any of these compounds.

51. The compound or pharmaceutically acceptable salt of any one of claims 28-50, wherein the oligonucleotide-based agent is an RNAi agent.

52. A compound of Formula (III): 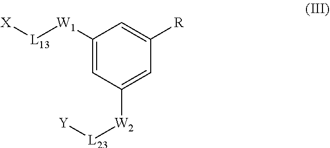
or a pharmaceutically acceptable salt thereof, wherein
R is L_{A3}-R_{Z};
L_{A3} is a bond or a bivalent moiety connecting R_{Z} to the phenyl ring;
R_{Z} comprises an oligonucleotide-based agent;
W₁ is —C(O)NR₁— or —OCH₂CH₂NR₁C(O)—, wherein R₁ is hydrogen or C₁₋₆ alkyl;
W₂ is —C(O)NR₂— or —OCH₂CH₂NR₂C(O)—, wherein R₂ is hydrogen or C₁₋₆ alkyl;
L₁₃ and L₂₃ are each independently linkers comprising at least about 5 PEG units; and
X and Y are each independently lipids comprising from about 10 to about 50 carbon atoms.

53. The compound or pharmaceutically acceptable salt of claim 52, wherein L₁₃ and L₂₃ each independently comprise about 15 to about 100 PEG units.

54. The compound or pharmaceutically acceptable salt of claim 52 or claim 53, wherein L₁₃ and L₂₃ each independently comprise about 20 to about 60 PEG units.

55. The compound or pharmaceutically acceptable salt of any one of claims 52-54, wherein L₁₃ and L₂₃ each independently comprise about 20 to about 30 PEG units.

56. The compound or pharmaceutically acceptable salt of any one of claims 52-54, wherein L₁₃ and L₂₃ each comprise about 40 to about 60 PEG units.

57. The compound or pharmaceutically acceptable salt of claim 52, wherein one of L₁₃ and L₂₃ comprises about 20 to about 30 PEG units and the other comprises about 40 to about 60 PEG units.

58. The compound or pharmaceutically acceptable salt of claim 52, wherein each of L₁₃ and L₂₃ is independently selected from the group consisting of:

| Name | Structure |
|---|---|
| Linker 1-3 | 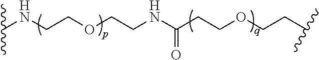 |
| Linker 2-3 | 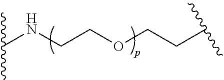 |
| Linker 3-3 | 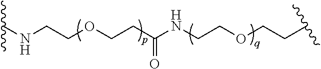 |

wherein
p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
each [symbol] indicates a point of connection to X, Y, W₁ or W₂; provided that
(i) in Linker 1-3 and Linker 3-3, p+q≥5; and
(ii) in Linker 2-3, p≥5.

59. The compound or pharmaceutically acceptable salt of claim 58, wherein
each p is independently 20, 21, 22, 23, 24, or 25; and
each q is independently 20, 21, 22, 23, 24, or 25.

60. The compound or pharmaceutically acceptable salt of any one of claims 52-59, wherein at least one of X and Y is an unsaturated lipid.

61. The compound or pharmaceutically acceptable salt of any one of claims 52-60, wherein at least one of X and Y is a saturated lipid.

62. The compound or pharmaceutically acceptable salt of any one of claims 52-61, wherein at least one of X and Y is a branched lipid.

63. The compound or pharmaceutically acceptable salt of any one of claims 52-62, wherein at least one of X and Y is a straight chain lipid.

64. The compound or pharmaceutically acceptable salt of any one of claims 52-63, wherein at least one of X and Y is a lipid comprising from about 10 to about 25 carbon atoms.

65. The compound or pharmaceutically acceptable salt of any one of claims 52-64, wherein at least one of X and Y is cholesteryl.

66. The compound or pharmaceutically acceptable salt of any one of claims 52-59, wherein at least one of X and Y is selected from the group consisting of:

| Name | Structure |
|---|---|
| Lipid 1 | 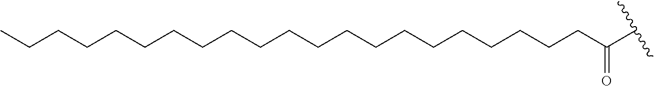 [SMILES: *C(=O)CCCCCCCCCCCCCCCCCCCCC] |
| Lipid 2 | 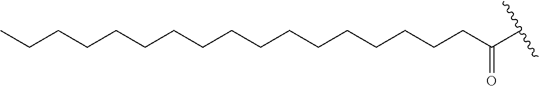 [SMILES: *C(=O)CCCCCCCCCCCCCCCCC] |
| Lipid 3 | 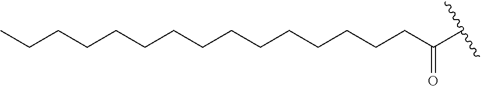 [SMILES: *C(=O)CCCCCCCCCCCCCCC] |
| Lipid 4 | 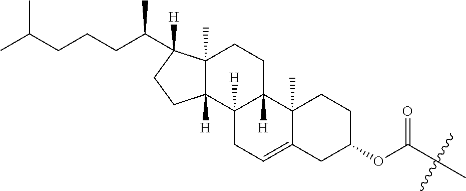 |
| Lipid 5 | 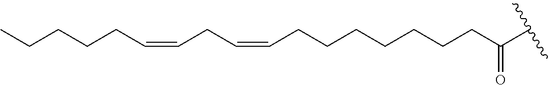 [SMILES: *C(=O)CCCCCCC/C=C\C/C=C\CCCCC] |
| Lipid 6 | 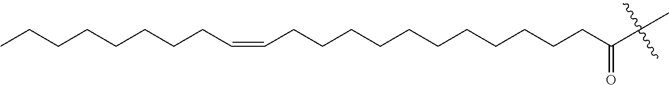 [SMILES: *C(=O)CCCCCCCCCCC/C=C\CCCCCCCC] |
| Lipid 7 | 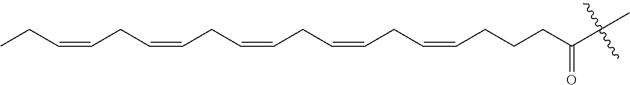 [SMILES: *C(=O)CCC/C=C\C/C=C\C/C=C\C/C=C\C/C=C\CC] |
| Lipid 8 | 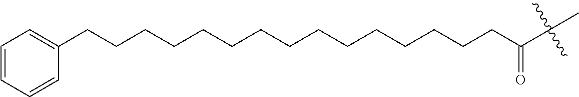 [SMILES: *C(=O)CCCCCCCCCCCCCCCc1ccccc1] |
| Lipid 9 | 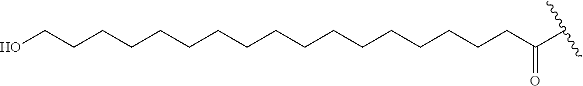 [SMILES: *C(=O)CCCCCCCCCCCCCCCCCO] |
| Lipid 10 | 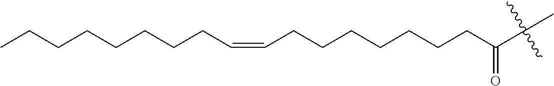 [SMILES: *C(=O)CCCCCCC/C=C\CCCCCCCC] |
| Lipid 11 | 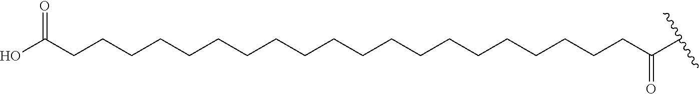 [SMILES: *C(=O)CCCCCCCCCCCCCCCCCCCCC(=O)O] |
| Lipid 12 | 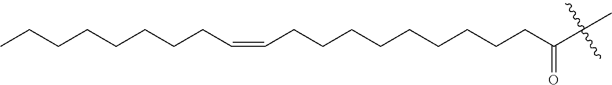 [SMILES: *C(=O)CCCCCCCCC/C=C\CCCCCCCC] |
| Lipid 14 | 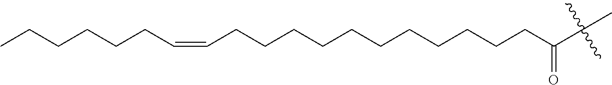 [SMILES: *C(=O)CCCCCCCCCCC/C=C\CCCCCC] |
| Lipid 15 | 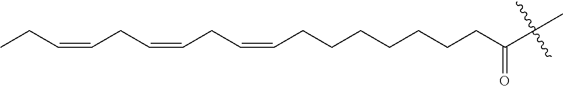 [SMILES: *C(=O)CCCCCCC/C=C\C/C=C\C/C=C\CC] |
| Lipid 16 | 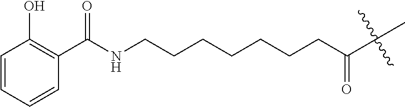 [SMILES: *C(=O)CCCCCCCNC(=O)c1ccccc1O] |
| Lipid 17 | 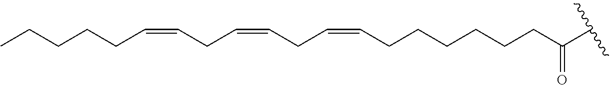 [SMILES: *C(=O)CCCCCC/C=C\C/C=C\C/C=C\CCCCC] |
| Lipid 18 | 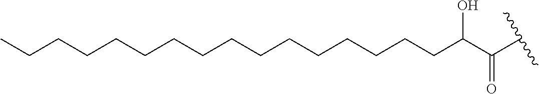 [SMILES: *C(=O)C(O)CCCCCCCCCCCCCCCC] |
| Lipid 19 | 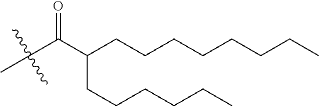 [SMILES: *C(=O)C(CCCCCC)CCCCCCCC] |
| Lipid 20 | 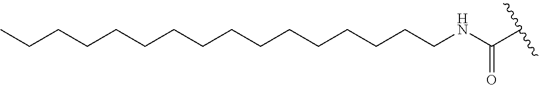 [SMILES: *C(=O)NCCCCCCCCCCCCCCCC] |
| Lipid 21 | 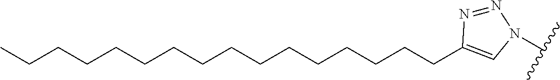 [SMILES: *n1cc(CCCCCCCCCCCCCCCC)nn1] |
| Lipid 22 | 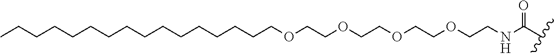 [SMILES: *C(=O)NCCOCCOCCOCCOCCCCCCCCCCCCCCCC] |
| Lipid 23 | 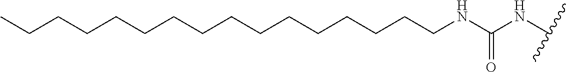 [SMILES: *NC(=O)NCCCCCCCCCCCCCCCC] |
| Lipid 24 | 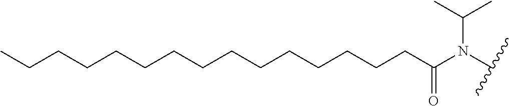 [SMILES: *N(C(=O)CCCCCCCCCCCCCCC)C(C)C] |

wherein [symbol] indicates a point of connection to L₁₃ or L₂₃.

67. The compound or pharmaceutically acceptable salt of any one of claims 52-59, wherein each of X and Y are independently selected from the group consisting of:

| Name | Structure |
|---|---|
| Lipid 1 | 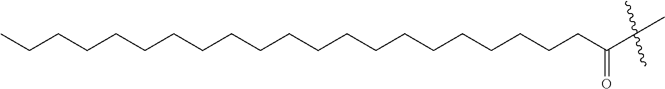 [SMILES: *C(=O)CCCCCCCCCCCCCCCCCCCCC] |
| Lipid 2 | 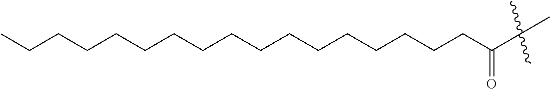 [SMILES: *C(=O)CCCCCCCCCCCCCCCCC] |
| Lipid 3 | 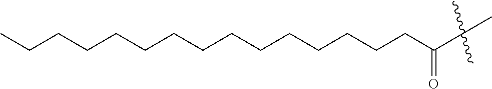 [SMILES: *C(=O)CCCCCCCCCCCCCCC] |
| Lipid 4 | 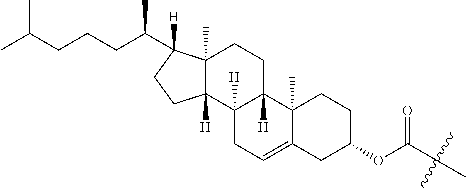 |
| Lipid 5 | 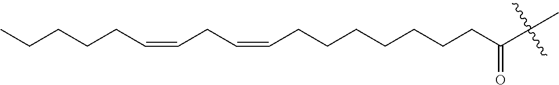 [SMILES: *C(=O)CCCCCCC/C=C\C/C=C\CCCCC] |
| Lipid 6 | 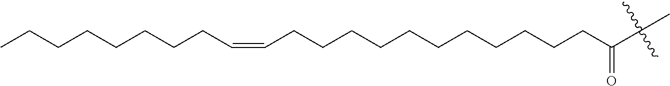 [SMILES: *C(=O)CCCCCCCCCCC/C=C\CCCCCCCC] |
| Lipid 7 | 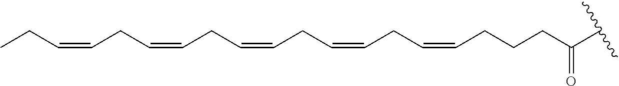 [SMILES: *C(=O)CCC/C=C\C/C=C\C/C=C\C/C=C\C/C=C\CC] |
| Lipid 8 | 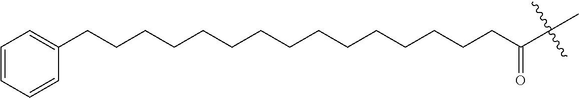 [SMILES: *C(=O)CCCCCCCCCCCCCCCc1ccccc1] |
| Lipid 9 | 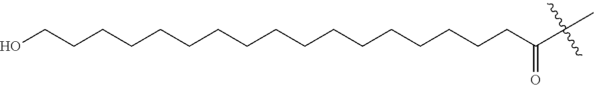 [SMILES: *C(=O)CCCCCCCCCCCCCCCCCO] |
| Lipid 10 | 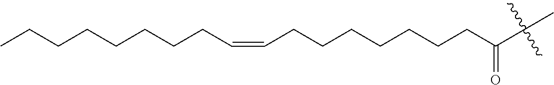 [SMILES: *C(=O)CCCCCCC/C=C\CCCCCCCC] |
| Lipid 11 | 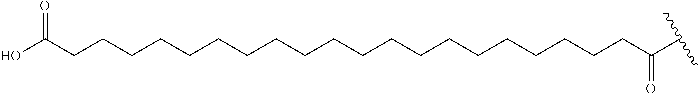 [SMILES: *C(=O)CCCCCCCCCCCCCCCCCCCCC(=O)O] |
| Lipid 12 | 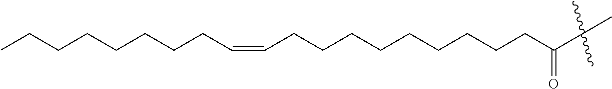 [SMILES: *C(=O)CCCCCCCCC/C=C\CCCCCCCC] |
| Lipid 14 | 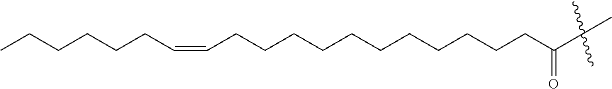 [SMILES: *C(=O)CCCCCCCCCCC/C=C\CCCCCC] |
| Lipid 15 | 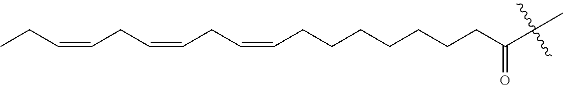 [SMILES: *C(=O)CCCCCCC/C=C\C/C=C\C/C=C\CC] |
| Lipid 16 | 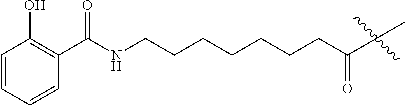 [SMILES: *C(=O)CCCCCCCNC(=O)c1ccccc1O] |
| Lipid 17 | 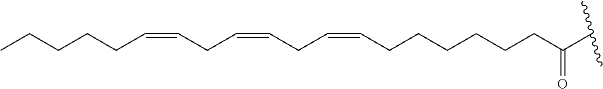 [SMILES: *C(=O)CCCCCC/C=C\C/C=C\C/C=C\CCCCC] |
| Lipid 18 | 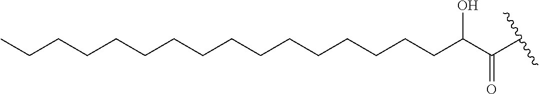 [SMILES: *C(=O)C(O)CCCCCCCCCCCCCCCC] |
| Lipid 19 | 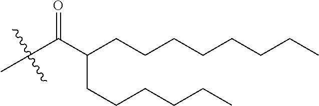 [SMILES: *C(=O)C(CCCCCC)CCCCCCCC] |
| Lipid 20 | 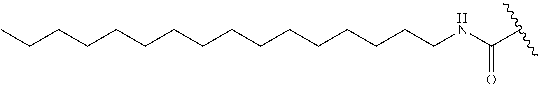 [SMILES: *C(=O)NCCCCCCCCCCCCCCCC] |
| Lipid 21 | 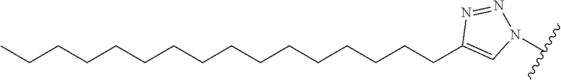 [SMILES: *n1cc(CCCCCCCCCCCCCCCC)nn1] |
| Lipid 22 | 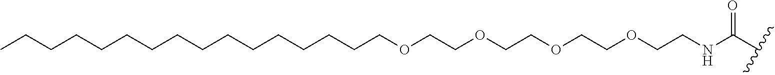 [SMILES: *C(=O)NCCOCCOCCOCCOCCCCCCCCCCCCCCCC] |
| Lipid 23 | 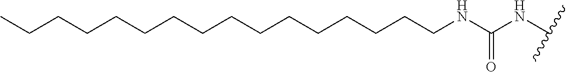 [SMILES: *NC(=O)NCCCCCCCCCCCCCCCC] |
| Lipid 24 | 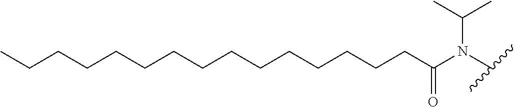 [SMILES: *N(C(=O)CCCCCCCCCCCCCCC)C(C)C] |

wherein [symbol] indicates a point of connection to L₁₃ or L₂₃.

68. The compound or pharmaceutically acceptable salt of claim 67, wherein each of X and Y are independently selected from the group consisting of:

| Name | Structure |
|---|---|
| Lipid 3 | 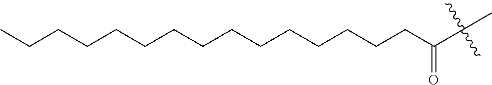 [SMILES: *C(=O)CCCCCCCCCCCCCCC] |
| Lipid 19 | 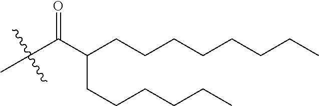 [SMILES: *C(=O)C(CCCCCC)CCCCCCCC] |

wherein [symbol] indicates a point of connection to L₁₃ or L₂₃.

69. The compound or pharmaceutically acceptable salt ofany one of claims 52-68, wherein L_{A3} is selected from the group consisting of:

| Name | Structure |
|---|---|
| Tether 1-3 | 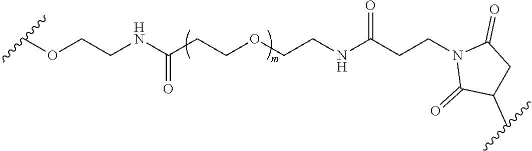 |
| Tether 2-3 | 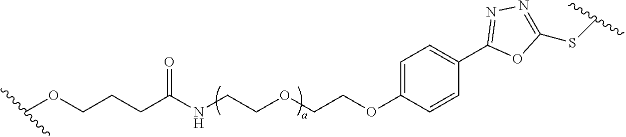 |
| Tether 3-3 | 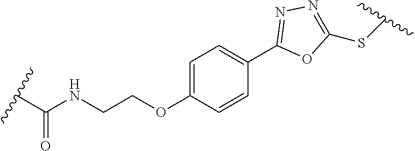 [SMILES: *Sc1nnc(-c2ccc(OCCNC(*)=O)cc2)o1] |
| Tether 4-3 | 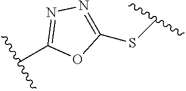 [SMILES: *Sc1nnc(*)o1] |
| Tether 5-3 | 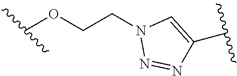 [SMILES: *OCCn1cc(*)nn1] |

wherein each of m and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and
each [symbol] indicates a point of connection to R_{Z} or the phenyl ring of Formula (III).

70. The compound or pharmaceutically acceptable salt of claim 69, wherein
m is 1, 2, 3, 4, or 5; and
a is 2, 3, 4, or 5.

71. The compound or pharmaceutically acceptable salt of any one of claims 52-70, wherein each of R¹ and R² is independently hydrogen or C₁₋₃ alkyl.

72. The compound or pharmaceutically acceptable salt of any one of claims 52-71, wherein each of R¹ and R² is hydrogen.

73. The compound or pharmaceutically acceptable salt of claim 52, wherein the compound of Formula (III) is selected from the group consisting of: 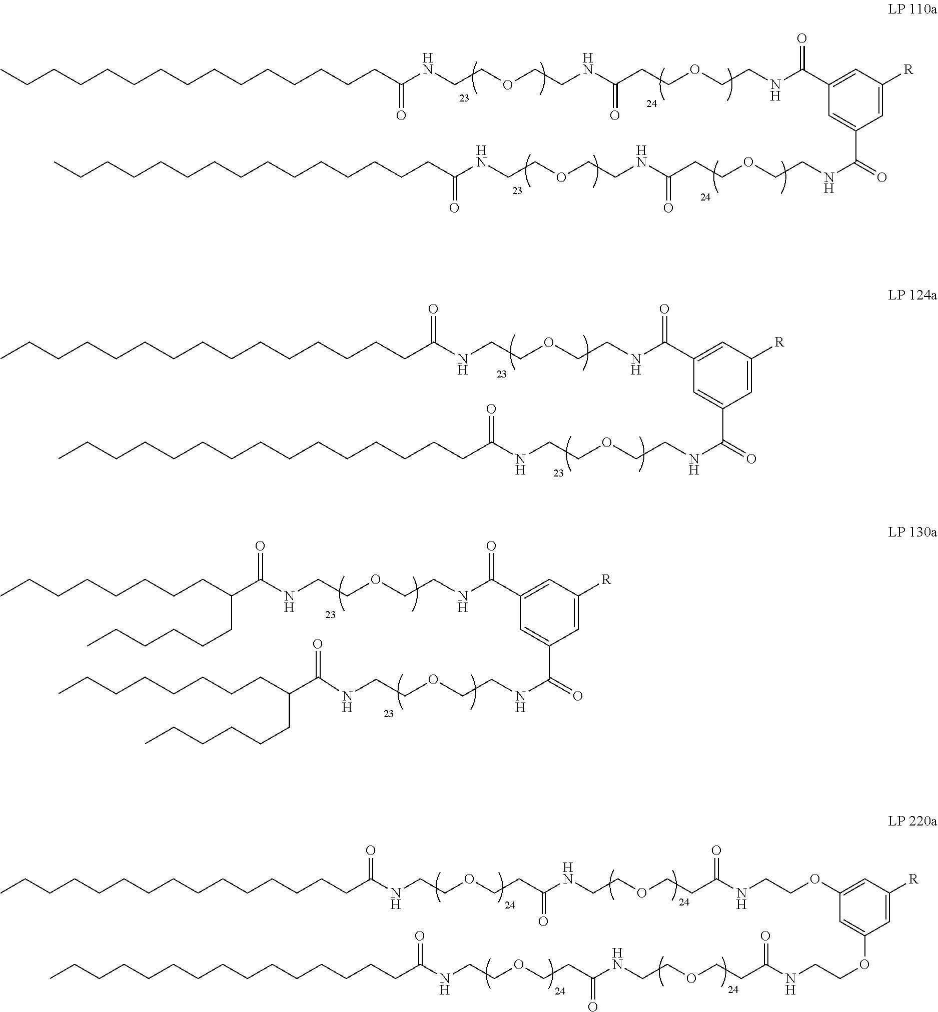
or a pharmaceutically acceptable salt of any of these compounds.

74. The compound or pharmaceutically acceptable salt of claim 52, wherein the compound of formula (III) is selected from the group consisting of: 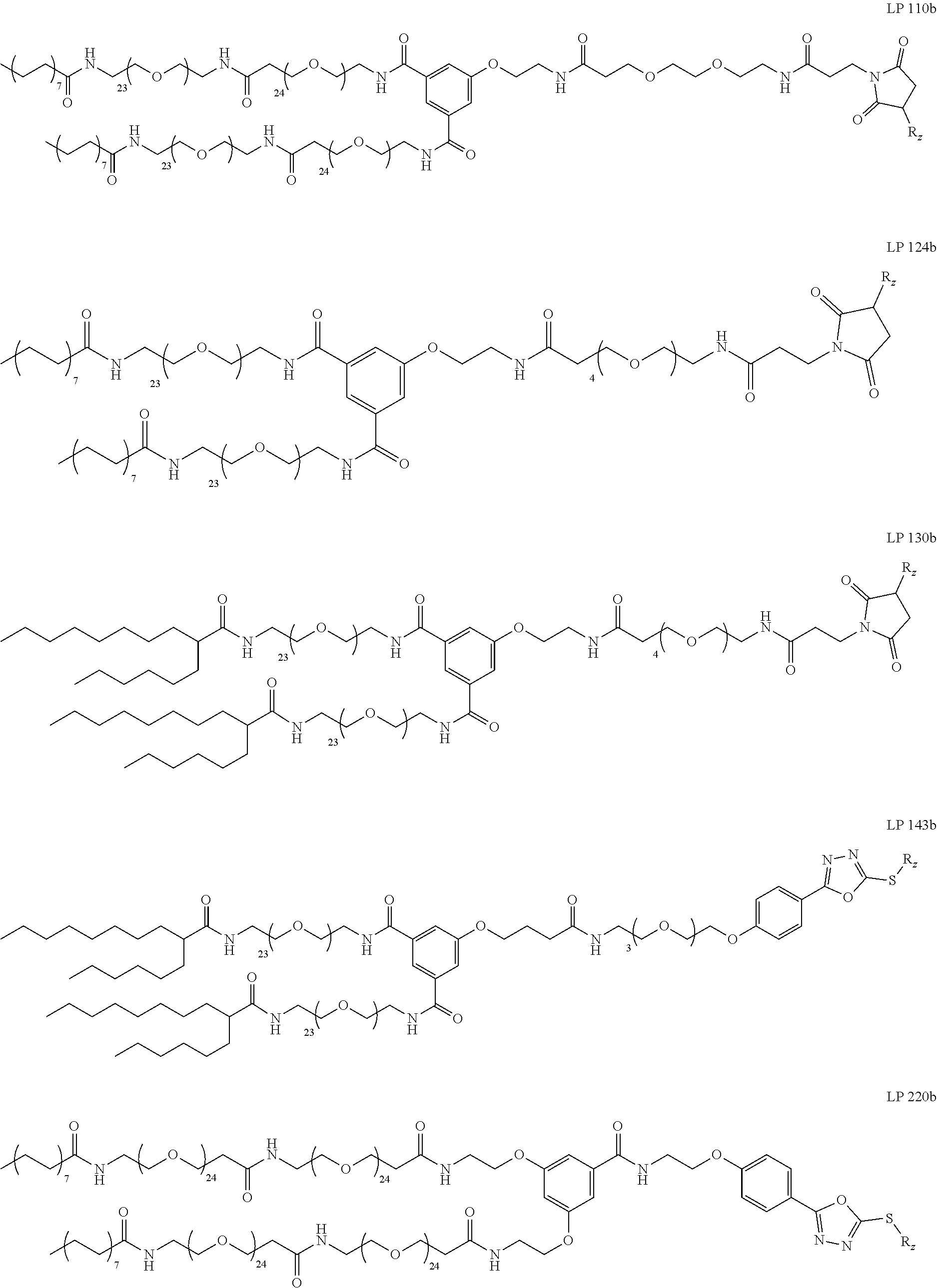 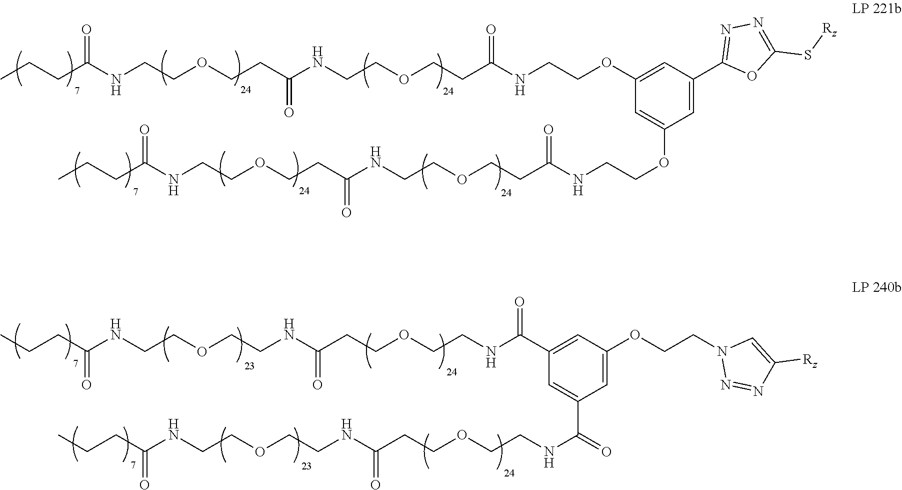
or a pharmaceutically acceptable salt of any of these compounds.

75. The compound or pharmaceutically acceptable salt of claim 52, wherein the compound of Formula (III) is a compound of Formula (IIIa): 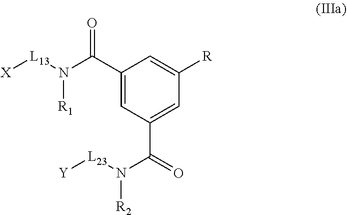
or a pharmaceutically acceptable salt thereof, wherein
R is L_{A3}-R_{Z};
L_{A3} is a bond or a bivalent moiety connecting R_{Z} to the phenyl ring;
R_{Z} comprises an oligonucleotide-based agent;
R₁ and R₂ are each independently hydrogen or C₁₋₆ alkyl;
L₁₃ and L₂₃ are each independently linkers comprising at least about 5 PEG units; and
X and Y are each independently lipids comprising from about 10 to about 50 carbon atoms.

76. The compound or pharmaceutically acceptable salt of claim 75, wherein each of L₁₃ and L₂₃ is independently selected from the group consisting of:

| Name | Structure |
|---|---|
| Linker 1-3 | 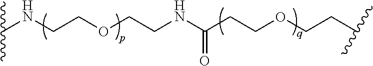 |
| Linker 2-3 | 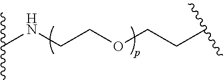 |

wherein
p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
each [symbol] indicates a point of connection to X, Y, —NR¹—, or —NR²—, provided that
(i) in Linker 1-3, p+q≥5, and
(ii) in Linker 2-3, p≥5.

77. The compound or pharmaceutically acceptable salt of claim 75 or claim 76, wherein each of X and Y are independently selected from the group consisting of:

| Name | Structure |
|---|---|
| Lipid 3 | 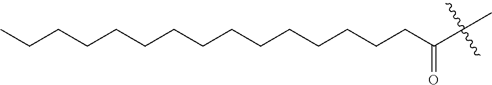 [SMILES: *C(=O)CCCCCCCCCCCCCCC] |
| Lipid 19 | 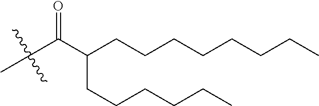 [SMILES: *C(=O)C(CCCCCC)CCCCCCCC] |

wherein [symbol] indicates a point of connection to L₁₃ or L₂₃.

78. The compound or pharmaceutically acceptable salt of any one of claims 75-77, wherein L_{A3} is selected from the group consisting of:

| Name | Structure |
|---|---|
| Tether 1-3 | 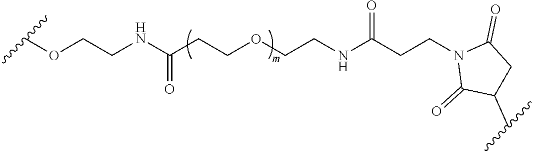 |
| Tether 2-3 | 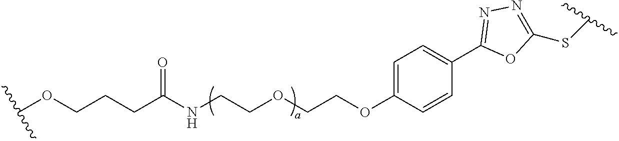 |
| Tether 5-3 | 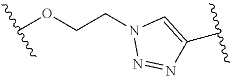 [SMILES: *OCCn1cc(*)nn1] |

wherein each of m and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and
each [symbol] indicates a point of connection to R_{Z} or the phenyl ring of Formula (IIIa).

79. The compound or pharmaceutically acceptable salt of any one of claims 75-78, wherein each of R¹ and R² is independently hydrogen or C₁₋₃ alkyl.

80. The compound or pharmaceutically acceptable salt of any one of claims 75-79, wherein each of R¹ and R₂ is hydrogen.

81. The compound or pharmaceutically acceptable salt of claim 75, wherein the compound of Formula (IIIa) selected from the group consisting of: 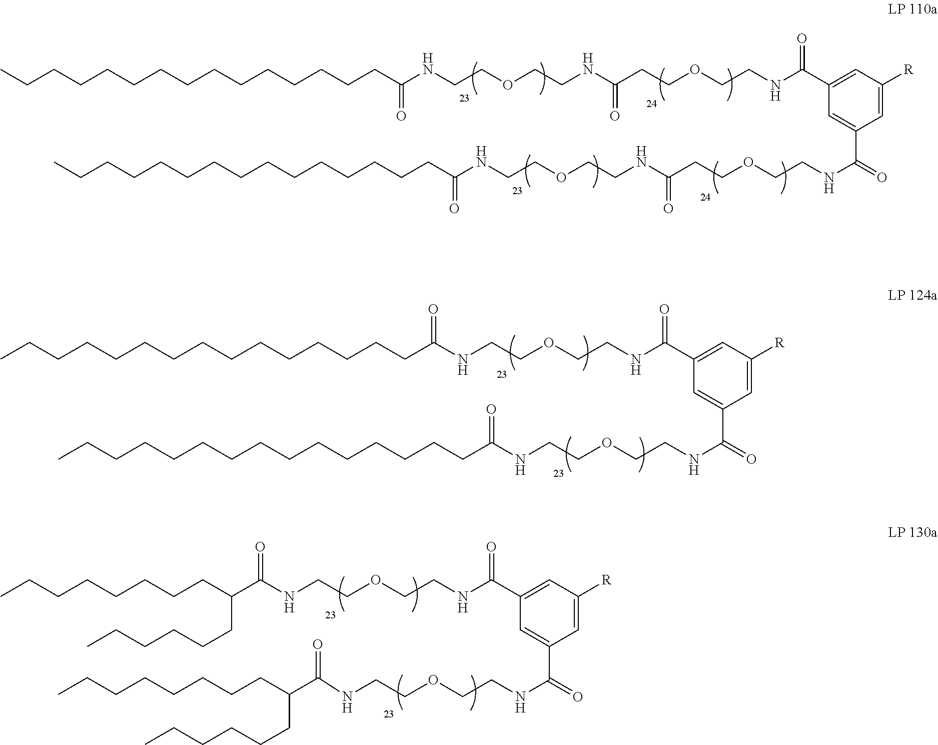
or a pharmaceutically acceptable salt of any of these compounds.

82. The compound or pharmaceutically acceptable salt of claim 75, or a pharmaceutically acceptable salt thereof consisting of: 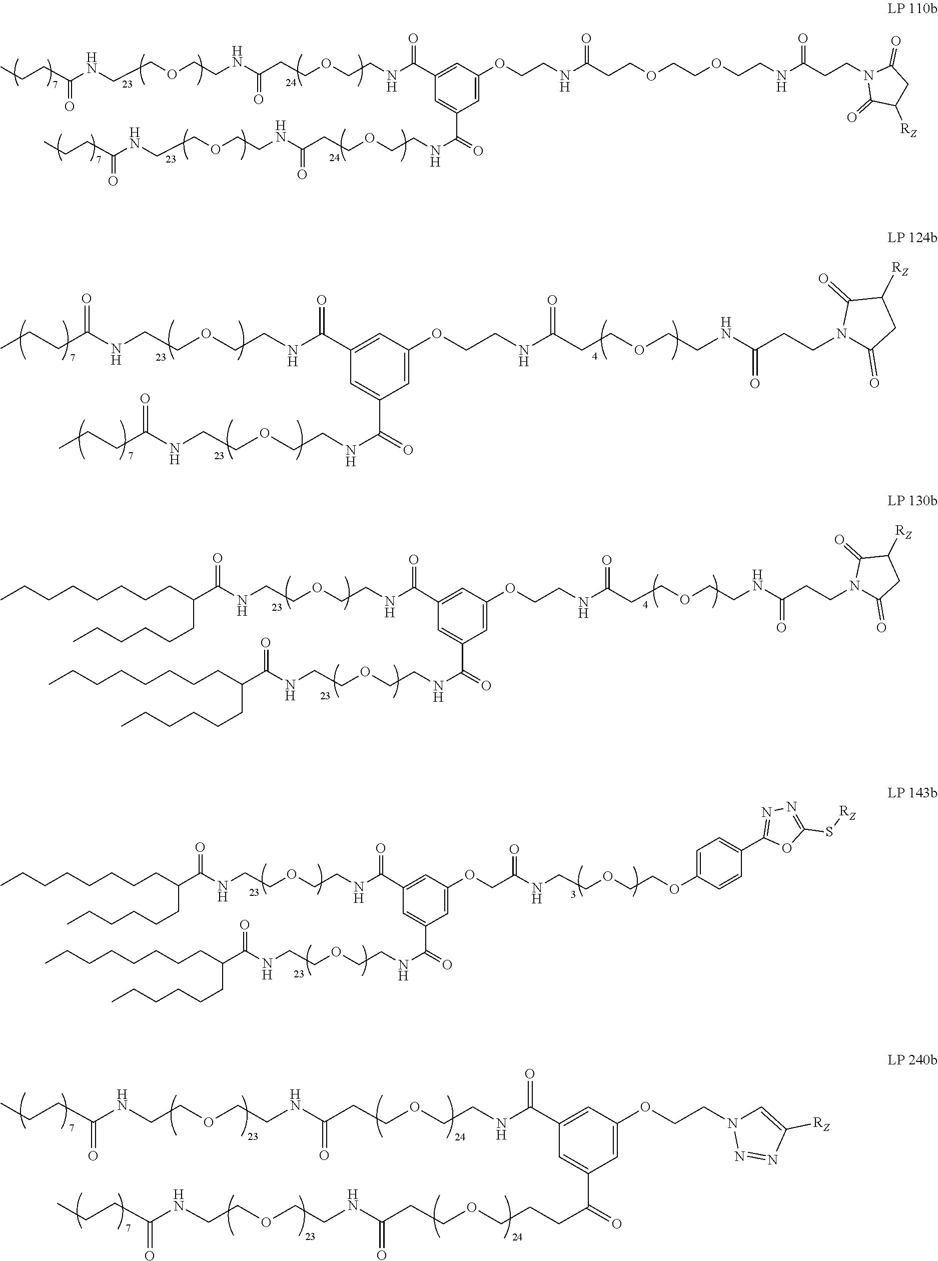
or a pharmaceutically acceptable salt of any of these compounds.

83. The compound or pharmaceutically acceptable salt of claim 52, wherein the compound of Formula (III) is a compound of Formula (IIIb): 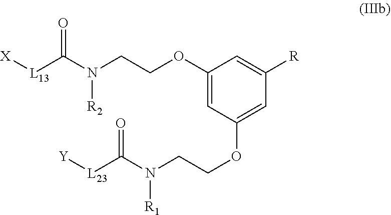
or a pharmaceutically acceptable salt thereof, wherein
R is L_{A3}-R_{Z};
L_{A3} is a bond or a bivalent moiety connecting R_{Z} to the phenyl of Formula (IIIb);
R_{Z} comprises an oligonucleotide-based agent;
R₁ and R₂ are each independently selected from hydrogen or C₁₋₆ alkyl;
L₁₃ and L₂₃ are each independently linkers comprising at least about 5 PEG units; and
X and Y are each independently lipids comprising from about 10 to about 50 carbon atoms.

84. The compound or pharmaceutically acceptable salt of claim 83, wherein each of L₁₃ and L₂₃ is

| Name | Structure |
|---|---|
| Linker 3-3 | 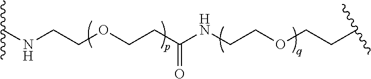 |

wherein
p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
each [symbol] indicates a point of connection to X, Y, or —C(O)—, provided that in Linker 3-3, p+q≥5.

85. The compound of claim 83 or 84, wherein each of X and Y are independently:

| Name | Structure |
|---|---|
| Lipid 3 | 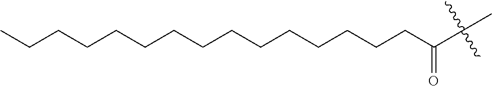 [SMILES: *C(=O)CCCCCCCCCCCCCCC] |

wherein [symbol] indicates a point of connection to L₁₃ or L₂₃.

86. The compound or pharmaceutically acceptable salt of any one of claims 83-85, wherein L_{A3} is selected from the group consisting of:

| Name | Structure |
|---|---|
| Tether 3-3 | 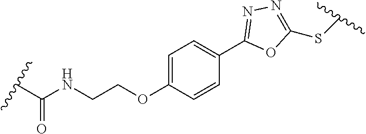 [SMILES: *Sc1nnc(-c2ccc(OCCNC(*)=O)cc2)o1] |
| Tether 4-3 | 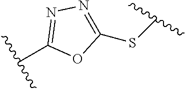 [SMILES: *Sc1nnc(*)o1] |

wherein each [symbol] indicates a point of connection to R_{Z} or the phenyl ring of Formula (IIIb).

87. The compound or pharmaceutically acceptable salt of any one of claims 83-86, wherein each of R¹ and R² is independently hydrogen or C₁₋₃ alkyl.

88. The compound or pharmaceutically acceptable salt of any one of claims 83-87, wherein each of R¹ and R² is hydrogen.

89. The compound or pharmaceutically acceptable salt of claim 83, wherein the compound of Formula (IIIb) is selected from the group consisting of: 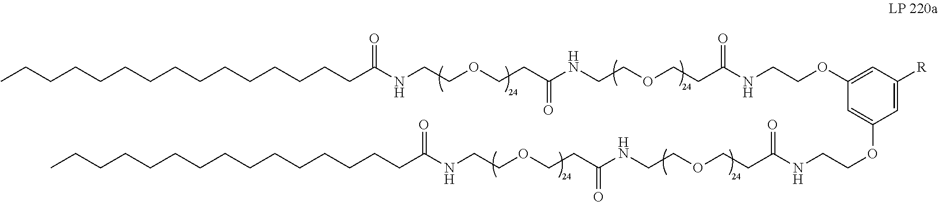
or a pharmaceutically acceptable salt of any of these compounds.

90. The compound or pharmaceutically acceptable salt of claim 83, wherein the compound of Formula (IIIb) is selected from the group consisting of: 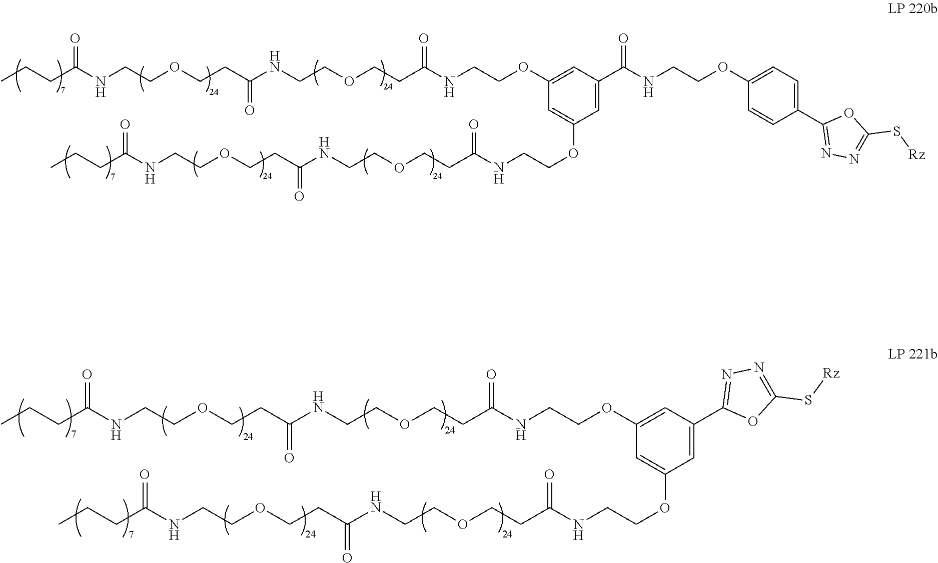
or a pharmaceutically acceptable salt of any of these compounds.

91. The compound or pharmaceutically acceptable salt of any one of claims 52-90, wherein the oligonucleotide-based agent is an RNAi agent.

92. A compound of Formula (IV): 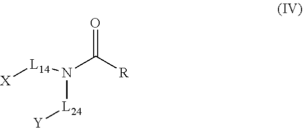
or a pharmaceutically acceptable salt thereof, wherein
R is L_{A4}-R_{Z};
L_{A4} is a bond or a bivalent moiety connecting R_{Z} to —C(O)—;
R_{Z} comprises an oligonucleotide-based agent;
L₁₄ and L₂₄ are each independently linkers comprising at least about 5 PEG units; and
X and Y are each independently lipids comprising from about 10 to about 50 carbon atoms.

93. The compound or pharmaceutically acceptable salt of claim 92, wherein L₁₄ and L₂₄ each comprise about 15 to about 100 PEG units.

94. The compound or pharmaceutically acceptable salt of claim 92 or claim 93, wherein L₁₄ and L₂₄ each independently comprise about 20 to about 60 PEG units.

95. The compound or pharmaceutically acceptable salt of any one of claims 92-94, wherein L₁₄ and L₂₄ each independently comprise about 20 to about 30 PEG units.

96. The compound or pharmaceutically acceptable salt of any one of claims 92-94, wherein L₁₄ and L₂₄ each independently comprise about 40 to about 60 PEG units.

97. The compound or pharmaceutically acceptable salt of claim 92, wherein one of L₁₄ and L₂₄ comprises about 20 to about 30 PEG units and the other comprises about 40 to about 60 PEG units.

98. The compound or pharmaceutically acceptable salt of claim 92, wherein each of L₁₄ and L₂₄ is independently selected from the group consisting of:

| Name | Structure |
|---|---|
| Linker 1-4 | 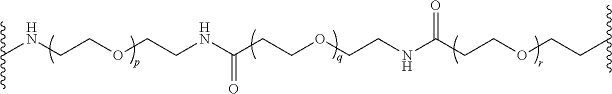 |
| Linker 2-4 | 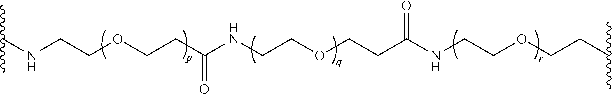 |
| Linker 3-4 | 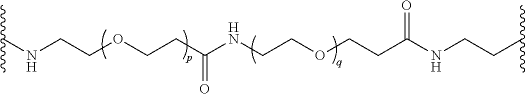 |
| Linker 4-4 | 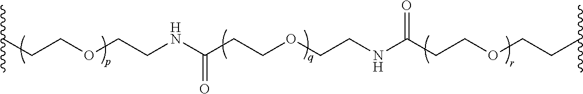 |
| Linker 5-4 | 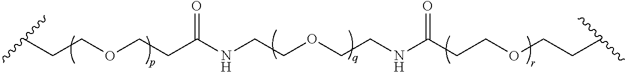 |

wherein
each p is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30;
each q is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30;
each r is independently 1, 2, 3, 4, 5, 6, 7, 8, 9, or 10; and
each [symbol] indicates a point of connection to X, Y, or —N—C(O)— of Formula (IV); provided that
(i) in Linker 1-4, Linker 2-4, and Linker 4-4, p+q+r≥5; and
(ii) in Linker 3-4, p+q≥5.

99. The compound or pharmaceutically acceptable salt of claim 98, wherein
each p is independently 20, 21, 22, 23, 24, or 25;
each q is independently 20, 21, 22, 23, 24, or 25; and
each r is independently 2, 3, 4, 5, or 6.

100. The compound or pharmaceutically acceptable salt of any one of claims 92-99, wherein at least one of X and Y is an unsaturated lipid.

101. The compound or pharmaceutically acceptable salt of any one of claims 92-100, wherein at least one of X and Y is a saturated lipid.

102. The compound or pharmaceutically acceptable salt of any one of claims 92-101, wherein at least one of X and Y is a branched lipid.

103. The compound or pharmaceutically acceptable salt of any one of claims 92-102, wherein at least one of X and Y is a straight chain lipid.

104. The compound or pharmaceutically acceptable salt of any one of claims 92-103, wherein at least one of X and Y is a lipid comprising from about 10 to about 25 carbon atoms.

105. The compound or pharmaceutically acceptable salt of any one of claims 92-104, wherein at least one of X and Y is cholesteryl.

106. The compound or pharmaceutically acceptable salt of any one of claims 92-99, wherein at least one of X and Y is selected from the group consisting of:

| Name | Structure |
|---|---|
| Lipid 1 | 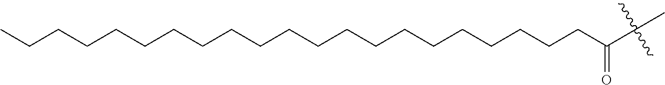 [SMILES: *C(=O)CCCCCCCCCCCCCCCCCCCCC] |
| Lipid 2 | 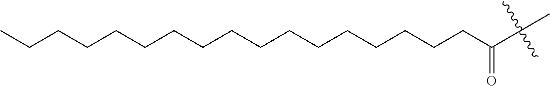 [SMILES: *C(=O)CCCCCCCCCCCCCCCCC] |
| Lipid 3 | 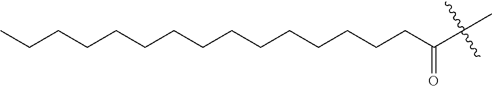 [SMILES: *C(=O)CCCCCCCCCCCCCCC] |
| Lipid 4 | 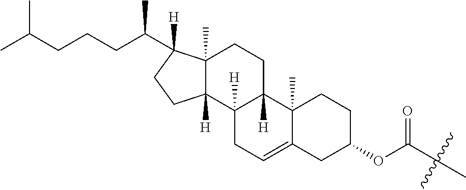 |
| Lipid 5 | 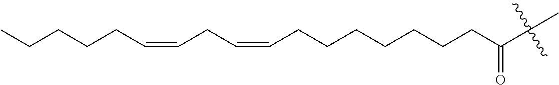 [SMILES: *C(=O)CCCCCCC/C=C\C/C=C\CCCCC] |
| Lipid 6 | 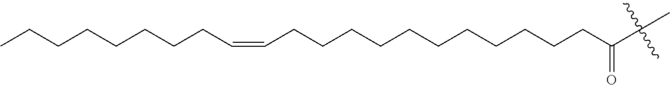 [SMILES: *C(=O)CCCCCCCCCCC/C=C\CCCCCCCC] |
| Lipid 7 | 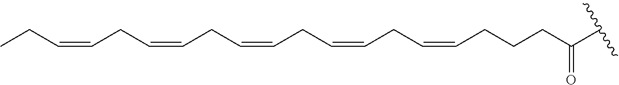 [SMILES: *C(=O)CCC/C=C\C/C=C\C/C=C\C/C=C\C/C=C\CC] |
| Lipid 8 | 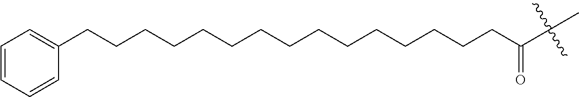 [SMILES: *C(=O)CCCCCCCCCCCCCCCc1ccccc1] |
| Lipid 9 | 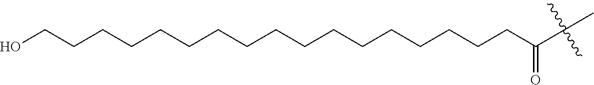 [SMILES: *C(=O)CCCCCCCCCCCCCCCCCO] |
| Lipid 10 | 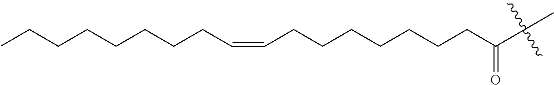 [SMILES: *C(=O)CCCCCCC/C=C\CCCCCCCC] |
| Lipid 11 | 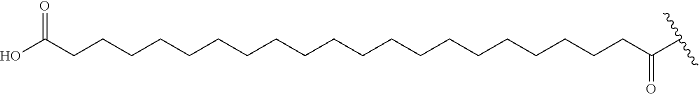 [SMILES: *C(=O)CCCCCCCCCCCCCCCCCCCCC(=O)O] |
| Lipid 12 | 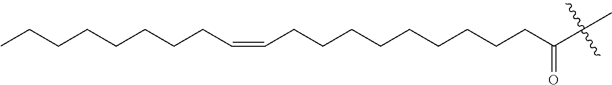 [SMILES: *C(=O)CCCCCCCCC/C=C\CCCCCCCC] |
| Lipid 14 | 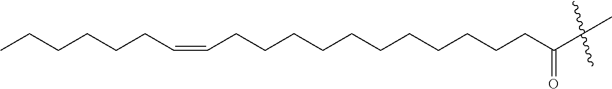 [SMILES: *C(=O)CCCCCCCCCCC/C=C\CCCCCC] |
| Lipid 15 | 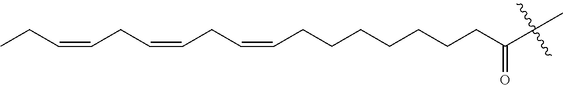 [SMILES: *C(=O)CCCCCCC/C=C\C/C=C\C/C=C\CC] |
| Lipid 16 | 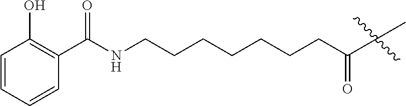 [SMILES: *C(=O)CCCCCCCNC(=O)c1ccccc1O] |
| Lipid 17 | 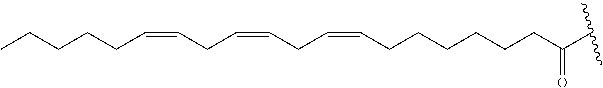 [SMILES: *C(=O)CCCCCC/C=C\C/C=C\C/C=C\CCCCC] |
| Lipid 18 | 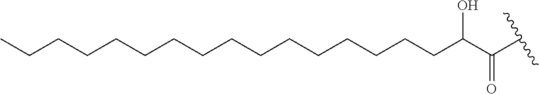 [SMILES: *C(=O)C(O)CCCCCCCCCCCCCCCC] |
| Lipid 19 | 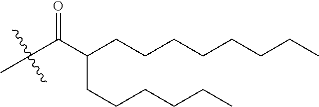 [SMILES: *C(=O)C(CCCCCC)CCCCCCCC] |
| Lipid 20 | 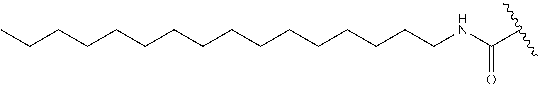 [SMILES: *C(=O)NCCCCCCCCCCCCCCCC] |
| Lipid 21 | 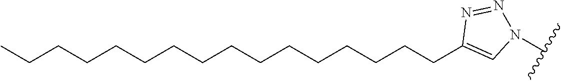 [SMILES: *n1cc(CCCCCCCCCCCCCCCC)nn1] |
| Lipid 22 | 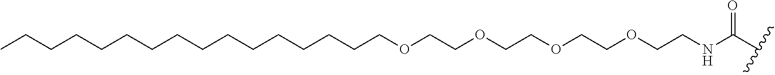 [SMILES: *C(=O)NCCOCCOCCOCCOCCCCCCCCCCCCCCCC] |
| Lipid 23 | 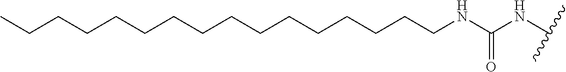 [SMILES: *NC(=O)NCCCCCCCCCCCCCCCC] |
| Lipid 24 | 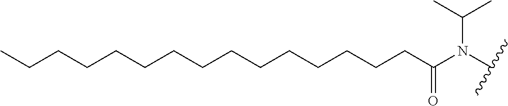 [SMILES: *N(C(=O)CCCCCCCCCCCCCCC)C(C)C] |

wherein [symbol] indicates a point of connection to L₁₄ or L₂₄.

107. The compound or pharmaceutically acceptable salt of any one of claims 92-99, wherein each of X and Y are independently selected from the group consisting of:

| Name | Structure |
|---|---|
| Lipid 1 | 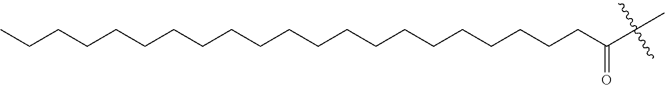 [SMILES: *C(=O)CCCCCCCCCCCCCCCCCCCCC] |
| Lipid 2 | 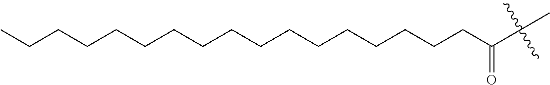 [SMILES: *C(=O)CCCCCCCCCCCCCCCCC] |
| Lipid 3 | 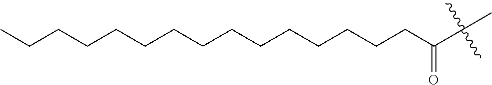 [SMILES: *C(=O)CCCCCCCCCCCCCCC] |
| Lipid 4 | 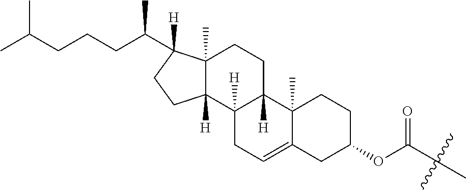 |
| Lipid 5 | 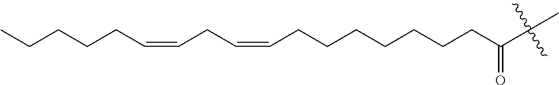 [SMILES: *C(=O)CCCCCCC/C=C\C/C=C\CCCCC] |
| Lipid 6 | 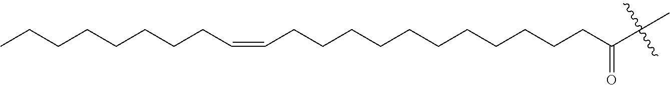 [SMILES: *C(=O)CCCCCCCCCCC/C=C\CCCCCCCC] |
| Lipid 7 | 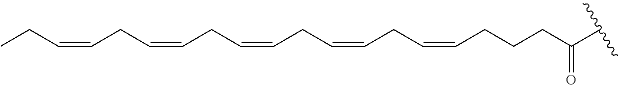 [SMILES: *C(=O)CCC/C=C\C/C=C\C/C=C\C/C=C\C/C=C\CC] |
| Lipid 8 | 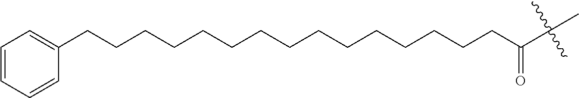 [SMILES: *C(=O)CCCCCCCCCCCCCCCc1ccccc1] |
| Lipid 9 | 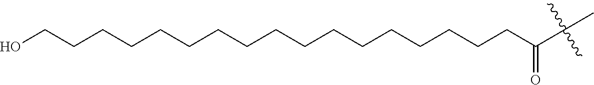 [SMILES: *C(=O)CCCCCCCCCCCCCCCCCO] |
| Lipid 10 | 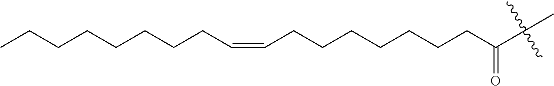 [SMILES: *C(=O)CCCCCCC/C=C\CCCCCCCC] |
| Lipid 11 | 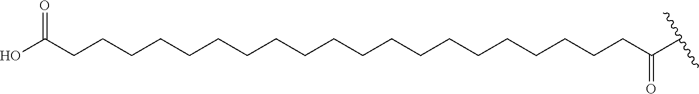 [SMILES: *C(=O)CCCCCCCCCCCCCCCCCCCCC(=O)O] |
| Lipid 12 | 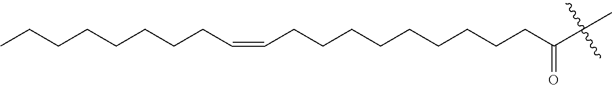 [SMILES: *C(=O)CCCCCCCCC/C=C\CCCCCCCC] |
| Lipid 14 | 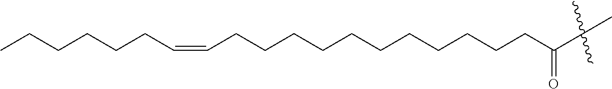 [SMILES: *C(=O)CCCCCCCCCCC/C=C\CCCCCC] |
| Lipid 15 | 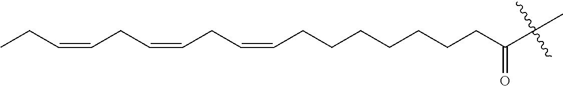 [SMILES: *C(=O)CCCCCCC/C=C\C/C=C\C/C=C\CC] |
| Lipid 16 | 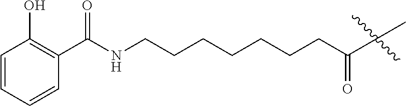 [SMILES: *C(=O)CCCCCCCNC(=O)c1ccccc1O] |
| Lipid 17 | 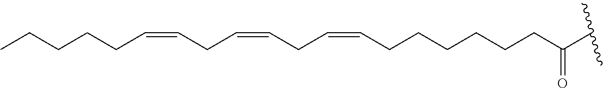 [SMILES: *C(=O)CCCCCC/C=C\C/C=C\C/C=C\CCCCC] |
| Lipid 18 | 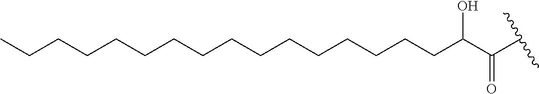 [SMILES: *C(=O)C(O)CCCCCCCCCCCCCCCC] |
| Lipid 19 | 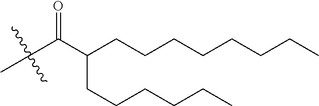 [SMILES: *C(=O)C(CCCCCC)CCCCCCCC] |
| Lipid 20 | 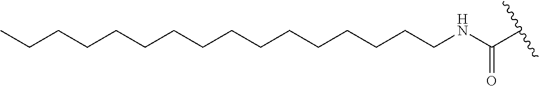 [SMILES: *C(=O)NCCCCCCCCCCCCCCCC] |
| Lipid 21 | 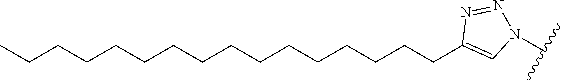 [SMILES: *n1cc(CCCCCCCCCCCCCCCC)nn1] |
| Lipid 22 | 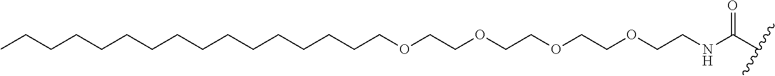 [SMILES: *C(=O)NCCOCCOCCOCCOCCCCCCCCCCCCCCCC] |
| Lipid 23 | 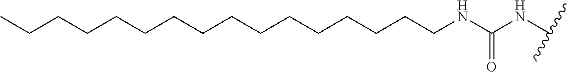 [SMILES: *NC(=O)NCCCCCCCCCCCCCCCC] |
| Lipid 24 | 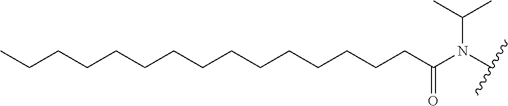 [SMILES: *N(C(=O)CCCCCCCCCCCCCCC)C(C)C] |

wherein [symbol] indicates a point of connection to L₁₄ or L₂₄.

108. The compound or pharmaceutically acceptable salt of claim 107, wherein each of X and Y are independently selected from the group consisting of:

| Name | Structure |
|---|---|
| Lipid 1 | 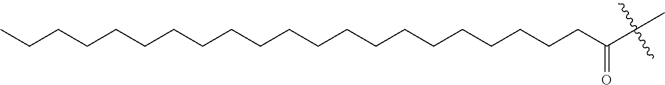 [SMILES: *C(=O)CCCCCCCCCCCCCCCCCCCCC] |
| Lipid 2 | 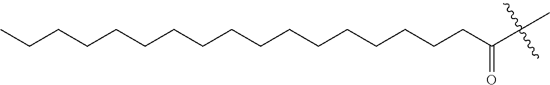 [SMILES: *C(=O)CCCCCCCCCCCCCCCCC] |
| Lipid 3 | 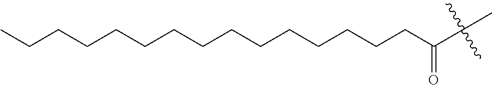 [SMILES: *C(=O)CCCCCCCCCCCCCCC] |
| Lipid 5 | 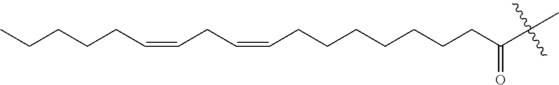 [SMILES: *C(=O)CCCCCCC/C=C\C/C=C\CCCCC] |
| Lipid 8 | 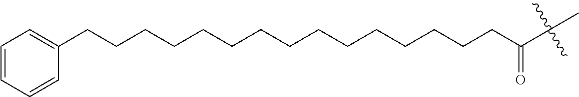 [SMILES: *C(=O)CCCCCCCCCCCCCCCc1ccccc1] |
| Lipid 9 | 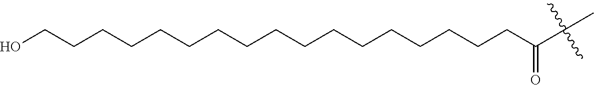 [SMILES: *C(=O)CCCCCCCCCCCCCCCCCO] |
| Lipid 10 | 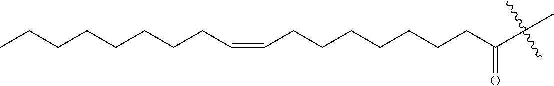 [SMILES: *C(=O)CCCCCCC/C=C\CCCCCCCC] |
| Lipid 11 | 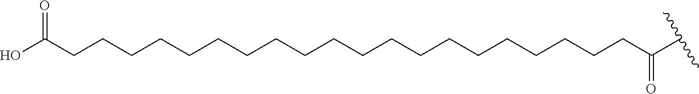 [SMILES: *C(=O)CCCCCCCCCCCCCCCCCCCCC(=O)O] |
| Lipid 12 | 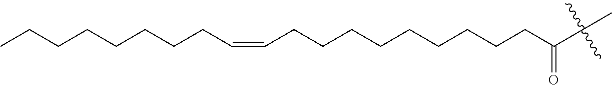 [SMILES: *C(=O)CCCCCCCCC/C=C\CCCCCCCC] |
| Lipid 15 | 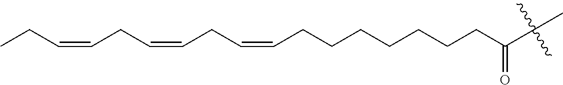 [SMILES: *C(=O)CCCCCCC/C=C\C/C=C\C/C=C\CC] |
| Lipid 16 | 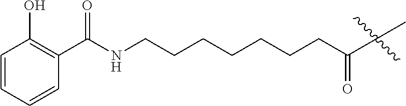 [SMILES: *C(=O)CCCCCCCNC(=O)c1ccccc1O] |
| Lipid 17 | 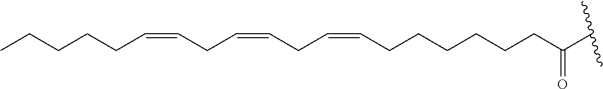 [SMILES: *C(=O)CCCCCC/C=C\C/C=C\C/C=C\CCCCC] |
| Lipid 18 | 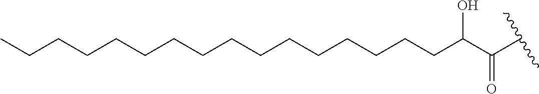 [SMILES: *C(=O)C(O)CCCCCCCCCCCCCCCC] |
| Lipid 19 | 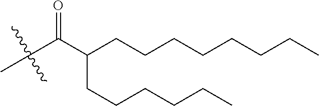 [SMILES: *C(=O)C(CCCCCC)CCCCCCCC] |
| Lipid 20 | 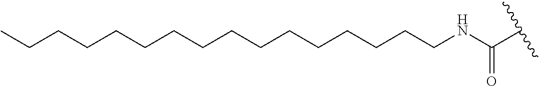 [SMILES: *C(=O)NCCCCCCCCCCCCCCCC] |
| Lipid 21 | 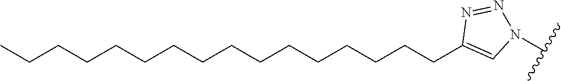 [SMILES: *n1cc(CCCCCCCCCCCCCCCC)nn1] |
| Lipid 22 | 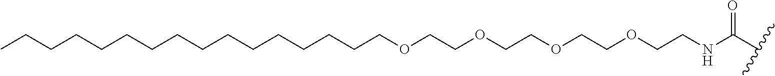 [SMILES: *C(=O)NCCOCCOCCOCCOCCCCCCCCCCCCCCCC] |
| Lipid 23 | 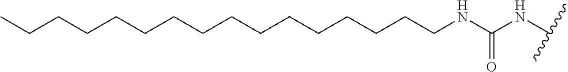 [SMILES: *NC(=O)NCCCCCCCCCCCCCCCC] |
| Lipid 24 | 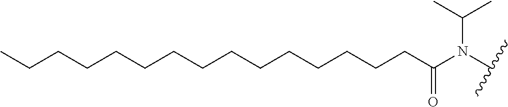 [SMILES: *N(C(=O)CCCCCCCCCCCCCCC)C(C)C] |

wherein [symbol] indicates a point of connection to L₁₄ or L₂₄.

109. The compound or pharmaceutically acceptable salt of any one of claims 92-109, wherein L_{A4} is selected from the group consisting of:

| Name | Structure |
|---|---|
| Tether 1-4 | 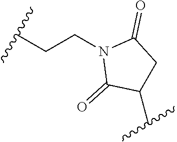 [SMILES: *CCN1C(=O)CC(*)C1=O] |
| Tether 2-4 | 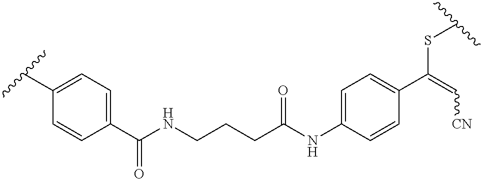 [SMILES: *SC(=CC#N)c1ccc(NC(=O)CCCNC(=O)c2ccc(*)cc2)cc1] |
| Tether 3-4 | 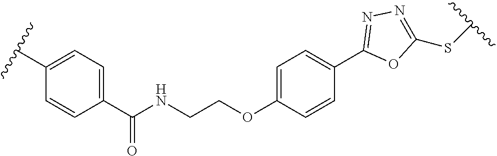 [SMILES: *Sc1nnc(-c2ccc(OCCNC(=O)c3ccc(*)cc3)cc2)o1] |
| Tether 4-4 | 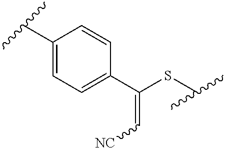 [SMILES: *SC(=CC#N)c1ccc(*)cc1] |
| Tether 5-4 | 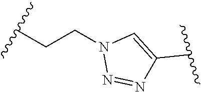 [SMILES: *CCn1cc(*)nn1] |
| Tether 6-4 | 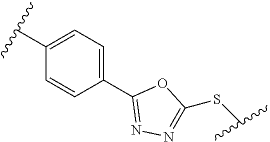 [SMILES: *Sc1nnc(-c2ccc(*)cc2)o1] |

wherein each [symbol] indicates a point of connection to R_{Z} or the —C(O)— of Formula (IV).

110. The compound or pharmaceutically acceptable salt of claim 92, wherein the compound of Formula (IV) is selected from the group consisting of: 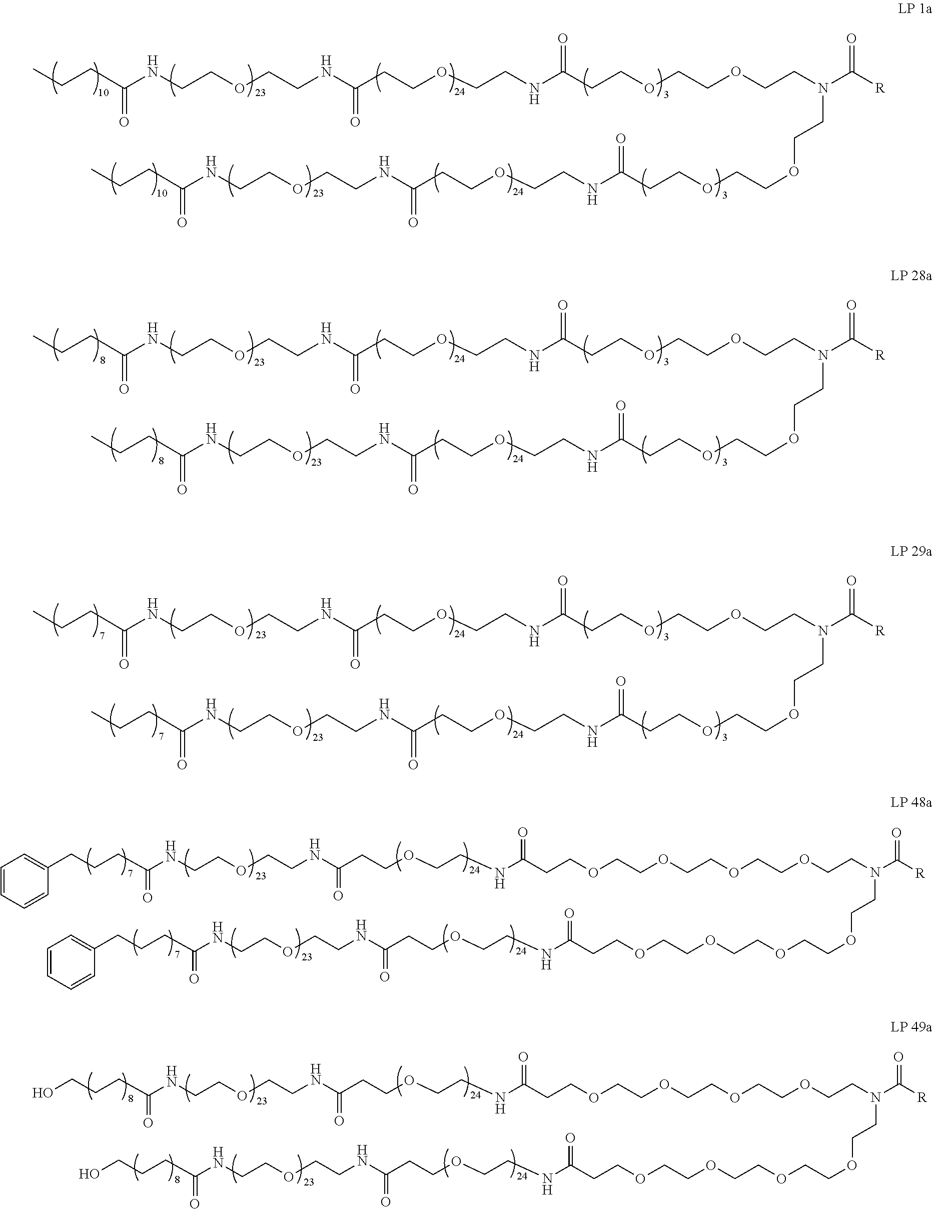 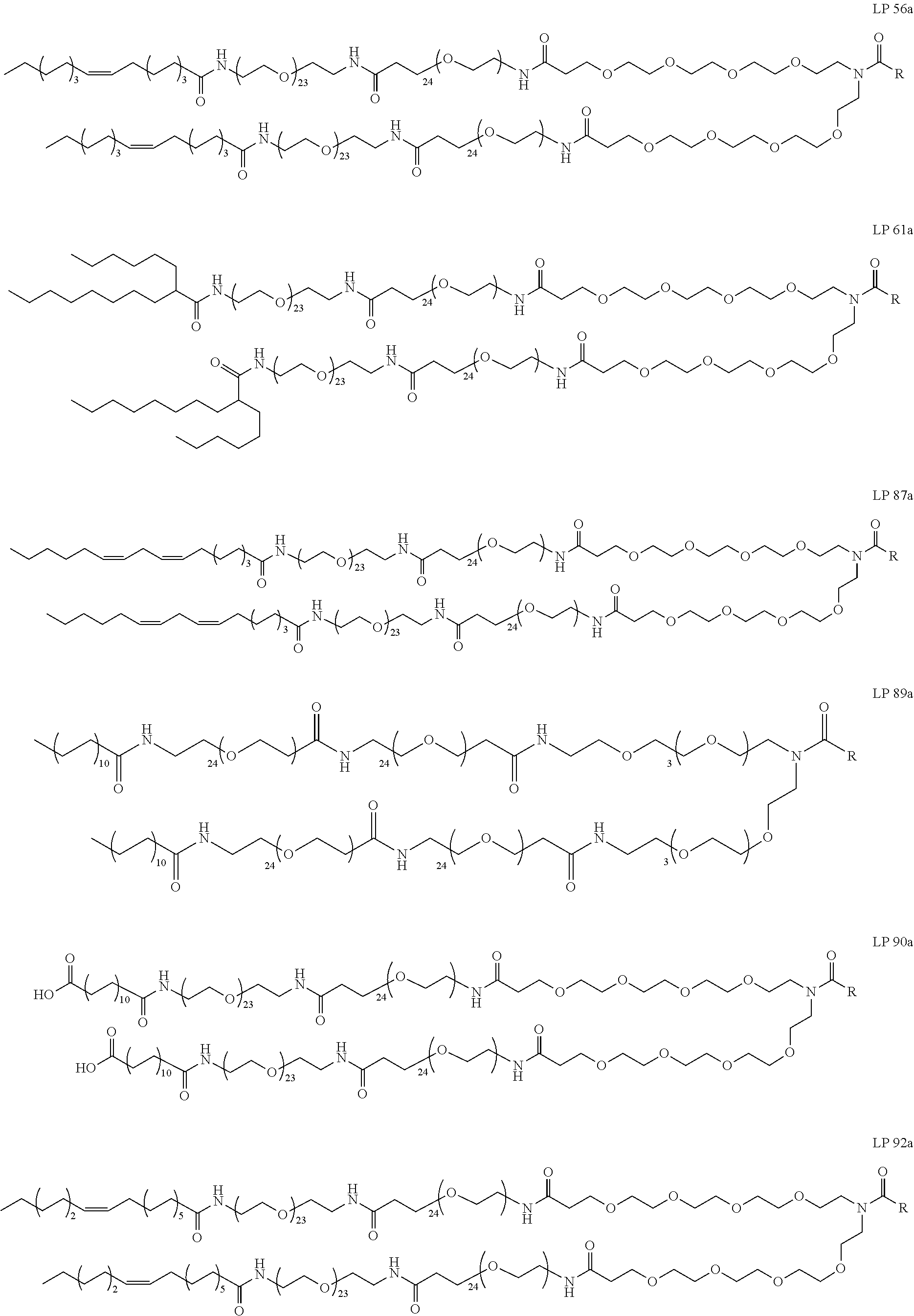 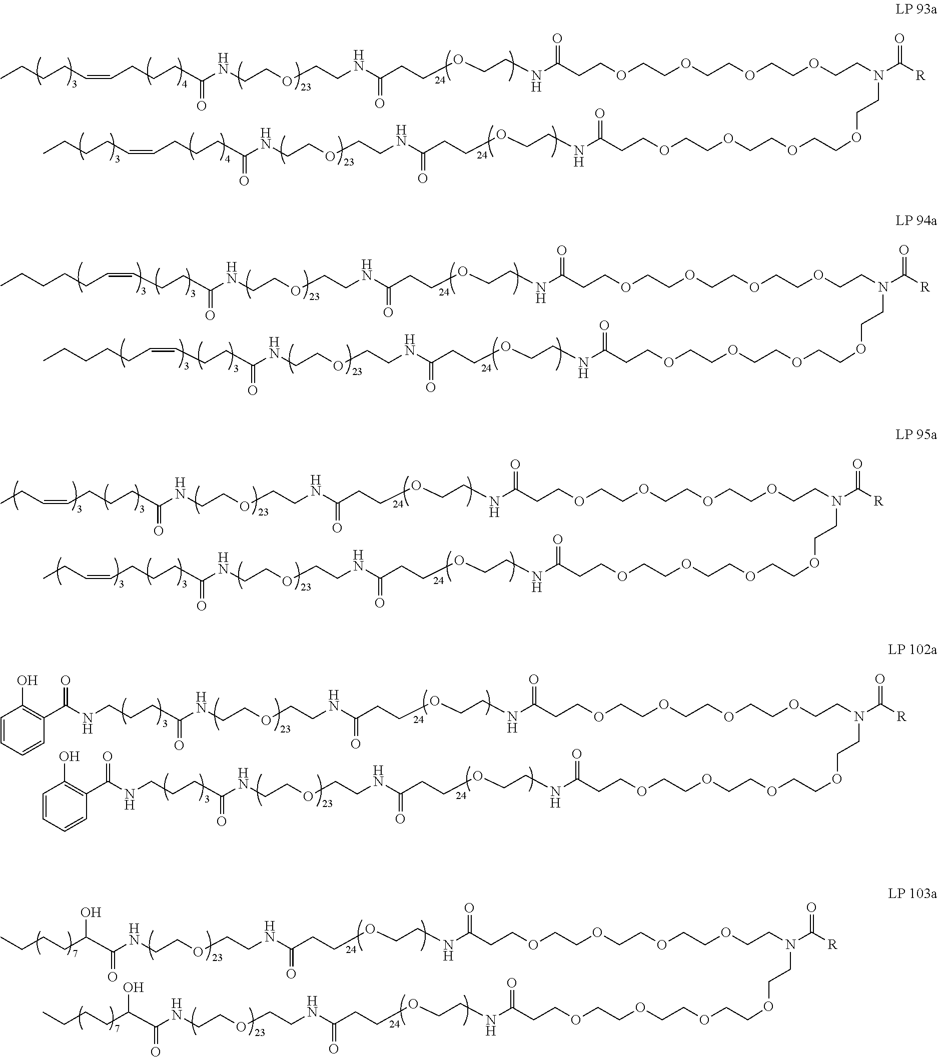 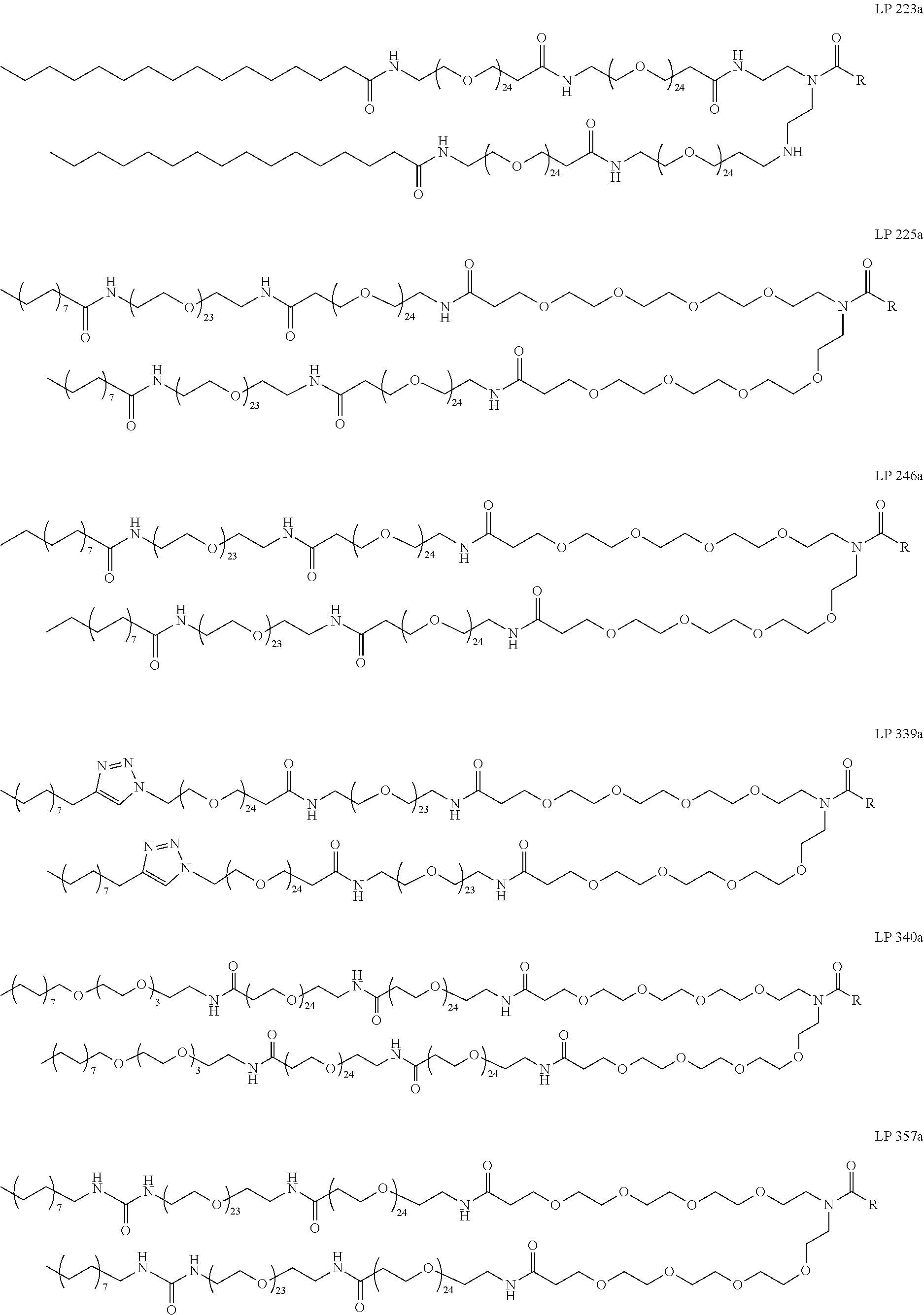 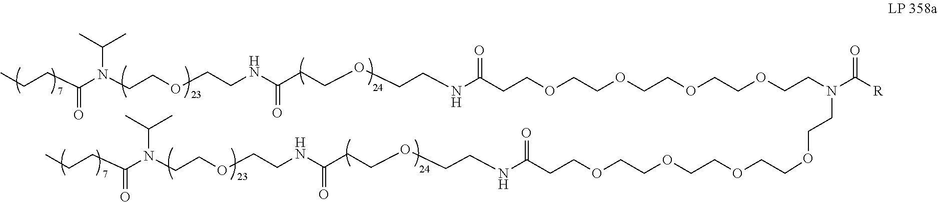
or a pharmaceutically acceptable salt of any of these compounds.

111. The compound or pharmaceutically acceptable salt of claim 92, wherein the compound of Formula (IV) is selected from the group consisting of: 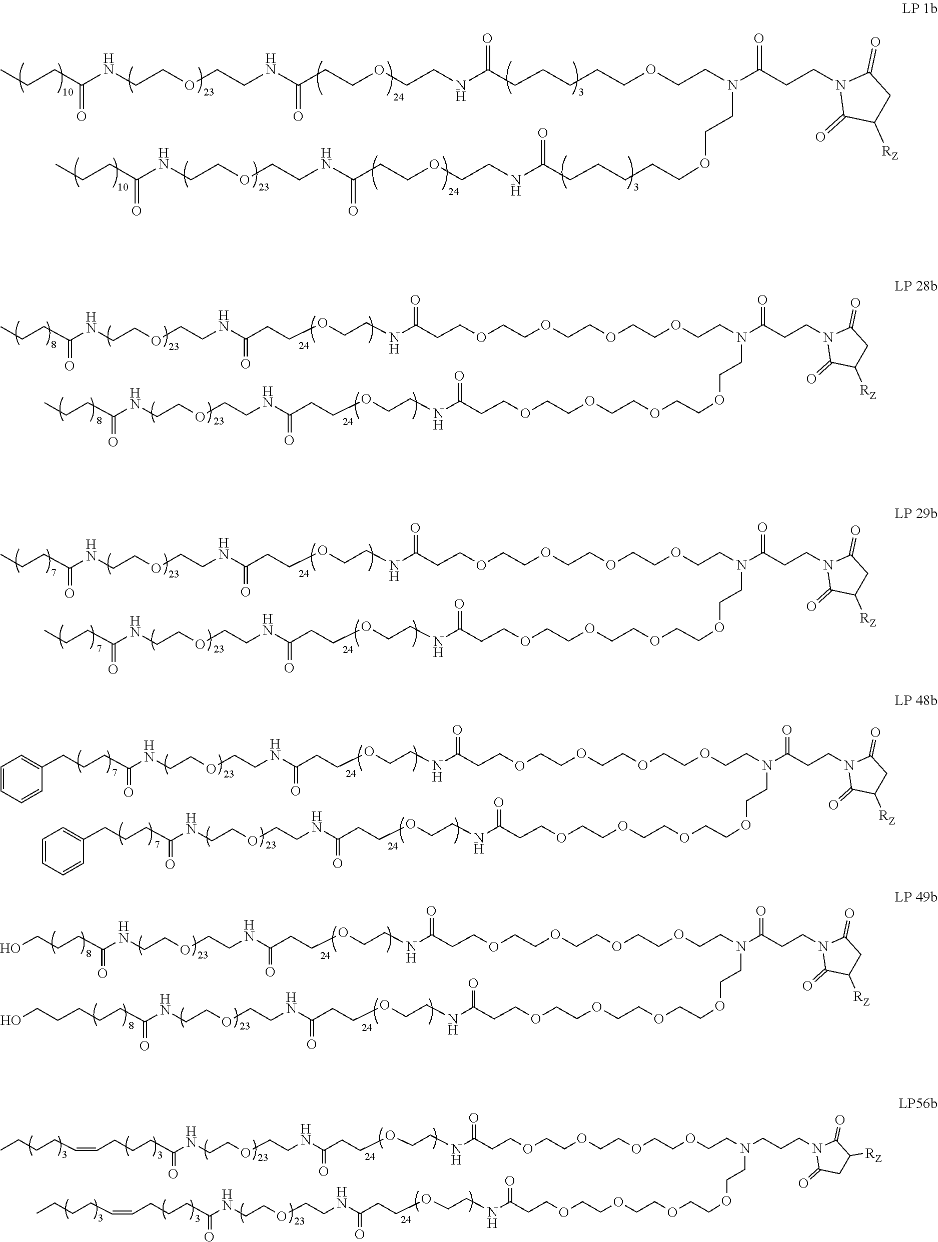 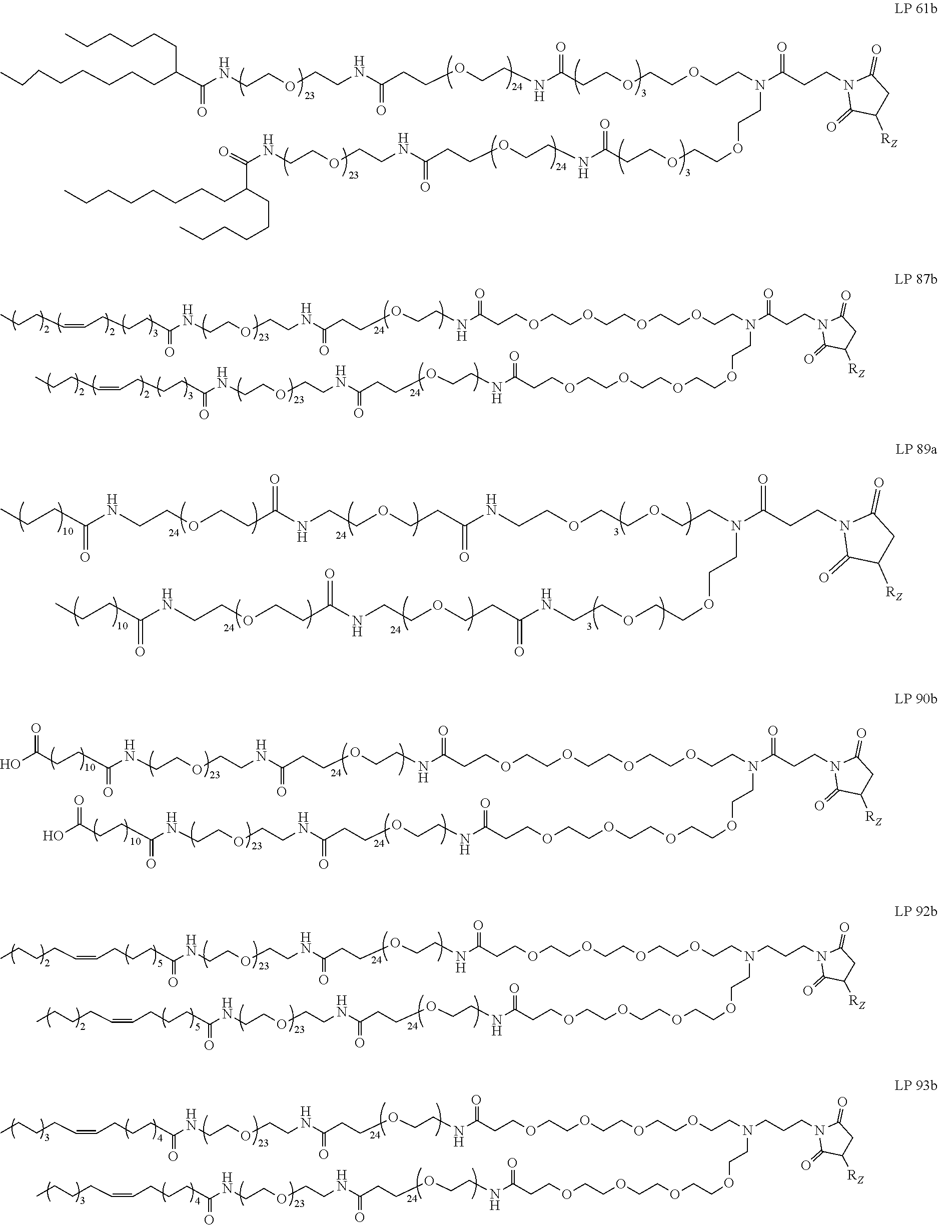 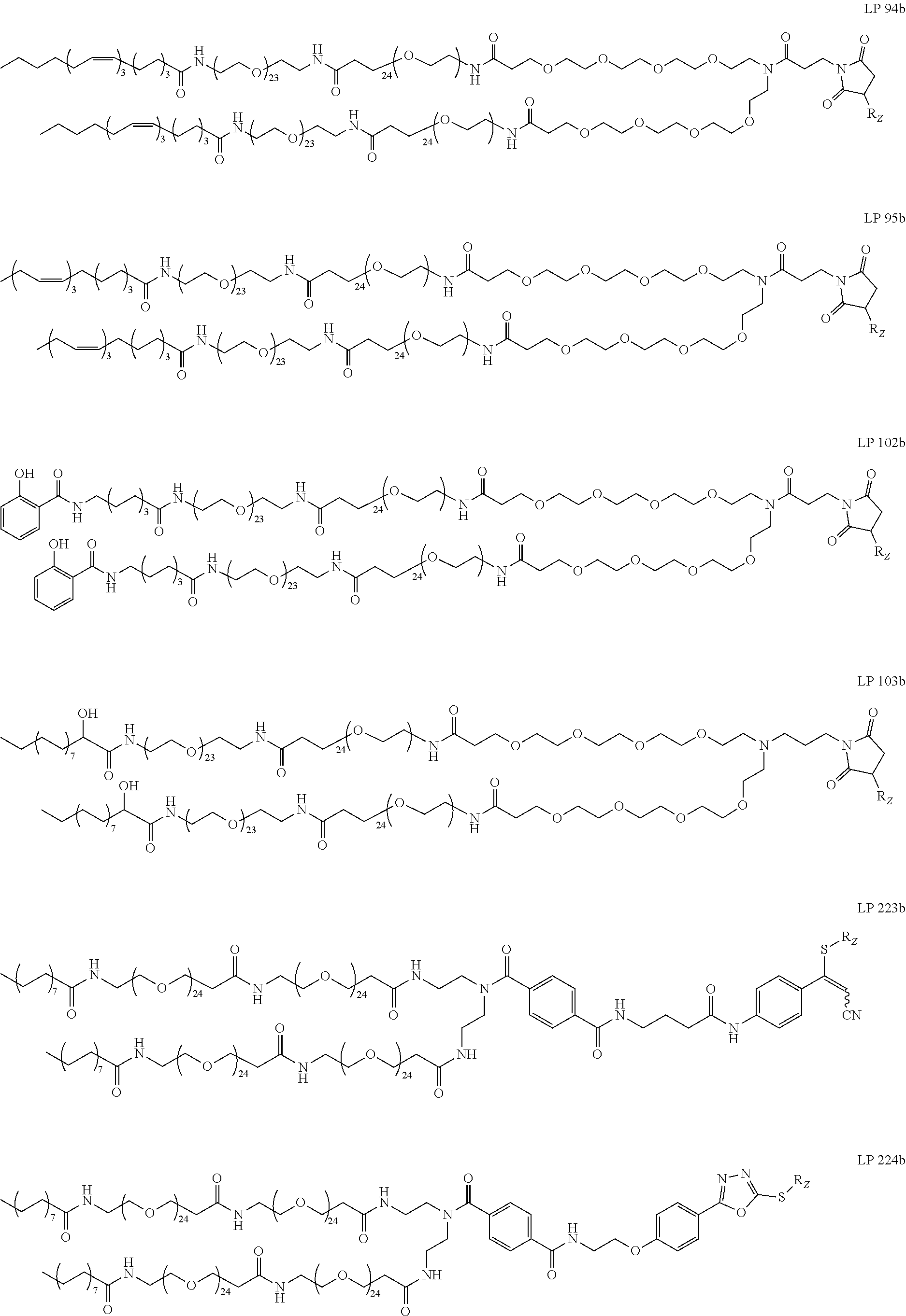 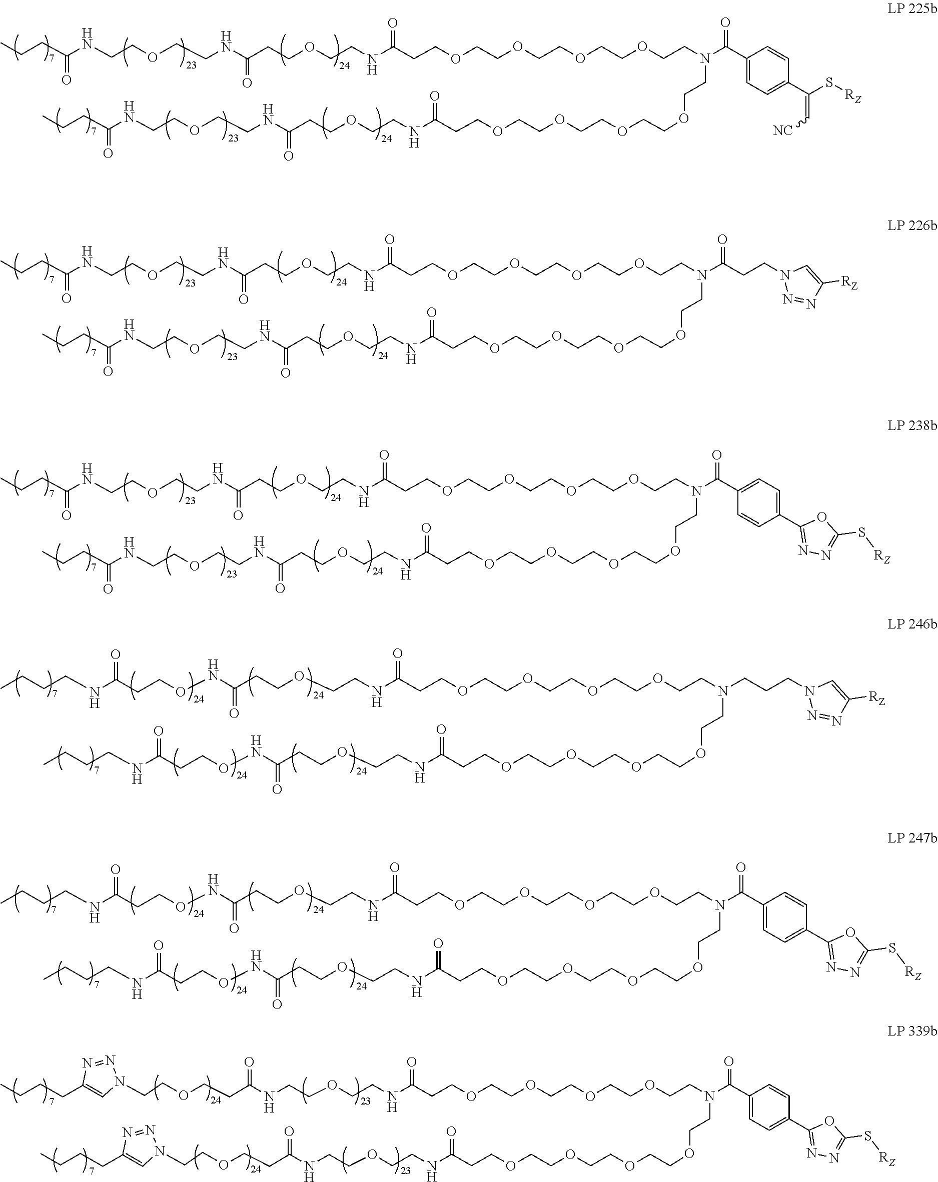 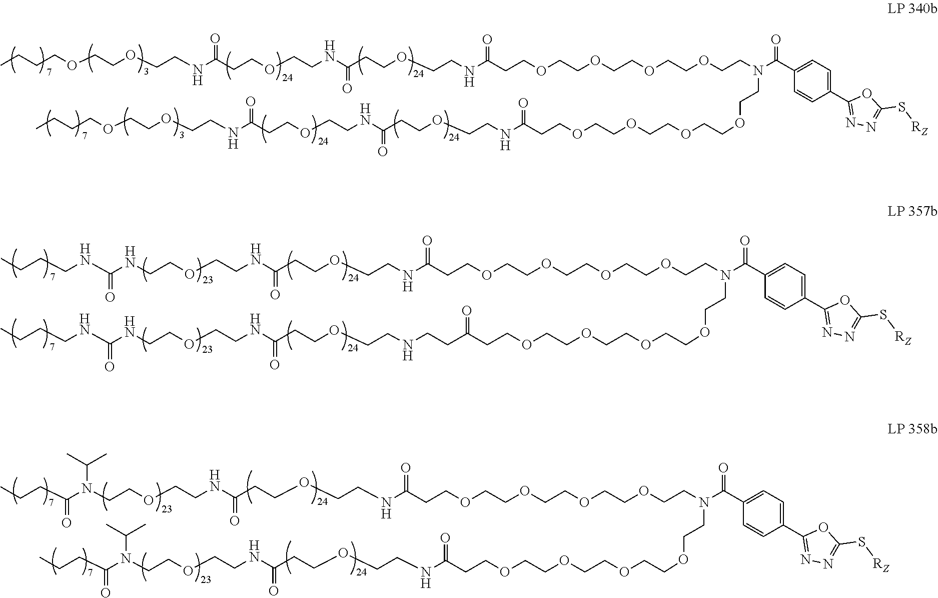
or a pharmaceutically acceptable salt of any of these compounds.

112. The compound or pharmaceutically acceptable salt of any one of claims 92-111, wherein the oligonucleotide-based agent is an RNAi agent.

113. A method of reducing a target gene expression in vivo, the method comprising introducing to a cell the compound or pharmaceutically acceptable salt of any one of claims 1-112, wherein the compound comprises an RNAi agent at least substantially complementary to the target gene.

114. The method of claim 113, wherein the cell is a skeletal muscle cell.

115. The method of claim 113, wherein the cell is an adipocyte.

116. The method of any one of claims 113-115, wherein the cell is within a subject.

117. The method of any one of claim 116, wherein the subject has been diagnosed with a disease or disorder that is treated, prevented or ameliorated by reducing expression of the target gene.

118. The method of claim 117, wherein the disease or disorder is a muscular dystrophy.

119. The method of claim 118, wherein the muscular dystrophy is selected from Duchenne muscular dystrophy, myotonic muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy.

120. Use of the compound of any one of claims 1-112 for the treatment, prevention, or amelioration of a disease or disorder.

121. The use of claim 120, wherein the disease or disorder is a muscular dystrophy.

122. The use of claim 121, wherein the muscular dystrophy is selected from Duchenne muscular dystrophy, myotonic muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy.

123. A compound of Formula (V): 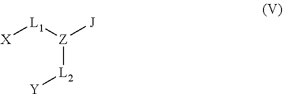
or a pharmaceutically acceptable salt thereof, wherein
J is L_{A5}-R_{X};
L_{A5} is a bond or a bivalent moiety connecting R_{X} to Z;
R_{X} is a reactive moiety for conjugation with an oligonucleotide-based agent;
Z is CH, phenyl, or N;
L₁ and L₂ are each independently linkers comprising at least about 5 PEG units; and
X and Y are each independently lipids comprising from about 10 to about 50 carbon atoms.

124. The compound or pharmaceutically acceptable salt of claim 123, wherein R_{X} is selected from the group consisting of 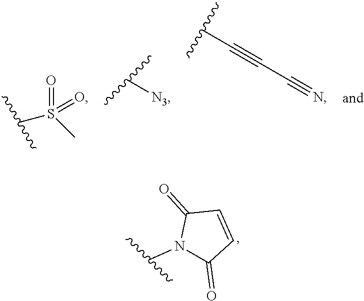
wherein [symbol] indicates a point of connection to L_{A5}.

125. The compound or pharmaceutically acceptable salt of claim 123 or 124, wherein L_{A5} is selected from the group consisting of:

| Name | Structure |
|---|---|
| Tether 1-5 | 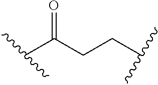 [SMILES: *CCC(*)=O] |
| Tether 2-5 | 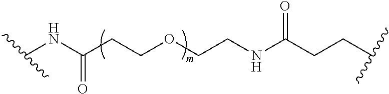 |
| Tether 3-5 | 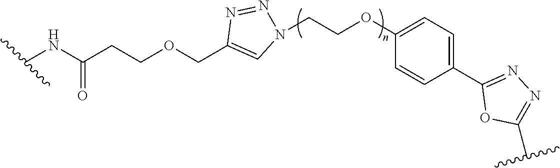 |
| Tether 4-5 | 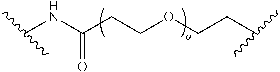 |
| Tether 5-5 | 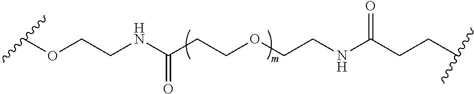 |
| Tether 6-5 | 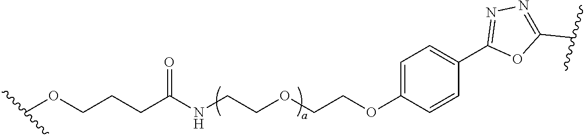 |
| Tether 7-5 | 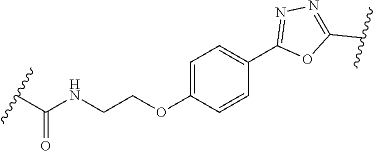 [SMILES: *C(=O)NCCOc1ccc(-c2nnc(*)o2)cc1] |
| Tether 8-5 | 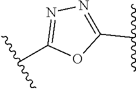 [SMILES: *c1nnc(*)o1] |
| Tether 9-5 | 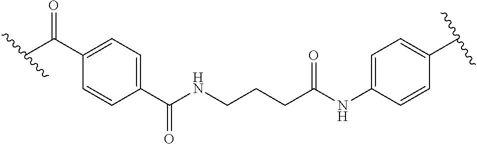 [SMILES: *C(=O)c1ccc(C(=O)NCCCC(=O)Nc2ccc(*)cc2)cc1] |
| Tether 10-5 | 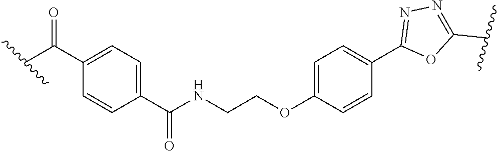 [SMILES: *C(=O)c1ccc(C(=O)NCCOc2ccc(-c3nnc(*)o3)cc2)cc1] |
| Tether 11-5 | 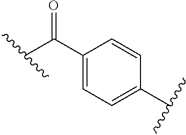 [SMILES: *C(=O)c1ccc(*)cc1] |
| Tether 12-5 | 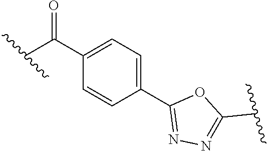 [SMILES: *C(=O)c1ccc(-c2nnc(*)o2)cc1] |
| Tether 13-5 | 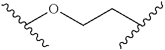 [SMILES: *CCO*] |

wherein each of m, n, o, and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to Z or R_{X}.

126. A compound, or a pharmaceutically acceptable salt thereof, selected from:

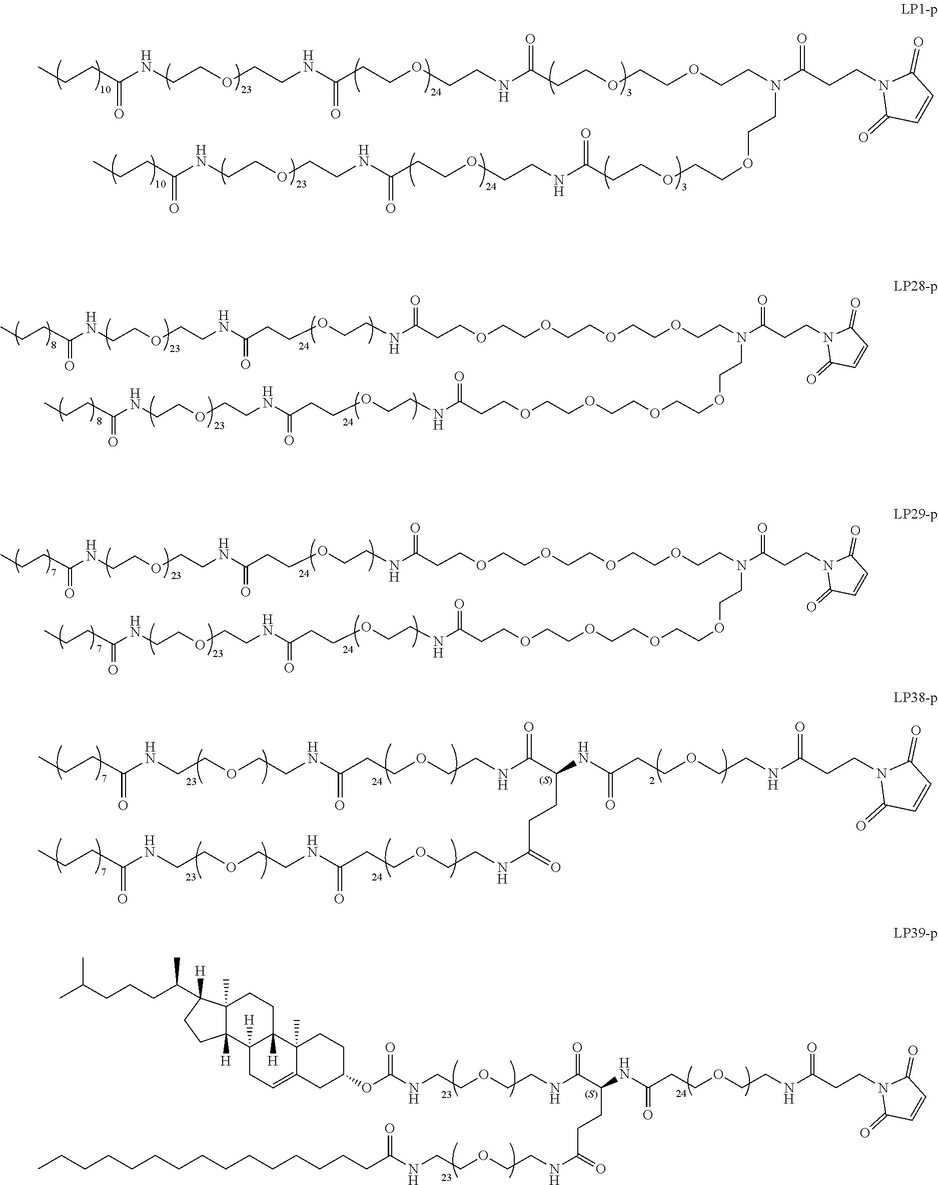

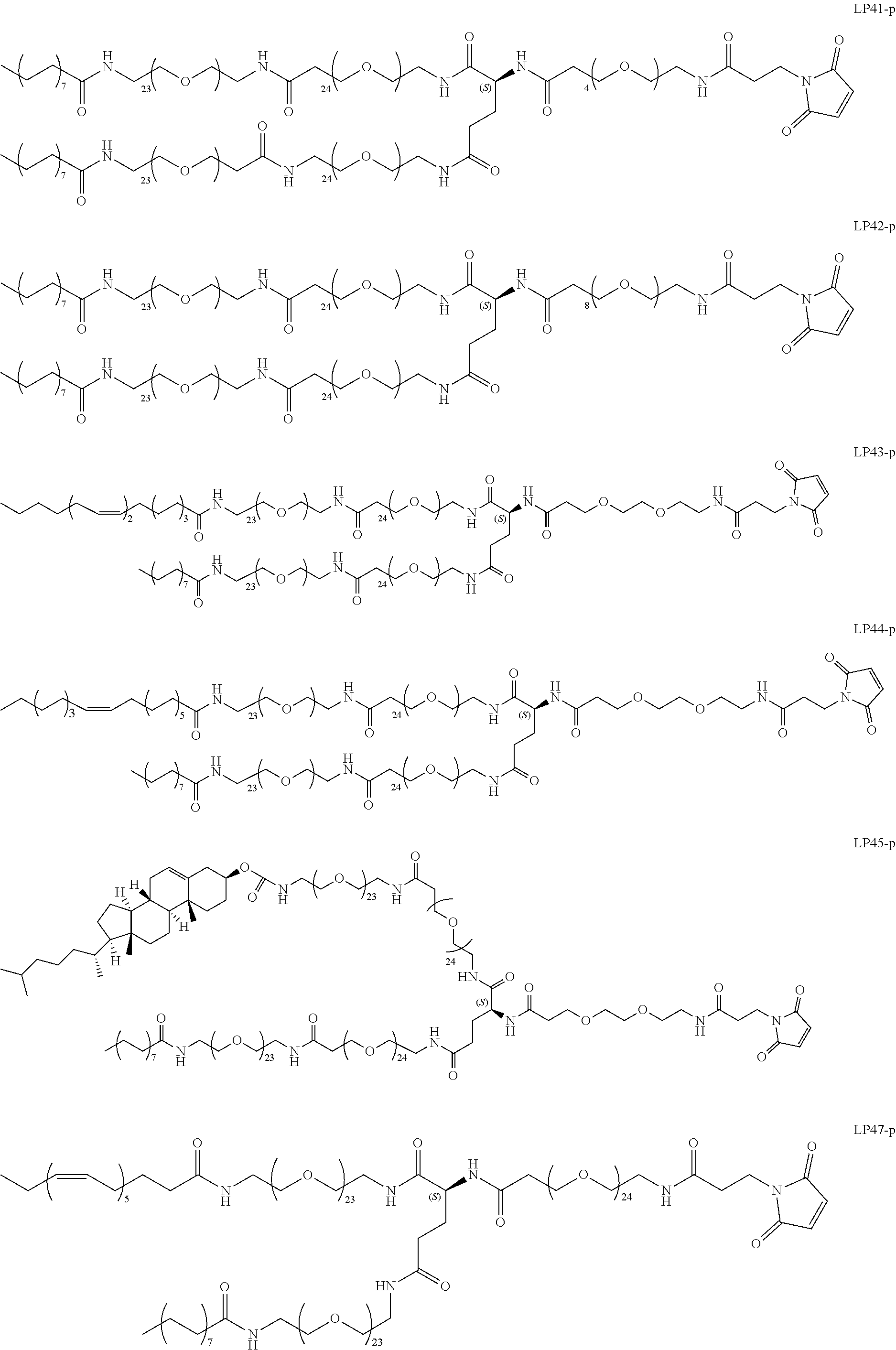

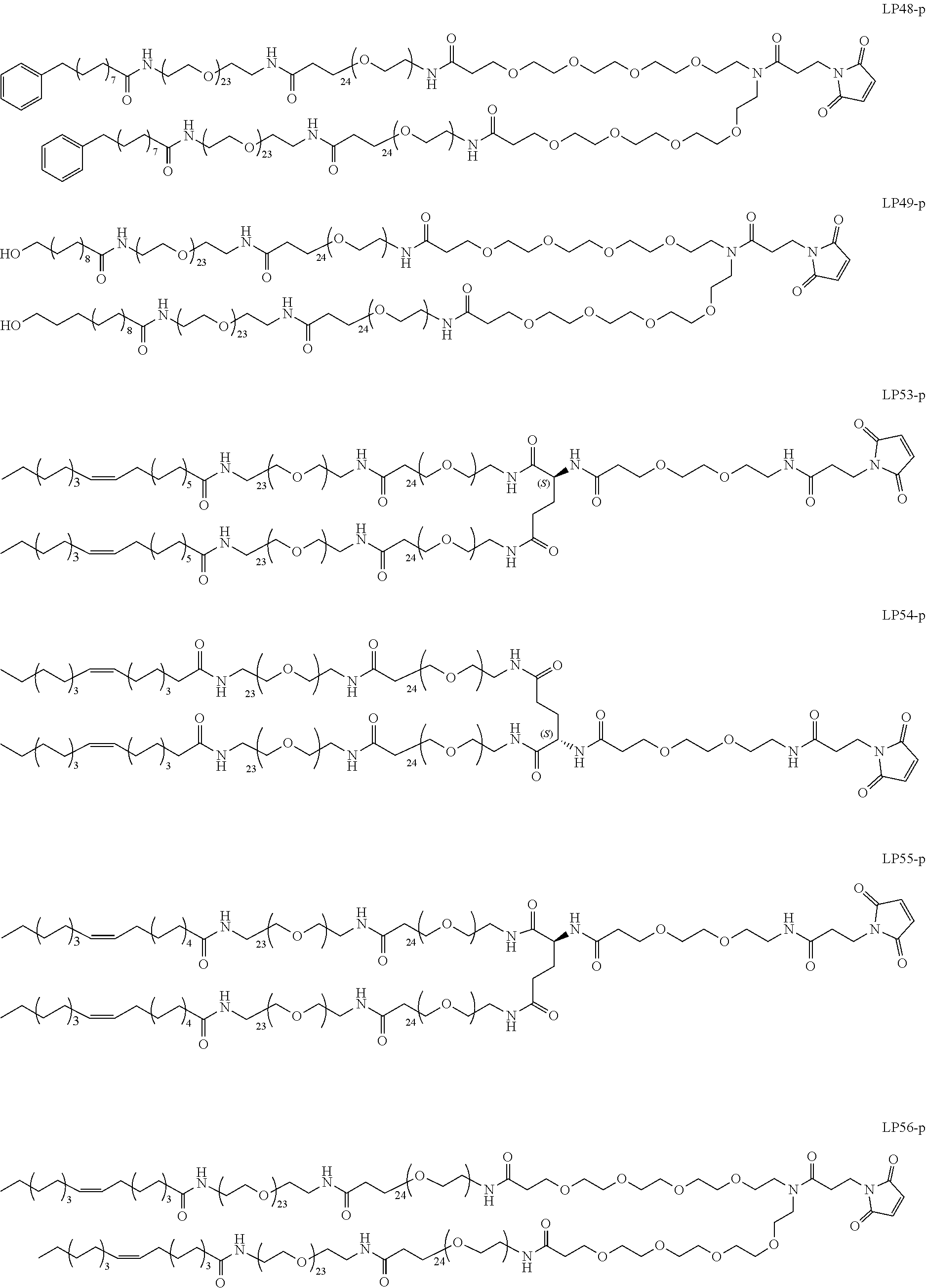

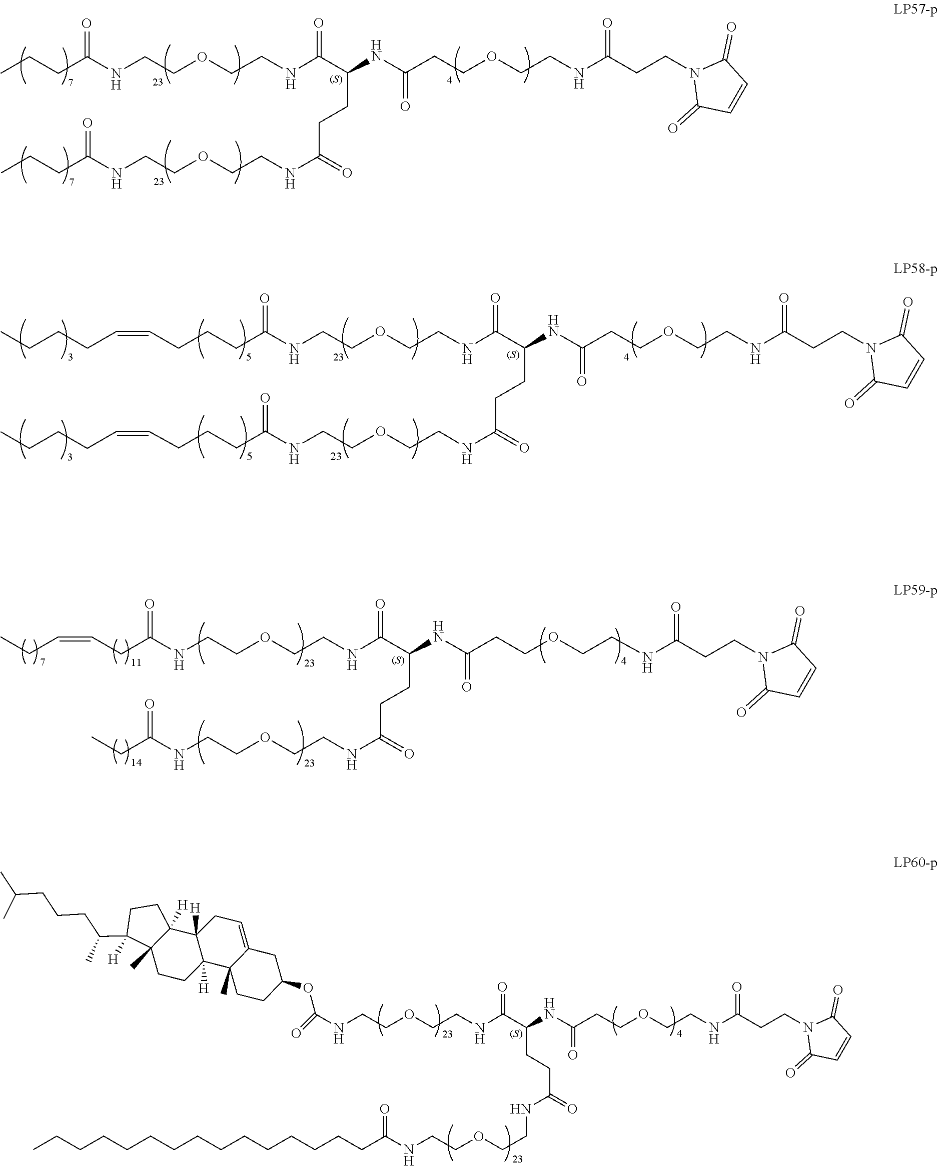

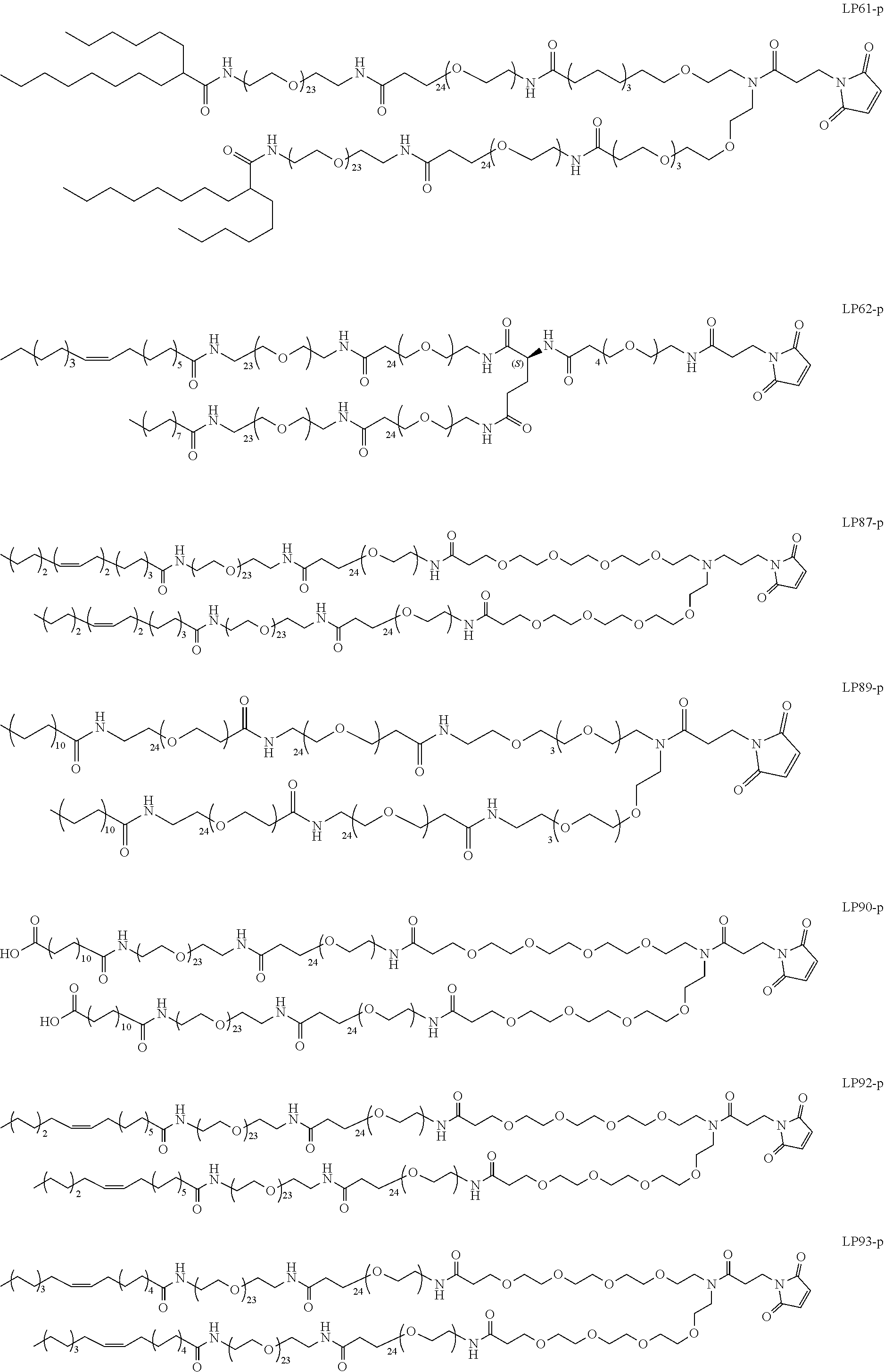

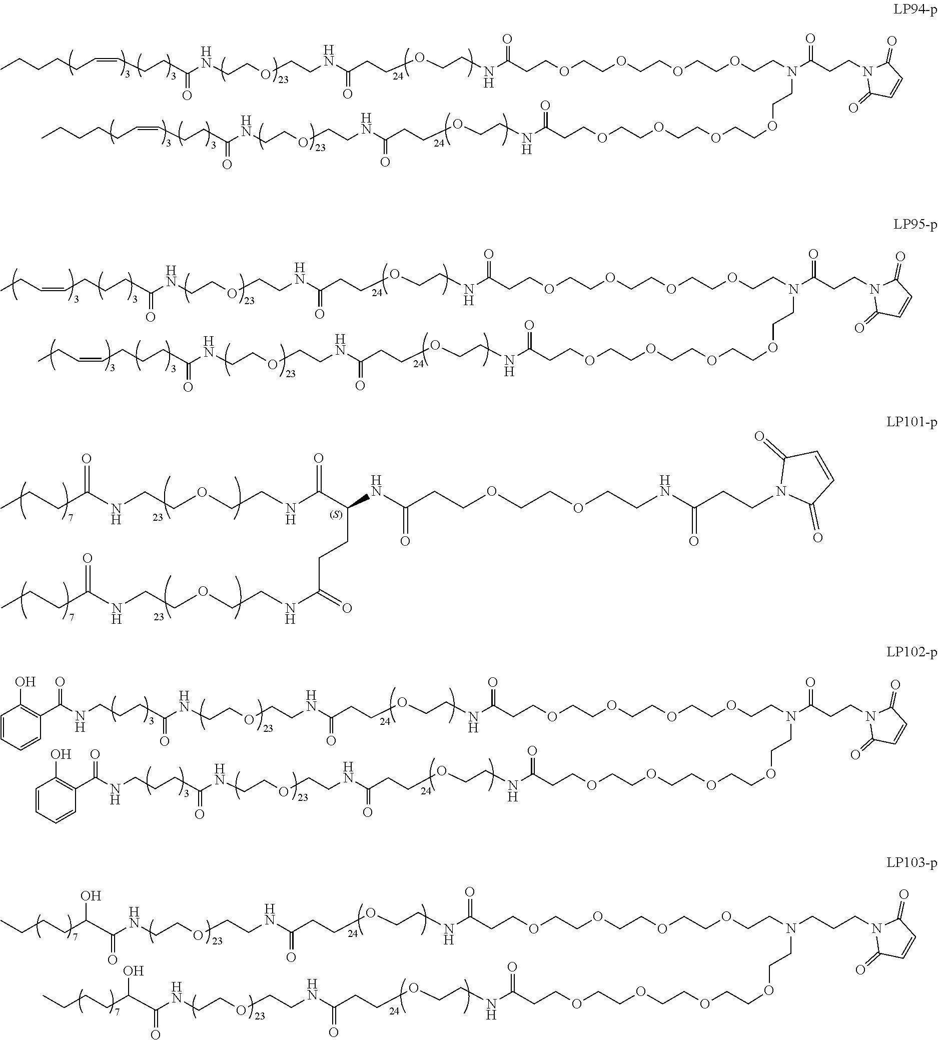

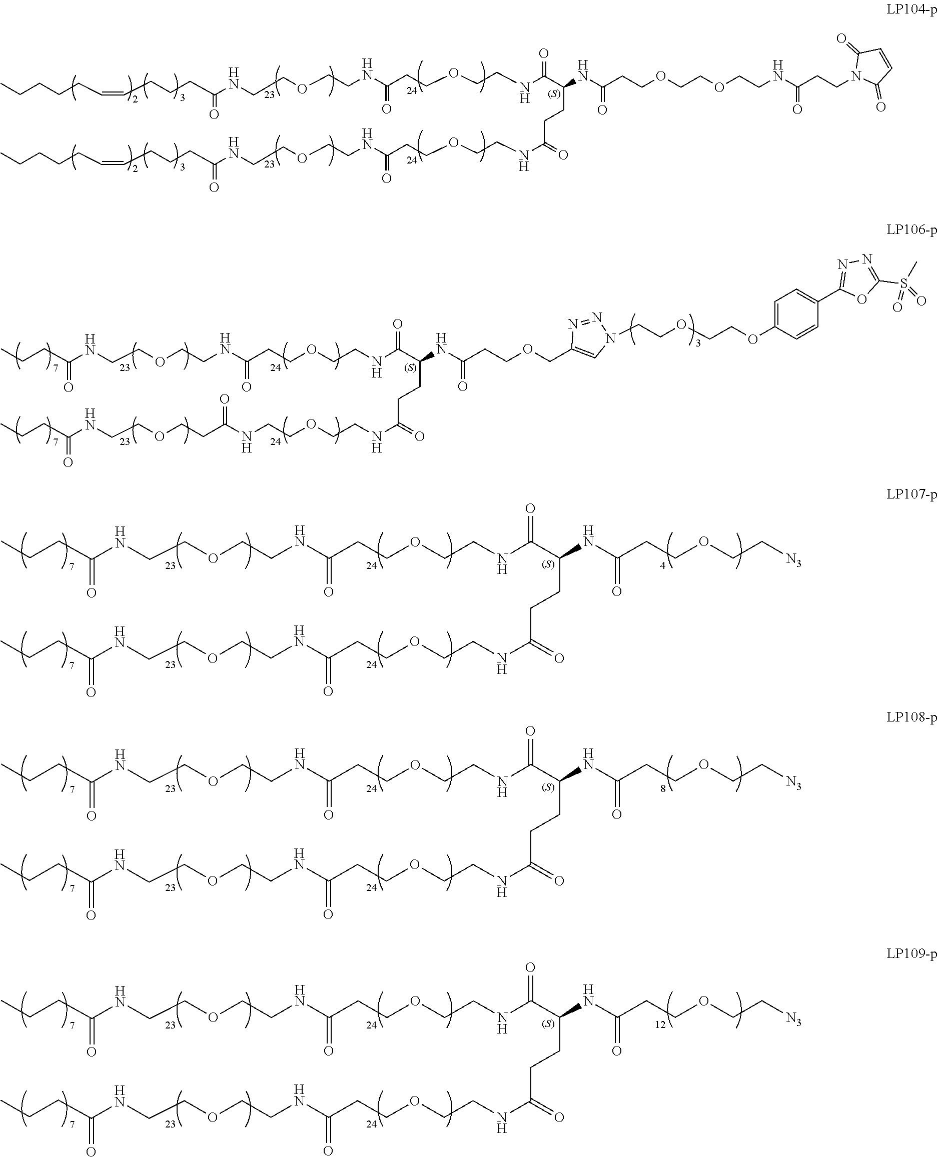

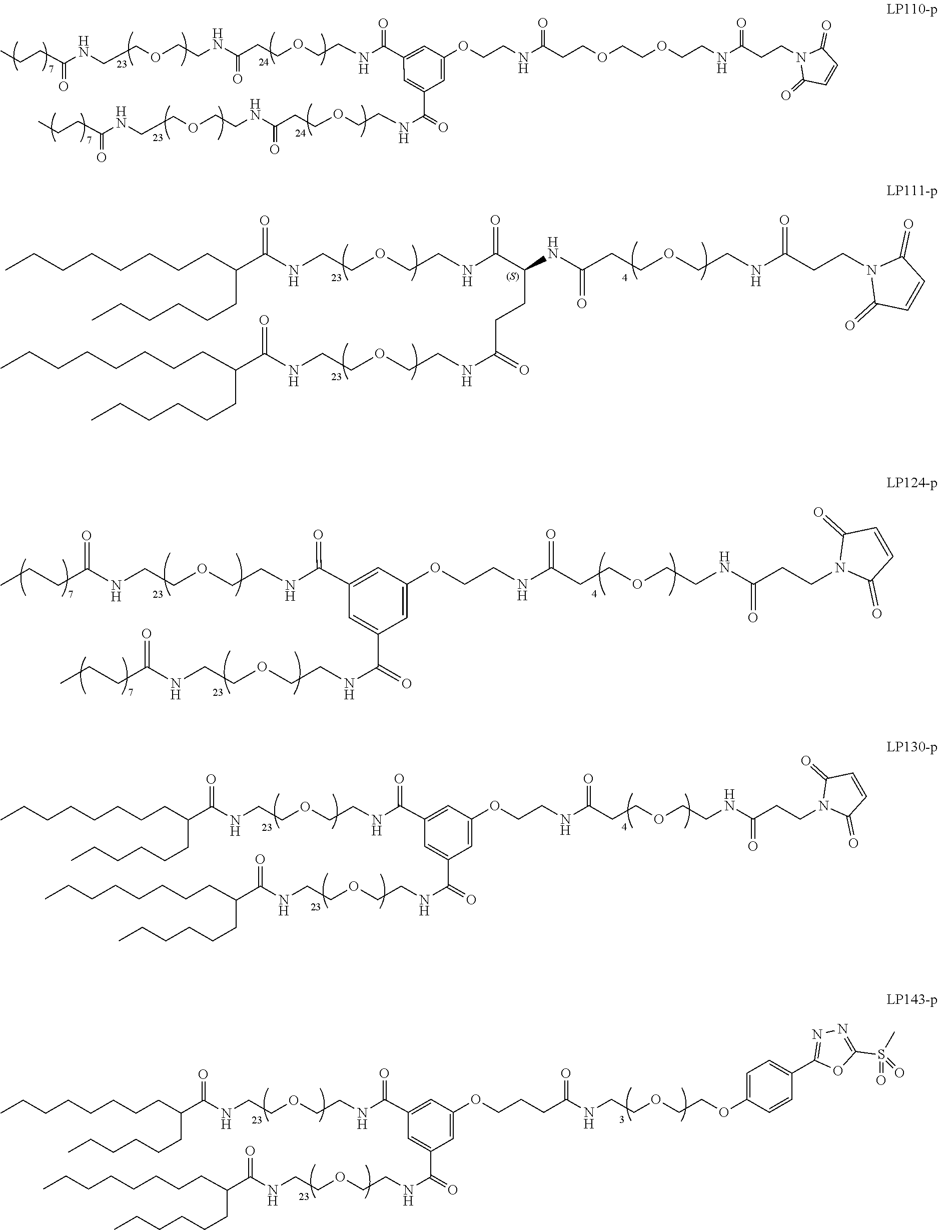

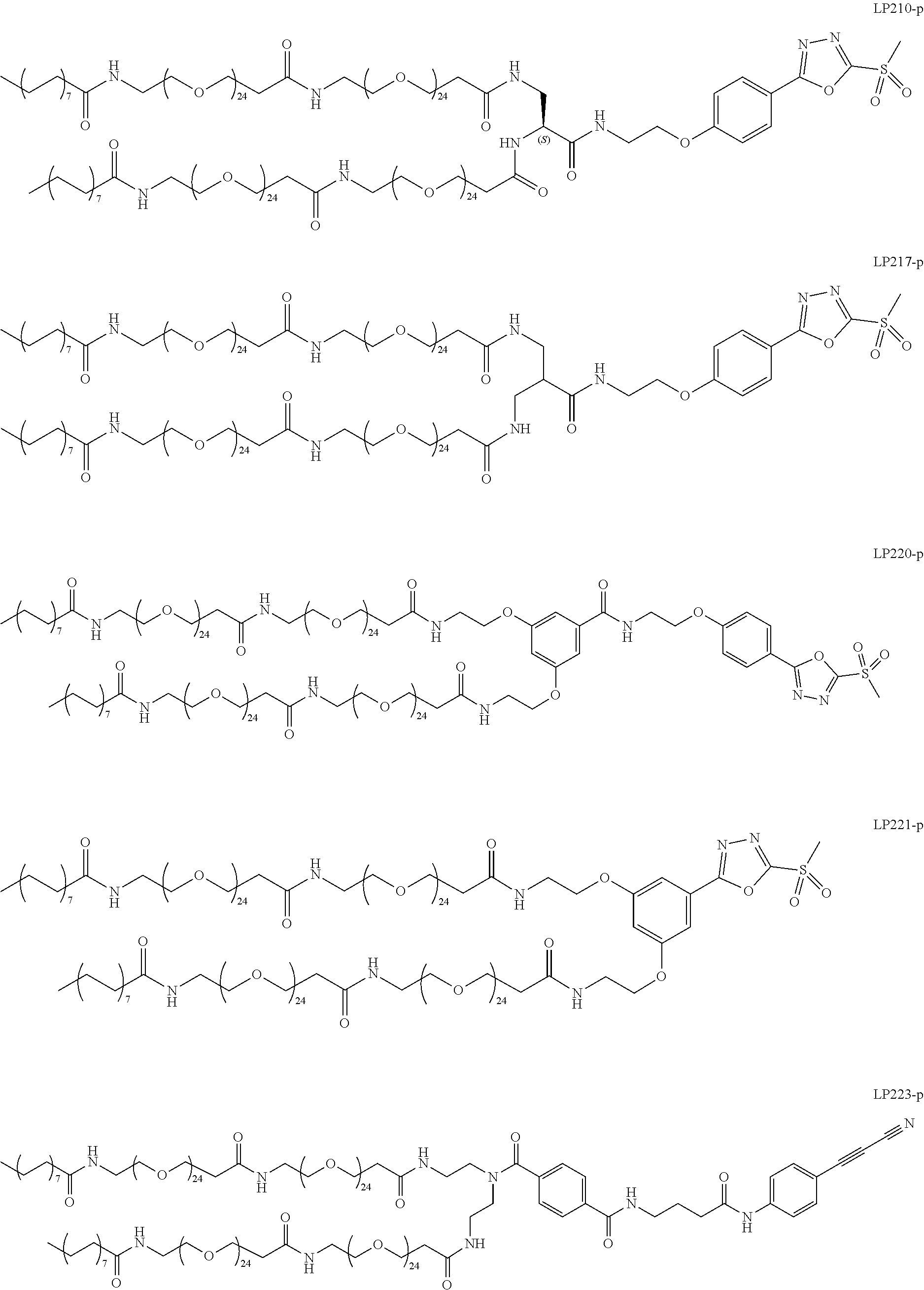

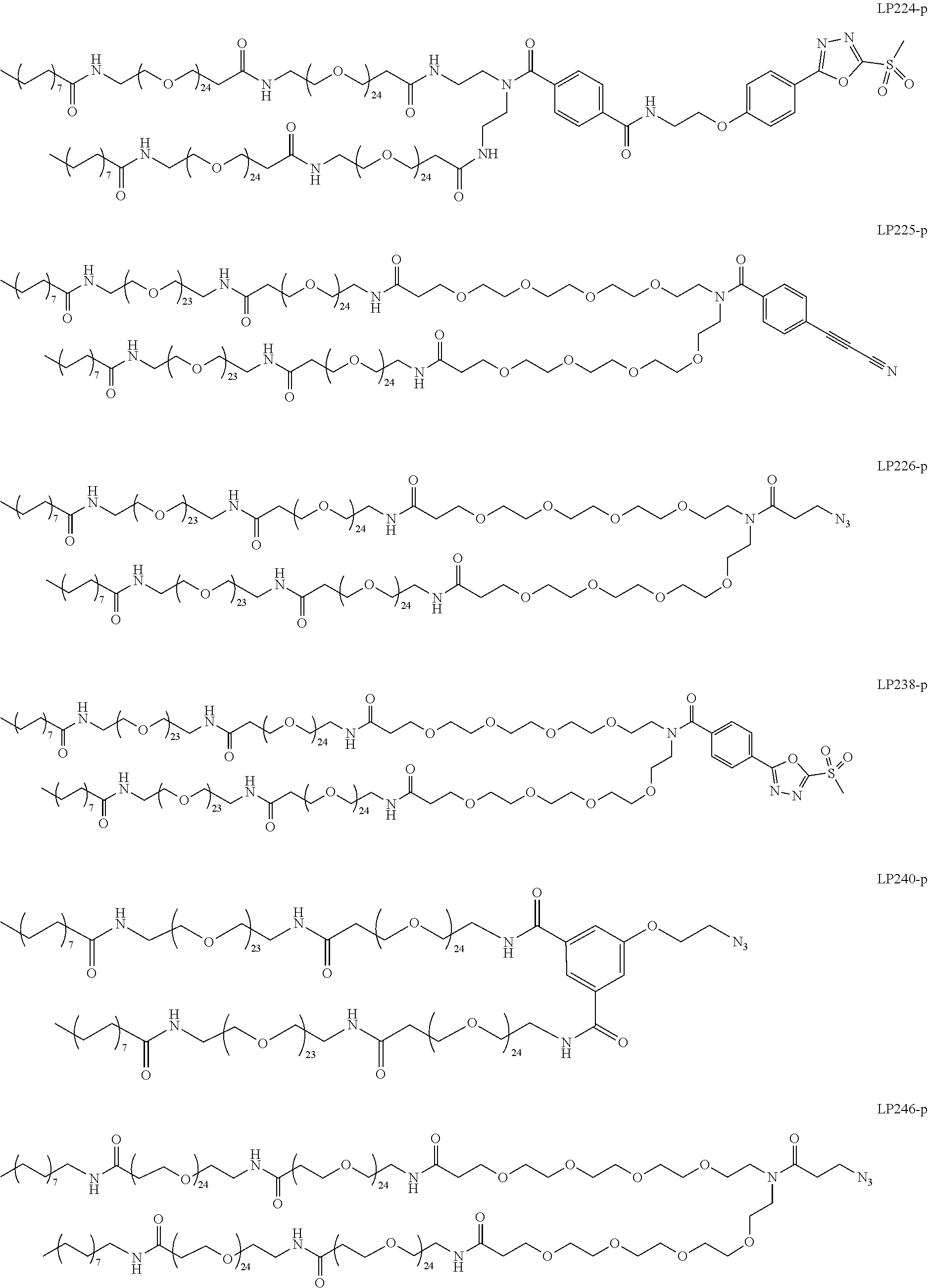

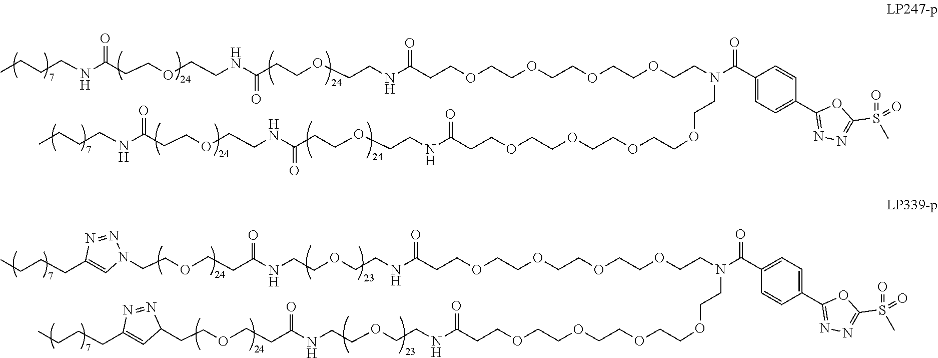

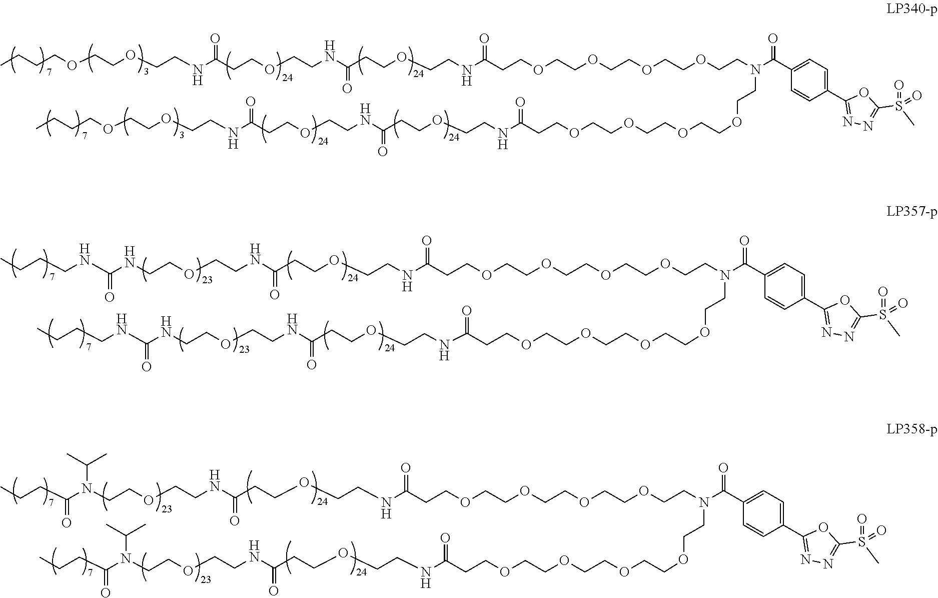

127. A method of making a compound of Formula (I): 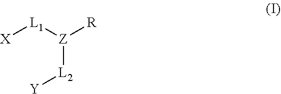
or a pharmaceutically acceptable salt thereof, wherein
R is L_{A}-R_{Z};
L_{A} is a bond or a bivalent moiety connecting R_{Z} to Z;
R_{Z} comprises an oligonucleotide-based agent;
Z is CH, phenyl or N;
L₁ and L₂ are each independently linkers comprising at least 5 PEG units; and
X and Y are each independently lipids comprising from about 10 to about 50 carbon atoms;
wherein the method comprises conjugating an RNAi agent comprising a first reactive moiety with a compound comprising a lipid and a second reactive moiety to form a compound of Formula (I).

128. The method of claim 127, wherein the first reactive moiety is selected from the group consisting of a disulfide and a propargyl group.

129. The method any one of claims 127 and 128, wherein the second reactive moiety is selected from the group consisting of maleimide, sulfone, azide, and alkyne.

130. The method of any one of claims 127-129, wherein the compound comprising a lipid is selected from any one of the compounds of claim 126.


SEQUENCE LISTING (10 sequences)

SEQ ID NO: 1 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatgacc

SEQ ID NO: 2 - RNA, 24 nt, synthetic construct
  modified_base 1: mod_base = OTHER; note = inverted abasic deoxyribonucleotide, 5' end (NH2-C6) modification with phosphorothioate linkage
  modified_base 1^2: mod_base = OTHER; note = phosphorothioate linkage
  modified_base 2: mod_base = gm
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-fluorouridine
  modified_base 13: mod_base = um
  modified_base 14: mod_base = gm
  modified_base 15: mod_base = cm
  modified_base 16: mod_base = um
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = cm
  modified_base 22: mod_base = OTHER; note = 2'-O-methyladenosine
  modified_base 22^23: mod_base = OTHER; note = phosphorothioate linkage
  modified_base 23: mod_base = OTHER; note = inverted abasic deoxyribonucleotide
  modified_base 23^24: mod_base = OTHER; note = C6-SS-C6 linkage
  modified_base 24: mod_base = OTHER; note = 2 -deoxythymidine
  source 1..24: mol_type = other RNA
nggtcatgatcttgctgtaacant

SEQ ID NO: 3 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatgacc

SEQ ID NO: 4 - RNA, 25 nt, synthetic construct
  modified_base 1: mod_base = OTHER; note = inverted abasic deoxyribonucleotide, 5' end (NH2-C6) modification with phosphorothioate linkage
  modified_base 1^2: mod_base = OTHER; note = phosphorothioate linkage
  modified_base 2: mod_base = gm
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-fluorouridine
  modified_base 13: mod_base = um
  modified_base 14: mod_base = gm
  modified_base 15: mod_base = cm
  modified_base 16: mod_base = um
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = cm
  modified_base 22: mod_base = OTHER; note = 2'-O-methyladenosine
  modified_base 22^23: mod_base = OTHER; note = phosphorothioate linkage
  modified_base 23: mod_base = OTHER; note = inverted abasic deoxyribonucleotide
  modified_base 24: mod_base = OTHER; note = 2 -O-propargyluridine
  modified_base 25: mod_base = OTHER; note = 2 -deoxythymidine
  source 1..25: mol_type = other RNA
nggtcatgatcttgctgtaacantt

SEQ ID NO: 5 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatgacc

SEQ ID NO: 6 - RNA, 24 nt, synthetic construct
  modified_base 1: mod_base = OTHER; note = inverted abasic deoxyribonucleotide, 5' end (NH2-C6) modification with phosphorothioate linkage
  modified_base 1^2: mod_base = OTHER; note = phosphorothioate linkage
  modified_base 2: mod_base = gm
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-fluorouridine
  modified_base 13: mod_base = um
  modified_base 14: mod_base = gm
  modified_base 15: mod_base = cm
  modified_base 16: mod_base = um
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = cm
  modified_base 22: mod_base = OTHER; note = 2'-O-methyladenosine
  modified_base 22^23: mod_base = OTHER; note = phosphorothioate linkage
  modified_base 23: mod_base = OTHER; note = inverted abasic deoxyribonucleotide
  modified_base 24: mod_base = OTHER; note = 2 -O-propargyluridine
  source 1..24: mol_type = other RNA
nggtcatgatcttgctgtaacant

SEQ ID NO: 7 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatgacc

SEQ ID NO: 8 - RNA, 23 nt, synthetic construct
  modified_base 1: mod_base = OTHER; note = inverted abasic deoxyribonucleotide, 5' end (NH2-C6) modification with phosphorothioate linkage
  modified_base 1^2: mod_base = OTHER; note = phosphorothioate linkage
  modified_base 2: mod_base = gm
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-fluorouridine
  modified_base 13: mod_base = um
  modified_base 14: mod_base = gm
  modified_base 15: mod_base = cm
  modified_base 16: mod_base = um
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = cm
  modified_base 22: mod_base = OTHER; note = 2 -O-propargyladenosine
  modified_base 22^23: mod_base = OTHER; note = phosphorothioate linkage
  modified_base 23: mod_base = OTHER; note = inverted abasic deoxyribonucleotide
  source 1..23: mol_type = other RNA
nggtcatgatcttgctgtaacan

SEQ ID NO: 9 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 3: mod_base = um
  modified_base 4: mod_base = OTHER; note = 2'-fluorouridine
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 15^16: mod_base = OTHER; note = phosphorothioate linkage
  modified_base 16: mod_base = OTHER; note = 2'-fluoroadenosine
  modified_base 16^17: mod_base = OTHER; note = phosphorothioate linkage
  modified_base 17: mod_base = um
  modified_base 17^18: mod_base = OTHER; note = phosphorothioate linkage
  modified_base 18: mod_base = OTHER; note = 2'-fluoroguanosine
  modified_base 18^19: mod_base = OTHER; note = phosphorothioate linkage
  modified_base 19: mod_base = OTHER; note = 2'-O-methyladenosine
  modified_base 19^20: mod_base = OTHER; note = phosphorothioate linkage
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatgacc

SEQ ID NO: 10 - RNA, 24 nt, synthetic construct
  modified_base 1: mod_base = OTHER; note = inverted abasic deoxyribonucleotide, 5' end (NH2-C6) modification with phosphorothioate linkage
  modified_base 1^2: mod_base = OTHER; note = phosphorothioate linkage
  modified_base 2: mod_base = gm
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-fluorouridine
  modified_base 13: mod_base = um
  modified_base 14: mod_base = gm
  modified_base 15: mod_base = cm
  modified_base 16: mod_base = um
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = cm
  modified_base 22: mod_base = OTHER; note = 2'-O-methyladenosine
  modified_base 22^23: mod_base = OTHER; note = phosphorothioate linkage
  modified_base 23: mod_base = OTHER; note = inverted abasic deoxyribonucleotide
  modified_base 24: mod_base = OTHER; note = LA2
  source 1..24: mol_type = other RNA
nggtcatgatcttgctgtaacann